(19) United States
(12) Patent Application Publication
(10) Pub. No.: US 2024/0376466 A1
(43) Pub. Date: Nov. 14, 2024

(54) GENOME EDITING COMPOSITIONS AND METHODS FOR TREATMENT OF FANCONI ANEMIA

(71) Applicant: Prime Medicine, Inc., Cambridge, MA (US)

(72) Inventors: Jennifer L. GORI, Jamaica Plain, MA (US); David WATERMAN, Framingham, MA (US); Dewi HARJANTO, Brookline, MA (US)

(21) Appl. No.: 18/653,321

(22) Filed: May 2, 2024

Related U.S. Application Data
(63) Continuation of application No. PCT/US2022/079257, filed on Nov. 3, 2022.
(60) Provisional application No. 63/275,702, filed on Nov. 4, 2021.

(51) Int. Cl. C12N 15/11; C12N 9/22

(52) U.S. Cl. CPC C12N 15/11; C12N 9/22; C12N 2310/20

(57) ABSTRACT

Provided herein are compositions and methods of using prime editing systems comprising prime editors and prime editing guide RNAs for treatment of genetic disorders.

### CROSS-REFERENCE

This application is a continuation of International Application No. PCT/US2022/079257, filed Nov. 3, 2022, which claims the benefit of U.S. Provisional Application No. 63/275,702, filed Nov. 4, 2021, each of which applications are incorporated herein by reference in their entirety.

### SEQUENCE LISTING

The instant application contains a Sequence Listing which has been submitted electronically in XML format and is hereby incorporated by reference in its entirety. Said XML copy, created on Dec. 19, 2022, is named 59761-746_601_SL.xml and is 3,575,873 bytes in size.

### BACKGROUND

Fanconi Anemia (FA) is an inherited autosomal recessive DNA repair disorder characterized by congenital abnormalities, cancer predisposition, and progressive bone marrow failure (BMF). Fanconi anemia occurs in about 1 in 160,000 live births worldwide. FA can be caused by mutations in one or more of 23 genes whose protein products are members of or associated with the Fanconi anemia DNA repair complex called FA core complex. The FA core complex responds to and repairs DNA breaks that occur naturally during cellular replication or in response to radiation or DNA crosslinking agents via activation of a FA/BRCA DNA repair pathway. The pathway can promote homologous recombination (HR) repair, regulate cytokinesis and pathway disruption can result in increased binucleate bone marrow cells and apoptosis.

Based on the mutation in one or more of the 23 genes, at least 19 genetic subtypes of FA have been distinguished: FA-A, -B, -C, -D1, -D2, -E, -F, -G, -I, -J, -L, -M, -N, -O, and -P, -Q, -R, -S, and -T. The majority of patients (˜85%) belong to the subtype FA-A (˜60%) that results from inherited pathogenic variants of FANCA gene, subtype FA-C(15%) that results from inherited pathogenic variants of FANCC gene, or subtype FA-G (A10%) that results from inherited pathogenic variants of FANG, while a minority (˜15%) is distributed over the remaining 16 subtypes, with relative prevalence between <1 and 5%. FA-C patients are likely to undergo BMF in the first decade of life and allogeneic hematopoietic stem cell transplantation (allo-HSCT) is currently the only curative option. However, the sensitivity of FA-C patient cells to DNA damage complicates allo-HSCT because of the reliance on alkylating agents and radiation for pre-transplant conditioning. In some cases, a first line therapy of androgen and growth factors are used but only 50% to 75% of patients respond to this treatment. FA-C patients demonstrate higher incidence and more rapid emergence of BMF compared to other groups at a median age of 2.7 years.

### SUMMARY

The present disclosure provides compositions and methods for correcting mutations associated with Fanconi anemia of complementation group C (FA-C), and compositions and methods for treatment of Fanconi anemia of complementation group C (FA-C).

Provided herein, in some embodiments, are methods and compositions for prime editing of alterations in a target sequence in a target gene, for example, an FANCC gene. The target FANCC gene can comprise double stranded DNA. As exemplified in FIG. 1, in an embodiment, the target gene is edited by prime editing.

Without wishing to be bound by any particular theory, the prime editing process can search specific targets and edit endogenous sequences in a target gene, e.g., the FANCC gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex can then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target FANCC gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target FANCC gene.

In one aspect, provided herein is a prime editing guide RNA (PEgRNA) or a nucleic acid encoding the PEgRNA, wherein the PEgRNA comprises: a) a spacer that is complementary to a search target sequence on a first strand of a FANCC gene wherein the spacer comprises at its 3′ end SEQ ID NO: 136; b) a gRNA core capable of binding to a Cas9 protein; and c) an extension arm comprising: i) an editing template that comprises a region of complementarity to an editing target sequence on a second strand of the FANCC gene, and ii) a primer binding site (PBS) that comprises at its 5′ end a sequence that is a reverse complement of nucleotides 10-14 of SEQ ID NO: 136, wherein the first strand and second strand are complementary to each other, wherein the editing target sequence on the second strand comprises or is complementary to a portion of the FANCC gene comprising a c.67del mutation, and wherein the editing template encodes an insertion of a guanine nucleotide at a site corresponding to the c.67del mutation compared to the editing target sequence. In some embodiments, the spacer comprises at its 3′ end any one of SEQ ID NOs: 137-141. In some embodiments, the spacer comprises at its 3′ end SEQ ID NO: 139. In some embodiments, the editing template comprises SEQ ID NO: 157 at its 3′ end. In some embodiments, the editing template comprises at its 3′ end SEQ ID NO: 159, 160, 162, 163, 165, 166, 168-181, 183, 184, 186, 187, or 189-194. In some embodiments, the editing template comprises SEQ ID NO: 158 at its 3′ end and encodes an AGG-to-AAG PAM silencing edit. In some embodiments, the editing template comprises at its 3′ end SEQ ID NO: 161, 164, 167, 182, 185, or 188. In some embodiments, the editing template is 10 to 40 nucleotides in length. In some embodiments, the editing template is 10 to 32 nucleotides in length. In some embodiments, the editing template is 12 to 16 nucleotides in length. In some embodiments, the PBS comprises at its 5′ end a sequence corresponding to sequence number 142. In some embodiments, the PBS comprises any one of sequence numbers 142-146, or any one of SEQ ID NOs: 147-156. In some embodiments, the PBS is 8 to 14 nucleotides in length. In some embodiments, the PBS is 14 nucleotides in length. In some embodiments, the gRNA core comprises SEQ ID NO: 3666. In some embodiments, a PEgRNA sequence is selected from any one of SEQ ID NOs: 219-322 or 3592-3603. In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In one aspect, provided herein is a prime editing guide RNA (PEgRNA), or a nucleic acid encoding the PEgRNA, wherein the PEgRNA comprises: a) a spacer comprising at its 3′ end SEQ ID NO: 136; b) a gRNA core capable of binding to a Cas9 protein; and c) an extension arm comprising: i) an editing template comprising at its 3′ end SEQ ID NO: 157 or 158, and ii) a primer binding site (PBS) comprising at its 5′ end a sequence that is a reverse complement of nucleotides 10-14 of SEQ ID NO: 136. In some embodiments, the spacer comprises at its 3′ end any one of SEQ ID NOs: 137-141. In some embodiments, the spacer comprises at its 3′ end SEQ ID NO: 139. In some embodiments, the editing template comprises SEQ ID NO: 157 at its 3′ end. In some embodiments, the editing template comprises at its 3′ end SEQ ID NO: 159, 160, 162, 163, 165, 166, 168-181, 183, 184, 186, 187, or 189-194. In some embodiments, the editing template comprises SEQ ID NO: 158 at its 3′ end and encodes an AGG-to-AAG PAM silencing edit. In some embodiments, the editing template comprises at its 3′ end SEQ ID NO: 161, 164, 167, 182, 185, or 188. In some embodiments, the editing template is 10 to 40 nucleotides in length. In some embodiments, the editing template is 10 to 32 nucleotides in length. In some embodiments, the editing template is 12 to 16 nucleotides in length. In some embodiments, the PBS comprises at its 5′ end a sequence corresponding to sequence number 142. In some embodiments, the PBS comprises any one of sequence numbers 142-146, or any one of SEQ ID NOs: 147-156. In some embodiments, the PBS is 8 to 14 nucleotides in length. In some embodiments, the PBS is 14 nucleotides in length. In some embodiments, the gRNA core comprises SEQ ID NO: 3666. In some embodiments, a PEgRNA sequence is selected from any one of SEQ ID NOs: 219-322 or 3592-3603. In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In one aspect, provided herein is a prime editing system comprising: (a) the PEgRNA or the nucleic acid of any one of the aspects or embodiments herein, and (b) a ngRNA, or a nucleic acid encoding the ngRNA, wherein the ngRNA comprises: (i) a spacer comprising at its 3′ end a sequence corresponding to nucleotides 4-20 of any one of SEQ ID NOs: 4 or 195-218; and (ii) an ngRNA core capable of binding a Cas9 protein. In some embodiments, the spacer of the ngRNA comprises at its 3′ end any one of SEQ ID NOs: 4 or 195-218. In some embodiments, the ngRNA core comprises the same sequence as the gRNA core. In some embodiments, the ngRNA core comprises SEQ ID NO: 3666. In some embodiments, the ngRNA comprises any one of SEQ ID NOs: 323-343 or 3604-3610. In some embodiments, the PEgRNA and the ngRNA further comprise 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA and the ngRNA further comprise 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the prime editing system further comprises: (c) a prime editor comprising a Cas9 nickase comprising a nuclease inactivating mutation in a HNH domain, or a nucleic acid encoding the Cas9 nickase, and a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a nucleic acid encoding the N-terminal extein; and (d) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the nucleic acid encoding the Cas9 nickase, and/or the nucleic acid encoding the reverse transcriptase are mRNA.

In one aspect, provided herein is a prime editing guide RNA (PEgRNA) or a nucleic acid encoding the PEgRNA, wherein the PEgRNA comprises: a) a spacer that is complementary to a search target sequence on a first strand of a FANCC gene wherein the spacer comprises at its 3′ end SEQ ID NO: 915; b) a gRNA core capable of binding to a Cas9 protein; and c) an extension arm comprising: i) an editing template that comprises a region of complementarity to an editing target sequence on a second strand of the FANCC gene, and ii) a primer binding site (PBS) that comprises at its 5′ end a sequence that is a reverse complement of nucleotides 10-14 of SEQ ID NO: 915, wherein the first strand and second strand are complementary to each other, wherein the editing target sequence on the second strand comprises or is complementary to a portion of the FANCC gene comprising a c.67del mutation, and wherein the editing template encodes an insertion of a guanine nucleotide at a site corresponding to the c.67del mutation compared to the editing target sequence. In some embodiments, the spacer comprises at its 3′ end any one of SEQ ID NOs: 916, 917, 35, 918, or 919. In some embodiments, the spacer comprises at its 3′ end SEQ ID NO: 35. In some embodiments, the editing template comprises at its 3′ end any one of SEQ ID NOs: 936-964 or 384. In some embodiments, the editing template is 10 to 40 nucleotides in length. In some embodiments, the editing template is 11 to 20 nucleotides in length. In some embodiments, the PBS comprises at its 5′end a sequence corresponding to sequence number 920. In some embodiments, the PBS comprises a sequence corresponding to any one of sequence numbers 920-924 or any one of SEQ ID Nos: 925-934. In some embodiments, the PBS is 8 to 14 nucleotides in length. In some embodiments, the PBS is 8 to 12 nucleotides in length. In some embodiments, the gRNA core comprises SEQ ID NO: 3666. In some embodiments, a PEgRNA sequence comprises any one of SEQ ID NOs: 965-1024 or 3611-3630. In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In one aspect, provided herein is a prime editing guide RNA (PEgRNA), or a nucleic acid encoding the PEgRNA, wherein the PEgRNA comprises: a) a spacer comprising at its 3′ end SEQ ID NO: 915; b) a gRNA core capable of binding to a Cas9 protein; and c) an extension arm comprising: i) an editing template comprising at its 3′ end SEQ ID NO: 935, and ii) a primer binding site (PBS) comprising at its 5′ end a sequence that is a reverse complement of nucleotides 10-14 of SEQ ID NO: 915. In some embodiments, the spacer comprises at its 3′ end any one of SEQ ID NOs: 916, 917, 35, 918, or 919. In some embodiments, the spacer comprises at its 3′ end SEQ ID NO: 35. In some embodiments, the editing template comprises at its 3′ end any one of SEQ ID NOs: 936-964 or 384. In some embodiments, the editing template is 10 to 40 nucleotides in length. In some embodiments, the editing template is 11 to 20 nucleotides in length. In some embodiments, the PBS comprises at its 5′end a sequence corresponding to sequence number 920. In some embodiments, the PBS comprises a sequence corresponding to any one of sequence numbers 920-924 or any one of SEQ ID Nos: 925-934. In some embodiments, the PBS is 8 to 14 nucleotides in length. In some embodiments, the PBS is 8 to 12 nucleotides in length. In some embodiments, the gRNA core comprises SEQ ID NO: 3666. In some embodiments, a PEgRNA sequence comprises any one of SEQ ID NOs: 965-1024 or 3611-3630. In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In one aspect, provided herein is a prime editing system comprising: (a) the PEgRNA or the nucleic acid of any one of the aspects or embodiments herein, and (b) a ngRNA, or a nucleic acid encoding the ngRNA, wherein the ngRNA comprises: (i) a spacer comprising at its 3′ end a sequence corresponding to nucleotides 4-20 of any one of SEQ ID NOs: 4, 195-197, 199-206, 208-218, or 384; and (ii) an ngRNA core capable of binding a Cas9 protein. In some embodiments, the spacer of the ngRNA comprises at its 3′ end any one of SEQ ID NOs: 4, 195-197, 199-206, 208-218, or 384. In some embodiments, the ngRNA core comprises the same sequence as the gRNA core. In some embodiments, the ngRNA core comprises SEQ ID NO: 3666. In some embodiments, the ngRNA comprises any one of SEQ ID NOs: 323-343 or 3604-3610. In some embodiments, the PEgRNA and the ngRNA further comprise 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA and the ngRNA further comprise 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the prime editing system further comprises: (c) a prime editor comprising a Cas9 nickase comprising a nuclease inactivating mutation in a HNH domain, or a nucleic acid encoding the Cas9 nickase, and a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a nucleic acid encoding the N-terminal extein; and (d) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the nucleic acid encoding the Cas9 nickase, and/or the nucleic acid encoding the reverse transcriptase are mRNA.

In one aspect, provided herein is a prime editing guide RNA (PEgRNA) or a nucleic acid encoding the PEgRNA, wherein the PEgRNA comprises: a) a spacer that is complementary to a search target sequence on a first strand of a FANCC gene wherein the spacer comprises at its 3′ end SEQ ID NO: 3086; b) a gRNA core capable of binding to a Cas9 protein; and c) an extension arm comprising: i) an editing template that comprises a region of complementarity to an editing target sequence on a second strand of the FANCC gene, and ii) a primer binding site (PBS) that comprises at its 5′ end a sequence that is a reverse complement of nucleotides 10-14 of SEQ ID NO: 3086, wherein the first strand and second strand are complementary to each other, wherein the editing target sequence on the second strand comprises or is complementary to a portion of the FANCC gene comprising a c.456+4A->T substitution, and wherein the editing template encodes a T to A nucleotide substitution at a site corresponding to the c.456+4A->T substitution compared to the editing target sequence. In some embodiments, the spacer comprises at its 3′ end any one of SEQ ID NOs: 3087, 3088, 2619, 3089, or 3090. In some embodiments, the spacer comprises at its 3′ end SEQ ID NO: 2619. In some embodiments, the editing template comprises SEQ ID NO: 3106 at its 3′ end. In some embodiments, the editing template comprises at its 3′ end any one of SEQ ID NOs: 3107-3123. In some embodiments, the editing template is 10 to 40 nucleotides in length. In some embodiments, the editing template is 27 to 33 nucleotides in length. In some embodiments, the PBS comprises at its 5′ end a sequence corresponding to sequence number 3091. In some embodiments, the PBS comprises a sequence corresponding to any one of sequence numbers 3091-3095 or any one of SEQ ID Nos: 3096-3105. In some embodiments, the PBS is 8 to 14 nucleotides in length. In some embodiments, the gRNA core comprises SEQ ID NO: 3666. In some embodiments, a PEgRNA sequence comprises any one of SEQ ID NOs: 3124-3155. In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In one aspect, provided herein is a prime editing guide RNA (PEgRNA), or a nucleic acid encoding the PEgRNA, wherein the PEgRNA comprises: a) a spacer comprising at its 3′ end SEQ ID NO: 3086; b) a gRNA core capable of binding to a Cas9 protein; and c) an extension arm comprising: i) an editing template comprising at its 3′ end SEQ ID NO: 3106, and ii) a primer binding site (PBS) comprising at its 5′ end a sequence that is a reverse complement of nucleotides 10-14 of SEQ ID NO: 3086. In some embodiments, the spacer comprises at its 3′ end any one of SEQ ID NOs: 3087, 3088, 2619, 3089, or 3090. In some embodiments, the spacer comprises at its 3′ end SEQ ID NO: 2619. In some embodiments, the editing template comprises SEQ ID NO: 3106 at its 3′ end. In some embodiments, the editing template comprises at its 3′ end any one of SEQ ID NOs: 3107-3123. In some embodiments, the editing template is 10 to 40 nucleotides in length. In some embodiments, the editing template is 27 to 33 nucleotides in length. In some embodiments, the PBS comprises at its 5′ end a sequence corresponding to sequence number 3091. In some embodiments, the PBS comprises a sequence corresponding to any one of sequence numbers 3091-3095 or any one of SEQ ID Nos: 3096-3105. In some embodiments, the PBS is 8 to 14 nucleotides in length. In some embodiments, the gRNA core comprises SEQ ID NO: 3666. In some embodiments, a PEgRNA sequence comprises any one of SEQ ID NOs: 3124-3155. In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In one aspect, provided herein is a prime editing system comprising: (a) the PEgRNA or the nucleic acid encoding the PEgRNA of any one of the aspects or embodiments herein, and (b) a ngRNA, or a nucleic acid encoding the ngRNA, wherein the ngRNA comprises: (i) a spacer comprising at its 3′ end a sequence corresponding to nucleotides 4-20 of any one of SEQ ID NOs: 2559, 2561, 2569, 2573, or 2885-2896; and (ii) an ngRNA core capable of binding a Cas9 protein. In some embodiments, the spacer of the ngRNA comprises at its 3′ end any one of SEQ ID NOs: 2559, 2561, 2569, 2573, or 2885-2896. In some embodiments, the ngRNA core comprises the same sequence as the gRNA core. In some embodiments, the ngRNA core comprises SEQ ID NO: 3666. In some embodiments, the ngRNA comprises any one of SEQ ID NOs: 2897-2900. In some embodiments, the PEgRNA and the ngRNA further comprise 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA and the ngRNA further comprise 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the prime editing system further comprises: (c) a prime editor comprising a Cas9 nickase comprising a nuclease inactivating mutation in a HNH domain, or a nucleic acid encoding the Cas9 nickase, and a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a nucleic acid encoding the N-terminal extein; and (d) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the nucleic acid encoding the Cas9 nickase, and/or the nucleic acid encoding the reverse transcriptase are mRNA.

In some embodiments, the spacer, the gRNA core, the editing template, and the PBS form a contiguous sequence in a single molecule. In some embodiments, the PEgRNA comprises from 5′ to 3′, the spacer, the gRNA core, the editing template, and the PBS. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

In some embodiments, the PEgRNA and the ngRNA further comprise 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA and the ngRNA further comprise 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the prime editing system further comprises: (c) a prime editor comprising a Cas9 nickase comprising a nuclease inactivating mutation in a HNH domain, or a nucleic acid encoding the Cas9 nickase, and a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a nucleic acid encoding the N-terminal extein; and (d) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the nucleic acid encoding the Cas9 nickase, and/or the nucleic acid encoding the reverse transcriptase are mRNA.

In one aspect, provided herein is a prime editing system comprising: (a) the PEgRNA of any one of the aspects or embodiments herein, or the nucleic acid encoding the PEgRNA; and (b) a prime editor comprising a Cas9 nickase comprising a nuclease inactivating mutation in the HNH domain, or a nucleic acid encoding the Cas9 nickase, and a reverse transcriptase, or a nucleic acid encoding the reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the nucleic acid encoding the Cas9 nickase, and/or the nucleic acid encoding the reverse transcriptase are mRNA.

In one aspect, provided herein is a prime editing system comprising: (a) the PEgRNA of any one of the aspects or embodiments herein, or the nucleic acid encoding the PEgRNA; (b) an N-terminal extein comprising an N-terminal fragment of a prime editor fusion protein and an N-intein or a nucleic acid encoding the N-terminal extein; and (c) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment. In some embodiments, the nucleic acid encoding the Cas9 nickase, and/or the nucleic acid encoding the reverse transcriptase are mRNA.

In one aspect, provided herein is a population of viral particles collectively comprising the one or more nucleic acids encoding the prime editing system of any one of the aspects or embodiments herein. In some embodiments, the viral particles are AAV particles.

In one aspect, provided herein is a LNP comprising the prime editing system of any one of the aspects or embodiments herein. In some embodiments, the PEgRNA, the nucleic acid encoding the Cas9 nickase, and the nucleic acid encoding the reverse transcriptase. In some embodiments, the nucleic acid encoding the Cas9 nickase and the nucleic acid encoding the reverse transcriptase are mRNA. In some embodiments, the nucleic acid encoding the Cas9 nickase and the nucleic acid encoding the reverse transcriptase are the same molecule.

In one aspect, provided herein is a method of correcting or editing a FANCC gene, the method comprising contacting the FANCC gene with: (a) the PEgRNA of any one of the aspects or embodiments herein and a prime editor comprising a Cas9 nickase comprising a nuclease inactivating mutation in a HNH domain and a reverse transcriptase, (b) the prime editing system of any one of the aspects or embodiments herein, (c) the population of viral particles of any one of the aspects or embodiments herein, or (d) the LNP of any one of the aspects or embodiments herein. In some embodiments, the FANCC gene is in a cell. In some embodiments, the FANCC gene comprises a mutation relative to a corresponding wild-type FANCC gene. In some embodiments, the mutation is c.67del or c.456+4A->T. In some embodiments, the cell is a mammalian cell. In some embodiments, the cell is a human cell. In some embodiments, the cell is a primary cell. In some embodiments, the cell is a hematopoietic stem cell or a hematopoietic pluripotent stem cell. In some embodiments, the cell is in a subject. In some embodiments, the subject is a human. In some embodiments, the cell is from a subject having Fanconi Anemia. In some embodiments, the cell is an allogeneic cell.

In one aspect, provided herein is a cell generated by the method of any one of the aspects or embodiments herein.

In one aspect, provided herein is a population of cells generated by the method of any one of the aspects or embodiments herein.

In one aspect, provided herein is a method for treating Fanconi Anemia in a subject in need thereof, the method comprising administering to the subject: (a) the PEgRNA of any one of the aspects or embodiments herein and a prime editor comprising a Cas9 nickase comprising a nuclease inactivating mutation in a HNH domain and a reverse transcriptase, (b) the prime editing system of any one of the aspects or embodiments herein, (c) the cell of any one of the aspects or embodiments herein, or (d) the population of cells of any one of the aspects or embodiments herein. In some embodiments, the subject is a human.

In one aspect, provided herein is a prime editing guide RNA (PEgRNA) or a nucleic acid encoding the PEgRNA comprising: a) a spacer comprising at its 3′ end a PEgRNA spacer sequence selected from any one of Tables 1-52; b) a gRNA core capable of binding to a Cas9 protein, and c) an extension arm comprising: i) an editing template comprising at its 3′ end an RTT sequence selected from the same Table as the PEgRNA Spacer sequence, and ii) a primer binding site (PBS) comprising at its 5′ end a PBS sequence selected from the same Table as the PEgRNA Spacer sequence. In some embodiments, the spacer of the PEgRNA is from 17 to 22 nucleotides in length. In some embodiments, the spacer of the PEgRNA is 20 nucleotides in length. In some embodiments, the editing template has a length of 40 nucleotides or less. In some embodiments, the editing template has a length of 10 to 32 nucleotides. In some embodiments, the editing template is 12 to 16 nucleotides in length. In some embodiments, the editing template is 11 to 20 nucleotides in length. In some embodiments, the PBS is 8 to 14 nucleotides in length. In some embodiments, the PBS is 11 to 12 nucleotides in length. In some embodiments, the gRNA core comprises SEQ ID NO: 3666. In one aspect, provided herein is a prime editing system comprising: (a) the PEgRNA or the nucleic acid encoding the PEgRNA of any one of the aspects or embodiments herein; and (b) a nick guide RNA (ngRNA), or a nucleic acid encoding the ngRNA, wherein the ngRNA comprises a spacer comprising at its 3′ end nucleotides 4-20 of any ngRNA spacer sequence selected from the same Table as the PEgRNA spacer sequence, and an ngRNA core capable of binding to a Cas9 protein. In some embodiments, the spacer of the ngRNA is 17 to 22 nucleotides in length. In some embodiments, the spacer of the ngRNA comprises at its 3′ end nucleotides 3-20, 2-20, or 1-20 of the ngRNA spacer sequence selected from the same Table as the PEgRNA Spacer sequence. In some embodiments, the spacer of the ngRNA comprises at its 3′ end the ngRNA spacer sequence selected from the same Table as the PEgRNA Spacer sequence. In some embodiments, the ngRNA core comprises the same sequence as the gRNA core. In some embodiments, the ngRNA core comprises SEQ ID NO: 3666. In some embodiments, the prime editing system further comprises: (c) a prime editor comprising a Cas9 nickase comprising a nuclease inactivating mutation in a HNH domain, or a nucleic acid encoding the Cas9 nickase, and a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a nucleic acid encoding the N-terminal extein; and (d) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In one aspect, provided herein is a prime editing system comprising: (a) the PEgRNA of any one of aspects or embodiments herein, or the nucleic acid encoding the PEgRNA; and (b) a prime editor comprising a Cas9 nickase comprising a nuclease inactivating mutation in the HNH domain, or a nucleic acid encoding the Cas9 nickase, and a reverse transcriptase, or a nucleic acid encoding the reverse transcriptase. In one aspect, provided herein is a prime editing system comprising: (a) the PEgRNA of any one of the aspects or embodiments herein, or the nucleic acid encoding the PEgRNA; (b) an N-terminal extein comprising an N-terminal fragment of a prime editor fusion protein and an N-intein or a nucleic acid encoding the N-terminal extein; and (c) a C-terminal extein comprising a C-terminal fragment of the prime editor fusion protein and a C-intein, or a nucleic acid encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase comprising a nuclease inactivating mutation in a HNH domain and a reverse transcriptase. In some embodiments, the Cas9 nickase comprises an amino acid sequence comprising at least 80%, 85%, 90%, 95%, 96%, 97%, 98%, 99%, or 100% sequence identity to SEQ ID NO: 3695 or SEQ ID NO: 3696. In some embodiments, the reverse transcriptase comprises an amino acid sequence comprising at least 80%, 85%, 90%, 95%, 96%, 97%, 98%, 99%, or 100% sequence identity to SEQ ID NO: 3692. In some embodiments, the sequence identities are determined by Needleman-Wunsch alignment of two protein sequences with Gap Costs set to Existence: 11 Extension: 1 where percent identity is calculated by dividing the number of identities by the length of the alignment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of a prime editing guide RNA (PEgRNA) binding to a double stranded target DNA sequence.

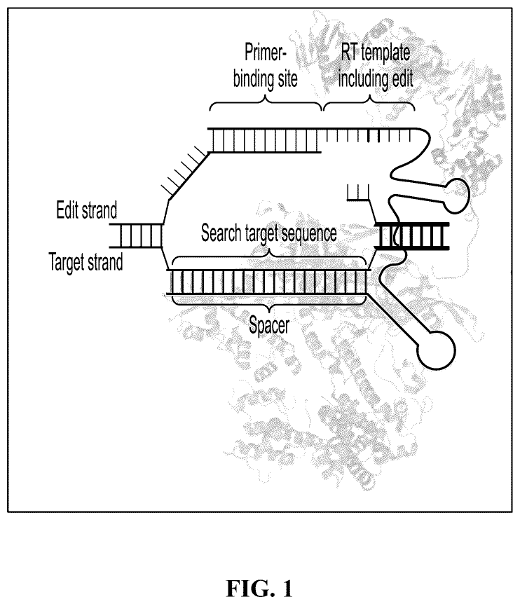

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

### DETAILED DESCRIPTION

Provided herein, in some embodiments, are compositions and methods to edit the target gene FANCC with prime editing. In certain embodiments, provided herein are compositions and methods for correction of mutations in the Fanconi anemia, complementation group C (FANCC) gene associated with Fanconi anemia. Compositions provided herein can comprise prime editors (PEs) that can use engineered guide polynucleotides, e.g., prime editing guide RNAs (PEgRNAs), that can direct PEs to specific DNA targets and can encode DNA edits on the target gene FANCC that serve a variety of functions, including direct correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical means, a range of values that fall within 10% greater than or less than the value. For example, about x means x±(10% * x).

In some embodiments, the cell is a human cell. A cell can be of or derived from different tissues, organs, and/or cell types. In some embodiments, the cell is a primary cell. As used herein, the term “primary cell” means a cell isolated from an organism, e.g., a mammal, which is grown in tissue culture (i.e., in vitro) for the first time before subdivision and transfer to a subculture. In some embodiments, the cell is a stem cell. In some non-limiting examples, mammalian cells, including primary cells and stem cells, can be modified through introduction of one or more polynucleotides, polypeptide, and/or prime editing compositions (e.g., through transfection, transduction, electroporation, and the like) and further passaged. Such modified cells include hematopoietic stem cells (HSCs), hematopoietic stem progenitor cells (HSPC)s, fibroblasts, keratinocytes, epithelial cells (e.g., mammary epithelial cells, intestinal epithelial cells, hepatocytes), endothelial cells, glial cells, neural cells, formed elements of the blood (e.g., lymphocytes, bone marrow cells), muscle cells, precursors of any of these somatic cell types, and stem cells. In some embodiments, the cell is a fibroblast. In some embodiments, the cell is a stem cell. In some embodiments, the cell is a progenitor cell. In some embodiments, the cell is a pluripotent cell (e.g., a pluripotent stem cell) In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is an embryonic stem cell (ESC).

In some embodiments, the cell is a human stem cell. In some embodiments, the cell is a human pluripotent stem cell. In some embodiments, the cell is a human fibroblast. In some embodiments, the cell is an induced human pluripotent stem cell. In some embodiments, the cell is a human stem cell. In some embodiments, the cell is a human embryonic stem cell.

In some embodiments, the cell is a human HSPC (also referred to as a CD34+ cell). In some embodiments, the cell is a hematopoietic stem cell (HSC). In some embodiments, the cell is a hematopoietic progenitor cell (HPC). In some embodiments, hematopoietic stem cells and hematopoietic progenitor cells are referred to as hematopoietic stem or progenitor cells (HSPCs). In some embodiments, the cell is a human HSC. In some embodiments, the cell is a human HPC. In some embodiments, the cell is a human HSPC. In some embodiments, the cell is a long term (LT)-HSC. In some embodiments, the cell is a short-term (ST)-HSC. In some embodiments, the cell is a myeloid progenitor cell. In some embodiments, the cell is a lymphoid progenitor cell. In some embodiments, the cell is a granulocyte monocyte progenitor cell. In some embodiments, the cell is a megakaryocyte erythroid progenitor cell. In some embodiments, the cell is a multipotent progenitor cell (MPP).

In some embodiments, a cell is not isolated from an organism but forms part of a tissue or organ of an organism, e.g., a mammal. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells, lymphoblastoids), precursors of any of these somatic cell types, and stem cells.

In some embodiments, a cell is isolated from an organism. In some embodiments, a cell is derived from an organism. In some embodiments, a cell is a differentiated cell. In some embodiments, the cell is a fibroblast. In some embodiments, the cell is differentiated from an induced pluripotent stem cell. In some embodiments, the cell is differentiated from an HSC or an HPSC. In some embodiments, the cell is differentiated from an induced pluripotent stem cell (iPSC). In some embodiments, the cell is differentiated from an embryonic stem cell (ESC).

In some embodiments, the cell is a differentiated human cell. In some embodiments, cell is a human fibroblast. In some embodiments, the cell is differentiated from an induced human pluripotent stem cell. In some embodiments, the cell is differentiated from a human iPSC or a human ESC.

In some embodiments, the cell comprises a prime editor, a PEgRNA, or a prime editing composition disclosed herein. In some embodiments, the cell is from a human subject. In some embodiments, the human subject has a disease or condition, or is at a risk of developing a disease or a condition associated with a mutation to be corrected by prime editing, for example, Fanconi Anemia. In some embodiments, the cell comprises a mutation associated with Fanconi anemia. In some embodiments, the cell comprises a mutation in a FANCC gene. In some embodiments, the cell is from a human subject, and comprises a prime editor, a PEgRNA, or a prime editing composition for correction of the mutation. In some embodiments, the cell is from the human subject, and the mutation has been edited or corrected by prime editing. In some embodiments, the cell is in a human subject. In some embodiments, the cell comprises a prime editor or a prime editing composition for correction of the mutation. In some embodiments, the cell is in a human subject, and comprises a prime editor or a prime editing composition for correction of the mutation. In some embodiments, the cell is from a human subject. In some embodiments, the cell is from a human subject having Fanconi anemia, wherein the human subject has at least a copy of a FANCC gene that comprises a mutation associated with Fanconi anemia, optionally wherein the human subject has two copies of a FANCC gene, each of which comprises a mutation associated with Fanconi anemia. In some embodiments, the cell comprises at least a copy of, or two copies of, FANCC gene that encodes a functional FANCC protein. In some embodiments, the cell comprises at least a copy, or two copies of, FANCC gene that has the sequence of a wild type FANCC gene. In some embodiments, the cell comprises a prime editor, a PEgRNA, or a prime editing composition. In some embodiments, the mutation in the cell has been edited or corrected by prime editing. In some embodiments, the cell is from the human subject and the mutation has been edited or corrected by prime editing.

The term “substantially” as used herein can refer to a value approaching 100% of a given value. In some embodiments, the term can refer to an amount that can be at least about 70%, 80%, 90%, 91%, 92%, 93%, 94%, 95%, 96%, 97%, 98%, 99%, 99.9%, or 99.99% of a total amount. In some embodiments, the term can refer to an amount that can be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein can be a full-length protein (e.g., a fully processed protein having certain biological function). In some embodiments, a protein can be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor can be a fusion protein comprising a Cas9 protein domain of S. pyogenes and a reverse transcriptase protein domain of a retrovirus (e.g., Moloney murine leukemia virus) or a variant of the retrovirus. A protein that comprises amino acid sequences from different origins or naturally occurring proteins can be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase can encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof can retain one or more of the functions of at least one of the functional domains. For example, a functional fragment of a Cas9 can encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase can comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof can retain one or more of the functions of at least one of the functional domains. For example, in some embodiments, a functional fragment of a Cas9 can comprise one or more amino acid substitutions in a nuclease domain, e.g., a H840A amino acid substitution, compared to the amino acid sequence of a wild type Cas9, but retains the DNA binding ability and lacks the nuclease activity partially or completely.

The term “function” and its grammatical equivalents as used herein refer to a capability of operating, having, or serving an intended purpose. Functional can comprise any percent from baseline to 100% of an intended purpose. For example, functional can comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional can mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence, or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences can exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or a protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs can also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions can be determined in homologous sequences based on alignment, for example, “H840” in a reference Cas9 sequence can correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide can comprise one or more other nucleotide bases, such as inosine (I), which is read by the translation machinery as guanine (G).

In some embodiments, a polynucleotide can be modified. A polynucleotide, e.g., a PEgRNA, can be chemically modified with respect to the A, C, G, T and U nucleotides. In some embodiments, modifications can be on the nucleoside base and/or sugar portion of the nucleosides that comprise the polynucleotide. In some embodiments, the modification can be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides can base pair via hydrogen bonding, which can be Watson Crick, Hoogsteen or reversed Hoogsteen hydrogen bonding. For example, an adenine on one polynucleotide molecule will base pair to a thymine or an uracil on a second polynucleotide molecule and a cytosine on one polynucleotide molecule will base pair to guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity can be a region of 10, 15, 20, 25, 30, 35, 40, 45, 50, or more nucleotides. “Substantial complementary” can also refer to a 100% complementarity over a portion or a region of two polynucleotide molecules. In some embodiments, the portion or the region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression can include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or a nucleic acid sequence of a polynucleotide. Such changes and/or alterations can comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein can refer to a human or a non-human. A subject can be a mammal. A human subject can be male or female. A human subject can be of any age. A subject can be a human embryo. A human subject can be a newborn, an infant, a child, an adolescent, or an adult. A human subject can be in need of treatment for a genetic disease or disorder. In some embodiments, a subject is suffering from, susceptible to, or at a risk of developing FA-C.

The terms “treatment” or “treating” and their grammatical equivalents refer to the medical management of a subject with an intent to cure, ameliorate, or ameliorate a symptom of, a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include supportive treatment, that is, treatment employed to supplement another specific therapy directed toward the improvement of the disease, condition, or disorder. In some embodiments, a condition can be pathological. In some embodiments, a treatment may not completely cure or prevent a disease, condition, or disorder. In some embodiments, a treatment ameliorates, but does not completely cure or prevent a disease, condition, or disorder. In some embodiments, a subject can be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g. a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

### Fanconi Anemia of Complementation Group C (FA-C)

Provided herein are methods and compositions for correcting one or more mutations in a target gene e.g., a FANCC gene. The disclosure also provides methods and compositions for treatment of a subtype of Fanconi anemia; Fanconi anemia group C or FA-C.

“Fanconi Anemia of complementation group C”, “Fanconi Anemia group C”, “FA-C”, or “Fanconi anemia type C” as used in herein refers to an autosomal recessive disease subtype of Fanconi anemia, that is associated with a mutation in a FANCC gene. In some embodiments, the mutation in the FANCC gene results in expression of a mutated FANCC protein. In some embodiments, the mutation in the FANCC gene results in expression of a non-functional FANCC protein. Accordingly, in some embodiments, FA-C refers to a disease subtype of FA that is associated with expression of a mutated FANCC protein.

As used herein, the term “FANCC gene”, “FANCC”, or “FA Complementation Group C gene” is one of a group of classical Fanconi anemia genes whose protein products form a FA core complex. The FANCC gene encodes a FANCC protein. In some embodiments, the FANCC gene is a human FANCC gene. In some embodiments, the FANCC gene (e.g., a human FANCC gene) is localized at positions 95099054 to 95317730 of chromosome 9. In some embodiments, the FANCC gene comprises 14 exons. In some embodiments, the FANCC gene codes an ORF of 1677 bp, the translation of which results in a FANCC protein of 558 aa, weighting about 63 kDa. Exemplary sequence of a wild type FANCC gene is set forth as SEQ ID NO: 3809.

In some embodiments, the FANCC gene comprises a mutation relative to a wild type FANCC gene (for example, a wild type FANCC gene set forth as SEQ ID NO: 3809). In some embodiments, the mutation occurs within an intron of the FANCC gene. In some embodiments, the mutation is within an intron, at a splice site, or adjacent to a splice site, of the FANCC gene, and affects the splicing of a transcript encoded by the FANCC gene.

In some embodiments, the mutation in a FANCC gene is located between positions 95,171, 933 and 95,172,133 of human chromosome 9 according to GRCh38. In some embodiments, the mutation comprises a transversion. In some embodiments, the mutation in a FANCC gene is located at position 95,172,033 of human chromosome 9 according to GRCh38. In some embodiments, the mutation is an A to T transversion at position 95,172,033 of human chromosome 9 according to GRCh38, referred to as the “IVS4+4A>T” mutation. In some embodiments, the mutation in a FANCC gene (e.g., a IVS4+4A>T) encodes a mutated FANCC protein that comprises a truncation after the 4th exon. The mutation, IVS4+4A>T, is also referred to as c456+4A>T or IVS4, A-T, +4, and is located at position 95,172,033 of chromosome 9 according to GRCh38.

In some embodiments, the mutation in a FANCC gene is located between positions 95, 248, 125 and 95, 248, 325 of human chromosome 9 according to GRCh38. In some embodiments, the FANCC gene comprises a mutation in an exon relative to a wild type FANCC gene (for example, a wild type FANCC gene set forth as SEQ ID NO 3809). In some embodiments, the mutation comprises a single G nucleotide deletion at position 95,248,225 of human chromosome 9 according to GRCh38, referred to as the “322delG” mutation. The mutation is also referred to as c.67del. In some embodiments, the mutation (e.g., c.67del or 322delG) results in a frameshift mutation in the protein sequence. In some embodiments, the mutation in a FANCC gene (e.g., c.67del or 322delG) results in a premature stop codon. In some embodiments, a target FANCC gene is a FANCC gene that is a target gene for prime editing using the compositions and methods disclosed herein.

The term “FANCC protein” or “FANCC” as used herein refers to a protein encoded by the FANCC gene. In some embodiments, FANCC is a human FANCC. Amino acid sequences of a wild type FANCC are known in the art and available publicly, for example, from the NCBI website. Non-limiting example includes NCBI accession number NP_000127, which is incorporated herein in its entirety. FANCC protein can be used interchangeably with Fanconi Anemia Complementation Group C, FACC, FAC, or Fanconi Anemia Group C Protein. FANCC is one of the group of proteins that form a FA core complex. The FANCC protein complexes with at least three other proteins FANCA, FANCF, and FANCG to form the FA core complex. The FA core complex including the FANCC protein, functions in DNA damage repair in part by post-translation modification of a FANCD2 protein.

FA-C patients with the c.456+4A->T mutation (also referred to as the IVS4+4A->T mutation) demonstrate higher incidence and more rapid emergence of BMF compared to other groups at a median age of 2.7 years. Patients with exon 1 c.67del (322delG) have more typical disease progression with hematologic symptoms at a median age of 7 years.

BMF of FA-C patients is attributable to impaired CD34+ hematopoietic stem cell pool. FA-C patients develop progressive bone marrow failure during childhood and require an allogeneic related or unrelated HSCT. Clinical presentation of FA-C includes congenital anomalies, pan cytopenia, BMF, and a predisposition to myeloid and epithelial cancers. Cytopenias and BMF affect the majority of FA-C patients within the first decade of life. The proposed mechanism for cytopenias and BMF is intolerance to oxidative stress in response to accumulated DNA damage in the CD34+ HSPC pool.

Rapid diagnostic testing is achieved by exposure of patient peripheral blood (PB) cells to DNA crosslinking agents such as diepoxybutane (DEB) or mitomycin C (MMC), which results in high frequency characteristic chromosomal breaks. Patients would need genotyping test after this diagnosis to identify predominant FANCC mutations. Of note, FA-C patients with transversion mutation c.456+4A->T (IVS4+4A>T) will present very early in life (median 2.7 years of age). Corrected cells should exhibit a survival and proliferative advantage over endogenous FA cells. Biomarkers for correction include rescue from sensitivity to MMC and DEB in bone marrow CD34+ HSPCs and peripheral blood lymphocytes. Provided herein are compositions and methods to correct a mutation in a FANCC gene (e.g., c.456+4A->T (IVS4+4A>T), or c.67del (322delG)) by prime editing. Provided herein are compositions and methods to install a nucleotide change in a FANCC gene.

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target gene of prime editing can comprise a double stranded DNA molecule having two complementary strands: a first strand that can be referred to as a “target strand” or a “non-edit strand”, and a second strand that can be referred to as a “non-target strand,” or an “edit strand.” In some embodiments, in a prime editing guide RNA (PEgRNA), a spacer sequence is complementary or substantially complementary to a specific sequence on the target strand, which can be referred to as a “search target sequence”. In some embodiments, the spacer sequence anneals with the target strand at the search target sequence. The target strand can also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence can have a substantially identical sequence as the protospacer sequence on the edit strand of a target gene (e.g. FANCC gene), except that the spacer sequence can comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase that comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and which encodes a single strand of DNA. The editing template may comprise a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit position(s). As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template may encode the wild-type or non-disease associated gene sequence (or its complement if the edit strand is the antisense strand of a gene). In some embodiments, the editing template may encode the wild-type or non-disease associated protein but contain one or more synonymous mutations relative to the wild-type or non-disease associated protein coding region. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer).

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template can be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions. In some embodiments, the editing template comprises at least 4 contiguous nucleotides of complementarity with the edit strand wherein the at least 4 nucleotides contiguous are located upstream of the 5′ most edit in the editing template.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having a 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor can be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor can comprise a S. pyogenes Cas9 polypeptide and a Moloney murine leukemia virus (M-MLV) reverse transcriptase polypeptide.

In some embodiments, polypeptide domains of a prime editor can be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor can comprise a DNA binding domain and a reverse transcriptase domain associated with each other by an RNA-protein recruitment aptamer, e.g. a MS2 aptamer, which can be linked to a PEgRNA. Prime editor polypeptide components can be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein can comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g. a DNA polymerase domain. The DNA polymerase domain can be a wild-type DNA polymerase domain, a full-length DNA polymerase protein domain, or can be a functional mutant, a functional variant, or a functional fragment thereof. In some embodiments, the polymerase domain is a template dependent polymerase domain. For example, the DNA polymerase can rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA can comprise an RNA portion (including the spacer and the gRNA core) and a DNA portion (the extension arm comprising the editing template that includes a strand of DNA).

In some embodiments, the DNA polymerases can be wild type polymerases from eukaryotic, prokaryotic, archaeal, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a RevI DNA polymerase. In some embodiments, the DNA polymerase is a human RevI DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain can be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV RT, MMLVRT or M-MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT, a functional mutant, a functional variant, or a functional fragment thereof.

In some embodiments, the prime editor comprises a reference M-MLV RT, a functional mutant, a functional variant, or a functional fragment thereof. In some embodiments, the RT domain or a RT is a M-MLV RT (e.g., wild-type M-MLV RT, a functional mutant, a functional variant, or a functional fragment thereof). In some embodiments, the RT domain or a RT is a M-MLV RT (e.g., a reference M-MLV RT, a functional mutant, a functional variant, or a functional fragment thereof). In some embodiments, a M-MLV RT comprises an amino acid sequence as set forth in any one of SEQ ID NOs: 3690-3692. In some embodiments, the prime editor comprises a wild type M-MLV RT. An exemplary amino acid sequence of a wild type M-MLV RT is provided in SEQ ID NO: 3691.

(SEQ ID NO: 3691)
TLNIEDEHRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLIENSSP.

In some embodiments, the prime editor comprises a reference M-MLV RT. An exemplary amino acid sequence of a reference M-MLV RT is provided in SEQ ID NO: 3690.

(SEQ ID NO: 3690)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLIENSSP

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 3690, where X is any amino acid other than the original amino acid in the reference M-MLV RT. In some embodiments, the prime editor comprises a M-MMLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO: 3690. In some embodiments, the prime editor comprises a M-MLV RT comprising one or more amino acid substitutions D200N, T330P, L603W, T306K, and W313F as compared to the reference M-MLV RT as set forth in SEQ ID NO: 3690. In some embodiments, the prime editor comprises a M-MLV RT comprising amino acid substitutions D200N, T330P, L603W, T306K, and W313F as compared to a reference M-MLV RT as set forth in SEQ ID NO: 3690. In some embodiments, the prime editor comprises a M-MLV RT comprising one or more of amino acid substitutions D200N, T330P, L603W, T306K, and W313F as compared to a wild type M-MMLV RT as set forth in SEQ ID NO: 3691. In some embodiments, a prime editor may comprise amino acid substitutions D200N, T330P, L603W, T306K, and W313F as compared to a reference M-MLV RT as set forth in SEQ ID NO: 3690. In some embodiments, the prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 3690, 3691, or 3692. In some embodiments, the prime editor comprises a M-MLV RT that comprises an amino acid sequence that is selected from the group consisting of SEQ ID NOs: 3690, 3691, and 3692, or a variant or fragment thereof.

In some embodiments, the prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in SEQ ID NO: 3692. In some embodiments, the prime editor comprises a M-MLV RT that comprises an amino acid sequence set forth in SEQ ID NO: 3692.

(SEQ ID NO: 3692)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFNEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGWLTSEGKEIKNKDEILALLKALFLPKRLSIIHCPG
HQKGHSAEARGNRMADQAARKAAITETPDTSTLLIENSSP.

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a wild type RT, e.g SEQ ID NO: 3691. In some embodiments, the RT variant comprises a fragment of a wild type RT, e.g., SEQ ID NO: 3691, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the wild type RT, e.g., SEQ ID NO: 3691. In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding wild type RT (M-MLV reverse transcriptase) (e.g., SEQ ID NO: 3691).

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., SEQ ID NO: 3690. In some embodiments, the RT variant comprises a fragment of a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of a reference RT, e.g., SEQ ID NO: 3690. In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a reference RT, e.g., a M-MLV RT, e.g., SEQ ID NO: 3690. In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the functional RT variant, e.g., a functional MMLV RT variant, is truncated at the C-terminus to abolish or reduce RNAase H activity and still retain DNA polymerase activity.

In some embodiments, a prime editing composition or a prime editing system disclosed herein comprises a polynucleotide (e.g., a DNA, a RNA, e.g., a mRNA) that encodes a M-MLV RT. In some embodiments, the polynucleotide encodes a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of the SEQ ID NOs: 3690, 3691, or 3692. In some embodiments, the polynucleotide encodes a M-MLV RT that comprises an amino acid sequence that is selected from the group consisting of SEQ ID NOs: 3690, 3691, and 3692. In some embodiments, the polynucleotide encodes a M-MLV RT that comprises an amino acid sequence that is set forth in SEQ ID NO: 3692.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT. In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 3693-3720. In some embodiments, the DNA-binding domain of a prime editor is a programmable DNA binding domain. In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in Table 53. In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in SEQ ID NOs: 3693-3720. In some embodiments, the DNA binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g. a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprises a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA binding domain of a prime editor comprises a nuclease having full nuclease activity. In some embodiments, the DNA binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has a nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), Cas 4D, and homologues, modified or engineered variants, mutants, and/or functional fragments thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

Non-limiting examples of suitable organism include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis).

In some embodiments, a Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or functional fragment of a wild type Cas protein. In some embodiments, a Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, can comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g. nickase) or abolished nuclease activity while maintaining its ability to target a nucleic acid locus at a search target sequence when complexed with a guide nucleic acid, e.g. a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain e.g., an amino acid substitution that reduces or abolishes nuclease activity of the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain e.g., an amino acid substitution that reduces or abolishes nuclease activity of the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some embodiments, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein can comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide, e.g., comprising an amino acid sequence as set forth in NCBI Accession No. WP_038431314 or a fragment or variant thereof. In some embodiments, a Cas9 polypeptide is a SaCas9 polypeptide, e.g., comprising an amino acid sequence as set forth in Uniprot Accession No. J7RUA5 or a fragment or variant thereof. In some embodiments, a Cas9 polypeptide is a ScCas9 polypeptide, e.g., comprising an amino acid sequence as set forth in Uniprot Accession No. AOA3P5YA78 or a fragment or variant thereof. In some embodiments, a Cas9 polypeptide is a StCas9 polypeptide, e.g., comprising an amino acid sequence as set forth in NCBI Accession No. WP_007896501.1 or a fragment or variant thereof. In some embodiments, a Cas9 polypeptide is a SluCas9 polypeptide, e.g., comprising an amino acid sequence as set forth in any of NCBI Accession No. WP_230580236.1 or WP_250638315.1 or WP_242234150.1, WP_241435384.1, WP_002460848.1, KAK58371.1, or a fragment or variant thereof. In some embodiments, a Cas9 polypeptide is a NmCas9 polypeptide, e.g., comprising an amino acid sequence as set forth in any of NCBI Accession No. WP_002238326.1 or WP_061704949.1 or a fragment or variant thereof. In some embodiments, a Cas9 polypeptide is a CjCas9 polypeptide, e.g., comprising an amino acid sequence as set forth in any of NCBI Accession No. WP_100612036.1, WP 116882154.1, WP 116560509.1, WP_116484194.1, WP_116479303.1, WP_115794652.1, WP_100624872.1, or a fragment or variant thereof. In some embodiments, a Cas9 polypeptide is a FnCas9 polypeptide, e.g., comprising the amino acid sequence as set forth in Uniprot Accession No. AOQ5Y3 or a fragment or variant thereof. In some embodiments, a Cas9 polypeptide is a TdCas9 polypeptide, e.g., comprising the amino acid sequence as set forth in NCBI Accession No. WP_147625065.1 or a fragment or variant thereof. In some embodiments, a Cas9 polypeptide is a chimera comprising domains from two or more of the organisms described herein or those known in the art. In some embodiments, a Cas9 polypeptide is a Cas9 polypeptide from Streptococcus macacae, e.g., comprising the amino acid sequence as set forth in NCBI Accession No. WP_003079701.1 or a fragment or variant thereof. In some embodiments, a Cas9 polypeptide is a Cas9 polypeptide generated by replacing a PAM interaction domain of a SpCas9 with that of a Streptococcus macacae Cas9 (Spy-mac Cas9). Exemplary Cas sequences are provided in Table 53 below.

In some embodiments, a Cas9 protein comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 3693-3720. In some embodiments, a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 3694, 3695, 3696, 3698, 3699, 3701, 3702, 3704, 3705, 3707, 3708, 3710, 3711, 3713, 3714, 3716, 3717, 3719, or 3720. In some embodiments, a Cas9 protein comprises an amino acid sequence that is selected from the group consisting of SEQ ID NOs: 3693-3720. In some embodiments, a prime editor comprises a Cas9 protein that comprises an amino acid sequence that lacks a N-terminus methionine relative to an amino acid sequence set forth in any one of SEQ ID NOs: 3693, 3694, 3697, 3698, 3700, 3701, 3703, 3704, 3706, 3707, 3709, 3710, 3712, 3713, 3715, 3716, 3718, or 3719. In some embodiments, the prime editing compositions or prime editing systems disclosed herein comprises a polynucleotide (e.g., a DNA, or an RNA, e.g., an mRNA) that encodes a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 3693-3720.

In some embodiments, a Cas9 protein comprises a Cas9 protein from Streptococcus pyogenes (Sp), e.g., as according to NC_002737.2:854751-858857 or the protein encoded by UniProt Q99ZW2, e.g., as according to SEQ ID NO: 3693. In some embodiments, a prime editor comprises a Cas9 protein (e.g., a SpCas9) as according to any one of the sequences set forth in SEQ ID NOs: 3693-3696 or a variant thereof. In some embodiments, the Cas9 protein is a SpCas9. In some embodiments, a SpCas9 can be a wild type SpCas9, a SpCas9 variant, or a nickase SpCas9. In some embodiments, the SpCas9 lacks the N-terminus methionine relative to a corresponding SpCas9 (e.g., a wild type SpCas9, a SpCas9 variant or a nickase SpCas9). In some embodiments, a prime editor comprises a Cas9 protein or a variant thereof not including the N-terminus methionine. In some embodiments, a wild type SpCas9 comprises an amino acid sequence set forth in SEQ ID NO: 3693. In some embodiments, a prime editor comprises a Cas9 protein comprising one or more mutations (e.g., amino acid substitutions, insertions and/or deletions) relative to a corresponding wild type Cas9 protein (e.g., a wild type SpCas9). In some embodiments, the Cas9 protein comprising one or more mutations relative to a wild type Cas9 (e.g., a wild type SpCas9) protein comprises an amino acid sequence set forth in SEQ ID NO: 3694, SEQ ID NO:3695 or SEQ ID NO: 3696. Exemplary Streptococcus pyogenes Cas9 (SpCas9) amino acid sequence useful in the prime editors disclosed herein are provided below in SEQ ID NOs: 3693-3696 or 3703-3708.

In some embodiments, a prime editor comprises a Cas9 protein (e.g., a SluCas9) as according to any one of the SEQ ID NOS: 3697-3699 or a variant thereof. In some embodiments, a prime editor comprises a Cas9 protein from Staphylococcus lugdunensis (SluCas9) e.g., as according to any one of the SEQ ID NOs: 3697-3699 or a variant thereof. In some embodiments, the Cas9 protein is a SluCas9. In some embodiments, a SluCas9 can be a wild type SluCas9, a SluCas9 variant, or a nickase SluCas9. In some embodiments, the SluCas9 lacks the N-terminus methionine relative to a corresponding SluCas9 (e.g., a wild type SluCas9, a SluCas9 variant or a nickase SluCas9). In some embodiments, a wild type SluCas9 comprises an amino acid sequence set forth in SEQ ID NO: 3697. In some embodiments, a prime editor comprises a Cas9 protein comprising one or more mutations (e.g., amino acid substitutions, insertions and/or deletions) relative to a corresponding wild type Cas9 protein (e.g., a wild type SluCas9). In some embodiments, the Cas9 protein comprising one or mutations relative to a wild type Cas9 protein comprises an amino acid sequence set forth in SEQ ID NO: 3698 or SEQ ID NO: 3699. Exemplary Staphylococcus lugdunensis Cas9 (SluCas9) amino acid sequence useful in the prime editors disclosed herein are provided below in SEQ ID NOs: 3697-3699.

In some embodiments, a prime editor comprises a Cas9 protein from Staphylococcus aureus (SaCas9) e.g., as according to any of the SEQ ID NOS: 3700-3702, or a variant thereof. In some embodiments, a prime editor comprises a Cas9 protein from Staphylococcus aureus (SaCas9) e.g., as according to any one of the SEQ ID NOS: 3700-3702, or a variant thereof. In some embodiments, the Cas9 protein is a SaCas9. In some embodiments, a SaCas9 can be a wild type SaCas9, a SaCas9 variant, or a nickase SaCas9. In some embodiments, the SaCas9 lacks the N-terminus methionine relative to a corresponding SaCas9 (e.g., a wild type SaCas9, a SaCas9 variant or a nickase SaCas9). In some embodiments, a wild type SaCas9 comprises an amino acid sequence set forth in SEQ ID NO: 3700. In some embodiments, a prime editor comprises a Cas9 protein comprising one or more mutations (e.g., amino acid substitutions, insertions and/or deletions relative to a corresponding wild type Cas9 protein (e.g., a wild type SaCas9). In some embodiments, the Cas9 protein comprising one or more mutations relative to a wild type Cas9 protein comprises an amino acid sequence set forth in SEQ ID NO: 3701 or SEQ ID NO: 3702. Exemplary Staphylococcus aureus Cas9 (SaCas9) amino acid sequence useful in the prime editors disclosed herein are provided below in SEQ ID NOs: 3700-3702.

In some embodiments, a prime editor comprises a Cas protein, e.g., a Cas9 variant, comprising modifications that allow altered PAM recognition. Exemplary Cas9 protein amino acid sequence (e.g., Cas9 variant with altered PAM recognition specificities) that are useful in the Prime editors of the disclosure are provided below in SEQ ID NOs: 3703-3711, 3718-3720. In some embodiments, a prime editor comprises a Cas9 protein as according to any one of the sequences set forth in SEQ ID NOs: 3703-3711, 3718-3720 or a variant thereof. In some embodiments, the Cas9 protein is a Cas9 variant, for example, a SpCas9 variant (e.g., SpCas9-NG, SpCas9-NGA, SpRY, or SpG). In some embodiments, the Cas9 protein lacks the N-terminus methionine relative to a corresponding Cas9 protein (e.g., a Cas9 variant set forth in any one of SEQ ID NOs: 3703, 3704, 3706, 3707, 3709, 3710, 3718, or 3719). In some embodiments, a prime editor comprises a Cas9 protein (e.g., a Cas9 variant), having an amino acid sequence as according to any one of SEQ ID NOs: 3703, 3704, 3706, 3707, 3709, 3710, 3718, or 3719 not including the N-terminus methionine. In some embodiments, a prime editor comprises a Cas9 protein comprising one or more mutations (e.g., amino acid substitutions, insertions and/or deletions) relative to a corresponding Cas9 protein (e.g., a Cas9 protein set forth in any one of SEQ ID NOs: 3703, 3706, 3709, or 3718). In some embodiments, the Cas9 protein comprising one or mutations relative to a corresponding Cas9 protein comprises an amino acid sequence set forth in any one of SEQ ID NOs: 3704, 3705, 3707, 3708, 3710, 3711, 3719, or 3720.

In some embodiments, a Cas9 protein is a chimeric Cas9, e.g., modified Cas9, e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified sluCas9 shows increased editing efficiency and/or specificity relative to a sluCas9 that is not modified. In some embodiments, a Cas9, e.g., a sRGN shows ability to cleave a 5′-NNGG-3′ PAM-containing target. In some embodiments, a prime editor comprises a Cas9 protein (e.g., a chimeric Cas9), e.g., as according any one of the sequences set forth in SEQ ID NOs: 3712-3717, or a variant thereof. Exemplary amino acid sequences of Cas9 protein (e.g., sRGN) useful in the prime editors disclosed herein are provided below in SEQ ID NOs: 3712-3717. In some embodiments, a prime editor comprises a Cas9 protein, that lacks a N-terminus methionine relative to SEQ ID NO: 3712, 3713, 3715, or 3716. In some embodiments, a prime editor comprises a Cas9 protein comprising one or more mutations (e.g., amino acid substitutions, insertions and/or deletions) relative to a corresponding Cas9 protein (e.g., a Cas9 protein set forth in SEQ ID NO: 3712 or SEQ ID NO: 3715). In some embodiments, the Cas9 protein comprising one or mutations relative to a corresponding Cas9 protein comprises an amino acid sequence set forth in any one of SEQ ID NOs: 3713, 3714, 3716, or 3717.

TABLE 53
Exemplary Cas protein sequences

| SEQ | | |
|---|---|---|
| ID | Sequence | |
| NO: | description | Amino acid sequence |
| 3693 | wild type | MDKKYSIGLDIGTNSVGWAVITDEYKVPSKKFKVLGNTDRHSIKKNLIGALLFDSGETAEATRL |
| | Streptococcus | KRTARRRYTRRKNRICYLQEIFSNEMAKVDDSFFHRLEESFLVEEDKKHERHPIFGNIVDEVAY |
| | Pyogenes | HEKYPTIYHLRKKLVDSTDKADLRLIYLALAHMIKFRGHFLIEGDLNPDNSDVDKLFIQLVQTYN |
| | Cas9 (SpCas9) | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFDSPTVAYSVLVVA |
| | | KVEKGKSKKLKSVKELLGITIMERSSFEKNPIDFLEAKGYKEVKKDLIIKLPKYSLFELENGRKRM |
| | | LASAGELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKRYTSTKEVL |
| | | DATLIHQSITGLYETRIDLSQLGGD |
| 3694 | SpCas9 | MDKKYSIGLDIGTNSVGWAVITDEYKVPSKKFKVLGNTDRHSIKKNLIGALLFDSGETAEATRL |
| | H840A | KRTARRRYTRRKNRICYLQEIFSNEMAKVDDSFFHRLEESFLVEEDKKHERHPIFGNIVDEVAY |
| | nickase | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFDSPTVAYSVLVVA |
| | | KVEKGKSKKLKSVKELLGITIMERSSFEKNPIDFLEAKGYKEVKKDLIIKLPKYSLFELENGRKRM |
| | | LASAGELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKRYTSTKEVL |
| | | DATLIHQSITGLYETRIDLSQLGGD |
| 3695 | Met(−) | DKKYSIGLDIGTNSVGWAVITDEYKVPSKKFKVLGNTDRHSIKKNLIGALLFDSGETAEATRLK |
| | SpCas9 | RTARRRYTRRKNRICYLQEIFSNEMAKVDDSFFHRLEESFLVEEDKKHERHPIFGNIVDEVAYH |
| | H840A | EKYPTIYHLRKKLVDSTDKADLRLIYLALAHMIKFRGHFLIEGDLNPDNSDVDKLFIQLVQTYN |
| | nickase | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFDSPTVAYSVLVVA |
| | | KVEKGKSKKLKSVKELLGITIMERSSFEKNPIDFLEAKGYKEVKKDLIIKLPKYSLFELENGRKRM |
| | | LASAGELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKRYTSTKEVL |
| | | DATLIHQSITGLYETRIDLSQLGGD |
| 3696 | Met(−) CAS9 | DKKYSIGLDIGTNSVGWAVITDEYKVPSKKFKVLGNTDRHSIKKNLIGALLFDSGETAEATRLK |
| | (R221K N394K | RTARRRYTRRKNRICYLQEIFSNEMAKVDDSFFHRLEESFLVEEDKKHERHPIFGNIVDEVAYH |
| | H840A) | EKYPTIYHLRKKLVDSTDKADLRLIYLALAHMIKFRGHFLIEGDLNPDNSDVDKLFIQLVQTYN |
| | nickase | QLFEENPINASGVDAKAILSARLSKSRKLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLKREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFDSPTVAYSVLVVA |
| | | KVEKGKSKKLKSVKELLGITIMERSSFEKNPIDFLEAKGYKEVKKDLIIKLPKYSLFELENGRKRM |
| | | LASAGELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKRYTSTKEVL |
| | | DATLIHQSITGLYETRIDLSQLGGD |
| 3697 | wild type | MNQKFILGLDIGITSVGYGLIDYETKNIIDAGVRLFPEANVENNEGRRSKRGSRRLKRRRIHRLE |
| | Staphylococcus | RVKKLLEDYNLLDQSQIPQSTNPYAIRVKGLSEALSKDELVIALLHIAKRRGIHKIDVIDSNDDV |
| | lugdunensis | GNELSTKEQLNKNSKLLKDKFVCQIQLERMNEGQVRGEKNRFKTADIIKEIIQLLNVQKNFHQ |
| | (Slu)Cas9 | LDENFINKYIELVEMRREYFEGPGKGSPYGWEGDPKAWYETLMGHCTYFPDELRSVKYAYSA |
| | | DLFNALNDLNNLVIQRDGLSKLEYHEKYHIIENVFKQKKKPTLKQIANEINVNPEDIKGYRITKS |
| | | GKPQFTEFKLYHDLKSVLFDQSILENEDVLDQIAEILTIYQDKDSIKSKLTELDILLNEEDKENIAQ |
| | | LTGYTGTHRLSLKCIRLVLEEQWYSSRNQMEIFTHLNIKPKKINLTAANKIPKAMIDEFILSPVV |
| | | KRTFGQAINLINKIIEKYGVPEDIIIELARENNSKDKQKFINEMQKKNENTRKRINEIIGKYGNQ |
| | | NAKRLVEKIRLHDEQEGKCLYSLESIPLEDLLNNPNHYEVDHIIPRSVSFDNSYHNKVLVKQSE |
| | | NSKKSNLTPYQYFNSGKSKLSYNQFKQHILNLSKSQDRISKKKKEYLLEERDINKFEVQKEFINR |
| | | NLVDTRYATRELTNYLKAYFSANNMNVKVKTINGSFTDYLRKVWKFKKERNHGYKHHAEDA |
| | | LIIANADFLFKENKKLKAVNSVLEKPEIESKQLDIQVDSEDNYSEMFIIPKQVQDIKDFRNFKYS |
| | | HRVDKKPNRQLINDTLYSTRKKDNSTYIVQTIKDIYAKDNTTLKKQFDKSPEKFLMYQHDPRT |
| | | FEKLEVIMKQYANEKNPLAKYHEETGEYLTKYSKKNNGPIVKSLKYIGNKLGSHLDVTHQFKSS |
| | | TKKLVKLSIKPYRFDVYLTDKGYKFITISYLDVLKKDNYYYIPEQKYDKLKLGKAIDKNAKFIASFY |
| | | KNDLIKLDGEIYKIIGVNSDTRNMIELDLPDIRYKEYCELNNIKGEPRIKKTIGKKVNSIEKLTTDV |
| | | LGNVFTNTQYTKPQLLFKRGN |
| 3698 | SluCas9 | MNQKFILGLDIGITSVGYGLIDYETKNIIDAGVRLFPEANVENNEGRRSKRGSRRLKRRRIHRLE |
| | N582A | RVKKLLEDYNLLDQSQIPQSTNPYAIRVKGLSEALSKDELVIALLHIAKRRGIHKIDVIDSNDDV |
| | nickase | GNELSTKEQLNKNSKLLKDKFVCQIQLERMNEGQVRGEKNRFKTADIIKEIIQLLNVQKNFHQ |
| | | LDENFINKYIELVEMRREYFEGPGKGSPYGWEGDPKAWYETLMGHCTYFPDELRSVKYAYSA |
| | | DLFNALNDLNNLVIQRDGLSKLEYHEKYHIIENVFKQKKKPTLKQIANEINVNPEDIKGYRITKS |
| | | GKPQFTEFKLYHDLKSVLFDQSILENEDVLDQIAEILTIYQDKDSIKSKLTELDILLNEEDKENIAQ |
| | | LTGYTGTHRLSLKCIRLVLEEQWYSSRNQMEIFTHLNIKPKKINLTAANKIPKAMIDEFILSPVV |
| | | KRTFGQAINLINKIIEKYGVPEDIIIELARENNSKDKQKFINEMQKKNENTRKRINEIIGKYGNQ |
| | | NAKRLVEKIRLHDEQEGKCLYSLESIPLEDLLNNPNHYEVDHIIPRSVSFDNSYHNKVLVKQSE |
| | | ASKKSNLTPYQYFNSGKSKLSYNQFKQHILNLSKSQDRISKKKKEYLLEERDINKFEVQKEFINR |
| | | NLVDTRYATRELTNYLKAYFSANNMNVKVKTINGSFTDYLRKVWKFKKERNHGYKHHAEDA |
| | | LIIANADFLFKENKKLKAVNSVLEKPEIESKQLDIQVDSEDNYSEMFIIPKQVQDIKDFRNFKYS |
| | | HRVDKKPNRQLINDTLYSTRKKDNSTYIVQTIKDIYAKDNTTLKKQFDKSPEKFLMYQHDPRT |
| | | FEKLEVIMKQYANEKNPLAKYHEETGEYLTKYSKKNNGPIVKSLKYIGNKLGSHLDVTHQFKSS |
| | | TKKLVKLSIKPYRFDVYLTDKGYKFITISYLDVLKKDNYYYIPEQKYDKLKLGKAIDKNAKFIASFY |
| | | KNDLIKLDGEIYKIIGVNSDTRNMIELDLPDIRYKEYCELNNIKGEPRIKKTIGKKVNSIEKLTTDV |
| | | LGNVFTNTQYTKPQLLFKRGN |
| 3699 | Met(−) | NQKFILGLDIGITSVGYGLIDYETKNIIDAGVRLFPEANVENNEGRRSKRGSRRLKRRRIHRLER |
| | SluCas9 | VKKLLEDYNLLDQSQIPQSTNPYAIRVKGLSEALSKDELVIALLHIAKRRGIHKIDVIDSNDDVG |
| | nickase | NELSTKEQLNKNSKLLKDKFVCQIQLERMNEGQVRGEKNRFKTADIIKEIIQLLNVQKNFHQL |
| | | DENFINKYIELVEMRREYFEGPGKGSPYGWEGDPKAWYETLMGHCTYFPDELRSVKYAYSA |
| | | DLFNALNDLNNLVIQRDGLSKLEYHEKYHIIENVFKQKKKPTLKQIANEINVNPEDIKGYRITKS |
| | | GKPQFTEFKLYHDLKSVLFDQSILENEDVLDQIAEILTIYQDKDSIKSKLTELDILLNEEDKENIAQ |
| | | LTGYTGTHRLSLKCIRLVLEEQWYSSRNQMEIFTHLNIKPKKINLTAANKIPKAMIDEFILSPVV |
| | | KRTFGQAINLINKIIEKYGVPEDIIIELARENNSKDKQKFINEMQKKNENTRKRINEIIGKYGNQ |
| | | NAKRLVEKIRLHDEQEGKCLYSLESIPLEDLLNNPNHYEVDHIIPRSVSFDNSYHNKVLVKQSE |
| | | ASKKSNLTPYQYFNSGKSKLSYNQFKQHILNLSKSQDRISKKKKEYLLEERDINKFEVQKEFINR |
| | | NLVDTRYATRELTNYLKAYFSANNMNVKVKTINGSFTDYLRKVWKFKKERNHGYKHHAEDA |
| | | LIIANADFLFKENKKLKAVNSVLEKPEIESKQLDIQVDSEDNYSEMFIIPKQVQDIKDFRNFKYS |
| | | HRVDKKPNRQLINDTLYSTRKKDNSTYIVQTIKDIYAKDNTTLKKQFDKSPEKFLMYQHDPRT |
| | | FEKLEVIMKQYANEKNPLAKYHEETGEYLTKYSKKNNGPIVKSLKYIGNKLGSHLDVTHQFKSS |
| | | TKKLVKLSIKPYRFDVYLTDKGYKFITISYLDVLKKDNYYYIPEQKYDKLKLGKAIDKNAKFIASFY |
| | | KNDLIKLDGEIYKIIGVNSDTRNMIELDLPDIRYKEYCELNNIKGEPRIKKTIGKKVNSIEKLTTDV |
| | | LGNVFTNTQYTKPQLLFKRGN |
| 3700 | Staphylococcus | MKRNYILGLDIGITSVGYGIIDYETRDVIDAGVRLFKEANVENNEGRRSKRGARRLKRRRRHRI |
| | aureus | QRVKKLLFDYNLLTDHSELSGINPYEARVKGLSQKLSEEEFSAALLHLAKRRGVHNVNEVEEDT |
| | Cas9 (SaCas9) | GNELSTKEQISRNSKALEEKYVAELQLERLKKDGEVRGSINRFKTSDYVKEAKQLLKVQKAYHQ |
| | | LDQSFIDTYIDLLETRRTYYEGPGEGSPFGWKDIKEWYEMLMGHCTYFPEELRSVKYAYNADL |
| | | YNALNDLNNLVITRDENEKLEYYEKFQIIENVFKQKKKPTLKQIAKEILVNEEDIKGYRVTSTGK |
| | | PEFTNLKVYHDIKDITARKEIIENAELLDQIAKILTIYQSSEDIQEELTNLNSELTQEEIEQISNLKG |
| | | YTGTHNLSLKAINLILDELWHTNDNQIAIFNRLKLVPKKVDLSQQKEIPTTLVDDFILSPVVKRS |
| | | FIQSIKVINAIIKKYGLPNDIIIELAREKNSKDAQKMINEMQKRNRQTNERIEEIIRTTGKENAKY |
| | | LIEKIKLHDMQEGKCLYSLEAIPLEDLLNNPFNYEVDHIIPRSVSFDNSFNNKVLVKQEENSKKG |
| | | NRTPFQYLSSSDSKISYETFKKHILNLAKGKGRISKTKKEYLLEERDINRFSVQKDFINRNLVDTR |
| | | YATRGLMNLLRSYFRVNNLDVKVKSINGGFTSFLRRKWKFKKERNKGYKHHAEDALIIANAD |
| | | FIFKEWKKLDKAKKVMENQMFEEKQAESMPEIETEQEYKEIFITPHQIKHIKDFKDYKYSHRV |
| | | DKKPNRELINDTLYSTRKDDKGNTLIVNNLNGLYDKDNDKLKKLINKSPEKLLMYHHDPQTYQ |
| | | KLKLIMEQYGDEKNPLYKYYEETGNYLTKYSKKDNGPVIKKIKYYGNKLNAHLDITDDYPNSRN |
| | | KVVKLSLKPYRFDVYLDNGVYKFVTVKNLDVIKKENYYEVNSKCYEEAKKLKKISNQAEFIASFY |
| | | NNDLIKINGELYRVIGVNNDLLNRIEVNMIDITYREYLENMNDKRPPRIIKTIASKTQSIKKYSTD |
| | | ILGNLYEVKSKKHPQIIKKG |
| 3701 | SaCas9 | MKRNYILGLDIGITSVGYGIIDYETRDVIDAGVRLFKEANVENNEGRRSKRGARRLKRRRRHRI |
| | N580A | QRVKKLLFDYNLLTDHSELSGINPYEARVKGLSQKLSEEEFSAALLHLAKRRGVHNVNEVEEDT |
| | nickase | GNELSTKEQISRNSKALEEKYVAELQLERLKKDGEVRGSINRFKTSDYVKEAKQLLKVQKAYHQ |
| | | LDQSFIDTYIDLLETRRTYYEGPGEGSPFGWKDIKEWYEMLMGHCTYFPEELRSVKYAYNADL |
| | | YNALNDLNNLVITRDENEKLEYYEKFQIIENVFKQKKKPTLKQIAKEILVNEEDIKGYRVTSTGK |
| | | PEFTNLKVYHDIKDITARKEIIENAELLDQIAKILTIYQSSEDIQEELTNLNSELTQEEIEQISNLKG |
| | | YTGTHNLSLKAINLILDELWHTNDNQIAIFNRLKLVPKKVDLSQQKEIPTTLVDDFILSPVVKRS |
| | | FIQSIKVINAIIKKYGLPNDIIIELAREKNSKDAQKMINEMQKRNRQTNERIEEIIRTTGKENAKY |
| | | LIEKIKLHDMQEGKCLYSLEAIPLEDLLNNPFNYEVDHIIPRSVSFDNSFNNKVLVKQEEASKKG |
| | | NRTPFQYLSSSDSKISYETFKKHILNLAKGKGRISKTKKEYLLEERDINRFSVQKDFINRNLVDTR |
| | | YATRGLMNLLRSYFRVNNLDVKVKSINGGFTSFLRRKWKFKKERNKGYKHHAEDALIIANAD |
| | | FIFKEWKKLDKAKKVMENQMFEEKQAESMPEIETEQEYKEIFITPHQIKHIKDFKDYKYSHRV |
| | | DKKPNRELINDTLYSTRKDDKGNTLIVNNLNGLYDKDNDKLKKLINKSPEKLLMYHHDPQTYQ |
| | | KLKLIMEQYGDEKNPLYKYYEETGNYLTKYSKKDNGPVIKKIKYYGNKLNAHLDITDDYPNSRN |
| | | KVVKLSLKPYRFDVYLDNGVYKFVTVKNLDVIKKENYYEVNSKCYEEAKKLKKISNQAEFIASFY |
| | | NNDLIKINGELYRVIGVNNDLLNRIEVNMIDITYREYLENMNDKRPPRIIKTIASKTQSIKKYSTD |
| | | ILGNLYEVKSKKHPQIIKKG |
| 3702 | Met(−) | KRNYILGLDIGITSVGYGIIDYETRDVIDAGVRLFKEANVENNEGRRSKRGARRLKRRRRHRIQ |
| | SaCas9 | RVKKLLFDYNLLTDHSELSGINPYEARVKGLSQKLSEEEFSAALLHLAKRRGVHNVNEVEEDTG |
| | nickase | NELSTKEQISRNSKALEEKYVAELQLERLKKDGEVRGSINRFKTSDYVKEAKQLLKVQKAYHQL |
| | | DQSFIDTYIDLLETRRTYYEGPGEGSPFGWKDIKEWYEMLMGHCTYFPEELRSVKYAYNADLY |
| | | NALNDLNNLVITRDENEKLEYYEKFQIIENVFKQKKKPTLKQIAKEILVNEEDIKGYRVTSTGKP |
| | | EFTNLKVYHDIKDITARKEIIENAELLDQIAKILTIYQSSEDIQEELTNLNSELTQEEIEQISNLKGY |
| | | TGTHNLSLKAINLILDELWHTNDNQIAIFNRLKLVPKKVDLSQQKEIPTTLVDDFILSPVVKRSFI |
| | | QSIKVINAIIKKYGLPNDIIIELAREKNSKDAQKMINEMQKRNRQTNERIEEIIRTTGKENAKYLI |
| | | EKIKLHDMQEGKCLYSLEAIPLEDLLNNPFNYEVDHIIPRSVSFDNSFNNKVLVKQEEASKKGN |
| | | RTPFQYLSSSDSKISYETFKKHILNLAKGKGRISKTKKEYLLEERDINRFSVQKDFINRNLVDTRY |
| | | ATRGLMNLLRSYFRVNNLDVKVKSINGGFTSFLRRKWKFKKERNKGYKHHAEDALIIANADFI |
| | | FKEWKKLDKAKKVMENQMFEEKQAESMPEIETEQEYKEIFITPHQIKHIKDFKDYKYSHRVDK |
| | | KPNRELINDTLYSTRKDDKGNTLIVNNLNGLYDKDNDKLKKLINKSPEKLLMYHHDPQTYQKL |
| | | KLIMEQYGDEKNPLYKYYEETGNYLTKYSKKDNGPVIKKIKYYGNKLNAHLDITDDYPNSRNK |
| | | VVKLSLKPYRFDVYLDNGVYKFVTVKNLDVIKKENYYEVNSKCYEEAKKLKKISNQAEFIASFYN |
| | | NDLIKINGELYRVIGVNNDLLNRIEVNMIDITYREYLENMNDKRPPRIIKTIASKTQSIKKYSTDIL |
| | | GNLYEVKSKKHPQIIKKG |
| 3703 | SpCas9-NG | MDKKYSIGLDIGTNSVGWAVITDEYKVPSKKFKVLGNTDRHSIKKNLIGALLFDSGETAEATRL |
| | (VRVRFRR) | KRTARRRYTRRKNRICYLQEIFSNEMAKVDDSFFHRLEESFLVEEDKKHERHPIFGNIVDEVAY |
| | | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESIRPKRNSDKLIARKKDWDPKKYGGFVSPTVAYSVLVV |
| | | AKVEKGKSKKLKSVKELLGITIMERSSFEKNPIDFLEAKGYKEVKKDLIIKLPKYSLFELENGRKR |
| | | MLASARFLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPRAFKYFDTTIDRKVYRSTKE |
| | | VLDATLIHQSITGLYETRIDLSQLGGD |
| 3704 | spCas9-NG | MDKKYSIGLDIGTNSVGWAVITDEYKVPSKKFKVLGNTDRHSIKKNLIGALLFDSGETAEATRL |
| | (H840A_VRVRFRR) | KRTARRRYTRRKNRICYLQEIFSNEMAKVDDSFFHRLEESFLVEEDKKHERHPIFGNIVDEVAY |
| | Nickase | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESIRPKRNSDKLIARKKDWDPKKYGGFVSPTVAYSVLVV |
| | | AKVEKGKSKKLKSVKELLGITIMERSSFEKNPIDFLEAKGYKEVKKDLIIKLPKYSLFELENGRKR |
| | | MLASARFLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPRAFKYFDTTIDRKVYRSTKE |
| | | VLDATLIHQSITGLYETRIDLSQLGGD |
| 3705 | Met(−) | DKKYSIGLDIGTNSVGWAVITDEYKVPSKKFKVLGNTDRHSIKKNLIGALLFDSGETAEATRLK |
| | SpCas9-NG | RTARRRYTRRKNRICYLQEIFSNEMAKVDDSFFHRLEESFLVEEDKKHERHPIFGNIVDEVAYH |
| | Nickase | EKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESIRPKRNSDKLIARKKDWDPKKYGGFVSPTVAYSVLVV |
| | | AKVEKGKSKKLKSVKELLGITIMERSSFEKNPIDFLEAKGYKEVKKDLIIKLPKYSLFELENGRKR |
| | | MLASARFLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPRAFKYFDTTIDRKVYRSTKE |
| | | VLDATLIHQSITGLYETRIDLSQLGGD |
| 3706 | spCas9-NGA | MDKKYSIGLDIGTNSVGWAVITDEYKVPSKKFKVLGNTDRHSIKKNLIGALLFDSGETAEATRL |
| | (VRQR) | KRTARRRYTRRKNRICYLQEIFSNEMAKVDDSFFHRLEESFLVEEDKKHERHPIFGNIVDEVAY |
| | | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFVSPTVAYSVLVVA |
| | | KVEKGKSKKLKSVKELLGITIMERSSFEKNPIDFLEAKGYKEVKKDLIIKLPKYSLFELENGRKRM |
| | | LASARELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKQYRSTKEVL |
| | | DATLIHQSITGLYETRIDLSQLGGD |
| 3707 | spCas9-NGA | MDKKYSIGLDIGTNSVGWAVITDEYKVPSKKFKVLGNTDRHSIKKNLIGALLFDSGETAEATRL |
| | (H840A_VRQR) | KRTARRRYTRRKNRICYLQEIFSNEMAKVDDSFFHRLEESFLVEEDKKHERHPIFGNIVDEVAY |
| | Nickase | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFVSPTVAYSVLVVA |
| | | KVEKGKSKKLKSVKELLGITIMERSSFEKNPIDFLEAKGYKEVKKDLIIKLPKYSLFELENGRKRM |
| | | LASARELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKQYRSTKEVL |
| | | DATLIHQSITGLYETRIDLSQLGGD |
| 3708 | Met(−) | DKKYSIGLDIGTNSVGWAVITDEYKVPSKKFKVLGNTDRHSIKKNLIGALLFDSGETAEATRLK |
| | spCas9-NGA | RTARRRYTRRKNRICYLQEIFSNEMAKVDDSFFHRLEESFLVEEDKKHERHPIFGNIVDEVAYH |
| | Nickase | EKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFVSPTVAYSVLVVA |
| | | KVEKGKSKKLKSVKELLGITIMERSSFEKNPIDFLEAKGYKEVKKDLIIKLPKYSLFELENGRKRM |
| | | LASARELQKGNELALPSKYVNFLYLASHYEKLKGSPEDNEQKQLFVEQHKHYLDEIIEQISEFSK |
| | | RVILADANLDKVLSAYNKHRDKPIREQAENIIHLFTLTNLGAPAAFKYFDTTIDRKQYRSTKEVL |
| | | DATLIHQSITGLYETRIDLSQLGGD |
| 3709 | SpRY Cas9 | MDKKYSIGLDIGTNSVGWAVITDEYKVPSKKFKVLGNTDRHSIKKNLIGALLFDSGETAERTRL |
| | | KRTARRRYTRRKNRICYLQEIFSNEMAKVDDSFFHRLEESFLVEEDKKHERHPIFGNIVDEVAY |
| | | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESIRPKRNSDKLIARKKDWDPKKYGGFLWPTVAYSVLVV |
| | | AKVEKGKSKKLKSVKELLGITIMERSSFEKNPIDFLEAKGYKEVKKDLIIKLPKYSLFELENGRKR |
| | | MLASAKQLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTRLGAPRAFKYFDTTIDPKQYRSTKE |
| | | VLDATLIHQSITGLYETRIDLSQLGGD |
| 3710 | SpRY Cas9 | MDKKYSIGLDIGTNSVGWAVITDEYKVPSKKFKVLGNTDRHSIKKNLIGALLFDSGETAERTRL |
| | (H840A) | KRTARRRYTRRKNRICYLQEIFSNEMAKVDDSFFHRLEESFLVEEDKKHERHPIFGNIVDEVAY |
| | Nickase | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESIRPKRNSDKLIARKKDWDPKKYGGFLWPTVAYSVLVV |
| | | AKVEKGKSKKLKSVKELLGITIMERSSFEKNPIDFLEAKGYKEVKKDLIIKLPKYSLFELENGRKR |
| | | MLASAKQLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTRLGAPRAFKYFDTTIDPKQYRSTKE |
| | | VLDATLIHQSITGLYETRIDLSQLGGD |
| 3711 | Met(−) | DKKYSIGLDIGTNSVGWAVITDEYKVPSKKFKVLGNTDRHSIKKNLIGALLFDSGETAERTRLKR |
| | SpRY | TARRRYTRRKNRICYLQEIFSNEMAKVDDSFFHRLEESFLVEEDKKHERHPIFGNIVDEVAYHE |
| | Cas9 Nickase | KYPTIYHLRKKLVDSTDKADLRLIYLALAHMIKFRGHFLIEGDLNPDNSDVDKLFIQLVQTYNQL |
| | | FEENPINASGVDAKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPILEKMDGTEEL |
| | | LVKLNREDLLRKQRTFDNGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIECFDSVEIS |
| | | GVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESIRPKRNSDKLIARKKDWDPKKYGGFLWPTVAYSVLVV |
| | | AKVEKGKSKKLKSVKELLGITIMERSSFEKNPIDFLEAKGYKEVKKDLIIKLPKYSLFELENGRKR |
| | | MLASAKQLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTRLGAPRAFKYFDTTIDPKQYRSTKE |
| | | VLDATLIHQSITGLYETRIDLSQLGGD |
| 3712 | SRGN3.1 | MNQKFILGLDIGITSVGYGLIDYETKNIIDAGVRLFPEANVENNEGRRSKRGSRRLKRRRIHRLE |
| | | RVKLLLTEYDLINKEQIPTSNNPYQIRVKGLSEILSKDELAIALLHLAKRRGIHNVDVAADKEETA |
| | | SDSLSTKDQINKNAKFLESRYVCELQKERLENEGHVRGVENRFLTKDIVREAKKIIDTQMQYYP |
| | | EIDETFKEKYISLVETRREYFEGPGQGSPFGWNGDLKKWYEMLMGHCTYFPQELRSVKYAYS |
| | | ADLFNALNDLNNLIIQRDNSEKLEYHEKYHIIENVFKQKKKPTLKQIAKEIGVNPEDIKGYRITKS |
| | | GTPEFTSFKLFHDLKKVVKDHAILDDIDLLNQIAEILTIYQDKDSIVAELGQLEYLMSEADKQSIS |
| | | ELTGYTGTHSLSLKCMNMIIDELWHSSMNQMEVFTYLNMRPKKYELKGYQRIPTDMIDDAI |
| | | LSPVVKRTFIQSINVINKVIEKYGIPEDIIIELARENNSDDRKKFINNLQKKNEATRKRINEIIGQT |
| | | GNQNAKRIVEKIRLHDQQEGKCLYSLESIPLEDLLNNPNHYEVDHIIPRSVSFDNSYHNKVLVK |
| | | QSENSKKSNLTPYQYFNSGKSKLSYNQFKQHILNLSKSQDRISKKKKEYLLEERDINKFEVQKEF |
| | | INRNLVDTRYATRELTNYLKAYFSANNMNVKVKTINGSFTDYLRKVWKFKKERNHGYKHHAE |
| | | DALIIANADFLFKENKKLKAVNSVLEKPEIETKQLDIQVDSEDNYSEMFIIPKQVQDIKDFRNFK |
| | | YSHRVDKKPNRQLINDTLYSTRKKDNSTYIVQTIKDIYAKDNTTLKKQFDKSPEKFLMYQHDP |
| | | RTFEKLEVIMKQYANEKNPLAKYHEETGEYLTKYSKKNNGPIVKSLKYIGNKLGSHLDVTHQFK |
| | | SSTKKLVKLSIKNYRFDVYLTEKGYKFVTIAYLNVFKKDNYYYIPKDKYQELKEKKKIKDTDQFIA |
| | | SFYKNDLIKLNGDLYKIIGVNSDDRNIIELDYYDIKYKDYCEINNIKGEPRIKKTIGKKTESIEKFTT |
| | | DVLGNLYLHSTEKAPQLIFKRGL |
| 3713 | SRGN3.1 | MNQKFILGLDIGITSVGYGLIDYETKNIIDAGVRLFPEANVENNEGRRSKRGSRRLKRRRIHRLE |
| | (N585A) | RVKLLLTEYDLINKEQIPTSNNPYQIRVKGLSEILSKDELAIALLHLAKRRGIHNVDVAADKEETA |
| | Nickase | SDSLSTKDQINKNAKFLESRYVCELQKERLENEGHVRGVENRFLTKDIVREAKKIIDTQMQYYP |
| | | EIDETFKEKYISLVETRREYFEGPGQGSPFGWNGDLKKWYEMLMGHCTYFPQELRSVKYAYS |
| | | ADLFNALNDLNNLIIQRDNSEKLEYHEKYHIIENVFKQKKKPTLKQIAKEIGVNPEDIKGYRITKS |
| | | GTPEFTSFKLFHDLKKVVKDHAILDDIDLLNQIAEILTIYQDKDSIVAELGQLEYLMSEADKQSIS |
| | | ELTGYTGTHSLSLKCMNMIIDELWHSSMNQMEVFTYLNMRPKKYELKGYQRIPTDMIDDAI |
| | | LSPVVKRTFIQSINVINKVIEKYGIPEDIIIELARENNSDDRKKFINNLQKKNEATRKRINEIIGQT |
| | | GNQNAKRIVEKIRLHDQQEGKCLYSLESIPLEDLLNNPNHYEVDHIIPRSVSFDNSYHNKVLVK |
| | | QSEASKKSNLTPYQYFNSGKSKLSYNQFKQHILNLSKSQDRISKKKKEYLLEERDINKFEVQKEF |
| | | INRNLVDTRYATRELTNYLKAYFSANNMNVKVKTINGSFTDYLRKVWKFKKERNHGYKHHAE |
| | | DALIIANADFLFKENKKLKAVNSVLEKPEIETKQLDIQVDSEDNYSEMFIIPKQVQDIKDFRNFK |
| | | YSHRVDKKPNRQLINDTLYSTRKKDNSTYIVQTIKDIYAKDNTTLKKQFDKSPEKFLMYQHDP |
| | | RTFEKLEVIMKQYANEKNPLAKYHEETGEYLTKYSKKNNGPIVKSLKYIGNKLGSHLDVTHQFK |
| | | SSTKKLVKLSIKNYRFDVYLTEKGYKFVTIAYLNVFKKDNYYYIPKDKYQELKEKKKIKDTDQFIA |
| | | SFYKNDLIKLNGDLYKIIGVNSDDRNIIELDYYDIKYKDYCEINNIKGEPRIKKTIGKKTESIEKFTT |
| | | DVLGNLYLHSTEKAPQLIFKRGL |
| 3714 | Met(−) | NQKFILGLDIGITSVGYGLIDYETKNIIDAGVRLFPEANVENNEGRRSKRGSRRLKRRRIHRLER |
| | sRGN3.1 | VKLLLTEYDLINKEQIPTSNNPYQIRVKGLSEILSKDELAIALLHLAKRRGIHNVDVAADKEETAS |
| | (N584A) | DSLSTKDQINKNAKFLESRYVCELQKERLENEGHVRGVENRFLTKDIVREAKKIIDTQMQYYPE |
| | Nickase | IDETFKEKYISLVETRREYFEGPGQGSPFGWNGDLKKWYEMLMGHCTYFPQELRSVKYAYSA |
| | | DLFNALNDLNNLIIQRDNSEKLEYHEKYHIIENVFKQKKKPTLKQIAKEIGVNPEDIKGYRITKSG |
| | | TPEFTSFKLFHDLKKVVKDHAILDDIDLLNQIAEILTIYQDKDSIVAELGQLEYLMSEADKQSISE |
| | | LTGYTGTHSLSLKCMNMIIDELWHSSMNQMEVFTYLNMRPKKYELKGYQRIPTDMIDDAIL |
| | | SPVVKRTFIQSINVINKVIEKYGIPEDIIIELARENNSDDRKKFINNLQKKNEATRKRINEIIGQTG |
| | | NQNAKRIVEKIRLHDQQEGKCLYSLESIPLEDLLNNPNHYEVDHIIPRSVSFDNSYHNKVLVKQ |
| | | SEASKKSNLTPYQYFNSGKSKLSYNQFKQHILNLSKSQDRISKKKKEYLLEERDINKFEVQKEFI |
| | | NRNLVDTRYATRELTNYLKAYFSANNMNVKVKTINGSFTDYLRKVWKFKKERNHGYKHHAE |
| | | DALIIANADFLFKENKKLKAVNSVLEKPEIETKQLDIQVDSEDNYSEMFIIPKQVQDIKDERNFK |
| | | YSHRVDKKPNRQLINDTLYSTRKKDNSTYIVQTIKDIYAKDNTTLKKQFDKSPEKFLMYQHDP |
| | | RTFEKLEVIMKQYANEKNPLAKYHEETGEYLTKYSKKNNGPIVKSLKYIGNKLGSHLDVTHQFK |
| | | SSTKKLVKLSIKNYRFDVYLTEKGYKFVTIAYLNVFKKDNYYYIPKDKYQELKEKKKIKDTDQFIA |
| | | SFYKNDLIKLNGDLYKIIGVNSDDRNIIELDYYDIKYKDYCEINNIKGEPRIKKTIGKKTESIEKFTT |
| | | DVLGNLYLHSTEKAPQLIFKRGL |
| 3715 | sRGN3.3 | MNQKFILGLDIGITSVGYGLIDYETKNIIDAGVRLFPEANVENNEGRRSKRGSRRLKRRRIHRLE |
| | | RVKLLLTEYDLINKEQIPTSNNPYQIRVKGLSEILSKDELAIALLHLAKRRGIHNVDVAADKEETA |
| | | SDSLSTKDQINKNAKFLESRYVCELQKERLENEGHVRGVENRFLTKDIVREAKKIIDTQMQYYP |
| | | EIDETFKEKYISLVETRREYFEGPGQGSPFGWNGDLKKWYEMLMGHCTYFPQELRSVKYAYS |
| | | ADLFNALNDLNNLIIQRDNSEKLEYHEKYHIIENVFKQKKKPTLKQIAKEIGVNPEDIKGYRITKS |
| | | GTPEFTSFKLFHDLKKVVKDHAILDDIDLLNQIAEILTIYQDKDSIVAELGQLEYLMSEADKQSIS |
| | | ELTGYTGTHSLSLKCMNMIIDELWHSSMNQMEVFTYLNMRPKKYELKGYQRIPTDMIDDAI |
| | | LSPVVKRTFIQSINVINKVIEKYGIPEDIIIELARENNSDDRKKFINNLQKKNEATRKRINEIIGQT |
| | | GNQNAKRIVEKIRLHDQQEGKCLYSLESIPLEDLLNNPNHYEVDHIIPRSVSFDNSYHNKVLVK |
| | | QSENSKKSNLTPYQYFNSGKSKLSYNQFKQHILNLSKSQDRISKKKKEYLLEERDINKFEVQKEF |
| | | INRNLVDTRYATRELTSYLKAYFSANNMDVKVKTINGSFTNHLRKVWRFDKYRNHGYKHHA |
| | | EDALIIANADFLFKENKKLQNTNKILEKPTIENNTKKVTVEKEEDYNNVFETPKLVEDIKQYRDY |
| | | KFSHRVDKKPNRQLINDTLYSTRMKDEHDYIVQTITDIYGKDNTNLKKQFNKNPEKFLMYQN |
| | | DPKTFEKLSIIMKQYSDEKNPLAKYYEETGEYLTKYSKKNNGPIVKKIKLLGNKVGNHLDVTNK |
| | | YENSTKKLVKLSIKNYRFDVYLTEKGYKFVTIAYLNVFKKDNYYYIPKDKYQELKEKKKIKDTDQF |
| | | IASFYKNDLIKLNGDLYKIIGVNSDDRNIIELDYYDIKYKDYCEINNIKGEPRIKKTIGKKTESIEKFT |
| | | TDVLGNLYLHSTEKAPQLIFKRGL |
| 3716 | SRGN3.3 | MNQKFILGLDIGITSVGYGLIDYETKNIIDAGVRLFPEANVENNEGRRSKRGSRRLKRRRIHRLE |
| | (N585A) | RVKLLLTEYDLINKEQIPTSNNPYQIRVKGLSEILSKDELAIALLHLAKRRGIHNVDVAADKEETA |
| | Nickase | SDSLSTKDQINKNAKFLESRYVCELQKERLENEGHVRGVENRFLTKDIVREAKKIIDTQMQYYP |
| | | EIDETFKEKYISLVETRREYFEGPGQGSPFGWNGDLKKWYEMLMGHCTYFPQELRSVKYAYS |
| | | ADLFNALNDLNNLIIQRDNSEKLEYHEKYHIIENVFKQKKKPTLKQIAKEIGVNPEDIKGYRITKS |
| | | GTPEFTSFKLFHDLKKVVKDHAILDDIDLLNQIAEILTIYQDKDSIVAELGQLEYLMSEADKQSIS |
| | | ELTGYTGTHSLSLKCMNMIIDELWHSSMNQMEVFTYLNMRPKKYELKGYQRIPTDMIDDAI |
| | | LSPVVKRTFIQSINVINKVIEKYGIPEDIIIELARENNSDDRKKFINNLQKKNEATRKRINEIIGQT |
| | | GNQNAKRIVEKIRLHDQQEGKCLYSLESIPLEDLLNNPNHYEVDHIIPRSVSFDNSYHNKVLVK |
| | | QSEASKKSNLTPYQYFNSGKSKLSYNQFKQHILNLSKSQDRISKKKKEYLLEERDINKFEVQKEF |
| | | INRNLVDTRYATRELTSYLKAYFSANNMDVKVKTINGSFTNHLRKVWRFDKYRNHGYKHHA |
| | | EDALIIANADFLFKENKKLQNTNKILEKPTIENNTKKVTVEKEEDYNNVFETPKLVEDIKQYRDY |
| | | KFSHRVDKKPNRQLINDTLYSTRMKDEHDYIVQTITDIYGKDNTNLKKQFNKNPEKFLMYQN |
| | | DPKTFEKLSIIMKQYSDEKNPLAKYYEETGEYLTKYSKKNNGPIVKKIKLLGNKVGNHLDVTNK |
| | | YENSTKKLVKLSIKNYRFDVYLTEKGYKFVTIAYLNVFKKDNYYYIPKDKYQELKEKKKIKDTDQF |
| | | IASFYKNDLIKLNGDLYKIIGVNSDDRNIIELDYYDIKYKDYCEINNIKGEPRIKKTIGKKTESIEKFT |
| | | TDVLGNLYLHSTEKAPQLIFKRGL |
| 3717 | Met(−) | NQKFILGLDIGITSVGYGLIDYETKNIIDAGVRLFPEANVENNEGRRSKRGSRRLKRRRIHRLER |
| | SRGN3.3 | VKLLLTEYDLINKEQIPTSNNPYQIRVKGLSEILSKDELAIALLHLAKRRGIHNVDVAADKEETAS |
| | (N584A) | DSLSTKDQINKNAKFLESRYVCELQKERLENEGHVRGVENRFLTKDIVREAKKIIDTQMQYYPE |
| | Nickase | IDETFKEKYISLVETRREYFEGPGQGSPFGWNGDLKKWYEMLMGHCTYFPQELRSVKYAYSA |
| | | DLFNALNDLNNLIIQRDNSEKLEYHEKYHIIENVFKQKKKPTLKQIAKEIGVNPEDIKGYRITKSG |
| | | TPEFTSFKLFHDLKKVVKDHAILDDIDLLNQIAEILTIYQDKDSIVAELGQLEYLMSEADKQSISE |
| | | LTGYTGTHSLSLKCMNMIIDELWHSSMNQMEVFTYLNMRPKKYELKGYQRIPTDMIDDAIL |
| | | SPVVKRTFIQSINVINKVIEKYGIPEDIIIELARENNSDDRKKFINNLQKKNEATRKRINEIIGQTG |
| | | NQNAKRIVEKIRLHDQQEGKCLYSLESIPLEDLLNNPNHYEVDHIIPRSVSFDNSYHNKVLVKQ |
| | | SEASKKSNLTPYQYFNSGKSKLSYNQFKQHILNLSKSQDRISKKKKEYLLEERDINKFEVQKEFI |
| | | NRNLVDTRYATRELTSYLKAYFSANNMDVKVKTINGSFTNHLRKVWRFDKYRNHGYKHHAE |
| | | DALIIANADFLFKENKKLQNTNKILEKPTIENNTKKVTVEKEEDYNNVFETPKLVEDIKQYRDYK |
| | | FSHRVDKKPNRQLINDTLYSTRMKDEHDYIVQTITDIYGKDNTNLKKQFNKNPEKFLMYQND |
| | | PKTFEKLSIIMKQYSDEKNPLAKYYEETGEYLTKYSKKNNGPIVKKIKLLGNKVGNHLDVTNKYE |
| | | NSTKKLVKLSIKNYRFDVYLTEKGYKFVTIAYLNVFKKDNYYYIPKDKYQELKEKKKIKDTDQFIA |
| | | SFYKNDLIKLNGDLYKIIGVNSDDRNIIELDYYDIKYKDYCEINNIKGEPRIKKTIGKKTESIEKFTT |
| | | DVLGNLYLHSTEKAPQLIFKRGL |
| 3718 | SpG | MDKKYSIGLDIGTNSVGWAVITDEYKVPSKKFKVLGNTDRHSIKKNLIGALLFDSGETAEATRL |
| | | KRTARRRYTRRKNRICYLQEIFSNEMAKVDDSFFHRLEESFLVEEDKKHERHPIFGNIVDEVAY |
| | | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFLWPTVAYSVLVV |
| | | AKVEKGKSKKLKSVKELLGITIMERSSFEKNPIDFLEAKGYKEVKKDLIIKLPKYSLFELENGRKR |
| | | MLASAKQLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPAAFKYFDTTIDRKQYRSTKE |
| | | VLDATLIHQSITGLYETRIDLSQLGGD |
| 3719 | SpG(H840A) | MDKKYSIGLDIGTNSVGWAVITDEYKVPSKKFKVLGNTDRHSIKKNLIGALLFDSGETAEATRL |
| | Nickase | KRTARRRYTRRKNRICYLQEIFSNEMAKVDDSFFHRLEESFLVEEDKKHERHPIFGNIVDEVAY |
| | | HEKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFLWPTVAYSVLVV |
| | | AKVEKGKSKKLKSVKELLGITIMERSSFEKNPIDFLEAKGYKEVKKDLIIKLPKYSLFELENGRKR |
| | | MLASAKQLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPAAFKYFDTTIDRKQYRSTKE |
| | | VLDATLIHQSITGLYETRIDLSQLGGD |
| 3720 | Met(−) | DKKYSIGLDIGTNSVGWAVITDEYKVPSKKFKVLGNTDRHSIKKNLIGALLFDSGETAEATRLK |
| | SpG(H839A) | RTARRRYTRRKNRICYLQEIFSNEMAKVDDSFFHRLEESFLVEEDKKHERHPIFGNIVDEVAYH |
| | Nickase | EKYPTIYHLRKKLVDSTDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIALSLGLTPNFKSNFDL |
| | | AEDAKLQLSKDTYDDDLDNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQEEFYKFIKPILEKMDGTE |
| | | ELLVKLNREDLLRKQRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNLPNEKVLPKHSLLY |
| | | EYFTVYNELTKVKYVTEGMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDREMIEERLKTYAHLFDDK |
| | | VMKQLKRRRYTGWGRLSRKLINGIRDKQSGKTILDFLKSDGFANRNFMQLIHDDSLTFKEDI |
| | | QKAQVSGQGDSLHEHIANLAGSPAIKKGILQTVKVVDELVKVMGRHKPENIVIEMARENQT |
| | | TQKGQKNSRERMKRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREVKVIT |
| | | LKSKLVSDFRKDFQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGEIVWDKGRDFATVR |
| | | KVLSMPQVNIVKKTEVQTGGFSKESILPKRNSDKLIARKKDWDPKKYGGFLWPTVAYSVLVV |
| | | AKVEKGKSKKLKSVKELLGITIMERSSFEKNPIDFLEAKGYKEVKKDLIIKLPKYSLFELENGRKR |
| | | MLASAKQLQKGNELALPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPAAFKYFDTTIDRKQYRSTKE |
| | | VLDATLIHQSITGLYETRIDLSQLGGD |

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e. the PAM strand), but not the non-edit strand of a double stranded target DNA sequence. In some embodiments, a prime editor comprises a Cas9 having a non-functional RuvC domain that can cleave the target strand (i.e. the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprises a mutation at amino acid D10 as compared to a wild type SpCas9 as set forth in SEQ ID NO: 3693, or a corresponding mutation thereof. In some embodiments, the Cas9 comprises a D10A mutation as compared to a wild type SpCas9 as set forth in SEQ ID NO: 3693, or a corresponding mutation thereof. In some embodiments, the Cas9 polypeptide comprises a mutation at amino acid D10, G12, and/or G17 as compared to a wild type SpCas9 as set forth in SEQ ID NO: 3693, or a corresponding mutation thereof. In some embodiments, the Cas9 polypeptide comprises a D10A mutation, a G12A mutation, and/or a G17A mutation as compared to a wild type SpCas9 as set forth in SEQ ID NO: 3693, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprises a mutation at amino acid H840 as compared to a wild type SpCas9 as set forth in SEQ ID NO: 3693, or a corresponding mutation thereof. In some embodiments, the Cas9 polypeptide comprises a H840A mutation as compared to a wild type SpCas9 as set forth in SEQ ID NO: 3693, or a corresponding mutation thereof. In some embodiments, the Cas9 polypeptide comprises a mutation at amino acid E762, D839, H840, N854, N856, N863, H982, H983, A984, D986, and/or a A987 as compared to a wild type SpCas9 as set forth in SEQ ID NO: 3693, or a corresponding mutation thereof. In some embodiments, the Cas9 polypeptide comprises a E762A, D839A, H840A, N854A, N856A, N863A, H982A, H983A, A984A, and/or a D986A mutation as compared to a wild type SpCas9 as set forth in SEQ ID NO: 3693, or a corresponding mutation thereof. In some embodiments, the Cas9 polypeptide comprises a mutation at amino acid residue R221, N394, and/or H840 as compared to a wild type SpCas9 (e.g., SEQ ID NO: 3693). In some embodiments, the Cas9 polypeptide comprises a R221K, N394L, and/or H840A mutation as compared to a wild type SpCas9 as set forth in SEQ ID NO: 3693, or a corresponding mutation thereof. In some embodiments, the Cas9 polypeptide comprises a mutation at amino acid residue R220, N393, and/or H839 as compared to a wild type SpCas9 (e.g., SEQ ID NO: 3693) lacking a N-terminal methionine, or a corresponding mutation thereof. In some embodiments, the Cas9 polypeptide comprises a R220K, N393K, and/or H839A mutation as compared to a wild type SpCas9 (as set forth in SEQ ID NO: 3693) lacking a N-terminal methionine, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 3693 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 3693, or corresponding mutations thereof.

In some embodiments, the N-terminal methionine is removed from the amino acid sequence of a Cas9 nickase, or from any Cas9 variant, ortholog, or equivalent disclosed or contemplated herein. For example, methionine-minus (Met (−)) Cas9 nickases include any one of the sequences set forth in SEQ ID NOs: 3695, 3696, 3699, 3702, 3705, 3708, 3711, 3714, 3717, 3720, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70%, at least about 80%, at least about 90%, at least about 95%, at least about 96%, at least about 97%, at least about 98%, at least about 99%, at least about 99.5%, or at least about 99.9% sequence identity to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 54 below. It should be appreciated that for each of the variants provided, the Cas protein comprises one or more of the amino acid substitutions as indicated compared to a wild type Cas protein sequence, for example, the wild type Cas9 as set forth in SEQ ID NO: 3693. The PAM motifs as shown in Table 54 below are in the order of 5′ to 3′. In some embodiments, the Cas proteins of the disclosure can also be used to direct transcriptional control of target sequences, for example silencing transcription by sequence-specific binding to target sequences. In some embodiments, a Cas protein described herein may have one or mutations in a PAM recognition motif. In some embodiments, a Cas protein described herein may have altered PAM specificity.

As used in PAM sequences in Table 54, “N” refers to any one of the nucleotides A, G, C, and T; “R” refers to nucleotide A or G; “W” refers to A or T; “V” refers to A, C, or G and “Y” refers to nucleotide C or T

TABLE 54
Cas protein variants and corresponding PAM sequences

| Variant | PAM |
|---|---|
| spCas9 (wild type) | NGG, NGA, |
| | NAG, NGNGA |
| spCas9-VRVRFRR R1335V, L1111R, D1135V, G1218R, | NG |
| E1219F, A1322R, T1337R | |
| spCas9-VQR (D1135V, R1335Q, T1337R) | NGA |
| spCas9-EQR (D1135E, R1335Q, T1337R) | NGA |
| spCas9-VRER (D1135V, G1218R, R1335E, T1337R) | NGCG |
| spCas9-VRQR (D1135V, G1218R, R1335Q, T1337R) | NGA |
| Cas9-NG (L1111R, D1135V, G1218R, E1219F, A1322R, | NGN |
| T1337R, R1335V) | |
| SpG Cas9 (D1135L, S1136W, G1218K, E1219Q, | NGN |
| R1335Q, T1337R) | |
| SyRY Cas9 | NRN |
| (A61R, L1111R, N1317R, A1322R, and R1333P) | |
| xCas9 (E480K, E543D, E1219V, K294R, Q1256K, | NGN |
| A262T, S409I, M694I) | |
| SluCa9 | NNGG |
| sRGN1, sRGN2, sRGN4, sRGN3.1, sRGN3.3 | NNGG |
| saCas9 | NNGRRT, |
| | NNGRRN |
| saCas9-KKH (E782K, N968K, R1015H) | NNNRRT |
| spCas9-MQKSER (D1135M, S1136Q, G1218K, E1219S, | NGCG/NGCN |
| R1335E, T1337R) | |
| spCas9-LRKIQK (D1135L, S1136R, G1218K, E1219I, | NGTN |
| R1335Q, T1337K) | |
| spCas9-LRVSQK (D1135L, S1136R, G1218V, E1219S, | NGTN |
| R1335Q, T1337K) | |
| spCas9-LRVSQL(D1135L, S1136R, G1218V, E1219S, | NGTN |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A61R, L111R, D1135V, R221K, A262T, R324L, N394K, S409I, S409I, E427G, E480K, M495V, N497A, Y515N, K526E, F539S, E543D), R654L, R661A, R661L, R691A, N692A, M694A, M6941, Q695A, H698A, R753G, M7631, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q12211H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 3693.

In some embodiments, a prime editor comprises a SaCas9 polypeptide. In some embodiments, the SaCas9 polypeptide comprises one or more of mutations E782K, N968K, and R1015H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations 1367K, G368D, 1369K, H371L, T375S, T376G, and T1227K as compared to the wild type ScCas9. In some embodiments, a prime editor comprises a St1 Cas9 polypeptide, a St3 Cas9 polypeptide, or a SluCas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration can be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 3693):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 3693-1368 amino acids of UniProtKB-Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 3693-1368 amino acids of UniProtKB-Q99ZW2 N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the 95% or more of the C-terminal amino acids of a Cas9 (e.g., amino acids about 1300-1368 as set forth in SEQ ID No: 3693 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID NO: 3693 or a ortholog or a variant thereof). The N-terminal portion may correspond to 95% or more of the N-terminal amino acids of a Cas9 (e.g., amino acids about 1-1300 as set forth in SEQ ID No: 3693 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N terminal amino acids of a Cas9 (e.g., as set forth in SEQ ID NO: 3693 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID NO: 3693 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%,5%, 4%, 3%, 2%, or 1% of the amino acids of a Cas9 (e.g., as set forth in SEQ ID NO: 3693 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID NO: 3693 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., as set forth in SEQ ID NO: 3693 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID NO: 3693 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 3693: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID NO: 3693 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³ Cas9-CP¹⁰²⁹, Cas9-CP¹⁰⁴¹, Cas9-CP¹²⁴⁷, Cas9-CP¹²⁴⁹ and Cas9-CP¹²⁸² respectively. This description is not meant to be limited to making CP variants from SEQ ID NO: 3693, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 3693). In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

Exemplary Cas proteins and nomenclature are shown in Table 55 below:

TABLE 55
Exemplary Cas proteins and nomenclature

| Legacy nomenclature | Current nomenclature |
|---|---|
| type II CRISPR-Cas enzymes | |
| Cas9 | same |
| type V CRISPR-Cas enzymes | |
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |
| type VI CRISPR-Cas enzymes | |
| C2c2 | Cas13a |
| Cas13d | same |
| C2c7 | Cas13c |
| C2c6 | Cas13b |

In some embodiments, prime editors described herein can also comprise Cas proteins other than Cas9. For example, in some embodiments, a prime editor as described herein can comprise a Cas12 polypeptide such as a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasD polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas Φ protein. In some embodiments, the Cas protein is a Cas12e, Cas12d, Cas13, or Cas Φ nickase.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor can further comprise more than 4, 5, 6, 7, 8, 9 or 10 NLSs.

In addition, the NLSs can be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence

(SEQ ID NO: 3721)
MDSLLMNRRKFLYQFKNVRWAKGRRETYLC,
(SEQ ID NO: 3722)
KRTADGSEFESPKKKRKV,
(SEQ ID NO: 3723)
KRTADGSEFEPKKKRKV,
(SEQ ID NO: 3724)
NLSKRPAAIKKAGQAKKKK,
(SEQ ID NO: 3725)
RQRRNELKRSF,
or
(SEQ ID NO: 3726)
NQSSNFGPMKGGNFGGRSSGPYGGGGQYFAKPRNQGGY.

In some embodiments, a NLS is a monopartite NLS. For example, in some embodiments, a NLS is a SV40 large T antigen NLS PKKKRKV (SEQ ID NO: 3727). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the sequence KRXXXXXXXXXXKKKL (Xenopus nucleoplasmin NLS) (SEQ ID NO: 3728), wherein X is any amino acid. In some embodiments, the NLS comprises a nucleoplasmin NLS sequence KRPAATKKAGQAKKKK (SEQ ID NO: 3729). In some embodiments, a NLS is a noncanonical sequences such as M9 of the hnRNP Al protein, the influenza virus nucleoprotein NLS, and the yeast Gal4 protein NLS. In some embodiments, a NLS is a noncanonical sequences such as M9 of the hnRNP Al protein, the influenza virus nucleoprotein NLS, and the yeast Gal4 protein NLS.

Other non-limiting examples of NLS sequences are provided in Table 56 below. In some embodiments, a bipartite NLS consists of two basic domains separated by a spacer sequence comprising a variable number of amino acids. In some embodiments, a NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 3721-3739. In some embodiments, a NLS comprises an amino acid sequence selected from the group consisting of SEQ ID NOs: 3721-3739. In some embodiments, a prime editing composition comprises a polynucleotide that encodes a NLS that comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 3721-3739. In some embodiments, a prime editing composition comprises a polynucleotide that encodes a NLS that comprises an amino acid sequence selected from the group consisting of 3721-3739.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues. Non-limiting examples of NLS sequences are provided in Table 56 below.

TABLE 56
Exemplary nuclear localization sequences

| Description | Sequence | SEQ ID NO: |
|---|---|---|
| NLS of SV40 Large T-AG | PKKKRKV | 3727 |
| NLS | MKRTADGSEFESPKKKRKV | 3730 |
| NLS | MDSLLMNRRKFLYQFKNVRWAKGRRETYLC | 3721 |
| NLS of Nucleoplasmin | AVKRPAATKKAGQAKKKKLD | 3731 |
| NLS of EGL-13 | MSRRRKANPTKLSENAKKLAKEVEN | 3732 |
| NLS of C-Myc | PAAKRVKLD | 3733 |
| NLS of Tus-protein | KLKIKRPVK | 3734 |
| NLS of polyoma large T-AG | VSRKRPRP | 3735 |
| NLS of Hepatitis D virus | EGAPPAKRAR | 3736 |
| antigen | | |
| NLS of Rev protein | RQARRNRRRRWRERNR | 3737 |
| NLS of murine p53 | PPQPKKKPLDGE | 3738 |
| C terminal linker and NLS | SGGSKRTADGSEFEPKKKRKV | 3739 |
| of an exemplary prime | | |
| editor fusion protein | | |
| NLS | KRTADGSEFEPKKKRKV | 3723 |

In some embodiments, a prime editing complex comprises a fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 3740. In some embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to SEQ ID NO: 3740 set forth in Table 57. In some embodiments, a prime editor fusion protein comprises an amino acid sequence that lacks a N-terminus methionine relative to an amino acid sequence set forth in SEQ ID NO: 3740. Sequence of an exemplary prime editor fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)] and its components are shown in Table 57.

In some embodiments, a prime editing complex comprises a fusion protein comprising a DNA binding domain (e.g., Cas9((R221K N394K H840A)) and a reverse transcriptase (e.g., a variant MMLV RT) having the following structure: [NLS]-[Cas9((R221K N394K H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 3741. In some embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to SEQ ID NO: 3741 set forth in Table 58. In some embodiments, a prime editor fusion protein comprises an amino acid sequence that lacks a N-terminus methionine relative to an amino acid sequence set forth in SEQ ID NO: 3741. Sequence of an exemplary prime editor fusion protein comprising a DNA binding domain (e.g., Cas9(H840A)) and a reverse transcriptase (e.g., a variant MMLV RT) having the following structure: [NLS]-[Cas9 (R221K N394K H840A)]-[linker]-[MMLV_RT(D200N)(T330P)(L603W)(T306K)(W313F)] and its components are shown in Table 58.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer.

In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is:

(SEQ ID NO: 3742)
GCCAACATGAGGATCACCCATGTCTGCAGGGCC.

amino acid sequence of the MCP is:

(SEQ ID NO: 3743)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIAANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 3744), (G)n (SEQ ID NO: 3745), (EAAAK)n (SEQ ID NO: 3746), (GGS)n (SEQ ID NO: 3747), (SGGS)n (SEQ ID NO: 3748), (XP)n (SEQ ID NO: 3749), or any combination thereof, wherein n is independently an integer between 1 and 30, and wherein X is any amino acid. In some embodiments, the linker comprises the amino acid sequence (GGS)n (SEQ ID NO: 3747), wherein n is 1, 3, or 7. In some embodiments, the linker comprises the amino acid sequence SGSETPGTSESATPES (SEQ ID NO: 3750). In some embodiments, the linker comprises the amino acid sequence SGGSSGGSSGSETPGTSESATPESSGGSSGGS (SEQ ID NO: 3751). In some embodiments, the linker comprises the amino acid sequence SGGSGGSGGS (SEQ ID NO: 3752). In some embodiments, the linker comprises the amino acid sequence SGGS (SEQ ID NO: 3753). In other embodiments, the linker comprises the amino acid sequence

(SEQ ID NO: 3754)
SGGSSGGSSGSETPGTSESATPESAGSYPYDVPDYAGSAAPAAKKKKLD
GSGSGGSSGGS.

In some embodiments, a linker comprises 1-100 amino acids. In some embodiments, the linker comprises the amino acid sequence SGSETPGTSESATPES (SEQ ID NO: 3750). In some embodiments, the linker comprises the amino acid sequence SGGSSGGSSGSETPGTSESATPESSGGSSGGS (SEQ ID NO: 3751). In some embodiments, the linker comprises the amino acid sequence SGGSGGSGGS (SEQ ID NO: 3752). In some embodiments, the linker comprises the amino acid sequence SGGS (SEQ ID NO: 3753). In some embodiments, the linker comprises the amino acid sequence GGSGGS (SEQ ID NO: 3755), GGSGGSGGS (SEQ ID NO: 3756); SGGSSGGSSGSETPGTSESATPESAGSYPYDVPDYAGSAAPAAKKKKLDGSGSGGSSGGS (SEQ ID NO: 3754), or SGGSSGGSSGSETPGTSESATPESSGGSSGGSS (SEQ ID NO: 3757). In some embodiments, the linker comprises the sequence SGGSSGGSSGSETPGTSESATPESSGGSSGGSS (SEQ ID NO: 3757).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COGH; or NH2-[polymerase]-[DNA binding domain]-COGH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COGH. In some embodiments, a prime editor comprises a fusion protein and a DNA binding domain provided in trans, wherein the fusion protein comprises the structure NH2-[DNA polymerase domain]-[RNA-protein recruitment polypeptide]-COGH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g. AAV vectors) encoding each thereof. When delivered and/or expressed in a target cell, the intein-N and the intein-C can be excised via protein trans-splicing, resulting in a complete prime editor fusion protein in the target cell.

In some embodiments, an exemplary protein described herein may lack a methionine residue at the N-terminus. In some embodiments, a prime editor fusion protein comprises a Cas9(H840A) nickase and a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type or reference M-MLV RT. In some embodiments, a prime editor fusion protein comprises a Cas9(H840A) nickase and a M-MLV RT that comprises amino acid substitutions D200N, T330P, T306K, W313F, and L603W compared to a wild type or reference M-MLV RT. The amino acid sequence of an exemplary prime editor fusion protein and its individual components is shown in Table 57.

In some embodiments, a prime editor fusion protein comprises a Cas9 (R221K N394K H840A) nickase and a M-MLV RT that comprises amino acid substitutions D200N, T330P, T306K, W313F, and L603W compared to a wild type or reference M-MLV RT. The amino acid sequence of an exemplary Prime editor fusion protein and its individual components is shown in Table 58.

In some embodiments an exemplary prime editor protein may comprise an amino acid sequence as set forth in any of the SEQ ID NO: 3740 or SEQ ID NO: 3741.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the prime editor fusion sequences described herein or known in the art.

TABLE 57
lists exemplary prime editor and its components

| SEQ | | |
|---|---|---|
| ID | | |
| NO: | DESCRIPTION | SEQUENCE |
| 3740 | Exemplary Prime | MKRTADGSEFESPKKKRKVDKKYSIGLDIGTNSVGWAVITDEYKVPSKKFKV |
| | Editor [NLS]- | LGNTDRHSIKKNLIGALLFDSGETAEATRLKRTARRRYTRRKNRICYLQEIFSN |
| | [Cas9(H840A)]- | EMAKVDDSFFHRLEESFLVEEDKKHERHPIFGNIVDEVAYHEKYPTIYHLRKK |
| | [linker]- | LVDSTDKADLRLIYLALAHMIKFRGHFLIEGDLNPDNSDVDKLFIQLVQTYN |
| | [MMLV_RT(D200N)(T | QLFEENPINASGVDAKAILSARLSKSRRLENLIAQLPGEKKNGLFGNLIALSLG |
| | 330P)(L603W)(T306K) | LTPNFKSNFDLAEDAKLQLSKDTYDDDLDNLLAQIGDQYADLFLAAKNLSDA |
| | (W313F)]-[NLS] | ILLSDILRVNTEITKAPLSASMIKRYDEHHQDLTLLKALVRQQLPEKYKEIFFD |
| | | QSKNGYAGYIDGGASQEEFYKFIKPILEKMDGTEELLVKLNREDLLRKQRTFD |
| | | NGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPK |
| | | HSLLYEYFTVYNELTKVKYVTEGMRKPAFLSGEQKKAIVDLLFKTNRKVTVK |
| | | QLKEDYFKKIECFDSVEISGVEDRFNASLGTYHDLLKIIKDKDFLDNEENEDILE |
| | | DIVLTLTLFEDREMIEERLKTYAHLFDDKVMKQLKRRRYTGWGRLSRKLINGI |
| | | RDKQSGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQTTQK |
| | | GQKNSRERMKRIEEGIKELGSQILKEHPVENTQLQNEKLYLYYLQNGRDMY |
| | | VDQELDINRLSDYDVDAIVPQSFLKDDSIDNKVLTRSDKNRGKSDNVPSEEV |
| | | VKKMKNYWRQLLNAKLITQRKFDNLTKAERGGLSELDKAGFIKRQLVETRQ |
| | | ITKHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEIG |
| | | KATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGF |
| | | DSPTVAYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFLYLASHY |
| | | EKLKGSPEDNEQKOLFVEQHKHYLDEIIEQISEFSKRVILADANLDKVLSAYN |
| | | KHRDKPIREQAENIIHLFTLTNLGAPAAFKYFDTTIDRKRYTSTKEVLDATLIH |
| | | QSITGLYETRIDLSQLGGDSGGSSGGSSGSETPGTSESATPESSGGSSGGSSTL |
| | | NIEDEYRLHETSKEPDVSLGSTWLSDFPQAWAETGGMGLAVRQAPLIIPLKAT |
| | | STPVSIKQYPMSQEARLGIKPHIQRLLDQGILVPCQSPWNTPLLPVKKPGTND |
| | | YRPVQDLREVNKRVEDIHPTVPNPYNLLSGLPPSHQWYTVLDLKDAFFCLRLH |
| | | PTSQPLFAFEWRDPEMGISGQLTWTRLPQGFKNSPTLFNEALHRDLADFRIQ |
| | | HPDLILLQYVDDLLLAATSELDCQQGTRALLQTLGNLGYRASAKKAQICQKQV |
| | | KYLGYLLKEGQRWLTEARKETVMGQPTPKTPRQLREFLGKAGFCRLFIPGFAE |
| | | MAAPLYPLTKPGTLFNWGPDQQKAYQEIKQALLTAPALGLPDLTKPFELFVDE |
| | | KQGYAKGVLTQKLGPWRRPVAYLSKKLDPVAAGWPPCLRMVAAIAVLTKDA |
| | | GKLTMGQPLVILAPHAVEALVKQPPDRWLSNARMTHYQALLLDTDRVQFGP |
| | | VVALNPATLLPLPEEGLOHNCLDILAEAHGTRPDLTDQPLPDADHTWYTDGS |
| | | SLLQEGQRKAGAAVTTETEVIWAKALPAGTSAQRAELIALTQALKMAEGKKL |
| | | NVYTDSRYAFATAHIHGEIYRRRGWLTSEGKEIKNKDEILALLKALFLPKRLSIIH |
| | | CPGHQKGHSAEARGNRMADQAARKAAITETPDTSTLLIENSSPSGGSKRTAD |
| | | GSEFEPKKKRKV |
| | | KEY: |
| | | NUCLEAR LOCALIZATION SEQUENCE (NLS) |
| | | CAS9(H840A) |
| | | 33-AMINO ACID LINKER |
| | | M-MLV REVERSE TRANSCRIPTASE |
| 3730 | -N-terminal NLS | MKRTADGSEFESPKKKRKV |
| 3695 | -CAS9 (H840A) (MET | DKKYSIGLDIGTNSVGWAVITDEYKVPSKKFKVLGNTDRHSIKKNLIGALLFDS |
| | MINUS) | GETAEATRLKRTARRRYTRRKNRICYLQEIFSNEMAKVDDSFFHRLEESFLVEE |
| | | DKKHERHPIFGNIVDEVAYHEKYPTIYHLRKKLVDSTDKADLRLIYLALAHMIKF |
| | | RGHFLIEGDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQLSKDTYD |
| | | DDLDNLLAQIGDQYADLFLAAKNLSDAILLSDILRVNTEITKAPLSASMIKRYDE |
| | | HHQDLTLLKALVRQQLPEKYKEIFFDQSKNGYAGYIDGGASQEEFYKFIKPILEK |
| | | MDGTEELLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDKGASAQS |
| | | FIERMTNFDKNLPNEKVLPKHSLLYEYFTVYNELTKVKYVTEGMRKPAFLSGEQ |
| | | KKAIVDLLFKTNRKVTVKQLKEDYFKKIECFDSVEISGVEDRFNASLGTYHDLLKI |
| | | IKDKDFLDNEENEDILEDIVLTLTLFEDREMIEERLKTYAHLFDDKVMKQLKRRR |
| | | YTGWGRLSRKLINGIRDKQSGKTILDFLKSDGFANRNFMQLIHDDSLTFKEDIQ |
| | | KAQVSGQGDSLHEHIANLAGSPAIKKGILQTVKVVDELVKVMGRHKPENIVIE |
| | | MARENQTTQKGQKNSRERMKRIEEGIKELGSQILKEHPVENTQLQNEKLYLYY |
| | | LONGRDMYVDQELDINRLSDYDVDAIVPQSFLKDDSIDNKVLTRSDKNRGKS |
| | | DNVPSEEVVKKMKNYWRQLLNAKLITQRKFDNLTKAERGGLSELDKAGFIKR |
| | | QLVETRQITKHVAQILDSRMNTKYDENDKLIREVKVITLKSKLVSDFRKDFQFY |
| | | KVREINNYHHAHDAYLNAVVGTALIKKYPKLESEFVYGDYKVYDVRKMIAKSE |
| | | QEIGKATAKYFFYSNIMNFFKTEITLANGEIRKRPLIETNGETGEIVWDKGRDF |
| | | ATVRKVLSMPQVNIVKKTEVQTGGFSKESILPKRNSDKLIARKKDWDPKKYGG |
| | | FDSPTVAYSVLVVAKVEKGKSKKLKSVKELLGITIMERSSFEKNPIDFLEAKGYKE |
| | | VKKDLIIKLPKYSLFELENGRKRMLASAGELQKGNELALPSKYVNFLYLASHYEK |
| | | LKGSPEDNEQKOLFVEQHKHYLDEIIEQISEFSKRVILADANLDKVLSAYNKHR |
| | | DKPIREQAENIIHLFTLTNLGAPAAFKYFDTTIDRKRYTSTKEVLDATLIHQSITG |
| | | LYETRIDLSQLGGD |
| 3757 | -linker between | SGGSSGGSSGSETPGTSESATPESSGGSSGGSS |
| | CAS9 domain and RT | |
| | domain (33 amino | |
| | acids) | |
| 3692 | -MMLV_RT D200N | TLNIEDEYRLHETSKEPDVSLGSTWLSDFPQAWAETGGMGLAVRQAPLIIPLK |
| | T330P L603W T306K | ATSTPVSIKQYPMSQEARLGIKPHIQRLLDQGILVPCQSPWNTPLLPVKKPGT |
| | W313F | NDYRPVQDLREVNKRVEDIHPTVPNPYNLLSGLPPSHQWYTVLDLKDAFFCL |
| | | RLHPTSQPLFAFEWRDPEMGISGQLTWTRLPQGFKNSPTLFNEALHRDLADF |
| | | RIQHPDLILLQYVDDLLLAATSELDCQQGTRALLQTLGNLGYRASAKKAQICQK |
| | | QVKYLGYLLKEGQRWLTEARKETVMGQPTPKTPROLREFLGKAGFCRLFIPGF |
| | | AEMAAPLYPLTKPGTLFNWGPDQQKAYQEIKQALLTAPALGLPDLTKPFELFV |
| | | DEKQGYAKGVLTQKLGPWRRPVAYLSKKLDPVAAGWPPCLRMVAAIAVLTK |
| | | DAGKLTMGQPLVILAPHAVEALVKQPPDRWLSNARMTHYQALLLDTDRVQF |
| | | GPVVALNPATLLPLPEEGLOHNCLDILAEAHGTRPDLTDQPLPDADHTWYTD |
| | | GSSLLQEGQRKAGAAVTTETEVIWAKALPAGTSAQRAELIALTQALKMAEGKK |
| | | LNVYTDSRYAFATAHIHGEIYRRRGWLTSEGKEIKNKDEILALLKALFLPKRLSII |
| | | HCPGHQKGHSAEARGNRMADQAARKAAITETPDTSTLLIENSSP |
| 3739 | -C-terminal NLS | SGGSKRTADGSEFEPKKKRKV |

TABLE 58
lists exemplary prime editor and its components

| SEQ | | |
|---|---|---|
| ID NO. | DESCRIPTION | SEQUENCE |
| 3741 | Exemplary prime | MKRTADGSEFESPKKKRKVDKKYSIGLDIGTNSVGWAVITDEYKVPSKKFKVL |
| | editor [NLS]- | GNTDRHSIKKNLIGALLFDSGETAEATRLKRTARRRYTRRKNRICYLQEIFSNE |
| | [Cas9((R220K) | MAKVDDSFFHRLEESFLVEEDKKHERHPIFGNIVDEVAYHEKYPTIYHLRKKL |
| | (R393K) (H839A)]- | VDSTDKADLRLIYLALAHMIKFRGHFLIEGDLNPDNSDVDKLFIQLVQTYNQL |
| | [linker]- | FEENPINASGVDAKAILSARLSKSRKLENLIAQLPGEKKNGLFGNLIALSLGLTP |
| | [MMLV_RT(D200N)(T | NFKSNFDLAEDAKLQLSKDTYDDDLDNLLAQIGDQYADLFLAAKNLSDAILL |
| | 330P)(L603W)(T306K) | SDILRVNTEITKAPLSASMIKRYDEHHQDLTLLKALVRQQLPEKYKEIFFDQSK |
| | (W313F)]-[NLS] | NGYAGYIDGGASQEEFYKFIKPILEKMDGTEELLVKLKREDLLRKORTFDNGS |
| | | IPHQIHLGELHAILRRQEDFYPFLKDNREKIEKILTFRIPYYVGPLARGNSRFA |
| | | WMTRKSEETITPWNFEEVVDKGASAQSFIERMTNFDKNLPNEKVLPKHSLL |
| | | YEYFTVYNELTKVKYVTEGMRKPAFLSGEQKKAIVDLLFKTNRKVTVKQLKE |
| | | DYFKKIECFDSVEISGVEDRFNASLGTYHDLLKIIKDKDFLDNEENEDILEDIVL |
| | | TLTLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVKK |
| | | MKNYWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINNYHH |
| | | AHDAYLNAVVGTALIKKYPKLESEFVYGDYKVYDVRKMIAKSEQEIGKATAK |
| | | YFFYSNIMNFFKTEITLANGEIRKRPLIETNGETGEIVWDKGRDFATVRKVLS |
| | | MPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKD |
| | | LIIKLPKYSLFELENGRKRMLASAGELQKGNELALPSKYVNFLYLASHYEKLKG |
| | | SPEDNEQKQLFVEQHKHYLDEIIEQISEFSKRVILADANLDKVLSAYNKHRDK |
| | | PIREQAENIIHLFTLTNLGAPAAFKYFDTTIDRKRYTSTKEVLDATLIHQSITGL |
| | | YETRIDLSQLGGDSGGSSGGSKRTADGSEFESPKKKRKVSGGSSGGSTLNIED |
| | | EYRLHETSKEPDVSLGSTWLSDFPQAWAETGGMGLAVRQAPLIIPLKATSTPV |
| | | SIKQYPMSQEARLGIKPHIQRLLDQGILVPCQSPWNTPLLPVKKPGTNDYRPV |
| | | QDLREVNKRVEDIHPTVPNPYNLLSGLPPSHQWYTVLDLKDAFFCLRLHPTSQ |
| | | PLFAFEWRDPEMGISGQLTWTRLPQGFKNSPTLFNEALHRDLADFRIQHPDLI |
| | | LLQYVDDLLLAATSELDCQQGTRALLQTLGNLGYRASAKKAQICQKQVKYLGY |
| | | LLKEGQRWLTEARKETVMGQPTPKTPRQLREFLGKAGFCRLFIPGFAEMAAP |
| | | LYPLTKPGTLFNWGPDQQKAYQEIKQALLTAPALGLPDLTKPFELFVDEKQGY |
| | | AKGVLTQKLGPWRRPVAYLSKKLDPVAAGWPPCLRMVAAIAVLTKDAGKLT |
| | | MGQPLVILAPHAVEALVKQPPDRWLSNARMTHYQALLLDTDRVQFGPVVAL |
| | | NPATLLPLPEEGLQHNCLDILAEAHGTRPDLTDQPLPDADHTWYTDGSSLLQE |
| | | GQRKAGAAVTTETEVIWAKALPAGTSAQRAELIALTQALKMAEGKKLNVYTD |
| | | SRYAFATAHIHGEIYRRRGWLTSEGKEIKNKDEILALLKALFLPKRLSIIHCPGHQ |
| | | KGHSAEARGNRMADQAARKAAITETPDTSTLLIENSSPSGGSKRTADGSEFES |
| | | PKKKRKVGSGPAAKRVKLD |
| | | KEY: |
| | | N-terminal bipartiteSV40NLS |
| | | CAS9(R221K N394K H840A) |
| | | SGGSx2-met-bpSV40NLS-SGGSx2 LINKER |
| | | M-MLV D200N T306K W313F T330P L603W REVERSE TRANSCRIPTASE |
| | | C-terminal linker-NLS1 |
| | | C-terminal linker-NLS2 |
| 3730 | -N-terminal | MKRTADGSEFESPKKKRKV |
| | bpSV40NLS | |
| 3696 | -CAS9 (R221K | DKKYSIGLDIGTNSVGWAVITDEYKVPSKKFKVLGNTDRHSIKKNLIGALLFDSG |
| | N394K H840A) | ETAEATRLKRTARRRYTRRKNRICYLQEIFSNEMAKVDDSFFHRLEESFLVEEDK |
| | | KHERHPIFGNIVDEVAYHEKYPTIYHLRKKLVDSTDKADLRLIYLALAHMIKFRG |
| | | HFLIEGDLNPDNSDVDKLFIQLVQTYNQLFEENPINASGVDAKAILSARLSKSRK |
| | | LENLIAQLPGEKKNGLFGNLIALSLGLTPNFKSNFDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLKREDLLRKQRTFDNGSIPHQIHLGELHAILRRQEDFYPFLKDNREKIE |
| | | KILTFRIPYYVGPLARGNSRFAWMTRKSEETITPWNFEEVVDKGASAQSFIER |
| | | MTNFDKNLPNEKVLPKHSLLYEYFTVYNELTKVKYVTEGMRKPAFLSGEQKKA |
| | | IVDLLFKTNRKVTVKQLKEDYFKKIECFDSVEISGVEDRFNASLGTYHDLLKIIKD |
| | | KDFLDNEENEDILEDIVLTLTLFEDREMIEERLKTYAHLFDDKVMKOLKRRRYT |
| | | GWGRLSRKLINGIRDKQSGKTILDFLKSDGFANRNFMQLIHDDSLTFKEDIQK |
| | | AQVSGQGDSLHEHIANLAGSPAIKKGILQTVKVVDELVKVMGRHKPENIVIEM |
| | | ARENQTTQKGQKNSRERMKRIEEGIKELGSQILKEHPVENTQLQNEKLYLYYL |
| | | QNGRDMYVDQELDINRLSDYDVDAIVPQSFLKDDSIDNKVLTRSDKNRGKSD |
| | | NVPSEEVVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDFQFYKV |
| | | REINNYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDS |
| | | PTVAYSVLVVAKVEKGKSKKLKSVKELLGITIMERSSFEKNPIDFLEAKGYKEVKK |
| | | DLIIKLPKYSLFELENGRKRMLASAGELQKGNELALPSKYVNFLYLASHYEKLKG |
| | | SPEDNEQKOLFVEQHKHYLDEIIEQISEFSKRVILADANLDKVLSAYNKHRDKPI |
| | | REQAENIIHLFTLTNLGAPAAFKYFDTTIDRKRYTSTKEVLDATLIHQSITGLYET |
| | | RIDLSQLGGD |
| 3758 | -SGGSx2- | SGGSSGGSKRTADGSEFESPKKKRKVSGGSSGGS |
| | bpSV40NLS-SGGSx2 | |
| | linker | |
| 3692 | -MMLV_RT D200N | TLNIEDEYRLHETSKEPDVSLGSTWLSDFPQAWAETGGMGLAVRQAPLIIPLK |
| | T330P L603W T306K | ATSTPVSIKQYPMSQEARLGIKPHIQRLLDQGILVPCQSPWNTPLLPVKKPGT |
| | W313F | NDYRPVQDLREVNKRVEDIHPTVPNPYNLLSGLPPSHQWYTVLDLKDAFFCLR |
| | | LHPTSQPLFAFEWRDPEMGISGQLTWTRLPQGFKNSPTLFNEALHRDLADFRI |
| | | QHPDLILLQYVDDLLLAATSELDCQQGTRALLQTLGNLGYRASAKKAQICQKQ |
| | | VKYLGYLLKEGQRWLTEARKETVMGQPTPKTPRQLREFLGKAGFCRLFIPGFA |
| | | EMAAPLYPLTKPGTLFNWGPDQQKAYQEIKQALLTAPALGLPDLTKPFELFVD |
| | | EKQGYAKGVLTQKLGPWRRPVAYLSKKLDPVAAGWPPCLRMVAAIAVLTKD |
| | | AGKLTMGQPLVILAPHAVEALVKQPPDRWLSNARMTHYQALLLDTDRVQFG |
| | | PVVALNPATLLPLPEEGLOHNCLDILAEAHGTRPDLTDQPLPDADHTWYTDG |
| | | SSLLQEGQRKAGAAVTTETEVIWAKALPAGTSAQRAELIALTQALKMAEGKKL |
| | | NVYTDSRYAFATAHIHGEIYRRRGWLTSEGKEIKNKDEILALLKALFLPKRLSIIH |
| | | CPGHQKGHSAEARGNRMADQAARKAAITETPDTSTLLIENSSP |
| 3759 | C-terminal linker-NLS | SGGSKRTADGSEFESPKKKRKV |
| 3760 | C-terminal linker- | GSGPAAKRVKLD |
| | NLS2 | |

### PEgRNA for Editing of FANCC Gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence can be referred to as an extension arm.

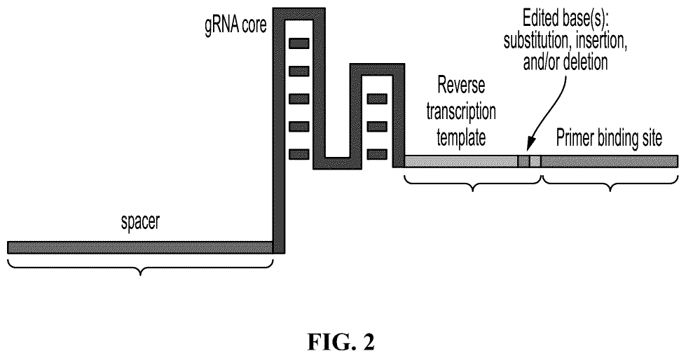

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene, e.g., an FANCC gene. In some embodiments, the editing template comprises substantial or partial complementarity to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing target, a PBS, a spacer, and a gRNA core.

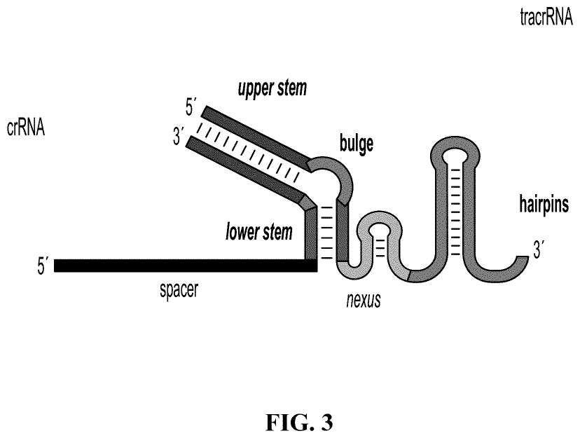

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA can comprise a first polynucleotide comprising the spacer and a first portion of a gRNA core comprising, which can be also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a spacer sequence comprises a region that has substantial complementarity to a search target sequence on the target strand of a double stranded target DNA, e.g. an FANCC gene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from about 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 16 to 22 nucleotides in length, e.g., about 16, 17, 18, 19, 20, 21, or 22 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

The extension arm of a PEgRNA can comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA can comprise a primer binding site sequence (PBS, or PBS sequence) that comprises complementarity to and can hybridize with a free 3′ end of a single stranded DNA in the target gene (e.g. FANCC gene) generated by nicking with a prime editor at the nick site on the PAM strand.

The length of the PBS sequence may vary depending on, e.g., the prime editor components, the search target sequence and other components of the PEgRNA.

In some embodiments, the PBS is about 3 to 19 nucleotides in length nucleotides in length. In some embodiments, the PBS is about 3 to 17 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is 8 to 17 nucleotides in length. In some embodiments, the PBS is 8 to 16 nucleotides in length. In some embodiments, the PBS is 8 to 15 nucleotides in length. In some embodiments, the PBS is 8 to 14 nucleotides in length. In some embodiments, the PBS is 8 to 13 nucleotides in length. In some embodiments, the PBS is 8 to 12 nucleotides in length. In some embodiments, the PBS is 8 to 11 nucleotides in length. In some embodiments, the PBS is 8 to 10 nucleotides in length. In some embodiments, the PBS is 8 or 9 nucleotides in length. In some embodiments, the PBS is 16 or 17 nucleotides in length. In some embodiments, the PBS is 15 to 17 nucleotides in length. In some embodiments, the PBS is 14 to 17 nucleotides in length. In some embodiments, the PBS is 13 to 17 nucleotides in length. In some embodiments, the PBS is 12 to 17 nucleotides in length. In some embodiments, the PBS is 11 to 17 nucleotides in length. In some embodiments, the PBS is 10 to 17 nucleotides in length. In some embodiments, the PBS is 9 to 17 nucleotides in length. In some embodiments, the PBS is about 7 to 15 nucleotides in length. In some embodiments, the PBS is 6, 7, 8, 9, 10, 11, 12, 13, 14, 15, 16, 17, 18, or 19 nucleotides in length. In some embodiments, the PBS is 8 to 14 nucleotides in length. For example, the PBS can be 8, 9, 10, 11, 12, 13, or 14 nucleotides in length. In some embodiments, the PBS is 11 or 12 nucleotides in length. In some embodiments, the PBS is 11 to 13 nucleotides in length. In some embodiments, the PBS is 11 to 14 nucleotides in length.

The PBS can be complementary or substantially complementary to a DNA sequence in the edit strand of the target gene. By annealing with the edit strand at a free hydroxy group, e.g. a free 3′ end generated by prime editor nicking, the PBS can initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., the FANCC gene). In some embodiments, the PBS is perfectly complementary, or 100% complementary, to a region of the edit strand of the target gene (e.g., the FANCC gene).

An extension arm of a PEgRNA may comprise an editing template that serves as a DNA synthesis template for the DNA polymerase in a prime editor during prime editing.

The length of an editing template can vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In some embodiments, the RTT is 10 to 110 nucleotides in length. In some embodiments, the RTT is 10 to 109, 10 to 108, 10 to 107, 10 to 106, 10 to 105, 10 to 104, 10 to 103, 10 to 102, or 10 to 101 nucleotides in length. In some embodiments, the RTT is at least 8 and no more than 50 nucleotides in length. In some embodiments, the RTT is at least 8 and no more than 25 nucleotides in length. In some embodiments, the RTT is about 10 to about 20 nucleotides in length. In some embodiments, the RTT is about 11, 12, 13, 14, 15, 16, 17, 18, or 19 nucleotides in length. In some embodiments, the RTT is 11 to 17 nucleotides in length. In some embodiments, the RTT is 12 to 17 nucleotides in length. In some embodiments, the RTT is 12 to 16 nucleotides in length. In some embodiments, the RTT is 13 to 17 nucleotides in length. In some embodiments, the RTT is 11, 12, 13, 14, 15, 16, or 17 nucleotides in length. In some embodiments the RTT is 12 nucleotides in length. In some embodiments the RTT is 16 nucleotides in length. In some embodiments the RTT is 17 nucleotides in length.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated in the target gene. In some embodiments, the editing template comprises a nucleotide sequence comprising about 85% to about 95% complementarity to an editing target sequence in the edit strand in the target gene (e.g. FANCC gene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the FANCC gene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 1, at least 2, at least 3, at least 4, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA can comprise one or more intended nucleotide edits, compared to the FANCC gene to be edited. Position of the intended nucleotide edit(s) relevant to other components of the PEgRNA, or to particular nucleotides (e.g., mutations) in the FANCC target gene can vary. In some embodiments, the nucleotide edit is in a region of the PEgRNA corresponding to or homologous to the protospacer sequence. In some embodiments, the nucleotide edit is in a region of the PEgRNA corresponding to a region of the FANCC gene outside of the protospacer sequence.

In some embodiments, the position of a nucleotide edit incorporation in the target gene may be upstream or downstream with respect to another sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene can be referred to based on position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template can be referred to by aligning the editing template with the partially complementary editing target sequence on the edit strand, and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand can be referred to as at positions +1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity can also be referred to as position 0 in the editing template, the nucleotides in the editing template corresponding to the nucleotides at positions +1, +2, +3, +4, . . . , +n on the PAM strand of the double stranded target DNA can also be referred to as at positions +1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions +1, +2, +3, +4, . . . , +n are 5′ of position 0 and positions −1, −2, −3, −4, . . . −n are 3′ of position 0 in the editing template. In some embodiments, an intended nucleotide edit is at position +n of the editing template relative to position 0. Accordingly, the intended nucleotide edit may be incorporated at position +n of the PAM strand of the double stranded target DNA (and subsequently, the target strand of the double stranded target DNA) by prime editing.

The corresponding positions of the intended nucleotide edit incorporated in the FANCC gene may also be referred to based on the nicking position generated by a prime editor based on sequence homology and complementarity. For example, in embodiments, the distance between the nucleotide edit to be incorporated into the FANCC gene and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand). As used herein, the distance between the nick site and the nucleotide edit, for example, where the nucleotide edit comprises an insertion or deletion, refers to the 3′ position of the nucleotide edit for a nick that creates a 3′ free end on the edit strand. For example, the nick-to-edit distance for a one-nucleotide insertion immediately downstream of the nick site is 1. In some embodiments, the nick-to-edit distance is from 1 to 150. In some embodiments, the nick-to-edit distance is from 1 to 10, from 10 to 20, from 20 to 30, from 30 to 40, from 40 to 50, from 50 to 60, from 60 to 70, from 70 to 80, from 80 to 90, from 90 to 100, from 100 to 110, from 110 to 120, from 120 to 130, from 130 to 140, or from 140 to 150. In some embodiments, the nick-to-edit distance is 1, 2, or 3. For a PEgRNA that can complex with a prime editor having a Cas9 nickase, e.g., a SpCas9 nickase, a nick-to-edit distance of 3 or less indicates that incorporation of the edit encoded by the RTT of the PEgRNA can alter the protospacer sequence that corresponds to the spacer sequence of the PEgRNA. Such edits may be referred to as protospacer edits. Without wishing to be bound by theory, protospacer edits may prevent the Cas9 nickase from re-nicking the edit strand, thereby improving prime editing efficiency or reduce indel formation.

The RTT length and the nick-to-edit distance relate to the length of the portion of the RTT that is upstream of (i.e. 5′ to) the 5′-most edit in the RTT and is complementary to the edit strand. In some embodiments, the editing template comprises at least 4 contiguous nucleotides of complementarity with the edit strand wherein the at least 4 nucleotides contiguous are located upstream of the 5′ most edit in the editing template. In some embodiments, the editing template comprises at least 5, 6, 7, 8, 9, 10, 11, 12, 13, 14, 15, 16, 17, 18, 19, or more contiguous nucleotides of complementarity with the edit strand wherein the at least 5, 6, 7, 8, 9, 10, 11, 12, 13, 14, 15, 16, 17, 18, 19, or more contiguous nucleotides are located upstream of the 5′ most edit in the editing template. In some embodiments, the editing template comprises 20-25, 25-30, 30-35, 35-40, 45-45, or 45-50 contiguous nucleotides of complementarity with the edit strand wherein the 20-25, 25-30, 30-35, 35-40, 45-45, or 45-50 or more contiguous nucleotides are located upstream of the 5′ most edit in the editing template. In some embodiments, the editing template comprises 9-14 contiguous nucleotides of complementarity with the edit strand wherein the 9-14 contiguous nucleotides are located upstream of the 5′ most edit in the editing template. In some embodiments, the editing template comprises 6-10 contiguous nucleotides of complementarity with the edit strand wherein the 6-10 contiguous nucleotides are located upstream of the 5′ most edit in the editing template. In some embodiments, the editing template comprises 10 contiguous nucleotides of complementarity with the edit strand wherein the 10 contiguous nucleotides are located upstream of the 5′ most edit in the editing template. In some embodiments, the editing template comprises 9 contiguous nucleotides of complementarity with the edit strand wherein the 9 contiguous nucleotides are located upstream of the 5′ most edit in the editing template.

When referred to within the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the PBS. In some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the PBS.

In some embodiments, the editing template can comprise a second edit relative to a target sequence. The second edit can be designed to mutate or edit a PAM sequence such that a corresponding nucleic acid guided nuclease or CRISPR nuclease is no longer able to cleave the target sequence (such edits referred to as “PAM silencing edits).

Without wishing to be bound by any particular theory, PAM silencing edits may prevent the Cas, e.g., Cas9, nickase, from re-nicking the edit strand before the edit is incorporated in the target strand, therefore improving prime editing efficiency. In some embodiments, a PAM silencing edit is a synonymous edit that does not alter the amino acid sequence encoded by the FANCC gene after incorporation of the edit. In some embodiments, a PAM silencing edit is at a position corresponding to a coding region, e.g., an exon, of a FANCC gene. In some embodiments, a PAM silencing edit is at a position corresponding to a non-coding region, e.g., an intron, of a FANCC gene. In some embodiments, the edits in an intron of a FANCC gene is not at a position that corresponds to intron-exon junction and the edit does not affect transcript splicing.

In some embodiments, the length of the editing template is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides longer than the nick to edit distance. In some embodiments, the length of the editing template is at least 4 nucleotides longer than the nick to edit distance. In some embodiments, for example, the nick to edit distance is 8 nucleotides, and the editing template is 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, or 10 to 80 nucleotides in length. In some embodiments, the nick to edit distance is 22 nucleotides, and the editing template is 24 to 28, 24 to 30, 24 to 32, 24 to 34, 24 to 36, 24 to 37, 24 to 38, 24 to 40, 24 to 45, 24 to 50, 24 to 55, 24 to 60, 24 to 65, 24 to 70, 24 to 75, 24 to 80, 24 to 85, 24 to 90, 24 to 95, 24 to 100, 24 to 105, 24 to 100, 24 to 105, or 24 to 110 nucleotides in length.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g. by reverse transcription) to replace an editing target sequence in the target gene. In some embodiments, the editing target sequence in the edit strand of the target gene is replaced by the newly synthesized strand, and the nucleotide edit(s) are incorporated in the region of the target gene. In some embodiments, the target gene is an FANCC gene. In some embodiments, the editing template of the PEgRNA encodes a newly synthesized single stranded DNA that comprises a wild type FANCC gene sequence. In some embodiments, the newly synthesized DNA strand replaces the editing target sequence in the target FANCC gene, wherein the editing target sequence (or the endogenous sequence complementary to the editing target sequence on the target strand of the FANCC gene) comprises a mutation or a nucleotide alteration compared to a wild type FANCC gene. In some embodiments, the mutation is associated with Fanconi anemia; FA-C.

In some embodiments, the editing target sequence comprises a mutation in an intron of the FANCC gene as compared to a wild type FANCC gene. In some embodiments, the editing target sequence comprises a mutation in an intron of the FANCC gene that results in altered or aberrant splicing of a transcript encoded by the FANCC gene compared to a transcript encoded by a wild type FANCC gene.

In some embodiments, the editing target sequence comprises a mutation that is located between positions 95,171,933 and 95,172,133 of human chromosome 9. In some embodiments, the editing target sequence comprises a mutation in intron 4 of the FANCC gene compared to a wild type FANCC gene. In some embodiments, the editing target sequence comprises a mutation that encodes a nucleotide substitution compared to a wild type FANCC gene as set forth in SEQ ID NO: 3809. In some embodiments, the editing target sequence comprises a nucleotide transversion relative to a wild type FANCC gene set forth as SEQ ID NO: 3809. In some embodiments, the editing target sequence comprises an A to T transversion at position 95,172,033 in human chromosome 9 (a c.456+4A->T (IVS4+4A>T) mutation) as compared to a wild type FANCC gene.

In some embodiments, the editing target sequence comprises a mutation in an exon of the FANCC gene as compared to a wild type FANCC gene. In some embodiments, the editing target sequence comprises a mutation in exon 1 of the FANCC gene as compared to a wild type FANCC gene.

In some embodiments, the editing target sequence comprises a mutation that is located between positions 95,249,125 and 95,249,325 of human chromosome 9. In some embodiments, the editing target sequence comprises a mutation that results in a frameshift in a transcript encoded by the FANCC gene as compared to a transcript encoded by a wild type FANCC gene. In some embodiments, the editing target sequence comprises a mutation that is a deletion compared to a wild type FANCC gene set forth as SEQ ID NO: 3809. In some embodiments, the editing target sequence comprises a deletion of a nucleotide guanine at position 95,249,225 in human chromosome 9 (a c.67del (322delG) mutation) as compared to a wild type FANCC gene.

In some embodiments, the editing template comprises one or more intended nucleotide edits compared to the sequence on the target strand of the FANCC gene that is complementary to the editing target sequence. In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits.

In some embodiments, incorporation of the one or more intended nucleotide edits corrects the mutation in the editing target sequence to wild type nucleotides at corresponding positions in the FANCC gene. As used herein, “correcting” a mutation means restoring a wild type sequence at the place of the mutation in the double stranded target DNA, e.g. target gene, by prime editing. In some embodiments, the editing template comprises and/or encodes a wild type FANCC gene sequence.

In some embodiments, incorporation of the one or more intended nucleotide edits does not correct the mutation in the editing target sequence to wild type sequence, but allows for expression of a functional FANCC protein encoded by the FANCC gene.

In some embodiments, the editing template comprises one or more intended nucleotide edits compared to the sequence on the target strand of the FANCC gene that is complementary to the editing target sequence, wherein the one or more intended nucleotide edits is a single nucleotide substitution. In some embodiments, the intended nucleotide edit in the editing template comprises an A to T nucleotide substitution compared to the sequence on the target strand of the FANCC gene that is complementary to the editing target sequence (that is, the single stranded DNA encoded by the editing template comprises a T to A nucleotide substitution compared to the editing target sequence) at a position corresponding to position 95,172,033 of human chromosome 9, wherein the editing target sequence is on the sense strand of the FANCC gene. In some embodiments, the intended nucleotide edit in the editing template comprises a T to A nucleotide substitution compared to the sequence on the target strand of the FANCC gene that is complementary to the editing target sequence (that is, the single stranded DNA encoded by the editing template comprises an A to T nucleotide substitution compared to the editing target sequence) at a position corresponding to position 95,172,033 of human chromosome 9, wherein the editing target sequence is on the antisense strand of the FANCC gene.

In some embodiments, the editing template comprises one or more intended nucleotide edits compared to the sequence on the target strand of the FANCC gene that is complementary to the editing target sequence, wherein the one or more intended nucleotide edits is a single nucleotide insertion. In some embodiments, the intended nucleotide edit in the editing template comprises an insertion of nucleotide Cytidine compared to the sequence on the target strand of the FANCC gene that is complementary to the editing target sequence (that is, the single stranded DNA encoded by the editing template comprises an insertion of nucleotide Guanine compared to the editing target sequence) at a position corresponding to position 95,249,225 of human chromosome 9, wherein the editing target sequence is on the sense strand of the FANCC gene. In some embodiments, the intended nucleotide edit in the editing template comprises an insertion of nucleotide Guanine compared to the sequence on the target strand of the FANCC gene that is complementary to the editing target sequence (that is, the single stranded DNA encoded by the editing template comprises an insertion of nucleotide Cytidine compared to the editing target sequence) at a position corresponding to position 95,249,225 of human chromosome 9, wherein the editing target sequence is on the antisense strand of the FANCC gene.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-U pairs, for example, a GUUUU-AAAAC pairing element. In some embodiments, a prime editing system comprises a prime editor and a PEgRNA, wherein the prime editor comprises a SpCas9 nickase variant thereof, and the gRNA core comprises the sequence provided in SEQ ID NOs: 3666, 3667, or 3668.

The gRNA core sequences below are annotated with SEQ ID NO as required by ST.26 standard. Although all the sequences provided in Table 62 are RNA sequences, “T” is used instead of a “U” in the sequences for consistency with the ST.26 standard.

(SEQ ID NO: 3666)
GTTTTAGAGCTAGAAATAGCAAGTTAAAATAAGGCTAGTCCGTTATCAA
CTTGAAAAAGTGGCACCGAGTCGGTGC;
(SEQ ID NO: 3667)
GTTTGAGAGCTAGAAATAGCAAGTTTAAATAAGGCTAGTCCGTTATCAA
CTTGAAAAAGTGGGACCGAGTCGGTCC,
or
(SEQ ID NO: 3668)
GTTTAAGAGCTATGCTGGAAACAGCATAGCAAGTTTAAATAAGGCTAGT
CCGTTATCAACTTGAAAAAGTGGCACCGAGTCGGTGC.

In some embodiments, the gRNA core comprises the sequence

(SEQ ID NO: 3666)
GTTTTAGAGCTAGAAATAGCAAGTTAAAATAAGGCTAGTCCGTTATCAA
CTTGAAAAAGTGGCACCGAGTCGGTGC.

Any gRNA core sequences known in the art are also contemplated in the prime editing compositions described herein.

The gRNA core of a PEgRNA or ngRNA can be any gRNA scaffold sequence that is capable of interacting with a Cas protein that recognizes the corresponding PAM of the PEgRNA or ngRNA. In some embodiments, gRNA core of a PEgRNA or a ngRNA comprises a nucleic acid sequence selected from SEQ ID Nos: 3666-3670 or 3796-3800.

A PEgRNA can also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers can be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). In some embodiments, a ngRNA comprises a spacer (referred to as a ngRNA spacer or ng spacer) and a gRNA core, wherein the spacer of the ngRNA comprises a region of complementarity to the edit strand, and wherein the gRNA core can interact with a Cas, e.g., Cas9, of a prime editor. Without wishing to be bound by any particular theory, an ngRNA may bind to the edit strand and direct the Cas nickase to generate a nick on the non-edit strand (or target strand). In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

A prime editing system comprising a PEgRNA (or one or more polynucleotide encoding the PEgRNA) and a prime editor protein (or one or more polynucleotides encoding the prime editor), may be referred to as a PE2 prime editing system and the corresponding editing approach referred to as PE2 approach or PE2 strategy. A PE2 system does not contain a ngRNA. A prime editing system comprising a PEgRNA (or one or more polynucleotide encoding the PEgRNA), a prime editor protein (or one or more polynucleotides encoding the prime editor), and a ngRNA (or one or more polynucleotides encoding the ngRNA) may be referred to as a “PE3” prime editing system. In some embodiments, an ngRNA spacer sequence is complementary to a portion of the edit strand that includes the intended nucleotide edit, and may hybridize with the edit strand only after the edit has been incorporated on the edit strand. Such ngRNA may be referred to a “PE3b” ngRNA, and the prime editing system a PE3b prime editing system.

In some embodiments, a PEgRNA or a nick guide RNA (ngRNA) can be chemically synthesized, or can be assembled or cloned and transcribed from a DNA sequence, e.g., a plasmid DNA sequence, or by any RNA oligonucleotide synthesis method known in the art. In some embodiments, DNA sequence that encodes a PEgRNA (or ngRNA) can be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) can be designed to append a nucleotide G at the 5′ end. Accordingly, in some embodiments, the PEgRNA (or nick guide RNA) can comprise an appended nucleotide G at the 5′ end. In some embodiments, a DNA sequence that encodes a PEgRNA (or nick guide RNA) can be designed to append a sequence that enhances transcription, e.g., a Kozak sequence, at the 5′ end. In some embodiments, a DNA sequence that encodes a PEgRNA (or nick guide RNA) can be designed to append the sequence CACC or CCACC at the 5′ end. Accordingly, in some embodiments, the PEgRNA (or nick guide RNA) can comprise an appended sequence CACC or CCACC at the 5′ end. In some embodiments, a DNA sequence that encodes a PEgRNA (or nick guide RNA) can be designed to append the sequence TTT, TTTT. TTTTT, TTTTTT, or TTTTTTT at the 3′ end. Accordingly, in some embodiments, the PEgRNA (or nick guide RNA) can comprise an appended sequence UUU, UUUU, UUUUU, UUUUUU, or UUUUUUU at the 3′ end.

In some embodiments, a PEgRNA or a ngRNA comprises terminal or end adaptation sequences. In some embodiments, a PEgRNA or a ngRNA comprises the sequence UUUU (sequence number 3807) at the 3′ end. In some embodiments, a PEgRNA or a ngRNA comprises the sequence UUUUUUU (sequence number 3808) at the 3′ end. In some embodiments, a PEgRNA or a ngRNA comprises a 3′ terminator sequence (e.g., UUUU) at the 3′ end. In some embodiments, a PEgRNA or a ngRNA comprises a transcription adaptation sequence (e.g., UUUUUUU) at the 3′ end.

As shown in Tables 1-52 herein, in some embodiments, a PEgRNA or a ngRNA comprises the sequence TTTT (sequence number 3807) at the 3′ end. In some embodiments, a PEgRNA or a ngRNA comprises the sequence TTTTTTT (sequence number 3808) at the 3′ end. In some embodiments, a PEgRNA or a ngRNA comprises a 3′ terminator sequence (e.g., TTTT; sequence number 3807) at the 3′ end. In some embodiments, a PEgRNA or a ngRNA comprises a transcription adaptation sequence (e.g., TTTTTTT sequence number 3808) at the 3′ end. The sequences in sequence number 3807 and sequence number 3808 are annotated with a sequence number as required by ST.26 standard. Although the sequences set forth in sequence number 3807 and sequence number 3808 are RNA sequences, “T” is used instead of a “U” in the sequences for consistency with the ST.26 standard.

In some embodiments, a PEgRNA or ngRNA may include a modifying sequence at the 3′ end having the sequence AACAUUGACGCGUCUCUACGUGGGGGCGCG (SEQ ID NO: 3761).

In some embodiments, a PEgRNA or ngRNA may include a modifying sequence at the 3′ end having the sequence AACAUUGA (sequence number 3811).

In some embodiments, a PEgRNA or ngRNA can include a linker sequence comprising the sequence AACAUUGA (sequence number 3811). In Tables 1-52, this linker sequence is set forth as AACATTGA with “T” instead of “U”, for consistency with the ST.26 standard.

In some embodiments, a PEgRNA or ngRNA can include a hairpin sequence comprising CGCGTCTCTACGTGGGGGCGCG (SEQ ID NO: 3591). In some embodiments, a PEgRNA or ngRNA comprises a 3′ hairpin sequence comprising SEQ ID NO: 3591. In SEQ ID NO: 3591, “T” is used instead of “U”, for consistency with the ST.26 standard.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. In some embodiments, such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

A ngRNA protospacer may be in close proximity to the PEgRNA spacer, or may be upstream or downstream of the PEgRNA spacer. In some embodiments, the distance generated by the PEgRNA nick site and the ngRNA nick site (referred to as the nick-to-nick distance) is about 3 to about 100 nucleotides. In some embodiments, the distance generated by the PEgRNA nick site and the ngRNA nick site (referred to as the nick-to-nick distance) is about 4-90, 4-80, 4-70, 4-60, 4-50, 4-40, 4-30, 4-20, or 4-10 nucleotides.

In some embodiments, the distance generated by the PEgRNA nick site and the ngRNA nick site (referred to as the nick-to-nick distance) is about 10-20, 20-30, 30-40, 40-50, 50-60, 60-70, 70-80, 80-90, or 90-100 nucleotides. In some embodiments, the nick-to-nick distance is about 4-88 nucleotides. In some embodiments, the nick-to-nick distance is about 4-72 nucleotides. In some embodiments, the nick-to-nick distance is about 4-61 nucleotides. In some embodiments, the nick-to-nick distance is about 61-72 nucleotides. In some embodiments, the nick-to-nick distance is about 61-88 nucleotides. In some embodiments, the nick-to-nick distance is about 72-88 nucleotides. In some embodiments, the nick-to-nick distance is about 4-7 nucleotides. In some embodiments, the nick-to-nick distance is 4, 5, 6, or 7 nucleotides. In some embodiments, the nick-to-nick distance is about 41-96 nucleotides. In some embodiments, the nick-to-nick distance is about 41-82 nucleotides. In some embodiments, the nick-to-nick distance is about 41-44 nucleotides. In some embodiments, the nick-to-nick distance is about 44-82 nucleotides. In some embodiments, the nick-to-nick distance is about 44-96 nucleotides. In some embodiments, the nick-to-nick distance is about 82-96 nucleotides. In some embodiments, the nick-to-nick distance is 41, 44, 82, or 96 nucleotides. In some embodiments, the intended nucleotide edit is incorporated within about 1-10 nucleotides of the position corresponding to the PAM of the ng search target sequence.

In some embodiments, a PEgRNA (or ngRNA) comprises an additional secondary structure at the 5′ end. In some embodiments, a PEgRNA (or ngRNA) comprises an additional secondary structure at the 3′ end.

In some embodiments, the secondary structure comprises a pseudoknot. In some embodiments, the secondary structure comprises a pseudoknot derived from a virus. In some embodiments, the secondary structure comprises a pseudoknot of a Moloney murine leukemia virus (M-MLV) genome (a mpknot). In some embodiments, the secondary structure comprises a nucleotide sequence selected from the group consisting of
GGGUCAGGAGCCCCCCCCCUGAACCCAGGAUAACCCUCAAAGUCGGGGGGCAACC (SEQ ID NO: 3762),
GUCAGGGUCAGGAGCCCCCCCCCUGAACCCAGGAUAACCCUCAAAGUCGGGGGGCAACCC (SEQ ID NO: 3763),
GGGUCAGGAGCCCCCCCCCUGAACCCAGGAAAACCCUCAAAGUCGGGGGGCAACCC (SEQ ID NO: 3764),
GGGUCAGGAGCCCCCCCCCUGCACCCAGGAAAACCCUCAAAGUCGGGGGGCAACCC (SEQ ID NO: 3765),
GGGUCAGGAGCCCCCCCCCUGCACCCAGGAUAACCCUCAAAGUCGGGGGGCAACCC (SEQ ID NO: 3766),
GUCAGGGUCAGGAGCCCCCCCCCUGAACCCAGGAAAACCCUCAAAGUCGGGGGGCAACCC (SEQ ID NO: 3767),
GUCAGGGUCAGGAGCCCCCCCCCUGCACCCAGGAAAACCCUCAAAGUCGGGGGGCAACCC (SEQ ID NO: 3768), and
GGGUCAGGAGCCCCCCCCCUGAACCCAGGAUAACCCUCAAAGUCGGGGGGC (SEQ ID NO: 3769), or a nucleotide sequence having at least 80%, 85%, 90%, 95%, 96%, 97%, 98%, or 99% sequence identity therewith. In some embodiments, the secondary structure comprises a nucleotide sequence of GGGUCAGGAGCCCCCCCCCUGAACCCAGGAUAACCCUCAAAGUCGGGGGGC (SEQ ID NO: 3769), or a nucleotide sequence having at least 80%, 85%, 90%, 95%, 96%, 97%, 98%, or 99% sequence identity therewith.

In some embodiments, the secondary structure comprises a quadruplex. In some embodiments, the secondary structure comprises a G-quadruplex. In some embodiments, the secondary structure comprises a nucleotide sequence selected from the group consisting of gq2(UGGUGGUGGUGGU) (SEQ ID NO: 3770), stk40(GGGACAGGGCAGGGACAGGG) (SEQ ID NO: 3771), apc2(GGGUCCGGGUCUGGGUCUGGG) (SEQ ID NO: 3772), stard3(GGGCAGGGUCUGGGCUGGG) (SEQ ID NO: 3773), tnsl(GGGCUGGGAUGGGAAAGGG) (SEQ ID NO: 3774), ceacam4(GGGCUCUGGGUGGGCCGGG) (SEQ ID NO: 3775), erc1(GGGCUGGGCUGGGCAGGG) (SEQ ID NO: 3776), pitpnm3(GGGUGGGCUGGGAAGGG) (SEQ ID NO: 3777), rlf(GGGAGGGAGGGCUAGGG) (SEQ ID NO: 3778), ube3c(GGGCAGGGCUGGGAGGG) (SEQ ID NO: 3779), tafl5(GGGUGGGAGGGCUGGG) (SEQ ID NO: 3780), and xrnl(GCGUAACCUCCAUCCGAGUUGCAAGAGAGGGAAACGCAGUCUC) (SEQ ID NO: 3781), or a nucleotide sequence having at least 80%, 85%, 90%, 95%, 96%, 97%, 98%, or 99% sequence identity therewith.

In some embodiments, the secondary structure comprises a P4-P6 domain of a Group I intron. In some embodiments, the secondary structure comprises the nucleotide sequence of GGAAUUGCGGGAAAGGGGUCAACAGCCGUUCAGUACCAAGUCUCAGGGGAAACUUUGAG AUGGCCUUGCAAAGGGUAUGGUAAUAAGCUGACGGACAUGGUCCUAACCACGCAGCCAAG UCCUAAGUCAACAGAUCUUCUGUUGAUAUGGAUGCAGUUCA (SEQ ID NO: 3782), or a nucleotide sequence having at least 80%, 85%, 90%, 95%, 96%, 97%, 98%, or 99% sequence identity therewith.

In some embodiments, the secondary structure comprises a riboswitch aptamer. In some embodiments, the secondary structure comprises a riboswitch aptamer derived from a prequeosine-1 riboswitch aptamer. In some embodiments, the secondary structure comprises a modified prequeosine-1 riboswitch aptamer. In some embodiments, the secondary structure comprises a nucleotide sequence selected from the group consisting of UUGACGCGGUUCUAUCUAGUUACGCGUUAAACCAACUAGAAA (SEQ ID NO: 3783), UUGACGCGGUUCUAUCUACUUACGCGUUAAACCAACUAGAAA (SEQ ID NO: 3784), CGCGAGUCUAGGGGAUAACGCGUUAAACUUCCUAGAAGGCGGUU (SEQ ID NO: 3785), CGCGGAUCUAGAUUGUAACGCGUUAAACCAUCUAGAAGGCGGUU (SEQ ID NO: 3786), CGCGUCGCUACCGCCCGGCGCGUUAAACACACUAGAAGGCGGUU (SEQ ID NO: 3787), and CGCGGUUCUAUCUAGUUACGCGUUAAACCAACUAGAA (SEQ ID NO: 3788), or a nucleotide sequence having at least 80%, 85%, 90%, 95%, 96%, 97%, 98%, or 99% sequence identity therewith. In some embodiments, the secondary structure comprises a nucleotide sequence selected from the group consisting of UUGACGCGGUUCUAUCUAGUUACGCGUUAAACCAACUAGAAA (SEQ ID NO: 3783), CGCGAGUCUAGGGGAUAACGCGUUAAACUUCCUAGAAGGCGGUU (SEQ ID NO: 3785), CGCGGAUCUAGAUUGUAACGCGUUAAACCAUCUAGAAGGCGGUU (SEQ ID NO: 3786), CGCGUCGCUACCGCCCGGCGCGUUAAACACACUAGAAGGCGGUU (SEQ ID NO: 3787), and CGCGGUUCUAUCUAGUUACGCGUUAAACCAACUAGAA (SEQ ID NO: 3788), or a nucleotide sequence having at least 80%, 85%, 90%, 95%, 96%, 97%, 98%, or 99% sequence identity therewith. In some embodiments, the secondary structure comprises a nucleotide sequence CGCGGUUCUAUCUAGUUACGCGUUAAACCAACUAGAA (SEQ ID NO: 3788), or a nucleotide sequence having at least 80%, 85%, 90%, 95%, 96%, 97%, 98%, or 99% sequence identity therewith.

In some embodiments, a PEgRNA comprises a linker. In some embodiments, the secondary structure or a 3′ motif is linked to one or more other component of a PEgRNA via a linker. For example, in some embodiments, the secondary structure is at the 3′ end of the PEgRNA (e.g., a RTT, or a PBS) and is linked to the 3′ end of a PBS via a linker. For example, in some embodiments, a 3′ motif is at the 3′ end of the PEgRNA and is linked to the 3′ end of a PEgRNA (e.g., a RTT or a PBS) via a linker. In some embodiments, the secondary structure or a 5′ motif is at the 5′ end of the PEgRNA and is linked to the 5′ end of a spacer via a linker. In some embodiments, the linker is a nucleotide linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the linker is 5 to 10 nucleotides in length. In some embodiments, the linker is 10 to 20 nucleotides in length. In some embodiments, the linker is 15 to 25 nucleotides in length. In some embodiments, the linker is 8 nucleotides in length.

In some embodiments, the linker is designed to minimize base pairing between the linker and another component of the PEgRNA. In some embodiments, the linker is designed to minimize base pairing between the linker and the spacer. In some embodiments, the linker is designed to minimize base pairing between the linker and the PBS. In some embodiments, the linker is designed to minimize base pairing between the linker and the editing template. In some embodiments, the linker is designed to minimize base pairing between the linker and the sequence of the RNA secondary structure. In some embodiments, the linker is optimized to minimize base pairing between the linker and another component of the PEgRNA, in order of the following priority: spacer, PBS, editing template and then scaffold. In some embodiments, base paring probability is calculated using ViennaRNA 2.0,as described in Lorenz, R. et al. ViennaRNA package 2.0. Algorithms Mol. Biol. 6, incorporated by reference in its entirety herein, under standard parameters (37° C., 1 M NaCl, 0.05 M MgCl2).

In some embodiments, the PEgRNA comprises a RNA secondary structure and/or a linker disclosed in Nelson et al. Engineered pegRNAs improve prime editing efficiency. Nat Biotechnol. (2021), the entirety of which is incorporated herein by reference.

In some embodiments, a PEgRNA is transcribed from a nucleotide encoding the PEgRNA, for example, a DNA plasmid encoding the PEgRNA. In some embodiments, the PEgRNA comprises a self-cleaving element. In some embodiments, the self-cleaving element improves transcription and/or processing of the PEgRNA when transcribed form the nucleotide encoding the PEgRNA. In some embodiments, the PEgRNA comprises a hairpin or a RNA quadruplex. In some embodiments, the PEgRNA comprises a self-cleaving ribozyme element, for example, a hammerhead, a pistol, a hatchet, a hairpin, a VS, a twister, or a twister sister ribozyme. In some embodiments, the PEgRNA comprises a HDV ribozyme. In some embodiments, the PEgRNA comprises a hairpin recognized by Csy4. In some embodiments, the PEgRNA comprises an ENE motif. In some embodiments, the PEgRNA comprises an element for nuclear expression (ENE) from MALATI Inc RNA. In some embodiments, the PEgRNA comprises an ENE element from Kaposi's sarcoma-associated herpesvirus (KSHV). In some embodiments, the PEgRNA comprises a 3′ box of a U1 snRNA. In some embodiments, the PEgRNA forms a circular RNA.

In some embodiments, the PEgRNA comprises a RNA secondary structure or a motif that improves binding to the DNA-RNA duple or enhances PEgRNA activity. In some embodiments, the PEgRNA comprises a sequence derived from a native nucleotide element involved in reverse transcription, e.g., initiation of retroviral transcription. In some embodiments, the PEgRNA comprises a sequence of, or derived from, a primer binding site of a substrate of a reverse transcriptase, a polypurine tract (PPT), or a kissing loop. In some embodiments, the PEgRNA comprises a dimerization motif, a kissing loop, or a GNRA tetraloop—tetraloop receptor pair that results in circularization of the PEgRNA. In some embodiments, the PEgRNA comprises a RNA secondary structure of a motif that results in physical separation of the spacer and the PBS of the PEgRNA, thereby prevents occlusion of the spacer and improves PEgRNA activity. In some embodiments, the PEgRNA comprises a secondary structure or motif, e.g., a 5′ or 3′ extension in the spacer region that form a toehold or hairpin, wherein the secondary structure or motif competes favorably against annealing between the spacer and the PBS of the PEgRNA, thereby prevents occlusion of the spacer and improves PEgRNA activity.

In some embodiments, a PEgRNA comprises the sequence GGCCGGCAUGGUCCCAGCCUCCUCGCUGGCGCCGGCUGGGCAACAUGCUUCGGCAUGGCGA AUGGGAC (SEQ ID NO: 3789) at the 3′ end. In some embodiments, a PEgRNA comprises the structure [spacer]-[gRNA core]-[editing template]-[PBS]-GGCCGGCAUGGUCCCAGCCUCCUCGCUGGCGCCGGCUGGGCAACAUGCUUCGGCAUGGCGA AUGGGAC (SEQ ID NO: 3789), or [spacer]-[gRNA core]-[editing template]-[PBS]-GGCCGGCAUGGUCCCAGCCUCCUCGCUGGCGCCGGCUGGGCAACAUGCUUCGGCAUGGCGA AUGGGAC-(U)n (SEQ ID NO: 3801), wherein n is an integer between 3 and 7. The structure derived from hepatitis D virus (HDV) is italicized.

In some embodiments, the PEgRNA comprises the sequence GGUGGGAGACGUCCCACC (SEQ ID NO: 3790) at the 5′ end and/or the sequence UGGGAGACGUCCCACC (SEQ ID NO: 3802) at the 3′ end. In some embodiments, the PEgRNA comprises the following structure (M-MLV kissing loop): GGUGGGAGACGUCCCACC (SEQ ID NO: 3790)-[spacer]-[gRNA core]-[editing template]-[PBS]-UGGGAGACGUCCCACC (SEQ ID NO: 3802), or GGUGGGAGACGUCCCACC (SEQ ID NO: 3790)-[spacer]-[gRNA core]-[editing template]-[PBS]-UGGGAGACGUCCCACC-(U)n (SEQ ID NO: 3803), wherein n is an integer between 3 and 7. The kissing loop structure is italicized.

In some embodiments, the PEgRNA comprises the sequence GAGCAGCAUGGCGUCGCUGCUCAC (SEQ ID NO: 3791) at the 5′ end and/or the sequence CCAUCAGUUGACACCCUGAGG (SEQ ID NO: 3792) at the 3′ end. In some embodiments, the PEgRNA comprises the following structure (VS ribozyme kissing loop):
GAGCAGCAUGGCGUCGCUGCUCAC (SEQ ID NO: 3791)-[spacer]-[gRNA core]-[editing template]-[PBS]-CCAUCAGUUGACACCCUGAGG (SEQ ID NO: 3792), or GAGCAGCAUGGCGUCGCUGCUCAC (SEQ ID NO: 3791)-[spacer]-[gRNA core]-[editing template]-[PBS]-CCAUCAGUUGACACCCUGAGG-(U)n (SEQ ID NO: 3804), wherein n is an integer between 3 and 7. (VS ribozyme kissing loop)

In some embodiments, the PEgRNA comprises the sequence GCAGACCUAAGUGGUGACAUAUGGUCUG (SEQ ID NO: 3793) at the 5′ end and/or the sequence CAUGCGAUUAGAAAUAAUCGCAUG (SEQ ID NO: 3794) at the 3′ end. In some embodiments, the PEgRNA comprises the following structure (tetraloop and receptor): GCAGACCUAAGUGGUGACAUAUGGUCUG (SEQ ID NO: 3793)-[spacer]-[gRNA core]-[editing template]-[PBS]-CAUGCGAUUAGAAAUAAUCGCAUG (SEQ ID NO: 3794), or GCAGACCUAAGUGGUGACAUAUGGUCUG (SEQ ID NO: 3793)-[spacer]-[gRNA core]-[editing template]-[PBS]-CAUGCGAUUAGAAAUAAUCGCAUG-(U)n (SEQ ID NO: 3805), wherein n is an integer between 3 and 7. The tetraloop/tetraloop receptor structure is italicized.

In some embodiments, the PEgRNA comprises the sequence

(SEQ ID NO: 3789)
GGCCGGCAUGGUCCCAGCCUCCUCGCUGGCGCCGGCUGGGCAACAUGCU
UCGGCAUGGCGAAUGGGAC
or
(SEQ ID NO: 3795)
UCUGCCAUCAAAGCUGCGACCGUGCUCAGUCUGGUGGGAGACGUCCCAC
CGGCCGGCAUGGUCCCAGCCUCCUCGCUGGCGCCGGCUGGGCAACAUGC
UUCGGCAUGGCGAAUGGGAC.

In some embodiments, a PEgRNA comprises a gRNA core that comprises a modified direct repeat compared to the sequence of a naturally occurring CRISPR-Cas guide RNA scaffold, for example, a Cas9 gRNA scaffold. In some embodiments, the PEgRNA comprises a “flip and extension (F+E)” gRNA core, wherein one or more base pairs in a direct repeat is modified. In some embodiments, the PEgRNA comprises a first direct repeat (the first paring element or the lower stem), wherein a Uracil is changed to a Adenine (such that in the stem region, a U-A base pair is changed to a A-U base pair). In some embodiments, the PEgRNA comprises a first direct repeat wherein the fourth U-A base pair in the stem is changed to a A-U base pair. In some embodiments, the PEgRNA comprises a first direct repeat wherein one or more U-A base pair is changed to a G-C or C-G base pair. For example, in some embodiments, the PEgRNA comprises a first direct repeat comprising a modification to a GUUUU-AAAAC pairing element, wherein one or more of the U-A base pairs is changed to a A-U base pair, a G-C base pair, or a C-G base pair. In some embodiments, the PEgRNA comprises an extended first direct repeat.

In some embodiments, a PEgRNA comprises a gRNA core comprises the sequence

(SEQ ID NO: 3796)
GUUUUAGAGCUAUACGUAGCAAGUUAAAAUAAGGCUAGUCCGUUAUCAA
CUUUACGAAGUGGCACCGAGUCGGUGC
or
(SEQ ID NO: 3797)
GUUUUAGAGCUAUACGUAGCAAGUUAAAAUAAGGCUAGUCCGUUAUCAA
CUUUACGAAGUGGGACCGAGUCGGUCC.

In some embodiments, a PEgRNA comprises a gRNA core comprising the sequence

(SEQ ID NO: 3798)
GUUUUAGAGCUAGCUCAUGAAAAUGAGCUAGCAAGUUAAAAUAAGGCUA
GUCCGUUAUCAACUUGAAAAAGUGGGACCGAGUCGGUCC.

In some embodiments, a PEgRNA comprises a gRNA core comprising the sequence

(SEQ ID NO: 3667)
GUUUGAGAGCUAGAAAUAGCAAGUUUAAAUAAGGCUAGUCCGUUAUCAA
CUUGAAAAAGUGGGACCGAGUCGGUCC.

In some embodiments, a PEgRNA comprises a gRNA core comprising the sequence GUUUAAGAGCUAGAAAUAGCAAGUUUAAAUAAGGCUAGUCCGUUAUCAACUUGAAAAAG UGGCACCGAGUCGGUGC (SEQ ID NO: 3799). In some embodiments, a PEgRNA comprise a gRNA core comprising the sequence GUUUUAGAGCUAUGCUGGAAACAGCAUAGCAAGUUAAAAUAAGGCUAGUCCGUUAUCAA CUUGAAAAAGUGGCACCGAGUCGGUGC (SEQ ID NO: 3800). In some embodiments, a PEgRNA comprise a gRNA core comprising the sequenceGUUUAAGAGCUAUGCUGGAAACAGCAUAGCAAGUUUAAAUAAGGCUAGUCCGUU AUCAACUUGAAAAAGUGGCACCGAGUCGGUGC (SEQ ID NO: 3668).

In some embodiments, the PEgRNA and/or ngRNA comprises a gRNA core that comprises a nucleic acid sequence selected from Table 62 below. In some embodiments, the PEgRNA and/or ngRNA comprises a gRNA core that comprises a nucleic acid sequence that has at least about 50%, 55%, 60%, 65%, 70%, 75%, 80%, 85%, 90%, 95%, 96%, 97%, 98%, or 99% sequence identity to any one of SEQ ID NOs: 3666-3670 or 3796-3800. In some embodiments, the PEgRNA and/or ngRNA comprises a gRNA core that comprises a nucleic acid sequence selected from the group consisting of SEQ ID NOs: 3666-3670 or 3796-3800. In some embodiments, the PEgRNA and/or ngRNA comprises a gRNA core that comprises a nucleic acid sequence of SEQ ID NO: 3666. In some embodiments, the PEgRNA and/or ngRNA comprises a gRNA core that comprises a nucleic acid sequence of SEQ ID NO: 3667. In some embodiments, the PEgRNA and/or ngRNA comprises a gRNA core that comprises a nucleic acid sequence of SEQ ID NO: 3668. In some embodiments, the PEgRNA and/or ngRNA comprises a gRNA core that comprises a nucleic acid sequence of SEQ ID NO: 3669. In some embodiments, the PEgRNA and/or ngRNA comprises a gRNA core that comprises a nucleic acid sequence of SEQ ID NO: 3670.

Table: 62: lists exemplary nucleic acid sequences of gRNA core (gRNA scaffold). The sequences in Table 62 below are annotated with SEQ ID NO as required by ST.26 standard. Although all the sequences provided in Table 62 are RNA sequences, “T” is used instead of a “U” in the sequences for consistency with the ST.26 standard.

TABLE 62
Exemplary nucleic acid sequences of gRNA core (gRNA scaffold)

| SEQ | | |
|---|---|---|
| ID | | |
| NO: | Nucleic acid sequence of gRNA core or scaffold | Remarks |
| 3666 | GTTTTAGAGCTAGAAATAGCAAGTTAAAATAAGGCTAGTCCGTTA | SpCas9 |
| | TCAACTTGAAAAAGTGGCACCGAGTCGGTGC | scaffold |
| 3667 | GTTTGAGAGCTAGAAATAGCAAGTTTAAATAAGGCTAGTCCGTTA | |
| | TCAACTTGAAAAAGTGGGACCGAGTCGGTCC | |
| 3668 | GTTTAAGAGCTATGCTGGAAACAGCATAGCAAGTTTAAATAAGGC | |
| | TAGTCCGTTATCAACTTGAAAAAGTGGCACCGAGTCGGTGC | |
| 3669 | GTTTTAGTACTCTGGAAACAGAATCTACTAAAACAAGGCAAAATG | SaCas9 |
| | CCGTGTTTATCTCGTCAACTTGTTGGCGAGA | gRNA core |
| 3670 | GTTTTAGTACTCTGGAAACAGAATCTACTGAAACAAGACAATATG | SluCas9 |
| | TCGTGTTTATCCCATCAATTTATTGGTGGGA | gRNA core |

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in the disclosure may further comprise nucleotides added to the 5′ of the PEgRNAs. In some embodiments, the PEgRNA further comprises 1, 2, or 3 additional nucleotides added to the 5′ end. The additional nucleotides can be guanine, cytosine, adenine, or uracil. In some embodiments, the additional nucleotide at the 5′ end of the PEgRNA is a guanine or cytosine. In some embodiments, the additional nucleotides can be chemically or biologically modified.

In some embodiments, the PEgRNAs provided in the disclosure may further comprise nucleotides to the 3′ of the PEgRNAs. In some embodiments, the PEgRNA further comprises 1, 2, or 3 additional nucleotides to the 3′ end. The additional nucleotides can be guanine, cytosine, adenine, or uracil. In some embodiments, the additional nucleotides at the 3′ end of the PEgRNA is a polynucleotide comprising at least 1 uracil. In some embodiments, the additional nucleotides can be chemically or biologically modified.

In some embodiments, a PEgRNA or ngRNA is produced by transcription from a template nucleotide, for example, a template plasmid. In some embodiments, a polynucleotide encoding the PEgRNA or ngRNA is appended with one or more additional nucleotides that improves PEgRNA or ngRNA function or expression, e.g., expression from a plasmid that encodes the PEgRNA or ngRNA. In some embodiments, a polynucleotide encoding a PEgRNA or ngRNA is appended with one or more additional nucleotides at the 5′ end or at the 3′ end. In some embodiments, the polynucleotide encoding the PEgRNA or ngRNA is appended with a guanine at the 5′ end, for example, if the first nucleotide at the 5′ end of the spacer is not a guanine. In some embodiments, a polynucleotide encoding the PEgRNA or ngRNA is appended with nucleotide sequence CACC at the 5′ end. In some embodiments, the polynucleotide encoding the PEgRNA or ngRNA is appended with an additional nucleotide adenine at the 3′ end, for example, if the last nucleotide at the 3′ end of the PBS is a Thymine. In some embodiments, the polynucleotide encoding the PEgRNA or ngRNA is appended with additional nucleotide sequence TTTTTT, TTTTTTT, TTTTT, or TTTT at the 3′ end. In some embodiments, the PEgRNA or ngRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises an adenine at the 3′ end, for example, if the last nucleotide at the 3′ end of the PBS is a thymine. In some embodiments, the PEgRNA or ngRNA further comprises nucleotide sequence UUUUUUU, UUUUUU, UUUUU, or UUUU at the 3′ end.

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification can be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification can be within the 3′ most nucleotides of a PEgRNA or ngRNA. In some embodiments, a chemical modification can be within the 3′ most end of a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a chemical modification can be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 3, the gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a ′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification can also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system can be temporally regulated by controlling the timing in which the vectors are delivered. For example, in some embodiments, a polynucleotide encoding the prime editor and a polynucleotide encoding a PEgRNA can be delivered simultaneously. For example, in some embodiments, a polynucleotide encoding the prime editor and a polynucleotide encoding a PEgRNA can be delivered sequentially.

In some embodiments, a polynucleotide encoding a component of a prime editing system can further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Unless otherwise indicated, references to nucleotide positions in human chromosomes are as set forth in human genome assembly consortium Human build 38 (GRCh38), GenBank accession GCF_000001405.38.

Exemplary combinations of PEgRNA components, e.g., spacer, PBS, and editing template/RTT, exemplary full-length PEgRNAs, as well as combinations of PEgRNA and corresponding ngRNA(s) are provided in Tables 1-52. Tables 1-52 each contain three columns. The left column is the sequence number. The middle column provides the sequence of the component, labeled with a SEQ ID NO where required by ST.26 standard. Although all the sequences provided in Tables 1-52 are RNA sequences, “T” is used instead of a “U” in the sequences for consistency with the ST.26 standard. The right column contains a description of the sequence. The RTTs and full-length PEgRNAs in Tables 1-34 are designed to correct a c.67del mutation in the FANCC gene. The RTTs and full length PEgRNAs in Tables 35-52 are designed to correct a c.456+4 A->T mutation. However, the disclosed RTT and full-length PEgRNA are also capable of correcting other mutations in the FANCC gene that are found in the portion of the gene that shares homology or complementarity with the editing template (also referred to as RTT).

The PEgRNAs exemplified in Tables 1-52 comprise: (a) a spacer comprising at its 3′ end a sequence corresponding to a listed PEgRNA spacer sequence; (b) a gRNA core capable of complexing with a Cas9 protein, and (c) an extension arm comprising: (i) an editing template comprising at its 3′ end any RTT sequence from the same table as the PEgRNA spacer, and (ii) a prime binding site (PBS) comprising at its 5′ end any PBS sequence from the same table as the PEgRNA spacer. The PEgRNA spacers in Tables 1-52 are annotated with their PAM sequence(s), enabling the selection of a prime editor comprising an appropriate Cas9 protein. The editing template can encode wildtype FANCC gene sequence (annotated as simply RTT in Tables 1-52). Alternatively, the editing template can encode one or more synonymous mutations relative to the wildtype FANCC gene, e.g., one or more PAM silencing mutations. RTT encoding synonymous PAM silencing mutations are annotated as such in Tables 1-52 in the third column. In some of Tables 1-52, RTT are further annotated with a * followed by a number code. As described below, a PE3b ngRNA spacer annotated with the same * and number code as an RTT has perfect complementarity to the edit strand post-edit by a PEgRNA containing the RTT.

The PEgRNA provided in Tables 1-52 can comprise, from 5′ to 3′, the spacer, the gRNA core, the editing template, and the PBS. The 3′ end of the edit template can be contiguous with the 5′ end of the PBS. The PEgRNA can comprise multiple RNA molecules or can be a single RNA molecule. Any of the PEgRNAs of Tables 1-52 can be used in a Prime Editing system further comprising a nick guide RNA (ngRNA). Such a system may be referred to as a PE3 Prime Editing system. The ngRNA can comprise a spacer comprising at its 3′ end a sequence corresponding to nucleotides 4-20 of any ngRNA spacer listed in the same table as the PEgRNA spacer and a gRNA core capable of complexing with a Cas9 protein. For example, the sequence in the spacer of the ngRNA can comprise nucleotides 4-20, 3-20, 2-20, or 1-20 of the listed spacer. In some embodiments, the spacer of the ngRNA is the complete sequence of an ngRNA spacer listed in the same table as the PEgRNA spacer. The ngRNA spacers in Tables 1-52 are annotated with their PAM sequences, enabling selection of an appropriate Cas9 protein. It can be advantageous to select an ngRNA spacer that has a PAM sequence compatible with the Cas9 protein used in the Prime Editor with the PEgRNA, thus avoiding the need to use two different Cas9 proteins. The ngRNA can comprise multiple RNA molecules (e.g., a crRNA containing the ngRNA spacer and a tracrRNA) or can be a single gRNA molecule. The ngRNA is capable of directing a complexed Cas9 protein to bind the edit strand of the FANCC gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. PE3 ngRNA spacers are simply annotated as is in Tables 1-52. A PE3b spacer annotated with a * followed by a number code has perfect complementarity to the edit strand post-edit with a PEgRNA containing an RTT from the same Table and annotated with the same number code.

Any PEgRNA sequence and/or ngRNA sequence provided in Tables 1-52 may comprise, or further comprise, a 3′ motif at their 3′ end, for example, a series of 1, 2, 3, 4, 5, 6, 7 or more U nucleotides. In some embodiments, the ngRNA comprises 4 U nucleotides at its 3′ end. Without being bound by theory, such 3′ motifs are believed to increase ngRNA stability. The PEgRNA or ngRNA may be chemically synthesized and may alternatively or additionally comprise one or more chemical modifications, such as phosphorothioate (PS) bond(s), 2′-O-methylated (2′-Ome) nucleotides, or a combination thereof. In some embodiments, the PEgRNA and/or the ngRNA comprise 3′ mN*mN*mN*N and 5′mN*mN*mN* modifications, where m indicates that the nucleotide contains a 2′-O-Me modification and a * indicates the presence of a phosphorothioate bond. The PEgRNA sequence or ngRNA sequences may alternatively be adapted for expression from a DNA template, for example, by including a 5′ terminal G if the spacer of the PEgRNA or the ngRNA begins with another nucleotide, by including 6 or 7 U nucleotides at the 3′ end of the ngRNA, or both.

The gRNA core for the PEgRNA and/or the ngRNA can comprises a sequence selected from SEQ ID NOs: 3666-3670 or 3796-3800. In some embodiments, the gRNA core comprises SEQ ID NO: 3666.

Table 3 provides Prime Editing guide RNAs (PEgRNAs) that can be used with any Prime Editor containing a Cas9 protein capable of recognizing an AG or AGG PAM sequence. The PEgRNA of Table 3 can also be used in Prime Editing systems further comprising a nick guide RNA (ngRNA). Such PEgRNAs and Prime Editing systems can be used, for example, to correct a c.67del (322delG) mutation in the FANCC gene. The PEgRNAs exemplified in Table 3 and corresponding Prime Editing systems can also be used to correct any other mutations in the FANCC gene that are found in the portion of the FANCC gene that shares homology or complementarity with the editing template or the RTT.

The PEgRNAs exemplified in Table 3 comprise: (a) a spacer comprising at its 3′ end a sequence corresponding to sequence number 136; (b) a gRNA core capable of complexing with a Cas9 protein, and (c) an extension arm comprising (i) an editing template (or RTT) comprising at its 3′ end a sequence corresponding to sequence number 157 or 158, and (ii) a primer binding site (PBS) comprising at its 5′ end a sequence corresponding to sequence number 142.

The PEgRNA spacers can be, for example, 17 to 22 nucleotides in length, and can comprise the sequence corresponding to any one of sequence numbers 136-141. In some embodiments, the PEgRNA spacer comprises sequence number 139. In some embodiments, the PEgRNA spacer is 20 nucleotides in length and has the sequence of sequence number 139. The PEgRNA spacers in Table 3 are annotated with their corresponding PAM sequence(s), enabling the selection of an appropriate Cas9 protein. The PEgRNA is capable of directing a complexed Prime Editor to bind the non-edit strand of the FANCC gene; thus, a complexed Cas9 nickase portion of the Prime Editor will nick the edit strand. The PEgRNA protospacer may or may not be in close proximity to the position of the c.67 mutation to be corrected by the PEgRNA. The distance between the PEgRNA nick site and the position corresponding to the c.67 mutation (the nick-to-edit distance) is 1 nt for PEgRNAs exemplified in Table 3 that comprise a gRNA core capable of complexing with a SpCas9 nickase.

The editing template, or RTT, can encode a wild type FANCC sequence. For example, the editing template can comprise at its 3′ end the sequence corresponding to sequence number 157, 159, 160, 162, 163, 165, 166, 168, 169, 170, 171, 172, 173, 174, 175, 176, 177, 178, 179, 180, 181, 183, 184, 186, 187, 189, 190, 191, 192, 193, or 194. Alternatively, the editing template can encode a nucleotide edit that corrects the c.67del mutation, and further encode one or more synonymous mutations relative to the wildtype FANCC gene. In some embodiments, the editing template encodes one or more synonymous mutations that are PAM silencing mutations, e.g., a AGG-to-AAG or AG-to-AA PAM silencing mutation, and can comprise the sequence corresponding sequence number 158, 161, 164, 167, 182, 185, or 188. In some embodiments, the editing template is 10 to 40 nucleotides in length. In some embodiments, the editing template is 10 to 32 nucleotides in length. In some embodiments, the editing template is 12 to 16 nucleotides in length and encodes an edit that corrects the c.67del mutation and further encodes an AGG-to-AAG PAM silencing mutation. In some embodiments, the editing template is 10, 12, 14, 16, 30, or 32 nucleotides in length. In some embodiments, the editing template comprises at least 4 contiguous nucleotides of complementarity with the edit strand wherein the at least 4 nucleotides contiguous are located upstream of the 5′ most edit in the editing template.

The PBS can be, for example, 5 to 19 nucleotides in length and can comprise the sequence corresponding to any one of sequence numbers 142-156. In some embodiments, the PBS is 8 to 14 nucleotides in length. In some embodiments, the PBS is 14 nucleotides in length. In some embodiments, the PBS of the PEgRNA is a PBS sequence provided in sequence number 145, 146, 147, 148, 149, or 150.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Table 3 can comprise a sequence corresponding to any one of sequence numbers 219-322 and 3592-3603. Any PEgRNA exemplified in Table 3 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Table 3 may be adapted for expression from a nucleic acid template with a U6 promoter, for example, by including a 5′ terminal G if the spacer of the PEgRNA begins with another nucleotide. The modifications and adaptations included in the selection of full length PEgRNAs included in Table 3 are annotated in the third column (“Description”) of Table 3.

The PEgRNAs exemplified in Table 3 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Any of the PEgRNAs of Table 3 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in Table 3 and a gRNA core capable of complexing with a Cas9 protein. For example, the sequence in the spacer of the ngRNA can comprise nucleotides 4-20, 3-20, 2-20, or 1-20 of any one of sequence numbers 4 and 195-218. In some embodiments, the spacer of the ngRNA is a ngRNA spacer listed in Table 3. The ngRNA spacers in Table 3 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. In some embodiments, the distance between the PEgRNA nick site and the ngRNA nick site (the nick-to-nick distance) is about 3 to 100 nt. In some embodiments, the distance between the PEgRNA nick site and the ngRNA nick site (the nick-to-nick distance) is about 21 to 96 nt. In some embodiments, the distance between the PEgRNA nick site and the ngRNA nick site (the nick-to-nick distance) is at least 22 nt. In some embodiments, the distance between the PEgRNA nick site and the ngRNA nick site (the nick-to-nick distance) is about 22 to 97 nt. In some embodiments, the distance between the PEgRNA nick site and the ngRNA nick site (the nick-to-nick distance) is about 77 to 97 nt. In some embodiments, the distance between the PEgRNA nick site and the ngRNA nick site (the nick-to-nick distance) is about 22, 34, 35, 46, 68, 77, or 97 nt. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. Exemplary ngRNA provided in Table 3 can comprise a sequence corresponding to any one of sequence numbers 323-343 and 3604-3610.

Any ngRNA exemplified in Table 3 can comprise, or further comprise, a 3′ motif at the 3′ end. In some embodiments, a 3′ motif is capable of forming a tertiary structure on its own, such as a hairpin. The 3′ motif can be connected to the 3′ end of the ngRNA via a linker sequence. The ngRNA may also comprise adaptations at the 3′ end or 5′ end. For example, the ngRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Table 3 may alternatively be adapted for expression from a nucleic acid template with a U6 promoter, for example, by including a 5′ terminal G if the spacer of the ngRNA begins with another nucleotide. The modifications and adaptations included in the selection of full length ngRNAs included in Table 3 are annotated in the third column (“Description”) of Table 3.

The ngRNAs exemplified in Table 3 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Table 15 provides Prime Editing guide RNAs (PEgRNAs) that can be used with any Prime Editor containing a Cas9 protein capable of recognizing a TG or TGG PAM sequence. The PEgRNA of Table 15 can also be used in Prime Editing systems further comprising a nick guide RNA (ngRNA). Such PEgRNAs and Prime Editing systems can be used, for example, to correct a c.67del (322delG) mutation in the FANCC gene. The PEgRNAs exemplified in Table 15 and corresponding Prime Editing systems can also be used to correct any other mutations in the FANCC gene that are found in the portion of the FANCC gene that shares homology or complementarity with the editing template or the RTT.

The PEgRNAs exemplified in Table 15 comprise: (a) a spacer comprising at its 3′ end a sequence corresponding to sequence number 915; (b) a gRNA core capable of complexing with a Cas9 protein, and (c) an extension arm comprising (i) an editing template (or RTT) comprising at its 3′ end a sequence corresponding to sequence number 935, and (ii) a primer binding site (PBS) comprising at its 5′ end a sequence corresponding to sequence number 920.

The PEgRNA spacers can be, for example, 17 to 22 nucleotides in length, and can comprise the sequence corresponding to any one of sequence numbers 915-917, 35, 918, and 919. In some embodiments, the PEgRNA spacer comprises sequence number 35. In some embodiments, the PEgRNA spacer is 20 nucleotides in length and has the sequence of sequence number 35. The PEgRNA spacers in Table 15 are annotated with their corresponding PAM sequence(s), enabling the selection of an appropriate Cas9 protein. The PEgRNA is capable of directing a complexed Prime Editor to bind the non-edit strand of the FANCC gene; thus, a complexed Cas9 nickase portion of the Prime Editor will nick the edit strand. The PEgRNA protospacer may or may not be in close proximity to the position of the c.67 mutation to be corrected by the PEgRNA. The distance between the PEgRNA nick site and the position corresponding to the c.67 mutation (the nick-to-edit distance) is 7 nt for PEgRNAs exemplified in Table 15 that comprise a gRNA core capable of complexing with a SpCas9 nickase.

The editing template, or RTT, can encode a wild type FANCC sequence. For example, the editing template can comprise at its 3′ end the sequence corresponding to sequence number 935, 936, 937, 938, 939, 940, 941, 942, 943, 944, 384, 945, 946, 947, 948, 949, 950, 951, 952, 953, 954, 955, 956, 957, 958, 959, 960, 961, 962, 963, or 964. Alternatively, the editing template can encode a nucleotide edit that corrects the c.67del mutation, and further encode one or more synonymous mutations relative to the wildtype FANCC gene. In some embodiments, the editing template encodes one or more synonymous mutations that are PAM silencing mutations. In some embodiments, the editing template is 10 to 40 nucleotides in length. In some embodiments, the editing template is at least 11 nucleotides in length. In some embodiments, the editing template is 11 to 20 nucleotides in length. In some embodiments, the editing template comprises at least 4 contiguous nucleotides of complementarity with the edit strand wherein the at least 4 nucleotides contiguous are located upstream of the 5′ most edit in the editing template.

The PBS can be, for example, 5 to 19 nucleotides in length and can comprise the sequence corresponding to any one of sequence numbers 920-934. In some embodiments, the PBS is 8 to 14 nucleotides in length. In some embodiments, the PBS is 8 to 12 nucleotides in length. In some embodiments, the PBS is 8, 10, 12, or 14 nucleotides in length. In some embodiments, the PBS of the PEgRNA is a PBS sequence provided in sequence number 923, 925, 927, or 929.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Table 15 can comprise a sequence corresponding to any one of sequence numbers 965-1024 and 3611-3630. Any PEgRNA exemplified in Table 15 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Table 15 may be adapted for expression from a nucleic acid template with a U6 promoter, for example, by including a 5′ terminal G if the spacer of the PEgRNA begins with another nucleotide. The modifications and adaptations included in the selection of full length PEgRNAs included in Table 15 are annotated in the third column (“Description”) of Table 15.

The PEgRNAs exemplified in Table 15 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Any of the PEgRNAs of Table 15 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in Table 15 and a gRNA core capable of complexing with a Cas9 protein. For example, the sequence in the spacer of the ngRNA can comprise nucleotides 4-20, 3-20, 2-20, or 1-20 of any one of sequence number 4,195,196, 197, 199, 200, 201, 202, 203, 204, 205, 206, 208, 209, 210, 211, 212, 213, 384, 214, 215, 216, 217, or 218. In some embodiments, the spacer of the ngRNA is a ngRNA spacer listed in Table 15. The ngRNA spacers in Table 15 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. In some embodiments, the distance between the PEgRNA nick site and the ngRNA nick site (the nick-to-nick distance) is about 3 to 100 nt. In some embodiments, the distance between the PEgRNA nick site and the ngRNA nick site (the nick-to-nick distance) is about 28 to 103 nt. In some embodiments, the distance between the PEgRNA nick site and the ngRNA nick site (the nick-to-nick distance) is at least 28 nt. In some embodiments, the distance between the PEgRNA nick site and the ngRNA nick site (the nick-to-nick distance) is about 83 to 103 nt. In some embodiments, the distance between the PEgRNA nick site and the ngRNA nick site (the nick-to-nick distance) is about 28, 40, 41, 52, 74, 83, or 103 nt. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. Exemplary ngRNA provided in Table 15 can comprise a sequence corresponding to any one of sequence numbers 323-343 and 3604-3610.

Any ngRNA exemplified in Table 15 can comprise, or further comprise, a 3′ motif at the 3′ end. In some embodiments, a 3′ motif is capable of forming a tertiary structure on its own, such as a hairpin. The 3′ motif can be connected to the 3′ end of the ngRNA via a linker sequence. The ngRNA may also comprise adaptations at the 3′ end or 5′ end. For example, the ngRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Table 15 may be adapted for expression from a nucleic acid template with a U6 promoter, for example, by including a 5′ terminal G if the spacer of the ngRNA begins with another nucleotide. The modifications and adaptations included in the selection of full length ngRNAs included in Table 15 are annotated in the third column (“Description”) of Table 15.

The ngRNA exemplified in Table 15 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Table 18 provides Prime Editing guide RNAs (PEgRNAs) that can be used with any Prime Editor containing a Cas9 protein capable of recognizing a TG or TGG PAM sequence. The PEgRNA of Table 18 can also be used in Prime Editing systems further comprising a nick guide RNA (ngRNA). Such PEgRNAs and Prime Editing systems can be used, for example, to correct a c.67del (322delG) mutation in the FANCC gene. The PEgRNAs exemplified in Table 18 and corresponding Prime Editing systems can also be used to correct any other mutations in the FANCC gene that are found in the portion of the FANCC gene that shares homology or complementarity with the editing template or the RTT.

The PEgRNAs exemplified in Table 18 comprise: (a) a spacer comprising at its 3′ end a sequence corresponding to sequence number 1230; (b) a gRNA core capable of complexing with a Cas9 protein, and (c) an extension arm comprising (i) an editing template (or RTT) comprising at its 3′ end a sequence corresponding to sequence number 1250, 1251, 1252, or 1253, and (ii) a primer binding site (PBS) comprising at its 5′ end a sequence corresponding to sequence number 1235.

The PEgRNA spacers can be, for example, 17 to 22 nucleotides in length, and can comprise the sequence corresponding to any one of sequence numbers 1230-1232, 212, 1233, and 1234. In some embodiments, the PEgRNA spacer comprises sequence number 212. In some embodiments, the PEgRNA spacer is 20 nucleotides in length and has the sequence of sequence number 212. The PEgRNA spacers in Table 18 are annotated with their corresponding PAM sequence(s), enabling the selection of an appropriate Cas9 protein. The PEgRNA is capable of directing a complexed Prime Editor to bind the non-edit strand of the FANCC gene; thus, a complexed Cas9 nickase portion of the Prime Editor will nick the edit strand. The PEgRNA protospacer may or may not be in close proximity to the position of the c.67 mutation to be corrected by the PEgRNA. The distance between the PEgRNA nick site and the position corresponding to the c.67 mutation (the nick-to-edit distance) is 16 nt for PEgRNAs exemplified in Table 18 that comprise a gRNA core capable of complexing with a SpCas9 nickase.

The editing template, or RTT, can encode a wild type FANCC sequence. For example, the editing template can comprise at its 3′ end the sequence corresponding to sequence number 1253, 1259, 1265, 1271, 28, 1282, 1288, 1294, 1300, 1306, 1312, 1318, 1324, 1330, 1336, 1342, 1348, 1354, 1360, 1366, 1372, 1378, 1384, 1390, or 1396. Alternatively, the editing template can encode a nucleotide edit that corrects the c.67del mutation, and further encode one or more synonymous mutations relative to the wildtype FANCC gene. In some embodiments, the editing template encodes one or more synonymous mutations that are PAM silencing mutations. For example, in some embodiments, the editing template encodes one or more synonymous mutations that are PAM silencing mutations and can comprise at its 3′ end the sequence corresponding to sequence number 1254, 1255, 1256, 1257, 1258, 1260, 1261, 1262, 1263, 1264, 1266, 1267, 1268, 1269, 1270, 1272, 1273, 1274, 1275, 1276, 1277, 1278, 1279, 1280, 1281, 1283, 1284, 1285, 1286, 1287, 1289, 1290, 1291, 1292, 1293, 1295, 1296, 1297, 1298, 1299, 1301, 1302, 1303, 1304, 1305, 1307, 1308, 1309, 1310, 1311, 1313, 1314, 1315, 1316, 1317, 1319, 1320, 1321, 1322, 1323, 1325, 1326, 1327, 1328, 1329, 1331, 1332, 1333, 1334, 1335, 1337, 1338, 1339, 1340, 1341, 1343, 1344, 1345, 1346, 1347, 1349, 1350, 1351, 1352, 1353, 1355, 1356, 1357, 1358, 1359, 1361, 1362, 1363, 1364, 1365, 1367, 1368, 1369, 1370, 1371, 1373, 1374, 1375, 1376, 1377, 1379, 1380, 1381, 1382, 1383, 1385, 1386, 1387, 1388, 1389, 1391, 1392, 1393, 1394, 1395, 1397, or 1398. The PAM silencing mutations encoded by exemplary RTTs are annotated in Table 18, third column (“Description”). In some embodiments, the editing template is 10 to 40 nucleotides in length. In some embodiments, the editing template is 20 to 29 nucleotides in length. In some embodiments, the editing template is at least 25 nucleotides in length. In some embodiments, the editing template is 25 nucleotides in length.

The PBS can be, for example, 5 to 19 nucleotides in length and can comprise the sequence corresponding to any one of sequence numbers 1235-1249. In some embodiments, the PBS is 8 to 14 nucleotides in length. In some embodiments, the PBS is 8, 10, 12, or 14 nucleotides in length. In some embodiments, the PBS of the PEgRNA is a PBS sequence provided in sequence number 1238, 1240, 1242, or 1244. In some embodiments, the editing template comprises at least 4 contiguous nucleotides of complementarity with the edit strand wherein the at least 4 nucleotides contiguous are located upstream of the 5′ most edit in the editing template.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Table 18 can comprise a sequence corresponding to any one of sequence numbers 1403-1462 and 3631-3650. Any PEgRNA exemplified in Table 18 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Table 18 may be adapted for expression from a nucleic acid template with a U6 promoter, for example, by including a 5′ terminal G if the spacer of the PEgRNA begins with another nucleotide. The modifications and adaptations included in the selection of full length PEgRNAs included in Table 18 are annotated in the third column (“Description”) of Table 18.

The PEgRNA exemplified in Table 18 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. Any of the PEgRNAs of Table 18 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in Table 18 and a gRNA core capable of complexing with a Cas9 protein. For example, the sequence in the spacer of the ngRNA can comprise nucleotides 4-20, 3-20, 2-20, or 1-20 of any one of sequence number 24, 25, 26, 27, 1274, 1275, 1276, 28, 1277, 1278, 1108, 29, 1110, 30, 31, 32, 33, 1111, 34, 35, 37, 38, 39, 41, 1113, 1399, 1400, 43, 1401, 1402, 44, 1114, 45, 46, 47, or 48. In some embodiments, the spacer of the ngRNA is a ngRNA spacer listed in Table 18. The ngRNA spacers in Table 18 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. In some embodiments, the distance between the PEgRNA nick site and the ngRNA nick site (the nick-to-nick distance) is about 3 to 100 nt. In some embodiments, the distance between the PEgRNA nick site and the ngRNA nick site (the nick-to-nick distance) is about 19 to 58 nt. In some embodiments, the distance between the PEgRNA nick site and the ngRNA nick site (the nick-to-nick distance) is about 19, 31, 37, or 58 nt. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 18 annotated with a “*” and the same number code as an RTT in Table 18 has perfect complementarity to the edit strand post-edit by a PEgRNA containing the RTT. Exemplary ngRNA provided in Table 18 can comprise a sequence corresponding to any one of sequence numbers 49-60.

Any ngRNA exemplified in Table 18 can comprise, or further comprise, a 3′ motif at the 3′ end. In some embodiments, a 3′ motif is capable of forming a tertiary structure on its own, such as a hairpin. The 3′ motif can be connected to the 3′ end of the ngRNA via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Table 18 may be adapted for expression from a nucleic acid template with a U6 promoter, for example, by including a 5′ terminal G if the spacer of the ngRNA begins with another nucleotide. The modifications and adaptations included in the selection of full length ngRNAs included in Table 18 are annotated in the third column (“Description”) of Table 18.

The ngRNA exemplified in Table 18 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Table 19 provides Prime Editing guide RNAs (PEgRNAs) that can be used with any Prime Editor containing a Cas9 protein capable of recognizing a GG or GGG PAM sequence. The PEgRNA of Table 19 can also be used in Prime Editing systems further comprising a nick guide RNA (ngRNA). Such PEgRNAs and Prime Editing systems can be used, for example, to correct a c.67del (322delG) mutation in the FANCC gene. The PEgRNAs exemplified in Table 19 and corresponding Prime Editing systems can also be used to correct any other mutations in the FANCC gene that are found in the portion of the FANCC gene that shares homology or complementarity with the editing template or the RTT.

The PEgRNAs exemplified in Table 19 comprise: (a) a spacer comprising at its 3′ end a sequence corresponding to sequence number 1463; (b) a gRNA core capable of complexing with a Cas9 protein, and (c) an extension arm comprising (i) an editing template (or RTT) comprising at its 3′ end a sequence corresponding to sequence number 1483, 1484, 1485, or 1486, and (ii) a primer binding site (PBS) comprising at its 5′ end a sequence corresponding to sequence number 1468.

The PEgRNA spacers can be, for example, 17 to 22 nucleotides in length, and can comprise the sequence corresponding to any one of sequence numbers 1463-1465, 213, 1466, and 1467. In some embodiments, the PEgRNA spacer comprises sequence number 212. In some embodiments, the PEgRNA spacer is 20 nucleotides in length and has the sequence of sequence number 213. The PEgRNA spacers in Table 19 are annotated with their corresponding PAM sequence(s), enabling the selection of an appropriate Cas9 protein. The PEgRNA is capable of directing a complexed Prime Editor to bind the non-edit strand of the FANCC gene; thus, a complexed Cas9 nickase portion of the Prime Editor will nick the edit strand. The PEgRNA protospacer may or may not be in close proximity to the position of the c.67 mutation to be corrected by the PEgRNA. The distance between the PEgRNA nick site and the position corresponding to the c.67 mutation (the nick-to-edit distance) is 28 nt for PEgRNAs exemplified in Table 19 that comprise a gRNA core capable of complexing with a SpCas9 nickase.

The editing template, or RTT, can encode a wild type FANCC sequence. For example, the editing template can comprise at its 3′ end the sequence corresponding to sequence number 1484, 1488, 1492, 1496, 1500, 1504, 1508, 1512, 1516, 1520, 1524, 1528, or 1532. Alternatively, the editing template can encode a nucleotide edit that corrects the c.67del mutation, and further encode one or more synonymous mutations relative to the wildtype FANCC gene. In some embodiments, the editing template encodes one or more synonymous mutations that are PAM silencing mutations. For example, in some embodiments, the editing template encodes one or more synonymous mutations that are PAM silencing mutations and can comprise at its 3′ end the sequence corresponding to sequence number 1483, 1485, 1486, 1487, 1489, 1490, 1491, 1493, 1494, 1495, 1497, 1498, 1499, 1501, 1502, 1503, 1505, 1506, 1507, 1509, 1510, 1511, 1513, 1514, 1515, 1517, 1518, 1519, 1521, 1522, 1523, 1525, 1526, 1527, 1529, 1530, 1531, 1533, or 1534. The PAM silencing mutations encoded by exemplary RTTs are annotated in Table 19, third column (“Description”). In some embodiments, the editing template is 10 to 40 nucleotides in length. In some embodiments, the editing template is 32 to 39 nucleotides in length. In some embodiments, the editing template is 39 nucleotides in length. In some embodiments, the editing template comprises at least 4 contiguous nucleotides of complementarity with the edit strand wherein the at least 4 nucleotides contiguous are located upstream of the 5′ most edit in the editing template.

The PBS can be, for example, 5 to 19 nucleotides in length and can comprise the sequence corresponding to any one of sequence numbers 1468-1482. In some embodiments, the PBS is 8 to 14 nucleotides in length. In some embodiments, the PBS is 8, 10, 12, or 14 nucleotides in length. In some embodiments, the PBS of the PEgRNA is a PBS sequence provided in sequence number 1471, 1473, 1475, or 1477.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Table 19 can comprise a sequence corresponding to any one of sequence numbers 1541-1588 and 3651-3665. Any PEgRNA exemplified in Table 19 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Table 19 may be adapted for expression from a nucleic acid template with a U6 promoter, for example, by including a 5′ terminal G if the spacer of the PEgRNA begins with another nucleotide. The modifications and adaptations included in the selection of full length PEgRNAs included in Table 19 are annotated in the third column (“Description”) of Table 19.

The PEgRNAs exemplified in Table 19 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Any of the PEgRNAs of Table 19 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in Table 19 and a gRNA core capable of complexing with a Cas9 protein. For example, the sequence in the spacer of the ngRNA can comprise nucleotides 4-20, 3-20, 2-20, or 1-20 of any one of sequence number 24, 25, 26, 27, 28, 29, 30, 31, 32, 33, 34, 35, 1535, 36, 1536, 1537, 37, 38, 39, 1538, 40, 1539, 1540, 41, 43, 44, 45, 46, 47, or 48. In some embodiments, the spacer of the ngRNA is a ngRNA spacer listed in Table 19. The ngRNA spacers in Table 19 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. In some embodiments, the distance between the PEgRNA nick site and the ngRNA nick site (the nick-to-nick distance) is about 3 to 100 nt. In some embodiments, the distance between the PEgRNA nick site and the ngRNA nick site (the nick-to-nick distance) is about 31 to 70 nt. In some embodiments, the distance between the PEgRNA nick site and the ngRNA nick site (the nick-to-nick distance) is about 31, 43, 49, or 70 nt. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. A PE3 or PE3b ngRNA spacer in Table 19 annotated with a “*” and the same number code as an RTT in Table 19 has perfect complementarity to the edit strand post-edit by a PEgRNA containing the RTT. Exemplary ngRNA provided in Table 19 can comprise a sequence corresponding to any one of sequence numbers 49-60.

Any ngRNA exemplified in Table 19 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the ngRNA via a linker sequence. The ngRNA may also comprise adaptations at the 3′ end or 5′ end. For example, the ngRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Table 19 may be adapted for expression from a nucleic acid template with a U6 promoter, for example, by including a 5′ terminal G if the spacer of the ngRNA begins with another nucleotide. The modifications and adaptations included in the selection of full length ngRNAs included in Table 19 are annotated in the third column (“Description”) of Table 19.

The ngRNAs exemplified in Table 19 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Additional exemplary PEgRNAs and ngRNAs for correction of the c.67del mutation are provided in Tables 1, 2, 4-14, 16, 17, and 20-34. Tables 1, 2, 4-14, 16, 17, and 20-34 each contain three columns: the first column is the sequence number, the second column is the actual sequence, and the third column contains a description of the sequence.

The PEgRNAs exemplified in Tables 1, 2, 4-14, 16, 17, and 20-34 comprise: (a) a spacer comprising at its 3′ end a sequence corresponding to a listed PEgRNA spacer sequence; (b) a gRNA core capable of complexing with a Cas9 protein, and (c) an extension arm comprising: (i) an editing template comprising at its 3′ end an RTT sequence from the same table as the PEgRNA spacer, and (ii) a prime binding site (PBS) comprising at its 5′ end a PBS sequence from the same table as the PEgRNA spacer.

The PEgRNA spacer can be, for example, 16-22 nucleotides in length. The PEgRNA spacers in Tables 1, 2, 4-14, 16, 17, and 20-34 are annotated with their PAM sequence(s), enabling the selection of an appropriate Cas9 protein.

The editing template, or RTT, can encode a wild type FANCC sequence. Alternatively, the editing template can encode a nucleotide edit that corrects the c.67del mutation, and further encode one or more synonymous mutations relative to the wildtype FANCC gene. In some embodiments, the editing template encodes one or more synonymous mutations that are PAM silencing mutations. The PAM silencing mutations encoded by exemplary RTTs are annotated in each of Tables 1, 2, 4-14, 16, 17, and 20-34, third column (“Description”). In some embodiments, the editing template is 10 to 40 nucleotides in length. In some embodiments, the editing template comprises at least 4 contiguous nucleotides of complementarity with the edit strand wherein the at least 4 nucleotides contiguous are located upstream of the 5′ most edit in the editing template.

The PBS can be, for example, 5 to 19 nucleotides in length. In some cases, a PBS length of no more than 3 nucleotides less than the PEgRNA spacer length is chosen. In some embodiments, a PBS length between 8 to 14 nucleotides is chosen.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Tables 1, 2, 4-14, 16, 17, and 20-34 can comprise a sequence corresponding to any one of full length PEgRNA sequences in each table. Any PEgRNA exemplified in Tables 1, 2, 4-14, 16, 17, and 20-34 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Tables 1, 2, 4-14, 16, 17, and 20-34 may be adapted for expression from a nucleic acid template with a U6 promoter, for example, by including a 5′ terminal G if the spacer of the PEgRNA begins with another nucleotide. The modifications and end adaptations included in the selection of full length PEgRNAs included in Tables 1, 2, 4-14, 16, 17, and 20-34 are annotated in the third column (“Description”) of the Tables.

The PEgRNAs exemplified in Tables 1, 2, 4-14, 16, 17, and 20-34 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Any of the PEgRNAs of Tables 1, 2, 4-14, 16, 17, and 20-34 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in the same Table as the PEgRNA and a gRNA core capable of complexing with a Cas9 protein. In some embodiments, the spacer of the ngRNA is a ngRNA spacer listed in the same Table as the PEgRNA. The ngRNA spacers in each of Tables 1, 2, 4-14, 16, 17, and 20-34 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. In some embodiments, the distance between the PEgRNA nick site and the ngRNA nick site (the nick-to-nick distance) is about 3 to 100 nt. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. A PE3 or PE3b ngRNA spacer in Tables 1, 2, 4-14, 16, 17, and 20-34 annotated with a “*” and the same number code as an RTT in the same Table has perfect complementarity to the edit strand post-edit by a PEgRNA containing the RTT. Exemplary ngRNA provided in Tables 1, 2, 4-14, 16, 17, and 20-34 can comprise a sequence corresponding to any one of the full length ngRNAs provided in the Tables.

Any ngRNA exemplified in Tables 1, 2, 4-14, 16, 17, and 20-34 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the ngRNA via a linker sequence. The ngRNA may also comprise adaptations at the 3′ end or 5′ end. For example, the ngRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Tables 1, 2, 4-14, 16, 17, and 20-34 may be adapted for expression from a nucleic acid template with a U6 promoter, for example, by including a 5′ terminal G if the spacer of the ngRNA begins with another nucleotide. The modifications and end adaptations included in the selection of full length ngRNAs included in Tables 1, 2, 4-14, 16, 17, and 20-34 are annotated in the third column (“Description”) of the Tables.

The ngRNA exemplified in Tables 1, 2, 4-14, 16, 17, and 20-34 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-0-Me modification and a * indicates the presence of a phosphorothioate bond.

Table 45 provides Prime Editing guide RNAs (PEgRNAs) that can be used with any Prime Editor containing a Cas9 protein capable of recognizing a TG or TGG PAM sequence. The PEgRNA of Table 45 can also be used in Prime Editing systems further comprising a nick guide RNA (ngRNA). Such PEgRNAs and Prime Editing systems can be used, for example, to correct a c.456+4A->T (IVS4+4 A->T) mutation in the FANCC gene. The PEgRNAs exemplified in Table 45 and corresponding Prime Editing systems can also be used to correct any other mutations in the FANCC gene that are found in the portion of the FANCC gene that shares homology or complementarity with the editing template or the RTT.

The PEgRNAs exemplified in Table 45 comprise: (a) a spacer comprising at its 3′ end a sequence corresponding to sequence number 3086; (b) a gRNA core capable of complexing with a Cas9 protein, and (c) an extension arm comprising (i) an editing template (or RTT) comprising at its 3′ end a sequence corresponding to sequence number 3106, and (ii) a primer binding site (PBS) comprising at its 5′ end a sequence corresponding to sequence number 3091.

The PEgRNA spacers can be, for example, 17 to 22 nucleotides in length, and can comprise the sequence corresponding to any one of sequence numbers 3086-3088, 2619, 3089, and 3090. In some embodiments, the PEgRNA spacer comprises sequence number 2619. In some embodiments, the PEgRNA spacer is 20 nucleotides in length and has the sequence of sequence number 2619. The PEgRNA spacers in Table 45 are annotated with their corresponding PAM sequence(s), enabling the selection of an appropriate Cas9 protein. The PEgRNA is capable of directing a complexed Prime Editor to bind the non-edit strand of the FANCC gene; thus, a complexed Cas9 nickase portion of the Prime Editor will nick the edit strand. The PEgRNA protospacer may or may not be in close proximity to the position of the c.456+4 A->T mutation to be corrected by the PEgRNA. The distance between the PEgRNA nick site and the position corresponding to the c.456+4 A->T mutation (the nick-to-edit distance) is 23 nt for PEgRNAs exemplified in Table 45 that comprise a gRNA core capable of complexing with a SpCas9 nickase.

The editing template, or RTT, can encode a wild type FANCC sequence. For example, the editing template can comprise at its 3′ end the sequence corresponding to any one of sequence numbers 3106-3123. Alternatively, the editing template can encode a nucleotide edit that corrects the c.456+4 A->T mutation, and further encode one or more synonymous mutations relative to the wildtype FANCC gene. In some embodiments, the editing template is 10 to 40 nucleotides in length. In some embodiments, the editing template is 27 to 33 nucleotides in length. In some embodiments, the editing template comprises at least 4 contiguous nucleotides of complementarity with the edit strand wherein the at least 4 nucleotides contiguous are located upstream of the 5′ most edit in the editing template.

The PBS can be, for example, 5 to 19 nucleotides in length and can comprise the sequence corresponding to any one of sequence numbers 3091-3105. In some embodiments, the PBS is 8 to 14 nucleotides in length. In some embodiments, the PBS of the PEgRNA is a PBS sequence provided in sequence number 3094, 3096, 3098, or 3100.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Table 45 can comprise a sequence corresponding to sequence number 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, or 3155.

Any PEgRNA exemplified in Table 45 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Table 45 may alternatively be adapted for expression from a nucleic acid template with a U6 promoter, for example, by including a 5′ terminal G if the spacer of the PEgRNA begins with another nucleotide. The end adaptations included in the selection of full length PEgRNAs included in Table 45 are annotated in the third column (“Description”) of Table 45.

The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Any of the PEgRNAs of Table 45 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in Table 45 and a gRNA core capable of complexing with a Cas9 protein. For example, the sequence in the spacer of the ngRNA can comprise nucleotides 4-20, 3-20, 2-20, or 1-20 of sequence number 2885, 2559, 2886, 2573, 2887, 2888, 2561, 2889, 2890, 2891, 2892, 2893, 2569, 2894, 2895, or 2896. In some embodiments, the spacer of the ngRNA is a ngRNA spacer listed in Table 45. The ngRNA spacers in Table 45 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. In some embodiments, the distance between the PEgRNA nick site and the ngRNA nick site (the nick-to-nick distance) is 131 to 134 nt. In some embodiments, the distance between the PEgRNA nick site and the ngRNA nick site (the nick-to-nick distance) is up to 134 nt. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. Exemplary ngRNA provided in Table 45 can comprise a sequence corresponding to any one of sequence numbers 2897-2900.

Any ngRNA exemplified in Table 45 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the ngRNA via a linker sequence. The ngRNA may also comprise adaptations at the 3′ end or 5′ end. For example, the ngRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Table 45 may be adapted for expression from a nucleic acid template with a U6 promoter, for example, by including a 5′ terminal G if the spacer of the ngRNA begins with another nucleotide. The end adaptations included in the selection of full length ngRNAs included in Table 45 are annotated in the third column (“Description”) of Table 45.

The ngRNAs exemplified in Table 45 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Additional exemplary PEgRNAs and ngRNAs for correction of the c.456+4A->T (IVS4+4 A->T) mutation are provided in Tables 35-44 and 46-52. Tables 35-44 and 46-524 each contain three columns: the first column is the sequence number, the second column is the actual sequence, and the third column contains a description of the sequence.

The PEgRNAs exemplified in Tables 35-44 and 46-52 comprise: (a) a spacer comprising at its 3′ end a sequence corresponding to a listed PEgRNA spacer sequence; (b) a gRNA core capable of complexing with a Cas9 protein, and (c) an extension arm comprising: (i) an editing template comprising at its 3′ end an RTT sequence from the same table as the PEgRNA spacer, and (ii) a prime binding site (PBS) comprising at its 5′ end a PBS sequence from the same table as the PEgRNA spacer.

The PEgRNA spacer can be, for example, 16-22 nucleotides in length. The PEgRNA spacers in Tables 35-44 and 46-52 are annotated with their PAM sequence(s), enabling the selection of an appropriate Cas9 protein.

The editing template, or RTT, can encode a wild type FANCC sequence. Alternatively, the editing template can encode a nucleotide edit that corrects the c c.456+4A->T (IVS4+4 A->T) mutation, and further encode one or more synonymous mutations relative to the wildtype FANCC gene. In some embodiments, the editing template encodes one or more synonymous mutations that are PAM silencing mutations. The PAM silencing mutations encoded by exemplary RTTs are annotated in each of Tables 35-44 and 46-52, third column (“Description”). In some embodiments, the editing template is 10 to 40 nucleotides in length. In some embodiments, the editing template comprises at least 4 contiguous nucleotides of complementarity with the edit strand wherein the at least 4 nucleotides contiguous are located upstream of the 5′ most edit in the editing template.

The PBS can be, for example, 5 to 19 nucleotides in length. In some cases, a PBS length of no more than 3 nucleotides less than the PEgRNA spacer length is chosen. In some embodiments, a PBS length between 8 to 14 nucleotides is chosen.

The PEgRNA can comprise multiple RNA molecules or can be a single RNA molecule. In some embodiments, the PEgRNA comprises, from 5′ to 3′, the spacer, the gRNA core, the editing template, and the PBS. The 3′ end of the editing template can be contiguous with the 5′ end of the PBS. Exemplary PEgRNAs provided in Tables 35-44 and 46-52 can comprise a sequence corresponding to any one of full length PEgRNA sequences in each table. Any PEgRNA exemplified in Tables 35-44 and 46-52 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the PBS via a linker sequence. The PEgRNA may also comprise adaptations at the 3′ end or 5′ end. For example, the PEgRNA may comprise a series of 1, 2, 3, 4, 5, 6, 7 or more additional Uracil nucleotides at the 3′ end. In some embodiments, the PEgRNA comprises 4 additional Uracil nucleotides at its 3′ end. Without being bound by theory, the 3 motifs or additional Uracil nucleotides can increase PEgRNA stability. PEgRNA sequences exemplified in Tables 35-44 and 46-52 may be adapted for expression from a nucleic acid template with a U6 promoter, for example, by including a 5′ terminal G if the spacer of the PEgRNA begins with another nucleotide. The modifications and end adaptations included in the selection of full length PEgRNAs included in Tables 35-44 and 46-52 are annotated in the third column (“Description”) of the Tables.

The PEgRNAs exemplified in Tables 35-44 and 46-52 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

Any of the PEgRNAs of Tables 35-44 and 46-52 can be used in a Prime Editing system that further comprises a nick guide RNA (ngRNA). Such ngRNA can comprise a spacer comprising at its 3′ end a sequence corresponding to nucleotides 4-20 of any ngRNA spacer listed in the same Table as the PEgRNA and a gRNA core capable of complexing with a Cas9 protein. In some embodiments, the spacer of the ngRNA is a ngRNA spacer listed in the same Table as the PEgRNA. The ngRNA spacers in each of Tables 35-44 and 46-52 are annotated with their corresponding PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the FANCC gene; thus, a complexed Cas9 nickase containing a nuclease inactivating mutation in the HNH domain will nick the non-edit strand. The ngRNA protospacer may or may not be in close proximity to the PEgRNA protospacer. The ngRNA can be a PE3 ngRNA or a PE3b ngRNA: A PE3 ngRNA spacer has perfect complementarity to the edit strand both pre- and post-edit; a PE3b ngRNA spacer has perfect complementarity to the edit strand post-edit. A PE3 or PE3b ngRNA spacer in Tables 35-44 and 46-52 annotated with a “*” and the same number code as an RTT in the same Table has perfect complementarity to the edit strand post-edit by a PEgRNA containing the RTT. Exemplary ngRNA provided in Tables 35-44 and 46-52 can comprise a sequence corresponding to any one of the full length ngRNAs provided in the Tables.

Any ngRNA exemplified in Tables 35-44 and 46-52 can comprise, or further comprise, a 3′ motif at the 3′ end of the extension arm. In some embodiments, a 3′ motif is capable of forming a tertiary structure on its own, such as a hairpin. The 3′ motif can be connected to the 3′ end of the ngRNA via a linker sequence. The ngRNA may also comprise adaptations at the 3′ end or 5′ end. For example, the ngRNA may comprise a series of 1, 2, 3, 4, 5, 6, 7 or more additional Uracil nucleotides at the 3′ end. In some embodiments, the ngRNA comprises 4 additional Uracil nucleotides at its 3′ end. Without being bound by theory, the 3 motifs or additional Uracil nucleotides can increase PEgRNA stability. ngRNA sequences exemplified in Tables 35-44 and 46-52 may be adapted for expression from a nucleic acid template with a U6 promoter, for example, by including a 5′ terminal G if the spacer of the ngRNA begins with another nucleotide. The modifications and end adaptations included in the selection of full length ngRNAs included in Tables 35-44 and 46-52 are annotated in the third column (“Description”) of the Tables.

The ngRNA exemplified in Tables 35-44 and 46-52 may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond.

TABLE 1

| Sequence | | |
|---|---|---|
| Number | Sequence | Description |
| 1 | TGAGCCACGTGAAGACA (SEQ ID NO: 1) | PEgRNA spacer (GG PAM) |
| 2 | CTGAGCCACGTGAAGACA (SEQ ID NO: 2) | PEgRNA spacer (GG PAM) |
| 3 | ACTGAGCCACGTGAAGACA (SEQ ID NO: 3) | PEgRNA spacer (GG PAM) |
| 4 | AACTGAGCCACGTGAAGACA (SEQ ID NO: 4) | PEgRNA spacer (GG PAM) |
| 5 | GAACTGAGCCACGTGAAGACA (SEQ ID NO: 5) | PEgRNA spacer (GG PAM) |
| 6 | GGAACTGAGCCACGTGAAGACA (SEQ ID NO: 6) | PEgRNA spacer (GG PAM) |
| 7 | CTTCA | PBS |
| 8 | CTTCAC | PBS |
| 9 | CTTCACG | PBS |
| 10 | CTTCACGT | PBS |
| 11 | CTTCACGTG | PBS |
| 12 | CTTCACGTGG (SEQ ID NO: 12) | PBS |
| 13 | CTTCACGTGGC (SEQ ID NO: 13) | PBS |
| 14 | CTTCACGTGGCT (SEQ ID NO: 14) | PBS |
| 15 | CTTCACGTGGCTC (SEQ ID NO: 15) | PBS |
| 16 | CTTCACGTGGCTCA (SEQ ID NO: 16) | PBS |
| 17 | CTTCACGTGGCTCAG (SEQ ID NO: 17) | PBS |
| 18 | CTTCACGTGGCTCAGT (SEQ ID NO: 18) | PBS |
| 19 | CTTCACGTGGCTCAGTT (SEQ ID NO: 19) | PBS |
| 20 | CTTCACGTGGCTCAGTTC (SEQ ID NO: 20) | PBS |
| 21 | CTTCACGTGGCTCAGTTCC (SEQ ID NO: 21) | PBS |
| 22 | GATCAGGCTTCCACTTTGGAAACCCAGCAAGAC | RTT |
| | ACCTGT (SEQ ID NO: 22) | |
| 23 | GGATCAGGCTTCCACTTTGGAAACCCAGCAAGA | RTT |
| | CACCTGT (SEQ ID NO: 23) | |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b ngRNA spacer (GG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b ngRNA spacer (TG PAM) |
| 29 | CCTTTTCGCTTTTTCCAAGA (SEQ ID NO: 29) | PE3 ngRNA spacer (TG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b ngRNA spacer (AG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG PAM) |
| 36 | GGCTTCCACTTTGGAAACCC (SEQ ID NO: 36) | PE3 ngRNA spacer (AG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 40 | TCCACTTTGGAAACCCAGCA (SEQ ID NO: 40) | PE3 ngRNA spacer (AG PAM) |
| 41 | TGATTATCAGTTTTGGATGC (SEQ ID NO: 41) | PE3 ngRNA spacer (AG PAM) |
| 42 | TGGAAACCCAGCAAGACACC (SEQ ID NO: 42) | PE3 ngRNA spacer (TG PAM) |
| 43 | TGGGATCAGGCTTCCACTTT (SEQ ID NO: 43) | PE3b ngRNA spacer (GG PAM) |
| 44 | TTATCAGTTTTGGATGCAGA (SEQ ID NO: 44) | PE3 ngRNA spacer (AG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 60) | |

TABLE 2

| Sequence | | |
|---|---|---|
| Number | Sequence | Description |
| 61 | TGAAGACAGGTGTCTTG (SEQ ID NO: 61) | PEgRNA spacer (CTGG; CTGGGT PAM) |
| 62 | GTGAAGACAGGTGTCTTG (SEQ ID NO: 62) | PEgRNA spacer (CTGG; CTGGGT PAM) |
| 63 | CGTGAAGACAGGTGTCTTG (SEQ ID NO: 63) | PEgRNA spacer (CTGG; CTGGGT PAM) |
| 64 | ACGTGAAGACAGGTGTCTTG (SEQ ID NO: 64) | PEgRNA spacer (CTGG; CTGGGT PAM) |
| 65 | CACGTGAAGACAGGTGTCTTG (SEQ ID NO: 65) | PEgRNA spacer (CTGG; CTGGGT PAM) |
| 66 | CCACGTGAAGACAGGTGTCTTG (SEQ ID NO: 66) | PEgRNA spacer (CTGG; CTGGGT PAM) |
| 67 | GACAC | PBS |
| 68 | GACACC | PBS |
| 69 | GACACCT | PBS |
| 70 | GACACCTG | PBS |
| 71 | GACACCTGT | PBS |
| 72 | GACACCTGTC (SEQ ID NO: 72) | PBS |
| 73 | GACACCTGTCT (SEQ ID NO: 73) | PBS |
| 74 | GACACCTGTCTT (SEQ ID NO: 74) | PBS |
| 75 | GACACCTGTCTTC (SEQ ID NO: 75) | PBS |
| 76 | GACACCTGTCTTCA (SEQ ID NO: 76) | PBS |
| 77 | GACACCTGTCTTCAC (SEQ ID NO: 77) | PBS |
| 78 | GACACCTGTCTTCACG (SEQ ID NO: 78) | PBS |
| 79 | GACACCTGTCTTCACGT (SEQ ID NO: 79) | PBS |
| 80 | GACACCTGTCTTCACGTG (SEQ ID NO: 80) | PBS |
| 81 | GACACCTGTCTTCACGTGG (SEQ ID NO: 81) | PBS |
| 82 | GATCAGGCTTCCACTTTGGAAACACAGCAA (SEQ | RTT (CTGG-to-CTGT; CTGGGT-to- |
| | ID NO: 82) | CTGTGT PAM silencing edit) |
| 83 | GATCAGGCTTCCACTTTGGAAACCCAGCAA (SEQ | RTT |
| | ID NO: 83) | |
| 84 | GATCAGGCTTCCACTTTGGAAACGCAGCAA (SEQ | RTT (CTGG-to-CTGC; CTGGGT-to- |
| | ID NO: 84) | CTGCGT PAM silencing edit) |
| 85 | GATCAGGCTTCCACTTTGGAAACTCAGCAA (SEQ | RTT (CTGG-to-CTGA PAM silencing edit) |
| | ID NO: 85) | |
| 86 | GGATCAGGCTTCCACTTTGGAAACACAGCAA | RTT (CTGG-to-CTGT; CTGGGT-to- |
| | (SEQ ID NO: 86) | CTGTGT PAM silencing edit) |
| 87 | GGATCAGGCTTCCACTTTGGAAACCCAGCAA | RTT |
| | (SEQ ID NO: 87) | |
| 88 | GGATCAGGCTTCCACTTTGGAAACGCAGCAA | RTT (CTGG-to-CTGC; CTGGGT-to- |
| | (SEQ ID NO: 88) | CTGCGT PAM silencing edit) |
| 89 | GGATCAGGCTTCCACTTTGGAAACTCAGCAA | RTT (CTGG-to-CTGA PAM silencing edit) |
| | (SEQ ID NO: 89) | |
| 90 | GGGATCAGGCTTCCACTTTGGAAACACAGCAA | RTT (CTGG-to-CTGT; CTGGGT-to- |
| | (SEQ ID NO: 90) | CTGTGT PAM silencing edit) |
| 91 | GGGATCAGGCTTCCACTTTGGAAACCCAGCAA | RTT |
| | (SEQ ID NO: 91) | |
| 92 | GGGATCAGGCTTCCACTTTGGAAACGCAGCAA | RTT (CTGG-to-CTGC; CTGGGT-to- |
| | (SEQ ID NO: 92) | CTGCGT PAM silencing edit) |
| 93 | GGGATCAGGCTTCCACTTTGGAAACTCAGCAA | RTT (CTGG-to-CTGA PAM silencing edit) |
| | (SEQ ID NO: 93) | |
| 94 | TGGGATCAGGCTTCCACTTTGGAAACACAGCAA | RTT (CTGG-to-CTGT; CTGGGT-to- |
| | (SEQ ID NO: 94) | CTGTGT PAM silencing edit) |
| 95 | TGGGATCAGGCTTCCACTTTGGAAACCCAGCAA | RTT |
| | (SEQ ID NO: 95) | |
| 96 | TGGGATCAGGCTTCCACTTTGGAAACGCAGCAA | RTT (CTGG-to-CTGC; CTGGGT-to- |
| | (SEQ ID NO: 96) | CTGCGT PAM silencing edit) |
| 97 | TGGGATCAGGCTTCCACTTTGGAAACTCAGCAA | RTT (CTGG-to-CTGA PAM silencing edit) |
| | (SEQ ID NO: 97) | |
| 98 | ATGGGATCAGGCTTCCACTTTGGAAACACAGCA | RTT (CTGG-to-CTGT; CTGGGT-to- |
| | A (SEQ ID NO: 98) | CTGTGT PAM silencing edit) |
| 99 | ATGGGATCAGGCTTCCACTTTGGAAACCCAGCA | RTT |
| | A (SEQ ID NO: 99) | |
| 100 | ATGGGATCAGGCTTCCACTTTGGAAACGCAGCA | RTT (CTGG-to-CTGC; CTGGGT-to- |
| | A (SEQ ID NO: 100) | CTGCGT PAM silencing edit) |
| 101 | ATGGGATCAGGCTTCCACTTTGGAAACTCAGCAA | RTT (CTGG-to-CTGA PAM silencing edit) |
| | (SEQ ID NO: 101) | |
| 102 | TATGGGATCAGGCTTCCACTTTGGAAACACAGCA | RTT (CTGG-to-CTGT; CTGGGT-to- |
| | A (SEQ ID NO: 102) | CTGTGT PAM silencing edit) |
| 103 | TATGGGATCAGGCTTCCACTTTGGAAACCCAGCA | RTT |
| | A (SEQ ID NO: 103) | |
| 104 | TATGGGATCAGGCTTCCACTTTGGAAACGCAGCA | RTT (CTGG-to-CTGC; CTGGGT-to- |
| | A (SEQ ID NO: 104) | CTGCGT PAM silencing edit) |
| 105 | TATGGGATCAGGCTTCCACTTTGGAAACTCAGCA | RTT (CTGG-to-CTGA PAM silencing edit) |
| | A (SEQ ID NO: 105) | |
| 106 | GTATGGGATCAGGCTTCCACTTTGGAAACACAGC | RTT (CTGG-to-CTGT; CTGGGT-to- |
| | AA (SEQ ID NO: 106) | CTGTGT PAM silencing edit) |
| 107 | GTATGGGATCAGGCTTCCACTTTGGAAACCCAGC | RTT |
| | AA (SEQ ID NO: 107) | |
| 108 | GTATGGGATCAGGCTTCCACTTTGGAAACGCAGC | RTT (CTGG-to-CTGC; CTGGGT-to- |
| | AA (SEQ ID NO: 108) | CTGCGT PAM silencing edit) |
| 109 | GTATGGGATCAGGCTTCCACTTTGGAAACTCAGC | RTT (CTGG-to-CTGA PAM silencing edit) |
| | AA (SEQ ID NO: 109) | |
| 110 | TGTATGGGATCAGGCTTCCACTTTGGAAACACAG | RTT (CTGG-to-CTGT; CTGGGT-to- |
| | CAA (SEQ ID NO: 110) | CTGTGT PAM silencing edit) |
| 111 | TGTATGGGATCAGGCTTCCACTTTGGAAACCCAG | RTT |
| | CAA (SEQ ID NO: 111) | |
| 112 | TGTATGGGATCAGGCTTCCACTTTGGAAACGCAG | RTT (CTGG-to-CTGC; CTGGGT-to- |
| | CAA (SEQ ID NO: 112) | CTGCGT PAM silencing edit) |
| 113 | TGTATGGGATCAGGCTTCCACTTTGGAAACTCAG | RTT (CTGG-to-CTGA PAM silencing edit) |
| | CAA (SEQ ID NO: 113) | |
| 114 | CTGTATGGGATCAGGCTTCCACTTTGGAAACACA | RTT (CTGG-to-CTGT; CTGGGT-to- |
| | GCAA (SEQ ID NO: 114) | CTGTGT PAM silencing edit) |
| 115 | CTGTATGGGATCAGGCTTCCACTTTGGAAACCCA | RTT |
| | GCAA (SEQ ID NO: 115) | |
| 116 | CTGTATGGGATCAGGCTTCCACTTTGGAAACGCA | RTT (CTGG-to-CTGC; CTGGGT-to- |
| | GCAA (SEQ ID NO: 116) | CTGCGT PAM silencing edit) |
| 117 | CTGTATGGGATCAGGCTTCCACTTTGGAAACTCA | RTT (CTGG-to-CTGA PAM silencing edit) |
| | GCAA (SEQ ID NO: 117) | |
| 118 | TCTGTATGGGATCAGGCTTCCACTTTGGAAACAC | RTT (CTGG-to-CTGT; CTGGGT-to- |
| | AGCAA (SEQ ID NO: 118) | CTGTGT PAM silencing edit) |
| 119 | TCTGTATGGGATCAGGCTTCCACTTTGGAAACCC | RTT |
| | AGCAA (SEQ ID NO: 119) | |
| 120 | TCTGTATGGGATCAGGCTTCCACTTTGGAAACGC | RTT (CTGG-to-CTGC; CTGGGT-to- |
| | AGCAA (SEQ ID NO: 120) | CTGCGT PAM silencing edit) |
| 121 | TCTGTATGGGATCAGGCTTCCACTTTGGAAACTC | RTT (CTGG-to-CTGA PAM silencing edit) |
| | AGCAA (SEQ ID NO: 121) | |
| 122 | TTCTGTATGGGATCAGGCTTCCACTTTGGAAACA | RTT (CTGG-to-CTGT; CTGGGT-to- |
| | CAGCAA (SEQ ID NO: 122) | CTGTGT PAM silencing edit) |
| 123 | TTCTGTATGGGATCAGGCTTCCACTTTGGAAACC | RTT |
| | CAGCAA (SEQ ID NO: 123) | |
| 124 | TTCTGTATGGGATCAGGCTTCCACTTTGGAAACG | RTT (CTGG-to-CTGC; CTGGGT-to- |
| | CAGCAA (SEQ ID NO: 124) | CTGCGT PAM silencing edit) |
| 125 | TTCTGTATGGGATCAGGCTTCCACTTTGGAAACT | RTT (CTGG-to-CTGA PAM silencing edit) |
| | CAGCAA (SEQ ID NO: 125) | |
| 25 | AAGATTCAGTAGATCTTTCT (SEQ ID NO: 25) | PE3 ngRNA spacer (TGTGAT PAM) |
| 126 | ACCTTTTCGCTTTTTCCAAG (SEQ ID NO: 126) | PE3 ngRNA spacer (ATGG PAM) |
| 127 | AGAAGCTTTCTGTATGGGAT (SEQ ID NO: 127) | PE3b ngRNA spacer (CAGG PAM) |
| 128 | AGTAGATCTTTCTTGTGATT (SEQ ID NO: 128) | PE3 ngRNA spacer (ATCAGT PAM) |
| 129 | CATCACCTTTTCGCTTTTTC (SEQ ID NO: 129) | PE3 ngRNA spacer (CAAGAT PAM) |
| 130 | CCAAGATGGCTCAAGATTCA (SEQ ID NO: 130) | PE3 ngRNA spacer (GTAGAT PAM) |
| 131 | CTTTCTTGTGATTATCAGTT (SEQ ID NO: 131) | PE3 ngRNA spacer (TTGG; TTGGAT PAM) |
| 132 | TATGGGATCAGGCTTCCACT (SEQ ID NO: 132) | PE3b ngRNA spacer (TTGG PAM) |
| 133 | TCGCTTTTTCCAAGATGGCT (SEQ ID NO: 133) | PE3 ngRNA spacer (CAAGAT PAM) |
| 134 | TGGATGCAGAAGCTTTCTGT (SEQ ID NO: 134) | PE3 ngRNA spacer (ATGG; ATGGAT PAM) |
| 135 | TTTTCCAAGATGGCTCAAGA (SEQ ID NO: 135) | PE3 ngRNA spacer (TTCAGT PAM) |

TABLE 3

| Sequence | | |
|---|---|---|
| Number | Sequence | Description |
| 136 | AGCTTTCTGTATGGATC (SEQ ID NO: 136) | PEgRNA spacer (AG; AGG PAM) |
| 137 | AAGCTTTCTGTATGGATC (SEQ ID NO: 137) | PEgRNA spacer (AG; AGG PAM) |
| 138 | GAAGCTTTCTGTATGGATC (SEQ ID NO: 138) | PEgRNA spacer (AG; AGG PAM) |
| 139 | AGAAGCTTTCTGTATGGATC (SEQ ID NO: 139) | PEgRNA spacer (AG; AGG PAM) |
| 140 | CAGAAGCTTTCTGTATGGATC (SEQ ID NO: 140) | PEgRNA spacer (AG; AGG PAM) |
| 141 | GCAGAAGCTTTCTGTATGGATC (SEQ ID NO: 141) | PEgRNA spacer (AG; AGG PAM) |
| 142 | CCATA | PBS |
| 143 | CCATAC | PBS |
| 144 | CCATACA | PBS |
| 145 | CCATACAG | PBS |
| 146 | CCATACAGA | PBS |
| 147 | CCATACAGAA (SEQ ID NO: 147) | PBS |
| 148 | CCATACAGAAA (SEQ ID NO: 148) | PBS |
| 149 | CCATACAGAAAG (SEQ ID NO: 149) | PBS |
| 150 | CCATACAGAAAGC (SEQ ID NO: 150) | PBS |
| 151 | CCATACAGAAAGCT (SEQ ID NO: 151) | PBS |
| 152 | CCATACAGAAAGCTT (SEQ ID NO: 152) | PBS |
| 153 | CCATACAGAAAGCTTC (SEQ ID NO: 153) | PBS |
| 154 | CCATACAGAAAGCTTCT (SEQ ID NO: 154) | PBS |
| 155 | CCATACAGAAAGCTTCTG (SEQ ID NO: 155) | PBS |
| 156 | CCATACAGAAAGCTTCTGC (SEQ ID NO: 156) | PBS |
| 157 | AAGCCTGATC (SEQ ID NO: 157) | RTT |
| 158 | AAGCTTGATC (SEQ ID NO: 158) | RTT (AG-to-AA; AGG-to-AAG PAM |
| | | silencing edit) |
| 159 | GAAGCCTGATC (SEQ ID NO: 159) | RTT |
| 160 | GGAAGCCTGATC (SEQ ID NO: 160) | RTT |
| 161 | GGAAGCTTGATC (SEQ ID NO: 161) | RTT (AG-to-AA; AGG-to-AAG PAM |
| | | silencing edit) |
| 162 | TGGAAGCCTGATC (SEQ ID NO: 162) | RTT |
| 163 | GTGGAAGCCTGATC (SEQ ID NO: 163) | RTT |
| 164 | GTGGAAGCTTGATC (SEQ ID NO: 164) | RTT (AG-to-AA; AGG-to-AAG PAM |
| | | silencing edit) |
| 165 | AGTGGAAGCCTGATC (SEQ ID NO: 165) | RTT |
| 166 | AAGTGGAAGCCTGATC (SEQ ID NO: 166) | RTT |
| 167 | AAGTGGAAGCTTGATC (SEQ ID NO: 167) | RTT (AG-to-AA; AGG-to-AAG PAM |
| | | silencing edit) |
| 168 | AAAGTGGAAGCCTGATC (SEQ ID NO: 168) | RTT |
| 169 | CAAAGTGGAAGCCTGATC (SEQ ID NO: 169) | RTT |
| 170 | CCAAAGTGGAAGCCTGATC (SEQ ID NO: 170) | RTT |
| 171 | TCCAAAGTGGAAGCCTGATC (SEQ ID NO: 171) | RTT |
| 172 | TTCCAAAGTGGAAGCCTGATC (SEQ ID NO: 172) | RTT |
| 173 | TTTCCAAAGTGGAAGCCTGATC (SEQ ID NO: 173) | RTT |
| 174 | GTTTCCAAAGTGGAAGCCTGATC (SEQ ID NO: | RTT |
| | 174) | |
| 175 | GGTTTCCAAAGTGGAAGCCTGATC (SEQ ID NO: | RTT |
| | 175) | |
| 176 | GGGTTTCCAAAGTGGAAGCCTGATC (SEQ ID NO: | RTT |
| | 176) | |
| 177 | TGGGTTTCCAAAGTGGAAGCCTGATC (SEQ ID | RTT |
| | NO: 177) | |
| 178 | CTGGGTTTCCAAAGTGGAAGCCTGATC (SEQ ID | RTT |
| | NO: 178) | |
| 179 | GCTGGGTTTCCAAAGTGGAAGCCTGATC (SEQ ID | RTT |
| | NO: 179) | |
| 180 | TGCTGGGTTTCCAAAGTGGAAGCCTGATC (SEQ | RTT |
| | ID NO: 180) | |
| 181 | TTGCTGGGTTTCCAAAGTGGAAGCCTGATC (SEQ | RTT |
| | ID NO: 181) | |
| 182 | TTGCTGGGTTTCCAAAGTGGAAGCTTGATC (SEQ | RTT (AG-to-AA; AGG-to-AAG PAM |
| | ID NO: 182) | silencing edit) |
| 183 | CTTGCTGGGTTTCCAAAGTGGAAGCCTGATC | RTT |
| | (SEQ ID NO: 183) | |
| 184 | TCTTGCTGGGTTTCCAAAGTGGAAGCCTGATC | RTT |
| | (SEQ ID NO: 184) | |
| 185 | TCTTGCTGGGTTTCCAAAGTGGAAGCTTGATC | RTT (AG-to-AA; AGG-to-AAG PAM |
| | (SEQ ID NO: 185) | silencing edit) |
| 186 | GTCTTGCTGGGTTTCCAAAGTGGAAGCCTGATC | RTT |
| | (SEQ ID NO: 186) | |
| 187 | TGTCTTGCTGGGTTTCCAAAGTGGAAGCCTGATC | RTT |
| | (SEQ ID NO: 187) | |
| 188 | TGTCTTGCTGGGTTTCCAAAGTGGAAGCTTGATC | RTT (AG-to-AA; AGG-to-AAG PAM |
| | (SEQ ID NO: 188) | silencing edit) |
| 189 | GTGTCTTGCTGGGTTTCCAAAGTGGAAGCCTGAT | RTT |
| | C (SEQ ID NO: 189) | |
| 190 | GGTGTCTTGCTGGGTTTCCAAAGTGGAAGCCTGA | RTT |
| | TC (SEQ ID NO: 190) | |
| 191 | AGGTGTCTTGCTGGGTTTCCAAAGTGGAAGCCTG | RTT |
| | ATC (SEQ ID NO: 191) | |
| 192 | CAGGTGTCTTGCTGGGTTTCCAAAGTGGAAGCCT | RTT |
| | GATC (SEQ ID NO: 192) | |
| 193 | ACAGGTGTCTTGCTGGGTTTCCAAAGTGGAAGCC | RTT |
| | TGATC (SEQ ID NO: 193) | |
| 194 | GACAGGTGTCTTGCTGGGTTTCCAAAGTGGAAGC | RTT |
| | CTGATC (SEQ ID NO: 194) | |
| 4 | AACTGAGCCACGTGAAGACA (SEQ ID NO: 4) | PE3 ngRNA spacer (GG PAM) |
| 195 | ACATCTTCCTTAGGAACTCC (SEQ ID NO: 195) | PE3 ngRNA spacer (TG; TGG PAM) |
| 196 | ACTACTTACCATCTCTTTCA (SEQ ID NO: 196) | PE3 ngRNA spacer (AG; AGG PAM) |
| 197 | ACTCCTGGAACTGAGCCACG (SEQ ID NO: 197) | PE3 ngRNA spacer (TG PAM) |
| 198 | AGAAAATAATTTCATTATTC (SEQ ID NO: 198) | PE3 ngRNA spacer (TG; TGG PAM) |
| 199 | AGGCTTCATACATCTTCCTT (SEQ ID NO: 199) | PE3 ngRNA spacer (AG; AGG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG; TGG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 207 | GAAAATAATTTCATTATTCT (SEQ ID NO: 207) | PE3 ngRNA spacer (GG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG; AGG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG; TGG PAM) |
| 213 | GTGAAGACAGGTGTCTTGCT (SEQ ID NO: 213) | PE3 ngRNA spacer (GG; GGG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 219 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAG (SEQ ID NO: 219) | |
| 220 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGCT | |
| | TGATCCCATACAG (SEQ ID NO: 220) | |
| 221 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAA (SEQ ID NO: 221) | |
| 222 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAG (SEQ ID NO: 222) | |
| 223 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGCT | |
| | TGATCCCATACAGAA (SEQ ID NO: 223) | |
| 224 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGGAAG | |
| | CTTGATCCCATACAG (SEQ ID NO: 224) | |
| 225 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAG (SEQ ID NO: 225) | |
| 226 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAA (SEQ ID NO: 226) | |
| 227 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAG (SEQ ID NO: 227) | |
| 228 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGTTTT (SEQ ID NO: 228) | |
| 229 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGCT | |
| | TGATCCCATACAGTTTT (SEQ ID NO: 229) | |
| 230 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGCT | |
| | TGATCCCATACAGAAAG (SEQ ID NO: 230) | |
| 231 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGGAAG | |
| | CTTGATCCCATACAGAA (SEQ ID NO: 231) | |
| 232 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGTGGA | |
| | AGCTTGATCCCATACAG (SEQ ID NO: 232) | |
| 233 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGC | |
| | CTGATCCCATACAGTTTT (SEQ ID NO: 233) | |
| 234 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCT (SEQ ID NO: 234) | |
| 235 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAG (SEQ ID NO: 235) | |
| 236 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAA (SEQ ID NO: 236) | |
| 237 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAG (SEQ ID NO: 237) | |
| 238 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAATTTT (SEQ ID NO: 238) | |
| 239 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGTTTT (SEQ ID NO: 239) | |
| 240 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGCT | |
| | TGATCCCATACAGAATTTT (SEQ ID NO: 240) | |
| 241 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGGAAG | |
| | CTTGATCCCATACAGTTTT (SEQ ID NO: 241) | |
| 242 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGCT | |
| | TGATCCCATACAGAAAGCT (SEQ ID NO: 242) | |
| 243 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGGAAG | |
| | CTTGATCCCATACAGAAAG (SEQ ID NO: 243) | |
| 244 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGTGGA | |
| | AGCTTGATCCCATACAGAA (SEQ ID NO: 244) | |
| 245 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGTG | |
| | GAAGCTTGATCCCATACAG (SEQ ID NO: 245) | |
| 246 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGC | |
| | CTGATCCCATACAGAATTTT (SEQ ID NO: 246) | |
| 247 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGGAA | |
| | GCCTGATCCCATACAGTTTT (SEQ ID NO: 247) | |
| 248 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCT (SEQ ID NO: 248) | |
| 249 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAG (SEQ ID NO: 249) | |
| 250 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGAA (SEQ ID NO: 250) | |
| 251 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGTTTT (SEQ ID NO: 251) | |
| 252 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAATTTT (SEQ ID NO: 252) | |
| 253 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGTTTT (SEQ ID NO: 253) | |
| 254 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGCT | |
| | TGATCCCATACAGAAAGTTTT (SEQ ID NO: 254) | |
| 255 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGGAAG | |
| | CTTGATCCCATACAGAATTTT (SEQ ID NO: 255) | |
| 256 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGTGGA | |
| | AGCTTGATCCCATACAGTTTT (SEQ ID NO: 256) | |
| 257 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGGAAG | |
| | CTTGATCCCATACAGAAAGCT (SEQ ID NO: 257) | |
| 258 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGTGGA | |
| | AGCTTGATCCCATACAGAAAG (SEQ ID NO: 258) | |
| 259 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGTG | |
| | GAAGCTTGATCCCATACAGAA (SEQ ID NO: 259) | |
| 260 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGC | |
| | CTGATCCCATACAGAAAGTTTT (SEQ ID NO: 260) | |
| 261 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGGAA | |
| | GCCTGATCCCATACAGAATTTT (SEQ ID NO: 261) | |
| 262 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGTGG | |
| | AAGCCTGATCCCATACAGTTTT (SEQ ID NO: 262) | |
| 263 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCT (SEQ ID NO: | |
| | 263) | |
| 264 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGAAAG (SEQ ID NO: | |
| | 264) | |
| 265 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTTTT (SEQ ID | |
| | NO: 265) | |
| 266 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGTTTT (SEQ ID NO: | |
| | 266) | |
| 267 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAATTTT (SEQ ID NO: | |
| | 267) | |
| 268 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGTTTT (SEQ ID NO: | |
| | 268) | |
| 269 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGCT | |
| | TGATCCCATACAGAAAGCTTTTT (SEQ ID | |
| | NO: 269) | |
| 270 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGGAAG | |
| | CTTGATCCCATACAGAAAGTTTT (SEQ ID | |
| | NO: 270) | |
| 271 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGTGGA | |
| | AGCTTGATCCCATACAGAATTTT (SEQ ID | |
| | NO: 271) | |
| 272 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGTG | |
| | GAAGCTTGATCCCATACAGTTTT (SEQ ID | |
| | NO: 272) | |
| 273 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCGTGGA | |
| | AGCTTGATCCCATACAGAAAGCT (SEQ ID NO: | |
| | 273) | |
| 274 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGTG | |
| | GAAGCTTGATCCCATACAGAAAG (SEQ ID NO: | |
| | 274) | |
| 275 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGC | |
| | CTGATCCCATACAGAAAGCTTTTT (SEQ ID NO: | |
| | 275) | |
| 276 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGGAA | |
| | GCCTGATCCCATACAGAAAGTTTT (SEQ ID NO: | |
| | 276) | |
| 277 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGTGG | |
| | AAGCCTGATCCCATACAGAATTTT (SEQ ID NO: | |
| | 277) | |
| 278 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGT | |
| | GGAAGCCTGATCCCATACAGTTTT (SEQ ID NO: | |
| | 278) | |
| 279 | AGAAGCTTTCTGTATGGATCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGAAAGCT (SEQ ID NO: | |
| | 279) | |
| 280 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTTTT (SEQ ID NO: | |
| | 280) | |
| 281 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGTTTT (SEQ ID NO: | |
| | 281) | |
| 282 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGAATTTT (SEQ ID NO: | |
| | 282) | |
| 283 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGGAAG | |
| | CTTGATCCCATACAGAAAGCTTTTT (SEQ ID NO: | |
| | 283) | |
| 284 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGTGGA | |
| | AGCTTGATCCCATACAGAAAGTTTT (SEQ ID NO: | |
| | 284) | |
| 285 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGTG | |
| | GAAGCTTGATCCCATACAGAATTTT (SEQ ID NO: | |
| | 285) | |
| 286 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCAAGTG | |
| | GAAGCTTGATCCCATACAGAAAGCT (SEQ ID NO: | |
| | 286) | |
| 287 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGGAA | |
| | GCCTGATCCCATACAGAAAGCTTTTT (SEQ ID NO: | |
| | 287) | |
| 288 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGTGG | |
| | AAGCCTGATCCCATACAGAAAGTTTT (SEQ ID | |
| | NO: 288) | |
| 289 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGT | |
| | GGAAGCCTGATCCCATACAGAATTTT (SEQ ID | |
| | NO: 289) | |
| 290 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTTTT (SEQ ID | |
| | NO: 290) | |
| 291 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGAAAGTTTT (SEQ ID | |
| | NO: 291) | |
| 292 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCGTGGA | |
| | AGCTTGATCCCATACAGAAAGCTTTTT (SEQ ID | |
| | NO: 292) | |
| 293 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGTG | |
| | GAAGCTTGATCCCATACAGAAAGTTTT (SEQ ID | |
| | NO: 293) | |
| 294 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCGTGG | |
| | AAGCCTGATCCCATACAGAAAGCTTTTT (SEQ ID | |
| | NO: 294) | |
| 295 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGT | |
| | GGAAGCCTGATCCCATACAGAAAGTTTT (SEQ ID | |
| | NO: 295) | |
| 296 | AGAAGCTTTCTGTATGGATCGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGTG | |
| | GAAGCCTGATCCCATACAGAAAGCTTTTT (SEQ | |
| | ID NO: 296) | |
| 297 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit), contains 3′ TTTT |
| | ACTTGAAAAAGTGGCACCGAGTCGGTGCAAGTG | |
| | GAAGCTTGATCCCATACAGAAAGCTTTTT (SEQ | |
| | ID NO: 297) | |
| 298 | GAGAAGCTTTCTGTATGGATCGTTTTAGAGCTAG | pegRNA, contains 3′ TTTT; 5′G |
| | AAATAGCAAGTTAAAATAAGGCTAGTCCGTTATC | |
| | AACTTGAAAAAGTGGCACCGAGTCGGTGCAAGT | |
| | GGAAGCCTGATCCCATACAGAAAGCTTTTT (SEQ | |
| | ID NO: 298) | |
| 299 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTTGCTG | |
| | GGTTTCCAAAGTGGAAGCTTGATCCCATACAG | |
| | (SEQ ID NO: 299) | |
| 300 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTTGCTG | |
| | GGTTTCCAAAGTGGAAGCTTGATCCCATACAGAA | |
| | (SEQ ID NO: 300) | |
| 301 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTCTTGC | |
| | TGGGTTTCCAAAGTGGAAGCTTGATCCCATACAG | |
| | (SEQ ID NO: 301) | |
| 302 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTTGCTG | |
| | GGTTTCCAAAGTGGAAGCTTGATCCCATACAGAA | |
| | AG (SEQ ID NO: 302) | |
| 303 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTCTTGC | |
| | TGGGTTTCCAAAGTGGAAGCTTGATCCCATACAG | |
| | AA (SEQ ID NO: 303) | |
| 304 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTGTCTT | |
| | GCTGGGTTTCCAAAGTGGAAGCTTGATCCCATAC | |
| | AG (SEQ ID NO: 304) | |
| 305 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTTGCTG | |
| | GGTTTCCAAAGTGGAAGCTTGATCCCATACAGAA | |
| | AGCT (SEQ ID NO: 305) | |
| 306 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTCTTGC | |
| | TGGGTTTCCAAAGTGGAAGCTTGATCCCATACAG | |
| | AAAG (SEQ ID NO: 306) | |
| 307 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTGTCTT | |
| | GCTGGGTTTCCAAAGTGGAAGCTTGATCCCATAC | |
| | AGAA (SEQ ID NO: 307) | |
| 308 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTCTTGC | |
| | TGGGTTTCCAAAGTGGAAGCTTGATCCCATACAG | |
| | AAAGCT (SEQ ID NO: 308) | |
| 309 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTGTCTT | |
| | GCTGGGTTTCCAAAGTGGAAGCTTGATCCCATAC | |
| | AGAAAG (SEQ ID NO: 309) | |
| 310 | AGAAGCTTTCTGTATGGATCGTTTTAGAGCTAGA | pegRNA (AG-to-AA; AGG-to-AAG PAM |
| | AATAGCAAGTTAAAATAAGGCTAGTCCGTTATCA | silencing edit) |
| | ACTTGAAAAAGTGGCACCGAGTCGGTGCTGTCTT | |
| | GCTGGGTTTCCAAAGTGGAAGCTTGATCCCATAC | |
| | AGAAAGCT (SEQ ID NO: 310) | |
| 311 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTTGCT | AACATTGA; hairpin SEQ ID NO: 3591; 3′ |
| | GGGTTTCCAAAGTGGAAGCTTGATCCCATACAGA | TTTT; 5′G |
| | ACATTGACGCGTCTCTACGTGGGGGCGCGTTTT | |
| | (SEQ ID NO: 311) | |
| 312 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTTGCT | AACATTGA; hairpin SEQ ID NO: 3591; 3′ |
| | GGGTTTCCAAAGTGGAAGCTTGATCCCATACAGA | TTTT; 5′G |
| | AAACATTGACGCGTCTCTACGTGGGGGCGCGTTT | |
| | T (SEQ ID NO: 312) | |
| 313 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTCTTG | AACATTGA; hairpin SEQ ID NO: 3591; 3′ |
| | CTGGGTTTCCAAAGTGGAAGCTTGATCCCATACA | TTTT; 5′G |
| | GAACATTGACGCGTCTCTACGTGGGGGCGCGTTT | |
| | T (SEQ ID NO: 313) | |
| 314 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTTGCT | AACATTGA; hairpin SEQ ID NO: 3591; 3′ |
| | GGGTTTCCAAAGTGGAAGCTTGATCCCATACAGA | TTTT; 5′G |
| | AAGAACATTGACGCGTCTCTACGTGGGGGCGCG | |
| | TTTT (SEQ ID NO: 314) | |
| 315 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTCTTG | AACATTGA; hairpin SEQ ID NO: 3591; 3′ |
| | CTGGGTTTCCAAAGTGGAAGCTTGATCCCATACA | TTTT; 5′G |
| | GAAAACATTGACGCGTCTCTACGTGGGGGCGCG | |
| | TTTT (SEQ ID NO: 315) | |
| 316 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTGTCT | AACATTGA; hairpin SEQ ID NO: 3591; 3′ |
| | TGCTGGGTTTCCAAAGTGGAAGCTTGATCCCATA | TTTT; 5′G |
| | CAGAACATTGACGCGTCTCTACGTGGGGGCGCGT | |
| | TTT (SEQ ID NO: 316) | |
| 317 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTTGCT | AACATTGA; hairpin SEQ ID NO: 3591; 3′ |
| | GGGTTTCCAAAGTGGAAGCTTGATCCCATACAGA | TTTT; 5′G |
| | AAGCTAACATTGACGCGTCTCTACGTGGGGGCGC | |
| | GTTTT (SEQ ID NO: 317) | |
| 318 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTCTTG | AACATTGA; hairpin SEQ ID NO: 3591; 3′ |
| | CTGGGTTTCCAAAGTGGAAGCTTGATCCCATACA | TTTT; 5′G |
| | GAAAGAACATTGACGCGTCTCTACGTGGGGGCG | |
| | CGTTTT (SEQ ID NO: 318) | |
| 319 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTGTCT | AACATTGA; hairpin SEQ ID NO: 3591; 3′ |
| | TGCTGGGTTTCCAAAGTGGAAGCTTGATCCCATA | TTTT; 5′G |
| | CAGAAAACATTGACGCGTCTCTACGTGGGGGCG | |
| | CGTTTT (SEQ ID NO: 319) | |
| 320 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTCTTG | AACATTGA; hairpin SEQ ID NO: 3591; 3′ |
| | CTGGGTTTCCAAAGTGGAAGCTTGATCCCATACA | TTTT; 5′G |
| | GAAAGCTAACATTGACGCGTCTCTACGTGGGGG | |
| | CGCGTTTT (SEQ ID NO: 320) | |
| 321 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTGTCT | AACATTGA; hairpin SEQ ID NO: 3591; 3′ |
| | TGCTGGGTTTCCAAAGTGGAAGCTTGATCCCATA | TTTT; 5′G |
| | CAGAAAGAACATTGACGCGTCTCTACGTGGGGG | |
| | CGCGTTTT (SEQ ID NO: 321) | |
| 322 | GAGAAGCTTTCTGTATGGATCGTTTTAGAGCTAG | pegRNA (AG-to-AA; AGG-to-AAG PAM |
| | AAATAGCAAGTTAAAATAAGGCTAGTCCGTTATC | silencing edit), contains linker sequence |
| | AACTTGAAAAAGTGGCACCGAGTCGGTGCTGTCT | AACATTGA; hairpin SEQ ID NO: 3591; 3′ |
| | TGCTGGGTTTCCAAAGTGGAAGCTTGATCCCATA | TTTT; 5′G |
| | CAGAAAGCTAACATTGACGCGTCTCTACGTGGG | |
| | GGCGCGTTTT (SEQ ID NO: 322) | |
| 323 | GTCTTGCTGGGTTTCCAAAGGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG; GGG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG; AGG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG; TGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG; GGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG; AGG PAM), contains 3′ |
| | AAATAGCAAGTTAAAATAAGGCTAGTCCGTTATC | TTTT |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG; TGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG; TGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG; AGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG; AGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG; TGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (GG; GGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 343) | |
| 3592 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTTGCTG | silencing edit), contains linker sequence |
| | GGTTTCCAAAGTGGAAGCTTGATCCCATACAGAA | AACATTGA; hairpin SEQ ID NO: 3591 |
| | CATTGACGCGTCTCTACGTGGGGGCGCG (SEQ ID | |
| | NO: 3592) | |
| 3593 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PERNA spacer PAM |
| | ACTTGAAAAAGTGGCACCGAGTCGGTGCTTGCTG | silencing edit), contains linker sequence |
| | GGTTTCCAAAGTGGAAGCTTGATCCCATACAGAA | AACATTGA; hairpin SEQ ID NO: 3591 |
| | AACATTGACGCGTCTCTACGTGGGGGCGCG (SEQ | |
| | ID NO: 3593) | |
| 3594 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTCTTGC | silencing edit), contains linker sequence |
| | TGGGTTTCCAAAGTGGAAGCTTGATCCCATACAG | AACATTGA; hairpin SEQ ID NO: 3591 |
| | AACATTGACGCGTCTCTACGTGGGGGCGCG (SEQ | |
| | ID NO: 3594) | |
| 3595 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTTGCTG | silencing edit), contains linker sequence |
| | GGTTTCCAAAGTGGAAGCTTGATCCCATACAGAA | AACATTGA; hairpin SEQ ID NO: 3591 |
| | AGAACATTGACGCGTCTCTACGTGGGGGCGCG | |
| | (SEQ ID NO: 3595) | |
| 3596 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTCTTGC | silencing edit), contains linker sequence |
| | TGGGTTTCCAAAGTGGAAGCTTGATCCCATACAG | AACATTGA; hairpin SEQ ID NO: 3591 |
| | AAAACATTGACGCGTCTCTACGTGGGGGCGCG | |
| | (SEQ ID NO: 3596) | |
| 3597 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTGTCTT | silencing edit), contains linker sequence |
| | GCTGGGTTTCCAAAGTGGAAGCTTGATCCCATAC | AACATTGA; hairpin SEQ ID NO: 3591 |
| | AGAACATTGACGCGTCTCTACGTGGGGGCGCG | |
| | (SEQ ID NO: 3597) | |
| 3598 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTTGCTG | silencing edit), contains linker sequence |
| | GGTTTCCAAAGTGGAAGCTTGATCCCATACAGAA | AACATTGA; hairpin SEQ ID NO: 3591 |
| | AGCTAACATTGACGCGTCTCTACGTGGGGGCGCG | |
| | (SEQ ID NO: 3598) | |
| 3599 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PERNA spacer PAM |
| | ACTTGAAAAAGTGGCACCGAGTCGGTGCTCTTGC | silencing edit), contains linker sequence |
| | TGGGTTTCCAAAGTGGAAGCTTGATCCCATACAG | AACATTGA; hairpin SEQ ID NO: 3591 |
| | AAAGAACATTGACGCGTCTCTACGTGGGGGCGC | |
| | G (SEQ ID NO: 3599) | |
| 3600 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTGTCTT | silencing edit), contains linker sequence |
| | GCTGGGTTTCCAAAGTGGAAGCTTGATCCCATAC | AACATTGA; hairpin SEQ ID NO: 3591 |
| | AGAAAACATTGACGCGTCTCTACGTGGGGGCGC | |
| | G (SEQ ID NO: 3600) | |
| 3601 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTCTTGC | silencing edit), contains linker sequence |
| | TGGGTTTCCAAAGTGGAAGCTTGATCCCATACAG | AACATTGA; hairpin SEQ ID NO: 3591 |
| | AAAGCTAACATTGACGCGTCTCTACGTGGGGGC | |
| | GCG (SEQ ID NO: 3601) | |
| 3602 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PERNA spacer PAM |
| | ACTTGAAAAAGTGGCACCGAGTCGGTGCTGTCTT | silencing edit), contains linker sequence |
| | GCTGGGTTTCCAAAGTGGAAGCTTGATCCCATAC | AACATTGA; hairpin SEQ ID NO: 3591 |
| | AGAAAGAACATTGACGCGTCTCTACGTGGGGGC | |
| | GCG (SEQ ID NO: 3602) | |
| 3603 | AGAAGCTTTCTGTATGGATCGTTTTAGAGCTAGA | pegRNA (AG-to-AA; AGG-to-AAG for |
| | AATAGCAAGTTAAAATAAGGCTAGTCCGTTATCA | pathogenic only PEgRNA spacer PAM |
| | ACTTGAAAAAGTGGCACCGAGTCGGTGCTGTCTT | silencing edit), contains linker sequence |
| | GCTGGGTTTCCAAAGTGGAAGCTTGATCCCATAC | AACATTGA; hairpin SEQ ID NO: 3591 |
| | AGAAAGCTAACATTGACGCGTCTCTACGTGGGG | |
| | GCGCG (SEQ ID NO: 3603) | |
| 3604 | GTCTTGCTGGGTTTCCAAAGGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3604) | |
| 3605 | GTGAAGACAGGTGTCTTGCTGTTTTAGAGCTAGA | PE3 ngRNA (GG; GGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3605) | |
| 3606 | GAACTGAGCCACGTGAAGACGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591 |
| | TTGACGCGTCTCTACGTGGGGGCGCG (SEQ ID | |
| | NO: 3606) | |
| 3607 | CGTGAAGACAGGTGTCTTGCGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3607) | |
| 3608 | ACATCTTCCTTAGGAACTCCGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3608) | |
| 3609 | AGGCTTCATACATCTTCCTTGTTTTAGAGCTAGA | PE3 ngRNA (AG; AGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3609) | |
| 3610 | ACTACTTACCATCTCTTTCAGTTTTAGAGCTAGA | PE3 ngRNA (AG; AGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3610) | |

TABLE 4

| Sequence | | |
|---|---|---|
| Number | Sequence | Description |
| 344 | TCTTTCTTGTGATTATC (SEQ ID NO: 344) | PERNA spacer (AG PAM) |
| 345 | ATCTTTCTTGTGATTATC (SEQ ID NO: 345) | PERNA spacer (AG PAM) |
| 346 | GATCTTTCTTGTGATTATC (SEQ ID NO: 346) | PERNA spacer (AG PAM) |
| 27 | AGATCTTTCTTGTGATTATC (SEQ ID NO: 27) | PEgRNA spacer (AG PAM) |
| 347 | TAGATCTTTCTTGTGATTATC (SEQ ID NO: 347) | PEgRNA spacer (AG PAM) |
| 348 | GTAGATCTTTCTTGTGATTATC (SEQ ID NO: 348) | PEgRNA spacer (AG PAM) |
| 349 | AATCA | PBS |
| 350 | AATCAC | PBS |
| 351 | AATCACA | PBS |
| 352 | AATCACAA | PBS |
| 353 | AATCACAAG | PBS |
| 354 | AATCACAAGA (SEQ ID NO: 354) | PBS |
| 355 | AATCACAAGAA (SEQ ID NO: 355) | PBS |
| 356 | AATCACAAGAAA (SEQ ID NO: 356) | PBS |
| 357 | AATCACAAGAAAG (SEQ ID NO: 357) | PBS |
| 358 | AATCACAAGAAAGA (SEQ ID NO: 358) | PBS |
| 359 | AATCACAAGAAAGAT (SEQ ID NO: 359) | PBS |
| 360 | AATCACAAGAAAGATC (SEQ ID NO: 360) | PBS |
| 361 | AATCACAAGAAAGATCT (SEQ ID NO: 361) | PBS |
| 362 | AATCACAAGAAAGATCTA (SEQ ID NO: 362) | PBS |
| 363 | AATCACAAGAAAGATCTAC (SEQ ID NO: 363) | PBS |
| 364 | CCCATACAGAAAGCTTCTGCATCCAAAACTGAT | RTT*2, 3, 4 |
| | (SEQ ID NO: 364) | |
| 365 | CCCATACAGAAAGCTTCTGCATCCAAAATTGAT | RTT*1, 2, 3, 4 (AG-to-AA PAM silencing |
| | (SEQ ID NO: 365) | edit) |
| 366 | TCCCATACAGAAAGCTTCTGCATCCAAAACTGAT | RTT*2, 3, 4 |
| | (SEQ ID NO: 366) | |
| 367 | TCCCATACAGAAAGCTTCTGCATCCAAAATTGAT | RTT*1, 2, 3, 4 (AG-to-AA PAM silencing |
| | (SEQ ID NO: 367) | edit) |
| 368 | ATCCCATACAGAAAGCTTCTGCATCCAAAACTGA | RTT*2, 3, 4 |
| | T (SEQ ID NO: 368) | |
| 369 | ATCCCATACAGAAAGCTTCTGCATCCAAAATTGA | RTT*1, 2, 3, 4 (AG-to-AA PAM silencing |
| | T (SEQ ID NO: 369) | edit) |
| 370 | GATCCCATACAGAAAGCTTCTGCATCCAAAACTG | RTT*2, 3, 4 |
| | AT (SEQ ID NO: 370) | |
| 371 | GATCCCATACAGAAAGCTTCTGCATCCAAAATTG | RTT*1, 2, 3, 4 (AG-to-AA PAM silencing |
| | AT (SEQ ID NO: 371) | edit) |
| 372 | TGATCCCATACAGAAAGCTTCTGCATCCAAAACT | RTT*2, 3, 4 |
| | GAT (SEQ ID NO: 372) | |
| 373 | TGATCCCATACAGAAAGCTTCTGCATCCAAAATT | RTT*1, 2, 3, 4 (AG-to-AA PAM silencing |
| | GAT (SEQ ID NO: 373) | edit) |
| 374 | CTGATCCCATACAGAAAGCTTCTGCATCCAAAAC | RTT*2, 3, 4 |
| | TGAT (SEQ ID NO: 374) | |
| 375 | CTGATCCCATACAGAAAGCTTCTGCATCCAAAAT | RTT*1, 2, 3, 4 (AG-to-AA PAM silencing |
| | TGAT (SEQ ID NO: 375) | edit) |
| 376 | CCTGATCCCATACAGAAAGCTTCTGCATCCAAAA | RTT*2, 3, 4 |
| | CTGAT (SEQ ID NO: 376) | |
| 377 | CCTGATCCCATACAGAAAGCTTCTGCATCCAAAA | RTT*1, 2, 3, 4 (AG-to-AA PAM silencing |
| | TTGAT (SEQ ID NO: 377) | edit) |
| 378 | GCCTGATCCCATACAGAAAGCTTCTGCATCCAAA | RTT*2, 3, 4 |
| | ACTGAT (SEQ ID NO: 378) | |
| 379 | GCCTGATCCCATACAGAAAGCTTCTGCATCCAAA | RTT*1, 2, 3, 4 (AG-to-AA PAM silencing |
| | ATTGAT (SEQ ID NO: 379) | edit) |
| 380 | AAAGCTTCTGCATCCAAAAC (SEQ ID NO: 380) | PE3 ngRNA spacer (TG PAM) |
| 381 | AAAGCTTCTGCATCCAAAAT (SEQ ID NO: 381) | PE3b*1 ngRNA spacer (TG PAM) |
| 4 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b*2 ngRNA spacer (AG PAM) |
| 195 | ACATCTTCCTTAGGAACTCC (SEQ ID NO: 195) | PE3 ngRNA spacer (TG PAM) |
| 197 | ACTCCTGGAACTGAGCCACG (SEQ ID NO: 197) | PE3 ngRNA spacer (TG PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 383 | GATCCCATACAGAAAGCTTC (SEQ ID NO: 383) | PE3b*3 ngRNA spacer (TG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b*4 ngRNA spacer (AG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |

TABLE 5

| Sequence | | |
|---|---|---|
| Number | Sequence | Description |
| 385 | AGATCTTTCTTGTGATT (SEQ ID NO: 385) | PEgRNA spacer (ATCAGT PAM) |
| 386 | TAGATCTTTCTTGTGATT (SEQ ID NO: 386) | PEgRNA spacer (ATCAGT PAM) |
| 387 | GTAGATCTTTCTTGTGATT (SEQ ID NO: 387) | PEgRNA spacer (ATCAGT PAM) |
| 128 | AGTAGATCTTTCTTGTGATT (SEQ ID NO: 128) | PEgRNA spacer (ATCAGT PAM) |
| 388 | CAGTAGATCTTTCTTGTGATT (SEQ ID NO: 388) | PEgRNA spacer (ATCAGT PAM) |
| 389 | TCAGTAGATCTTTCTTGTGATT (SEQ ID NO: 389) | PEgRNA spacer (ATCAGT PAM) |
| 390 | CACAA | PBS |
| 391 | CACAAG | PBS |
| 392 | CACAAGA | PBS |
| 393 | CACAAGAA | PBS |
| 394 | CACAAGAAA | PBS |
| 395 | CACAAGAAAG (SEQ ID NO: 395) | PBS |
| 396 | CACAAGAAAGA (SEQ ID NO: 396) | PBS |
| 397 | CACAAGAAAGAT (SEQ ID NO: 397) | PBS |
| 398 | CACAAGAAAGATC (SEQ ID NO: 398) | PBS |
| 399 | CACAAGAAAGATCT (SEQ ID NO: 399) | PBS |
| 400 | CACAAGAAAGATCTA (SEQ ID NO: 400) | PBS |
| 401 | CACAAGAAAGATCTAC (SEQ ID NO: 401) | PBS |
| 402 | CACAAGAAAGATCTACT (SEQ ID NO: 402) | PBS |
| 403 | CACAAGAAAGATCTACTG (SEQ ID NO: 403) | PBS |
| 404 | CACAAGAAAGATCTACTGA (SEQ ID NO: 404) | PBS |
| 405 | CCCATACAGAAAGCTTCTGCATCCAAAACTGATA | RTT |
| | AT (SEQ ID NO: 405) | |
| 406 | TCCCATACAGAAAGCTTCTGCATCCAAAACTGAT | RTT |
| | AAT (SEQ ID NO: 406) | |
| 407 | ATCCCATACAGAAAGCTTCTGCATCCAAAACTGA | RTT |
| | TAAT (SEQ ID NO: 407) | |
| 408 | GATCCCATACAGAAAGCTTCTGCATCCAAAACTG | RTT |
| | ATAAT (SEQ ID NO: 408) | |
| 409 | TGATCCCATACAGAAAGCTTCTGCATCCAAAACT | RTT |
| | GATAAT (SEQ ID NO: 409) | |
| 410 | AAGCTTCTGCATCCAAAACT (SEQ ID NO: 410) | PE3 ngRNA spacer (GATAAT PAM) |
| 64 | ACGTGAAGACAGGTGTCTTG (SEQ ID NO: 64) | PE3 ngRNA spacer (CTGGGT PAM) |
| 411 | AGAAAGCTTCTGCATCCAAA (SEQ ID NO: 411) | PE3 ngRNA spacer (ACTGAT PAM) |
| 412 | CAGGTGTCTTGCTGGGTTTC (SEQ ID NO: 412) | PE3 ngRNA spacer (CAAAGT PAM) |
| 413 | CTGGGTTTCCAAAGTGGAAG (SEQ ID NO: 413) | PE3 ngRNA spacer (CCTGAT PAM) |
| 414 | TGGAACTGAGCCACGTGAAG (SEQ ID NO: 414) | PE3 ngRNA spacer (ACAGGT PAM) |

TABLE 6

| Sequence | | |
|---|---|---|
| Number | Sequence | Description |
| 415 | ATCAGTTTTGGATGCAG (SEQ ID NO: 415) | PEgRNA spacer (AAG PAM) |
| 416 | TATCAGTTTTGGATGCAG (SEQ ID NO: 416) | PEgRNA spacer (AAG PAM) |
| 417 | TTATCAGTTTTGGATGCAG (SEQ ID NO: 417) | PEgRNA spacer (AAG PAM) |
| 418 | ATTATCAGTTTTGGATGCAG (SEQ ID NO: 418) | PEgRNA spacer (AAG PAM) |
| 419 | GATTATCAGTTTTGGATGCAG (SEQ ID NO: 419) | PEgRNA spacer (AAG PAM) |
| 420 | TGATTATCAGTTTTGGATGCAG (SEQ ID NO: 420) | PEgRNA spacer (AAG PAM) |
| 421 | CATCC | PBS |
| 422 | CATCCA | PBS |
| 423 | CATCCAA | PBS |
| 424 | CATCCAAA | PBS |
| 425 | CATCCAAAA | PBS |
| 426 | CATCCAAAAC (SEQ ID NO: 426) | PBS |
| 427 | CATCCAAAACT (SEQ ID NO: 427) | PBS |
| 428 | CATCCAAAACTG (SEQ ID NO: 428) | PBS |
| 429 | CATCCAAAACTGA (SEQ ID NO: 429) | PBS |
| 430 | CATCCAAAACTGAT (SEQ ID NO: 430) | PBS |
| 431 | CATCCAAAACTGATA (SEQ ID NO: 431) | PBS |
| 432 | CATCCAAAACTGATAA (SEQ ID NO: 432) | PBS |
| 433 | CATCCAAAACTGATAAT (SEQ ID NO: 433) | PBS |
| 434 | CATCCAAAACTGATAATC (SEQ ID NO: 434) | PBS |
| 435 | CATCCAAAACTGATAATCA (SEQ ID NO: 435) | PBS |
| 436 | CCCATACAGAAAGCTTCTG (SEQ ID NO: 436) | RTT |
| 437 | CCCATACAGAAAGTTTCTG (SEQ ID NO: 437) | RTT (AAG-to-AAA PAM silencing edit) |
| 438 | TCCCATACAGAAAGCTTCTG (SEQ ID NO: 438) | RTT |
| 439 | TCCCATACAGAAAGTTTCTG (SEQ ID NO: 439) | RTT (AAG-to-AAA PAM silencing edit) |
| 440 | ATCCCATACAGAAAGCTTCTG (SEQ ID NO: 440) | RTT |
| 441 | ATCCCATACAGAAAGTTTCTG (SEQ ID NO: 441) | RTT (AAG-to-AAA PAM silencing edit) |
| 442 | GATCCCATACAGAAAGCTTCTG (SEQ ID NO: 442) | RTT |
| 443 | GATCCCATACAGAAAGTTTCTG (SEQ ID NO: 443) | RTT (AAG-to-AAA PAM silencing edit) |
| 444 | TGATCCCATACAGAAAGCTTCTG (SEQ ID NO: | RTT |
| | 444) | |
| 445 | TGATCCCATACAGAAAGTTTCTG (SEQ ID NO: | RTT (AAG-to-AAA PAM silencing edit) |
| | 445) | |
| 446 | CTGATCCCATACAGAAAGCTTCTG (SEQ ID NO: | RTT |
| | 446) | |
| 447 | CTGATCCCATACAGAAAGTTTCTG (SEQ ID NO: | RTT (AAG-to-AAA PAM silencing edit) |
| | 447) | |
| 448 | CCTGATCCCATACAGAAAGCTTCTG (SEQ ID NO: | RTT |
| | 448) | |
| 449 | CCTGATCCCATACAGAAAGTTTCTG (SEQ ID NO: | RTT (AAG-to-AAA PAM silencing edit) |
| | 449) | |
| 450 | GCCTGATCCCATACAGAAAGCTTCTG (SEQ ID | RTT |
| | NO: 450) | |
| 451 | GCCTGATCCCATACAGAAAGTTTCTG (SEQ ID | RTT (AAG-to-AAA PAM silencing edit) |
| | NO: 451) | |
| 452 | AGCCTGATCCCATACAGAAAGCTTCTG (SEQ ID | RTT |
| | NO: 452) | |
| 453 | AGCCTGATCCCATACAGAAAGTTTCTG (SEQ ID | RTT (AAG-to-AAA PAM silencing edit) |
| | NO: 453) | |
| 454 | AAGCCTGATCCCATACAGAAAGCTTCTG (SEQ ID | RTT |
| | NO: 454) | |
| 455 | AAGCCTGATCCCATACAGAAAGTTTCTG (SEQ ID | RTT (AAG-to-AAA PAM silencing edit) |
| | NO: 455) | |
| 456 | GAAGCCTGATCCCATACAGAAAGCTTCTG (SEQ | RTT |
| | ID NO: 456) | |
| 457 | GAAGCCTGATCCCATACAGAAAGTTTCTG (SEQ | RTT (AAG-to-AAA PAM silencing edit) |
| | ID NO: 457) | |
| 458 | GGAAGCCTGATCCCATACAGAAAGCTTCTG (SEQ | RTT |
| | ID NO: 458) | |
| 459 | GGAAGCCTGATCCCATACAGAAAGTTTCTG (SEQ | RTT (AAG-to-AAA PAM silencing edit) |
| | ID NO: 459) | |
| 460 | TGGAAGCCTGATCCCATACAGAAAGCTTCTG | RTT |
| | (SEQ ID NO: 460) | |
| 461 | TGGAAGCCTGATCCCATACAGAAAGTTTCTG | RTT (AAG-to-AAA PAM silencing edit) |
| | (SEQ ID NO: 461) | |
| 462 | GTGGAAGCCTGATCCCATACAGAAAGCTTCTG | RTT |
| | (SEQ ID NO: 462) | |
| 463 | GTGGAAGCCTGATCCCATACAGAAAGTTTCTG | RTT (AAG-to-AAA PAM silencing edit) |
| | (SEQ ID NO: 463) | |
| 464 | AGTGGAAGCCTGATCCCATACAGAAAGCTTCTG | RTT |
| | (SEQ ID NO: 464) | |
| 465 | AGTGGAAGCCTGATCCCATACAGAAAGTTTCTG | RTT (AAG-to-AAA PAM silencing edit) |
| | (SEQ ID NO: 465) | |
| 466 | AAGTGGAAGCCTGATCCCATACAGAAAGCTTCT | RTT |
| | G (SEQ ID NO: 466) | |
| 467 | AAGTGGAAGCCTGATCCCATACAGAAAGTTTCTG | RTT (AAG-to-AAA PAM silencing edit) |
| | (SEQ ID NO: 467) | |
| 468 | AAAGTGGAAGCCTGATCCCATACAGAAAGCTTC | RTT |
| | TG (SEQ ID NO: 468) | |
| 469 | AAAGTGGAAGCCTGATCCCATACAGAAAGTTTCT | RTT (AAG-to-AAA PAM silencing edit) |
| | G (SEQ ID NO: 469) | |
| 470 | CAAAGTGGAAGCCTGATCCCATACAGAAAGCTT | RTT |
| | CTG (SEQ ID NO: 470) | |
| 471 | CAAAGTGGAAGCCTGATCCCATACAGAAAGTTT | RTT (AAG-to-AAA PAM silencing edit) |
| | CTG (SEQ ID NO: 471) | |
| 472 | CCAAAGTGGAAGCCTGATCCCATACAGAAAGCT | RTT |
| | TCTG (SEQ ID NO: 472) | |
| 473 | CCAAAGTGGAAGCCTGATCCCATACAGAAAGTT | RTT (AAG-to-AAA PAM silencing edit) |
| | TCTG (SEQ ID NO: 473) | |
| 474 | TCCAAAGTGGAAGCCTGATCCCATACAGAAAGC | RTT |
| | TTCTG (SEQ ID NO: 474) | |
| 475 | TCCAAAGTGGAAGCCTGATCCCATACAGAAAGT | RTT (AAG-to-AAA PAM silencing edit) |
| | TTCTG (SEQ ID NO: 475) | |
| 476 | TTCCAAAGTGGAAGCCTGATCCCATACAGAAAG | RTT |
| | CTTCTG (SEQ ID NO: 476) | |
| 477 | TTCCAAAGTGGAAGCCTGATCCCATACAGAAAG | RTT (AAG-to-AAA PAM silencing edit) |
| | TTTCTG (SEQ ID NO: 477) | |
| 478 | TTTCCAAAGTGGAAGCCTGATCCCATACAGAAA | RTT |
| | GCTTCTG (SEQ ID NO: 478) | |
| 479 | TTTCCAAAGTGGAAGCCTGATCCCATACAGAAA | RTT (AAG-to-AAA PAM silencing edit) |
| | GTTTCTG (SEQ ID NO: 479) | |
| 480 | AAGGCTTCATACATCTTCCT (SEQ ID NO: 480) | PE3 ngRNA spacer (TAG PAM) |
| 481 | AGTGGAAGCCTGATCCCATA (SEQ ID NO: 481) | PE3b ngRNA spacer (CAG PAM) |
| 482 | CACTACTTACCATCTCTTTC (SEQ ID NO: 482) | PE3 ngRNA spacer (AAG PAM) |
| 483 | CCTTAGGAACTCCTGGAACT (SEQ ID NO: 483) | PE3 ngRNA spacer (GAG PAM) |
| 484 | GAAGCCTGATCCCATACAGA (SEQ ID NO: 484) | PE3b ngRNA spacer (AAG PAM) |
| 485 | GGAACTGAGCCACGTGAAGA (SEQ ID NO: 485) | PE3 ngRNA spacer (CAG PAM) |
| 486 | GGTGTCTTGCTGGGTTTCCA (SEQ ID NO: 486) | PE3 ngRNA spacer (AAG PAM) |
| 487 | TCCTGGAACTGAGCCACGTG (SEQ ID NO: 487) | PE3 ngRNA spacer (AAG PAM) |

| | | |
|---|---|---|
| 488 | TTGCTGGGTTTCCAAAGTGG (SEQ ID NO: 488) | PE3 ngRNA spacer (AAG PAM) |

TABLE 7

| Sequence | | |
|---|---|---|
| Number | Sequence | Description |
| 489 | AAGCTTTCTGTATGGAT (SEQ ID NO: 489) | PEgRNA spacer (CAG; CAGG PAM) |
| 490 | GAAGCTTTCTGTATGGAT (SEQ ID NO: 490) | PEgRNA spacer (CAG; CAGG PAM) |
| 491 | AGAAGCTTTCTGTATGGAT (SEQ ID NO: 491) | PEgRNA spacer (CAG; CAGG PAM) |
| 492 | CAGAAGCTTTCTGTATGGAT (SEQ ID NO: 492) | PEgRNA spacer (CAG; CAGG PAM) |
| 493 | GCAGAAGCTTTCTGTATGGAT (SEQ ID NO: 493) | PEgRNA spacer (CAG; CAGG PAM) |
| 494 | TGCAGAAGCTTTTGTATGGAT (SEQ ID NO: 494) | PEgRNA spacer (CAG; CAGG PAM) |
| 495 | CATAC | PBS |
| 496 | CATACA | PBS |
| 497 | CATACAG | PBS |
| 498 | CATACAGA | PBS |
| 499 | CATACAGAA | PBS |
| 500 | CATACAGAAA (SEQ ID NO: 500) | PBS |
| 501 | CATACAGAAAG (SEQ ID NO: 501) | PBS |
| 502 | CATACAGAAAGC (SEQ ID NO: 502) | PBS |
| 503 | CATACAGAAAGCT (SEQ ID NO: 503) | PBS |
| 504 | CATACAGAAAGCTT (SEQ ID NO: 504) | PBS |
| 505 | CATACAGAAAGCTTC (SEQ ID NO: 505) | PBS |
| 506 | CATACAGAAAGCTTCT (SEQ ID NO: 506) | PBS |
| 507 | CATACAGAAAGCTTCTG (SEQ ID NO: 507) | PBS |
| 508 | CATACAGAAAGCTTCTGC (SEQ ID NO: 508) | PBS |
| 509 | CATACAGAAAGCTTCTGCA (SEQ ID NO: 509) | PBS |
| 510 | AGCCTGATCC (SEQ ID NO: 510) | RTT |
| 511 | AAGCCTGATCC (SEQ ID NO: 511) | RTT |
| 512 | GAAGCCTGATCC (SEQ ID NO: 512) | RTT |
| 513 | GGAAGCCTGATCC (SEQ ID NO: 513) | RTT |
| 514 | TGGAAGCCTGATCC (SEQ ID NO: 514) | RTT |
| 515 | GTGGAAGCCTGATCC (SEQ ID NO: 515) | RTT |
| 516 | AGTGGAAGCCTGATCC (SEQ ID NO: 516) | RTT |
| 517 | AAGTGGAAGCCTGATCC (SEQ ID NO: 517) | RTT |
| 518 | AAAGTGGAAGCCTGATCC (SEQ ID NO: 518) | RTT |
| 519 | CAAAGTGGAAGCCTGATCC (SEQ ID NO: 519) | RTT |
| 520 | CCAAAGTGGAAGCCTGATCC (SEQ ID NO: 520) | RTT |
| 521 | TCCAAAGTGGAAGCCTGATCC (SEQ ID NO: 521) | RTT |
| 522 | TTCCAAAGTGGAAGCCTGATCC (SEQ ID NO: 522) | RTT |
| 523 | TTTCCAAAGTGGAAGCCTGATCC (SEQ ID NO: | RTT |
| | 523) | |
| 524 | GTTTCCAAAGTGGAAGCCTGATCC (SEQ ID NO: | RTT |
| | 524) | |
| 525 | GGTTTCCAAAGTGGAAGCCTGATCC (SEQ ID NO: | RTT |
| | 525) | |
| 526 | GGGTTTCCAAAGTGGAAGCCTGATCC (SEQ ID | RTT |
| | NO: 526) | |
| 527 | TGGGTTTCCAAAGTGGAAGCCTGATCC (SEQ ID | RTT |
| | NO: 527) | |
| 528 | CTGGGTTTCCAAAGTGGAAGCCTGATCC (SEQ ID | RTT |
| | NO: 528) | |
| 529 | GCTGGGTTTCCAAAGTGGAAGCCTGATCC (SEQ | RTT |
| | ID NO: 529) | |
| 530 | TGCTGGGTTTCCAAAGTGGAAGCCTGATCC (SEQ | RTT |
| | ID NO: 530) | |
| 531 | TTGCTGGGTTTCCAAAGTGGAAGCCTGATCC | RTT |
| | (SEQ ID NO: 531) | |
| 532 | CTTGCTGGGTTTCCAAAGTGGAAGCCTGATCC | RTT |
| | (SEQ ID NO: 532) | |
| 533 | TCTTGCTGGGTTTCCAAAGTGGAAGCCTGATCC | RTT |
| | (SEQ ID NO: 533) | |
| 534 | GTCTTGCTGGGTTTCCAAAGTGGAAGCCTGATCC | RTT |
| | (SEQ ID NO: 534) | |
| 535 | TGTCTTGCTGGGTTTCCAAAGTGGAAGCCTGATC | RTT |
| | C (SEQ ID NO: 535) | |
| 536 | GTGTCTTGCTGGGTTTCCAAAGTGGAAGCCTGAT | RTT |
| | CC (SEQ ID NO: 536) | |
| 537 | GGTGTCTTGCTGGGTTTCCAAAGTGGAAGCCTGA | RTT |
| | TCC (SEQ ID NO: 537) | |
| 538 | AGGTGTCTTGCTGGGTTTCCAAAGTGGAAGCCTG | RTT |
| | ATCC (SEQ ID NO: 538) | |
| 539 | CAGGTGTCTTGCTGGGTTTCCAAAGTGGAAGCCT | RTT |
| | GATCC (SEQ ID NO: 539) | |
| 540 | ACAGGTGTCTTGCTGGGTTTCCAAAGTGGAAGCC | RTT |
| | TGATCC (SEQ ID NO: 540) | |
| 480 | AAGGCTTCATACATCTTCCT (SEQ ID NO: 480) | PE3 ngRNA spacer (TAG; TAGG PAM) |
| 64 | ACGTGAAGACAGGTGTCTTG (SEQ ID NO: 64) | PE3 ngRNA spacer (CTGG PAM) |
| 482 | CACTACTTACCATCTCTTTC (SEQ ID NO: 482) | PE3 ngRNA spacer (AAG; AAGG PAM) |
| 541 | CAGAAAATAATTTCATTATT (SEQ ID NO: 541) | PE3 ngRNA spacer (CTGG PAM) |
| 483 | CCTTAGGAACTCCTGGAACT (SEQ ID NO: 483) | PE3 ngRNA spacer (GAG PAM) |
| 203 | CGTGAAGACAGGTGTCTTGC (SEQ ID NO: 203) | PE3 ngRNA spacer (TGGG PAM) |
| 485 | GGAACTGAGCCACGTGAAGA (SEQ ID NO: 485) | PE3 ngRNA spacer (CAG; CAGG PAM) |
| 486 | GGTGTCTTGCTGGGTTTCCA (SEQ ID NO: 486) | PE3 ngRNA spacer (AAG PAM) |
| 542 | TACATCTTCCTTAGGAACTC (SEQ ID NO: 542) | PE3 ngRNA spacer (CTGG PAM) |
| 487 | TCCTGGAACTGAGCCACGTG (SEQ ID NO: 487) | PE3 ngRNA spacer (AAG PAM) |
| 543 | TGTCTTGCTGGGTTTCCAAA (SEQ ID NO: 543) | PE3 ngRNA spacer (GTGG PAM) |
| 488 | TTGCTGGGTTTCCAAAGTGG (SEQ ID NO: 488) | PE3 ngRNA spacer (AAG PAM) |
| 325 | CGTGAAGACAGGTGTCTTGCGTTTTAGAGCTAGA | PE3 ngRNA (TGGG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 333 | GCGTGAAGACAGGTGTCTTGCGTTTTAGAGCTAG | PE3 ngRNA (TGGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 340 | GCGTGAAGACAGGTGTCTTGCGTTTTAGAGCTAG | PE3 ngRNA (TGGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |

TABLE 8

| Sequence | | |
|---|---|---|
| Number | Sequence | Description |
| 544 | GTGTCTTGCTGGGTTTC (SEQ ID NO: 544) | PEgRNA spacer (CAAAGT PAM) |
| 545 | GGTGTCTTGCTGGGTTTC (SEQ ID NO: 545) | PEgRNA spacer (CAAAGT PAM) |
| 546 | AGGTGTCTTGCTGGGTTTC (SEQ ID NO: 546) | PEgRNA spacer (CAAAGT PAM) |
| 412 | CAGGTGTCTTGCTGGGTTTC (SEQ ID NO: 412) | PEgRNA spacer (CAAAGT PAM) |
| 547 | ACAGGTGTCTTGCTGGGTTTC (SEQ ID NO: 547) | PEgRNA spacer (CAAAGT PAM) |
| 548 | GACAGGTGTCTTGCTGGGTTTC (SEQ ID NO: 548) | PEgRNA spacer (CAAAGT PAM) |
| 549 | ACCCA | PBS |
| 550 | ACCCAG | PBS |
| 551 | ACCCAGC | PBS |
| 552 | ACCCAGCA | PBS |
| 553 | ACCCAGCAA | PBS |
| 554 | ACCCAGCAAG (SEQ ID NO: 554) | PBS |
| 555 | ACCCAGCAAGA (SEQ ID NO: 555) | PBS |
| 556 | ACCCAGCAAGAC (SEQ ID NO: 556) | PBS |
| 557 | ACCCAGCAAGACA (SEQ ID NO: 557) | PBS |
| 558 | ACCCAGCAAGACAC (SEQ ID NO: 558) | PBS |
| 559 | ACCCAGCAAGACACC (SEQ ID NO: 559) | PBS |
| 560 | ACCCAGCAAGACACCT (SEQ ID NO: 560) | PBS |
| 561 | ACCCAGCAAGACACCTG (SEQ ID NO: 561) | PBS |
| 562 | ACCCAGCAAGACACCTGT (SEQ ID NO: 562) | PBS |
| 563 | ACCCAGCAAGACACCTGTC (SEQ ID NO: 563) | PBS |
| 564 | GATCAGGCTTCCACATTGGAA (SEQ ID NO: 564) | RTT (CAAAGT-to-CAATGT PAM silencing |
| | | edit) |
| 565 | GATCAGGCTTCCACGTTGGAA (SEQ ID NO: 565) | RTT (CAAAGT-to-CAACGT PAM silencing |
| | | edit) |
| 566 | GATCAGGCTTCCACTTTGGAA (SEQ ID NO: 566) | RTT |
| 567 | GGATCAGGCTTCCACATTGGAA (SEQ ID NO: 567) | RTT (CAAAGT-to-CAATGT PAM silencing |
| | | edit) |
| 568 | GGATCAGGCTTCCACGTTGGAA (SEQ ID NO: 568) | RTT (CAAAGT-to-CAACGT PAM silencing |
| | | edit) |
| 569 | GGATCAGGCTTCCACTTTGGAA (SEQ ID NO: 569) | RTT |
| 570 | GGGATCAGGCTTCCACATTGGAA (SEQ ID NO: | RTT (CAAAGT-to-CAATGT PAM silencing |
| | 570) | edit) |
| 571 | GGGATCAGGCTTCCACGTTGGAA (SEQ ID NO: | RTT (CAAAGT-to-CAACGT PAM silencing |
| | 571) | edit) |
| 572 | GGGATCAGGCTTCCACTTTGGAA (SEQ ID NO: | RTT |
| | 572) | |
| 573 | TGGGATCAGGCTTCCACATTGGAA (SEQ ID NO: | RTT (CAAAGT-to-CAATGT PAM silencing |
| | 573) | edit) |
| 574 | TGGGATCAGGCTTCCACGTTGGAA (SEQ ID NO: | RTT (CAAAGT-to-CAACGT PAM silencing |
| | 574) | edit) |
| 575 | TGGGATCAGGCTTCCACTTTGGAA (SEQ ID NO: | RTT |
| | 575) | |
| 576 | ATGGGATCAGGCTTCCACATTGGAA (SEQ ID NO: | RTT (CAAAGT-to-CAATGT PAM silencing |
| | 576) | edit) |
| 577 | ATGGGATCAGGCTTCCACGTTGGAA (SEQ ID NO: | RTT (CAAAGT-to-CAACGT PAM silencing |
| | 577) | edit) |
| 578 | ATGGGATCAGGCTTCCACTTTGGAA (SEQ ID NO: | RTT |
| | 578) | |
| 579 | TATGGGATCAGGCTTCCACATTGGAA (SEQ ID | RTT (CAAAGT-to-CAATGT PAM silencing |
| | NO: 579) | edit) |
| 580 | TATGGGATCAGGCTTCCACGTTGGAA (SEQ ID | RTT (CAAAGT-to-CAACGT PAM silencing |
| | NO: 580) | edit) |
| 581 | TATGGGATCAGGCTTCCACTTTGGAA (SEQ ID | RTT |
| | NO: 581) | |
| 582 | GTATGGGATCAGGCTTCCACATTGGAA (SEQ ID | RTT (CAAAGT-to-CAATGT PAM silencing |
| | NO: 582) | edit) |
| 583 | GTATGGGATCAGGCTTCCACGTTGGAA (SEQ ID | RTT (CAAAGT-to-CAACGT PAM silencing |
| | NO: 583) | edit) |
| 584 | GTATGGGATCAGGCTTCCACTTTGGAA (SEQ ID | RTT |
| | NO: 584) | |
| 585 | TGTATGGGATCAGGCTTCCACATTGGAA (SEQ ID | RTT (CAAAGT-to-CAATGT PAM silencing |
| | NO: 585) | edit) |
| 586 | TGTATGGGATCAGGCTTCCACGTTGGAA (SEQ ID | RTT (CAAAGT-to-CAACGT PAM silencing |
| | NO: 586) | edit) |
| 587 | TGTATGGGATCAGGCTTCCACTTTGGAA (SEQ ID | RTT |
| | NO: 587) | |
| 588 | CTGTATGGGATCAGGCTTCCACATTGGAA (SEQ | RTT (CAAAGT-to-CAATGT PAM silencing |
| | ID NO: 588) | edit) |
| 589 | CTGTATGGGATCAGGCTTCCACGTTGGAA (SEQ | RTT (CAAAGT-to-CAACGT PAM silencing |
| | ID NO: 589) | edit) |
| 590 | CTGTATGGGATCAGGCTTCCACTTTGGAA (SEQ | RTT |
| | ID NO: 590) | |
| 591 | TCTGTATGGGATCAGGCTTCCACATTGGAA (SEQ | RTT (CAAAGT-to-CAATGT PAM silencing |
| | ID NO: 591) | edit) |
| 592 | TCTGTATGGGATCAGGCTTCCACGTTGGAA (SEQ | RTT (CAAAGT-to-CAACGT PAM silencing |
| | ID NO: 592) | edit) |
| 593 | TCTGTATGGGATCAGGCTTCCACTTTGGAA (SEQ | RTT |
| | ID NO: 593) | |
| 594 | TTCTGTATGGGATCAGGCTTCCACATTGGAA | RTT (CAAAGT-to-CAATGT PAM silencing |
| | (SEQ ID NO: 594) | edit) |
| 595 | TTCTGTATGGGATCAGGCTTCCACGTTGGAA | RTT (CAAAGT-to-CAACGT PAM silencing |
| | (SEQ ID NO: 595) | edit) |
| 596 | TTCTGTATGGGATCAGGCTTCCACTTTGGAA | RTT |
| | (SEQ ID NO: 596) | |
| 597 | TTTCTGTATGGGATCAGGCTTCCACATTGGAA | RTT (CAAAGT-to-CAATGT PAM silencing |
| | (SEQ ID NO: 597) | edit) |
| 598 | TTTCTGTATGGGATCAGGCTTCCACGTTGGAA | RTT (CAAAGT-to-CAACGT PAM silencing |
| | (SEQ ID NO: 598) | edit) |
| 599 | TTTCTGTATGGGATCAGGCTTCCACTTTGGAA | RTT |
| | (SEQ ID NO: 599) | |
| 600 | CTTTCTGTATGGGATCAGGCTTCCACATTGGAA | RTT (CAAAGT-to-CAATGT PAM silencing |
| | (SEQ ID NO: 600) | edit) |
| 601 | CTTTCTGTATGGGATCAGGCTTCCACGTTGGAA | RTT (CAAAGT-to-CAACGT PAM silencing |
| | (SEQ ID NO: 601) | edit) |
| 602 | CTTTCTGTATGGGATCAGGCTTCCACTTTGGAA | RTT |
| | (SEQ ID NO: 602) | |
| 603 | GCTTTCTGTATGGGATCAGGCTTCCACATTGGAA | RTT (CAAAGT-to-CAATGT PAM silencing |
| | (SEQ ID NO: 603) | edit) |
| 604 | GCTTTCTGTATGGGATCAGGCTTCCACGTTGGAA | RTT (CAAAGT-to-CAACGT PAM silencing |
| | (SEQ ID NO: 604) | edit) |
| 605 | GCTTTCTGTATGGGATCAGGCTTCCACTTTGGAA | RTT |
| | (SEQ ID NO: 605) | |
| 606 | AGCTTTCTGTATGGGATCAGGCTTCCACATTGGA | RTT (CAAAGT-to-CAATGT PAM silencing |
| | A (SEQ ID NO: 606) | edit) |
| 607 | AGCTTTCTGTATGGGATCAGGCTTCCACGTTGGA | RTT (CAAAGT-to-CAACGT PAM silencing |
| | A (SEQ ID NO: 607) | edit) |
| 608 | AGCTTTCTGTATGGGATCAGGCTTCCACTTTGGA | RTT |
| | A (SEQ ID NO: 608) | |
| 609 | AAGCTTTCTGTATGGGATCAGGCTTCCACATTGG | RTT (CAAAGT-to-CAATGT PAM silencing |
| | AA (SEQ ID NO: 609) | edit) |
| 610 | AAGCTTTCTGTATGGGATCAGGCTTCCACGTTGG | RTT (CAAAGT-to-CAACGT PAM silencing |
| | AA (SEQ ID NO: 610) | edit) |
| 611 | AAGCTTTCTGTATGGGATCAGGCTTCCACTTTGG | RTT |
| | AA (SEQ ID NO: 611) | |
| 612 | GAAGCTTTCTGTATGGGATCAGGCTTCCACATTG | RTT (CAAAGT-to-CAATGT PAM silencing |
| | GAA (SEQ ID NO: 612) | edit) |
| 613 | GAAGCTTTCTGTATGGGATCAGGCTTCCACGTTG | RTT (CAAAGT-to-CAACGT PAM silencing |
| | GAA (SEQ ID NO: 613) | edit) |
| 614 | GAAGCTTTCTGTATGGGATCAGGCTTCCACTTTG | RTT |
| | GAA (SEQ ID NO: 614) | |
| 615 | AGAAGCTTTCTGTATGGGATCAGGCTTCCACATT | RTT (CAAAGT-to-CAATGT PAM silencing |
| | GGAA (SEQ ID NO: 615) | edit) |
| 616 | AGAAGCTTTCTGTATGGGATCAGGCTTCCACGTT | RTT (CAAAGT-to-CAACGT PAM silencing |
| | GGAA (SEQ ID NO: 616) | edit) |
| 617 | AGAAGCTTTCTGTATGGGATCAGGCTTCCACTTT | RTT |
| | GGAA (SEQ ID NO: 617) | |
| 618 | CAGAAGCTTTCTGTATGGGATCAGGCTTCCACAT | RTT (CAAAGT-to-CAATGT PAM silencing |
| | TGGAA (SEQ ID NO: 618) | edit) |
| 619 | CAGAAGCTTTCTGTATGGGATCAGGCTTCCACGT | RTT (CAAAGT-to-CAACGT PAM silencing |
| | TGGAA (SEQ ID NO: 619) | edit) |
| 620 | CAGAAGCTTTCTGTATGGGATCAGGCTTCCACTT | RTT |
| | TGGAA (SEQ ID NO: 620) | |
| 621 | GCAGAAGCTTTCTGTATGGGATCAGGCTTCCACA | RTT (CAAAGT-to-CAATGT PAM silencing |
| | TTGGAA (SEQ ID NO: 621) | edit) |
| 622 | GCAGAAGCTTTCTGTATGGGATCAGGCTTCCACG | RTT (CAAAGT-to-CAACGT PAM silencing |
| | TTGGAA (SEQ ID NO: 622) | edit) |
| 623 | GCAGAAGCTTTCTGTATGGGATCAGGCTTCCACT | RTT |
| | TTGGAA (SEQ ID NO: 623) | |
| 25 | AAGATTCAGTAGATCTTTCT (SEQ ID NO: 25) | PE3 ngRNA spacer (TGTGAT PAM) |
| 128 | AGTAGATCTTTCTTGTGATT (SEQ ID NO: 128) | PE3 ngRNA spacer (ATCAGT PAM) |
| 129 | CATCACCTTTTCGCTTTTTC (SEQ ID NO: 129) | PE3 ngRNA spacer (CAAGAT PAM) |
| 130 | CCAAGATGGCTCAAGATTCA (SEQ ID NO: 130) | PE3 ngRNA spacer (GTAGAT PAM) |
| 131 | CTTTCTTGTGATTATCAGTT (SEQ ID NO: 131) | PE3 ngRNA spacer (TTGGAT PAM) |
| 133 | TCGCTTTTTCCAAGATGGCT (SEQ ID NO: 133) | PE3 ngRNA spacer (CAAGAT PAM) |
| 134 | TGGATGCAGAAGCTTTCTGT (SEQ ID NO: 134) | PE3 ngRNA spacer (ATGGAT PAM) |
| 135 | TTTTCCAAGATGGCTCAAGA (SEQ ID NO: 135) | PE3 ngRNA spacer (TTCAGT PAM) |

TABLE 9

| Sequence | | |
|---|---|---|
| Number | Sequence | Description |
| 624 | CGTGAAGACAGGTGTCT (SEQ ID NO: 624) | PEgRNA spacer (TG PAM) |
| 625 | ACGTGAAGACAGGTGTCT (SEQ ID NO: 625) | PEgRNA spacer (TG PAM) |
| 626 | CACGTGAAGACAGGTGTCT (SEQ ID NO: 626) | PEgRNA spacer (TG PAM) |
| 201 | CCACGTGAAGACAGGTGTCT (SEQ ID NO: 201) | PEgRNA spacer (TG PAM) |
| 627 | GCCACGTGAAGACAGGTGTCT (SEQ ID NO: 627) | PEgRNA spacer (TG PAM) |
| 628 | AGCCACGTGAAGACAGGTGTCT (SEQ ID NO: 628) | PEgRNA spacer (TG PAM) |
| 629 | CACCT | PBS |
| 630 | CACCTG | PBS |
| 631 | CACCTGT | PBS |
| 632 | CACCTGTC | PBS |
| 633 | CACCTGTCT | PBS |
| 634 | CACCTGTCTT (SEQ ID NO: 634) | PBS |
| 635 | CACCTGTCTTC (SEQ ID NO: 635) | PBS |
| 636 | CACCTGTCTTCA (SEQ ID NO: 636) | PBS |
| 637 | CACCTGTCTTCAC (SEQ ID NO: 637) | PBS |
| 638 | CACCTGTCTTCACG (SEQ ID NO: 638) | PBS |
| 639 | CACCTGTCTTCACGT (SEQ ID NO: 639) | PBS |
| 640 | CACCTGTCTTCACGTG (SEQ ID NO: 640) | PBS |
| 641 | CACCTGTCTTCACGTGG (SEQ ID NO: 641) | PBS |
| 642 | CACCTGTCTTCACGTGGC (SEQ ID NO: 642) | PBS |
| 643 | CACCTGTCTTCACGTGGCT (SEQ ID NO: 643) | PBS |
| 644 | GATCAGGCTTCCACTTTGGAAACCCAGCAAGA | RTT |
| | (SEQ ID NO: 644) | |
| 645 | GGATCAGGCTTCCACTTTGGAAACCCAGCAAGA | RTT |
| | (SEQ ID NO: 645) | |
| 646 | GGGATCAGGCTTCCACTTTGGAAACCCAGCAAG | RTT |
| | A (SEQ ID NO: 646) | |
| 647 | TGGGATCAGGCTTCCACTTTGGAAACCCAGCAA | RTT |
| | GA (SEQ ID NO: 647) | |
| 648 | ATGGGATCAGGCTTCCACTTTGGAAACCCAGCA | RTT |
| | AGA (SEQ ID NO: 648) | |
| 649 | TATGGGATCAGGCTTCCACTTTGGAAACCCAGCA | RTT |
| | AGA (SEQ ID NO: 649) | |
| 650 | GTATGGGATCAGGCTTCCACTTTGGAAACCCAGC | RTT |
| | AAGA (SEQ ID NO: 650) | |
| 651 | TGTATGGGATCAGGCTTCCACTTTGGAAACCCAG | RTT |
| | CAAGA (SEQ ID NO: 651) | |
| 652 | CTGTATGGGATCAGGCTTCCACTTTGGAAACCCA | RTT |
| | GCAAGA (SEQ ID NO: 652) | |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b ngRNA spacer (GG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b ngRNA spacer (TG PAM) |
| 29 | CCTTTTCGCTTTTTCCAAGA (SEQ ID NO: 29) | PE3 ngRNA spacer (TG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b ngRNA spacer (AG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG PAM) |
| 36 | GGCTTCCACTTTGGAAACCC (SEQ ID NO: 36) | PE3 ngRNA spacer (AG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 40 | TCCACTTTGGAAACCCAGCA (SEQ ID NO: 40) | PE3 ngRNA spacer (AG PAM) |
| 41 | TGATTATCAGTTTTGGATGC (SEQ ID NO: 41) | PE3 ngRNA spacer (AG PAM) |
| 43 | TGGGATCAGGCTTCCACTTT (SEQ ID NO: 43) | PE3b ngRNA spacer (GG PAM) |
| 44 | TTATCAGTTTTGGATGCAGA (SEQ ID NO: 44) | PE3 ngRNA spacer (AG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 60) | |

TABLE 10

| Sequence | | |
|---|---|---|
| Number | Sequence | Description |
| 653 | GAAGACAGGTGTCTTGC (SEQ ID NO: 653) | PEgRNA spacer (TG; TGG; TGGG PAM) |
| 654 | TGAAGACAGGTGTCTTGC (SEQ ID NO: 654) | PEgRNA spacer (TG; TGG; TGGG PAM) |
| 655 | GTGAAGACAGGTGTCTTGC (SEQ ID NO: 655) | PEgRNA spacer (TG; TGG; TGGG PAM) |
| 203 | CGTGAAGACAGGTGTCTTGC (SEQ ID NO: 203) | PEgRNA spacer (TG; TGG; TGGG PAM) |
| 656 | ACGTGAAGACAGGTGTCTTGC (SEQ ID NO: 656) | PEgRNA spacer (TG; TGG; TGGG PAM) |
| 657 | CACGTGAAGACAGGTGTCTTGC (SEQ ID NO: 657) | PEgRNA spacer (TG; TGG; TGGG PAM) |
| 658 | AGACA | PBS |
| 659 | AGACAC | PBS |
| 660 | AGACACC | PBS |
| 661 | AGACACCT | PBS |
| 662 | AGACACCTG | PBS |
| 663 | AGACACCTGT (SEQ ID NO: 663) | PBS |
| 664 | AGACACCTGTC (SEQ ID NO: 664) | PBS |
| 665 | AGACACCTGTCT (SEQ ID NO: 665) | PBS |
| 666 | AGACACCTGTCTT (SEQ ID NO: 666) | PBS |
| 667 | AGACACCTGTCTTC (SEQ ID NO: 667) | PBS |
| 668 | AGACACCTGTCTTCA (SEQ ID NO: 668) | PBS |
| 669 | AGACACCTGTCTTCAC (SEQ ID NO: 669) | PBS |
| 670 | AGACACCTGTCTTCACG (SEQ ID NO: 670) | PBS |
| 671 | AGACACCTGTCTTCACGT (SEQ ID NO: 671) | PBS |
| 672 | AGACACCTGTCTTCACGTG (SEQ ID NO: 672) | PBS |
| 673 | GATCAGGCTTCCACTTTGGAAACACAGCA (SEQ | RTT*2, 3, 4, 5 (TGG-to-TGT; TGGG-to-TGTG |
| | ID NO: 673) | PAM silencing edit) |
| 674 | GATCAGGCTTCCACTTTGGAAACCCAGCA (SEQ | RTT*1, 2, 3, 4, 5, 6 |
| | ID NO: 674) | |
| 675 | GATCAGGCTTCCACTTTGGAAACGCAGCA (SEQ | RTT*2, 3, 4, 5 (TGG-to-TGC; TGGG-to-TGCG |
| | ID NO: 675) | PAM silencing edit) |
| 676 | GATCAGGCTTCCACTTTGGAAACTCAGCA (SEQ | RTT*2, 3, 4, 5 (TGG-to-TGA; TGGG-to-TGAG |
| | ID NO: 676) | PAM silencing edit) |
| 677 | GGATCAGGCTTCCACTTTGGAAACACAGCA (SEQ | RTT*2, 3, 4, 5 (TGG-to-TGT; TGGG-to-TGTG |
| | ID NO: 677) | PAM silencing edit) |
| 678 | GGATCAGGCTTCCACTTTGGAAACCCAGCA (SEQ | RTT*1, 2, 3, 4, 5, 6 |
| | ID NO: 678) | |
| 679 | GGATCAGGCTTCCACTTTGGAAACGCAGCA (SEQ | RTT*2, 3, 4, 5 (TGG-to-TGC; TGGG-to-TGCG |
| | ID NO: 679) | PAM silencing edit) |
| 680 | GGATCAGGCTTCCACTTTGGAAACTCAGCA (SEQ | RTT*2, 3, 4, 5 (TGG-to-TGA; TGGG-to-TGAG |
| | ID NO: 680) | PAM silencing edit) |
| 681 | GGGATCAGGCTTCCACTTTGGAAACACAGCA | RTT*2, 3, 4, 5 (TGG-to-TGT; TGGG-to-TGTG |
| | (SEQ ID NO: 681) | PAM silencing edit) |
| 682 | GGGATCAGGCTTCCACTTTGGAAACCCAGCA | RTT*1, 2, 3, 4, 5, 6 |
| | (SEQ ID NO: 682) | |
| 683 | GGGATCAGGCTTCCACTTTGGAAACGCAGCA | RTT*2, 3, 4, 5 (TGG-to-TGC; TGGG-to-TGCG |
| | (SEQ ID NO: 683) | PAM silencing edit) |
| 684 | GGGATCAGGCTTCCACTTTGGAAACTCAGCA | RTT*2, 3, 4, 5 (TGG-to-TGA; TGGG-to-TGAG |
| | (SEQ ID NO: 684) | PAM silencing edit) |
| 685 | TGGGATCAGGCTTCCACTTTGGAAACACAGCA | RTT*2, 3, 4, 5 (TGG-to-TGT; TGGG-to-TGTG |
| | (SEQ ID NO: 685) | PAM silencing edit) |
| 686 | TGGGATCAGGCTTCCACTTTGGAAACCCAGCA | RTT*1, 2, 3, 4, 5, 6 |
| | (SEQ ID NO: 686) | |
| 687 | TGGGATCAGGCTTCCACTTTGGAAACGCAGCA | RTT*2, 3, 4, 5 (TGG-to-TGC; TGGG-to-TGCG |
| | (SEQ ID NO: 687) | PAM silencing edit) |
| 688 | TGGGATCAGGCTTCCACTTTGGAAACTCAGCA | RTT*2, 3, 4, 5 (TGG-to-TGA; TGGG-to-TGAG |
| | (SEQ ID NO: 688) | PAM silencing edit) |
| 689 | ATGGGATCAGGCTTCCACTTTGGAAACACAGCA | RTT*2, 3, 4, 5 (TGG-to-TGT; TGGG-to-TGTG |
| | (SEQ ID NO: 689) | PAM silencing edit) |
| 690 | ATGGGATCAGGCTTCCACTTTGGAAACCCAGCA | RTT*1, 2, 3, 4, 5, 6 |
| | (SEQ ID NO: 690) | |
| 691 | ATGGGATCAGGCTTCCACTTTGGAAACGCAGCA | RTT*2, 3, 4, 5 (TGG-to-TGC; TGGG-to-TGCG |
| | (SEQ ID NO: 691) | PAM silencing edit) |
| 692 | ATGGGATCAGGCTTCCACTTTGGAAACTCAGCA | RTT*2, 3, 4, 5 (TGG-to-TGA; TGGG-to-TGAG |
| | (SEQ ID NO: 692) | PAM silencing edit) |
| 693 | TATGGGATCAGGCTTCCACTTTGGAAACACAGCA | RTT*2, 3, 4, 5 (TGG-to-TGT; TGGG-to-TGTG |
| | (SEQ ID NO: 693) | PAM silencing edit) |
| 694 | TATGGGATCAGGCTTCCACTTTGGAAACCCAGCA | RTT*1, 2, 3, 4, 5, 6 |
| | (SEQ ID NO: 694) | |
| 695 | TATGGGATCAGGCTTCCACTTTGGAAACGCAGCA | RTT*2, 3, 4, 5 (TGG-to-TGC; TGGG-to-TGCG |
| | (SEQ ID NO: 695) | PAM silencing edit) |
| 696 | TATGGGATCAGGCTTCCACTTTGGAAACTCAGCA | RTT*2, 3, 4, 5 (TGG-to-TGA; TGGG-to-TGAG |
| | (SEQ ID NO: 696) | PAM silencing edit) |
| 697 | GTATGGGATCAGGCTTCCACTTTGGAAACACAGC | RTT*2, 3, 4, 5 (TGG-to-TGT; TGGG-to-TGTG |
| | A (SEQ ID NO: 697) | PAM silencing edit) |
| 698 | GTATGGGATCAGGCTTCCACTTTGGAAACCCAGC | RTT*1, 2, 3, 4, 5, 6 |
| | A (SEQ ID NO: 698) | |
| 699 | GTATGGGATCAGGCTTCCACTTTGGAAACGCAGC | RTT*2, 3, 4, 5 (TGG-to-TGC; TGGG-to-TGCG |
| | A (SEQ ID NO: 699) | PAM silencing edit) |
| 700 | GTATGGGATCAGGCTTCCACTTTGGAAACTCAGC | RTT*2, 3, 4, 5 (TGG-to-TGA; TGGG-to-TGAG |
| | A (SEQ ID NO: 700) | PAM silencing edit) |
| 701 | TGTATGGGATCAGGCTTCCACTTTGGAAACACAG | RTT*2, 3, 4, 5 (TGG-to-TGT; TGGG-to-TGTG |
| | CA (SEQ ID NO: 701) | PAM silencing edit) |
| 702 | TGTATGGGATCAGGCTTCCACTTTGGAAACCCAG | RTT*1, 2, 3, 4, 5, 6 |
| | CA (SEQ ID NO: 702) | |
| 703 | TGTATGGGATCAGGCTTCCACTTTGGAAACGCAG | RTT*2, 3, 4, 5 (TGG-to-TGC; TGGG-to-TGCG |
| | CA (SEQ ID NO: 703) | PAM silencing edit) |
| 704 | TGTATGGGATCAGGCTTCCACTTTGGAAACTCAG | RTT*2, 3, 4, 5 (TGG-to-TGA; TGGG-to-TGAG |
| | CA (SEQ ID NO: 704) | PAM silencing edit) |
| 705 | CTGTATGGGATCAGGCTTCCACTTTGGAAACACA | RTT*2, 3, 4, 5 (TGG-to-TGT; TGGG-to-TGTG |
| | GCA (SEQ ID NO: 705) | PAM silencing edit) |
| 706 | CTGTATGGGATCAGGCTTCCACTTTGGAAACCCA | RTT*1, 2, 3, 4, 5, 6 |
| | GCA (SEQ ID NO: 706) | |
| 707 | CTGTATGGGATCAGGCTTCCACTTTGGAAACGCA | RTT*2, 3, 4, 5 (TGG-to-TGC; TGGG-to-TGCG |
| | GCA (SEQ ID NO: 707) | PAM silencing edit) |
| 708 | CTGTATGGGATCAGGCTTCCACTTTGGAAACTCA | RTT*2, 3, 4, 5 (TGG-to-TGA; TGGG-to-TGAG |
| | GCA (SEQ ID NO: 708) | PAM silencing edit) |
| 709 | TCTGTATGGGATCAGGCTTCCACTTTGGAAACAC | RTT*2, 3, 4, 5 (TGG-to-TGT; TGGG-to-TGTG |
| | AGCA (SEQ ID NO: 709) | PAM silencing edit) |
| 710 | TCTGTATGGGATCAGGCTTCCACTTTGGAAACCC | RTT*1, 2, 3, 4, 5, 6 |
| | AGCA (SEQ ID NO: 710) | |
| 711 | TCTGTATGGGATCAGGCTTCCACTTTGGAAACGC | RTT*2, 3, 4, 5 (TGG-to-TGC; TGGG-to-TGCG |
| | AGCA (SEQ ID NO: 711) | PAM silencing edit) |
| 712 | TCTGTATGGGATCAGGCTTCCACTTTGGAAACTC | RTT*2, 3, 4, 5 (TGG-to-TGA; TGGG-to-TGAG |
| | AGCA (SEQ ID NO: 712) | PAM silencing edit) |
| 713 | TTCTGTATGGGATCAGGCTTCCACTTTGGAAACA | RTT*2, 3, 4, 5 (TGG-to-TGT; TGGG-to-TGTG |
| | CAGCA (SEQ ID NO: 713) | PAM silencing edit) |
| 714 | TTCTGTATGGGATCAGGCTTCCACTTTGGAAACC | RTT*1, 2, 3, 4, 5, 6 |
| | CAGCA (SEQ ID NO: 714) | |
| 715 | TTCTGTATGGGATCAGGCTTCCACTTTGGAAACG | RTT*2, 3, 4, 5 (TGG-to-TGC; TGGG-to-TGCG |
| | CAGCA (SEQ ID NO: 715) | PAM silencing edit) |
| 716 | TTCTGTATGGGATCAGGCTTCCACTTTGGAAACT | RTT*2, 3, 4, 5 (TGG-to-TGA; TGGG-to-TGAG |
| | CAGCA (SEQ ID NO: 716) | PAM silencing edit) |
| 717 | TTTCTGTATGGGATCAGGCTTCCACTTTGGAAAC | RTT*2, 3, 4, 5 (TGG-to-TGT; TGGG-to-TGTG |
| | ACAGCA (SEQ ID NO: 717) | PAM silencing edit) |
| 718 | TTTCTGTATGGGATCAGGCTTCCACTTTGGAAAC | RTT*1, 2, 3, 4, 5, 6 |
| | CCAGCA (SEQ ID NO: 718) | |
| 719 | TTTCTGTATGGGATCAGGCTTCCACTTTGGAAAC | RTT*2, 3, 4, 5 (TGG-to-TGC; TGGG-to-TGCG |
| | GCAGCA (SEQ ID NO: 719) | PAM silencing edit) |
| 720 | TTTCTGTATGGGATCAGGCTTCCACTTTGGAAAC | RTT*2, 3, 4, 5 (TGG-to-TGA; TGGG-to-TGAG |
| | TCAGCA (SEQ ID NO: 720) | PAM silencing edit) |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b*1 ngRNA spacer (GG PAM) |
| 126 | ACCTTTTCGCTTTTTCCAAG (SEQ ID NO: 126) | PE3 ngRNA spacer (ATGG PAM) |
| 127 | AGAAGCTTTCTGTATGGGAT (SEQ ID NO: 127) | PE3b*2 ngRNA spacer (CAGG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b*3 ngRNA spacer (TG; TGG PAM) |
| 29 | CCTTTTCGCTTTTTCCAAGA (SEQ ID NO: 29) | PE3 ngRNA spacer (TG; TGG PAM) |
| 131 | CTTTCTTGTGATTATCAGTT (SEQ ID NO: 131) | PE3 ngRNA spacer (TTGG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b*4 ngRNA spacer (AG; AGG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG; TGG PAM) |
| 36 | GGCTTCCACTTTGGAAACCC (SEQ ID NO: 36) | PE3 ngRNA spacer (AG PAM) |
| 37 | GTGAAGGGACATCACCTTTT (SEQ ID NO: 37) | PE3 ngRNA spacer (CG PAM) |
| 132 | TATGGGATCAGGCTTCCACT (SEQ ID NO: 132) | PE3b*5 ngRNA spacer (TTGG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 40 | TCCACTTTGGAAACCCAGCA (SEQ ID NO: 40) | PE3 ngRNA spacer (AG PAM) |
| 41 | TGATTATCAGTTTTGGATGC (SEQ ID NO: 41) | PE3 ngRNA spacer (AG PAM) |
| 134 | TGGATGCAGAAGCTTTCTGT (SEQ ID NO: 134) | PE3 ngRNA spacer (ATGG PAM) |
| 43 | TGGGATCAGGCTTCCACTTT (SEQ ID NO: 43) | PE3b*6 ngRNA spacer (GG PAM) |
| 44 | TTATCAGTTTTGGATGCAGA (SEQ ID NO: 44) | PE3 ngRNA spacer (AG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG; TGG PAM) |
| 48 | TTTTGGATGCAGAAGCTTTC (SEQ ID NO: 48) | PE3 ngRNA spacer (TG PAM) |
| 721 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCT (SEQ ID NO: 721) | |
| 722 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGT (SEQ ID NO: 722) | |
| 723 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCT (SEQ ID NO: 723) | |
| 724 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTCT (SEQ ID NO: 724) | |
| 725 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGT (SEQ ID NO: 725) | |
| 726 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCT (SEQ ID NO: 726) | |
| 727 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCATGG | |
| | GATCAGGCTTCCACTTTGGAAACCCAGCAAGAC | |
| | ACCTTTTT (SEQ ID NO: 727) | |
| 728 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTCTTC (SEQ ID NO: 728) | |
| 729 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTCT (SEQ ID NO: 729) | |
| 730 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGT (SEQ ID NO: 730) | |
| 731 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCT (SEQ ID NO: 731) | |
| 732 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCATGG | |
| | GATCAGGCTTCCACTTTGGAAACCCAGCAAGAC | |
| | ACCTGTTTTT (SEQ ID NO: 732) | |
| 733 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCGTAT | |
| | GGGATCAGGCTTCCACTTTGGAAACCCAGCAAG | |
| | ACACCTTTTT (SEQ ID NO: 733) | |
| 734 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTCTTC (SEQ ID NO: 734) | |
| 735 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTCT (SEQ ID NO: 735) | |
| 736 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGT (SEQ ID NO: 736) | |
| 737 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCATGG | |
| | GATCAGGCTTCCACTTTGGAAACCCAGCAAGAC | |
| | ACCTGTCTTTTT (SEQ ID NO: 737) | |
| 738 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCGTAT | |
| | GGGATCAGGCTTCCACTTTGGAAACCCAGCAAG | |
| | ACACCTGTTTTT (SEQ ID NO: 738) | |
| 739 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTCTGT | |
| | ATGGGATCAGGCTTCCACTTTGGAAACCCAGCA | |
| | AGACACCTTTTT (SEQ ID NO: 739) | |
| 740 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTCTTC (SEQ ID NO: 740) | |
| 741 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTCT (SEQ ID NO: 741) | |
| 742 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCATGG | |
| | GATCAGGCTTCCACTTTGGAAACCCAGCAAGAC | |
| | ACCTGTCTTCTTTT (SEQ ID NO: 742) | |
| 743 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCGTAT | |
| | GGGATCAGGCTTCCACTTTGGAAACCCAGCAAG | |
| | ACACCTGTCTTTTT (SEQ ID NO: 743) | |
| 744 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTCTGT | |
| | ATGGGATCAGGCTTCCACTTTGGAAACCCAGCA | |
| | AGACACCTGTTTTT (SEQ ID NO: 744) | |
| 745 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTTTCT | |
| | GTATGGGATCAGGCTTCCACTTTGGAAACCCAGC | |
| | AAGACACCTTTTT (SEQ ID NO: 745) | |
| 746 | CGTGAAGACAGGTGTCTTGCGTTTTAGAGCTAGA | pegRNA*1, 2, 3, 4, 5, 6 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTCTTC (SEQ ID NO: 746) | |
| 747 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCGTAT | |
| | GGGATCAGGCTTCCACTTTGGAAACCCAGCAAG | |
| | ACACCTGTCTTCTTTT (SEQ ID NO: 747) | |
| 748 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTCTGT | |
| | ATGGGATCAGGCTTCCACTTTGGAAACCCAGCA | |
| | AGACACCTGTCTTTTT (SEQ ID NO: 748) | |
| 749 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTTTCT | |
| | GTATGGGATCAGGCTTCCACTTTGGAAACCCAGC | |
| | AAGACACCTGTTTTT (SEQ ID NO: 749) | |
| 750 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTCTGT | |
| | ATGGGATCAGGCTTCCACTTTGGAAACCCAGCA | |
| | AGACACCTGTCTTCTTTT (SEQ ID NO: 750) | |
| 751 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTTTCT | |
| | GTATGGGATCAGGCTTCCACTTTGGAAACCCAGC | |
| | AAGACACCTGTCTTTTT (SEQ ID NO: 751) | |
| 752 | GCGTGAAGACAGGTGTCTTGCGTTTTAGAGCTAG | pegRNA*1, 2, 3, 4, 5, 6, contains end |
| | AAATAGCAAGTTAAAATAAGGCTAGTCCGTTATC | adaptations |
| | AACTTGAAAAAGTGGCACCGAGTCGGTGCTTTCT | |
| | GTATGGGATCAGGCTTCCACTTTGGAAACCCAGC | |
| | AAGACACCTGTCTTCTTTT (SEQ ID NO: 752) | |
| 49 | TTTCTTGTGATTATCAGTTTGTTTTAGAGCTAGAA | PE3 ngRNA (TG; TGG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b*4 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b*3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG; TGG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b*4 ngRNA (AG; AGG PAM), contains |
| | AATAGCAAGTTAAAATAAGGCTAGTCCGTTATCA | end adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG; TGG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b*3 ngRNA (TG; TGG PAM), contains |
| | AAATAGCAAGTTAAAATAAGGCTAGTCCGTTATC | end adaptations |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b*4 ngRNA (AG; AGG PAM), contains |
| | AATAGCAAGTTAAAATAAGGCTAGTCCGTTATCA | linker sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b*3 ngRNA (TG; TGG PAM), contains |
| | AAATAGCAAGTTAAAATAAGGCTAGTCCGTTATC | linker sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 60) | |

TABLE 11

| Sequence | | |
|---|---|---|
| Number | Sequence | Description |
| 753 | AGCCACGTGAAGACAGG (SEQ ID NO: 753) | PEgRNA spacer (TG PAM) |
| 754 | GAGCCACGTGAAGACAGG (SEQ ID NO: 754) | PEgRNA spacer (TG PAM) |
| 755 | TGAGCCACGTGAAGACAGG (SEQ ID NO: 755) | PEgRNA spacer (TG PAM) |
| 205 | CTGAGCCACGTGAAGACAGG (SEQ ID NO: 205) | PEgRNA spacer (TG PAM) |
| 756 | ACTGAGCCACGTGAAGACAGG (SEQ ID NO: 756) | PEgRNA spacer (TG PAM) |
| 757 | AACTGAGCCACGTGAAGACAGG (SEQ ID NO: 757) | PEgRNA spacer (TG PAM) |
| 758 | GTCTT | PBS |
| 759 | GTCTTC | PBS |
| 760 | GTCTTCA | PBS |
| 761 | GTCTTCAC | PBS |
| 762 | GTCTTCACG | PBS |
| 763 | GTCTTCACGT (SEQ ID NO: 763) | PBS |
| 764 | GTCTTCACGTG (SEQ ID NO: 764) | PBS |
| 765 | GTCTTCACGTGG (SEQ ID NO: 765) | PBS |
| 766 | GTCTTCACGTGGC (SEQ ID NO: 766) | PBS |
| 767 | GTCTTCACGTGGCT (SEQ ID NO: 767) | PBS |
| 768 | GTCTTCACGTGGCTC (SEQ ID NO: 768) | PBS |
| 769 | GTCTTCACGTGGCTCA (SEQ ID NO: 769) | PBS |
| 770 | GTCTTCACGTGGCTCAG (SEQ ID NO: 770) | PBS |
| 771 | GTCTTCACGTGGCTCAGT (SEQ ID NO: 771) | PBS |
| 772 | GTCTTCACGTGGCTCAGTT (SEQ ID NO: 772) | PBS |
| 773 | GATCAGGCTTCCACTTTGGAAACCCAGCAAGAC | RTT*1, 2, 3, 5 |
| | ACCT (SEQ ID NO: 773) | |
| 774 | GATCAGGCTTCCACTTTGGAAACCCAGCAAGAT | RTT*1, 2, 3, 4, 5 (TG-to-TA PAM silencing |
| | ACCT (SEQ ID NO: 774) | edit) |
| 775 | GGATCAGGCTTCCACTTTGGAAACCCAGCAAGA | RTT*1, 2, 3, 5 |
| | CACCT (SEQ ID NO: 775) | |
| 776 | GGATCAGGCTTCCACTTTGGAAACCCAGCAAGA | RTT*1, 2, 3, 4, 5 (TG-to-TA PAM silencing |
| | TACCT (SEQ ID NO: 776) | edit) |
| 777 | GGGATCAGGCTTCCACTTTGGAAACCCAGCAAG | RTT*1, 2, 3, 5 |
| | ACACCT (SEQ ID NO: 777) | |
| 778 | GGGATCAGGCTTCCACTTTGGAAACCCAGCAAG | RTT*1, 2, 3, 4, 5 (TG-to-TA PAM silencing |
| | ATACCT (SEQ ID NO: 778) | edit) |
| 779 | TGGGATCAGGCTTCCACTTTGGAAACCCAGCAA | RTT*1, 2, 3, 5 |
| | GACACCT (SEQ ID NO: 779) | |
| 780 | TGGGATCAGGCTTCCACTTTGGAAACCCAGCAA | RTT*1, 2, 3, 4, 5 (TG-to-TA PAM silencing |
| | GATACCT (SEQ ID NO: 780) | edit) |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b*1 ngRNA spacer (GG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b*2 ngRNA spacer (TG PAM) |
| 29 | CCTTTTCGCTTTTTCCAAGA (SEQ ID NO: 29) | PE3 ngRNA spacer (TG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b*3 ngRNA spacer (AG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG PAM) |
| 36 | GGCTTCCACTTTGGAAACCC (SEQ ID NO: 36) | PE3 ngRNA spacer (AG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 40 | TCCACTTTGGAAACCCAGCA (SEQ ID NO: 40) | PE3 ngRNA spacer (AG PAM) |
| 41 | TGATTATCAGTTTTGGATGC (SEQ ID NO: 41) | PE3 ngRNA spacer (AG PAM) |
| 42 | TGGAAACCCAGCAAGACACC (SEQ ID NO: 42) | PE3 ngRNA spacer (TG PAM) |
| 781 | TGGAAACCCAGCAAGATACC (SEQ ID NO: 781) | PE3b*4 ngRNA spacer (TG PAM) |
| 43 | TGGGATCAGGCTTCCACTTT (SEQ ID NO: 43) | PE3b*5 ngRNA spacer (GG PAM) |
| 44 | TTATCAGTTTTGGATGCAGA (SEQ ID NO: 44) | PE3 ngRNA spacer (AG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b*3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b*2 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b*3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b*2 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b*3 ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b*2 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 60) | |

TABLE 12

| Sequence | | |
|---|---|---|
| Number | Sequence | Description |
| 782 | GGTTTCCAAAGTGGAAG (SEQ ID NO: 782) | PEgRNA spacer (CCTGAT PAM) |
| 783 | GGGTTTCCAAAGTGGAAG (SEQ ID NO: 783) | PEgRNA spacer (CCTGAT PAM) |
| 784 | TGGGTTTCCAAAGTGGAAG (SEQ ID NO: 784) | PEgRNA spacer (CCTGAT PAM) |
| 413 | CTGGGTTTCCAAAGTGGAAG (SEQ ID NO: 413) | PEgRNA spacer (CCTGAT PAM) |
| 785 | GCTGGGTTTCCAAAGTGGAAG (SEQ ID NO: 785) | PEgRNA spacer (CCTGAT PAM) |
| 786 | TGCTGGGTTTCCAAAGTGGAAG (SEQ ID NO: 786) | PEgRNA spacer (CCTGAT PAM) |
| 787 | CCACT | PBS |
| 788 | CCACTT | PBS |
| 789 | CCACTTT | PBS |
| 790 | CCACTTTG | PBS |
| 791 | CCACTTTGG | PBS |
| 792 | CCACTTTGGA (SEQ ID NO: 792) | PBS |
| 793 | CCACTTTGGAA (SEQ ID NO: 793) | PBS |
| 794 | CCACTTTGGAAA (SEQ ID NO: 794) | PBS |
| 795 | CCACTTTGGAAAC (SEQ ID NO: 795) | PBS |
| 796 | CCACTTTGGAAACC (SEQ ID NO: 796) | PBS |
| 797 | CCACTTTGGAAACCC (SEQ ID NO: 797) | PBS |
| 798 | CCACTTTGGAAACCCA (SEQ ID NO: 798) | PBS |
| 799 | CCACTTTGGAAACCCAG (SEQ ID NO: 799) | PBS |
| 800 | CCACTTTGGAAACCCAGC (SEQ ID NO: 800) | PBS |
| 801 | CCACTTTGGAAACCCAGCA (SEQ ID NO: 801) | PBS |
| 802 | GATCAGGCTT (SEQ ID NO: 802) | RTT |
| 803 | GGATCAGGCTT (SEQ ID NO: 803) | RTT |
| 804 | GGGATCAGGCTT (SEQ ID NO: 804) | RTT |
| 805 | TGGGATCAGGCTT (SEQ ID NO: 805) | RTT |
| 806 | ATGGGATCAGGCTT (SEQ ID NO: 806) | RTT |
| 807 | TATGGGATCAGGCTT (SEQ ID NO: 807) | RTT |
| 808 | GTATGGGATCAGGCTT (SEQ ID NO: 808) | RTT |
| 809 | TGTATGGGATCAGGCTT (SEQ ID NO: 809) | RTT |
| 810 | CTGTATGGGATCAGGCTT (SEQ ID NO: 810) | RTT |
| 811 | TCTGTATGGGATCAGGCTT (SEQ ID NO: 811) | RTT |
| 812 | TTCTGTATGGGATCAGGCTT (SEQ ID NO: 812) | RTT |
| 813 | TTTCTGTATGGGATCAGGCTT (SEQ ID NO: 813) | RTT |
| 814 | CTTTCTGTATGGGATCAGGCTT (SEQ ID NO: 814) | RTT |
| 815 | GCTTTCTGTATGGGATCAGGCTT (SEQ ID NO: 815) | RTT |
| 816 | AGCTTTCTGTATGGGATCAGGCTT (SEQ ID NO: | RTT |
| | 816) | |
| 817 | AAGCTTTCTGTATGGGATCAGGCTT (SEQ ID NO: | RTT |
| | 817) | |
| 818 | GAAGCTTTCTGTATGGGATCAGGCTT (SEQ ID | RTT |
| | NO: 818) | |
| 819 | AGAAGCTTTCTGTATGGGATCAGGCTT (SEQ ID | RTT |
| | NO: 819) | |
| 820 | CAGAAGCTTTCTGTATGGGATCAGGCTT (SEQ ID | RTT |
| | NO: 820) | |
| 821 | GCAGAAGCTTTCTGTATGGGATCAGGCTT (SEQ | RTT |
| | ID NO: 821) | |
| 822 | TGCAGAAGCTTTCTGTATGGGATCAGGCTT (SEQ | RTT |
| | ID NO: 822) | |
| 823 | ATGCAGAAGCTTTCTGTATGGGATCAGGCTT | RTT |
| | (SEQ ID NO: 823) | |
| 824 | GATGCAGAAGCTTTCTGTATGGGATCAGGCTT | RTT |
| | (SEQ ID NO: 824) | |
| 825 | GGATGCAGAAGCTTTCTGTATGGGATCAGGCTT | RTT |
| | (SEQ ID NO: 825) | |
| 826 | TGGATGCAGAAGCTTTCTGTATGGGATCAGGCTT | RTT |
| | (SEQ ID NO: 826) | |
| 827 | TTGGATGCAGAAGCTTTCTGTATGGGATCAGGCT | RTT |
| | T (SEQ ID NO: 827) | |
| 828 | TTTGGATGCAGAAGCTTTCTGTATGGGATCAGGC | RTT |
| | TT (SEQ ID NO: 828) | |
| 829 | TTTTGGATGCAGAAGCTTTCTGTATGGGATCAGG | RTT |
| | CTT (SEQ ID NO: 829) | |
| 830 | GTTTTGGATGCAGAAGCTTTCTGTATGGGATCAG | RTT |
| | GCTT (SEQ ID NO: 830) | |
| 831 | AGTTTTGGATGCAGAAGCTTTCTGTATGGGATCA | RTT |
| | GGCTT (SEQ ID NO: 831) | |
| 832 | CAGTTTTGGATGCAGAAGCTTTCTGTATGGGATC | RTT |
| | AGGCTT (SEQ ID NO: 832) | |
| 25 | AAGATTCAGTAGATCTTTCT (SEQ ID NO: 25) | PE3 ngRNA spacer (TGTGAT PAM) |
| 128 | AGTAGATCTTTCTTGTGATT (SEQ ID NO: 128) | PE3 ngRNA spacer (ATCAGT PAM) |
| 129 | CATCACCTTTTCGCTTTTTC (SEQ ID NO: 129) | PE3 ngRNA spacer (CAAGAT PAM) |
| 130 | CCAAGATGGCTCAAGATTCA (SEQ ID NO: 130) | PE3 ngRNA spacer (GTAGAT PAM) |
| 131 | CTTTCTTGTGATTATCAGTT (SEQ ID NO: 131) | PE3 ngRNA spacer (TTGGAT PAM) |
| 133 | TCGCTTTTTCCAAGATGGCT (SEQ ID NO: 133) | PE3 ngRNA spacer (CAAGAT PAM) |
| 134 | TGGATGCAGAAGCTTTCTGT (SEQ ID NO: 134) | PE3 ngRNA spacer (ATGGAT PAM) |
| 135 | TTTTCCAAGATGGCTCAAGA (SEQ ID NO: 135) | PE3 ngRNA spacer (TTCAGT PAM) |

TABLE 13

| Sequence | | |
|---|---|---|
| Number | Sequence | Description |
| 833 | TCTTGTGATTATCAGTT (SEQ ID NO: 833) | PEgRNA spacer (TTGG; TTGGAT PAM) |
| 834 | TTCTTGTGATTATCAGTT (SEQ ID NO: 834) | PEgRNA spacer (TTGG; TTGGAT PAM) |
| 835 | TTTCTTGTGATTATCAGTT (SEQ ID NO: 835) | PEgRNA spacer (TTGG; TTGGAT PAM) |
| 131 | CTTTCTTGTGATTATCAGTT (SEQ ID NO: 131) | PEgRNA spacer (TTGG; TTGGAT PAM) |
| 836 | TCTTTCTTGTGATTATCAGTT (SEQ ID NO: 836) | PEgRNA spacer (TTGG; TTGGAT PAM) |
| 837 | ATCTTTCTTGTGATTATCAGTT (SEQ ID NO: 837) | PEgRNA spacer (TTGG; TTGGAT PAM) |
| 838 | TGATA | PBS |
| 839 | TGATAA | PBS |
| 840 | TGATAAT | PBS |
| 841 | TGATAATC | PBS |
| 842 | TGATAATCA | PBS |
| 843 | TGATAATCAC (SEQ ID NO: 843) | PBS |
| 844 | TGATAATCACA (SEQ ID NO: 844) | PBS |
| 845 | TGATAATCACAA (SEQ ID NO: 845) | PBS |
| 846 | TGATAATCACAAG (SEQ ID NO: 846) | PBS |
| 847 | TGATAATCACAAGA (SEQ ID NO: 847) | PBS |
| 848 | TGATAATCACAAGAA (SEQ ID NO: 848) | PBS |
| 849 | TGATAATCACAAGAAA (SEQ ID NO: 849) | PBS |
| 850 | TGATAATCACAAGAAAG (SEQ ID NO: 850) | PBS |
| 851 | TGATAATCACAAGAAAGA (SEQ ID NO: 851) | PBS |
| 852 | TGATAATCACAAGAAAGAT (SEQ ID NO: 852) | PBS |
| 853 | CCCATACAGAAAGCTTCTGCATCCAAAAC (SEQ | RTT |
| | ID NO: 853) | |
| 854 | TCCCATACAGAAAGCTTCTGCATCCAAAAC (SEQ | RTT |
| | ID NO: 854) | |
| 855 | ATCCCATACAGAAAGCTTCTGCATCCAAAAC | RTT |
| | (SEQ ID NO: 855) | |
| 856 | GATCCCATACAGAAAGCTTCTGCATCCAAAAC | RTT |
| | (SEQ ID NO: 856) | |
| 857 | TGATCCCATACAGAAAGCTTCTGCATCCAAAAC | RTT |
| | (SEQ ID NO: 857) | |
| 858 | CTGATCCCATACAGAAAGCTTCTGCATCCAAAAC | RTT |
| | (SEQ ID NO: 858) | |
| 859 | CCTGATCCCATACAGAAAGCTTCTGCATCCAAAA | RTT |
| | C (SEQ ID NO: 859) | |
| 860 | GCCTGATCCCATACAGAAAGCTTCTGCATCCAAA | RTT |
| | AC (SEQ ID NO: 860) | |
| 861 | AGCCTGATCCCATACAGAAAGCTTCTGCATCCAA | RTT |
| | AAC (SEQ ID NO: 861) | |
| 862 | AAGCCTGATCCCATACAGAAAGCTTCTGCATCCA | RTT |
| | AAAC (SEQ ID NO: 862) | |
| 863 | GAAGCCTGATCCCATACAGAAAGCTTCTGCATCC | RTT |
| | AAAAC (SEQ ID NO: 863) | |
| 864 | GGAAGCCTGATCCCATACAGAAAGCTTCTGCATC | RTT |
| | CAAAAC (SEQ ID NO: 864) | |
| 410 | AAGCTTCTGCATCCAAAACT (SEQ ID NO: 410) | PE3 ngRNA spacer (GATAAT PAM) |
| 480 | AAGGCTTCATACATCTTCCT (SEQ ID NO: 480) | PE3 ngRNA spacer (TAGG PAM) |
| 64 | ACGTGAAGACAGGTGTCTTG (SEQ ID NO: 64) | PE3 ngRNA spacer (CTGG; CTGGGT PAM) |
| 411 | AGAAAGCTTCTGCATCCAAA (SEQ ID NO: 411) | PE3 ngRNA spacer (ACTGAT PAM) |
| 482 | CACTACTTACCATCTCTTTC (SEQ ID NO: 482) | PE3 ngRNA spacer (AAGG PAM) |
| 412 | CAGGTGTCTTGCTGGGTTTC (SEQ ID NO: 412) | PE3 ngRNA spacer (CAAAGT PAM) |
| 203 | CGTGAAGACAGGTGTCTTGC (SEQ ID NO: 203) | PE3 ngRNA spacer (TGGG PAM) |
| 413 | CTGGGTTTCCAAAGTGGAAG (SEQ ID NO: 413) | PE3 ngRNA spacer (CCTGAT PAM) |
| 485 | GGAACTGAGCCACGTGAAGA (SEQ ID NO: 485) | PE3 ngRNA spacer (CAGG PAM) |
| 542 | TACATCTTCCTTAGGAACTC (SEQ ID NO: 542) | PE3 ngRNA spacer (CTGG PAM) |
| 414 | TGGAACTGAGCCACGTGAAG (SEQ ID NO: 414) | PE3 ngRNA spacer (ACAGGT PAM) |
| 543 | TGTCTTGCTGGGTTTCCAAA (SEQ ID NO: 543) | PE3 ngRNA spacer (GTGG PAM) |
| 325 | CGTGAAGACAGGTGTCTTGCGTTTTAGAGCTAGA | PE3 ngRNA (TGGG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 333 | GCGTGAAGACAGGTGTCTTGCGTTTTAGAGCTAG | PE3 ngRNA (TGGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 340 | GCGTGAAGACAGGTGTCTTGCGTTTTAGAGCTAG | PE3 ngRNA (TGGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |

TABLE 14

| Sequence | | |
|---|---|---|
| Number | Sequence | Description |
| 865 | GCAGAAGCTTTCTGTAT (SEQ ID NO: 865) | PEgRNA spacer (GG; GGA PAM) |
| 866 | TGCAGAAGCTTTCTGTAT (SEQ ID NO: 866) | PEgRNA spacer (GG; GGA PAM) |
| 867 | ATGCAGAAGCTTTCTGTAT (SEQ ID NO: 867) | PEgRNA spacer (GG; GGA PAM) |
| 32 | GATGCAGAAGCTTTCTGTAT (SEQ ID NO: 32) | PEgRNA spacer (GG; GGA PAM) |
| 868 | GGATGCAGAAGCTTTCTGTAT (SEQ ID NO: 868) | PEgRNA spacer (GG; GGA PAM) |
| 869 | TGGATGCAGAAGCTTTCTGTAT (SEQ ID NO: 869) | PEgRNA spacer (GG; GGA PAM) |
| 870 | CAGAA | PBS |
| 871 | CAGAAA | PBS |
| 872 | CAGAAAG | PBS |
| 873 | CAGAAAGC | PBS |
| 874 | CAGAAAGCT | PBS |
| 875 | CAGAAAGCTT (SEQ ID NO: 875) | PBS |
| 876 | CAGAAAGCTTC (SEQ ID NO: 876) | PBS |
| 877 | CAGAAAGCTTCT (SEQ ID NO: 877) | PBS |
| 878 | CAGAAAGCTTCTG (SEQ ID NO: 878) | PBS |
| 879 | CAGAAAGCTTCTGC (SEQ ID NO: 879) | PBS |
| 880 | CAGAAAGCTTCTGCA (SEQ ID NO: 880) | PBS |
| 881 | CAGAAAGCTTCTGCAT (SEQ ID NO: 881) | PBS |
| 882 | CAGAAAGCTTCTGCATC (SEQ ID NO: 882) | PBS |
| 883 | CAGAAAGCTTCTGCATCC (SEQ ID NO: 883) | PBS |
| 884 | CAGAAAGCTTCTGCATCCA (SEQ ID NO: 884) | PBS |
| 885 | TGATCCCATA (SEQ ID NO: 885) | RTT |
| 886 | CTGATCCCATA (SEQ ID NO: 886) | RTT |
| 887 | CCTGATCCCATA (SEQ ID NO: 887) | RTT |
| 888 | GCCTGATCCCATA (SEQ ID NO: 888) | RTT |
| 889 | AGCCTGATCCCATA (SEQ ID NO: 889) | RTT |
| 890 | AAGCCTGATCCCATA (SEQ ID NO: 890) | RTT |
| 891 | GAAGCCTGATCCCATA (SEQ ID NO: 891) | RTT |
| 892 | GGAAGCCTGATCCCATA (SEQ ID NO: 892) | RTT |
| 893 | TGGAAGCCTGATCCCATA (SEQ ID NO: 893) | RTT |
| 894 | GTGGAAGCCTGATCCCATA (SEQ ID NO: 894) | RTT |
| 481 | AGTGGAAGCCTGATCCCATA (SEQ ID NO: 481) | RTT |
| 895 | AAGTGGAAGCCTGATCCCATA (SEQ ID NO: 895) | RTT |
| 896 | AAAGTGGAAGCCTGATCCCATA (SEQ ID NO: 896) | RTT |
| 897 | CAAAGTGGAAGCCTGATCCCATA (SEQ ID NO: | RTT |
| | 897) | |
| 898 | CCAAAGTGGAAGCCTGATCCCATA (SEQ ID NO: | RTT |
| | 898) | |
| 899 | TCCAAAGTGGAAGCCTGATCCCATA (SEQ ID NO: | RTT |
| | 899) | |
| 900 | TTCCAAAGTGGAAGCCTGATCCCATA (SEQ ID | RTT |
| | NO: 900) | |
| 901 | TTTCCAAAGTGGAAGCCTGATCCCATA (SEQ ID | RTT |
| | NO: 901) | |
| 902 | GTTTCCAAAGTGGAAGCCTGATCCCATA (SEQ ID | RTT |
| | NO: 902) | |
| 903 | GGTTTCCAAAGTGGAAGCCTGATCCCATA (SEQ | RTT |
| | ID NO: 903) | |
| 904 | GGGTTTCCAAAGTGGAAGCCTGATCCCATA (SEQ | RTT |
| | ID NO: 904) | |
| 905 | TGGGTTTCCAAAGTGGAAGCCTGATCCCATA | RTT |
| | (SEQ ID NO: 905) | |
| 906 | CTGGGTTTCCAAAGTGGAAGCCTGATCCCATA | RTT |
| | (SEQ ID NO: 906) | |
| 907 | GCTGGGTTTCCAAAGTGGAAGCCTGATCCCATA | RTT |
| | (SEQ ID NO: 907) | |
| 908 | TGCTGGGTTTCCAAAGTGGAAGCCTGATCCCATA | RTT |
| | (SEQ ID NO: 908) | |
| 909 | TTGCTGGGTTTCCAAAGTGGAAGCCTGATCCCAT | RTT |
| | A (SEQ ID NO: 909) | |
| 910 | CTTGCTGGGTTTCCAAAGTGGAAGCCTGATCCCA | RTT |
| | TA (SEQ ID NO: 910) | |
| 911 | TCTTGCTGGGTTTCCAAAGTGGAAGCCTGATCCC | RTT |
| | ATA (SEQ ID NO: 911) | |
| 912 | GTCTTGCTGGGTTTCCAAAGTGGAAGCCTGATCC | RTT |
| | CATA (SEQ ID NO: 912) | |
| 913 | TGTCTTGCTGGGTTTCCAAAGTGGAAGCCTGATC | RTT |
| | CCATA (SEQ ID NO: 913) | |
| 914 | GTGTCTTGCTGGGTTTCCAAAGTGGAAGCCTGAT | RTT |
| | CCCATA (SEQ ID NO: 914) | |
| 4 | AACTGAGCCACGTGAAGACA (SEQ ID NO: 4) | PE3 ngRNA spacer (GG PAM) |
| 195 | ACATCTTCCTTAGGAACTCC (SEQ ID NO: 195) | PE3 ngRNA spacer (TG PAM) |
| 196 | ACTACTTACCATCTCTTTCA (SEQ ID NO: 196) | PE3 ngRNA spacer (AG PAM) |
| 197 | ACTCCTGGAACTGAGCCACG (SEQ ID NO: 197) | PE3 ngRNA spacer (TG; TGA PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG; GGA PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG; AGA PAM) |
| 203 | CGTGAAGACAGGTGTCTTGC (SEQ ID NO: 203) | PE3 ngRNA spacer (TG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 207 | GAAAATAATTTCATTATTCT (SEQ ID NO: 207) | PE3 ngRNA spacer (GG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 210 | GGCTTCATACATCTTCCTTA (SEQ ID NO: 210) | PE3 ngRNA spacer (GG; GGA PAM) |
| 211 | GGGTTTCCAAAGTGGAAGCC (SEQ ID NO: 211) | PE3 ngRNA spacer (TG; TGA PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b ngRNA spacer (AG; AGA PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG; TGA PAM) |
| 216 | TCTTGCTGGGTTTCCAAAGT (SEQ ID NO: 216) | PE3 ngRNA spacer (GG; GGA PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 15

| Sequence | | |
|---|---|---|
| Number | Sequence | Description |
| 915 | TGCAGAAGCTTTCTGTA (SEQ ID NO: 915) | PEgRNA spacer (TG; TGG PAM) |
| 916 | ATGCAGAAGCTTTCTGTA (SEQ ID NO: 916) | PEgRNA spacer (TG; TGG PAM) |
| 917 | GATGCAGAAGCTTTCTGTA (SEQ ID NO: 917) | PEgRNA spacer (TG; TGG PAM) |
| 35 | GGATGCAGAAGCTTTCTGTA (SEQ ID NO: 35) | PEgRNA spacer (TG; TGG PAM) |
| 918 | TGGATGCAGAAGCTTTCTGTA (SEQ ID NO: 918) | PEgRNA spacer (TG; TGG PAM) |
| 919 | TTGGATGCAGAAGCTTTCTGTA (SEQ ID NO: 919) | PEgRNA spacer (TG; TGG PAM) |
| 920 | AGAAA | PBS |
| 921 | AGAAAG | PBS |
| 922 | AGAAAGC | PBS |
| 923 | AGAAAGCT | PBS |
| 924 | AGAAAGCTT | PBS |
| 925 | AGAAAGCTTC (SEQ ID NO: 925) | PBS |
| 926 | AGAAAGCTTCT (SEQ ID NO: 926) | PBS |
| 927 | AGAAAGCTTCTG (SEQ ID NO: 927) | PBS |
| 928 | AGAAAGCTTCTGC (SEQ ID NO: 928) | PBS |
| 929 | AGAAAGCTTCTGCA (SEQ ID NO: 929) | PBS |
| 930 | AGAAAGCTTCTGCAT (SEQ ID NO: 930) | PBS |
| 931 | AGAAAGCTTCTGCATC (SEQ ID NO: 931) | PBS |
| 932 | AGAAAGCTTCTGCATCC (SEQ ID NO: 932) | PBS |
| 933 | AGAAAGCTTCTGCATCCA (SEQ ID NO: 933) | PBS |
| 934 | AGAAAGCTTCTGCATCCAA (SEQ ID NO: 934) | PBS |
| 935 | GATCCCATAC (SEQ ID NO: 935) | RTT |
| 936 | TGATCCCATAC (SEQ ID NO: 936) | RTT |
| 937 | CTGATCCCATAC (SEQ ID NO: 937) | RTT |
| 938 | CCTGATCCCATAC (SEQ ID NO: 938) | RTT |
| 939 | GCCTGATCCCATAC (SEQ ID NO: 939) | RTT |
| 940 | AGCCTGATCCCATAC (SEQ ID NO: 940) | RTT |
| 941 | AAGCCTGATCCCATAC (SEQ ID NO: 941) | RTT |
| 942 | GAAGCCTGATCCCATAC (SEQ ID NO: 942) | RTT |
| 943 | GGAAGCCTGATCCCATAC (SEQ ID NO: 943) | RTT |
| 944 | TGGAAGCCTGATCCCATAC (SEQ ID NO: 944) | RTT |
| 384 | GTGGAAGCCTGATCCCATAC (SEQ ID NO: 384) | RTT |
| 945 | AGTGGAAGCCTGATCCCATAC (SEQ ID NO: 945) | RTT |
| 946 | AAGTGGAAGCCTGATCCCATAC (SEQ ID NO: 946) | RTT |
| 947 | AAAGTGGAAGCCTGATCCCATAC (SEQ ID NO: | RTT |
| | 947) | |
| 948 | CAAAGTGGAAGCCTGATCCCATAC (SEQ ID NO: | RTT |
| | 948) | |
| 949 | CCAAAGTGGAAGCCTGATCCCATAC (SEQ ID NO: | RTT |
| | 949) | |
| 950 | TCCAAAGTGGAAGCCTGATCCCATAC (SEQ ID | RTT |
| | NO: 950) | |
| 951 | TTCCAAAGTGGAAGCCTGATCCCATAC (SEQ ID | RTT |
| | NO: 951) | |
| 952 | TTTCCAAAGTGGAAGCCTGATCCCATAC (SEQ ID | RTT |
| | NO: 952) | |
| 953 | GTTTCCAAAGTGGAAGCCTGATCCCATAC (SEQ | RTT |
| | ID NO: 953) | |
| 954 | GGTTTCCAAAGTGGAAGCCTGATCCCATAC (SEQ | RTT |
| | ID NO: 954) | |
| 955 | GGGTTTCCAAAGTGGAAGCCTGATCCCATAC | RTT |
| | (SEQ ID NO: 955) | |
| 956 | TGGGTTTCCAAAGTGGAAGCCTGATCCCATAC | RTT |
| | (SEQ ID NO: 956) | |
| 957 | CTGGGTTTCCAAAGTGGAAGCCTGATCCCATAC | RTT |
| | (SEQ ID NO: 957) | |
| 958 | GCTGGGTTTCCAAAGTGGAAGCCTGATCCCATAC | RTT |
| | (SEQ ID NO: 958) | |
| 959 | TGCTGGGTTTCCAAAGTGGAAGCCTGATCCCATA | RTT |
| | C (SEQ ID NO: 959) | |
| 960 | TTGCTGGGTTTCCAAAGTGGAAGCCTGATCCCAT | RTT |
| | AC (SEQ ID NO: 960) | |
| 961 | CTTGCTGGGTTTCCAAAGTGGAAGCCTGATCCCA | RTT |
| | TAC (SEQ ID NO: 961) | |
| 962 | TCTTGCTGGGTTTCCAAAGTGGAAGCCTGATCCC | RTT |
| | ATAC (SEQ ID NO: 962) | |
| 963 | GTCTTGCTGGGTTTCCAAAGTGGAAGCCTGATCC | RTT |
| | CATAC (SEQ ID NO: 963) | |
| 964 | TGTCTTGCTGGGTTTCCAAAGTGGAAGCCTGATC | RTT |
| | CCATAC (SEQ ID NO: 964) | |
| 4 | AACTGAGCCACGTGAAGACA (SEQ ID NO: 4) | PE3 ngRNA spacer (GG PAM) |
| 195 | ACATCTTCCTTAGGAACTCC (SEQ ID NO: 195) | PE3 ngRNA spacer (TG; TGG PAM) |
| 196 | ACTACTTACCATCTCTTTCA (SEQ ID NO: 196) | PE3 ngRNA spacer (AG; AGG PAM) |
| 197 | ACTCCTGGAACTGAGCCACG (SEQ ID NO: 197) | PE3 ngRNA spacer (TG PAM) |
| 199 | AGGCTTCATACATCTTCCTT (SEQ ID NO: 199) | PE3 ngRNA spacer (AG; AGG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG; TGG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG; AGG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG; TGG PAM) |
| 213 | GTGAAGACAGGTGTCTTGCT (SEQ ID NO: 213) | PE3 ngRNA spacer (GG; GGG PAM) |
| 384 | GTGGAAGCCTGATCCCATAC (SEQ ID NO: 384) | PE3b ngRNA spacer (AG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 965 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCTGATC | |
| | CCATACAGAAAGCT (SEQ ID NO: 965) | |
| 966 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCTGATC | |
| | CCATACAGAAAGCTTC (SEQ ID NO: 966) | |
| 967 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCT (SEQ ID NO: 967) | |
| 968 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCTGATC | |
| | CCATACAGAAAGCTTCTG (SEQ ID NO: 968) | |
| 969 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCTGATC | |
| | CCATACAGAAAGCTTTTT (SEQ ID NO: 969) | |
| 970 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTC (SEQ ID NO: 970) | |
| 971 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCT (SEQ ID NO: 971) | |
| 972 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCTGATC | |
| | CCATACAGAAAGCTTCTGCA (SEQ ID NO: 972) | |
| 973 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCTGATC | |
| | CCATACAGAAAGCTTCTTTT (SEQ ID NO: 973) | |
| 974 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTCTG (SEQ ID NO: 974) | |
| 975 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTC (SEQ ID NO: 975) | |
| 976 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCT (SEQ ID NO: 976) | |
| 977 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTTTT (SEQ ID NO: 977) | |
| 978 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCTGATC | |
| | CCATACAGAAAGCTTCTGTTTT (SEQ ID NO: 978) | |
| 979 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTCTGCA (SEQ ID NO: | |
| | 979) | |
| 980 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTCTG (SEQ ID NO: | |
| | 980) | |
| 981 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTC (SEQ ID NO: | |
| | 981) | |
| 982 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCT (SEQ ID NO: | |
| | 982) | |
| 983 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTCTTTT (SEQ ID | |
| | NO: 983) | |
| 984 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTTTT (SEQ ID | |
| | NO: 984) | |
| 985 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCTGATC | |
| | CCATACAGAAAGCTTCTGCATTTT (SEQ ID NO: | |
| | 985) | |
| 986 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTCTGCA (SEQ ID NO: | |
| | 986) | |
| 987 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTCTG (SEQ ID NO: | |
| | 987) | |
| 988 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTC (SEQ ID NO: | |
| | 988) | |
| 989 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTCTGTTTT (SEQ ID NO: | |
| | 989) | |
| 990 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTCTTTT (SEQ ID NO: | |
| | 990) | |
| 991 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTTTT (SEQ ID NO: | |
| | 991) | |
| 992 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTCTGCA (SEQ ID | |
| | NO: 992) | |
| 993 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTCTG (SEQ ID | |
| | NO: 993) | |
| 994 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGCCTG | |
| | ATCCCATACAGAAAGCTTCTGCATTTT (SEQ ID | |
| | NO: 994) | |
| 995 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTCTGTTTT (SEQ ID | |
| | NO: 995) | |
| 996 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTCTTTT (SEQ ID | |
| | NO: 996) | |
| 997 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTTTT (SEQ ID | |
| | NO: 997) | |
| 998 | GGATGCAGAAGCTTTCTGTAGTTTTAGAGCTAGA | pegRNA |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTCTGCA (SEQ | |
| | ID NO: 998) | |
| 999 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCAAGCC | |
| | TGATCCCATACAGAAAGCTTCTGCATTTT (SEQ ID | |
| | NO: 999) | |
| 1000 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTCTGTTTT (SEQ ID | |
| | NO: 1000) | |
| 1001 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTCTTTT (SEQ ID | |
| | NO: 1001) | |
| 1002 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGGAAG | |
| | CCTGATCCCATACAGAAAGCTTCTGCATTTT (SEQ | |
| | ID NO: 1002) | |
| 1003 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTCTGTTTT | |
| | (SEQ ID NO: 1003) | |
| 1004 | GGATGCAGAAGCTTTCTGTAGTTTTAGAGCTAGA | pegRNA, contains 3′ TTTT |
| | AATAGCAAGTTAAAATAAGGCTAGTCCGTTATCA | |
| | ACTTGAAAAAGTGGCACCGAGTCGGTGCGTGGA | |
| | AGCCTGATCCCATACAGAAAGCTTCTGCATTTT | |
| | (SEQ ID NO: 1004) | |
| 1005 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCTGATC | TTTT |
| | CCATACAGAAAGCTAACATTGACGCGTCTCTACG | |
| | TGGGGGCGCGTTTT (SEQ ID NO: 1005) | |
| 1006 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCTGATC | TTTT |
| | CCATACAGAAAGCTTCAACATTGACGCGTCTCTA | |
| | CGTGGGGGCGCGTTTT (SEQ ID NO: 1006) | |
| 1007 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGCCTG | TTTT |
| | ATCCCATACAGAAAGCTAACATTGACGCGTCTCT | |
| | ACGTGGGGGCGCGTTTT (SEQ ID NO: 1007) | |
| 1008 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCTGATC | TTTT |
| | CCATACAGAAAGCTTCTGAACATTGACGCGTCTC | |
| | TACGTGGGGGCGCGTTTT (SEQ ID NO: 1008) | |
| 1009 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGCCTG | TTTT |
| | ATCCCATACAGAAAGCTTCAACATTGACGCGTCT | |
| | CTACGTGGGGGCGCGTTTT (SEQ ID NO: 1009) | |
| 1010 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCAAGCC | TTTT |
| | TGATCCCATACAGAAAGCTAACATTGACGCGTCT | |
| | CTACGTGGGGGCGCGTTTT (SEQ ID NO: 1010) | |
| 1011 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCTGATC | TTTT |
| | CCATACAGAAAGCTTCTGCAAACATTGACGCGTC | |
| | TCTACGTGGGGGCGCGTTTT (SEQ ID NO: 1011) | |
| 1012 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGCCTG | TTTT |
| | ATCCCATACAGAAAGCTTCTGAACATTGACGCGT | |
| | CTCTACGTGGGGGCGCGTTTT (SEQ ID NO: 1012) | |
| 1013 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCAAGCC | TTTT |
| | TGATCCCATACAGAAAGCTTCAACATTGACGCGT | |
| | CTCTACGTGGGGGCGCGTTTT (SEQ ID NO: 1013) | |
| 1014 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGGAAG | TTTT |
| | CCTGATCCCATACAGAAAGCTAACATTGACGCGT | |
| | CTCTACGTGGGGGCGCGTTTT (SEQ ID NO: 1014) | |
| 1015 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGCCTG | TTTT |
| | ATCCCATACAGAAAGCTTCTGCAAACATTGACGC | |
| | GTCTCTACGTGGGGGCGCGTTTT (SEQ ID NO: | |
| | 1015) | |
| 1016 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCAAGCC | TTTT |
| | TGATCCCATACAGAAAGCTTCTGAACATTGACGC | |
| | GTCTCTACGTGGGGGCGCGTTTT (SEQ ID NO: | |
| | 1016) | |
| 1017 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGGAAG | TTTT |
| | CCTGATCCCATACAGAAAGCTTCAACATTGACGC | |
| | GTCTCTACGTGGGGGCGCGTTTT (SEQ ID NO: | |
| | 1017) | |
| 1018 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGTGGA | TTTT |
| | AGCCTGATCCCATACAGAAAGCTAACATTGACG | |
| | CGTCTCTACGTGGGGGCGCGTTTT (SEQ ID NO: | |
| | 1018) | |
| 1019 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCAAGCC | TTTT |
| | TGATCCCATACAGAAAGCTTCTGCAAACATTGAC | |
| | GCGTCTCTACGTGGGGGCGCGTTTT (SEQ ID NO: | |
| | 1019) | |
| 1020 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGGAAG | TTTT |
| | CCTGATCCCATACAGAAAGCTTCTGAACATTGAC | |
| | GCGTCTCTACGTGGGGGCGCGTTTT (SEQ ID NO: | |
| | 1020) | |
| 1021 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGTGGA | TTTT |
| | AGCCTGATCCCATACAGAAAGCTTCAACATTGAC | |
| | GCGTCTCTACGTGGGGGCGCGTTTT (SEQ ID NO: | |
| | 1021) | |
| 1022 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGGAAG | TTTT |
| | CCTGATCCCATACAGAAAGCTTCTGCAAACATTG | |
| | ACGCGTCTCTACGTGGGGGCGCGTTTT (SEQ ID | |
| | NO: 1022) | |
| 1023 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGTGGA | TTTT |
| | AGCCTGATCCCATACAGAAAGCTTCTGAACATTG | |
| | ACGCGTCTCTACGTGGGGGCGCGTTTT (SEQ ID | |
| | NO: 1023) | |
| 1024 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591; 3′ |
| | ACTTGAAAAAGTGGCACCGAGTCGGTGCGTGGA | TTTT |
| | AGCCTGATCCCATACAGAAAGCTTCTGCAAACAT | |
| | TGACGCGTCTCTACGTGGGGGCGCGTTTT (SEQ | |
| | ID NO: 1024) | |
| 323 | GTCTTGCTGGGTTTCCAAAGGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG; GGG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG; AGG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG; TGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG; GGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG; AGG PAM), contains 3′ |
| | AAATAGCAAGTTAAAATAAGGCTAGTCCGTTATC | TTTT |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG; TGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG; TGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG; AGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG; AGG PAM), contains 3′ |
| | AAATAGCAAGTTAAAATAAGGCTAGTCCGTTATC | TTTT, 5′G |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG; GGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG; TGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG; TGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 343) | |
| 3611 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTGATC | |
| | CCATACAGAAAGCTAACATTGACGCGTCTCTACG | |
| | TGGGGGCGCG (SEQ ID NO: 3611) | |
| 3612 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTGATC | |
| | CCATACAGAAAGCTTCAACATTGACGCGTCTCTA | |
| | CGTGGGGGCGCG (SEQ ID NO: 3612) | |
| 3613 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGCCTG | |
| | ATCCCATACAGAAAGCTAACATTGACGCGTCTCT | |
| | ACGTGGGGGCGCG (SEQ ID NO: 3613) | |
| 3614 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTGATC | |
| | CCATACAGAAAGCTTCTGAACATTGACGCGTCTC | |
| | TACGTGGGGGCGCG (SEQ ID NO: 3614) | |
| 3615 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGCCTG | |
| | ATCCCATACAGAAAGCTTCAACATTGACGCGTCT | |
| | CTACGTGGGGGCGCG (SEQ ID NO: 3615) | |
| 3616 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCAAGCC | |
| | TGATCCCATACAGAAAGCTAACATTGACGCGTCT | |
| | CTACGTGGGGGCGCG (SEQ ID NO: 3616) | |
| 3617 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTGATC | |
| | CCATACAGAAAGCTTCTGCAAACATTGACGCGTC | |
| | TCTACGTGGGGGCGCG (SEQ ID NO: 3617) | |
| 3618 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGCCTG | |
| | ATCCCATACAGAAAGCTTCTGAACATTGACGCGT | |
| | CTCTACGTGGGGGCGCG (SEQ ID NO: 3618) | |
| 3619 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCAAGCC | |
| | TGATCCCATACAGAAAGCTTCAACATTGACGCGT | |
| | CTCTACGTGGGGGCGCG (SEQ ID NO: 3619) | |
| 3620 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGGAAG | |
| | CCTGATCCCATACAGAAAGCTAACATTGACGCGT | |
| | CTCTACGTGGGGGCGCG (SEQ ID NO: 3620) | |
| 3621 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGCCTG | |
| | ATCCCATACAGAAAGCTTCTGCAAACATTGACGC | |
| | GTCTCTACGTGGGGGCGCG (SEQ ID NO: 3621) | |
| 3622 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCAAGCC | |
| | TGATCCCATACAGAAAGCTTCTGAACATTGACGC | |
| | GTCTCTACGTGGGGGCGCG (SEQ ID NO: 3622) | |
| 3623 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGGAAG | |
| | CCTGATCCCATACAGAAAGCTTCAACATTGACGC | |
| | GTCTCTACGTGGGGGCGCG (SEQ ID NO: 3623) | |
| 3624 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTGGA | |
| | AGCCTGATCCCATACAGAAAGCTAACATTGACG | |
| | CGTCTCTACGTGGGGGCGCG (SEQ ID NO: 3624) | |
| 3625 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCAAGCC | |
| | TGATCCCATACAGAAAGCTTCTGCAAACATTGAC | |
| | GCGTCTCTACGTGGGGGCGCG (SEQ ID NO: 3625) | |
| 3626 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGGAAG | |
| | CCTGATCCCATACAGAAAGCTTCTGAACATTGAC | |
| | GCGTCTCTACGTGGGGGCGCG (SEQ ID NO: 3626) | |
| 3627 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTGGA | |
| | AGCCTGATCCCATACAGAAAGCTTCAACATTGAC | |
| | GCGTCTCTACGTGGGGGCGCG (SEQ ID NO: 3627) | |
| 3628 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGGAAG | |
| | CCTGATCCCATACAGAAAGCTTCTGCAAACATTG | |
| | ACGCGTCTCTACGTGGGGGCGCG (SEQ ID NO: | |
| | 3628) | |
| 3629 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTGGA | |
| | AGCCTGATCCCATACAGAAAGCTTCTGAACATTG | |
| | ACGCGTCTCTACGTGGGGGCGCG (SEQ ID NO: | |
| | 3629) | |
| 3630 | GGATGCAGAAGCTTTCTGTAGTTTTAGAGCTAGA | pegRNA, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTGGA | |
| | AGCCTGATCCCATACAGAAAGCTTCTGCAAACAT | |
| | TGACGCGTCTCTACGTGGGGGCGCG (SEQ ID NO: | |
| | 3630) | |
| 3604 | GTCTTGCTGGGTTTCCAAAGGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3604) | |
| 3605 | GTGAAGACAGGTGTCTTGCTGTTTTAGAGCTAGA | PE3 ngRNA (GG; GGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3605) | |
| 3606 | GAACTGAGCCACGTGAAGACGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591 |
| | TTGACGCGTCTCTACGTGGGGGCGCG (SEQ ID | |
| | NO: 3606) | |
| 3607 | CGTGAAGACAGGTGTCTTGCGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3607) | |
| 3608 | ACATCTTCCTTAGGAACTCCGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3608) | |
| 3609 | AGGCTTCATACATCTTCCTTGTTTTAGAGCTAGA | PE3 ngRNA (AG; AGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3609) | |
| 3610 | ACTACTTACCATCTCTTTCAGTTTTAGAGCTAGA | PE3 ngRNA (AG; AGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591 |
| | TGACGCGTCTCTACGTGGGGGCGCG (SEQ ID NO: | |
| | 3610) | |

TABLE 16

| Sequence | | |
|---|---|---|
| Number | Sequence | Description |
| 1025 | TTTCCAAAGTGGAAGCC (SEQ ID NO: 1025) | PEgRNA spacer (TG; TGA PAM) |
| 1026 | GTTTCCAAAGTGGAAGCC (SEQ ID NO: 1026) | PEgRNA spacer (TG; TGA PAM) |
| 1027 | GGTTTCCAAAGTGGAAGCC (SEQ ID NO: 1027) | PEgRNA spacer (TG; TGA PAM) |
| 211 | GGGTTTCCAAAGTGGAAGCC (SEQ ID NO: 211) | PEgRNA spacer (TG; TGA PAM) |
| 1028 | TGGGTTTCCAAAGTGGAAGCC (SEQ ID NO: 1028) | PEgRNA spacer (TG; TGA PAM) |
| 1029 | CTGGGTTTCCAAAGTGGAAGCC (SEQ ID NO: | PEgRNA spacer (TG; TGA PAM) |
| | 1029) | |
| 1030 | TTCCA | PBS |
| 1031 | TTCCAC | PBS |
| 1032 | TTCCACT | PBS |
| 1033 | TTCCACTT | PBS |
| 1034 | TTCCACTTT | PBS |
| 1035 | TTCCACTTTG (SEQ ID NO: 1035) | PBS |
| 1036 | TTCCACTTTGG (SEQ ID NO: 1036) | PBS |
| 1037 | TTCCACTTTGGA (SEQ ID NO: 1037) | PBS |
| 1038 | TTCCACTTTGGAA (SEQ ID NO: 1038) | PBS |
| 1039 | TTCCACTTTGGAAA (SEQ ID NO: 1039) | PBS |
| 1040 | TTCCACTTTGGAAAC (SEQ ID NO: 1040) | PBS |
| 1041 | TTCCACTTTGGAAACC (SEQ ID NO: 1041) | PBS |
| 1042 | TTCCACTTTGGAAACCC (SEQ ID NO: 1042) | PBS |
| 1043 | TTCCACTTTGGAAACCCA (SEQ ID NO: 1043) | PBS |
| 1044 | TTCCACTTTGGAAACCCAG (SEQ ID NO: 1044) | PBS |
| 1045 | GGGACCAGGC (SEQ ID NO: 1045) | RTT (TGA-to-TGG PAM silencing edit) |
| 1046 | GGGATCAGGC (SEQ ID NO: 1046) | RTT*1, 2 |
| 1047 | TGGGACCAGGC (SEQ ID NO: 1047) | RTT (TGA-to-TGG PAM silencing edit) |
| 1048 | TGGGATCAGGC (SEQ ID NO: 1048) | RTT*1, 2 |
| 1049 | ATGGGACCAGGC (SEQ ID NO: 1049) | RTT (TGA-to-TGG PAM silencing edit) |
| 1050 | ATGGGATCAGGC (SEQ ID NO: 1050) | RTT*1, 2 |
| 1051 | TATGGGACCAGGC (SEQ ID NO: 1051) | RTT (TGA-to-TGG PAM silencing edit) |
| 1052 | TATGGGATCAGGC (SEQ ID NO: 1052) | RTT*1, 2 |
| 1053 | GTATGGGACCAGGC (SEQ ID NO: 1053) | RTT (TGA-to-TGG PAM silencing edit) |
| 1054 | GTATGGGATCAGGC (SEQ ID NO: 1054) | RTT*1, 2 |
| 1055 | TGTATGGGACCAGGC (SEQ ID NO: 1055) | RTT (TGA-to-TGG PAM silencing edit) |
| 1056 | TGTATGGGATCAGGC (SEQ ID NO: 1056) | RTT*1, 2 |
| 1057 | CTGTATGGGACCAGGC (SEQ ID NO: 1057) | RTT (TGA-to-TGG PAM silencing edit) |
| 1058 | CTGTATGGGATCAGGC (SEQ ID NO: 1058) | RTT*1, 2 |
| 1059 | TCTGTATGGGACCAGGC (SEQ ID NO: 1059) | RTT (TGA-to-TGG PAM silencing edit) |
| 1060 | TCTGTATGGGATCAGGC (SEQ ID NO: 1060) | RTT*1, 2 |
| 1061 | TTCTGTATGGGACCAGGC (SEQ ID NO: 1061) | RTT (TGA-to-TGG PAM silencing edit) |
| 1062 | TTCTGTATGGGATCAGGC (SEQ ID NO: 1062) | RTT*1, 2 |
| 1063 | TTTCTGTATGGGACCAGGC (SEQ ID NO: 1063) | RTT (TGA-to-TGG PAM silencing edit) |
| 1064 | TTTCTGTATGGGATCAGGC (SEQ ID NO: 1064) | RTT*1, 2 |
| 1065 | CTTTCTGTATGGGACCAGGC (SEQ ID NO: 1065) | RTT (TGA-to-TGG PAM silencing edit) |
| 1066 | CTTTCTGTATGGGATCAGGC (SEQ ID NO: 1066) | RTT*1, 2 |
| 1067 | GCTTTCTGTATGGGACCAGGC (SEQ ID NO: 1067) | RTT (TGA-to-TGG PAM silencing edit) |
| 1068 | GCTTTCTGTATGGGATCAGGC (SEQ ID NO: 1068) | RTT*1, 2 |
| 1069 | AGCTTTCTGTATGGGACCAGGC (SEQ ID NO: 1069) | RTT (TGA-to-TGG PAM silencing edit) |
| 1070 | AGCTTTCTGTATGGGATCAGGC (SEQ ID NO: 1070) | RTT*1, 2 |
| 1071 | AAGCTTTCTGTATGGGACCAGGC (SEQ ID NO: | RTT (TGA-to-TGG PAM silencing edit) |
| | 1071) | |
| 1072 | AAGCTTTCTGTATGGGATCAGGC (SEQ ID NO: | RTT*1, 2 |
| | 1072) | |
| 1073 | GAAGCTTTCTGTATGGGACCAGGC (SEQ ID NO: | RTT (TGA-to-TGG PAM silencing edit) |
| | 1073) | |
| 1074 | GAAGCTTTCTGTATGGGATCAGGC (SEQ ID NO: | RTT*1, 2 |
| | 1074) | |
| 1075 | AGAAGCTTTCTGTATGGGACCAGGC (SEQ ID NO: | RTT (TGA-to-TGG PAM silencing edit) |
| | 1075) | |
| 1076 | AGAAGCTTTCTGTATGGGATCAGGC (SEQ ID NO: | RTT*1, 2 |
| | 1076) | |
| 1077 | CAGAAGCTTTCTGTATGGGACCAGGC (SEQ ID | RTT (TGA-to-TGG PAM silencing edit) |
| | NO: 1077) | |
| 1078 | CAGAAGCTTTCTGTATGGGATCAGGC (SEQ ID | RTT*1, 2 |
| | NO: 1078) | |
| 1079 | GCAGAAGCTTTCTGTATGGGACCAGGC (SEQ ID | RTT (TGA-to-TGG PAM silencing edit) |
| | NO: 1079) | |
| 1080 | GCAGAAGCTTTCTGTATGGGATCAGGC (SEQ ID | RTT*1, 2 |
| | NO: 1080) | |
| 1081 | TGCAGAAGCTTTCTGTATGGGACCAGGC (SEQ ID | RTT (TGA-to-TGG PAM silencing edit) |
| | NO: 1081) | |
| 1082 | TGCAGAAGCTTTCTGTATGGGATCAGGC (SEQ ID | RTT*1, 2 |
| | NO: 1082) | |
| 1083 | ATGCAGAAGCTTTCTGTATGGGACCAGGC (SEQ | RTT (TGA-to-TGG PAM silencing edit) |
| | ID NO: 1083) | |
| 1084 | ATGCAGAAGCTTTCTGTATGGGATCAGGC (SEQ | RTT*1, 2 |
| | ID NO: 1084) | |
| 1085 | GATGCAGAAGCTTTCTGTATGGGACCAGGC (SEQ | RTT (TGA-to-TGG PAM silencing edit) |
| | ID NO: 1085) | |
| 1086 | GATGCAGAAGCTTTCTGTATGGGATCAGGC (SEQ | RTT*1, 2 |
| | ID NO: 1086) | |
| 1087 | GGATGCAGAAGCTTTCTGTATGGGACCAGGC | RTT (TGA-to-TGG PAM silencing edit) |
| | (SEQ ID NO: 1087) | |
| 1088 | GGATGCAGAAGCTTTCTGTATGGGATCAGGC | RTT*1, 2 |
| | (SEQ ID NO: 1088) | |
| 1089 | TGGATGCAGAAGCTTTCTGTATGGGACCAGGC | RTT (TGA-to-TGG PAM silencing edit) |
| | (SEQ ID NO: 1089) | |
| 1090 | TGGATGCAGAAGCTTTCTGTATGGGATCAGGC | RTT*1, 2 |
| | (SEQ ID NO: 1090) | |
| 1091 | TTGGATGCAGAAGCTTTCTGTATGGGACCAGGC | RTT (TGA-to-TGG PAM silencing edit) |
| | (SEQ ID NO: 1091) | |
| 1092 | TTGGATGCAGAAGCTTTCTGTATGGGATCAGGC | RTT*1, 2 |
| | (SEQ ID NO: 1092) | |
| 1093 | TTTGGATGCAGAAGCTTTCTGTATGGGACCAGGC | RTT (TGA-to-TGG PAM silencing edit) |
| | (SEQ ID NO: 1093) | |
| 1094 | TTTGGATGCAGAAGCTTTCTGTATGGGATCAGGC | RTT*1, 2 |
| | (SEQ ID NO: 1094) | |
| 1095 | TTTTGGATGCAGAAGCTTTCTGTATGGGACCAGG | RTT (TGA-to-TGG PAM silencing edit) |
| | C (SEQ ID NO: 1095) | |
| 1096 | TTTTGGATGCAGAAGCTTTCTGTATGGGATCAGG | RTT*1, 2 |
| | C (SEQ ID NO: 1096) | |
| 1097 | GTTTTGGATGCAGAAGCTTTCTGTATGGGACCAG | RTT (TGA-to-TGG PAM silencing edit) |
| | GC (SEQ ID NO: 1097) | |
| 1098 | GTTTTGGATGCAGAAGCTTTCTGTATGGGATCAG | RTT*1, 2 |
| | GC (SEQ ID NO: 1098) | |
| 1099 | AGTTTTGGATGCAGAAGCTTTCTGTATGGGACCA | RTT (TGA-to-TGG PAM silencing edit) |
| | GGC (SEQ ID NO: 1099) | |
| 1100 | AGTTTTGGATGCAGAAGCTTTCTGTATGGGATCA | RTT*1, 2 |
| | GGC (SEQ ID NO: 1100) | |
| 1101 | CAGTTTTGGATGCAGAAGCTTTCTGTATGGGACC | RTT (TGA-to-TGG PAM silencing edit) |
| | AGGC (SEQ ID NO: 1101) | |
| 1102 | CAGTTTTGGATGCAGAAGCTTTCTGTATGGGATC | RTT*1, 2 |
| | AGGC (SEQ ID NO: 1102) | |
| 1103 | TCAGTTTTGGATGCAGAAGCTTTCTGTATGGGAC | RTT (TGA-to-TGG PAM silencing edit) |
| | CAGGC (SEQ ID NO: 1103) | |
| 1104 | TCAGTTTTGGATGCAGAAGCTTTCTGTATGGGAT | RTT*1, 2 |
| | CAGGC (SEQ ID NO: 1104) | |
| 1105 | ATCAGTTTTGGATGCAGAAGCTTTCTGTATGGGA | RTT (TGA-to-TGG PAM silencing edit) |
| | CCAGGC (SEQ ID NO: 1105) | |
| 1106 | ATCAGTTTTGGATGCAGAAGCTTTCTGTATGGGA | RTT*1, 2 |
| | TCAGGC (SEQ ID NO: 1106) | |
| 24 | AAGATGGCTCAAGATTCAGT (SEQ ID NO: 24) | PE3 ngRNA spacer (AG; AGA PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b*1 ngRNA spacer (GG PAM) |
| 27 | AGATCTTTCTTGTGATTATC (SEQ ID NO: 27) | PE3 ngRNA spacer (AG PAM) |
| 1107 | ATTTCCTTCAGTGCTGGACA (SEQ ID NO: 1107) | PE3 ngRNA spacer (GG PAM) |
| 1108 | CAGTGCTGGACAGGCTGCTG (SEQ ID NO: 1108) | PE3 ngRNA spacer (TG; TGA PAM) |
| 1109 | CATTTCCTTCAGTGCTGGAC (SEQ ID NO: 1109) | PE3 ngRNA spacer (AG PAM) |
| 29 | CCTTTTCGCTTTTTCCAAGA (SEQ ID NO: 29) | PE3 ngRNA spacer (TG PAM) |
| 1110 | CTGGACAGGCTGCTGTGAAG (SEQ ID NO: 1110) | PE3 ngRNA spacer (GG; GGA PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b*2 ngRNA spacer (AG PAM) |
| 32 | GATGCAGAAGCTTTCTGTAT (SEQ ID NO: 32) | PE3 ngRNA spacer (GG; GGA PAM) |
| 33 | GATTCAGTAGATCTTTCTTG (SEQ ID NO: 33) | PE3 ngRNA spacer (TG; TGA PAM) |
| 1111 | GCTGGACAGGCTGCTGTGAA (SEQ ID NO: 1111) | PE3 ngRNA spacer (GG PAM) |
| 34 | GCTTTTTCCAAGATGGCTCA (SEQ ID NO: 34) | PE3 ngRNA spacer (AG; AGA PAM) |
| 35 | GGATGCAGAAGCTTTCTGTA (SEQ ID NO: 35) | PE3 ngRNA spacer (TG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG; AGA PAM) |
| 39 | TCCAAGATGGCTCAAGATTC (SEQ ID NO: 39) | PE3 ngRNA spacer (AG PAM) |
| 1112 | TCCTTCAGTGCTGGACAGGC (SEQ ID NO: 1112) | PE3 ngRNA spacer (TG PAM) |
| 41 | TGATTATCAGTTTTGGATGC (SEQ ID NO: 41) | PE3 ngRNA spacer (AG; AGA PAM) |
| 1113 | TGCTGGACAGGCTGCTGTGA (SEQ ID NO: 1113) | PE3 ngRNA spacer (AG PAM) |
| 44 | TTATCAGTTTTGGATGCAGA (SEQ ID NO: 44) | PE3 ngRNA spacer (AG PAM) |
| 1114 | TTCAGTGCTGGACAGGCTGC (SEQ ID NO: 1114) | PE3 ngRNA spacer (TG PAM) |
| 45 | TTCTTGTGATTATCAGTTTT (SEQ ID NO: 45) | PE3 ngRNA spacer (GG; GGA PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b*2 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b*2 ngRNA (AG PAM), end adaptations |
| | AATAGCAAGTTAAAATAAGGCTAGTCCGTTATCA | |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b*2 ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |

TABLE 17

| Sequence | | |
|---|---|---|
| Number | Sequence | Description |
| 1115 | GTCTTGCTGGGTTTCCA (SEQ ID NO: 1115) | PEgRNA spacer (AAG PAM) |
| 1116 | TGTCTTGCTGGGTTTCCA (SEQ ID NO: 1116) | PEgRNA spacer (AAG PAM) |
| 1117 | GTGTCTTGCTGGGTTTCCA (SEQ ID NO: 1117) | PEgRNA spacer (AAG PAM) |
| 486 | GGTGTCTTGCTGGGTTTCCA (SEQ ID NO: 486) | PEgRNA spacer (AAG PAM) |
| 1118 | AGGTGTCTTGCTGGGTTTCCA (SEQ ID NO: 1118) | PEgRNA spacer (AAG PAM) |
| 1119 | CAGGTGTCTTGCTGGGTTTCCA (SEQ ID NO: 1119) | PEgRNA spacer (AAG PAM) |
| 1120 | AAACC | PBS |
| 1121 | AAACCC | PBS |
| 1122 | AAACCCA | PBS |
| 1123 | AAACCCAG | PBS |
| 1124 | AAACCCAGC | PBS |
| 1125 | AAACCCAGCA (SEQ ID NO: 1125) | PBS |
| 1126 | AAACCCAGCAA (SEQ ID NO: 1126) | PBS |
| 1127 | AAACCCAGCAAG (SEQ ID NO: 1127) | PBS |
| 1128 | AAACCCAGCAAGA (SEQ ID NO: 1128) | PBS |
| 1129 | AAACCCAGCAAGAC (SEQ ID NO: 1129) | PBS |
| 1130 | AAACCCAGCAAGACA (SEQ ID NO: 1130) | PBS |
| 1131 | AAACCCAGCAAGACAC (SEQ ID NO: 1131) | PBS |
| 1132 | AAACCCAGCAAGACACC (SEQ ID NO: 1132) | PBS |
| 1133 | AAACCCAGCAAGACACCT (SEQ ID NO: 1133) | PBS |
| 1134 | AAACCCAGCAAGACACCTG (SEQ ID NO: 1134) | PBS |
| 1135 | GATCAGGCTTCCACATTGG (SEQ ID NO: 1135) | RTT (AAG-to-ATG PAM silencing edit) |
| 1136 | GATCAGGCTTCCACCTTGG (SEQ ID NO: 1136) | RTT (AAG-to-AGG PAM silencing edit) |
| 1137 | GATCAGGCTTCCACGTTGG (SEQ ID NO: 1137) | RTT (AAG-to-ACG PAM silencing edit) |
| 1138 | GATCAGGCTTCCACTTTGG (SEQ ID NO: 1138) | RTT |
| 1139 | GGATCAGGCTTCCACATTGG (SEQ ID NO: 1139) | RTT (AAG-to-ATG PAM silencing edit) |
| 1140 | GGATCAGGCTTCCACCTTGG (SEQ ID NO: 1140) | RTT (AAG-to-AGG PAM silencing edit) |
| 1141 | GGATCAGGCTTCCACGTTGG (SEQ ID NO: 1141) | RTT (AAG-to-ACG PAM silencing edit) |
| 1142 | GGATCAGGCTTCCACTTTGG (SEQ ID NO: 1142) | RTT |
| 1143 | GGGATCAGGCTTCCACATTGG (SEQ ID NO: 1143) | RTT (AAG-to-ATG PAM silencing edit) |
| 1144 | GGGATCAGGCTTCCACCTTGG (SEQ ID NO: 1144) | RTT (AAG-to-AGG PAM silencing edit) |
| 1145 | GGGATCAGGCTTCCACGTTGG (SEQ ID NO: 1145) | RTT (AAG-to-ACG PAM silencing edit) |
| 1146 | GGGATCAGGCTTCCACTTTGG (SEQ ID NO: 1146) | RTT |
| 1147 | TGGGATCAGGCTTCCACATTGG (SEQ ID NO: 1147) | RTT (AAG-to-ATG PAM silencing edit) |
| 1148 | TGGGATCAGGCTTCCACCTTGG (SEQ ID NO: 1148) | RTT (AAG-to-AGG PAM silencing edit) |
| 1149 | TGGGATCAGGCTTCCACGTTGG (SEQ ID NO: 1149) | RTT (AAG-to-ACG PAM silencing edit) |
| 1150 | TGGGATCAGGCTTCCACTTTGG (SEQ ID NO: 1150) | RTT |
| 1151 | ATGGGATCAGGCTTCCACATTGG (SEQ ID NO: | RTT (AAG-to-ATG PAM silencing edit) |
| | 1151) | |
| 1152 | ATGGGATCAGGCTTCCACCTTGG (SEQ ID NO: | RTT (AAG-to-AGG PAM silencing edit) |
| | 1152) | |
| 1153 | ATGGGATCAGGCTTCCACGTTGG (SEQ ID NO: | RTT (AAG-to-ACG PAM silencing edit) |
| | 1153) | |
| 1154 | ATGGGATCAGGCTTCCACTTTGG (SEQ ID NO: | RTT |
| | 1154) | |
| 1155 | TATGGGATCAGGCTTCCACATTGG (SEQ ID NO: | RTT (AAG-to-ATG PAM silencing edit) |
| | 1155) | |
| 1156 | TATGGGATCAGGCTTCCACCTTGG (SEQ ID NO: | RTT (AAG-to-AGG PAM silencing edit) |
| | 1156) | |
| 1157 | TATGGGATCAGGCTTCCACGTTGG (SEQ ID NO: | RTT (AAG-to-ACG PAM silencing edit) |
| | 1157) | |
| 1158 | TATGGGATCAGGCTTCCACTTTGG (SEQ ID NO: | RTT |
| | 1158) | |
| 1159 | GTATGGGATCAGGCTTCCACATTGG (SEQ ID NO: | RTT (AAG-to-ATG PAM silencing edit) |
| | 1159) | |
| 1160 | GTATGGGATCAGGCTTCCACCTTGG (SEQ ID NO: | RTT (AAG-to-AGG PAM silencing edit) |
| | 1160) | |
| 1161 | GTATGGGATCAGGCTTCCACGTTGG (SEQ ID NO: | RTT (AAG-to-ACG PAM silencing edit) |
| | 1161) | |
| 1162 | GTATGGGATCAGGCTTCCACTTTGG (SEQ ID NO: | RTT |
| | 1162) | |
| 1163 | TGTATGGGATCAGGCTTCCACATTGG (SEQ ID | RTT (AAG-to-ATG PAM silencing edit) |
| | NO: 1163) | |
| 1164 | TGTATGGGATCAGGCTTCCACCTTGG (SEQ ID | RTT (AAG-to-AGG PAM silencing edit) |
| | NO: 1164) | |
| 1165 | TGTATGGGATCAGGCTTCCACGTTGG (SEQ ID | RTT (AAG-to-ACG PAM silencing edit) |
| | NO: 1165) | |
| 1166 | TGTATGGGATCAGGCTTCCACTTTGG (SEQ ID NO: | RTT |
| | 1166) | |
| 1167 | CTGTATGGGATCAGGCTTCCACATTGG (SEQ ID | RTT (AAG-to-ATG PAM silencing edit) |
| | NO: 1167) | |
| 1168 | CTGTATGGGATCAGGCTTCCACCTTGG (SEQ ID | RTT (AAG-to-AGG PAM silencing edit) |
| | NO: 1168) | |
| 1169 | CTGTATGGGATCAGGCTTCCACGTTGG (SEQ ID | RTT (AAG-to-ACG PAM silencing edit) |
| | NO: 1169) | |
| 1170 | CTGTATGGGATCAGGCTTCCACTTTGG (SEQ ID | RTT |
| | NO: 1170) | |
| 1171 | TCTGTATGGGATCAGGCTTCCACATTGG (SEQ ID | RTT (AAG-to-ATG PAM silencing edit) |
| | NO: 1171) | |
| 1172 | TCTGTATGGGATCAGGCTTCCACCTTGG (SEQ ID | RTT (AAG-to-AGG PAM silencing edit) |
| | NO: 1172) | |
| 1173 | TCTGTATGGGATCAGGCTTCCACGTTGG (SEQ ID | RTT (AAG-to-ACG PAM silencing edit) |
| | NO: 1173) | |
| 1174 | TCTGTATGGGATCAGGCTTCCACTTTGG (SEQ ID | RTT |
| | NO: 1174) | |
| 1175 | TTCTGTATGGGATCAGGCTTCCACATTGG (SEQ ID | RTT (AAG-to-ATG PAM silencing edit) |
| | NO: 1175) | |
| 1176 | TTCTGTATGGGATCAGGCTTCCACCTTGG (SEQ ID | RTT (AAG-to-AGG PAM silencing edit) |
| | NO: 1176) | |
| 1177 | TTCTGTATGGGATCAGGCTTCCACGTTGG (SEQ ID | RTT (AAG-to-ACG PAM silencing edit) |
| | NO: 1177) | |
| 1178 | TTCTGTATGGGATCAGGCTTCCACTTTGG (SEQ ID | RTT |
| | NO: 1178) | |
| 1179 | TTTCTGTATGGGATCAGGCTTCCACATTGG (SEQ | RTT (AAG-to-ATG PAM silencing edit) |
| | ID NO: 1179) | |
| 1180 | TTTCTGTATGGGATCAGGCTTCCACCTTGG (SEQ | RTT (AAG-to-AGG PAM silencing edit) |
| | ID NO: 1180) | |
| 1181 | TTTCTGTATGGGATCAGGCTTCCACGTTGG (SEQ | RTT (AAG-to-ACG PAM silencing edit) |
| | ID NO: 1181) | |
| 1182 | TTTCTGTATGGGATCAGGCTTCCACTTTGG (SEQ | RTT |
| | ID NO: 1182) | |
| 1183 | CTTTCTGTATGGGATCAGGCTTCCACATTGG (SEQ | RTT (AAG-to-ATG PAM silencing edit) |
| | ID NO: 1183) | |
| 1184 | CTTTCTGTATGGGATCAGGCTTCCACCTTGG (SEQ | RTT (AAG-to-AGG PAM silencing edit) |
| | ID NO: 1184) | |
| 1185 | CTTTCTGTATGGGATCAGGCTTCCACGTTGG (SEQ | RTT (AAG-to-ACG PAM silencing edit) |
| | ID NO: 1185) | |
| 1186 | CTTTCTGTATGGGATCAGGCTTCCACTTTGG (SEQ | RTT |
| | ID NO: 1186) | |
| 1187 | GCTTTCTGTATGGGATCAGGCTTCCACATTGG | RTT (AAG-to-ATG PAM silencing edit) |
| | (SEQ ID NO: 1187) | |
| 1188 | GCTTTCTGTATGGGATCAGGCTTCCACCTTGG | RTT (AAG-to-AGG PAM silencing edit) |
| | (SEQ ID NO: 1188) | |
| 1189 | GCTTTCTGTATGGGATCAGGCTTCCACGTTGG | RTT (AAG-to-ACG PAM silencing edit) |
| | (SEQ ID NO: 1189) | |
| 1190 | GCTTTCTGTATGGGATCAGGCTTCCACTTTGG | RTT |
| | (SEQ ID NO: 1190) | |
| 1191 | AGCTTTCTGTATGGGATCAGGCTTCCACATTGG | RTT (AAG-to-ATG PAM silencing edit) |
| | (SEQ ID NO: 1191) | |
| 1192 | AGCTTTCTGTATGGGATCAGGCTTCCACCTTGG | RTT (AAG-to-AGG PAM silencing edit) |
| | (SEQ ID NO: 1192) | |
| 1193 | AGCTTTCTGTATGGGATCAGGCTTCCACGTTGG | RTT (AAG-to-ACG PAM silencing edit) |
| | (SEQ ID NO: 1193) | |
| 1194 | AGCTTTCTGTATGGGATCAGGCTTCCACTTTGG | RTT |
| | (SEQ ID NO: 1194) | |
| 1195 | AAGCTTTCTGTATGGGATCAGGCTTCCACATTGG | RTT (AAG-to-ATG PAM silencing edit) |
| | (SEQ ID NO: 1195) | |
| 1196 | AAGCTTTCTGTATGGGATCAGGCTTCCACCTTGG | RTT (AAG-to-AGG PAM silencing edit) |
| | (SEQ ID NO: 1196) | |
| 1197 | AAGCTTTCTGTATGGGATCAGGCTTCCACGTTGG | RTT (AAG-to-ACG PAM silencing edit) |
| | (SEQ ID NO: 1197) | |
| 1198 | AAGCTTTCTGTATGGGATCAGGCTTCCACTTTGG | RTT |
| | (SEQ ID NO: 1198) | |
| 1199 | GAAGCTTTCTGTATGGGATCAGGCTTCCACATTG | RTT (AAG-to-ATG PAM silencing edit) |
| | G (SEQ ID NO: 1199) | |
| 1200 | GAAGCTTTCTGTATGGGATCAGGCTTCCACCTTG | RTT (AAG-to-AGG PAM silencing edit) |
| | G (SEQ ID NO: 1200) | |
| 1201 | GAAGCTTTCTGTATGGGATCAGGCTTCCACGTTG | RTT (AAG-to-ACG PAM silencing edit) |
| | G (SEQ ID NO: 1201) | |
| 1202 | GAAGCTTTCTGTATGGGATCAGGCTTCCACTTTG | RTT |
| | G (SEQ ID NO: 1202) | |
| 1203 | AGAAGCTTTCTGTATGGGATCAGGCTTCCACATT | RTT (AAG-to-ATG PAM silencing edit) |
| | GG (SEQ ID NO: 1203) | |
| 1204 | AGAAGCTTTCTGTATGGGATCAGGCTTCCACCTT | RTT (AAG-to-AGG PAM silencing edit) |
| | GG (SEQ ID NO: 1204) | |
| 1205 | AGAAGCTTTCTGTATGGGATCAGGCTTCCACGTT | RTT (AAG-to-ACG PAM silencing edit) |
| | GG (SEQ ID NO: 1205) | |
| 1206 | AGAAGCTTTCTGTATGGGATCAGGCTTCCACTTT | RTT |
| | GG (SEQ ID NO: 1206) | |
| 1207 | CAGAAGCTTTCTGTATGGGATCAGGCTTCCACAT | RTT (AAG-to-ATG PAM silencing edit) |
| | TGG (SEQ ID NO: 1207) | |
| 1208 | CAGAAGCTTTCTGTATGGGATCAGGCTTCCACCT | RTT (AAG-to-AGG PAM silencing edit) |
| | TGG (SEQ ID NO: 1208) | |
| 1209 | CAGAAGCTTTCTGTATGGGATCAGGCTTCCACGT | RTT (AAG-to-ACG PAM silencing edit) |
| | TGG (SEQ ID NO: 1209) | |
| 1210 | CAGAAGCTTTCTGTATGGGATCAGGCTTCCACTT | RTT |
| | TGG (SEQ ID NO: 1210) | |
| 1211 | GCAGAAGCTTTCTGTATGGGATCAGGCTTCCACA | RTT (AAG-to-ATG PAM silencing edit) |
| | TTGG (SEQ ID NO: 1211) | |
| 1212 | GCAGAAGCTTTCTGTATGGGATCAGGCTTCCACC | RTT (AAG-to-AGG PAM silencing edit) |
| | TTGG (SEQ ID NO: 1212) | |
| 1213 | GCAGAAGCTTTCTGTATGGGATCAGGCTTCCACG | RTT (AAG-to-ACG PAM silencing edit) |
| | TTGG (SEQ ID NO: 1213) | |
| 1214 | GCAGAAGCTTTCTGTATGGGATCAGGCTTCCACT | RTT |
| | TTGG (SEQ ID NO: 1214) | |
| 1215 | TGCAGAAGCTTTCTGTATGGGATCAGGCTTCCAC | RTT (AAG-to-ATG PAM silencing edit) |
| | ATTGG (SEQ ID NO: 1215) | |
| 1216 | TGCAGAAGCTTTCTGTATGGGATCAGGCTTCCAC | RTT (AAG-to-AGG PAM silencing edit) |
| | CTTGG (SEQ ID NO: 1216) | |
| 1217 | TGCAGAAGCTTTCTGTATGGGATCAGGCTTCCAC | RTT (AAG-to-ACG PAM silencing edit) |
| | GTTGG (SEQ ID NO: 1217) | |
| 1218 | TGCAGAAGCTTTCTGTATGGGATCAGGCTTCCAC | RTT |
| | TTTGG (SEQ ID NO: 1218) | |
| 1219 | ATGCAGAAGCTTTCTGTATGGGATCAGGCTTCCA | RTT (AAG-to-ATG PAM silencing edit) |
| | CATTGG (SEQ ID NO: 1219) | |
| 1220 | ATGCAGAAGCTTTCTGTATGGGATCAGGCTTCCA | RTT (AAG-to-AGG PAM silencing edit) |
| | CCTTGG (SEQ ID NO: 1220) | |
| 1221 | ATGCAGAAGCTTTCTGTATGGGATCAGGCTTCCA | RTT (AAG-to-ACG PAM silencing edit) |
| | CGTTGG (SEQ ID NO: 1221) | |
| 1222 | ATGCAGAAGCTTTCTGTATGGGATCAGGCTTCCA | RTT |
| | CTTTGG (SEQ ID NO: 1222) | |
| 127 | AGAAGCTTTCTGTATGGGAT (SEQ ID NO: 127) | PE3b ngRNA spacer (CAG PAM) |
| 1223 | ATCACCTTTTCGCTTTTTCC (SEQ ID NO: 1223) | PE3 ngRNA spacer (AAG PAM) |
| 418 | ATTATCAGTTTTGGATGCAG (SEQ ID NO: 418) | PE3 ngRNA spacer (AAG PAM) |
| 1224 | CAAGATGGCTCAAGATTCAG (SEQ ID NO: 1224) | PE3 ngRNA spacer (TAG PAM) |
| 1225 | CGCTTTTTCCAAGATGGCTC (SEQ ID NO: 1225) | PE3 ngRNA spacer (AAG PAM) |
| 1226 | GTGATTATCAGTTTTGGATG (SEQ ID NO: 1226) | PE3 ngRNA spacer (CAG PAM) |
| 1227 | GTGCTGGACAGGCTGCTGTG (SEQ ID NO: 1227) | PE3 ngRNA spacer (AAG PAM) |
| 1228 | TAGATCTTTCTTGTGATTAT (SEQ ID NO: 1228) | PE3 ngRNA spacer (CAG PAM) |
| 1229 | TTCCAAGATGGCTCAAGATT (SEQ ID NO: 1229) | PE3 ngRNA spacer (CAG PAM) |

TABLE 18

| Sequence | | |
|---|---|---|
| Number | Sequence | Description |
| 1230 | TTGCTGGGTTTCCAAAG (SEQ ID NO: 1230) | PEgRNA spacer (TG; TGG PAM) |
| 1231 | CTTGCTGGGTTTCCAAAG (SEQ ID NO: 1231) | PERNA spacer (TG; TGG PAM) |
| 1232 | TCTTGCTGGGTTTCCAAAG (SEQ ID NO: 1232) | PERNA spacer (TG; TGG PAM) |
| 212 | GTCTTGCTGGGTTTCCAAAG (SEQ ID NO: 212) | PEgRNA spacer (TG; TGG PAM) |
| 1233 | TGTCTTGCTGGGTTTCCAAAG (SEQ ID NO: 1233) | PEgRNA spacer (TG; TGG PAM) |
| 1234 | GTGTCTTGCTGGGTTTCCAAAG | PEgRNA spacer (TG; TGG PAM) |
| | (SEQ ID NO: 1234) | |
| 1235 | TGGAA | PBS |
| 1236 | TGGAAA | PBS |
| 1237 | TGGAAAC | PBS |
| 1238 | TGGAAACC | PBS |
| 1239 | TGGAAACCC | PBS |
| 1240 | TGGAAACCCA (SEQ ID NO: 1240) | PBS |
| 1241 | TGGAAACCCAG (SEQ ID NO: 1241) | PBS |
| 1242 | TGGAAACCCAGC (SEQ ID NO: 1242) | PBS |
| 1243 | TGGAAACCCAGCA (SEQ ID NO: 1243) | PBS |
| 1244 | TGGAAACCCAGCAA (SEQ ID NO: 1244) | PBS |
| 1245 | TGGAAACCCAGCAAG (SEQ ID NO: 1245) | PBS |
| 1246 | TGGAAACCCAGCAAGA (SEQ ID NO: 1246) | PBS |
| 1247 | TGGAAACCCAGCAAGAC (SEQ ID NO: 1247) | PBS |
| 1248 | TGGAAACCCAGCAAGACA (SEQ ID NO: 1248) | PBS |
| 1249 | TGGAAACCCAGCAAGACAC (SEQ ID NO: 1249) | PBS |
| 1250 | GATCAGGCTAGCACTT (SEQ ID NO: 1250) | RTT*2, 8 (TGG-to-TGC PAM silencing edit) |
| 1251 | GATCAGGCTAGTACTT (SEQ ID NO: 1251) | RTT*1, 3, 8, 9 (TG-to-TA; TGG-to-TAC PAM |
| | | silencing edit) |
| 1252 | GATCAGGCTTCAACTT (SEQ ID NO: 1252) | RTT*1, 4, 8, 10 (TG-to-TT; TGG-to-TTG PAM |
| | | silencing edit) |
| 1253 | GATCAGGCTTCCACTT (SEQ ID NO: 1253) | RTT*1, 5, 8, 11 |
| 1254 | GATCAGGCTTCGACTT (SEQ ID NO: 1254) | RTT*1, 6, 8, 12 (TG-to-TC; TGG-to-TCG PAM |
| | | silencing edit) |
| 1255 | GATCAGGCTTCTACTT (SEQ ID NO: 1255) | RTT*1, 7, 8, 13 (TG-to-TA; TGG-to-TAG PAM |
| | | silencing edit) |
| 1256 | GGATCAGGCTAGCACTT (SEQ ID NO: 1256) | RTT*2, 8 (TGG-to-TGC PAM silencing edit) |
| 1257 | GGATCAGGCTAGTACTT (SEQ ID NO: 1257) | RTT*1, 3, 8, 9 (TG-to-TA; TGG-to-TAC PAM |
| | | silencing edit) |
| 1258 | GGATCAGGCTTCAACTT (SEQ ID NO: 1258) | RTT*1, 4, 8, 10 (TG-to-TT; TGG-to-TTG PAM |
| | | silencing edit) |
| 1259 | GGATCAGGCTTCCACTT (SEQ ID NO: 1259) | RTT*1, 5, 8, 11 |
| 1260 | GGATCAGGCTTCGACTT (SEQ ID NO: 1260) | RTT*1, 6, 8, 12 (TG-to-TC; TGG-to-TCG PAM |
| | | silencing edit) |
| 1261 | GGATCAGGCTTCTACTT (SEQ ID NO: 1261) | RTT*1, 7, 8, 13 (TG-to-TA; TGG-to-TAG PAM |
| | | silencing edit) |
| 1262 | GGGATCAGGCTAGCACTT (SEQ ID NO: 1262) | RTT*2, 8 (TGG-to-TGC PAM silencing edit) |
| 1263 | GGGATCAGGCTAGTACTT (SEQ ID NO: 1263) | RTT*1, 3, 8, 9 (TG-to-TA; TGG-to-TAC PAM |
| | | silencing edit) |
| 1264 | GGGATCAGGCTTCAACTT (SEQ ID NO: 1264) | RTT*1, 4, 8, 10 (TG-to-TT; TGG-to-TTG PAM |
| | | silencing edit) |
| 1265 | GGGATCAGGCTTCCACTT (SEQ ID NO: 1265) | RTT*1, 5, 8, 11 |
| 1266 | GGGATCAGGCTTCGACTT (SEQ ID NO: 1266) | RTT*1, 6, 8, 12 (TG-to-TC; TGG-to-TCG PAM |
| | | silencing edit) |
| 1267 | GGGATCAGGCTTCTACTT (SEQ ID NO: 1267) | RTT*1, 7, 8, 13 (TG-to-TA; TGG-to-TAG PAM |
| | | silencing edit) |
| 1268 | TGGGATCAGGCTAGCACTT (SEQ ID NO: 1268) | RTT*2, 8 (TGG-to-TGC PAM silencing edit) |
| 1269 | TGGGATCAGGCTAGTACTT (SEQ ID NO: 1269) | RTT*1, 3, 8, 9 (TG-to-TA; TGG-to-TAC PAM |
| | | silencing edit) |
| 1270 | TGGGATCAGGCTTCAACTT (SEQ ID NO: 1270) | RTT*1, 4, 8, 10 (TG-to-TT; TGG-to-TTG PAM |
| | | silencing edit) |
| 1271 | TGGGATCAGGCTTCCACTT (SEQ ID NO: 1271) | RTT*1, 5, 8, 11 |
| 1272 | TGGGATCAGGCTTCGACTT (SEQ ID NO: 1272) | RTT*1, 6, 8, 12 (TG-to-TC; TGG-to-TCG PAM |
| | | silencing edit) |
| 1273 | TGGGATCAGGCTTCTACTT (SEQ ID NO: 1273) | RTT*1, 7, 8, 13 (TG-to-TA; TGG-to-TAG PAM |
| | | silencing edit) |
| 1274 | ATGGGATCAGGCTAGCACTT (SEQ ID NO: 1274) | RTT*2, 8 (TGG-to-TGC PAM silencing edit) |
| 1275 | ATGGGATCAGGCTAGTACTT (SEQ ID NO: 1275) | RTT*1, 3, 8, 9 (TG-to-TA; TGG-to-TAC PAM |
| | | silencing edit) |
| 1276 | ATGGGATCAGGCTTCAACTT (SEQ ID NO: 1276) | RTT*1, 4, 8, 10 (TG-to-TT; TGG-to-TTG PAM |
| | | silencing edit) |
| 28 | ATGGGATCAGGCTTCCACTT (SEQ ID NO: 28) | RTT*1, 5, 8, 11 |
| 1277 | ATGGGATCAGGCTTCGACTT (SEQ ID NO: 1277) | RTT*1, 6, 8, 12 (TG-to-TC; TGG-to-TCG PAM |
| | | silencing edit) |
| 1278 | ATGGGATCAGGCTTCTACTT (SEQ ID NO: 1278) | RTT*1, 7, 8, 13 (TG-to-TA; TGG-to-TAG PAM |
| | | silencing edit) |
| 1279 | TATGGGATCAGGCTAGCACTT (SEQ ID NO: 1279) | RTT*2, 8 (TGG-to-TGC PAM silencing edit) |
| 1280 | TATGGGATCAGGCTAGTACTT (SEQ ID NO: 1280) | RTT*1, 3, 8, 9 (TG-to-TA; TGG-to-TAC PAM |
| | | silencing edit) |
| 1281 | TATGGGATCAGGCTTCAACTT (SEQ ID NO: 1281) | RTT*1, 4, 8, 10 (TG-to-TT; TGG-to-TTG PAM |
| | | silencing edit) |
| 1282 | TATGGGATCAGGCTTCCACTT (SEQ ID NO: 1282) | RTT*1, 5, 8, 11 |
| 1283 | TATGGGATCAGGCTTCGACTT (SEQ ID NO: 1283) | RTT*1, 6, 8, 12 (TG-to-TC; TGG-to-TCG PAM |
| | | silencing edit) |
| 1284 | TATGGGATCAGGCTTCTACTT (SEQ ID NO: 1284) | RTT*1, 7, 8, 13 (TG-to-TA; TGG-to-TAG PAM |
| | | silencing edit) |
| 1285 | GTATGGGATCAGGCTAGCACTT (SEQ ID NO: | RTT*2, 8 (TGG-to-TGC PAM silencing edit) |
| | 1285) | |
| 1286 | GTATGGGATCAGGCTAGTACTT (SEQ ID NO: 1286) | RTT*1, 3, 8, 9 (TG-to-TA; TGG-to-TAC PAM |
| | | silencing edit) |
| 1287 | GTATGGGATCAGGCTTCAACTT (SEQ ID NO: 1287) | RTT*1, 4, 8, 10 (TG-to-TT; TGG-to-TTG PAM |
| | | silencing edit) |
| 1288 | GTATGGGATCAGGCTTCCACTT (SEQ ID NO: 1288) | RTT*1, 5, 8, 11 |
| 1289 | GTATGGGATCAGGCTTCGACTT (SEQ ID NO: 1289) | RTT*1, 6, 8, 12 (TG-to-TC; TGG-to-TCG PAM |
| | | silencing edit) |
| 1290 | GTATGGGATCAGGCTTCTACTT (SEQ ID NO: 1290) | RTT*1, 7, 8, 13 (TG-to-TA; TGG-to-TAG PAM |
| | | silencing edit) |
| 1291 | TGTATGGGATCAGGCTAGCACTT (SEQ ID NO: | RTT*2, 8 (TGG-to-TGC PAM silencing edit) |
| | 1291) | |
| 1292 | TGTATGGGATCAGGCTAGTACTT (SEQ ID NO: | RTT*1, 3, 8, 9 (TG-to-TA; TGG-to-TAC PAM |
| | 1292) | silencing edit) |
| 1293 | TGTATGGGATCAGGCTTCAACTT (SEQ ID NO: | RTT*1, 4, 8, 10 (TG-to-TT; TGG-to-TTG PAM |
| | 1293) | silencing edit) |
| 1294 | TGTATGGGATCAGGCTTCCACTT (SEQ ID NO: | RTT*1, 5, 8, 11 |
| | 1294) | |
| 1295 | TGTATGGGATCAGGCTTCGACTT (SEQ ID NO: | RTT*1, 6, 8, 12 (TG-to-TC; TGG-to-TCG PAM |
| | 1295) | silencing edit) |
| 1296 | TGTATGGGATCAGGCTTCTACTT (SEQ ID NO: | RTT*1, 7, 8, 13 (TG-to-TA; TGG-to-TAG PAM |
| | 1296) | silencing edit) |
| 1297 | CTGTATGGGATCAGGCTAGCACTT (SEQ ID NO: | RTT*2, 8 (TGG-to-TGC PAM silencing edit) |
| | 1297) | |
| 1298 | CTGTATGGGATCAGGCTAGTACTT (SEQ ID NO: | RTT*1, 3, 8, 9 (TG-to-TA; TGG-to-TAC PAM |
| | 1298) | silencing edit) |
| 1299 | CTGTATGGGATCAGGCTTCAACTT (SEQ ID NO: | RTT*1, 4, 8, 10 (TG-to-TT; TGG-to-TTG PAM |
| | 1299) | silencing edit) |
| 1300 | CTGTATGGGATCAGGCTTCCACTT (SEQ ID NO: | RTT*1, 5, 8, 11 |
| | 1300) | |
| 1301 | CTGTATGGGATCAGGCTTCGACTT (SEQ ID NO: | RTT*1, 6, 8, 12 (TG-to-TC; TGG-to-TCG PAM |
| | 1301) | silencing edit) |
| 1302 | CTGTATGGGATCAGGCTTCTACTT (SEQ ID NO: | RTT*1, 7, 8, 13 (TG-to-TA; TGG-to-TAG PAM |
| | 1302) | silencing edit) |
| 1303 | TCTGTATGGGATCAGGCTAGCACTT (SEQ ID NO: | RTT*2, 8 (TGG-to-TGC PAM silencing edit) |
| | 1303) | |
| 1304 | TCTGTATGGGATCAGGCTAGTACTT (SEQ ID NO: | RTT*1, 3, 8, 9 (TG-to-TA; TGG-to-TAC PAM |
| | 1304) | silencing edit) |
| 1305 | TCTGTATGGGATCAGGCTTCAACTT (SEQ ID NO: | RTT*1, 4, 8, 10 (TG-to-TT; TGG-to-TTG PAM |
| | 1305) | silencing edit) |
| 1306 | TCTGTATGGGATCAGGCTTCCACTT (SEQ ID NO: | RTT*1, 5, 8, 11 |
| | 1306) | |
| 1307 | TCTGTATGGGATCAGGCTTCGACTT (SEQ ID NO: | RTT*1, 6, 8, 12 (TG-to-TC; TGG-to-TCG PAM |
| | 1307) | silencing edit) |
| 1308 | TCTGTATGGGATCAGGCTTCTACTT (SEQ ID NO: | RTT*1, 7, 8, 13 (TG-to-TA; TGG-to-TAG PAM |
| | 1308) | silencing edit) |
| 1309 | TTCTGTATGGGATCAGGCTAGCACTT (SEQ ID NO: | RTT*2, 8 (TGG-to-TGC PAM silencing edit) |
| | 1309) | |
| 1310 | TTCTGTATGGGATCAGGCTAGTACTT (SEQ ID NO: | RTT*1, 3, 8, 9 (TG-to-TA; TGG-to-TAC PAM |
| | 1310) | silencing edit) |
| 1311 | TTCTGTATGGGATCAGGCTTCAACTT (SEQ ID NO: | RTT*1, 4, 8, 10 (TG-to-TT; TGG-to-TTG PAM |
| | 1311) | silencing edit) |
| 1312 | TTCTGTATGGGATCAGGCTTCCACTT (SEQ ID NO: | RTT*1, 5, 8, 11 |
| | 1312) | |
| 1313 | TTCTGTATGGGATCAGGCTTCGACTT (SEQ ID NO: | RTT*1, 6, 8, 12 (TG-to-TC; TGG-to-TCG PAM |
| | 1313) | silencing edit) |
| 1314 | TTCTGTATGGGATCAGGCTTCTACTT (SEQ ID NO: | RTT*1, 7, 8, 13 (TG-to-TA; TGG-to-TAG PAM |
| | 1314) | silencing edit) |
| 1315 | TTTCTGTATGGGATCAGGCTAGCACTT (SEQ ID | RTT*2, 8 (TGG-to-TGC PAM silencing edit) |
| | NO: 1315) | |
| 1316 | TTTCTGTATGGGATCAGGCTAGTACTT (SEQ ID | RTT*1, 3, 8, 9 (TG-to-TA; TGG-to-TAC PAM |
| | NO: 1316) | silencing edit) |
| 1317 | TTTCTGTATGGGATCAGGCTTCAACTT (SEQ ID | RTT*1, 4, 8, 10 (TG-to-TT; TGG-to-TTG PAM |
| | NO: 1317) | silencing edit) |
| 1318 | TTTCTGTATGGGATCAGGCTTCCACTT (SEQ ID | RTT*1, 5, 8, 11 |
| | NO: 1318) | |
| 1319 | TTTCTGTATGGGATCAGGCTTCGACTT (SEQ ID | RTT*1, 6, 8, 12 (TG-to-TC; TGG-to-TCG PAM |
| | NO: 1319) | silencing edit) |
| 1320 | TTTCTGTATGGGATCAGGCTTCTACTT (SEQ ID | RTT*1, 7, 8, 13 (TG-to-TA; TGG-to-TAG PAM |
| | NO: 1320) | silencing edit) |
| 1321 | CTTTCTGTATGGGATCAGGCTAGCACTT (SEQ ID | RTT*2, 8 (TGG-to-TGC PAM silencing edit) |
| | NO: 1321) | |
| 1322 | CTTTCTGTATGGGATCAGGCTAGTACTT (SEQ ID | RTT*1, 3, 8, 9 (TG-to-TA; TGG-to-TAC PAM |
| | NO: 1322) | silencing edit) |
| 1323 | CTTTCTGTATGGGATCAGGCTTCAACTT (SEQ ID | RTT*1, 4, 8, 10 (TG-to-TT; TGG-to-TTG PAM |
| | NO: 1323) | silencing edit) |
| 1324 | CTTTCTGTATGGGATCAGGCTTCCACTT (SEQ ID | RTT*1, 5, 8, 11 |
| | NO: 1324) | |
| 1325 | CTTTCTGTATGGGATCAGGCTTCGACTT (SEQ ID | RTT*1, 6, 8, 12 (TG-to-TC; TGG-to-TCG PAM |
| | NO: 1325) | silencing edit) |
| 1326 | CTTTCTGTATGGGATCAGGCTTCTACTT (SEQ ID | RTT*1, 7, 8, 13 (TG-to-TA; TGG-to-TAG PAM |
| | NO: 1326) | silencing edit) |
| 1327 | GCTTTCTGTATGGGATCAGGCTAGCACTT (SEQ ID | RTT*2, 8 (TGG-to-TGC PAM silencing edit) |
| | NO: 1327) | |
| 1328 | GCTTTCTGTATGGGATCAGGCTAGTACTT (SEQ ID | RTT*1, 3, 8, 9 (TG-to-TA; TGG-to-TAC PAM |
| | NO: 1328) | silencing edit) |
| 1329 | GCTTTCTGTATGGGATCAGGCTTCAACTT (SEQ ID | RTT*1, 4, 8, 10 (TG-to-TT; TGG-to-TTG PAM |
| | NO: 1329) | silencing edit) |
| 1330 | GCTTTCTGTATGGGATCAGGCTTCCACTT (SEQ ID | RTT*1, 5, 8, 11 |
| | NO: 1330) | |
| 1331 | GCTTTCTGTATGGGATCAGGCTTCGACTT (SEQ ID | RTT*1, 6, 8, 12 (TG-to-TC; TGG-to-TCG PAM |
| | NO: 1331) | silencing edit) |
| 1332 | GCTTTCTGTATGGGATCAGGCTTCTACTT (SEQ ID | RTT*1, 7, 8, 13 (TG-to-TA; TGG-to-TAG PAM |
| | NO: 1332) | silencing edit) |
| 1333 | AGCTTTCTGTATGGGATCAGGCTAGCACTT (SEQ | RTT*2, 8 (TGG-to-TGC PAM silencing edit) |
| | ID NO: 1333) | |
| 1334 | AGCTTTCTGTATGGGATCAGGCTAGTACTT (SEQ | RTT*1, 3, 8, 9 (TG-to-TA; TGG-to-TAC PAM |
| | ID NO: 1334) | silencing edit) |
| 1335 | AGCTTTCTGTATGGGATCAGGCTTCAACTT (SEQ | RTT*1, 4, 8, 10 (TG-to-TT; TGG-to-TTG PAM |
| | ID NO: 1335) | silencing edit) |
| 1336 | AGCTTTCTGTATGGGATCAGGCTTCCACTT (SEQ | RTT*1, 5, 8, 11 |
| | ID NO: 1336) | |
| 1337 | AGCTTTCTGTATGGGATCAGGCTTCGACTT (SEQ | RTT*1, 6, 8, 12 (TG-to-TC; TGG-to-TCG PAM |
| | ID NO: 1337) | silencing edit) |
| 1338 | AGCTTTCTGTATGGGATCAGGCTTCTACTT (SEQ | RTT*1, 7, 8, 13 (TG-to-TA; TGG-to-TAG PAM |
| | ID NO: 1338) | silencing edit) |
| 1339 | AAGCTTTCTGTATGGGATCAGGCTAGCACTT | RTT*2, 8 (TGG-to-TGC PAM silencing edit) |
| | (SEQ ID NO: 1339) | |
| 1340 | AAGCTTTCTGTATGGGATCAGGCTAGTACTT | RTT*1, 3, 8, 9 (TG-to-TA; TGG-to-TAC PAM |
| | (SEQ ID NO: 1340) | silencing edit) |
| 1341 | AAGCTTTCTGTATGGGATCAGGCTTCAACTT (SEQ | RTT*1, 4, 8, 10 (TG-to-TT; TGG-to-TTG PAM |
| | ID NO: 1341) | silencing edit) |
| 1342 | AAGCTTTCTGTATGGGATCAGGCTTCCACTT (SEQ | RTT*1, 5, 8, 11 |
| | ID NO: 1342) | |
| 1343 | AAGCTTTCTGTATGGGATCAGGCTTCGACTT (SEQ | RTT*1, 6, 8, 12 (TG-to-TC; TGG-to-TCG PAM |
| | ID NO: 1343) | silencing edit) |
| 1344 | AAGCTTTCTGTATGGGATCAGGCTTCTACTT (SEQ | RTT*1, 7, 8, 13 (TG-to-TA; TGG-to-TAG PAM |
| | ID NO: 1344) | silencing edit) |
| 1345 | GAAGCTTTCTGTATGGGATCAGGCTAGCACTT | RTT*2, 8 (TGG-to-TGC PAM silencing edit) |
| | (SEQ ID NO: 1345) | |
| 1346 | GAAGCTTTCTGTATGGGATCAGGCTAGTACTT | RTT*1, 3, 8, 9 (TG-to-TA; TGG-to-TAC PAM |
| | (SEQ ID NO: 1346) | silencing edit) |
| 1347 | GAAGCTTTCTGTATGGGATCAGGCTTCAACTT | RTT*1, 4, 8, 10 (TG-to-TT; TGG-to-TTG PAM |
| | (SEQ ID NO: 1347) | silencing edit) |
| 1348 | GAAGCTTTCTGTATGGGATCAGGCTTCCACTT | RTT*1, 5, 8, 11 |
| | (SEQ ID NO: 1348) | |
| 1349 | GAAGCTTTCTGTATGGGATCAGGCTTCGACTT | RTT*1, 6, 8, 12 (TG-to-TC; TGG-to-TCG PAM |
| | (SEQ ID NO: 1349) | silencing edit) |
| 1350 | GAAGCTTTCTGTATGGGATCAGGCTTCTACTT | RTT*1, 7, 8, 13 (TG-to-TA; TGG-to-TAG PAM |
| | (SEQ ID NO: 1350) | silencing edit) |
| 1351 | AGAAGCTTTCTGTATGGGATCAGGCTAGCACTT | RTT*2, 8 (TGG-to-TGC PAM silencing edit) |
| | (SEQ ID NO: 1351) | |
| 1352 | AGAAGCTTTCTGTATGGGATCAGGCTAGTACTT | RTT*1, 3, 8, 9 (TG-to-TA; TGG-to-TAC PAM |
| | (SEQ ID NO: 1352) | silencing edit) |
| 1353 | AGAAGCTTTCTGTATGGGATCAGGCTTCAACTT | RTT*1, 4, 8, 10 (TG-to-TT; TGG-to-TTG PAM |
| | (SEQ ID NO: 1353) | silencing edit) |
| 1354 | AGAAGCTTTCTGTATGGGATCAGGCTTCCACTT | RTT*1, 5, 8, 11 |
| | (SEQ ID NO: 1354) | |
| 1355 | AGAAGCTTTCTGTATGGGATCAGGCTTCGACTT | RTT*1, 6, 8, 12 (TG-to-TC; TGG-to-TCG PAM |
| | SEQ ID NO: 1355) | silencing edit) |
| 1356 | AGAAGCTTTCTGTATGGGATCAGGCTTCTACTT | RTT*1, 7, 8, 13 (TG-to-TA; TGG-to-TAG PAM |
| | (SEQ ID NO: 1356) | silencing edit) |
| 1357 | CAGAAGCTTTCTGTATGGGATCAGGCTAGCACTT | RTT*2, 8 (TGG-to-TGC PAM silencing edit) |
| | (SEQ ID NO: 1357) | |
| 1358 | CAGAAGCTTTCTGTATGGGATCAGGCTAGTACTT | RTT*1, 3, 8, 9 (TG-to-TA; TGG-to-TAC PAM |
| | (SEQ ID NO: 1358) | silencing edit) |
| 1359 | CAGAAGCTTTCTGTATGGGATCAGGCTTCAACTT | RTT*1, 4, 8, 10 (TG-to-TT; TGG-to-TTG PAM |
| | (SEQ ID NO: 1359) | silencing edit) |
| 1360 | CAGAAGCTTTCTGTATGGGATCAGGCTTCCACTT | RTT*1, 5, 8, 11 |
| | (SEQ ID NO: 1360) | |
| 1361 | CAGAAGCTTTCTGTATGGGATCAGGCTTCGACTT | RTT*1, 6, 8, 12 (TG-to-TC; TGG-to-TCG PAM |
| | (SEQ ID NO: 1361) | silencing edit) |
| 1362 | CAGAAGCTTTCTGTATGGGATCAGGCTTCTACTT | RTT*1, 7, 8, 13 (TG-to-TA; TGG-to-TAG PAM |
| | (SEQ ID NO: 1362) | silencing edit) |
| 1363 | GCAGAAGCTTTCTGTATGGGATCAGGCTAGCACT | RTT*2, 8 (TGG-to-TGC PAM silencing edit) |
| | T (SEQ ID NO: 1363) | |
| 1364 | GCAGAAGCTTTCTGTATGGGATCAGGCTAGTACT | RTT*1, 3, 8, 9 (TG-to-TA; TGG-to-TAC PAM |
| | T (SEQ ID NO: 1364) | silencing edit) |
| 1365 | GCAGAAGCTTTCTGTATGGGATCAGGCTTCAACT | RTT*1, 4, 8, 10 (TG-to-TT; TGG-to-TTG PAM |
| | T (SEQ ID NO: 1365) | silencing edit) |
| 1366 | GCAGAAGCTTTCTGTATGGGATCAGGCTTCCACT | RTT*1, 5, 8, 11 |
| | T (SEQ ID NO: 1366) | |
| 1367 | GCAGAAGCTTTCTGTATGGGATCAGGCTTCGACT | RTT*1, 6, 8, 12 (TG-to-TC; TGG-to-TCG PAM |
| | T (SEQ ID NO: 1367) | silencing edit) |
| 1368 | GCAGAAGCTTTCTGTATGGGATCAGGCTTCTACT | RTT*1, 7, 8, 13 (TG-to-TA; TGG-to-TAG PAM |
| | T (SEQ ID NO: 1368) | silencing edit) |
| 1369 | TGCAGAAGCTTTCTGTATGGGATCAGGCTAGCAC | RTT*2, 8 (TGG-to-TGC PAM silencing edit) |
| | TT (SEQ ID NO: 1369) | |
| 1370 | TGCAGAAGCTTTCTGTATGGGATCAGGCTAGTAC | RTT*1, 3, 8, 9 (TG-to-TA; TGG-to-TAC PAM |
| | TT (SEQ ID NO: 1370) | silencing edit) |
| 1371 | TGCAGAAGCTTTCTGTATGGGATCAGGCTTCAAC | RTT*1, 4, 8, 10 (TG-to-TT; TGG-to-TTG PAM |
| | TT (SEQ ID NO: 1371) | silencing edit) |
| 1372 | TGCAGAAGCTTTCTGTATGGGATCAGGCTTCCAC | RTT*1, 5, 8, 11 |
| | TT (SEQ ID NO: 1372) | |
| 1373 | TGCAGAAGCTTTCTGTATGGGATCAGGCTTCGAC | RTT*1, 6, 8, 12 (TG-to-TC; TGG-to-TCG PAM |
| | TT (SEQ ID NO: 1373) | silencing edit) |
| 1374 | TGCAGAAGCTTTCTGTATGGGATCAGGCTTCTAC | RTT*1, 7, 8, 13 (TG-to-TA; TGG-to-TAG PAM |
| | TT (SEQ ID NO: 1374) | silencing edit) |
| 1375 | ATGCAGAAGCTTTCTGTATGGGATCAGGCTAGCA | RTT*2, 8 (TGG-to-TGC PAM silencing edit) |
| | CTT (SEQ ID NO: 1375) | |
| 1376 | ATGCAGAAGCTTTCTGTATGGGATCAGGCTAGTA | RTT*1, 3, 8, 9 (TG-to-TA; TGG-to-TAC PAM |
| | CTT (SEQ ID NO: 1376) | silencing edit) |
| 1377 | ATGCAGAAGCTTTCTGTATGGGATCAGGCTTCAA | RTT*1, 4, 8, 10 (TG-to-TT; TGG-to-TTG PAM |
| | CTT (SEQ ID NO: 1377) | silencing edit) |
| 1378 | ATGCAGAAGCTTTCTGTATGGGATCAGGCTTCCA | RTT*1, 5, 8, 11 |
| | CTT (SEQ ID NO: 1378) | |
| 1379 | ATGCAGAAGCTTTCTGTATGGGATCAGGCTTCGA | RTT*1, 6, 8, 12 (TG-to-TC; TGG-to-TCG PAM |
| | CTT (SEQ ID NO: 1379) | silencing edit) |
| 1380 | ATGCAGAAGCTTTCTGTATGGGATCAGGCTTCTA | RTT*1, 7, 8, 13 (TG-to-TA; TGG-to-TAG PAM |
| | CTT (SEQ ID NO: 1380) | silencing edit) |
| 1381 | GATGCAGAAGCTTTCTGTATGGGATCAGGCTAGC | RTT*2, 8 (TGG-to-TGC PAM silencing edit) |
| | ACTT (SEQ ID NO: 1381) | |
| 1382 | GATGCAGAAGCTTTCTGTATGGGATCAGGCTAGT | RTT*1, 3, 8, 9 (TG-to-TA; TGG-to-TAC PAM |
| | ACTT (SEQ ID NO: 1382) | silencing edit) |
| 1383 | GATGCAGAAGCTTTCTGTATGGGATCAGGCTTCA | RTT*1, 4, 8, 10 (TG-to-TT; TGG-to-TTG PAM |
| | ACTT (SEQ ID NO: 1383) | silencing edit) |
| 1384 | GATGCAGAAGCTTTCTGTATGGGATCAGGCTTCC | RTT*1, 5, 8, 11 |
| | ACTT (SEQ ID NO: 1384) | |
| 1385 | GATGCAGAAGCTTTCTGTATGGGATCAGGCTTCG | RTT*1, 6, 8, 12 (TG-to-TC; TGG-to-TCG PAM |
| | ACTT (SEQ ID NO: 1385) | silencing edit) |
| 1386 | GATGCAGAAGCTTTCTGTATGGGATCAGGCTTCT | RTT*1, 7, 8, 13 (TG-to-TA; TGG-to-TAG PAM |
| | ACTT (SEQ ID NO: 1386) | silencing edit) |
| 1387 | GGATGCAGAAGCTTTCTGTATGGGATCAGGCTAG | RTT*2, 8 (TGG-to-TGC PAM silencing edit) |
| | CACTT (SEQ ID NO: 1387) | |
| 1388 | GGATGCAGAAGCTTTCTGTATGGGATCAGGCTAG | RTT*1, 3, 8, 9 (TG-to-TA; TGG-to-TAC PAM |
| | TACTT (SEQ ID NO: 1388) | silencing edit) |
| 1389 | GGATGCAGAAGCTTTCTGTATGGGATCAGGCTTC | RTT*1, 4, 8, 10 (TG-to-TT; TGG-to-TTG PAM |
| | AACTT (SEQ ID NO: 1389) | silencing edit) |
| 1390 | GGATGCAGAAGCTTTCTGTATGGGATCAGGCTTC | RTT*1, 5, 8, 11 |
| | CACTT (SEQ ID NO: 1390) | |
| 1391 | GGATGCAGAAGCTTTCTGTATGGGATCAGGCTTC | RTT*1, 6, 8, 12 (TG-to-TC; TGG-to-TCG PAM |
| | GACTT (SEQ ID NO: 1391) | silencing edit) |
| 1392 | GGATGCAGAAGCTTTCTGTATGGGATCAGGCTTC | RTT*1, 7, 8, 13 (TG-to-TA; TGG-to-TAG PAM |
| | TACTT (SEQ ID NO: 1392) | silencing edit) |
| 1393 | TGGATGCAGAAGCTTTCTGTATGGGATCAGGCTA | RTT*2, 8 (TGG-to-TGC PAM silencing edit) |
| | GCACTT (SEQ ID NO: 1393) | |
| 1394 | TGGATGCAGAAGCTTTCTGTATGGGATCAGGCTA | RTT*1, 3, 8, 9 (TG-to-TA; TGG-to-TAC PAM |
| | GTACTT (SEQ ID NO: 1394) | silencing edit) |
| 1395 | TGGATGCAGAAGCTTTCTGTATGGGATCAGGCTT | RTT*1, 4, 8, 10 (TG-to-TT; TGG-to-TTG PAM |
| | CAACTT (SEQ ID NO: 1395) | silencing edit) |
| 1396 | TGGATGCAGAAGCTTTCTGTATGGGATCAGGCTT | RTT*1, 5, 8, 11 |
| | CCACTT (SEQ ID NO: 1396) | |
| 1397 | TGGATGCAGAAGCTTTCTGTATGGGATCAGGCTT | RTT*1, 6, 8, 12 (TG-to-TC; TGG-to-TCG PAM |
| | CGACTT (SEQ ID NO: 1397) | silencing edit) |
| 1398 | TGGATGCAGAAGCTTTCTGTATGGGATCAGGCTT | RTT*1, 7, 8, 13 (TG-to-TA; TGG-to-TAG PAM |
| | CTACTT (SEQ ID NO: 1398) | silencing edit) |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b*1 ngRNA spacer (GG PAM) |
| 27 | AGATCTTTCTTGTGATTATC (SEQ ID NO: 27) | PE3 ngRNA spacer (AG PAM) |
| 1274 | ATGGGATCAGGCTAGCACTT (SEQ ID NO: 1274) | PE3b*2 ngRNA spacer (TGG PAM) |
| 1275 | ATGGGATCAGGCTAGTACTT (SEQ ID NO: 1275) | PE3b*3 ngRNA spacer (TG; TGG PAM) |
| 1276 | ATGGGATCAGGCTTCAACTT (SEQ ID NO: 1276) | PE3b*4 ngRNA spacer (TG; TGG PAM) |
| 28 | ATGGGATCAGGCTTCCACTT (SEQ ID NO: 28) | PE3b*5 ngRNA spacer (TG; TGG PAM) |
| 1277 | ATGGGATCAGGCTTCGACTT (SEQ ID NO: 1277) | PE3b*6 ngRNA spacer (TG; TGG PAM) |
| 1278 | ATGGGATCAGGCTTCTACTT (SEQ ID NO: 1278) | PE3b*7 ngRNA spacer (TG; TGG PAM) |
| 1108 | CAGTGCTGGACAGGCTGCTG (SEQ ID NO: 1108) | PE3 ngRNA spacer (TG PAM) |
| 29 | CCTTTTCGCTTTTTCCAAGA (SEQ ID NO: 29) | PE3 ngRNA spacer (TG; TGG PAM) |
| 1110 | CTGGACAGGCTGCTGTGAAG (SEQ ID NO: 1110) | PE3 ngRNA spacer (GG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b*8 ngRNA spacer (AG; AGG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 1111 | GCTGGACAGGCTGCTGTGAA (SEQ ID NO: 1111) | PE3 ngRNA spacer (GG; GGG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG; TGG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 41 | TGATTATCAGTTTTGGATGC (SEQ ID NO: 41) | PE3 ngRNA spacer (AG PAM) |
| 1113 | TGCTGGACAGGCTGCTGTGA (SEQ ID NO: 1113) | PE3 ngRNA spacer (AG; AGG PAM) |
| 1399 | TGGGATCAGGCTAGTACTTT (SEQ ID NO: 1399) | PE3b*9 ngRNA spacer (GG PAM) |
| 1400 | TGGGATCAGGCTTCAACTTT (SEQ ID NO: 1400) | PE3b*10 ngRNA spacer (GG PAM) |
| 43 | TGGGATCAGGCTTCCACTTT (SEQ ID NO: 43) | PE3b*11 ngRNA spacer (GG PAM) |
| 1401 | TGGGATCAGGCTTCGACTTT (SEQ ID NO: 1401) | PE3b*12 ngRNA spacer (GG PAM) |
| 1402 | TGGGATCAGGCTTCTACTTT (SEQ ID NO: 1402) | PE3b*13 ngRNA spacer (GG PAM) |
| 44 | TTATCAGTTTTGGATGCAGA (SEQ ID NO: 44) | PE3 ngRNA spacer (AG PAM) |
| 1114 | TTCAGTGCTGGACAGGCTGC (SEQ ID NO: 1114) | PE3 ngRNA spacer (TG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG; TGG PAM) |
| 48 | TTTTGGATGCAGAAGCTTTC (SEQ ID NO: 48) | PE3 ngRNA spacer (TG PAM) |
| 1403 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACC (SEQ ID NO: | |
| | 1403) | |
| 1404 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCA (SEQ ID NO: | |
| | 1404) | |
| 1405 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACC (SEQ ID NO: | |
| | 1405) | |
| 1406 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGC (SEQ ID | |
| | NO: 1406) | |
| 1407 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCA (SEQ ID | |
| | NO: 1407) | |
| 1408 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCTTTT (SEQ ID | |
| | NO: 1408) | |
| 1409 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACC (SEQ ID | |
| | NO: 1409) | |
| 1410 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAA (SEQ | |
| | ID NO: 1410) | |
| 1411 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGC (SEQ | |
| | ID NO: 1411) | |
| 1412 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCATTTT (SEQ ID | |
| | NO: 1412) | |
| 1413 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCTTTT (SEQ ID | |
| | NO: 1413) | |
| 1414 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCA (SEQ | |
| | ID NO: 1414) | |
| 1415 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACC (SEQ | |
| | ID NO: 1415) | |
| 1416 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAA | |
| | (SEQ ID NO: 1416) | |
| 1417 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCTTTT (SEQ | |
| | ID NO: 1417) | |
| 1418 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCATTTT (SEQ | |
| | ID NO: 1418) | |
| 1419 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGC | |
| | (SEQ ID NO: 1419) | |
| 1420 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCA | |
| | (SEQ ID NO: 1420) | |
| 1421 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACC | |
| | (SEQ ID NO: 1421) | |
| 1422 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCTTTT (SEQ | |
| | ID NO: 1422) | |
| 1423 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAATTTT | |
| | (SEQ ID NO: 1423) | |
| 1424 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCTTTT | |
| | (SEQ ID NO: 1424) | |
| 1425 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | (SEQ ID NO: 1425) | |
| 1426 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGC | |
| | (SEQ ID NO: 1426) | |
| 1427 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCCA | |
| | (SEQ ID NO: 1427) | |
| 1428 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCATTTT | |
| | (SEQ ID NO: 1428) | |
| 1429 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCTTTT | |
| | (SEQ ID NO: 1429) | |
| 1430 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAATTT | |
| | T (SEQ ID NO: 1430) | |
| 1431 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | A (SEQ ID NO: 1431) | |
| 1432 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCCAG | |
| | C (SEQ ID NO: 1432) | |
| 1433 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCTTT | |
| | T (SEQ ID NO: 1433) | |
| 1434 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCATTT | |
| | T (SEQ ID NO: 1434) | |
| 1435 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCTTT | |
| | T (SEQ ID NO: 1435) | |
| 1436 | GTCTTGCTGGGTTTCCAAAGGTTTTAGAGCTAGA | pegRNA*1, 5, 8, 11 |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCCAG | |
| | CAA (SEQ ID NO: 1436) | |
| 1437 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAAT | |
| | TTT (SEQ ID NO: 1437) | |
| 1438 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCT | |
| | TTT (SEQ ID NO: 1438) | |
| 1439 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCCAT | |
| | TTT (SEQ ID NO: 1439) | |
| 1440 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | ATTTT (SEQ ID NO: 1440) | |
| 1441 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCCAG | |
| | CTTTT (SEQ ID NO: 1441) | |
| 1442 | GTCTTGCTGGGTTTCCAAAGGTTTTAGAGCTAGA | pegRNA*1, 5, 8, 11, contains 3′ TTTT |
| | AATAGCAAGTTAAAATAAGGCTAGTCCGTTATCA | |
| | ACTTGAAAAAGTGGCACCGAGTCGGTGCGCTTTC | |
| | TGTATGGGATCAGGCTTCCACTTTGGAAACCCAG | |
| | CAATTTT (SEQ ID NO: 1442) | |
| 1443 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591; 3′ TTTT |
| | ATCAGGCTTCCACTTTGGAAACCAACATTGACGC | |
| | GTCTCTACGTGGGGGCGCGTTTT (SEQ ID NO: | |
| | 1443) | |
| 1444 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591; 3′ TTTT |
| | ATCAGGCTTCCACTTTGGAAACCCAAACATTGAC | |
| | GCGTCTCTACGTGGGGGCGCGTTTT (SEQ ID NO: | |
| | 1444) | |
| 1445 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591; 3′ TTTT |
| | GGATCAGGCTTCCACTTTGGAAACCAACATTGAC | |
| | GCGTCTCTACGTGGGGGCGCGTTTT (SEQ ID NO: | |
| | 1445) | |
| 1446 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591; 3′ TTTT |
| | ATCAGGCTTCCACTTTGGAAACCCAGCAACATTG | |
| | ACGCGTCTCTACGTGGGGGCGCGTTTT (SEQ ID | |
| | NO: 1446) | |
| 1447 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591; 3′ TTTT |
| | GGATCAGGCTTCCACTTTGGAAACCCAAACATTG | |
| | ACGCGTCTCTACGTGGGGGCGCGTTTT (SEQ ID | |
| | NO: 1447) | |
| 1448 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591; 3′ TTTT |
| | TGGGATCAGGCTTCCACTTTGGAAACCAACATTG | |
| | ACGCGTCTCTACGTGGGGGCGCGTTTT (SEQ ID | |
| | NO: 1448) | |
| 1449 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591; 3′ TTTT |
| | ATCAGGCTTCCACTTTGGAAACCCAGCAAAACAT | |
| | TGACGCGTCTCTACGTGGGGGCGCGTTTT (SEQ | |
| | ID NO: 1449) | |
| 1450 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591; 3′ TTTT |
| | GGATCAGGCTTCCACTTTGGAAACCCAGCAACAT | |
| | TGACGCGTCTCTACGTGGGGGCGCGTTTT (SEQ | |
| | ID NO: 1450) | |
| 1451 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591; 3′ TTTT |
| | TGGGATCAGGCTTCCACTTTGGAAACCCAAACAT | |
| | TGACGCGTCTCTACGTGGGGGCGCGTTTT (SEQ | |
| | ID NO: 1451) | |
| 1452 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591; 3′ TTTT |
| | TATGGGATCAGGCTTCCACTTTGGAAACCAACAT | |
| | TGACGCGTCTCTACGTGGGGGCGCGTTTT (SEQ | |
| | ID NO: 1452) | |
| 1453 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591; 3′ TTTT |
| | GGATCAGGCTTCCACTTTGGAAACCCAGCAAAA | |
| | CATTGACGCGTCTCTACGTGGGGGCGCGTTTT | |
| | (SEQ ID NO: 1453) | |
| 1454 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591; 3′ TTTT |
| | TGGGATCAGGCTTCCACTTTGGAAACCCAGCAAC | |
| | ATTGACGCGTCTCTACGTGGGGGCGCGTTTT | |
| | (SEQ ID NO: 1454) | |
| 1455 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591; 3′ TTTT |
| | TATGGGATCAGGCTTCCACTTTGGAAACCCAAAC | |
| | ATTGACGCGTCTCTACGTGGGGGCGCGTTTT | |
| | (SEQ ID NO: 1455) | |
| 1456 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591; 3′ TTTT |
| | TGTATGGGATCAGGCTTCCACTTTGGAAACCAAC | |
| | ATTGACGCGTCTCTACGTGGGGGCGCGTTTT | |
| | (SEQ ID NO: 1456) | |
| 1457 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591; 3′ TTTT |
| | TGGGATCAGGCTTCCACTTTGGAAACCCAGCAAA | |
| | ACATTGACGCGTCTCTACGTGGGGGCGCGTTTT | |
| | (SEQ ID NO: 1457) | |
| 1458 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591; 3′ TTTT |
| | TATGGGATCAGGCTTCCACTTTGGAAACCCAGCA | |
| | ACATTGACGCGTCTCTACGTGGGGGCGCGTTTT | |
| | (SEQ ID NO: 1458) | |
| 1459 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591; 3′ TTTT |
| | TGTATGGGATCAGGCTTCCACTTTGGAAACCCAA | |
| | ACATTGACGCGTCTCTACGTGGGGGCGCGTTTT | |
| | (SEQ ID NO: 1459) | |
| 1460 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591; 3′ TTTT |
| | TATGGGATCAGGCTTCCACTTTGGAAACCCAGCA | |
| | AAACATTGACGCGTCTCTACGTGGGGGCGCGTTT | |
| | T (SEQ ID NO: 1460) | |
| 1461 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591; 3′ TTTT |
| | TGTATGGGATCAGGCTTCCACTTTGGAAACCCAG | |
| | CAACATTGACGCGTCTCTACGTGGGGGCGCGTTT | |
| | T (SEQ ID NO: 1461) | |
| 1462 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591; 3′ TTTT |
| | TGTATGGGATCAGGCTTCCACTTTGGAAACCCAG | |
| | CAAAACATTGACGCGTCTCTACGTGGGGGCGCGT | |
| | TTT (SEQ ID NO: 1462) | |
| 49 | TTTCTTGTGATTATCAGTTTGTTTTAGAGCTAGAA | PE3 ngRNA (TG; TGG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b*8 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b*5 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG; TGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b*8 ngRNA (AG; AGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG; TGG PAM), contains 3′ |
| | AATAGCAAGTTAAAATAAGGCTAGTCCGTTATCA | TTTT; 5′G |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b*5 ngRNA (TG; TGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b*8 ngRNA (AG; AGG PAM), contains |
| | AATAGCAAGTTAAAATAAGGCTAGTCCGTTATCA | linker sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT; 5′G |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b*5 ngRNA (TG; TGG PAM), contains |
| | AAATAGCAAGTTAAAATAAGGCTAGTCCGTTATC | linker sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 60) | |
| 3631 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591 |
| | ATCAGGCTTCCACTTTGGAAACCAACATTGACGC | |
| | GTCTCTACGTGGGGGCGCG (SEQ ID NO: 3638) | |
| 3632 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591 |
| | ATCAGGCTTCCACTTTGGAAACCCAAACATTGAC | |
| | GCGTCTCTACGTGGGGGCGCG (SEQ ID NO: 3639) | |
| 3633 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591 |
| | GGATCAGGCTTCCACTTTGGAAACCAACATTGAC | |
| | GCGTCTCTACGTGGGGGCGCG (SEQ ID NO: 3640) | |
| 3634 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591 |
| | ATCAGGCTTCCACTTTGGAAACCCAGCAACATTG | |
| | ACGCGTCTCTACGTGGGGGCGCG (SEQ ID | |
| | NO: 3641) | |
| 3635 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591 |
| | GGATCAGGCTTCCACTTTGGAAACCCAAACATTG | |
| | ACGCGTCTCTACGTGGGGGCGCG (SEQ ID | |
| | NO: 3642) | |
| 3636 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591 |
| | TGGGATCAGGCTTCCACTTTGGAAACCAACATTG | |
| | ACGCGTCTCTACGTGGGGGCGCG (SEQ ID | |
| | NO: 3643) | |
| 3637 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCATGGG | NO: 3591 |
| | ATCAGGCTTCCACTTTGGAAACCCAGCAAAACAT | |
| | TGACGCGTCTCTACGTGGGGGCGCG (SEQ ID | |
| | NO: 3644) | |
| 3638 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591 |
| | GGATCAGGCTTCCACTTTGGAAACCCAGCAACAT | |
| | TGACGCGTCTCTACGTGGGGGCGCG (SEQ ID | |
| | NO: 3645) | |
| 3639 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591 |
| | TGGGATCAGGCTTCCACTTTGGAAACCCAAACAT | |
| | TGACGCGTCTCTACGTGGGGGCGCG (SEQ ID | |
| | NO: 3646) | |
| 3640 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591 |
| | TATGGGATCAGGCTTCCACTTTGGAAACCAACAT | |
| | TGACGCGTCTCTACGTGGGGGCGCG (SEQ ID | |
| | NO: 3647) | |
| 3641 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGTATG | NO: 3591 |
| | GGATCAGGCTTCCACTTTGGAAACCCAGCAAAA | |
| | CATTGACGCGTCTCTACGTGGGGGCGCG (SEQ ID | |
| | NO: 3648) | |
| 3642 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591 |
| | TGGGATCAGGCTTCCACTTTGGAAACCCAGCAAC | |
| | ATTGACGCGTCTCTACGTGGGGGCGCG (SEQ ID | |
| | NO: 3649) | |
| 3643 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591 |
| | TATGGGATCAGGCTTCCACTTTGGAAACCCAAAC | |
| | ATTGACGCGTCTCTACGTGGGGGCGCG (SEQ ID | |
| | NO: 3650) | |
| 3644 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591 |
| | TGTATGGGATCAGGCTTCCACTTTGGAAACCAAC | |
| | ATTGACGCGTCTCTACGTGGGGGCGCG (SEQ ID | |
| | NO: 3651) | |
| 3645 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTCTGTA | NO: 3591 |
| | TGGGATCAGGCTTCCACTTTGGAAACCCAGCAAA | |
| | ACATTGACGCGTCTCTACGTGGGGGCGCG (SEQ | |
| | ID NO: 3652) | |
| 3646 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591 |
| | TATGGGATCAGGCTTCCACTTTGGAAACCCAGCA | |
| | ACATTGACGCGTCTCTACGTGGGGGCGCG (SEQ | |
| | ID NO: 3653) | |
| 3647 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591 |
| | TGTATGGGATCAGGCTTCCACTTTGGAAACCCAA | |
| | ACATTGACGCGTCTCTACGTGGGGGCGCG (SEQ | |
| | ID NO: 3654) | |
| 3648 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCTTTCTG | NO: 3591 |
| | TATGGGATCAGGCTTCCACTTTGGAAACCCAGCA | |
| | AAACATTGACGCGTCTCTACGTGGGGGCGCG | |
| | (SEQ ID NO: 3655) | |
| 3649 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591 |
| | TGTATGGGATCAGGCTTCCACTTTGGAAACCCAG | |
| | CAACATTGACGCGTCTCTACGTGGGGGCGCG | |
| | (SEQ ID NO: 3656) | |
| 3650 | GTCTTGCTGGGTTTCCAAAGGTTTTAGAGCTAGA | pegRNA*1, 5, 8, 11, contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCGCTTTC | NO: 3591 |
| | TGTATGGGATCAGGCTTCCACTTTGGAAACCCAG | |
| | CAAAACATTGACGCGTCTCTACGTGGGGGCGCG | |
| | (SEQ ID NO: 3657) | |

TABLE 19

| Sequence | | |
|---|---|---|
| Number | Sequence | Description |
| 1463 | AAGACAGGTGTCTTGCT (SEQ ID NO: 1463) | PEgRNA spacer (GG; GGG PAM) |
| 1464 | GAAGACAGGTGTCTTGCT (SEQ ID NO: 1464) | PEgRNA spacer (GG; GGG PAM) |
| 1465 | TGAAGACAGGTGTCTTGCT (SEQ ID NO: 1465) | PEgRNA spacer (GG; GGG PAM) |
| 213 | GTGAAGACAGGTGTCTTGCT (SEQ ID NO: 213) | PEgRNA spacer (GG; GGG PAM) |
| 1466 | CGTGAAGACAGGTGTCTTGCT | PEgRNA spacer (GG; GGG PAM) |
| | (SEQ ID NO: 1466) | |
| 1467 | ACGTGAAGACAGGTGTCTTGCT | PEgRNA spacer (GG; GGG PAM) |
| | (SEQ ID NO: 1467) | |
| 1468 | AAGAC | PBS |
| 1469 | AAGACA | PBS |
| 1470 | AAGACAC | PBS |
| 1471 | AAGACACC | PBS |
| 1472 | AAGACACCT | PBS |
| 1473 | AAGACACCTG (SEQ ID NO: 1473) | PBS |
| 1474 | AAGACACCTGT (SEQ ID NO: 1474) | PBS |
| 1475 | AAGACACCTGTC (SEQ ID NO: 1475) | PBS |
| 1476 | AAGACACCTGTCT (SEQ ID NO: 1476) | PBS |
| 1477 | AAGACACCTGTCTT (SEQ ID NO: 1477) | PBS |
| 1478 | AAGACACCTGTCTTC (SEQ ID NO: 1478) | PBS |
| 1479 | AAGACACCTGTCTTCA (SEQ ID NO: 1479) | PBS |
| 1480 | AAGACACCTGTCTTCAC (SEQ ID NO: 1480) | PBS |
| 1481 | AAGACACCTGTCTTCACG (SEQ ID NO: 1481) | PBS |
| 1482 | AAGACACCTGTCTTCACGT (SEQ ID NO: 1482) | PBS |
| 1483 | GATCAGGCTTCCACTTTGGAAACACAGC (SEQ ID | RTT*1, 2, 3, 4, 7, 10 (GG-to-GT; GGG-to- |
| | NO: 1483) | GTG PAM silencing edit) |
| 1484 | GATCAGGCTTCCACTTTGGAAACCCAGC (SEQ ID | RTT*1, 2, 3, 10 |
| | NO: 1484) | |
| 1485 | GATCAGGCTTCCACTTTGGAAACGCAGC (SEQ ID | RTT*1, 2, 3, 5, 8, 10 (GG-to-GC; GGG-to- |
| | NO: 1485) | GCG PAM silencing edit) |
| 1486 | GATCAGGCTTCCACTTTGGAAACTCAGC (SEQ ID | RTT*1, 2, 3, 6, 9, 10 (GG-to-GA; GGG-to- |
| | NO: 1486) | GAG PAM silencing edit) |
| 1487 | GGATCAGGCTTCCACTTTGGAAACACAGC (SEQ | RTT*1, 2, 3, 4, 7, 10 (GG-to-GT; GGG-to- |
| | ID NO: 1487) | GTG PAM silencing edit) |
| 1488 | GGATCAGGCTTCCACTTTGGAAACCCAGC (SEQ | RTT*1, 2, 3, 10 |
| | ID NO: 1488) | |
| 1489 | GGATCAGGCTTCCACTTTGGAAACGCAGC (SEQ | RTT*1, 2, 3, 5, 8, 10 (GG-to-GC; GGG-to- |
| | ID NO: 1489) | GCG PAM silencing edit) |
| 1490 | GGATCAGGCTTCCACTTTGGAAACTCAGC (SEQ | RTT*1, 2, 3, 6, 9, 10 (GG-to-GA; GGG-to- |
| | ID NO: 1490) | GAG PAM silencing edit) |
| 1491 | GGGATCAGGCTTCCACTTTGGAAACACAGC (SEQ | RTT*1, 2, 3, 4, 7, 10 (GG-to-GT; GGG-to- |
| | ID NO: 1491) | GTG PAM silencing edit) |
| 1492 | GGGATCAGGCTTCCACTTTGGAAACCCAGC (SEQ | RTT*1, 2, 3, 10 |
| | ID NO: 1492) | |
| 1493 | GGGATCAGGCTTCCACTTTGGAAACGCAGC (SEQ | RTT*1, 2, 3, 5, 8, 10 (GG-to-GC;GGG-to- |
| | ID NO: 1493) | GCG PAM silencing edit) |
| 1494 | GGGATCAGGCTTCCACTTTGGAAACTCAGC (SEQ | RTT*1, 2, 3, 6, 9, 10 (GG-to-GA;GGG-to- |
| | ID NO: 1494) | GAG PAM silencing edit) |
| 1495 | TGGGATCAGGCTTCCACTTTGGAAACACAGC | RTT*1, 2, 3, 4, 7, 10 (GG-to-GT;GGG-to- |
| | (SEQ ID NO: 1495) | GTG PAM silencing edit) |
| 1496 | TGGGATCAGGCTTCCACTTTGGAAACCCAGC | RTT*1, 2, 3, 10 |
| | (SEQ ID NO: 1496) | |
| 1497 | TGGGATCAGGCTTCCACTTTGGAAACGCAGC | RTT*1, 2, 3, 5, 8, 10 (GG-to-GC;GGG-to- |
| | (SEQ ID NO: 1497) | GCG PAM silencing edit) |
| 1498 | TGGGATCAGGCTTCCACTTTGGAAACTCAGC | RTT*1, 2, 3, 6, 9, 10 (GG-to-GA;GGG-to- |
| | (SEQ ID NO: 1498) | GAG PAM silencing edit) |
| 1499 | ATGGGATCAGGCTTCCACTTTGGAAACACAGC | RTT*1, 2, 3, 4, 7, 10 (GG-to-GT;GGG-to- |
| | (SEQ ID NO: 1499) | GTG PAM silencing edit) |
| 1500 | ATGGGATCAGGCTTCCACTTTGGAAACCCAGC | RTT*1, 2, 3, 10 |
| | (SEQ ID NO: 1500) | |
| 1501 | ATGGGATCAGGCTTCCACTTTGGAAACGCAGC | RTT*1, 2, 3, 5, 8, 10 (GG-to-GC;GGG-to- |
| | (SEQ ID NO: 1501) | GCG PAM silencing edit) |
| 1502 | ATGGGATCAGGCTTCCACTTTGGAAACTCAGC | RTT*1, 2, 3, 6, 9, 10 (GG-to-GA;GGG-to- |
| | (SEQ ID NO: 1502) | GAG PAM silencing edit) |
| 1503 | TATGGGATCAGGCTTCCACTTTGGAAACACAGC | RTT*1, 2, 3, 4, 7, 10 (GG-to-GT;GGG-to-GTG |
| | (SEQ ID NO: 1503) | PAM silencing edit) |
| 1504 | TATGGGATCAGGCTTCCACTTTGGAAACCCAGC | RTT*1, 2, 3, 10 |
| | (SEQ ID NO: 1504) | |
| 1505 | TATGGGATCAGGCTTCCACTTTGGAAACGCAGC | RTT*1, 2, 3, 5, 8, 10 (GG-to-GC;GGG-to-GCG |
| | (SEQ ID NO: 1505) | PAM silencing edit) |
| 1506 | TATGGGATCAGGCTTCCACTTTGGAAACTCAGC | RTT*1, 2, 3, 6, 9, 10 (GG-to-GA;GGG-to-GAG |
| | (SEQ ID NO: 1506) | PAM silencing edit) |
| 1507 | GTATGGGATCAGGCTTCCACTTTGGAAACACAGC | RTT*1, 2, 3, 4, 7, 10 (GG-to-GT;GGG-to-GTG |
| | (SEQ ID NO: 1507) | PAM silencing edit) |
| 1508 | GTATGGGATCAGGCTTCCACTTTGGAAACCCAGC | RTT*1, 2, 3, 10 |
| | (SEQ ID NO: 1508) | |
| 1509 | GTATGGGATCAGGCTTCCACTTTGGAAACGCAGC | RTT*1, 2, 3, 5, 8, 10 (GG-to-GC;GGG-to-GCG |
| | (SEQ ID NO: 1509) | PAM silencing edit) |
| 1510 | GTATGGGATCAGGCTTCCACTTTGGAAACTCAGC | RTT*1, 2, 3, 6, 9, 10 (GG-to-GA;GGG-to-GAG |
| | (SEQ ID NO: 1510) | PAM silencing edit) |
| 1511 | TGTATGGGATCAGGCTTCCACTTTGGAAACACAG | RTT*1, 2, 3, 4, 7, 10 (GG-to-GT;GGG-to-GTG |
| | C (SEQ ID NO: 1511) | PAM silencing edit) |
| 1512 | TGTATGGGATCAGGCTTCCACTTTGGAAACCCAG | RTT*1, 2, 3, 10 |
| | C (SEQ ID NO: 1512) | |
| 1513 | TGTATGGGATCAGGCTTCCACTTTGGAAACGCAG | RTT*1, 2, 3, 5, 8, 10 (GG-to-GC;GGG-to-GCG |
| | C (SEQ ID NO: 1513) | PAM silencing edit) |
| 1514 | TGTATGGGATCAGGCTTCCACTTTGGAAACTCAG | RTT*1, 2, 3, 6, 9, 10 (GG-to-GA;GGG-to-GAG |
| | C (SEQ ID NO: 1514) | PAM silencing edit) |
| 1515 | CTGTATGGGATCAGGCTTCCACTTTGGAAACACA | RTT*1, 2, 3, 4, 7, 10 (GG-to-GT;GGG-to-GTG |
| | GC (SEQ ID NO: 1515) | PAM silencing edit) |
| 1516 | CTGTATGGGATCAGGCTTCCACTTTGGAAACCCA | RTT*1, 2, 3, 10 |
| | GC (SEQ ID NO: 1516) | |
| 1517 | CTGTATGGGATCAGGCTTCCACTTTGGAAACGCA | RTT*1, 2, 3, 5, 8, 10 (GG-to-GC;GGG-to-GCG |
| | GC (SEQ ID NO: 1517) | PAM silencing edit) |
| 1518 | CTGTATGGGATCAGGCTTCCACTTTGGAAACTCA | RTT*1, 2, 3, 6, 9, 10 (GG-to-GA;GGG-to-GAG |
| | GC (SEQ ID NO: 1518) | PAM silencing edit) |
| 1519 | TCTGTATGGGATCAGGCTTCCACTTTGGAAACAC | RTT*1, 2, 3, 4, 7, 10 (GG-to-GT;GGG-to-GTG |
| | AGC (SEQ ID NO: 1519) | PAM silencing edit) |
| 1520 | TCTGTATGGGATCAGGCTTCCACTTTGGAAACCC | RTT*1, 2, 3, 10 |
| | AGC (SEQ ID NO: 1520) | |
| 1521 | TCTGTATGGGATCAGGCTTCCACTTTGGAAACGC | RTT*1, 2, 3, 5, 8, 10 (GG-to-GC;GGG-to-GCG |
| | AGC (SEQ ID NO: 1521) | PAM silencing edit) |
| 1522 | TCTGTATGGGATCAGGCTTCCACTTTGGAAACTC | RTT*1, 2, 3, 6, 9, 10 (GG-to-GA;GGG-to-GAG |
| | AGC (SEQ ID NO: 1522) | PAM silencing edit) |
| 1523 | TTCTGTATGGGATCAGGCTTCCACTTTGGAAACA | RTT*1, 2, 3, 4, 7, 10 (GG-to-GT;GGG-to-GTG |
| | CAGC (SEQ ID NO: 1523) | PAM silencing edit) |
| 1524 | TTCTGTATGGGATCAGGCTTCCACTTTGGAAACC | RTT*1, 2, 3, 10 |
| | CAGC (SEQ ID NO: 1524) | |
| 1525 | TTCTGTATGGGATCAGGCTTCCACTTTGGAAACG | RTT*1, 2, 3, 5, 8, 10 (GG-to-GC;GGG-to-GCG |
| | CAGC (SEQ ID NO: 1525) | PAM silencing edit) |
| 1526 | TTCTGTATGGGATCAGGCTTCCACTTTGGAAACT | RTT*1, 2, 3, 6, 9, 10 (GG-to-GA;GGG-to-GAG |
| | CAGC (SEQ ID NO: 1526) | PAM silencing edit) |
| 1527 | TTTCTGTATGGGATCAGGCTTCCACTTTGGAAAC | RTT*1, 2, 3, 4, 7, 10 (GG-to-GT;GGG-to-GTG |
| | ACAGC (SEQ ID NO: 1527) | PAM silencing edit) |
| 1528 | TTTCTGTATGGGATCAGGCTTCCACTTTGGAAAC | RTT*1, 2, 3, 10 |
| | CCAGC (SEQ ID NO: 1528) | |
| 1529 | TTTCTGTATGGGATCAGGCTTCCACTTTGGAAAC | RTT*1, 2, 3, 5, 8, 10 (GG-to-GC;GGG-to-GCG |
| | GCAGC (SEQ ID NO: 1529) | PAM silencing edit) |
| 1530 | TTTCTGTATGGGATCAGGCTTCCACTTTGGAAAC | RTT*1, 2, 3, 6, 9, 10 (GG-to-GA;GGG-to-GAG |
| | TCAGC (SEQ ID NO: 1530) | PAM silencing edit) |
| 1531 | CTTTCTGTATGGGATCAGGCTTCCACTTTGGAAA | RTT*1, 2, 3, 4, 7, 10 (GG-to-GT;GGG-to-GTG |
| | CACAGC (SEQ ID NO: 1531) | PAM silencing edit) |
| 1532 | CTTTCTGTATGGGATCAGGCTTCCACTTTGGAAA | RTT*1, 2, 3, 10 |
| | CCCAGC (SEQ ID NO: 1532) | |
| 1533 | CTTTCTGTATGGGATCAGGCTTCCACTTTGGAAA | RTT*1, 2, 3, 5, 8, 10 (GG-to-GC;GGG-to-GCG |
| | CGCAGC (SEQ ID NO: 1533) | PAM silencing edit) |
| 1534 | CTTTCTGTATGGGATCAGGCTTCCACTTTGGAAA | RTT*1, 2, 3, 6, 9, 10 (GG-to-GA;GGG-to-GAG |
| | CTCAGC (SEQ ID NO: 1534) | PAM silencing edit) |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b*1 ngRNA spacer (GG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b*2 ngRNA spacer (TG;TGG PAM) |
| 29 | CCTTTTCGCTTTTTCCAAGA (SEQ ID NO: 29) | PE3 ngRNA spacer (TG;TGG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b*3 ngRNA spacer (AG;AGG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG;TGG PAM) |
| 1535 | GGCTTCCACTTTGGAAACAC (SEQ ID NO: 1535) | PE3b*4 ngRNA spacer (AG PAM) |
| 36 | GGCTTCCACTTTGGAAACCC (SEQ ID NO: 36) | PE3 ngRNA spacer (AG PAM) |
| 1536 | GGCTTCCACTTTGGAAACGC (SEQ ID NO: 1536) | PE3b*5 ngRNA spacer (AG PAM) |
| 1537 | GGCTTCCACTTTGGAAACTC (SEQ ID NO: 1537) | PE3b*6 ngRNA spacer (AG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 1538 | TCCACTTTGGAAACACAGCA (SEQ ID NO: 1538) | PE3b*7 ngRNA spacer (AG PAM) |
| 40 | TCCACTTTGGAAACCCAGCA (SEQ ID NO: 40) | PE3 ngRNA spacer (AG PAM) |
| 1539 | TCCACTTTGGAAACGCAGCA (SEQ ID NO: 1539) | PE3b*8 ngRNA spacer (AG PAM) |
| 1540 | TCCACTTTGGAAACTCAGCA (SEQ ID NO: 1540) | PE3b*9 ngRNA spacer (AG PAM) |
| 41 | TGATTATCAGTTTTGGATGC (SEQ ID NO: 41) | PE3 ngRNA spacer (AG PAM) |
| 43 | TGGGATCAGGCTTCCACTTT (SEQ ID NO: 43) | PE3b*10 ngRNA spacer (GG PAM) |
| 44 | TTATCAGTTTTGGATGCAGA (SEQ ID NO: 44) | PE3 ngRNA spacer (AG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG;TGG PAM) |
| 48 | TTTTGGATGCAGAAGCTTTC (SEQ ID NO: 48) | PE3 ngRNA spacer (TG PAM) |
| 1541 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CC (SEQ ID NO: 1541) | |
| 1542 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTG (SEQ ID NO: 1542) | |
| 1543 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACC (SEQ ID NO: 1543) | |
| 1544 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTC (SEQ ID NO: 1544) | |
| 1545 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTG (SEQ ID NO: 1545) | |
| 1546 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTTTT (SEQ ID NO: 1546) | |
| 1547 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACC (SEQ ID NO: 1547) | |
| 1548 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTCTT (SEQ ID NO: 1548) | |
| 1549 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTC (SEQ ID NO: 1549) | |
| 1550 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTTTT (SEQ ID NO: 1550) | |
| 1551 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTTTT (SEQ ID NO: 1551) | |
| 1552 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTCT (SEQ ID NO: 1552) | |
| 1553 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTG (SEQ ID NO: 1553) | |
| 1554 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACC (SEQ ID NO: 1554) | |
| 1555 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTCTTTT (SEQ ID NO: 1555) | |
| 1556 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTTTT (SEQ ID NO: 1556) | |
| 1557 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTC (SEQ ID NO: 1557) | |
| 1558 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTG (SEQ ID NO: 1558) | |
| 1559 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTTTT (SEQ ID NO: 1559) | |
| 1560 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCATGGG | |
| | ATCAGGCTTCCACTTTGGAAACCCAGCAAGACA | |
| | CCTGTCTTTTTT (SEQ ID NO: 1560) | |
| 1561 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTCTTTT (SEQ ID NO: 1561) | |
| 1562 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTCTT (SEQ ID NO: 1562) | |
| 1563 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTC (SEQ ID NO: 1563) | |
| 1564 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCGTATG | |
| | GGATCAGGCTTCCACTTTGGAAACCCAGCAAGA | |
| | CACCTGTCTTTTT (SEQ ID NO: 1564) | |
| 1565 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTTTT (SEQ ID NO: 1565) | |
| 1566 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTTTT (SEQ ID NO: 1566) | |
| 1567 | GTGAAGACAGGTGTCTTGCTGTTTTAGAGCTAGA | pegRNA*1, 2, 3, 10 |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTCTT (SEQ ID NO: 1567) | |
| 1568 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTCTTTT (SEQ ID NO: 1568) | |
| 1569 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTTTT (SEQ ID NO: 1569) | |
| 1570 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTCTGTA | |
| | TGGGATCAGGCTTCCACTTTGGAAACCCAGCAA | |
| | GACACCTGTCTTTTTT (SEQ ID NO: 1570) | |
| 1571 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTCTTTT (SEQ ID NO: 1571) | |
| 1572 | GTGAAGACAGGTGTCTTGCTGTTTTAGAGCTAGA | pegRNA*1, 2, 3, 10, contains 3′ TTTT |
| | AATAGCAAGTTAAAATAAGGCTAGTCCGTTATCA | |
| | ACTTGAAAAAGTGGCACCGAGTCGGTGCTTTCTG | |
| | TATGGGATCAGGCTTCCACTTTGGAAACCCAGCA | |
| | AGACACCTGTCTTTTTT (SEQ ID NO: 1572) | |
| 1573 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCATGGG | 3′ TTTT |
| | ATCAGGCTTCCACTTTGGAAACCCAGCAAGACA | |
| | CCAACATTGACGCGTCTCTACGTGGGGGCGCGTT | |
| | TT (SEQ ID NO: 1573) | |
| 1574 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCATGGG | 3′ TTTT |
| | ATCAGGCTTCCACTTTGGAAACCCAGCAAGACA | |
| | CCTGAACATTGACGCGTCTCTACGTGGGGGCGCG | |
| | TTTT (SEQ ID NO: 1574) | |
| 1575 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCGTATG | 3′ TTTT |
| | GGATCAGGCTTCCACTTTGGAAACCCAGCAAGA | |
| | CACCAACATTGACGCGTCTCTACGTGGGGGCGC | |
| | GTTTT (SEQ ID NO: 1575) | |
| 1576 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCATGGG | 3′ TTTT |
| | ATCAGGCTTCCACTTTGGAAACCCAGCAAGACA | |
| | CCTGTCAACATTGACGCGTCTCTACGTGGGGGCG | |
| | CGTTTT (SEQ ID NO: 1576) | |
| 1577 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCGTATG | 3′ TTTT |
| | GGATCAGGCTTCCACTTTGGAAACCCAGCAAGA | |
| | CACCTGAACATTGACGCGTCTCTACGTGGGGGCG | |
| | CGTTTT (SEQ ID NO: 1577) | |
| 1578 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTCTGTA | 3′ TTTT |
| | TGGGATCAGGCTTCCACTTTGGAAACCCAGCAA | |
| | GACACCAACATTGACGCGTCTCTACGTGGGGGC | |
| | GCGTTTT (SEQ ID NO: 1578) | |
| 1579 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCATGGG | 3′ TTTT |
| | ATCAGGCTTCCACTTTGGAAACCCAGCAAGACA | |
| | CCTGTCTTAACATTGACGCGTCTCTACGTGGGGG | |
| | CGCGTTTT (SEQ ID NO: 1579) | |
| 1580 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCGTATG | 3′ TTTT |
| | GGATCAGGCTTCCACTTTGGAAACCCAGCAAGA | |
| | CACCTGTCAACATTGACGCGTCTCTACGTGGGGG | |
| | CGCGTTTT (SEQ ID NO: 1580) | |
| 1581 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCGTATG | 3′ TTTT |
| | GGATCAGGCTTCCACTTTGGAAACCCAGCAAGA | |
| | CACCTGTCTAACATTGACGCGTCTCTACGTGGGG | |
| | GCGCGTTTT (SEQ ID NO: 1581) | |
| 1582 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA ;hairpin SEQ ID NO: 3591; |
| | ACTTGAAAAAGTGGCACCGAGTCGGTGCTCTGTA | 3′ TTTT |
| | TGGGATCAGGCTTCCACTTTGGAAACCCAGCAA | |
| | GACACCTGAACATTGACGCGTCTCTACGTGGGG | |
| | GCGCGTTTT (SEQ ID NO: 1582) | |
| 1583 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTTTCTG | 3′ TTTT |
| | TATGGGATCAGGCTTCCACTTTGGAAACCCAGCA | |
| | AGACACCAACATTGACGCGTCTCTACGTGGGGG | |
| | CGCGTTTT (SEQ ID NO: 1583) | |
| 1584 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTCTGTA | 3′ TTTT |
| | TGGGATCAGGCTTCCACTTTGGAAACCCAGCAA | |
| | GACACCTGTCAACATTGACGCGTCTCTACGTGGG | |
| | GGCGCGTTTT (SEQ ID NO: 1584) | |
| 1585 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTTTCTG | 3′ TTTT |
| | TATGGGATCAGGCTTCCACTTTGGAAACCCAGCA | |
| | AGACACCTGAACATTGACGCGTCTCTACGTGGG | |
| | GGCGCGTTTT (SEQ ID NO: 1585) | |
| 1586 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTCTGTA | 3′ TTTT |
| | TGGGATCAGGCTTCCACTTTGGAAACCCAGCAA | |
| | GACACCTGTCTTAACATTGACGCGTCTCTACGTG | |
| | GGGGCGCGTTTT (SEQ ID NO: 1586) | |
| 1587 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTTTCTG | 3′ TTTT |
| | TATGGGATCAGGCTTCCACTTTGGAAACCCAGCA | |
| | AGACACCTGTCAACATTGACGCGTCTCTACGTGG | |
| | GGGCGCGTTTT (SEQ ID NO: 1587) | |
| 1588 | GTGAAGACAGGTGTCTTGCTGTTTTAGAGCTAGA | pegRNA*1, 2, 3, 10, contains linker |
| | AATAGCAAGTTAAAATAAGGCTAGTCCGTTATCA | sequence AACATTGA; hairpin SEQ ID NO: 3591; |
| | ACTTGAAAAAGTGGCACCGAGTCGGTGCTTTCTG | 3′ TTTT |
| | TATGGGATCAGGCTTCCACTTTGGAAACCCAGCA | |
| | AGACACCTGTCTTAACATTGACGCGTCTCTACGT | |
| | GGGGGCGCGTTTT (SEQ ID NO: 1588) | |
| 49 | TTTCTTGTGATTATCAGTTTGTTTTAGAGCTAGAA | PE3 ngRNA (TG; TGG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b*3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b*2 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG; TGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b*3 ngRNA (AG; AGG PAM), contains 3′ |
| | AATAGCAAGTTAAAATAAGGCTAGTCCGTTATCA | TTTT |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG; TGG PAM), contains 3′ |
| | AATAGCAAGTTAAAATAAGGCTAGTCCGTTATCA | TTTT; 5′G |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b*2 ngRNA (TG; TGG PAM), contains 3′ |
| | AAATAGCAAGTTAAAATAAGGCTAGTCCGTTATC | TTTT; 5′G |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b*3 ngRNA (AG; AGG PAM), contains |
| | AATAGCAAGTTAAAATAAGGCTAGTCCGTTATCA | linker sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT; 5′G |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b*2 ngRNA (TG; TGG PAM), contains |
| | AAATAGCAAGTTAAAATAAGGCTAGTCCGTTATC | linker sequence AACATTGA; hairpin SEQ ID |
| | AACTTGAAAAAGTGGCACCGAGTCGGTGCAACA | NO: 3591; 3′ TTTT; 5′G |
| | TTGACGCGTCTCTACGTGGGGGCGCGTTTT (SEQ | |
| | ID NO: 60) | |
| 3651 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCATGGG | |
| | ATCAGGCTTCCACTTTGGAAACCCAGCAAGACA | |
| | CCTGAACATTGACGCGTCTCTACGTGGGGGCGCG | |
| | (SEQ ID NO: 3651) | |
| 3652 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTATG | |
| | GGATCAGGCTTCCACTTTGGAAACCCAGCAAGA | |
| | CACCAACATTGACGCGTCTCTACGTGGGGGCGC | |
| | G (SEQ ID NO: 3652) | |
| 3653 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCATGGG | |
| | ATCAGGCTTCCACTTTGGAAACCCAGCAAGACA | |
| | CCTGTCAACATTGACGCGTCTCTACGTGGGGGCG | |
| | CG (SEQ ID NO: 3653) | |
| 3654 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTATG | |
| | GGATCAGGCTTCCACTTTGGAAACCCAGCAAGA | |
| | CACCTGAACATTGACGCGTCTCTACGTGGGGGCG | |
| | CG (SEQ ID NO: 3654) | |
| 3655 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTCTGTA | |
| | TGGGATCAGGCTTCCACTTTGGAAACCCAGCAA | |
| | GACACCAACATTGACGCGTCTCTACGTGGGGGC | |
| | GCG (SEQ ID NO: 3655) | |
| 3656 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCATGGG | |
| | ATCAGGCTTCCACTTTGGAAACCCAGCAAGACA | |
| | CCTGTCTTAACATTGACGCGTCTCTACGTGGGGG | |
| | CGCG (SEQ ID NO: 3656) | |
| 3657 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTATG | |
| | GGATCAGGCTTCCACTTTGGAAACCCAGCAAGA | |
| | CACCTGTCAACATTGACGCGTCTCTACGTGGGGG | |
| | CGCG (SEQ ID NO: 3657) | |
| 3658 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCGTATG | |
| | GGATCAGGCTTCCACTTTGGAAACCCAGCAAGA | |
| | CACCTGTCTAACATTGACGCGTCTCTACGTGGGG | |
| | GCGCG (SEQ ID NO: 3658) | |
| 3659 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTCTGTA | |
| | TGGGATCAGGCTTCCACTTTGGAAACCCAGCAA | |
| | GACACCTGAACATTGACGCGTCTCTACGTGGGG | |
| | GCGCG (SEQ ID NO: 3659) | |
| 3660 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTTTCTG | |
| | TATGGGATCAGGCTTCCACTTTGGAAACCCAGCA | |
| | AGACACCAACATTGACGCGTCTCTACGTGGGGG | |
| | CGCG (SEQ ID NO: 3660) | |
| 3661 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTCTGTA | |
| | TGGGATCAGGCTTCCACTTTGGAAACCCAGCAA | |
| | GACACCTGTCAACATTGACGCGTCTCTACGTGGG | |
| | GGCGCG (SEQ ID NO: 3661) | |
| 3662 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTTTCTG | |
| | TATGGGATCAGGCTTCCACTTTGGAAACCCAGCA | |
| | AGACACCTGAACATTGACGCGTCTCTACGTGGG | |
| | GGCGCG (SEQ ID NO: 3662) | |
| 3663 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTCTGTA | |
| | TGGGATCAGGCTTCCACTTTGGAAACCCAGCAA | |
| | GACACCTGTCTTAACATTGACGCGTCTCTACGTG | |
| | GGGGCGCG (SEQ ID NO: 3663) | |
| 3664 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTTTCTG | |
| | TATGGGATCAGGCTTCCACTTTGGAAACCCAGCA | |
| | AGACACCTGTCAACATTGACGCGTCTCTACGTGG | |
| | GGGCGCG (SEQ ID NO: 3664) | |
| 3665 | GTGAAGACAGGTGTCTTGCTGTTTTAGAGCTAGA | pegRNA*1, 2, 3, 10, contains linker sequence |
| | AATAGCAAGTTAAAATAAGGCTAGTCCGTTATCA | AACATTGA; hairpin SEQ ID NO: 3591 |
| | ACTTGAAAAAGTGGCACCGAGTCGGTGCTTTCTG | |
| | TATGGGATCAGGCTTCCACTTTGGAAACCCAGCA | |
| | AGACACCTGTCTTAACATTGACGCGTCTCTACGT | |
| | GGGGGCGCG (SEQ ID NO: 3665) | |

TABLE 20

| Sequence | | |
|---|---|---|
| Number | Sequence | Description |
| 1589 | ATTATCAGTTTTGGATG (SEQ ID NO: 1589) | PEgRNA spacer (CAG PAM) |
| 1590 | GATTATCAGTTTTGGATG (SEQ ID NO: 1590) | PEgRNA spacer (CAG PAM) |
| 1591 | TGATTATCAGTTTTGGATG (SEQ ID NO: 1591) | PEgRNA spacer (CAG PAM) |
| 1226 | GTGATTATCAGTTTTGGATG (SEQ ID NO: 1226) | PEgRNA spacer (CAG PAM) |
| 1592 | TGTGATTATCAGTTTTGGATG (SEQ ID NO: 1592) | PEgRNA spacer (CAG PAM) |
| 1593 | TTGTGATTATCAGTTTTGGATG (SEQ ID NO: 1593) | PEgRNA spacer (CAG PAM) |
| 1594 | CCAAA | PBS |
| 1595 | CCAAAA | PBS |
| 1596 | CCAAAAC | PBS |
| 1597 | CCAAAACT | PBS |
| 1598 | CCAAAACTG | PBS |
| 1599 | CCAAAACTGA (SEQ ID NO: 1599) | PBS |
| 1600 | CCAAAACTGAT (SEQ ID NO: 1600) | PBS |
| 1601 | CCAAAACTGATA (SEQ ID NO: 1601) | PBS |
| 1602 | CCAAAACTGATAA (SEQ ID NO: 1602) | PBS |
| 1603 | CCAAAACTGATAAT (SEQ ID NO: 1603) | PBS |
| 1604 | CCAAAACTGATAATC (SEQ ID NO: 1604) | PBS |
| 1605 | CCAAAACTGATAATCA (SEQ ID NO: 1605) | PBS |
| 1606 | CCAAAACTGATAATCAC (SEQ ID NO: 1606) | PBS |
| 1607 | CCAAAACTGATAATCACA (SEQ ID NO: 1607) | PBS |
| 1608 | CCAAAACTGATAATCACAA (SEQ ID NO: 1608) | PBS |
| 1609 | CCCATACAGAAAGCTTCTGCAT (SEQ ID NO: 1609) | RTT |
| 1610 | CCCATACAGAAAGCTTTTGCAT (SEQ ID NO: 1610) | RTT (CAG-to-CAA PAM silencing edit) |
| 1611 | TCCCATACAGAAAGCTTCTGCAT (SEQ ID NO: | RTT |
| | 1611) | |
| 1612 | TCCCATACAGAAAGCTTTTGCAT (SEQ ID NO: | RTT (CAG-to-CAA PAM silencing edit) |
| | 1612) | |
| 1613 | ATCCCATACAGAAAGCTTCTGCAT (SEQ ID NO: | RTT |
| | 1613) | |
| 1614 | ATCCCATACAGAAAGCTTTTGCAT (SEQ ID NO: | RTT (CAG-to-CAA PAM silencing edit) |
| | 1614) | |
| 1615 | GATCCCATACAGAAAGCTTCTGCAT (SEQ ID NO: | RTT |
| | 1615) | |
| 1616 | GATCCCATACAGAAAGCTTTTGCAT (SEQ ID NO: | RTT (CAG-to-CAA PAM silencing edit) |
| | 1616) | |
| 1617 | TGATCCCATACAGAAAGCTTCTGCAT (SEQ ID | RTT |
| | NO: 1617) | |
| 1618 | TGATCCCATACAGAAAGCTTTTGCAT (SEQ ID | RTT (CAG-to-CAA PAM silencing edit) |
| | NO: 1618) | |
| 1619 | CTGATCCCATACAGAAAGCTTCTGCAT (SEQ ID | RTT |
| | NO: 1619) | |
| 1620 | CTGATCCCATACAGAAAGCTTTTGCAT (SEQ ID | RTT (CAG-to-CAA PAM silencing edit) |
| | NO: 1620) | |
| 1621 | CCTGATCCCATACAGAAAGCTTCTGCAT (SEQ ID | RTT |
| | NO: 1621) | |
| 1622 | CCTGATCCCATACAGAAAGCTTTTGCAT (SEQ ID | RTT (CAG-to-CAA PAM silencing edit) |
| | NO: 1622) | |
| 1623 | GCCTGATCCCATACAGAAAGCTTCTGCAT (SEQ | RTT |
| | ID NO: 1623) | |
| 1624 | GCCTGATCCCATACAGAAAGCTTTTGCAT (SEQ | RTT (CAG-to-CAA PAM silencing edit) |
| | ID NO: 1624) | |
| 1625 | AGCCTGATCCCATACAGAAAGCTTCTGCAT (SEQ | RTT |
| | ID NO: 1625) | |
| 1626 | AGCCTGATCCCATACAGAAAGCTTTTGCAT (SEQ | RTT (CAG-to-CAA PAM silencing edit) |
| | ID NO: 1626) | |
| 1627 | AAGCCTGATCCCATACAGAAAGCTTCTGCAT | RTT |
| | (SEQ ID NO: 1627) | |
| 1628 | AAGCCTGATCCCATACAGAAAGCTTTTGCAT | RTT (CAG-to-CAA PAM silencing edit) |
| | (SEQ ID NO: 1628) | |
| 1629 | GAAGCCTGATCCCATACAGAAAGCTTCTGCAT | RTT |
| | (SEQ ID NO: 1629) | |
| 1630 | GAAGCCTGATCCCATACAGAAAGCTTTTGCAT | RTT (CAG-to-CAA PAM silencing edit) |
| | (SEQ ID NO: 1630) | |
| 1631 | GGAAGCCTGATCCCATACAGAAAGCTTCTGCAT | RTT |
| | (SEQ ID NO: 1631) | |
| 1632 | GGAAGCCTGATCCCATACAGAAAGCTTTTGCAT | RTT (CAG-to-CAA PAM silencing edit) |
| | (SEQ ID NO: 1632) | |
| 1633 | TGGAAGCCTGATCCCATACAGAAAGCTTCTGCAT | RTT |
| | (SEQ ID NO: 1633) | |
| 1634 | TGGAAGCCTGATCCCATACAGAAAGCTTTTGCAT | RTT (CAG-to-CAA PAM silencing edit) |
| | (SEQ ID NO: 1634) | |
| 1635 | GTGGAAGCCTGATCCCATACAGAAAGCTTCTGC | RTT |
| | AT (SEQ ID NO: 1635) | |
| 1636 | GTGGAAGCCTGATCCCATACAGAAAGCTTTTGCA | RTT (CAG-to-CAA PAM silencing edit) |
| | T (SEQ ID NO: 1636) | |
| 1637 | AGTGGAAGCCTGATCCCATACAGAAAGCTTCTG | RTT |
| | CAT (SEQ ID NO: 1637) | |
| 1638 | AGTGGAAGCCTGATCCCATACAGAAAGCTTTTGC | RTT (CAG-to-CAA PAM silencing edit) |
| | AT (SEQ ID NO: 1638) | |
| 1639 | AAGTGGAAGCCTGATCCCATACAGAAAGCTTCT | RTT |
| | GCAT (SEQ ID NO: 1639) | |
| 1640 | AAGTGGAAGCCTGATCCCATACAGAAAGCTTTTG | RTT (CAG-to-CAA PAM silencing edit) |
| | CAT (SEQ ID NO: 1640) | |
| 1641 | AAAGTGGAAGCCTGATCCCATACAGAAAGCTTC | RTT |
| | TGCAT (SEQ ID NO: 1641) | |
| 1642 | AAAGTGGAAGCCTGATCCCATACAGAAAGCTTTT | RTT (CAG-to-CAA PAM silencing edit) |
| | GCAT (SEQ ID NO: 1642) | |
| 1643 | CAAAGTGGAAGCCTGATCCCATACAGAAAGCTT | RTT |
| | CTGCAT (SEQ ID NO: 1643) | |
| 1644 | CAAAGTGGAAGCCTGATCCCATACAGAAAGCTT | RTT (CAG-to-CAA PAM silencing edit) |
| | TTGCAT (SEQ ID NO: 1644) | |
| 1645 | CCAAAGTGGAAGCCTGATCCCATACAGAAAGCT | RTT |
| | TCTGCAT (SEQ ID NO: 1645) | |
| 1646 | CCAAAGTGGAAGCCTGATCCCATACAGAAAGCT | RTT (CAG-to-CAA PAM silencing edit) |
| | TTTGCAT (SEQ ID NO: 1646) | |
| 480 | AAGGCTTCATACATCTTCCT (SEQ ID NO: 480) | PE3 ngRNA spacer (TAG PAM) |
| 481 | AGTGGAAGCCTGATCCCATA (SEQ ID NO: 481) | PE3b ngRNA spacer (CAG PAM) |
| 482 | CACTACTTACCATCTCTTTC (SEQ ID NO: 482) | PE3 ngRNA spacer (AAG PAM) |
| 483 | CCTTAGGAACTCCTGGAACT (SEQ ID NO: 483) | PE3 ngRNA spacer (GAG PAM) |
| 484 | GAAGCCTGATCCCATACAGA (SEQ ID NO: 484) | PE3b ngRNA spacer (AAG PAM) |
| 485 | GGAACTGAGCCACGTGAAGA (SEQ ID NO: 485) | PE3 ngRNA spacer (CAG PAM) |
| 486 | GGTGTCTTGCTGGGTTTCCA (SEQ ID NO: 486) | PE3 ngRNA spacer (AAG PAM) |
| 487 | TCCTGGAACTGAGCCACGTG (SEQ ID NO: 487) | PE3 ngRNA spacer (AAG PAM) |
| 488 | TTGCTGGGTTTCCAAAGTGG (SEQ ID NO: 488) | PE3 ngRNA spacer (AAG PAM) |

TABLE 21

| Sequence | | |
|---|---|---|
| Number | Sequence | Description |
| 1647 | TCTTGCTGGGTTTCCAA (SEQ ID NO: 1647) | PEgRNA spacer (AG PAM) |
| 1648 | GTCTTGCTGGGTTTCCAA (SEQ ID NO: 1648) | PEgRNA spacer (AG PAM) |
| 1649 | TGTCTTGCTGGGTTTCCAA (SEQ ID NO: 1649) | PEgRNA spacer (AG PAM) |
| 214 | GTGTCTTGCTGGGTTTCCAA (SEQ ID NO: 214) | PERNA spacer (AG PAM) |
| 1650 | GGTGTCTTGCTGGGTTTCCAA (SEQ ID NO: 1650) | PEgRNA spacer (AG PAM) |
| 1651 | AGGTGTCTTGCTGGGTTTCCAA (SEQ ID NO: 1651) | PEgRNA spacer (AG PAM) |
| 1652 | GAAAC | PBS |
| 1653 | GAAACC | PBS |
| 1654 | GAAACCC | PBS |
| 1655 | GAAACCCA | PBS |
| 1656 | GAAACCCAG | PBS |
| 1657 | GAAACCCAGC (SEQ ID NO: 1657) | PBS |
| 1658 | GAAACCCAGCA (SEQ ID NO: 1658) | PBS |
| 1659 | GAAACCCAGCAA (SEQ ID NO: 1659) | PBS |
| 1660 | GAAACCCAGCAAG (SEQ ID NO: 1660) | PBS |
| 1661 | GAAACCCAGCAAGA (SEQ ID NO: 1661) | PBS |
| 1662 | GAAACCCAGCAAGAC (SEQ ID NO: 1662) | PBS |
| 1663 | GAAACCCAGCAAGACA (SEQ ID NO: 1663) | PBS |
| 1664 | GAAACCCAGCAAGACAC (SEQ ID NO: 1664) | PBS |
| 1665 | GAAACCCAGCAAGACACC (SEQ ID NO: 1665) | PBS |
| 1666 | GAAACCCAGCAAGACACCT (SEQ ID NO: 1666) | PBS |
| 1667 | GATCAGGCTTCCACTTTG (SEQ ID NO: 1667) | RTT |
| 1668 | GGATCAGGCTTCCACTTTG (SEQ ID NO: 1668) | RTT |
| 1669 | GGGATCAGGCTTCCACTTTG (SEQ ID NO: 1669) | RTT |
| 1670 | TGGGATCAGGCTTCCACTTTG (SEQ ID NO: 1670) | RTT |
| 1671 | ATGGGATCAGGCTTCCACTTTG (SEQ ID NO: 1671) | RTT |
| 1672 | TATGGGATCAGGCTTCCACTTTG (SEQ ID NO: | RTT |
| | 1672) | |
| 1673 | GTATGGGATCAGGCTTCCACTTTG (SEQ ID NO: | RTT |
| | 1673) | |
| 1674 | TGTATGGGATCAGGCTTCCACTTTG (SEQ ID NO: | RTT |
| | 1674) | |
| 1675 | CTGTATGGGATCAGGCTTCCACTTTG (SEQ ID NO: | RTT |
| | 1675) | |
| 1676 | TCTGTATGGGATCAGGCTTCCACTTTG (SEQ ID | RTT |
| | NO: 1676) | |
| 1677 | TTCTGTATGGGATCAGGCTTCCACTTTG (SEQ ID | RTT |
| | NO: 1677) | |
| 1678 | TTTCTGTATGGGATCAGGCTTCCACTTTG (SEQ ID | RTT |
| | NO: 1678) | |
| 1679 | CTTTCTGTATGGGATCAGGCTTCCACTTTG (SEQ | RTT |
| | ID NO: 1679) | |
| 1680 | GCTTTCTGTATGGGATCAGGCTTCCACTTTG (SEQ | RTT |
| | ID NO: 1680) | |
| 1681 | AGCTTTCTGTATGGGATCAGGCTTCCACTTTG | RTT |
| | (SEQ ID NO: 1681) | |
| 1682 | AAGCTTTCTGTATGGGATCAGGCTTCCACTTTG | RTT |
| | (SEQ ID NO: 1682) | |
| 1683 | GAAGCTTTCTGTATGGGATCAGGCTTCCACTTTG | RTT |
| | (SEQ ID NO: 1683) | |
| 1684 | AGAAGCTTTCTGTATGGGATCAGGCTTCCACTTT | RTT |
| | G (SEQ ID NO: 1684) | |
| 1685 | CAGAAGCTTTCTGTATGGGATCAGGCTTCCACTT | RTT |
| | TG (SEQ ID NO: 1685) | |
| 1686 | GCAGAAGCTTTCTGTATGGGATCAGGCTTCCACT | RTT |
| | TTG (SEQ ID NO: 1686) | |
| 1687 | TGCAGAAGCTTTCTGTATGGGATCAGGCTTCCAC | RTT |
| | TTTG (SEQ ID NO: 1687) | |
| 1688 | ATGCAGAAGCTTTCTGTATGGGATCAGGCTTCCA | RTT |
| | CTTTG (SEQ ID NO: 1688) | |
| 1689 | GATGCAGAAGCTTTCTGTATGGGATCAGGCTTCC | RTT |
| | ACTTTG (SEQ ID NO: 1689) | |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b ngRNA spacer (GG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b ngRNA spacer (TG PAM) |
| 1108 | CAGTGCTGGACAGGCTGCTG (SEQ ID NO: 1108) | PE3 ngRNA spacer (TG PAM) |
| 29 | CCTTTTCGCTTTTTCCAAGA (SEQ ID NO: 29) | PE3 ngRNA spacer (TG PAM) |
| 1110 | CTGGACAGGCTGCTGTGAAG (SEQ ID NO: 1110) | PE3 ngRNA spacer (GG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b ngRNA spacer (AG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 1111 | GCTGGACAGGCTGCTGTGAA (SEQ ID NO: 1111) | PE3 ngRNA spacer (GG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 41 | TGATTATCAGTTTTGGATGC (SEQ ID NO: 41) | PE3 ngRNA spacer (AG PAM) |
| 1113 | TGCTGGACAGGCTGCTGTGA (SEQ ID NO: 1113) | PE3 ngRNA spacer (AG PAM) |
| 43 | TGGGATCAGGCTTCCACTTT (SEQ ID NO: 43) | PE3b ngRNA spacer (GG PAM) |
| 44 | TTATCAGTTTTGGATGCAGA (SEQ ID NO: 44) | PE3 ngRNA spacer (AG PAM) |
| 1114 | TTCAGTGCTGGACAGGCTGC (SEQ ID NO: 1114) | PE3 ngRNA spacer (TG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 60) | |

TABLE 22

| Sequence | | |
|---|---|---|
| Number | Sequence | Description |
| 1690 | ATCTTTCTTGTGATTAT (SEQ ID NO: 1690) | PEgRNA spacer (CAG PAM) |
| 1691 | GATCTTTCTTGTGATTAT (SEQ ID NO: 1691) | PEgRNA spacer (CAG PAM) |
| 1692 | AGATCTTTCTTGTGATTAT (SEQ ID NO: 1692) | PEgRNA spacer (CAG PAM) |
| 1228 | TAGATCTTTCTTGTGATTAT (SEQ ID NO: 1228) | PEgRNA spacer (CAG PAM) |
| 1693 | GTAGATCTTTCTTGTGATTAT (SEQ ID NO: 1693) | PEgRNA spacer (CAG PAM) |
| 1694 | AGTAGATCTTTCTTGTGATTAT (SEQ ID NO: 1694) | PEgRNA spacer (CAG PAM) |
| 1695 | ATCAC | PBS |
| 1696 | ATCACA | PBS |
| 1697 | ATCACAA | PBS |
| 1698 | ATCACAAG | PBS |
| 1699 | ATCACAAGA | PBS |
| 1700 | ATCACAAGAA (SEQ ID NO: 1700) | PBS |
| 1701 | ATCACAAGAAA (SEQ ID NO: 1701) | PBS |
| 1702 | ATCACAAGAAAG (SEQ ID NO: 1702) | PBS |
| 1703 | ATCACAAGAAAGA (SEQ ID NO: 1703) | PBS |
| 1704 | ATCACAAGAAAGAT (SEQ ID NO: 1704) | PBS |
| 1705 | ATCACAAGAAAGATC (SEQ ID NO: 1705) | PBS |
| 1706 | ATCACAAGAAAGATCT (SEQ ID NO: 1706) | PBS |
| 1707 | ATCACAAGAAAGATCTA (SEQ ID NO: 1707) | PBS |
| 1708 | ATCACAAGAAAGATCTAC (SEQ ID NO: 1708) | PBS |
| 1709 | ATCACAAGAAAGATCTACT (SEQ ID NO: 1709) | PBS |
| 1710 | CCCATACAGAAAGCTTCTGCATCCAAAACTGATA | RTT |
| | (SEQ ID NO: 1710) | |
| 1711 | CCCATACAGAAAGCTTCTGCATCCAAAATTGATA | RTT (CAG-to-CAA PAM silencing edit) |
| | (SEQ ID NO: 1711) | |
| 1712 | TCCCATACAGAAAGCTTCTGCATCCAAAACTGAT | RTT |
| | A (SEQ ID NO: 1712) | |
| 1713 | TCCCATACAGAAAGCTTCTGCATCCAAAATTGAT | RTT (CAG-to-CAA PAM silencing edit) |
| | A (SEQ ID NO: 1713) | |
| 1714 | ATCCCATACAGAAAGCTTCTGCATCCAAAACTGA | RTT |
| | TA (SEQ ID NO: 1714) | |
| 1715 | ATCCCATACAGAAAGCTTCTGCATCCAAAATTGA | RTT (CAG-to-CAA PAM silencing edit) |
| | TA (SEQ ID NO: 1715) | |
| 1716 | GATCCCATACAGAAAGCTTCTGCATCCAAAACTG | RTT |
| | ATA (SEQ ID NO: 1716) | |
| 1717 | GATCCCATACAGAAAGCTTCTGCATCCAAAATTG | RTT (CAG-to-CAA PAM silencing edit) |
| | ATA (SEQ ID NO: 1717) | |
| 1718 | TGATCCCATACAGAAAGCTTCTGCATCCAAAACT | RTT |
| | GATA (SEQ ID NO: 1718) | |
| 1719 | TGATCCCATACAGAAAGCTTCTGCATCCAAAATT | RTT (CAG-to-CAA PAM silencing edit) |
| | GATA (SEQ ID NO: 1719) | |
| 1720 | CTGATCCCATACAGAAAGCTTCTGCATCCAAAAC | RTT |
| | TGATA (SEQ ID NO: 1720) | |
| 1721 | CTGATCCCATACAGAAAGCTTCTGCATCCAAAAT | RTT (CAG-to-CAA PAM silencing edit) |
| | TGATA (SEQ ID NO: 1721) | |
| 1722 | CCTGATCCCATACAGAAAGCTTCTGCATCCAAAA | RTT |
| | CTGATA (SEQ ID NO: 1722) | |
| 1723 | CCTGATCCCATACAGAAAGCTTCTGCATCCAAAA | RTT (CAG-to-CAA PAM silencing edit) |
| | TTGATA (SEQ ID NO: 1723) | |
| 480 | AAGGCTTCATACATCTTCCT (SEQ ID NO: 480) | PE3 ngRNA spacer (TAG PAM) |
| 481 | AGTGGAAGCCTGATCCCATA (SEQ ID NO: 481) | PE3b ngRNA spacer (CAG PAM) |
| 483 | CCTTAGGAACTCCTGGAACT (SEQ ID NO: 483) | PE3 ngRNA spacer (GAG PAM) |
| 484 | GAAGCCTGATCCCATACAGA (SEQ ID NO: 484) | PE3b ngRNA spacer (AAG PAM) |
| 485 | GGAACTGAGCCACGTGAAGA (SEQ ID NO: 485) | PE3 ngRNA spacer (CAG PAM) |
| 486 | GGTGTCTTGCTGGGTTTCCA (SEQ ID NO: 486) | PE3 ngRNA spacer (AAG PAM) |
| 487 | TCCTGGAACTGAGCCACGTG (SEQ ID NO: 487) | PE3 ngRNA spacer (AAG PAM) |
| 488 | TTGCTGGGTTTCCAAAGTGG (SEQ ID NO: 488) | PE3 ngRNA spacer (AAG PAM) |

TABLE 23

| Sequence | | |
|---|---|---|
| Number | Sequence | Description |
| 1724 | TGCTGGGTTTCCAAAGT (SEQ ID NO: 1724) | PEgRNA spacer (GG; GGA PAM) |
| 1725 | TTGCTGGGTTTCCAAAGT (SEQ ID NO: 1725) | PEgRNA spacer (GG; GGA PAM) |
| 1726 | CTTGCTGGGTTTCCAAAGT (SEQ ID NO: 1726) | PEgRNA spacer (GG; GGA PAM) |
| 216 | TCTTGCTGGGTTTCCAAAGT (SEQ ID NO: 216) | PEgRNA spacer (GG; GGA PAM) |
| 1727 | GTCTTGCTGGGTTTCCAAAGT (SEQ ID NO: 1727) | PEgRNA spacer (GG; GGA PAM) |
| 1728 | TGTCTTGCTGGGTTTCCAAAGT (SEQ ID NO: 1728) | PEgRNA spacer (GG; GGA PAM) |
| 1729 | TTGGA | PBS |
| 1730 | TTGGAA | PBS |
| 1731 | TTGGAAA | PBS |
| 1732 | TTGGAAAC | PBS |
| 1733 | TTGGAAACC | PBS |
| 1734 | TTGGAAACCC (SEQ ID NO: 1734) | PBS |
| 1735 | TTGGAAACCCA (SEQ ID NO: 1735) | PBS |
| 1736 | TTGGAAACCCAG (SEQ ID NO: 1736) | PBS |
| 1737 | TTGGAAACCCAGC (SEQ ID NO: 1737) | PBS |
| 1738 | TTGGAAACCCAGCA (SEQ ID NO: 1738) | PBS |
| 1739 | TTGGAAACCCAGCAA (SEQ ID NO: 1739) | PBS |
| 1740 | TTGGAAACCCAGCAAG (SEQ ID NO: 1740) | PBS |
| 1741 | TTGGAAACCCAGCAAGA (SEQ ID NO: 1741) | PBS |
| 1742 | TTGGAAACCCAGCAAGAC (SEQ ID NO: 1742) | PBS |
| 1743 | TTGGAAACCCAGCAAGACA (SEQ ID NO: 1743) | PBS |
| 1744 | GATCAGGCTAGCACT (SEQ ID NO: 1744) | RTT*1, 2, 5, 6 (GG-to-GC; GGA-to-GCT PAM |
| | | silencing edit) |
| 1745 | GATCAGGCTAGTACT (SEQ ID NO: 1745) | RTT*1, 3, 5, 7 (GG-to-AC; GGA-to-ACT PAM |
| | | silencing edit) |
| 1746 | GATCAGGCTTCCACT (SEQ ID NO: 1746) | RTT*1, 4, 5, 8 |
| 1747 | GGATCAGGCTAGCACT (SEQ ID NO: 1747) | RTT*1, 2, 5, 6 (GG-to-GC; GGA-to-GCT PAM |
| | | silencing edit) |
| 1748 | GGATCAGGCTAGTACT (SEQ ID NO: 1748) | RTT*1, 3, 5, 7 (GG-to-AC; GGA-to-ACT PAM |
| | | silencing edit) |
| 1749 | GGATCAGGCTTCCACT (SEQ ID NO: 1749) | RTT*1, 4, 5, 8 |
| 1750 | GGGATCAGGCTAGCACT (SEQ ID NO: 1750) | RTT*1, 2, 5, 6 (GG-to-GC; GGA-to-GCT PAM |
| | | silencing edit) |
| 1751 | GGGATCAGGCTAGTACT (SEQ ID NO: 1751) | RTT*1, 3, 5, 7 (GG-to-AC; GGA-to-ACT PAM |
| | | silencing edit) |
| 1752 | GGGATCAGGCTTCCACT (SEQ ID NO: 1752) | RTT*1, 4, 5, 8 |
| 1753 | TGGGATCAGGCTAGCACT (SEQ ID NO: 1753) | RTT*1, 2, 5, 6 (GG-to-GC; GGA-to-GCT PAM |
| | | silencing edit) |
| 1754 | TGGGATCAGGCTAGTACT (SEQ ID NO: 1754) | RTT*1, 3, 5, 7 (GG-to-AC; GGA-to-ACT PAM |
| | | silencing edit) |
| 1755 | TGGGATCAGGCTTCCACT (SEQ ID NO: 1755) | RTT*1, 4, 5, 8 |
| 1756 | ATGGGATCAGGCTAGCACT (SEQ ID NO: 1756) | RTT*1, 2, 5, 6 (GG-to-GC; GGA-to-GCT PAM |
| | | silencing edit) |
| 1757 | ATGGGATCAGGCTAGTACT (SEQ ID NO: 1757) | RTT*1, 3, 5, 7 (GG-to-AC; GGA-to-ACT PAM |
| | | silencing edit) |
| 1758 | ATGGGATCAGGCTTCCACT (SEQ ID NO: 1758) | RTT*1, 4, 5, 8 |
| 1759 | TATGGGATCAGGCTAGCACT (SEQ ID NO: 1759) | RTT*1, 2, 5, 6 (GG-to-GC; GGA-to-GCT PAM |
| | | silencing edit) |
| 1760 | TATGGGATCAGGCTAGTACT (SEQ ID NO: 1760) | RTT*1, 3, 5, 7 (GG-to-AC; GGA-to-ACT PAM |
| | | silencing edit) |
| 132 | TATGGGATCAGGCTTCCACT (SEQ ID NO: 132) | RTT*1, 4, 5, 8 |
| 1761 | GTATGGGATCAGGCTAGCACT (SEQ ID NO: 1761) | RTT*1, 2, 5, 6 (GG-to-GC; GGA-to-GCT PAM |
| | | silencing edit) |
| 1762 | GTATGGGATCAGGCTAGTACT (SEQ ID NO: 1762) | RTT*1, 3, 5, 7 (GG-to-AC; GGA-to-ACT PAM |
| | | silencing edit) |
| 1763 | GTATGGGATCAGGCTTCCACT (SEQ ID NO: 1763) | RTT*1, 4, 5, 8 |
| 1764 | TGTATGGGATCAGGCTAGCACT (SEQ ID NO: | RTT*1, 2, 5, 6 (GG-to-GC; GGA-to-GCT PAM |
| | 1764) | silencing edit) |
| 1765 | TGTATGGGATCAGGCTAGTACT (SEQ ID NO: 1765) | RTT*1, 3, 5, 7 (GG-to-AC; GGA-to-ACT PAM |
| | | silencing edit) |
| 1766 | TGTATGGGATCAGGCTTCCACT (SEQ ID NO: 1766) | RTT*1, 4, 5, 8 |
| 1767 | CTGTATGGGATCAGGCTAGCACT (SEQ ID NO: | RTT*1, 2, 5, 6 (GG-to-GC; GGA-to-GCT PAM |
| | 1767) | silencing edit) |
| 1768 | CTGTATGGGATCAGGCTAGTACT (SEQ ID NO: | RTT*1, 3, 5, 7 (GG-to-AC; GGA-to-ACT PAM |
| | 1768) | silencing edit) |
| 1769 | CTGTATGGGATCAGGCTTCCACT (SEQ ID NO: | RTT*1, 4, 5, 8 |
| | 1769) | |
| 1770 | TCTGTATGGGATCAGGCTAGCACT (SEQ ID NO: | RTT*1, 2, 5, 6 (GG-to-GC; GGA-to-GCT PAM |
| | 1770) | silencing edit) |
| 1771 | TCTGTATGGGATCAGGCTAGTACT (SEQ ID NO: | RTT*1, 3, 5, 7 (GG-to-AC; GGA-to-ACT PAM |
| | 1771) | silencing edit) |
| 1772 | TCTGTATGGGATCAGGCTTCCACT (SEQ ID NO: | RTT*1, 4, 5, 8 |
| | 1772) | |
| 1773 | TTCTGTATGGGATCAGGCTAGCACT (SEQ ID NO: | RTT*1, 2, 5, 6 (GG-to-GC; GGA-to-GCT PAM |
| | 1773) | silencing edit) |
| 1774 | TTCTGTATGGGATCAGGCTAGTACT (SEQ ID NO: | RTT*1, 3, 5, 7 (GG-to-AC; GGA-to-ACT PAM |
| | 1774) | silencing edit) |
| 1775 | TTCTGTATGGGATCAGGCTTCCACT (SEQ ID NO: | RTT*1, 4, 5, 8 |
| | 1775) | |
| 1776 | TTTCTGTATGGGATCAGGCTAGCACT (SEQ ID NO: | RTT*1, 2, 5, 6 (GG-to-GC; GGA-to-GCT PAM |
| | 1776) | silencing edit) |
| 1777 | TTTCTGTATGGGATCAGGCTAGTACT (SEQ ID NO: | RTT*1, 3, 5, 7 (GG-to-AC; GGA-to-ACT PAM |
| | 1777) | silencing edit) |
| 1778 | TTTCTGTATGGGATCAGGCTTCCACT (SEQ ID NO: | RTT*1, 4, 5, 8 |
| | 1778) | |
| 1779 | CTTTCTGTATGGGATCAGGCTAGCACT (SEQ ID | RTT*1, 2, 5, 6 (GG-to-GC; GGA-to-GCT PAM |
| | NO: 1779) | silencing edit) |
| 1780 | CTTTCTGTATGGGATCAGGCTAGTACT (SEQ ID | RTT*1, 3, 5, 7 (GG-to-AC; GGA-to-ACT PAM |
| | NO: 1780) | silencing edit) |
| 1781 | CTTTCTGTATGGGATCAGGCTTCCACT (SEQ ID | RTT*1, 4, 5, 8 |
| | NO: 1781) | |
| 1782 | GCTTTCTGTATGGGATCAGGCTAGCACT (SEQ ID | RTT*1, 2, 5, 6 (GG-to-GC; GGA-to-GCT PAM |
| | NO: 1782) | silencing edit) |
| 1783 | GCTTTCTGTATGGGATCAGGCTAGTACT (SEQ ID | RTT*1, 3, 5, 7 (GG-to-AC; GGA-to-ACT PAM |
| | NO: 1783) | silencing edit) |
| 1784 | GCTTTCTGTATGGGATCAGGCTTCCACT (SEQ ID | RTT*1, 4, 5, 8 |
| | NO: 1784) | |
| 1785 | AGCTTTCTGTATGGGATCAGGCTAGCACT (SEQ | RTT*1, 2, 5, 6 (GG-to-GC; GGA-to-GCT PAM |
| | ID NO: 1785) | silencing edit) |
| 1786 | AGCTTTCTGTATGGGATCAGGCTAGTACT (SEQ | RTT*1, 3, 5, 7 (GG-to-AC; GGA-to-ACT PAM |
| | ID NO: 1786) | silencing edit) |
| 1787 | AGCTTTCTGTATGGGATCAGGCTTCCACT (SEQ ID | RTT*1, 4, 5, 8 |
| | NO: 1787) | |
| 1788 | AAGCTTTCTGTATGGGATCAGGCTAGCACT (SEQ | RTT*1, 2, 5, 6 (GG-to-GC; GGA-to-GCT PAM |
| | ID NO: 1788) | silencing edit) |
| 1789 | AAGCTTTCTGTATGGGATCAGGCTAGTACT (SEQ | RTT*1, 3, 5, 7 (GG-to-AC; GGA-to-ACT PAM |
| | ID NO: 1789) | silencing edit) |
| 1790 | AAGCTTTCTGTATGGGATCAGGCTTCCACT (SEQ | RTT*1, 4, 5, 8 |
| | ID NO: 1790) | |
| 1791 | GAAGCTTTCTGTATGGGATCAGGCTAGCACT | RTT*1, 2, 5, 6 (GG-to-GC; GGA-to-GCT PAM |
| | (SEQ ID NO: 1791) | silencing edit) |
| 1792 | GAAGCTTTCTGTATGGGATCAGGCTAGTACT | RTT*1, 3, 5, 7 (GG-to-AC; GGA-to-ACT PAM |
| | (SEQ ID NO: 1792) | silencing edit) |
| 1793 | GAAGCTTTCTGTATGGGATCAGGCTTCCACT | RTT*1, 4, 5, 8 |
| | (SEQ ID NO: 1793) | |
| 1794 | AGAAGCTTTCTGTATGGGATCAGGCTAGCACT | RTT*1, 2, 5, 6 (GG-to-GC; GGA-to-GCT PAM |
| | (SEQ ID NO: 1794) | silencing edit) |
| 1795 | AGAAGCTTTCTGTATGGGATCAGGCTAGTACT | RTT*1, 3, 5, 7 (GG-to-AC; GGA-to-ACT PAM |
| | (SEQ ID NO: 1795) | silencing edit) |
| 1796 | AGAAGCTTTCTGTATGGGATCAGGCTTCCACT | RTT*1, 4, 5, 8 |
| | (SEQ ID NO: 1796) | |
| 1797 | CAGAAGCTTTCTGTATGGGATCAGGCTAGCACT | RTT*1, 2, 5, 6 (GG-to-GC; GGA-to-GCT PAM |
| | (SEQ ID NO: 1797) | silencing edit) |
| 1798 | CAGAAGCTTTCTGTATGGGATCAGGCTAGTACT | RTT*1, 3, 5, 7 (GG-to-AC; GGA-to-ACT PAM |
| | (SEQ ID NO: 1798) | silencing edit) |
| 1799 | CAGAAGCTTTCTGTATGGGATCAGGCTTCCACT | RTT*1, 4, 5, 8 |
| | (SEQ ID NO: 1799) | |
| 1800 | GCAGAAGCTTTCTGTATGGGATCAGGCTAGCACT | RTT*1, 2, 5, 6 (GG-to-GC; GGA-to-GCT PAM |
| | (SEQ ID NO: 1800) | silencing edit) |
| 1801 | GCAGAAGCTTTCTGTATGGGATCAGGCTAGTACT | RTT*1, 3, 5, 7 (GG-to-AC; GGA-to-ACT PAM |
| | (SEQ ID NO: 1801) | silencing edit) |
| 1802 | GCAGAAGCTTTCTGTATGGGATCAGGCTTCCACT | RTT*1, 4, 5, 8 |
| | (SEQ ID NO: 1802) | |
| 1803 | TGCAGAAGCTTTCTGTATGGGATCAGGCTAGCAC | RTT*1, 2, 5, 6 (GG-to-GC; GGA-to-GCT PAM |
| | T (SEQ ID NO: 1803) | silencing edit) |
| 1804 | TGCAGAAGCTTTCTGTATGGGATCAGGCTAGTAC | RTT*1, 3, 5, 7 (GG-to-AC; GGA-to-ACT PAM |
| | T (SEQ ID NO: 1804) | silencing edit) |
| 1805 | TGCAGAAGCTTTCTGTATGGGATCAGGCTTCCAC | RTT*1, 4, 5, 8 |
| | T (SEQ ID NO: 1805) | |
| 1806 | ATGCAGAAGCTTTCTGTATGGGATCAGGCTAGCA | RTT*1, 2, 5, 6 (GG-to-GC; GGA-to-GCT PAM |
| | CT (SEQ ID NO: 1806) | silencing edit) |
| 1807 | ATGCAGAAGCTTTCTGTATGGGATCAGGCTAGTA | RTT*1, 3, 5, 7 (GG-to-AC; GGA-to-ACT PAM |
| | CT (SEQ ID NO: 1807) | silencing edit) |
| 1808 | ATGCAGAAGCTTTCTGTATGGGATCAGGCTTCCA | RTT*1, 4, 5, 8 |
| | CT (SEQ ID NO: 1808) | |
| 1809 | GATGCAGAAGCTTTCTGTATGGGATCAGGCTAGC | RTT*1, 2, 5, 6 (GG-to-GC; GGA-to-GCT PAM |
| | ACT (SEQ ID NO: 1809) | silencing edit) |
| 1810 | GATGCAGAAGCTTTCTGTATGGGATCAGGCTAGT | RTT*1, 3, 5, 7 (GG-to-AC; GGA-to-ACT PAM |
| | ACT (SEQ ID NO: 1810) | silencing edit) |
| 1811 | GATGCAGAAGCTTTCTGTATGGGATCAGGCTTCC | RTT*1, 4, 5, 8 |
| | ACT (SEQ ID NO: 1811) | |
| 1812 | GGATGCAGAAGCTTTCTGTATGGGATCAGGCTAG | RTT*1, 2, 5, 6 (GG-to-GC; GGA-to-GCT PAM |
| | CACT (SEQ ID NO: 1812) | silencing edit) |
| 1813 | GGATGCAGAAGCTTTCTGTATGGGATCAGGCTAG | RTT*1, 3, 5, 7 (GG-to-AC; GGA-to-ACT PAM |
| | TACT (SEQ ID NO: 1813) | silencing edit) |
| 1814 | GGATGCAGAAGCTTTCTGTATGGGATCAGGCTTC | RTT*1, 4, 5, 8 |
| | CACT (SEQ ID NO: 1814) | |
| 1815 | TGGATGCAGAAGCTTTCTGTATGGGATCAGGCTA | RTT*1, 2, 5, 6 (GG-to-GC; GGA-to-GCT PAM |
| | GCACT (SEQ ID NO: 1815) | silencing edit) |
| 1816 | TGGATGCAGAAGCTTTCTGTATGGGATCAGGCTA | RTT*1, 3, 5, 7 (GG-to-AC; GGA-to-ACT PAM |
| | GTACT (SEQ ID NO: 1816) | silencing edit) |
| 1817 | TGGATGCAGAAGCTTTCTGTATGGGATCAGGCTT | RTT*1, 4, 5, 8 |
| | CCACT (SEQ ID NO: 1817) | |
| 1818 | TTGGATGCAGAAGCTTTCTGTATGGGATCAGGCT | RTT*1, 2, 5, 6 (GG-to-GC; GGA-to-GCT PAM |
| | AGCACT (SEQ ID NO: 1818) | silencing edit) |
| 1819 | TTGGATGCAGAAGCTTTCTGTATGGGATCAGGCT | RTT*1, 3, 5, 7 (GG-to-AC; GGA-to-ACT PAM |
| | AGTACT (SEQ ID NO: 1819) | silencing edit) |
| 1820 | TTGGATGCAGAAGCTTTCTGTATGGGATCAGGCT | RTT*1, 4, 5, 8 |
| | TCCACT (SEQ ID NO: 1820) | |
| 24 | AAGATGGCTCAAGATTCAGT (SEQ ID NO: 24) | PE3 ngRNA spacer (AG;AGA PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b*1 ngRNA spacer (GG PAM) |
| 27 | AGATCTTTCTTGTGATTATC (SEQ ID NO: 27) | PE3 ngRNA spacer (AG PAM) |
| 1274 | ATGGGATCAGGCTAGCACTT (SEQ ID NO: 1274) | PE3b*2 ngRNA spacer (TG PAM) |
| 1275 | ATGGGATCAGGCTAGTACTT (SEQ ID NO: 1275) | PE3b*3 ngRNA spacer (TG PAM) |
| 28 | ATGGGATCAGGCTTCCACTT (SEQ ID NO: 28) | PE3b*4 ngRNA spacer (TG PAM) |
| 1108 | CAGTGCTGGACAGGCTGCTG (SEQ ID NO: 1108) | PE3 ngRNA spacer (TG;TGA PAM) |
| 29 | CCTTTTCGCTTTTTCCAAGA (SEQ ID NO: 29) | PE3 ngRNA spacer (TG PAM) |
| 1110 | CTGGACAGGCTGCTGTGAAG (SEQ ID NO: 1110) | PE3 ngRNA spacer (GG;GGA PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b*5 ngRNA spacer (AG PAM) |
| 32 | GATGCAGAAGCTTTCTGTAT (SEQ ID NO: 32) | PE3 ngRNA spacer (GG;GGA PAM) |
| 33 | GATTCAGTAGATCTTTCTTG (SEQ ID NO: 33) | PE3 ngRNA spacer (TG;TGA PAM) |
| 1111 | GCTGGACAGGCTGCTGTGAA (SEQ ID NO: 1111) | PE3 ngRNA spacer (GG PAM) |
| 34 | GCTTTTTCCAAGATGGCTCA (SEQ ID NO: 34) | PE3 ngRNA spacer (AG;AGA PAM) |
| 35 | GGATGCAGAAGCTTTCTGTA (SEQ ID NO: 35) | PE3 ngRNA spacer (TG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG;AGA PAM) |
| 39 | TCCAAGATGGCTCAAGATTC (SEQ ID NO: 39) | PE3 ngRNA spacer (AG PAM) |
| 1112 | TCCTTCAGTGCTGGACAGGC (SEQ ID NO: 1112) | PE3 ngRNA spacer (TG PAM) |
| 41 | TGATTATCAGTTTTGGATGC (SEQ ID NO: 41) | PE3 ngRNA spacer (AG;AGA PAM) |
| 1113 | TGCTGGACAGGCTGCTGTGA (SEQ ID NO: 1113) | PE3 ngRNA spacer (AG PAM) |
| 1821 | TGGGATCAGGCTAGCACTTT (SEQ ID NO: 1821) | PE3b*6 ngRNA spacer (GG;GGA PAM) |
| 1399 | TGGGATCAGGCTAGTACTTT (SEQ ID NO: 1399) | PE3b*7 ngRNA spacer (GG;GGA PAM) |
| 43 | TGGGATCAGGCTTCCACTTT (SEQ ID NO: 43) | PE3b*8 ngRNA spacer (GG;GGA PAM) |
| 44 | TTATCAGTTTTGGATGCAGA (SEQ ID NO: 44) | PE3 ngRNA spacer (AG PAM) |
| 1114 | TTCAGTGCTGGACAGGCTGC (SEQ ID NO: 1114) | PE3 ngRNA spacer (TG PAM) |
| 45 | TTCTTGTGATTATCAGTTTT (SEQ ID NO: 45) | PE3 ngRNA spacer (GG;GGA PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b*5 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b*4 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG PAM), contains 3′ TTTT |
| | AATAGCAAGTTAAAATAAGGCTAGTCCGTTATCA | |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b*5 ngRNA (AG PAM), contains 3′ TTTT |
| | AATAGCAAGTTAAAATAAGGCTAGTCCGTTATCA | |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains 3′ TTTT |
| | AATAGCAAGTTAAAATAAGGCTAGTCCGTTATCA | |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b*4 ngRNA (TG PAM), contains 3′ TTTT |
| | AAATAGCAAGTTAAAATAAGGCTAGTCCGTTATC | |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b*5 ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; 3′ TTTT |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b*4 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; 3′ TTTT |
| | TTGACGCGTCTCTACGTGGGGGCGCGTTTT (SEQ | |
| | ID NO: 60) | |

TABLE 24

| Sequence | | |
|---|---|---|
| Number | Sequence | Description |
| 1822 | AGACAGGTGTCTTGCTG (SEQ ID NO: 1822) | PEgRNA spacer (GG PAM) |
| 1823 | AAGACAGGTGTCTTGCTG (SEQ ID NO: 1823) | PEgRNA spacer (GG PAM) |
| 1824 | GAAGACAGGTGTCTTGCTG (SEQ ID NO: 1824) | PEgRNA spacer (GG PAM) |
| 217 | TGAAGACAGGTGTCTTGCTG (SEQ ID NO: 217) | PEgRNA spacer (GG PAM) |
| 1825 | GTGAAGACAGGTGTCTTGCTG (SEQ ID NO: 1825) | PEgRNA spacer (GG PAM) |
| 1826 | CGTGAAGACAGGTGTCTTGCTG (SEQ ID NO: | PEgRNA spacer (GG PAM) |
| | 1826) | |
| 1827 | CAAGA | PBS |
| 1828 | CAAGAC | PBS |
| 1829 | CAAGACA | PBS |
| 1830 | CAAGACAC | PBS |
| 1831 | CAAGACACC | PBS |
| 1832 | CAAGACACCT (SEQ ID NO: 1832) | PBS |
| 1833 | CAAGACACCTG (SEQ ID NO: 1833) | PBS |
| 1834 | CAAGACACCTGT (SEQ ID NO: 1834) | PBS |
| 1835 | CAAGACACCTGTC (SEQ ID NO: 1835) | PBS |
| 1836 | CAAGACACCTGTCT (SEQ ID NO: 1836) | PBS |
| 1837 | CAAGACACCTGTCTT (SEQ ID NO: 1837) | PBS |
| 1838 | CAAGACACCTGTCTTC (SEQ ID NO: 1838) | PBS |
| 1839 | CAAGACACCTGTCTTCA (SEQ ID NO: 1839) | PBS |
| 1840 | CAAGACACCTGTCTTCAC (SEQ ID NO: 1840) | PBS |
| 1841 | CAAGACACCTGTCTTCACG (SEQ ID NO: 1841) | PBS |
| 1842 | GATCAGGCTTCCACTTTGGAAACCCAG (SEQ ID | RTT |
| | NO: 1842) | |
| 1843 | GGATCAGGCTTCCACTTTGGAAACCCAG (SEQ ID | RTT |
| | NO: 1843) | |
| 1844 | GGGATCAGGCTTCCACTTTGGAAACCCAG (SEQ | RTT |
| | ID NO: 1844) | |
| 1845 | TGGGATCAGGCTTCCACTTTGGAAACCCAG (SEQ | RTT |
| | ID NO: 1845) | |
| 1846 | ATGGGATCAGGCTTCCACTTTGGAAACCCAG | RTT |
| | (SEQ ID NO: 1846) | |
| 1847 | TATGGGATCAGGCTTCCACTTTGGAAACCCAG | RTT |
| | (SEQ ID NO: 1847) | |
| 1848 | GTATGGGATCAGGCTTCCACTTTGGAAACCCAG | RTT |
| | (SEQ ID NO: 1848) | |
| 1849 | TGTATGGGATCAGGCTTCCACTTTGGAAACCCAG | RTT |
| | (SEQ ID NO: 1849) | |
| 1850 | CTGTATGGGATCAGGCTTCCACTTTGGAAACCCA | RTT |
| | G (SEQ ID NO: 1850) | |
| 1851 | TCTGTATGGGATCAGGCTTCCACTTTGGAAACCC | RTT |
| | AG (SEQ ID NO: 1851) | |
| 1852 | TTCTGTATGGGATCAGGCTTCCACTTTGGAAACC | RTT |
| | CAG (SEQ ID NO: 1852) | |
| 1853 | TTTCTGTATGGGATCAGGCTTCCACTTTGGAAAC | RT |
| | CCAG (SEQ ID NO: 1853) | |
| 1854 | CTTTCTGTATGGGATCAGGCTTCCACTTTGGAAA | RTT |
| | CCCAG (SEQ ID NO: 1854) | |
| 1855 | GCTTTCTGTATGGGATCAGGCTTCCACTTTGGAA | RTT |
| | ACCCAG (SEQ ID NO: 1855) | |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b ngRNA spacer (GG PAM) |
| 27 | AGATCTTTCTTGTGATTATC (SEQ ID NO: 27) | PE3 ngRNA spacer (AG PAM) |
| 28 | ATGGGATCAGGCTTCCACTT (SEQ ID NO: 28) | PE3b ngRNA spacer (TG PAM) |
| 29 | CCTTTTCGCTTTTTCCAAGA (SEQ ID NO: 29) | PE3 ngRNA spacer (TG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b ngRNA spacer (AG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG PAM) |
| 36 | GGCTTCCACTTTGGAAACCC (SEQ ID NO: 36) | PE3 ngRNA spacer (AG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 40 | TCCACTTTGGAAACCCAGCA (SEQ ID NO: 40) | PE3 ngRNA spacer (AG PAM) |
| 41 | TGATTATCAGTTTTGGATGC (SEQ ID NO: 41) | PE3 ngRNA spacer (AG PAM) |
| 43 | TGGGATCAGGCTTCCACTTT (SEQ ID NO: 43) | PE3b ngRNA spacer (GG PAM) |
| 44 | TTATCAGTTTTGGATGCAGA (SEQ ID NO: 44) | PE3 ngRNA spacer (AG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 52 | ATGGGATCAGGCTTCCACTTGTTTTAGAGCTAGA | PE3b ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 52) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 56 | GATGGGATCAGGCTTCCACTTGTTTTAGAGCTAG | PE3b ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 56) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |
| 60 | GATGGGATCAGGCTTCCACTTGTTTTAGAGCTAG | PE3b ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 60) | |

TABLE 25

| Sequence | | |
|---|---|---|
| Number | Sequence | Description |
| 1856 | TTATCAGTTTTGGATGC (SEQ ID NO: 1856) | PEgRNA spacer (AG; AGA PAM) |
| 1857 | ATTATCAGTTTTGGATGC (SEQ ID NO: 1857) | PEgRNA spacer (AG; AGA PAM) |
| 1858 | GATTATCAGTTTTGGATGC (SEQ ID NO: 1858) | PEgRNA spacer (AG; AGA PAM) |
| 41 | TGATTATCAGTTTTGGATGC (SEQ ID NO: 41) | PEgRNA spacer (AG; AGA PAM) |
| 1859 | GTGATTATCAGTTTTGGATGC (SEQ ID NO: 1859) | PEgRNA spacer (AG; AGA PAM) |
| 1860 | TGTGATTATCAGTTTTGGATGC (SEQ ID NO: 1860) | PEgRNA spacer (AG; AGA PAM) |
| 1861 | TCCAA | PBS |
| 1862 | TCCAAA | PBS |
| 1863 | TCCAAAA | PBS |
| 1864 | TCCAAAAC | PBS |
| 1865 | TCCAAAACT | PBS |
| 1866 | TCCAAAACTG (SEQ ID NO: 1866) | PBS |
| 1867 | TCCAAAACTGA (SEQ ID NO: 1867) | PBS |
| 1868 | TCCAAAACTGAT (SEQ ID NO: 1868) | PBS |
| 1869 | TCCAAAACTGATA (SEQ ID NO: 1869) | PBS |
| 1870 | TCCAAAACTGATAA (SEQ ID NO: 1870) | PBS |
| 1871 | TCCAAAACTGATAAT (SEQ ID NO: 1871) | PBS |
| 1872 | TCCAAAACTGATAATC (SEQ ID NO: 1872) | PBS |
| 1873 | TCCAAAACTGATAATCA (SEQ ID NO: 1873) | PBS |
| 1874 | TCCAAAACTGATAATCAC (SEQ ID NO: 1874) | PBS |
| 1875 | TCCAAAACTGATAATCACA (SEQ ID NO: 1875) | PBS |
| 1876 | CCCATACAGAAAGCTTCTGCA (SEQ ID NO: 1876) | RTT*1, 2, 4 |
| 1877 | CCCATACAGAAAGCTTTTGCA (SEQ ID NO: 1877) | RTT*1, 3, 4 (AG-to-AA; AGA-to-AAA PAM |
| | | silencing edit) |
| 1878 | TCCCATACAGAAAGCTTCTGCA (SEQ ID NO: 1878) | RTT*1, 2, 4 |
| 1879 | TCCCATACAGAAAGCTTTTGCA (SEQ ID NO: 1879) | RTT*1, 3, 4 (AG-to-AA; AGA-to-AAA PAM |
| | | silencing edit) |
| 1880 | ATCCCATACAGAAAGCTTCTGCA (SEQ ID NO: | RTT*1, 2, 4 |
| | 1880) | |
| 1881 | ATCCCATACAGAAAGCTTTTGCA (SEQ ID NO: | RTT*1, 3, 4 (AG-to-AA; AGA-to-AAA PAM |
| | 1881) | silencing edit) |
| 1882 | GATCCCATACAGAAAGCTTCTGCA (SEQ ID NO: | RTT*1, 2, 4 |
| | 1882) | |
| 1883 | GATCCCATACAGAAAGCTTTTGCA (SEQ ID NO: | RTT*1, 3, 4 (AG-to-AA; AGA-to-AAA PAM |
| | 1883) | silencing edit) |
| 1884 | TGATCCCATACAGAAAGCTTCTGCA (SEQ ID NO: | RTT*1, 2, 4 |
| | 1884) | |
| 1885 | TGATCCCATACAGAAAGCTTTTGCA (SEQ ID NO: | RTT*1, 3, 4 (AG-to-AA; AGA-to-AAA PAM |
| | 1885) | silencing edit) |
| 1886 | CTGATCCCATACAGAAAGCTTCTGCA (SEQ ID | RTT*1, 2, 4 |
| | NO: 1886) | |
| 1887 | CTGATCCCATACAGAAAGCTTTTGCA (SEQ ID | RTT*1, 3, 4 (AG-to-AA; AGA-to-AAA PAM |
| | NO: 1887) | silencing edit) |
| 1888 | CCTGATCCCATACAGAAAGCTTCTGCA (SEQ ID | RTT*1, 2, 4 |
| | NO: 1888) | |
| 1889 | CCTGATCCCATACAGAAAGCTTTTGCA (SEQ ID | RTT*1, 3, 4 (AG-to-AA; AGA-to-AAA PAM |
| | NO: 1889) | silencing edit) |
| 1890 | GCCTGATCCCATACAGAAAGCTTCTGCA (SEQ ID | RTT*1, 2, 4 |
| | NO: 1890) | |
| 1891 | GCCTGATCCCATACAGAAAGCTTTTGCA (SEQ ID | RTT*1, 3, 4 (AG-to-AA; AGA-to-AAA PAM |
| | NO: 1891) | silencing edit) |
| 1892 | AGCCTGATCCCATACAGAAAGCTTCTGCA (SEQ | RTT*1, 2, 4 |
| | ID NO: 1892) | |
| 1893 | AGCCTGATCCCATACAGAAAGCTTTTGCA (SEQ | RTT*1, 3, 4 (AG-to-AA; AGA-to-AAA PAM |
| | ID NO: 1893) | silencing edit) |
| 1894 | AAGCCTGATCCCATACAGAAAGCTTCTGCA (SEQ | RTT*1, 2, 4 |
| | ID NO: 1894) | |
| 1895 | AAGCCTGATCCCATACAGAAAGCTTTTGCA (SEQ | RTT*1, 3, 4 (AG-to-AA; AGA-to-AAA PAM |
| | ID NO: 1895) | silencing edit) |
| 1896 | GAAGCCTGATCCCATACAGAAAGCTTCTGCA | RTT*1, 2, 4 |
| | (SEQ ID NO: 1896) | |
| 1897 | GAAGCCTGATCCCATACAGAAAGCTTTTGCA | RTT*1, 3, 4 (AG-to-AA; AGA-to-AAA PAM |
| | (SEQ ID NO: 1897) | silencing edit) |
| 1898 | GGAAGCCTGATCCCATACAGAAAGCTTCTGCA | RTT*1, 2, 4 |
| | (SEQ ID NO: 1898) | |
| 1899 | GGAAGCCTGATCCCATACAGAAAGCTTTTGCA | RTT*1, 3, 4 (AG-to-AA; AGA-to-AAA PAM |
| | (SEQ ID NO: 1899) | silencing edit) |
| 1900 | TGGAAGCCTGATCCCATACAGAAAGCTTCTGCA | RTT*1, 2, 4 |
| | (SEQ ID NO: 1900) | |
| 1901 | TGGAAGCCTGATCCCATACAGAAAGCTTTTGCA | RTT*1, 3, 4 (AG-to-AA; AGA-to-AAA PAM |
| | (SEQ ID NO: 1901) | silencing edit) |
| 1902 | GTGGAAGCCTGATCCCATACAGAAAGCTTCTGC | RTT*1, 2, 4 |
| | A (SEQ ID NO: 1902) | |
| 1903 | GTGGAAGCCTGATCCCATACAGAAAGCTTTTGCA | RTT*1, 3, 4 (AG-to-AA; AGA-to-AAA PAM |
| | (SEQ ID NO: 1903) | silencing edit) |
| 1904 | AGTGGAAGCCTGATCCCATACAGAAAGCTTCTG | RTT*1, 2, 4 |
| | CA (SEQ ID NO: 1904) | |
| 1905 | AGTGGAAGCCTGATCCCATACAGAAAGCTTTTGC | RTT*1, 3, 4 (AG-to-AA; AGA-to-AAA PAM |
| | A (SEQ ID NO: 1905) | silencing edit) |
| 1906 | AAGTGGAAGCCTGATCCCATACAGAAAGCTTCT | RTT*1, 2, 4 |
| | GCA (SEQ ID NO: 1906) | |
| 1907 | AAGTGGAAGCCTGATCCCATACAGAAAGCTTTTG | RTT*1, 3, 4 (AG-to-AA; AGA-to-AAA PAM |
| | CA (SEQ ID NO: 1907) | silencing edit) |
| 1908 | AAAGTGGAAGCCTGATCCCATACAGAAAGCTTC | RTT*1, 2, 4 |
| | TGCA (SEQ ID NO: 1908) | |
| 1909 | AAAGTGGAAGCCTGATCCCATACAGAAAGCTTTT | RTT*1, 3, 4 (AG-to-AA; AGA-to-AAA PAM |
| | GCA (SEQ ID NO: 1909) | silencing edit) |
| 1910 | CAAAGTGGAAGCCTGATCCCATACAGAAAGCTT | RTT*1, 2, 4 |
| | CTGCA (SEQ ID NO: 1910) | |
| 1911 | CAAAGTGGAAGCCTGATCCCATACAGAAAGCTT | RTT*1, 3, 4 (AG-to-AA; AGA-to-AAA PAM |
| | TTGCA (SEQ ID NO: 1911) | silencing edit) |
| 1912 | CCAAAGTGGAAGCCTGATCCCATACAGAAAGCT | RTT*1, 2, 4 |
| | TCTGCA (SEQ ID NO: 1912) | |
| 1913 | CCAAAGTGGAAGCCTGATCCCATACAGAAAGCT | RTT*1, 3, 4 (AG-to-AA; AGA-to-AAA PAM |
| | TTTGCA (SEQ ID NO: 1913) | silencing edit) |
| 1914 | TCCAAAGTGGAAGCCTGATCCCATACAGAAAGC | RTT*1, 2, 4 |
| | TTCTGCA (SEQ ID NO: 1914) | |
| 1915 | TCCAAAGTGGAAGCCTGATCCCATACAGAAAGC | RTT*1, 3, 4 (AG-to-AA; AGA-to-AAA PAM |
| | TTTTGCA (SEQ ID NO: 1915) | silencing edit) |
| 4 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b*1 ngRNA spacer (AG PAM) |
| 195 | ACATCTTCCTTAGGAACTCC (SEQ ID NO: 195) | PE3 ngRNA spacer (TG PAM) |
| 196 | ACTACTTACCATCTCTTTCA (SEQ ID NO: 196) | PE3 ngRNA spacer (AG PAM) |
| 197 | ACTCCTGGAACTGAGCCACG (SEQ ID NO: 197) | PE3 ngRNA spacer (TG; TGA PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG; GGA PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG; AGA PAM) |
| 203 | CGTGAAGACAGGTGTCTTGC (SEQ ID NO: 203) | PE3 ngRNA spacer (TG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 383 | GATCCCATACAGAAAGCTTC (SEQ ID NO: 383) | PE3b*2 ngRNA spacer (TG PAM) |
| 1916 | GATCCCATACAGAAAGCTTT (SEQ ID NO: 1916) | PE3b*3 ngRNA spacer (TG PAM) |
| 210 | GGCTTCATACATCTTCCTTA (SEQ ID NO: 210) | PE3 ngRNA spacer (GG; GGA PAM) |
| 211 | GGGTTTCCAAAGTGGAAGCC (SEQ ID NO: 211) | PE3 ngRNA spacer (TG; TGA PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b*4 ngRNA spacer (AG; AGA PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG; TGA PAM) |
| 216 | TCTTGCTGGGTTTCCAAAGT (SEQ ID NO: 216) | PE3 ngRNA spacer (GG; GGA PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 26

| Sequence | | |
|---|---|---|
| Number | Sequence | Description |
| 1917 | TGGGTTTCCAAAGTGGA (SEQ ID NO: 1917) | PEgRNA spacer (AG PAM) |
| 1918 | CTGGGTTTCCAAAGTGGA (SEQ ID NO: 1918) | PEgRNA spacer (AG PAM) |
| 1919 | GCTGGGTTTCCAAAGTGGA (SEQ ID NO: 1919) | PEgRNA spacer (AG PAM) |
| 218 | TGCTGGGTTTCCAAAGTGGA (SEQ ID NO: 218) | PEgRNA spacer (AG PAM) |
| 1920 | TTGCTGGGTTTCCAAAGTGGA (SEQ ID NO: 1920) | PEgRNA spacer (AG PAM) |
| 1921 | CTTGCTGGGTTTCCAAAGTGGA (SEQ ID NO: 1921) | PERNA spacer (AG PAM) |
| 1922 | ACTTT | PBS |
| 1923 | ACTTTG | PBS |
| 1924 | ACTTTGG | PBS |
| 1925 | ACTTTGGA | PBS |
| 1926 | ACTTTGGAA | PBS |
| 1927 | ACTTTGGAAA (SEQ ID NO: 1927) | PBS |
| 1928 | ACTTTGGAAAC (SEQ ID NO: 1928) | PBS |
| 1929 | ACTTTGGAAACC (SEQ ID NO: 1929) | PBS |
| 1930 | ACTTTGGAAACCC (SEQ ID NO: 1930) | PBS |
| 1931 | ACTTTGGAAACCCA (SEQ ID NO: 1931) | PBS |
| 1932 | ACTTTGGAAACCCAG (SEQ ID NO: 1932) | PBS |
| 1933 | ACTTTGGAAACCCAGC (SEQ ID NO: 1933) | PBS |
| 1934 | ACTTTGGAAACCCAGCA (SEQ ID NO: 1934) | PBS |
| 1935 | ACTTTGGAAACCCAGCAA (SEQ ID NO: 1935) | PBS |
| 1936 | ACTTTGGAAACCCAGCAAG (SEQ ID NO: 1936) | PBS |
| 1937 | GATCAGGCTTCC (SEQ ID NO: 1937) | RTT |
| 1938 | GGATCAGGCTTCC (SEQ ID NO: 1938) | RTT |
| 1939 | GGGATCAGGCTTCC (SEQ ID NO: 1939) | RTT |
| 1940 | TGGGATCAGGCTTCC (SEQ ID NO: 1940) | RTT |
| 1941 | ATGGGATCAGGCTTCC (SEQ ID NO: 1941) | RTT |
| 1942 | TATGGGATCAGGCTTCC (SEQ ID NO: 1942) | RTT |
| 1943 | GTATGGGATCAGGCTTCC (SEQ ID NO: 1943) | RTT |
| 1944 | TGTATGGGATCAGGCTTCC (SEQ ID NO: 1944) | RTT |
| 1945 | CTGTATGGGATCAGGCTTCC (SEQ ID NO: 1945) | RTT |
| 1946 | TCTGTATGGGATCAGGCTTCC (SEQ ID NO: 1946) | RTT |
| 1947 | TTCTGTATGGGATCAGGCTTCC (SEQ ID NO: 1947) | RTT |
| 1948 | TTTCTGTATGGGATCAGGCTTCC (SEQ ID NO: | RTT |
| | 1948) | |
| 1949 | CTTTCTGTATGGGATCAGGCTTCC (SEQ ID NO: | RTT |
| | 1949) | |
| 1950 | GCTTTCTGTATGGGATCAGGCTTCC (SEQ ID NO: | RTT |
| | 1950) | |
| 1951 | AGCTTTCTGTATGGGATCAGGCTTCC (SEQ ID NO: | RTT |
| | 1951) | |
| 1952 | AAGCTTTCTGTATGGGATCAGGCTTCC (SEQ ID | RTT |
| | NO: 1952) | |
| 1953 | GAAGCTTTCTGTATGGGATCAGGCTTCC (SEQ ID | RTT |
| | NO: 1953) | |
| 1954 | AGAAGCTTTCTGTATGGGATCAGGCTTCC (SEQ | RTT |
| | ID NO: 1954) | |
| 1955 | CAGAAGCTTTCTGTATGGGATCAGGCTTCC (SEQ | RTT |
| | ID NO: 1955) | |
| 1956 | GCAGAAGCTTTCTGTATGGGATCAGGCTTCC | RTT |
| | (SEQ ID NO: 1956) | |
| 1957 | TGCAGAAGCTTTCTGTATGGGATCAGGCTTCC | RTT |
| | (SEQ ID NO: 1957) | |
| 1958 | ATGCAGAAGCTTTCTGTATGGGATCAGGCTTCC | RTT |
| | (SEQ ID NO: 1958) | |
| 1959 | GATGCAGAAGCTTTCTGTATGGGATCAGGCTTCC | RTT |
| | (SEQ ID NO: 1959) | |
| 1960 | GGATGCAGAAGCTTTCTGTATGGGATCAGGCTTC | RTT |
| | C (SEQ ID NO: 1960) | |
| 1961 | TGGATGCAGAAGCTTTCTGTATGGGATCAGGCTT | RTT |
| | CC (SEQ ID NO: 1961) | |
| 1962 | TTGGATGCAGAAGCTTTCTGTATGGGATCAGGCT | RTT |
| | TCC (SEQ ID NO: 1962) | |
| 1963 | TTTGGATGCAGAAGCTTTCTGTATGGGATCAGGC | RTT |
| | TTCC (SEQ ID NO: 1963) | |
| 1964 | TTTTGGATGCAGAAGCTTTCTGTATGGGATCAGG | RTT |
| | CTTCC (SEQ ID NO: 1964) | |
| 1965 | GTTTTGGATGCAGAAGCTTTCTGTATGGGATCAG | RTT |
| | GCTTCC (SEQ ID NO: 1965) | |
| 24 | AAGATGGCTCAAGATTCAGT (SEQ ID NO: 24) | PE3 ngRNA spacer (AG PAM) |
| 25 | AAGATTCAGTAGATCTTTCT (SEQ ID NO: 25) | PE3 ngRNA spacer (TG PAM) |
| 26 | AAGCTTTCTGTATGGGATCA (SEQ ID NO: 26) | PE3b ngRNA spacer (GG PAM) |
| 27 | AGATCTTTCTTGTGATTATC (SEQ ID NO: 27) | PE3 ngRNA spacer (AG PAM) |
| 1107 | ATTTCCTTCAGTGCTGGACA (SEQ ID NO: 1107) | PE3 ngRNA spacer (GG PAM) |
| 1108 | CAGTGCTGGACAGGCTGCTG (SEQ ID NO: 1108) | PE3 ngRNA spacer (TG PAM) |
| 29 | CCTTTTCGCTTTTTCCAAGA (SEQ ID NO: 29) | PE3 ngRNA spacer (TG PAM) |
| 1110 | CTGGACAGGCTGCTGTGAAG (SEQ ID NO: 1110) | PE3 ngRNA spacer (GG PAM) |
| 30 | CTTTTCGCTTTTTCCAAGAT (SEQ ID NO: 30) | PE3 ngRNA spacer (GG PAM) |
| 31 | GAAGCTTTCTGTATGGGATC (SEQ ID NO: 31) | PE3b ngRNA spacer (AG PAM) |
| 32 | GATGCAGAAGCTTTCTGTAT (SEQ ID NO: 32) | PE3 ngRNA spacer (GG PAM) |
| 33 | GATTCAGTAGATCTTTCTTG (SEQ ID NO: 33) | PE3 ngRNA spacer (TG PAM) |
| 1111 | GCTGGACAGGCTGCTGTGAA (SEQ ID NO: 1111) | PE3 ngRNA spacer (GG PAM) |
| 34 | GCTTTTTCCAAGATGGCTCA (SEQ ID NO: 34) | PE3 ngRNA spacer (AG PAM) |
| 35 | GGATGCAGAAGCTTTCTGTA (SEQ ID NO: 35) | PE3 ngRNA spacer (TG PAM) |
| 37 | GTGAAGGGACATCACCTTTT (SEQ ID NO: 37) | PE3 ngRNA spacer (CG PAM) |
| 38 | TCACCTTTTCGCTTTTTCCA (SEQ ID NO: 38) | PE3 ngRNA spacer (AG PAM) |
| 39 | TCCAAGATGGCTCAAGATTC (SEQ ID NO: 39) | PE3 ngRNA spacer (AG PAM) |
| 1112 | TCCTTCAGTGCTGGACAGGC (SEQ ID NO: 1112) | PE3 ngRNA spacer (TG PAM) |
| 41 | TGATTATCAGTTTTGGATGC (SEQ ID NO: 41) | PE3 ngRNA spacer (AG PAM) |
| 1113 | TGCTGGACAGGCTGCTGTGA (SEQ ID NO: 1113) | PE3 ngRNA spacer (AG PAM) |
| 44 | TTATCAGTTTTGGATGCAGA (SEQ ID NO: 44) | PE3 ngRNA spacer (AG PAM) |
| 1114 | TTCAGTGCTGGACAGGCTGC (SEQ ID NO: 1114) | PE3 ngRNA spacer (TG PAM) |
| 45 | TTCTTGTGATTATCAGTTTT (SEQ ID NO: 45) | PE3 ngRNA spacer (GG PAM) |
| 46 | TTGTGATTATCAGTTTTGGA (SEQ ID NO: 46) | PE3 ngRNA spacer (TG PAM) |
| 47 | TTTCTTGTGATTATCAGTTT (SEQ ID NO: 47) | PE3 ngRNA spacer (TG PAM) |
| 48 | TTTTGGATGCAGAAGCTTTC (SEQ ID NO: 48) | PE3 ngRNA spacer (TG PAM) |
| 49 | TTTCTTGTGATTATCAGTTTGTTTTAGAGCTAGAA | PE3 ngRNA (TG PAM) |
| | ATAGCAAGTTAAAATAAGGCTAGTCCGTTATCAA | |
| | CTTGAAAAAGTGGCACCGAGTCGGTGC (SEQ ID | |
| | NO: 49) | |
| 50 | GGATGCAGAAGCTTTCTGTAGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 50) | |
| 51 | GAAGCTTTCTGTATGGGATCGTTTTAGAGCTAGA | PE3b ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 51) | |
| 53 | GGATGCAGAAGCTTTCTGTAGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 53) | |
| 54 | GAAGCTTTCTGTATGGGATCGTTTTAGAGCTAGA | PE3b ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 54) | |
| 55 | GTTTCTTGTGATTATCAGTTTGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 55) | |
| 57 | GGATGCAGAAGCTTTCTGTAGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 57) | |
| 58 | GAAGCTTTCTGTATGGGATCGTTTTAGAGCTAGA | PE3b ngRNA (AG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 58) | |
| 59 | GTTTCTTGTGATTATCAGTTTGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 59) | |

TABLE 27

| Sequence | | |
|---|---|---|
| Number | Sequence | Description |
| 1966 | ATGCAGAAGCTTTCTGT (SEQ ID NO: 1966) | PERNA spacer (ATGG; ATGGAT PAM) |
| 1967 | GATGCAGAAGCTTTCTGT (SEQ ID NO: 1967) | PEgRNA spacer (ATGG; ATGGAT PAM) |
| 1968 | GGATGCAGAAGCTTTCTGT (SEQ ID NO: 1968) | PEgRNA spacer (ATGG; ATGGAT PAM) |
| 134 | TGGATGCAGAAGCTTTCTGT (SEQ ID NO: 134) | PEgRNA spacer (ATGG; ATGGAT PAM) |
| 1969 | TTGGATGCAGAAGCTTTCTGT (SEQ ID NO: 1969) | PEgRNA spacer (ATGG; ATGGAT PAM) |
| 1970 | TTTGGATGCAGAAGCTTTCTGT (SEQ ID NO: 1970) | PEgRNA spacer (ATGG; ATGGAT PAM) |
| 1971 | GAAAG | PBS |
| 1972 | GAAAGC | PBS |
| 1973 | GAAAGCT | PBS |
| 1974 | GAAAGCTT | PBS |
| 1975 | GAAAGCTTC | PBS |
| 1976 | GAAAGCTTCT (SEQ ID NO: 1976) | PBS |
| 1977 | GAAAGCTTCTG (SEQ ID NO: 1977) | PBS |
| 1978 | GAAAGCTTCTGC (SEQ ID NO: 1978) | PBS |
| 1979 | GAAAGCTTCTGCA (SEQ ID NO: 1979) | PBS |
| 1980 | GAAAGCTTCTGCAT (SEQ ID NO: 1980) | PBS |
| 1981 | GAAAGCTTCTGCATC (SEQ ID NO: 1981) | PBS |
| 1982 | GAAAGCTTCTGCATCC (SEQ ID NO: 1982) | PBS |
| 1983 | GAAAGCTTCTGCATCCA (SEQ ID NO: 1983) | PBS |
| 1984 | GAAAGCTTCTGCATCCAA (SEQ ID NO: 1984) | PBS |
| 1985 | GAAAGCTTCTGCATCCAAA (SEQ ID NO: 1985) | PBS |
| 1986 | ATCCCATACA (SEQ ID NO: 1986) | RTT |
| 1987 | GATCCCATACA (SEQ ID NO: 1987) | RTT |
| 1988 | TGATCCCATACA (SEQ ID NO: 1988) | RTT |
| 1989 | CTGATCCCATACA (SEQ ID NO: 1989) | RTT |
| 1990 | CCTGATCCCATACA (SEQ ID NO: 1990) | RTT |
| 1991 | GCCTGATCCCATACA (SEQ ID NO: 1991) | RTT |
| 1992 | AGCCTGATCCCATACA (SEQ ID NO: 1992) | RTT |
| 1993 | AAGCCTGATCCCATACA (SEQ ID NO: 1993) | RTT |
| 1994 | GAAGCCTGATCCCATACA (SEQ ID NO: 1994) | RTT |
| 1995 | GGAAGCCTGATCCCATACA (SEQ ID NO: 1995) | RTT |
| 1996 | TGGAAGCCTGATCCCATACA (SEQ ID NO: 1996) | RTT |
| 1997 | GTGGAAGCCTGATCCCATACA (SEQ ID NO: 1997) | RTT |
| 1998 | AGTGGAAGCCTGATCCCATACA (SEQ ID NO: | RTT |
| | 1998) | |
| 1999 | AAGTGGAAGCCTGATCCCATACA (SEQ ID NO: | RTT |
| | 1999) | |
| 2000 | AAAGTGGAAGCCTGATCCCATACA (SEQ ID NO: | RTT |
| | 2000) | |
| 2001 | CAAAGTGGAAGCCTGATCCCATACA (SEQ ID NO: | RTT |
| | 2001) | |
| 2002 | CCAAAGTGGAAGCCTGATCCCATACA (SEQ ID | RTT |
| | NO: 2002) | |
| 2003 | TCCAAAGTGGAAGCCTGATCCCATACA (SEQ ID | RTT |
| | NO: 2003) | |
| 2004 | TTCCAAAGTGGAAGCCTGATCCCATACA (SEQ ID | RTT |
| | NO: 2004) | |
| 2005 | TTTCCAAAGTGGAAGCCTGATCCCATACA (SEQ | RTT |
| | ID NO: 2005) | |
| 2006 | GTTTCCAAAGTGGAAGCCTGATCCCATACA (SEQ | RTT |
| | ID NO: 2006) | |
| 2007 | GGTTTCCAAAGTGGAAGCCTGATCCCATACA | RTT |
| | (SEQ ID NO: 2007) | |
| 2008 | GGGTTTCCAAAGTGGAAGCCTGATCCCATACA | RTT |
| | (SEQ ID NO: 2008) | |
| 2009 | TGGGTTTCCAAAGTGGAAGCCTGATCCCATACA | RTT |
| | (SEQ ID NO: 2009) | |
| 2010 | CTGGGTTTCCAAAGTGGAAGCCTGATCCCATACA | RTT |
| | (SEQ ID NO: 2010) | |
| 2011 | GCTGGGTTTCCAAAGTGGAAGCCTGATCCCATAC | RTT |
| | A (SEQ ID NO: 2011) | |
| 2012 | TGCTGGGTTTCCAAAGTGGAAGCCTGATCCCATA | RTT |
| | CA (SEQ ID NO: 2012) | |
| 2013 | TTGCTGGGTTTCCAAAGTGGAAGCCTGATCCCAT | RTT |
| | ACA (SEQ ID NO: 2013) | |
| 2014 | CTTGCTGGGTTTCCAAAGTGGAAGCCTGATCCCA | RTT |
| | TACA (SEQ ID NO: 2014) | |
| 2015 | TCTTGCTGGGTTTCCAAAGTGGAAGCCTGATCCC | RTT |
| | ATACA (SEQ ID NO: 2015) | |
| 2016 | GTCTTGCTGGGTTTCCAAAGTGGAAGCCTGATCC | RTT |
| | CATACA (SEQ ID NO: 2016) | |
| 480 | AAGGCTTCATACATCTTCCT (SEQ ID NO: 480) | PE3 ngRNA spacer (TAGG PAM) |
| 64 | ACGTGAAGACAGGTGTCTTG (SEQ ID NO: 64) | PE3 ngRNA spacer (CTGG; CTGGGT PAM) |
| 482 | CACTACTTACCATCTCTTTC (SEQ ID NO: 482) | PE3 ngRNA spacer (AAGG PAM) |
| 412 | CAGGTGTCTTGCTGGGTTTC (SEQ ID NO: 412) | PE3 ngRNA spacer (CAAAGT PAM) |
| 203 | CGTGAAGACAGGTGTCTTGC (SEQ ID NO: 203) | PE3 ngRNA spacer (TGGG PAM) |
| 413 | CTGGGTTTCCAAAGTGGAAG (SEQ ID NO: 413) | PE3 ngRNA spacer (CCTGAT PAM) |
| 485 | GGAACTGAGCCACGTGAAGA (SEQ ID NO: 485) | PE3 ngRNA spacer (CAGG PAM) |
| 542 | TACATCTTCCTTAGGAACTC (SEQ ID NO: 542) | PE3 ngRNA spacer (CTGG PAM) |
| 414 | TGGAACTGAGCCACGTGAAG (SEQ ID NO: 414) | PE3 ngRNA spacer (ACAGGT PAM) |
| 543 | TGTCTTGCTGGGTTTCCAAA (SEQ ID NO: 543) | PE3 ngRNA spacer (GTGG PAM) |
| 325 | CGTGAAGACAGGTGTCTTGCGTTTTAGAGCTAGA | PE3 ngRNA (TGGG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 333 | GCGTGAAGACAGGTGTCTTGCGTTTTAGAGCTAG | PE3 ngRNA (TGGG PAM),, contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 340 | GCGTGAAGACAGGTGTCTTGCGTTTTAGAGCTAG | PE3 ngRNA (TGGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |

TABLE 28

| Sequence | | |
|---|---|---|
| Number | Sequence | Description |
| 2017 | CTTGCTGGGTTTCCAAA (SEQ ID NO: 2017) | PEgRNA spacer (GTGG PAM) |
| 2018 | TCTTGCTGGGTTTCCAAA (SEQ ID NO: 2018) | PEgRNA spacer (GTGG PAM) |
| 2019 | GTCTTGCTGGGTTTCCAAA (SEQ ID NO: 2019) | PEgRNA spacer (GTGG PAM) |
| 543 | TGTCTTGCTGGGTTTCCAAA (SEQ ID NO: 543) | PERNA spacer (GTGG PAM) |
| 2020 | GTGTCTTGCTGGGTTTCCAAA (SEQ ID NO: 2020) | PEgRNA spacer (GTGG PAM) |
| 2021 | GGTGTCTTGCTGGGTTTCCAAA (SEQ ID NO: 2021) | PEgRNA spacer (GTGG PAM) |
| 2022 | GGAAA | PBS |
| 2023 | GGAAAC | PBS |
| 2024 | GGAAACC | PBS |
| 2025 | GGAAACCC | PBS |
| 2026 | GGAAACCCA | PBS |
| 2027 | GGAAACCCAG (SEQ ID NO: 2027) | PBS |
| 2028 | GGAAACCCAGC (SEQ ID NO: 2028) | PBS |
| 2029 | GGAAACCCAGCA (SEQ ID NO: 2029) | PBS |
| 2030 | GGAAACCCAGCAA (SEQ ID NO: 2030) | PBS |
| 2031 | GGAAACCCAGCAAG (SEQ ID NO: 2031) | PBS |
| 2032 | GGAAACCCAGCAAGA (SEQ ID NO: 2032) | PBS |
| 2033 | GGAAACCCAGCAAGAC (SEQ ID NO: 2033) | PBS |
| 2034 | GGAAACCCAGCAAGACA (SEQ ID NO: 2034) | PBS |
| 2035 | GGAAACCCAGCAAGACAC (SEQ ID NO: 2035) | PBS |
| 2036 | GGAAACCCAGCAAGACACC (SEQ ID NO: 2036) | PBS |
| 2037 | GATCAGGCTAGCACTTT (SEQ ID NO: 2037) | RTT*1, 2 (GTGG-to-GTGC PAM silencing |
| | | edit) |
| 2038 | GATCAGGCTAGTACTTT (SEQ ID NO: 2038) | RTT*1, 3 (GTGG-to-GTAC PAM silencing |
| | | edit) |
| 2039 | GATCAGGCTTCAACTTT (SEQ ID NO: 2039) | RTT*1, 4 (GTGG-to-GTTG PAM silencing |
| | | edit) |
| 2040 | GATCAGGCTTCCACTTT (SEQ ID NO: 2040) | RTT*1, 5 |
| 2041 | GATCAGGCTTCGACTTT (SEQ ID NO: 2041) | RTT*1, 6 (GTGG-to-GTCG PAM silencing |
| | | edit) |
| 2042 | GATCAGGCTTCTACTTT (SEQ ID NO: 2042) | RTT*1, 7 (GTGG-to-GTAG PAM silencing |
| | | edit) |
| 2043 | GGATCAGGCTAGCACTTT (SEQ ID NO: 2043) | RTT*1, 2 (GTGG-to-GTGC PAM silencing |
| | | edit) |
| 2044 | GGATCAGGCTAGTACTTT (SEQ ID NO: 2044) | RTT*1, 3 (GTGG-to-GTAC PAM silencing |
| | | edit) |
| 2045 | GGATCAGGCTTCAACTTT (SEQ ID NO: 2045) | RTT*1, 4 (GTGG-to-GTTG PAM silencing |
| | | edit) |
| 2046 | GGATCAGGCTTCCACTTT (SEQ ID NO: 2046) | RTT*1, 5 |
| 2047 | GGATCAGGCTTCGACTTT (SEQ ID NO: 2047) | RTT*1, 6 (GTGG-to-GTCG PAM silencing |
| | | edit) |
| 2048 | GGATCAGGCTTCTACTTT (SEQ ID NO: 2048) | RTT*1, 7 (GTGG-to-GTAG PAM silencing |
| | | edit) |
| 2049 | GGGATCAGGCTAGCACTTT (SEQ ID NO: 2049) | RTT*1, 2 (GTGG-to-GTGC PAM silencing |
| | | edit) |
| 2050 | GGGATCAGGCTAGTACTTT (SEQ ID NO: 2050) | RTT*1, 3 (GTGG-to-GTAC PAM silencing |
| | | edit) |
| 2051 | GGGATCAGGCTTCAACTTT (SEQ ID NO: 2051) | RTT*1, 4 (GTGG-to-GTTG PAM silencing |
| | | edit) |
| 2052 | GGGATCAGGCTTCCACTTT (SEQ ID NO: 2052) | RTT*1, 5 |
| 2053 | GGGATCAGGCTTCGACTTT (SEQ ID NO: 2053) | RTT*1, 6 (GTGG-to-GTCG PAM silencing |
| | | edit) |
| 2054 | GGGATCAGGCTTCTACTTT (SEQ ID NO: 2054) | RTT*1, 7 (GTGG-to-GTAG PAM silencing |
| | | edit) |
| 1821 | TGGGATCAGGCTAGCACTTT (SEQ ID NO: 1821) | RTT*1, 2 (GTGG-to-GTGC PAM silencing |
| | | edit) |
| 1399 | TGGGATCAGGCTAGTACTTT (SEQ ID NO: 1399) | RTT*1, 3 (GTGG-to-GTAC PAM silencing |
| | | edit) |
| 1400 | TGGGATCAGGCTTCAACTTT (SEQ ID NO: 1400) | RTT*1, 4 (GTGG-to-GTTG PAM silencing |
| | | edit) |
| 43 | TGGGATCAGGCTTCCACTTT (SEQ ID NO: 43) | RTT*1, 5 |
| 1401 | TGGGATCAGGCTTCGACTTT (SEQ ID NO: 1401) | RTT*1, 6 (GTGG-to-GTCG PAM silencing |
| | | edit) |
| 1402 | TGGGATCAGGCTTCTACTTT (SEQ ID NO: 1402) | RTT*1, 7 (GTGG-to-GTAG PAM silencing |
| | | edit) |
| 2055 | ATGGGATCAGGCTAGCACTTT (SEQ ID NO: 2055) | RTT*1, 2 (GTGG-to-GTGC PAM silencing |
| | | edit) |
| 2056 | ATGGGATCAGGCTAGTACTTT (SEQ ID NO: 2056) | RTT*1, 3 (GTGG-to-GTAC PAM silencing |
| | | edit) |
| 2057 | ATGGGATCAGGCTTCAACTTT (SEQ ID NO: 2057) | RTT*1, 4 (GTGG-to-GTTG PAM silencing |
| | | edit) |
| 2058 | ATGGGATCAGGCTTCCACTTT (SEQ ID NO: 2058) | RTT*1, 5 |
| 2059 | ATGGGATCAGGCTTCGACTTT (SEQ ID NO: 2059) | RTT*1, 6 (GTGG-to-GTCG PAM silencing |
| | | edit) |
| 2060 | ATGGGATCAGGCTTCTACTTT (SEQ ID NO: 2060) | RTT*1, 7 (GTGG-to-GTAG PAM silencing |
| | | edit) |
| 2061 | TATGGGATCAGGCTAGCACTTT (SEQ ID NO: 2061) | RTT*1, 2 (GTGG-to-GTGC PAM silencing |
| | | edit) |
| 2062 | TATGGGATCAGGCTAGTACTTT (SEQ ID NO: 2062) | RTT*1, 3 (GTGG-to-GTAC PAM silencing |
| | | edit) |
| 2063 | TATGGGATCAGGCTTCAACTTT (SEQ ID NO: 2063) | RTT*1, 4 (GTGG-to-GTTG PAM silencing |
| | | edit) |
| 2064 | TATGGGATCAGGCTTCCACTTT (SEQ ID NO: 2064) | RTT*1, 5 |
| 2065 | TATGGGATCAGGCTTCGACTTT (SEQ ID NO: 2065) | RTT*1, 6 (GTGG-to-GTCG PAM silencing |
| | | edit) |
| 2066 | TATGGGATCAGGCTTCTACTTT (SEQ ID NO: 2066) | RTT*1, 7 (GTGG-to-GTAG PAM silencing |
| | | edit) |
| 2067 | GTATGGGATCAGGCTAGCACTTT (SEQ ID NO: | RTT*1, 2 (GTGG-to-GTGC PAM silencing |
| | 2067) | edit) |
| 2068 | GTATGGGATCAGGCTAGTACTTT (SEQ ID NO: | RTT*1, 3 (GTGG-to-GTAC PAM silencing |
| | 2068) | edit) |
| 2069 | GTATGGGATCAGGCTTCAACTTT (SEQ ID NO: | RTT*1, 4 (GTGG-to-GTTG PAM silencing |
| | 2069) | edit) |
| 2070 | GTATGGGATCAGGCTTCCACTTT (SEQ ID NO: | RTT*1, 5 |
| | 2070) | |
| 2071 | GTATGGGATCAGGCTTCGACTTT (SEQ ID NO: | RTT*1, 6 (GTGG-to-GTCG PAM silencing |
| | 2071) | edit) |
| 2072 | GTATGGGATCAGGCTTCTACTTT (SEQ ID NO: | RTT*1, 7 (GTGG-to-GTAG PAM silencing |
| | 2072) | edit) |
| 2073 | TGTATGGGATCAGGCTAGCACTTT (SEQ ID NO: | RTT*1, 2 (GTGG-to-GTGC PAM silencing |
| | 2073) | edit) |
| 2074 | TGTATGGGATCAGGCTAGTACTTT (SEQ ID NO: | RTT*1, 3 (GTGG-to-GTAC PAM silencing |
| | 2074) | edit) |
| 2075 | TGTATGGGATCAGGCTTCAACTTT (SEQ ID NO: | RTT*1, 4 (GTGG-to-GTTG PAM silencing |
| | 2075) | edit) |
| 2076 | TGTATGGGATCAGGCTTCCACTTT (SEQ ID NO: | RTT*1, 5 |
| | 2076) | |
| 2077 | TGTATGGGATCAGGCTTCGACTTT (SEQ ID NO: | RTT*1, 6 (GTGG-to-GTCG PAM silencing |
| | 2077) | edit) |
| 2078 | TGTATGGGATCAGGCTTCTACTTT (SEQ ID NO: | RTT*1, 7 (GTGG-to-GTAG PAM silencing |
| | 2078) | edit) |
| 2079 | CTGTATGGGATCAGGCTAGCACTTT (SEQ ID NO: | RTT*1, 2 (GTGG-to-GTGC PAM silencing |
| | 2079) | edit) |
| 2080 | CTGTATGGGATCAGGCTAGTACTTT (SEQ ID NO: | RTT*1, 3 (GTGG-to-GTAC PAM silencing |
| | 2080) | edit) |
| 2081 | CTGTATGGGATCAGGCTTCAACTTT (SEQ ID NO: | RTT*1, 4 (GTGG-to-GTTG PAM silencing |
| | 2081) | edit) |
| 2082 | CTGTATGGGATCAGGCTTCCACTTT (SEQ ID NO: | RTT*1, 5 |
| | 2082) | |
| 2083 | CTGTATGGGATCAGGCTTCGACTTT (SEQ ID NO: | RTT*1, 6 (GTGG-to-GTCG PAM silencing |
| | 2083) | edit) |
| 2084 | CTGTATGGGATCAGGCTTCTACTTT (SEQ ID NO: | RTT*1, 7 (GTGG-to-GTAG PAM silencing |
| | 2084) | edit) |
| 2085 | TCTGTATGGGATCAGGCTAGCACTTT (SEQ ID NO: | RTT*1, 2 (GTGG-to-GTGC PAM silencing |
| | 2085) | edit) |
| 2086 | TCTGTATGGGATCAGGCTAGTACTTT (SEQ ID NO: | RTT*1, 3 (GTGG-to-GTAC PAM silencing |
| | 2086) | edit) |
| 2087 | TCTGTATGGGATCAGGCTTCAACTTT (SEQ ID NO: | RTT*1, 4 (GTGG-to-GTTG PAM silencing |
| | 2087) | edit) |
| 2088 | TCTGTATGGGATCAGGCTTCCACTTT (SEQ ID NO: | RTT*1, 5 |
| | 2088) | |
| 2089 | TCTGTATGGGATCAGGCTTCGACTTT (SEQ ID NO: | RTT*1, 6 (GTGG-to-GTCG PAM silencing |
| | 2089) | edit) |
| 2090 | TCTGTATGGGATCAGGCTTCTACTTT (SEQ ID NO: | RTT*1, 7 (GTGG-to-GTAG PAM silencing |
| | 2090) | edit) |
| 2091 | TTCTGTATGGGATCAGGCTAGCACTTT (SEQ ID | RTT*1, 2 (GTGG-to-GTGC PAM silencing |
| | NO: 2091) | edit) |
| 2092 | TTCTGTATGGGATCAGGCTAGTACTTT (SEQ ID | RTT*1, 3 (GTGG-to-GTAC PAM silencing |
| | NO: 2092) | edit) |
| 2093 | TTCTGTATGGGATCAGGCTTCAACTTT (SEQ ID | RTT*1, 4 (GTGG-to-GTTG PAM silencing |
| | NO: 2093) | edit) |
| 2094 | TTCTGTATGGGATCAGGCTTCCACTTT (SEQ ID | RTT*1, 5 |
| | NO: 2094) | |
| 2095 | TTCTGTATGGGATCAGGCTTCGACTTT (SEQ ID | RTT*1, 6 (GTGG-to-GTCG PAM silencing |
| | NO: 2095) | edit) |
| 2096 | TTCTGTATGGGATCAGGCTTCTACTTT (SEQ ID | RTT*1, 7 (GTGG-to-GTAG PAM silencing |
| | NO: 2096) | edit) |
| 2097 | TTTCTGTATGGGATCAGGCTAGCACTTT (SEQ ID | RTT*1, 2 (GTGG-to-GTGC PAM silencing |
| | NO: 2097) | edit) |
| 2098 | TTTCTGTATGGGATCAGGCTAGTACTTT (SEQ ID | RTT*1, 3 (GTGG-to-GTAC PAM silencing |
| | NO: 2098) | edit) |
| 2099 | TTTCTGTATGGGATCAGGCTTCAACTTT (SEQ ID | RTT*1, 4 (GTGG-to-GTTG PAM silencing |
| | NO: 2099) | edit) |
| 2100 | TTTCTGTATGGGATCAGGCTTCCACTTT (SEQ ID | RTT*1, 5 |
| | NO: 2100) | |
| 2101 | TTTCTGTATGGGATCAGGCTTCGACTTT (SEQ ID | RTT*1, 6 (GTGG-to-GTCG PAM silencing |
| | NO: 2101) | edit) |
| 2102 | TTTCTGTATGGGATCAGGCTTCTACTTT (SEQ ID | RTT*1, 7 (GTGG-to-GTAG PAM silencing |
| | NO: 2102) | edit) |
| 2103 | CTTTCTGTATGGGATCAGGCTAGCACTTT (SEQ ID | RTT*1, 2 (GTGG-to-GTGC PAM silencing |
| | NO: 2103) | edit) |
| 2104 | CTTTCTGTATGGGATCAGGCTAGTACTTT (SEQ ID | RTT*1, 3 (GTGG-to-GTAC PAM silencing |
| | NO: 2104) | edit) |
| 2105 | CTTTCTGTATGGGATCAGGCTTCAACTTT (SEQ ID | RTT*1, 4 (GTGG-to-GTTG PAM silencing |
| | NO: 2105) | edit) |
| 2106 | CTTTCTGTATGGGATCAGGCTTCCACTTT (SEQ ID | RTT*1, 5 |
| | NO: 2106) | |
| 2107 | CTTTCTGTATGGGATCAGGCTTCGACTTT (SEQ ID | RTT*1, 6 (GTGG-to-GTCG PAM silencing |
| | NO: 2107) | edit) |
| 2108 | CTTTCTGTATGGGATCAGGCTTCTACTTT (SEQ ID | RTT*1, 7 (GTGG-to-GTAG PAM silencing |
| | NO: 2108) | edit) |
| 2109 | GCTTTCTGTATGGGATCAGGCTAGCACTTT (SEQ | RTT*1, 2 (GTGG-to-GTGC PAM silencing |
| | ID NO: 2109) | edit) |
| 2110 | GCTTTCTGTATGGGATCAGGCTAGTACTTT (SEQ | RTT*1, 3 (GTGG-to-GTAC PAM silencing |
| | ID NO: 2110) | edit) |
| 2111 | GCTTTCTGTATGGGATCAGGCTTCAACTTT (SEQ | RTT*1, 4 (GTGG-to-GTTG PAM silencing |
| | ID NO: 2111) | edit) |
| 2112 | GCTTTCTGTATGGGATCAGGCTTCCACTTT (SEQ | RTT*1, 5 |
| | ID NO: 2112) | |
| 2113 | GCTTTCTGTATGGGATCAGGCTTCGACTTT (SEQ | RTT*1, 6 (GTGG-to-GTCG PAM silencing |
| | ID NO: 2113) | edit) |
| 2114 | GCTTTCTGTATGGGATCAGGCTTCTACTTT (SEQ | RTT*1, 7 (GTGG-to-GTAG PAM silencing |
| | ID NO: 2114) | edit) |
| 2115 | AGCTTTCTGTATGGGATCAGGCTAGCACTTT | RTT*1, 2 (GTGG-to-GTGC PAM silencing |
| | (SEQ ID NO: 2115) | edit) |
| 2116 | AGCTTTCTGTATGGGATCAGGCTAGTACTTT (SEQ | RTT*1, 3 (GTGG-to-GTAC PAM silencing |
| | ID NO: 2116) | edit) |
| 2117 | AGCTTTCTGTATGGGATCAGGCTTCAACTTT (SEQ | RTT*1, 4 (GTGG-to-GTTG PAM silencing |
| | ID NO: 2117) | edit) |
| 2118 | AGCTTTCTGTATGGGATCAGGCTTCCACTTT (SEQ | RTT*1, 5 |
| | ID NO: 2118) | |
| 2119 | AGCTTTCTGTATGGGATCAGGCTTCGACTTT (SEQ | RTT*1, 6 (GTGG-to-GTCG PAM silencing |
| | ID NO: 2119) | edit) |
| 2120 | AGCTTTCTGTATGGGATCAGGCTTCTACTTT (SEQ | RTT*1, 7 (GTGG-to-GTAG PAM silencing |
| | ID NO: 2120) | edit) |
| 2121 | AAGCTTTCTGTATGGGATCAGGCTAGCACTTT | RTT*1, 2 (GTGG-to-GTGC PAM silencing |
| | (SEQ ID NO: 2121) | edit) |
| 2122 | AAGCTTTCTGTATGGGATCAGGCTAGTACTTT | RTT*1, 3 (GTGG-to-GTAC PAM silencing |
| | (SEQ ID NO: 2122) | edit) |
| 2123 | AAGCTTTCTGTATGGGATCAGGCTTCAACTTT | RTT*1, 4 (GTGG-to-GTTG PAM silencing |
| | (SEQ ID NO: 2123) | edit) |
| 2124 | AAGCTTTCTGTATGGGATCAGGCTTCCACTTT | RTT*1, 5 |
| | (SEQ ID NO: 2124) | |
| 2125 | AAGCTTTCTGTATGGGATCAGGCTTCGACTTT | RTT*1, 6 (GTGG-to-GTCG PAM silencing |
| | (SEQ ID NO: 2125) | edit) |
| 2126 | AAGCTTTCTGTATGGGATCAGGCTTCTACTTT | RTT*1, 7 (GTGG-to-GTAG PAM silencing |
| | (SEQ ID NO: 2126) | edit) |
| 2127 | GAAGCTTTCTGTATGGGATCAGGCTAGCACTTT | RTT*1, 2 (GTGG-to-GTGC PAM silencing |
| | (SEQ ID NO: 2127) | edit) |
| 2128 | GAAGCTTTCTGTATGGGATCAGGCTAGTACTTT | RTT*1, 3 (GTGG-to-GTAC PAM silencing |
| | (SEQ ID NO: 2128) | edit) |
| 2129 | GAAGCTTTCTGTATGGGATCAGGCTTCAACTTT | RTT*1, 4 (GTGG-to-GTTG PAM silencing |
| | (SEQ ID NO: 2129) | edit) |
| 2130 | GAAGCTTTCTGTATGGGATCAGGCTTCCACTTT | RTT*1, 5 |
| | (SEQ ID NO: 2130) | |
| 2131 | GAAGCTTTCTGTATGGGATCAGGCTTCGACTTT | RTT*1, 6 (GTGG-to-GTCG PAM silencing |
| | (SEQ ID NO: 2131) | edit) |
| 2132 | GAAGCTTTCTGTATGGGATCAGGCTTCTACTTT | RTT*1, 7 (GTGG-to-GTAG PAM silencing |
| | (SEQ ID NO: 2132) | edit) |
| 2133 | AGAAGCTTTCTGTATGGGATCAGGCTAGCACTTT | RTT*1, 2 (GTGG-to-GTGC PAM silencing |
| | (SEQ ID NO: 2133) | edit) |
| 2134 | AGAAGCTTTCTGTATGGGATCAGGCTAGTACTTT | RTT*1, 3 (GTGG-to-GTAC PAM silencing |
| | (SEQ ID NO: 2134) | edit) |
| 2135 | AGAAGCTTTCTGTATGGGATCAGGCTTCAACTTT | RTT*1, 4 (GTGG-to-GTTG PAM silencing |
| | (SEQ ID NO: 2135) | edit) |
| 2136 | AGAAGCTTTCTGTATGGGATCAGGCTTCCACTTT | RTT*1, 5 |
| | (SEQ ID NO: 2136) | |
| 2137 | AGAAGCTTTCTGTATGGGATCAGGCTTCGACTTT | RTT*1, 6 (GTGG-to-GTCG PAM silencing |
| | (SEQ ID NO: 2137) | edit) |
| 2138 | AGAAGCTTTCTGTATGGGATCAGGCTTCTACTTT | RTT*1, 7 (GTGG-to-GTAG PAM silencing |
| | (SEQ ID NO: 2138) | edit) |
| 2139 | CAGAAGCTTTCTGTATGGGATCAGGCTAGCACTT | RTT*1, 2 (GTGG-to-GTGC PAM silencing |
| | T (SEQ ID NO: 2139) | edit) |
| 2140 | CAGAAGCTTTCTGTATGGGATCAGGCTAGTACTT | RTT*1, 3 (GTGG-to-GTAC PAM silencing |
| | T (SEQ ID NO: 2140) | edit) |
| 2141 | CAGAAGCTTTCTGTATGGGATCAGGCTTCAACTT | RTT*1, 4 (GTGG-to-GTTG PAM silencing |
| | T (SEQ ID NO: 2141) | edit) |
| 2142 | CAGAAGCTTTCTGTATGGGATCAGGCTTCCACTT | RTT*1, 5 |
| | T (SEQ ID NO: 2142) | |
| 2143 | CAGAAGCTTTCTGTATGGGATCAGGCTTCGACTT | RTT*1, 6 (GTGG-to-GTCG PAM silencing |
| | T (SEQ ID NO: 2143) | edit) |
| 2144 | CAGAAGCTTTCTGTATGGGATCAGGCTTCTACTT | RTT*1, 7 (GTGG-to-GTAG PAM silencing |
| | T (SEQ ID NO: 2144) | edit) |
| 2145 | GCAGAAGCTTTCTGTATGGGATCAGGCTAGCACT | RTT*1, 2 (GTGG-to-GTGC PAM silencing |
| | TT (SEQ ID NO: 2145) | edit) |
| 2146 | GCAGAAGCTTTCTGTATGGGATCAGGCTAGTACT | RTT*1, 3 (GTGG-to-GTAC PAM silencing |
| | TT (SEQ ID NO: 2146) | edit) |
| 2147 | GCAGAAGCTTTCTGTATGGGATCAGGCTTCAACT | RTT*1, 4 (GTGG-to-GTTG PAM silencing |
| | TT (SEQ ID NO: 2147) | edit) |
| 2148 | GCAGAAGCTTTCTGTATGGGATCAGGCTTCCACT | RTT*1, 5 |
| | TT (SEQ ID NO: 2148) | |
| 2149 | GCAGAAGCTTTCTGTATGGGATCAGGCTTCGACT | RTT*1, 6 (GTGG-to-GTCG PAM silencing |
| | TT (SEQ ID NO: 2149) | edit) |
| 2150 | GCAGAAGCTTTCTGTATGGGATCAGGCTTCTACT | RTT*1, 7 (GTGG-to-GTAG PAM silencing |
| | TT (SEQ ID NO: 2150) | edit) |
| 2151 | TGCAGAAGCTTTCTGTATGGGATCAGGCTAGCAC | RTT*1, 2 (GTGG-to-GTGC PAM silencing |
| | TTT (SEQ ID NO: 2151) | edit) |
| 2152 | TGCAGAAGCTTTCTGTATGGGATCAGGCTAGTAC | RTT*1, 3 (GTGG-to-GTAC PAM silencing |
| | TTT (SEQ ID NO: 2152) | edit) |
| 2153 | TGCAGAAGCTTTCTGTATGGGATCAGGCTTCAAC | RTT*1, 4 (GTGG-to-GTTG PAM silencing |
| | TTT (SEQ ID NO: 2153) | edit) |
| 2154 | TGCAGAAGCTTTCTGTATGGGATCAGGCTTCCAC | RTT*1, 5 |
| | TTT (SEQ ID NO: 2154) | |
| 2155 | TGCAGAAGCTTTCTGTATGGGATCAGGCTTCGAC | RTT*1, 6 (GTGG-to-GTCG PAM silencing |
| | TTT (SEQ ID NO: 2155) | edit) |
| 2156 | TGCAGAAGCTTTCTGTATGGGATCAGGCTTCTAC | RTT*1, 7 (GTGG-to-GTAG PAM silencing |
| | TTT (SEQ ID NO: 2156) | edit) |
| 2157 | ATGCAGAAGCTTTCTGTATGGGATCAGGCTAGCA | RTT*1, 2 (GTGG-to-GTGC PAM silencing |
| | CTTT (SEQ ID NO: 2157) | edit) |
| 2158 | ATGCAGAAGCTTTCTGTATGGGATCAGGCTAGTA | RTT*1, 3 (GTGG-to-GTAC PAM silencing |
| | CTTT (SEQ ID NO: 2158) | edit) |
| 2159 | ATGCAGAAGCTTTCTGTATGGGATCAGGCTTCAA | RTT*1, 4 (GTGG-to-GTTG PAM silencing |
| | CTTT (SEQ ID NO: 2159) | edit) |
| 2160 | ATGCAGAAGCTTTCTGTATGGGATCAGGCTTCCA | RTT*1, 5 |
| | CTTT (SEQ ID NO: 2160) | |
| 2161 | ATGCAGAAGCTTTCTGTATGGGATCAGGCTTCGA | RTT*1, 6 (GTGG-to-GTCG PAM silencing |
| | CTTT (SEQ ID NO: 2161) | edit) |
| 2162 | ATGCAGAAGCTTTCTGTATGGGATCAGGCTTCTA | RTT*1, 7 (GTGG-to-GTAG PAM silencing |
| | CTTT (SEQ ID NO: 2162) | edit) |
| 2163 | GATGCAGAAGCTTTCTGTATGGGATCAGGCTAGC | RTT*1, 2 (GTGG-to-GTGC PAM silencing |
| | ACTTT (SEQ ID NO: 2163) | edit) |
| 2164 | GATGCAGAAGCTTTCTGTATGGGATCAGGCTAGT | RTT*1, 3 (GTGG-to-GTAC PAM silencing |
| | ACTTT (SEQ ID NO: 2164) | edit) |
| 2165 | GATGCAGAAGCTTTCTGTATGGGATCAGGCTTCA | RTT*1, 4 (GTGG-to-GTTG PAM silencing |
| | ACTTT (SEQ ID NO: 2165) | edit) |
| 2166 | GATGCAGAAGCTTTCTGTATGGGATCAGGCTTCC | RTT*1, 5 |
| | ACTTT (SEQ ID NO: 2166) | |
| 2167 | GATGCAGAAGCTTTCTGTATGGGATCAGGCTTCG | RTT*1, 6 (GTGG-to-GTCG PAM silencing |
| | ACTTT (SEQ ID NO: 2167) | edit) |
| 2168 | GATGCAGAAGCTTTCTGTATGGGATCAGGCTTCT | RTT*1, 7 (GTGG-to-GTAG PAM silencing |
| | ACTTT (SEQ ID NO: 2168) | edit) |
| 2169 | GGATGCAGAAGCTTTCTGTATGGGATCAGGCTAG | RTT*1, 2 (GTGG-to-GTGC PAM silencing |
| | CACTTT (SEQ ID NO: 2169) | edit) |
| 2170 | GGATGCAGAAGCTTTCTGTATGGGATCAGGCTAG | RTT*1, 3 (GTGG-to-GTAC PAM silencing |
| | TACTTT (SEQ ID NO: 2170) | edit) |
| 2171 | GGATGCAGAAGCTTTCTGTATGGGATCAGGCTTC | RTT*1, 4 (GTGG-to-GTTG PAM silencing |
| | AACTTT (SEQ ID NO: 2171) | edit) |
| 2172 | GGATGCAGAAGCTTTCTGTATGGGATCAGGCTTC | RTT*1, 5 |
| | CACTTT (SEQ ID NO: 2172) | |
| 2173 | GGATGCAGAAGCTTTCTGTATGGGATCAGGCTTC | RTT*1, 6 (GTGG-to-GTCG PAM silencing |
| | GACTTT (SEQ ID NO: 2173) | edit) |
| 2174 | GGATGCAGAAGCTTTCTGTATGGGATCAGGCTTC | RTT*1, 7 (GTGG-to-GTAG PAM silencing |
| | TACTTT (SEQ ID NO: 2174) | edit) |
| 126 | ACCTTTTCGCTTTTTCCAAG (SEQ ID NO: 126) | PE3 ngRNA spacer (ATGG PAM) |
| 127 | AGAAGCTTTCTGTATGGGAT (SEQ ID NO: 127) | PE3b*1 ngRNA spacer (CAGG PAM) |
| 131 | CTTTCTTGTGATTATCAGTT (SEQ ID NO: 131) | PE3 ngRNA spacer (TTGG PAM) |
| 1227 | GTGCTGGACAGGCTGCTGTG (SEQ ID NO: 1227) | PE3 ngRNA spacer (AAGG PAM) |
| 1759 | TATGGGATCAGGCTAGCACT (SEQ ID NO: 1759) | PE3b*2 ngRNA spacer (TTGG PAM) |
| 1760 | TATGGGATCAGGCTAGTACT (SEQ ID NO: 1760) | PE3b*3 ngRNA spacer (TTGG PAM) |
| 2175 | TATGGGATCAGGCTTCAACT (SEQ ID NO: 2175) | PE3b*4 ngRNA spacer (TTGG PAM) |
| 132 | TATGGGATCAGGCTTCCACT (SEQ ID NO: 132) | PE3b*5 ngRNA spacer (TTGG PAM) |
| 2176 | TATGGGATCAGGCTTCGACT (SEQ ID NO: 2176) | PE3b*6 ngRNA spacer (TTGG PAM) |
| 2177 | TATGGGATCAGGCTTCTACT (SEQ ID NO: 2177) | PE3b*7 ngRNA spacer (TTGG PAM) |
| 1113 | TGCTGGACAGGCTGCTGTGA (SEQ ID NO: 1113) | PE3 ngRNA spacer (AGGG PAM) |
| 134 | TGGATGCAGAAGCTTTCTGT (SEQ ID NO: 134) | PE3 ngRNA spacer (ATGG PAM) |

TABLE 29

| Sequence | | |
|---|---|---|
| Number | Sequence | Description |
| 2178 | TCAGTTTTGGATGCAGA (SEQ ID NO: 2178) | PEgRNA spacer (AG PAM) |
| 2179 | ATCAGTTTTGGATGCAGA (SEQ ID NO: 2179) | PEgRNA spacer (AG PAM) |
| 2180 | TATCAGTTTTGGATGCAGA (SEQ ID NO: 2180) | PEgRNA spacer (AG PAM) |
| 44 | TTATCAGTTTTGGATGCAGA (SEQ ID NO: 44) | PERNA spacer (AG PAM) |
| 2181 | ATTATCAGTTTTGGATGCAGA (SEQ ID NO: 2181) | PEgRNA spacer (AG PAM) |
| 2182 | GATTATCAGTTTTGGATGCAGA (SEQ ID NO: 2182) | PEgRNA spacer (AG PAM) |
| 2183 | GCATC | PBS |
| 2184 | GCATCC | PBS |
| 2185 | GCATCCA | PBS |
| 2186 | GCATCCAA | PBS |
| 2187 | GCATCCAAA | PBS |
| 2188 | GCATCCAAAA (SEQ ID NO: 2188) | PBS |
| 2189 | GCATCCAAAAC (SEQ ID NO: 2189) | PBS |
| 2190 | GCATCCAAAACT (SEQ ID NO: 2190) | PBS |
| 2191 | GCATCCAAAACTG (SEQ ID NO: 2191) | PBS |
| 2192 | GCATCCAAAACTGA (SEQ ID NO: 2192) | PBS |
| 2193 | GCATCCAAAACTGAT (SEQ ID NO: 2193) | PBS |
| 2194 | GCATCCAAAACTGATA (SEQ ID NO: 2194) | PBS |
| 2195 | GCATCCAAAACTGATAA (SEQ ID NO: 2195) | PBS |
| 2196 | GCATCCAAAACTGATAAT (SEQ ID NO: 2196) | PBS |
| 2197 | GCATCCAAAACTGATAATC (SEQ ID NO: 2197) | PBS |
| 2198 | CCCATACAGAAAGCTTCT (SEQ ID NO: 2198) | RTT*1, 2, 4 |
| 2199 | CCCATACAGAAAGTTTCT (SEQ ID NO: 2199) | RTT*1, 3, 4 (AG-to-AA PAM silencing edit) |
| 2200 | TCCCATACAGAAAGCTTCT (SEQ ID NO: 2200) | RTT*1, 2, 4 |
| 2201 | TCCCATACAGAAAGTTTCT (SEQ ID NO: 2201) | RTT*1, 3, 4 (AG-to-AA PAM silencing edit) |
| 2202 | ATCCCATACAGAAAGCTTCT (SEQ ID NO: 2202) | RTT*1, 2, 4 |
| 2203 | ATCCCATACAGAAAGTTTCT (SEQ ID NO: 2203) | RTT*1, 3, 4 (AG-to-AA PAM silencing edit) |
| 2204 | GATCCCATACAGAAAGCTTCT (SEQ ID NO: 2204) | RTT*1, 2, 4 |
| 2205 | GATCCCATACAGAAAGTTTCT (SEQ ID NO: 2205) | RTT*1, 3, 4 (AG-to-AA PAM silencing edit) |
| 2206 | TGATCCCATACAGAAAGCTTCT (SEQ ID NO: 2206) | RTT*1, 2, 4 |
| 2207 | TGATCCCATACAGAAAGTTTCT (SEQ ID NO: 2207) | RTT*1, 3, 4 (AG-to-AA PAM silencing edit) |
| 2208 | CTGATCCCATACAGAAAGCTTCT (SEQ ID NO: | RTT*1, 2, 4 |
| | 2208) | |
| 2209 | CTGATCCCATACAGAAAGTTTCT (SEQ ID NO: | RTT*1, 3, 4 (AG-to-AA PAM silencing edit) |
| | 2209) | |
| 2210 | CCTGATCCCATACAGAAAGCTTCT (SEQ ID NO: | RTT*1, 2, 4 |
| | 2210) | |
| 2211 | CCTGATCCCATACAGAAAGTTTCT (SEQ ID NO: | RTT*1, 3, 4 (AG-to-AA PAM silencing edit) |
| | 2211) | |
| 2212 | GCCTGATCCCATACAGAAAGCTTCT (SEQ ID NO: | RTT*1, 2, 4 |
| | 2212) | |
| 2213 | GCCTGATCCCATACAGAAAGTTTCT (SEQ ID NO: | RTT*1, 3, 4 (AG-to-AA PAM silencing edit) |
| | 2213) | |
| 2214 | AGCCTGATCCCATACAGAAAGCTTCT (SEQ ID | RTT*1, 2, 4 |
| | NO: 2214) | |
| 2215 | AGCCTGATCCCATACAGAAAGTTTCT (SEQ ID | RTT*1, 3, 4 (AG-to-AA PAM silencing edit) |
| | NO: 2215) | |
| 2216 | AAGCCTGATCCCATACAGAAAGCTTCT (SEQ ID | RTT*1, 2, 4 |
| | NO: 2216) | |
| 2217 | AAGCCTGATCCCATACAGAAAGTTTCT (SEQ ID | RTT*1, 3, 4 (AG-to-AA PAM silencing edit) |
| | NO: 2217) | |
| 2218 | GAAGCCTGATCCCATACAGAAAGCTTCT (SEQ ID | RTT*1, 2, 4 |
| | NO: 2218) | |
| 2219 | GAAGCCTGATCCCATACAGAAAGTTTCT (SEQ ID | RTT*1, 3, 4 (AG-to-AA PAM silencing edit) |
| | NO: 2219) | |
| 2220 | GGAAGCCTGATCCCATACAGAAAGCTTCT (SEQ | RTT*1, 2, 4 |
| | ID NO: 2220) | |
| 2221 | GGAAGCCTGATCCCATACAGAAAGTTTCT (SEQ | RTT*1, 3, 4 (AG-to-AA PAM silencing edit) |
| | ID NO: 2221) | |
| 2222 | TGGAAGCCTGATCCCATACAGAAAGCTTCT (SEQ | RTT*1, 2, 4 |
| | ID NO: 2222) | |
| 2223 | TGGAAGCCTGATCCCATACAGAAAGTTTCT (SEQ | RTT*1, 3, 4 (AG-to-AA PAM silencing edit) |
| | ID NO: 2223) | |
| 2224 | GTGGAAGCCTGATCCCATACAGAAAGCTTCT | RTT*1, 2, 4 |
| | (SEQ ID NO: 2224) | |
| 2225 | GTGGAAGCCTGATCCCATACAGAAAGTTTCT | RTT*1, 3, 4 (AG-to-AA PAM silencing edit) |
| | (SEQ ID NO: 2225) | |
| 2226 | AGTGGAAGCCTGATCCCATACAGAAAGCTTCT | RTT*1, 2, 4 |
| | (SEQ ID NO: 2226) | |
| 2227 | AGTGGAAGCCTGATCCCATACAGAAAGTTTCT | RTT*1, 3, 4 (AG-to-AA PAM silencing edit) |
| | (SEQ ID NO: 2227) | |
| 2228 | AAGTGGAAGCCTGATCCCATACAGAAAGCTTCT | RTT*1, 2, 4 |
| | (SEQ ID NO: 2228) | |
| 2229 | AAGTGGAAGCCTGATCCCATACAGAAAGTTTCT | RTT*1, 3, 4 (AG-to-AA PAM silencing edit) |
| | (SEQ ID NO: 2229) | |
| 2230 | AAAGTGGAAGCCTGATCCCATACAGAAAGCTTC | RTT*1, 2, 4 |
| | T (SEQ ID NO: 2230) | |
| 2231 | AAAGTGGAAGCCTGATCCCATACAGAAAGTTTCT | RTT*1, 3, 4 (AG-to-AA PAM silencing edit) |
| | (SEQ ID NO: 2231) | |
| 2232 | CAAAGTGGAAGCCTGATCCCATACAGAAAGCTT | RTT*1, 2, 4 |
| | CT (SEQ ID NO: 2232) | |
| 2233 | CAAAGTGGAAGCCTGATCCCATACAGAAAGTTT | RTT*1, 3, 4 (AG-to-AA PAM silencing edit) |
| | CT (SEQ ID NO: 2233) | |
| 2234 | CCAAAGTGGAAGCCTGATCCCATACAGAAAGCT | RTT*1, 2, 4 |
| | TCT (SEQ ID NO: 2234) | |
| 2235 | CCAAAGTGGAAGCCTGATCCCATACAGAAAGTT | RTT*1, 3, 4 (AG-to-AA PAM silencing edit) |
| | TCT (SEQ ID NO: 2235) | |
| 2236 | TCCAAAGTGGAAGCCTGATCCCATACAGAAAGC | RTT*1, 2, 4 |
| | TTCT (SEQ ID NO: 2236) | |
| 2237 | TCCAAAGTGGAAGCCTGATCCCATACAGAAAGT | RTT*1, 3, 4 (AG-to-AA PAM silencing edit) |
| | TTCT (SEQ ID NO: 2237) | |
| 2238 | TTCCAAAGTGGAAGCCTGATCCCATACAGAAAG | RTT*1, 2, 4 |
| | CTTCT (SEQ ID NO: 2238) | |
| 2239 | TTCCAAAGTGGAAGCCTGATCCCATACAGAAAG | RTT*1, 3, 4 (AG-to-AA PAM silencing edit) |
| | TTTCT (SEQ ID NO: 2239) | |
| 2240 | TTTCCAAAGTGGAAGCCTGATCCCATACAGAAA | RTT*1, 2, 4 |
| | GCTTCT (SEQ ID NO: 2240) | |
| 2241 | TTTCCAAAGTGGAAGCCTGATCCCATACAGAAA | RTT*1, 3, 4 (AG-to-AA PAM silencing edit) |
| | GTTTCT (SEQ ID NO: 2241) | |
| 2242 | GTTTCCAAAGTGGAAGCCTGATCCCATACAGAA | RTT*1, 2, 4 |
| | AGCTTCT (SEQ ID NO: 2242) | |
| 2243 | GTTTCCAAAGTGGAAGCCTGATCCCATACAGAA | RTT*1, 3, 4 (AG-to-AA PAM silencing edit) |
| | AGTTTCT (SEQ ID NO: 2243) | |
| 4 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b*1 ngRNA spacer (AG PAM) |
| 195 | ACATCTTCCTTAGGAACTCC (SEQ ID NO: 195) | PE3 ngRNA spacer (TG PAM) |
| 196 | ACTACTTACCATCTCTTTCA (SEQ ID NO: 196) | PE3 ngRNA spacer (AG PAM) |
| 197 | ACTCCTGGAACTGAGCCACG (SEQ ID NO: 197) | PE3 ngRNA spacer (TG PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 383 | GATCCCATACAGAAAGCTTC (SEQ ID NO: 383) | PE3b*2 ngRNA spacer (TG PAM) |
| 2244 | GATCCCATACAGAAAGTTTC (SEQ ID NO: 2244) | PE3b*3 ngRNA spacer (TG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b*4 ngRNA spacer (AG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 30

| Sequence | | |
|---|---|---|
| Number | Sequence | Description |
| 2245 | TTGTGATTATCAGTTTT (SEQ ID NO: 2245) | PEgRNA spacer (GG; GGA PAM) |
| 2246 | CTTGTGATTATCAGTTTT (SEQ ID NO: 2246) | PEgRNA spacer (GG; GGA PAM) |
| 2247 | TCTTGTGATTATCAGTTTT (SEQ ID NO: 2247) | PERNA spacer (GG; GGA PAM) |
| 45 | TTCTTGTGATTATCAGTTTT (SEQ ID NO: 45) | PEgRNA spacer (GG; GGA PAM) |
| 2248 | TTTCTTGTGATTATCAGTTTT (SEQ ID NO: 2248) | PEgRNA spacer (GG; GGA PAM) |
| 2249 | CTTTCTTGTGATTATCAGTTTT (SEQ ID NO: 2249) | PEgRNA spacer (GG; GGA PAM) |
| 2250 | ACTGA | PBS |
| 2251 | ACTGAT | PBS |
| 2252 | ACTGATA | PBS |
| 2253 | ACTGATAA | PBS |
| 2254 | ACTGATAAT | PBS |
| 2255 | ACTGATAATC (SEQ ID NO: 2255) | PBS |
| 2256 | ACTGATAATCA (SEQ ID NO: 2256) | PBS |
| 2257 | ACTGATAATCAC (SEQ ID NO: 2257) | PBS |
| 2258 | ACTGATAATCACA (SEQ ID NO: 2258) | PBS |
| 2259 | ACTGATAATCACAA (SEQ ID NO: 2259) | PBS |
| 2260 | ACTGATAATCACAAG (SEQ ID NO: 2260) | PBS |
| 2261 | ACTGATAATCACAAGA (SEQ ID NO: 2261) | PBS |
| 2262 | ACTGATAATCACAAGAA (SEQ ID NO: 2262) | PBS |
| 2263 | ACTGATAATCACAAGAAA (SEQ ID NO: 2263) | PBS |
| 2264 | ACTGATAATCACAAGAAAG (SEQ ID NO: 2264) | PBS |
| 2265 | CCCATACAGAAAGCTTCTGCATCCAAA (SEQ ID | RTT |
| | NO: 2265) | |
| 2266 | TCCCATACAGAAAGCTTCTGCATCCAAA (SEQ ID | RTT |
| | NO: 2266) | |
| 2267 | ATCCCATACAGAAAGCTTCTGCATCCAAA (SEQ | RTT |
| | ID NO: 2267) | |
| 2268 | GATCCCATACAGAAAGCTTCTGCATCCAAA (SEQ | RTT |
| | ID NO: 2268) | |
| 2269 | TGATCCCATACAGAAAGCTTCTGCATCCAAA | RTT |
| | (SEQ ID NO: 2269) | |
| 2270 | CTGATCCCATACAGAAAGCTTCTGCATCCAAA | RTT |
| | (SEQ ID NO: 2270) | |
| 2271 | CCTGATCCCATACAGAAAGCTTCTGCATCCAAA | RTT |
| | (SEQ ID NO: 2271) | |
| 2272 | GCCTGATCCCATACAGAAAGCTTCTGCATCCAAA | RTT |
| | (SEQ ID NO: 2272) | |
| 2273 | AGCCTGATCCCATACAGAAAGCTTCTGCATCCAA | RTT |
| | A (SEQ ID NO: 2273) | |
| 2274 | AAGCCTGATCCCATACAGAAAGCTTCTGCATCCA | RTT |
| | AA (SEQ ID NO: 2274) | |
| 2275 | GAAGCCTGATCCCATACAGAAAGCTTCTGCATCC | RTT |
| | AAA (SEQ ID NO: 2275) | |
| 2276 | GGAAGCCTGATCCCATACAGAAAGCTTCTGCATC | RTT |
| | CAAA (SEQ ID NO: 2276) | |
| 2277 | TGGAAGCCTGATCCCATACAGAAAGCTTCTGCAT | RTT |
| | CCAAA (SEQ ID NO: 2277) | |
| 2278 | GTGGAAGCCTGATCCCATACAGAAAGCTTCTGC | RTT |
| | ATCCAAA (SEQ ID NO: 2278) | |
| 380 | AAAGCTTCTGCATCCAAAAC (SEQ ID NO: 380) | PE3 ngRNA spacer (TG; TGA PAM) |
| 4 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b ngRNA spacer (AG PAM) |
| 195 | ACATCTTCCTTAGGAACTCC (SEQ ID NO: 195) | PE3 ngRNA spacer (TG PAM) |
| 196 | ACTACTTACCATCTCTTTCA (SEQ ID NO: 196) | PE3 ngRNA spacer (AG PAM) |
| 197 | ACTCCTGGAACTGAGCCACG (SEQ ID NO: 197) | PE3 ngRNA spacer (TG; TGA PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG; GGA PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG; AGA PAM) |
| 203 | CGTGAAGACAGGTGTCTTGC (SEQ ID NO: 203) | PE3 ngRNA spacer (TG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 383 | GATCCCATACAGAAAGCTTC (SEQ ID NO: 383) | PE3b ngRNA spacer (TG PAM) |
| 210 | GGCTTCATACATCTTCCTTA (SEQ ID NO: 210) | PE3 ngRNA spacer (GG; GGA PAM) |
| 211 | GGGTTTCCAAAGTGGAAGCC (SEQ ID NO: 211) | PE3 ngRNA spacer (TG; TGA PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b ngRNA spacer (AG; AGA PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG; TGA PAM) |
| 216 | TCTTGCTGGGTTTCCAAAGT (SEQ ID NO: 216) | PE3 ngRNA spacer (GG; GGA PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 31

| Sequence | | |
|---|---|---|
| Number | Sequence | Description |
| 2279 | CTGGGTTTCCAAAGTGG (SEQ ID NO: 2279) | PEgRNA spacer (AAG PAM) |
| 2280 | GCTGGGTTTCCAAAGTGG (SEQ ID NO: 2280) | PEgRNA spacer (AAG PAM) |
| 2281 | TGCTGGGTTTCCAAAGTGG (SEQ ID NO: 2281) | PEgRNA spacer (AAG PAM) |
| 488 | TTGCTGGGTTTCCAAAGTGG (SEQ ID NO: 488) | PEgRNA spacer (AAG PAM) |
| 2282 | CTTGCTGGGTTTCCAAAGTGG (SEQ ID NO: 2282) | PEgRNA spacer (AAG PAM) |
| 2283 | TCTTGCTGGGTTTCCAAAGTGG (SEQ ID NO: 2283) | PEgRNA spacer (AAG PAM) |
| 2284 | CTTTG | PBS |
| 2285 | CTTTGG | PBS |
| 2286 | CTTTGGA | PBS |
| 2287 | CTTTGGAA | PBS |
| 2288 | CTTTGGAAA | PBS |
| 2289 | CTTTGGAAAC (SEQ ID NO: 2289) | PBS |
| 2290 | CTTTGGAAACC (SEQ ID NO: 2290) | PBS |
| 2291 | CTTTGGAAACCC (SEQ ID NO: 2291) | PBS |
| 2292 | CTTTGGAAACCCA (SEQ ID NO: 2292) | PBS |
| 2293 | CTTTGGAAACCCAG (SEQ ID NO: 2293) | PBS |
| 2294 | CTTTGGAAACCCAGC (SEQ ID NO: 2294) | PBS |
| 2295 | CTTTGGAAACCCAGCA (SEQ ID NO: 2295) | PBS |
| 2296 | CTTTGGAAACCCAGCAA (SEQ ID NO: 2296) | PBS |
| 2297 | CTTTGGAAACCCAGCAAG (SEQ ID NO: 2297) | PBS |
| 2298 | CTTTGGAAACCCAGCAAGA (SEQ ID NO: 2298) | PBS |
| 2299 | GATCAGGCATCCA (SEQ ID NO: 2299) | RTT (AAG-to-ATG PAM silencing edit) |
| 2300 | GATCAGGCCTCCA (SEQ ID NO: 2300) | RTT (AAG-to-AGG PAM silencing edit) |
| 2301 | GATCAGGCGTCCA (SEQ ID NO: 2301) | RTT (AAG-to-ACG PAM silencing edit) |
| 2302 | GATCAGGCTTCCA (SEQ ID NO: 2302) | RTT |
| 2303 | GGATCAGGCATCCA (SEQ ID NO: 2303) | RTT (AAG-to-ATG PAM silencing edit) |
| 2304 | GGATCAGGCCTCCA (SEQ ID NO: 2304) | RTT (AAG-to-AGG PAM silencing edit) |
| 2305 | GGATCAGGCGTCCA (SEQ ID NO: 2305) | RTT (AAG-to-ACG PAM silencing edit) |
| 2306 | GGATCAGGCTTCCA (SEQ ID NO: 2306) | RTT |
| 2307 | GGGATCAGGCATCCA (SEQ ID NO: 2307) | RTT (AAG-to-ATG PAM silencing edit) |
| 2308 | GGGATCAGGCCTCCA (SEQ ID NO: 2308) | RTT (AAG-to-AGG PAM silencing edit) |
| 2309 | GGGATCAGGCGTCCA (SEQ ID NO: 2309) | RTT (AAG-to-ACG PAM silencing edit) |
| 2310 | GGGATCAGGCTTCCA (SEQ ID NO: 2310) | RTT |
| 2311 | TGGGATCAGGCATCCA (SEQ ID NO: 2311) | RTT (AAG-to-ATG PAM silencing edit) |
| 2312 | TGGGATCAGGCCTCCA (SEQ ID NO: 2312) | RTT (AAG-to-AGG PAM silencing edit) |
| 2313 | TGGGATCAGGCGTCCA (SEQ ID NO: 2313) | RTT (AAG-to-ACG PAM silencing edit) |
| 2314 | TGGGATCAGGCTTCCA (SEQ ID NO: 2314) | RTT |
| 2315 | ATGGGATCAGGCATCCA (SEQ ID NO: 2315) | RTT (AAG-to-ATG PAM silencing edit) |
| 2316 | ATGGGATCAGGCCTCCA (SEQ ID NO: 2316) | RTT (AAG-to-AGG PAM silencing edit) |
| 2317 | ATGGGATCAGGCGTCCA (SEQ ID NO: 2317) | RTT (AAG-to-ACG PAM silencing edit) |
| 2318 | ATGGGATCAGGCTTCCA (SEQ ID NO: 2318) | RTT |
| 2319 | TATGGGATCAGGCATCCA (SEQ ID NO: 2319) | RTT (AAG-to-ATG PAM silencing edit) |
| 2320 | TATGGGATCAGGCCTCCA (SEQ ID NO: 2320) | RTT (AAG-to-AGG PAM silencing edit) |
| 2321 | TATGGGATCAGGCGTCCA (SEQ ID NO: 2321) | RTT (AAG-to-ACG PAM silencing edit) |
| 2322 | TATGGGATCAGGCTTCCA (SEQ ID NO: 2322) | RTT |
| 2323 | GTATGGGATCAGGCATCCA (SEQ ID NO: 2323) | RTT (AAG-to-ATG PAM silencing edit) |
| 2324 | GTATGGGATCAGGCCTCCA (SEQ ID NO: 2324) | RTT (AAG-to-AGG PAM silencing edit) |
| 2325 | GTATGGGATCAGGCGTCCA (SEQ ID NO: 2325) | RTT (AAG-to-ACG PAM silencing edit) |
| 2326 | GTATGGGATCAGGCTTCCA (SEQ ID NO: 2326) | RTT |
| 2327 | TGTATGGGATCAGGCATCCA (SEQ ID NO: 2327) | RTT (AAG-to-ATG PAM silencing edit) |
| 2328 | TGTATGGGATCAGGCCTCCA (SEQ ID NO: 2328) | RTT (AAG-to-AGG PAM silencing edit) |
| 2329 | TGTATGGGATCAGGCGTCCA (SEQ ID NO: 2329) | RTT (AAG-to-ACG PAM silencing edit) |
| 2330 | TGTATGGGATCAGGCTTCCA (SEQ ID NO: 2330) | RTT |
| 2331 | CTGTATGGGATCAGGCATCCA (SEQ ID NO: 2331) | RTT (AAG-to-ATG PAM silencing edit) |
| 2332 | CTGTATGGGATCAGGCCTCCA (SEQ ID NO: 2332) | RTT (AAG-to-AGG PAM silencing edit) |
| 2333 | CTGTATGGGATCAGGCGTCCA (SEQ ID NO: 2333) | RTT (AAG-to-ACG PAM silencing edit) |
| 2334 | CTGTATGGGATCAGGCTTCCA (SEQ ID NO: 2334) | RTT |
| 2335 | TCTGTATGGGATCAGGCATCCA (SEQ ID NO: 2335) | RTT (AAG-to-ATG PAM silencing edit) |
| 2336 | TCTGTATGGGATCAGGCCTCCA (SEQ ID NO: 2336) | RTT (AAG-to-AGG PAM silencing edit) |
| 2337 | TCTGTATGGGATCAGGCGTCCA (SEQ ID NO: 2337) | RTT (AAG-to-ACG PAM silencing edit) |
| 2338 | TCTGTATGGGATCAGGCTTCCA (SEQ ID NO: 2338) | RTT |
| 2339 | TTCTGTATGGGATCAGGCATCCA (SEQ ID NO: | RTT (AAG-to-ATG PAM silencing edit) |
| | 2339) | |
| 2340 | TTCTGTATGGGATCAGGCCTCCA (SEQ ID NO: | RTT (AAG-to-AGG PAM silencing edit) |
| | 2340) | |
| 2341 | TTCTGTATGGGATCAGGCGTCCA (SEQ ID NO: | RTT (AAG-to-ACG PAM silencing edit) |
| | 2341) | |
| 2342 | TTCTGTATGGGATCAGGCTTCCA (SEQ ID NO: | RTT |
| | 2342) | |
| 2343 | TTTCTGTATGGGATCAGGCATCCA (SEQ ID NO: | RTT (AAG-to-ATG PAM silencing edit) |
| | 2343) | |
| 2344 | TTTCTGTATGGGATCAGGCCTCCA (SEQ ID NO: | RTT (AAG-to-AGG PAM silencing edit) |
| | 2344) | |
| 2345 | TTTCTGTATGGGATCAGGCGTCCA (SEQ ID NO: | RTT (AAG-to-ACG PAM silencing edit) |
| | 2345) | |
| 2346 | TTTCTGTATGGGATCAGGCTTCCA (SEQ ID NO: | RTT |
| | 2346) | |
| 2347 | CTTTCTGTATGGGATCAGGCATCCA (SEQ ID NO: | RTT (AAG-to-ATG PAM silencing edit) |
| | 2347) | |
| 2348 | CTTTCTGTATGGGATCAGGCCTCCA (SEQ ID NO: | RTT (AAG-to-AGG PAM silencing edit) |
| | 2348) | |
| 2349 | CTTTCTGTATGGGATCAGGCGTCCA (SEQ ID NO: | RTT (AAG-to-ACG PAM silencing edit) |
| | 2349) | |
| 2350 | CTTTCTGTATGGGATCAGGCTTCCA (SEQ ID NO: | RTT |
| | 2350) | |
| 2351 | GCTTTCTGTATGGGATCAGGCATCCA (SEQ ID | RTT (AAG-to-ATG PAM silencing edit) |
| | NO: 2351) | |
| 2352 | GCTTTCTGTATGGGATCAGGCCTCCA (SEQ ID NO: | RTT (AAG-to-AGG PAM silencing edit) |
| | 2352) | |
| 2353 | GCTTTCTGTATGGGATCAGGCGTCCA (SEQ ID | RTT (AAG-to-ACG PAM silencing edit) |
| | NO: 2353) | |
| 2354 | GCTTTCTGTATGGGATCAGGCTTCCA (SEQ ID NO: | RTT |
| | 2354) | |
| 2355 | AGCTTTCTGTATGGGATCAGGCATCCA (SEQ ID | RTT (AAG-to-ATG PAM silencing edit) |
| | NO: 2355) | |
| 2356 | AGCTTTCTGTATGGGATCAGGCCTCCA (SEQ ID | RTT (AAG-to-AGG PAM silencing edit) |
| | NO: 2356) | |
| 2357 | AGCTTTCTGTATGGGATCAGGCGTCCA (SEQ ID | RTT (AAG-to-ACG PAM silencing edit) |
| | NO: 2357) | |
| 2358 | AGCTTTCTGTATGGGATCAGGCTTCCA (SEQ ID | RTT |
| | NO: 2358) | |
| 2359 | AAGCTTTCTGTATGGGATCAGGCATCCA (SEQ ID | RTT (AAG-to-ATG PAM silencing edit) |
| | NO: 2359) | |
| 2360 | AAGCTTTCTGTATGGGATCAGGCCTCCA (SEQ ID | RTT (AAG-to-AGG PAM silencing edit) |
| | NO: 2360) | |
| 2361 | AAGCTTTCTGTATGGGATCAGGCGTCCA (SEQ ID | RTT (AAG-to-ACG PAM silencing edit) |
| | NO: 2361) | |
| 2362 | AAGCTTTCTGTATGGGATCAGGCTTCCA (SEQ ID | RTT |
| | NO: 2362) | |
| 2363 | GAAGCTTTCTGTATGGGATCAGGCATCCA (SEQ | RTT (AAG-to-ATG PAM silencing edit) |
| | ID NO: 2363) | |
| 2364 | GAAGCTTTCTGTATGGGATCAGGCCTCCA (SEQ | RTT (AAG-to-AGG PAM silencing edit) |
| | ID NO: 2364) | |
| 2365 | GAAGCTTTCTGTATGGGATCAGGCGTCCA (SEQ | RTT (AAG-to-ACG PAM silencing edit) |
| | ID NO: 2365) | |
| 2366 | GAAGCTTTCTGTATGGGATCAGGCTTCCA (SEQ | RTT |
| | ID NO: 2366) | |
| 2367 | AGAAGCTTTCTGTATGGGATCAGGCATCCA (SEQ | RTT (AAG-to-ATG PAM silencing edit) |
| | ID NO: 2367) | |
| 2368 | AGAAGCTTTCTGTATGGGATCAGGCCTCCA (SEQ | RTT (AAG-to-AGG PAM silencing edit) |
| | ID NO: 2368) | |
| 2369 | AGAAGCTTTCTGTATGGGATCAGGCGTCCA (SEQ | RTT (AAG-to-ACG PAM silencing edit) |
| | ID NO: 2369) | |
| 2370 | AGAAGCTTTCTGTATGGGATCAGGCTTCCA (SEQ | RTT |
| | ID NO: 2370) | |
| 2371 | CAGAAGCTTTCTGTATGGGATCAGGCATCCA | RTT (AAG-to-ATG PAM silencing edit) |
| | (SEQ ID NO: 2371) | |
| 2372 | CAGAAGCTTTCTGTATGGGATCAGGCCTCCA | RTT (AAG-to-AGG PAM silencing edit) |
| | (SEQ ID NO: 2372) | |
| 2373 | CAGAAGCTTTCTGTATGGGATCAGGCGTCCA | RTT (AAG-to-ACG PAM silencing edit) |
| | (SEQ ID NO: 2373) | |
| 2374 | CAGAAGCTTTCTGTATGGGATCAGGCTTCCA | RTT |
| | (SEQ ID NO: 2374) | |
| 2375 | GCAGAAGCTTTCTGTATGGGATCAGGCATCCA | RTT (AAG-to-ATG PAM silencing edit) |
| | (SEQ ID NO: 2375) | |
| 2376 | GCAGAAGCTTTCTGTATGGGATCAGGCCTCCA | RTT (AAG-to-AGG PAM silencing edit) |
| | (SEQ ID NO: 2376) | |
| 2377 | GCAGAAGCTTTCTGTATGGGATCAGGCGTCCA | RTT (AAG-to-ACG PAM silencing edit) |
| | (SEQ ID NO: 2377) | |
| 2378 | GCAGAAGCTTTCTGTATGGGATCAGGCTTCCA | RTT |
| | (SEQ ID NO: 2378) | |
| 2379 | TGCAGAAGCTTTCTGTATGGGATCAGGCATCCA | RTT (AAG-to-ATG PAM silencing edit) |
| | (SEQ ID NO: 2379) | |
| 2380 | TGCAGAAGCTTTCTGTATGGGATCAGGCCTCCA | RTT (AAG-to-AGG PAM silencing edit) |
| | (SEQ ID NO: 2380) | |
| 2381 | TGCAGAAGCTTTCTGTATGGGATCAGGCGTCCA | RTT (AAG-to-ACG PAM silencing edit) |
| | (SEQ ID NO: 2381) | |
| 2382 | TGCAGAAGCTTTCTGTATGGGATCAGGCTTCCA | RTT |
| | (SEQ ID NO: 2382) | |
| 2383 | ATGCAGAAGCTTTCTGTATGGGATCAGGCATCCA | RTT (AAG-to-ATG PAM silencing edit) |
| | (SEQ ID NO: 2383) | |
| 2384 | ATGCAGAAGCTTTCTGTATGGGATCAGGCCTCCA | RTT (AAG-to-AGG PAM silencing edit) |
| | (SEQ ID NO: 2384) | |
| 2385 | ATGCAGAAGCTTTCTGTATGGGATCAGGCGTCCA | RTT (AAG-to-ACG PAM silencing edit) |
| | (SEQ ID NO: 2385) | |
| 2386 | ATGCAGAAGCTTTCTGTATGGGATCAGGCTTCCA | RTT |
| | (SEQ ID NO: 2386) | |
| 2387 | GATGCAGAAGCTTTCTGTATGGGATCAGGCATCC | RTT (AAG-to-ATG PAM silencing edit) |
| | A (SEQ ID NO: 2387) | |
| 2388 | GATGCAGAAGCTTTCTGTATGGGATCAGGCCTCC | RTT (AAG-to-AGG PAM silencing edit) |
| | A (SEQ ID NO: 2388) | |
| 2389 | GATGCAGAAGCTTTCTGTATGGGATCAGGCGTCC | RTT (AAG-to-ACG PAM silencing edit) |
| | A (SEQ ID NO: 2389) | |
| 2390 | GATGCAGAAGCTTTCTGTATGGGATCAGGCTTCC | RTT |
| | A (SEQ ID NO: 2390) | |
| 2391 | GGATGCAGAAGCTTTCTGTATGGGATCAGGCATC | RTT (AAG-to-ATG PAM silencing edit) |
| | CA (SEQ ID NO: 2391) | |
| 2392 | GGATGCAGAAGCTTTCTGTATGGGATCAGGCCTC | RTT (AAG-to-AGG PAM silencing edit) |
| | CA (SEQ ID NO: 2392) | |
| 2393 | GGATGCAGAAGCTTTCTGTATGGGATCAGGCGTC | RTT (AAG-to-ACG PAM silencing edit) |
| | CA (SEQ ID NO: 2393) | |
| 2394 | GGATGCAGAAGCTTTCTGTATGGGATCAGGCTTC | RTT |
| | CA (SEQ ID NO: 2394) | |
| 2395 | TGGATGCAGAAGCTTTCTGTATGGGATCAGGCAT | RTT (AAG-to-ATG PAM silencing edit) |
| | CCA (SEQ ID NO: 2395) | |
| 2396 | TGGATGCAGAAGCTTTCTGTATGGGATCAGGCCT | RTT (AAG-to-AGG PAM silencing edit) |
| | CCA (SEQ ID NO: 2396) | |
| 2397 | TGGATGCAGAAGCTTTCTGTATGGGATCAGGCGT | RTT (AAG-to-ACG PAM silencing edit) |
| | CCA (SEQ ID NO: 2397) | |
| 2398 | TGGATGCAGAAGCTTTCTGTATGGGATCAGGCTT | RTT |
| | CCA (SEQ ID NO: 2398) | |
| 2399 | TTGGATGCAGAAGCTTTCTGTATGGGATCAGGCA | RTT (AAG-to-ATG PAM silencing edit) |
| | TCCA (SEQ ID NO: 2399) | |
| 2400 | TTGGATGCAGAAGCTTTCTGTATGGGATCAGGCC | RTT (AAG-to-AGG PAM silencing edit) |
| | TCCA (SEQ ID NO: 2400) | |
| 2401 | TTGGATGCAGAAGCTTTCTGTATGGGATCAGGCG | RTT (AAG-to-ACG PAM silencing edit) |
| | TCCA (SEQ ID NO: 2401) | |
| 2402 | TTGGATGCAGAAGCTTTCTGTATGGGATCAGGCT | RTT |
| | TCCA (SEQ ID NO: 2402) | |
| 2403 | TTTGGATGCAGAAGCTTTCTGTATGGGATCAGGC | RTT (AAG-to-ATG PAM silencing edit) |
| | ATCCA (SEQ ID NO: 2403) | |
| 2404 | TTTGGATGCAGAAGCTTTCTGTATGGGATCAGGC | RTT (AAG-to-AGG PAM silencing edit) |
| | CTCCA (SEQ ID NO: 2404) | |
| 2405 | TTTGGATGCAGAAGCTTTCTGTATGGGATCAGGC | RTT (AAG-to-ACG PAM silencing edit) |
| | GTCCA (SEQ ID NO: 2405) | |
| 2406 | TTTGGATGCAGAAGCTTTCTGTATGGGATCAGGC | RTT |
| | TTCCA (SEQ ID NO: 2406) | |
| 2407 | TTTTGGATGCAGAAGCTTTCTGTATGGGATCAGG | RTT (AAG-to-ATG PAM silencing edit) |
| | CATCCA (SEQ ID NO: 2407) | |
| 2408 | TTTTGGATGCAGAAGCTTTCTGTATGGGATCAGG | RTT (AAG-to-AGG PAM silencing edit) |
| | CCTCCA (SEQ ID NO: 2408) | |
| 2409 | TTTTGGATGCAGAAGCTTTCTGTATGGGATCAGG | RTT (AAG-to-ACG PAM silencing edit) |
| | CGTCCA (SEQ ID NO: 2409) | |
| 2410 | TTTTGGATGCAGAAGCTTTCTGTATGGGATCAGG | RTT |
| | CTTCCA (SEQ ID NO: 2410) | |
| 127 | AGAAGCTTTCTGTATGGGAT (SEQ ID NO: 127) | PE3b ngRNA spacer (CAG PAM) |
| 1223 | ATCACCTTTTCGCTTTTTCC (SEQ ID NO: 1223) | PE3 ngRNA spacer (AAG PAM) |
| 418 | ATTATCAGTTTTGGATGCAG (SEQ ID NO: 418) | PE3 ngRNA spacer (AAG PAM) |
| 1224 | CAAGATGGCTCAAGATTCAG (SEQ ID NO: 1224) | PE3 ngRNA spacer (TAG PAM) |
| 1225 | CGCTTTTTCCAAGATGGCTC (SEQ ID NO: 1225) | PE3 ngRNA spacer (AAG PAM) |
| 1226 | GTGATTATCAGTTTTGGATG (SEQ ID NO: 1226) | PE3 ngRNA spacer (CAG PAM) |
| 1227 | GTGCTGGACAGGCTGCTGTG (SEQ ID NO: 1227) | PE3 ngRNA spacer (AAG PAM) |
| 1228 | TAGATCTTTCTTGTGATTAT (SEQ ID NO: 1228) | PE3 ngRNA spacer (CAG PAM) |
| 1229 | TTCCAAGATGGCTCAAGATT (SEQ ID NO: 1229) | PE3 ngRNA spacer (CAG PAM) |

TABLE 32

| Sequence | | |
|---|---|---|
| Number | Sequence | Description |
| 2411 | TGATTATCAGTTTTGGA (SEQ ID NO: 2411) | PEgRNA spacer (TG PAM) |
| 2412 | GTGATTATCAGTTTTGGA (SEQ ID NO: 2412) | PEgRNA spacer (TG PAM) |
| 2413 | TGTGATTATCAGTTTTGGA (SEQ ID NO: 2413) | PEgRNA spacer (TG PAM) |
| 46 | TTGTGATTATCAGTTTTGGA (SEQ ID NO: 46) | PEgRNA spacer (TG PAM) |
| 2414 | CTTGTGATTATCAGTTTTGGA (SEQ ID NO: 2414) | PEgRNA spacer (TG PAM) |
| 2415 | TCTTGTGATTATCAGTTTTGGA (SEQ ID NO: 2415) | PEgRNA spacer (TG PAM) |
| 2416 | AAAAC | PBS |
| 2417 | AAAACT | PBS |
| 2418 | AAAACTG | PBS |
| 2419 | AAAACTGA | PBS |
| 2420 | AAAACTGAT | PBS |
| 2421 | AAAACTGATA (SEQ ID NO: 2421) | PBS |
| 2422 | AAAACTGATAA (SEQ ID NO: 2422) | PBS |
| 2423 | AAAACTGATAAT (SEQ ID NO: 2423) | PBS |
| 2424 | AAAACTGATAATC (SEQ ID NO: 2424) | PBS |
| 2425 | AAAACTGATAATCA (SEQ ID NO: 2425) | PBS |
| 2426 | AAAACTGATAATCAC (SEQ ID NO: 2426) | PBS |
| 2427 | AAAACTGATAATCACA (SEQ ID NO: 2427) | PBS |
| 2428 | AAAACTGATAATCACAA (SEQ ID NO: 2428) | PBS |
| 2429 | AAAACTGATAATCACAAG (SEQ ID NO: 2429) | PBS |
| 2430 | AAAACTGATAATCACAAGA (SEQ ID NO: 2430) | PBS |
| 2431 | CCCATACAGAAAGCTTCTGCATCC (SEQ ID NO: | RTT |
| | 2431) | |
| 2432 | TCCCATACAGAAAGCTTCTGCATCC (SEQ ID NO: | RTT |
| | 2432) | |
| 2433 | ATCCCATACAGAAAGCTTCTGCATCC (SEQ ID | RTT |
| | NO: 2433) | |
| 2434 | GATCCCATACAGAAAGCTTCTGCATCC (SEQ ID | RTT |
| | NO: 2434) | |
| 2435 | TGATCCCATACAGAAAGCTTCTGCATCC (SEQ ID | RTT |
| | NO: 2435) | |
| 2436 | CTGATCCCATACAGAAAGCTTCTGCATCC (SEQ | RTT |
| | ID NO: 2436) | |
| 2437 | CCTGATCCCATACAGAAAGCTTCTGCATCC (SEQ | RTT |
| | ID NO: 2437) | |
| 2438 | GCCTGATCCCATACAGAAAGCTTCTGCATCC | RTT |
| | (SEQ ID NO: 2438) | |
| 2439 | AGCCTGATCCCATACAGAAAGCTTCTGCATCC | RTT |
| | (SEQ ID NO: 2439) | |
| 2440 | AAGCCTGATCCCATACAGAAAGCTTCTGCATCC | RTT |
| | (SEQ ID NO: 2440) | |
| 2441 | GAAGCCTGATCCCATACAGAAAGCTTCTGCATCC | RTT |
| | (SEQ ID NO: 2441) | |
| 2442 | GGAAGCCTGATCCCATACAGAAAGCTTCTGCATC | RTT |
| | C (SEQ ID NO: 2442) | |
| 2443 | TGGAAGCCTGATCCCATACAGAAAGCTTCTGCAT | RTT |
| | CC (SEQ ID NO: 2443) | |
| 2444 | GTGGAAGCCTGATCCCATACAGAAAGCTTCTGC | RTT |
| | ATCC (SEQ ID NO: 2444) | |
| 2445 | AGTGGAAGCCTGATCCCATACAGAAAGCTTCTG | RTT |
| | CATCC (SEQ ID NO: 2445) | |
| 2446 | AAGTGGAAGCCTGATCCCATACAGAAAGCTTCT | RTT |
| | GCATCC (SEQ ID NO: 2446) | |
| 2447 | AAAGTGGAAGCCTGATCCCATACAGAAAGCTTC | RTT |
| | TGCATCC (SEQ ID NO: 2447) | |
| 4 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b ngRNA spacer (AG PAM) |
| 195 | ACATCTTCCTTAGGAACTCC (SEQ ID NO: 195) | PE3 ngRNA spacer (TG PAM) |
| 196 | ACTACTTACCATCTCTTTCA (SEQ ID NO: 196) | PE3 ngRNA spacer (AG PAM) |
| 197 | ACTCCTGGAACTGAGCCACG (SEQ ID NO: 197) | PE3 ngRNA spacer (TG PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 383 | GATCCCATACAGAAAGCTTC (SEQ ID NO: 383) | PE3b ngRNA spacer (TG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b ngRNA spacer (AG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 33

| Sequence | | |
|---|---|---|
| Number | Sequence | Description |
| 2448 | CTTGTGATTATCAGTTT (SEQ ID NO: 2448) | PEgRNA spacer (TG; TGG PAM) |
| 2449 | TCTTGTGATTATCAGTTT (SEQ ID NO: 2449) | PEgRNA spacer (TG; TGG PAM) |
| 2450 | TTCTTGTGATTATCAGTTT (SEQ ID NO: 2450) | PEgRNA spacer (TG; TGG PAM) |
| 47 | TTTCTTGTGATTATCAGTTT (SEQ ID NO: 47) | PEgRNA spacer (TG; TGG PAM) |
| 2451 | CTTTCTTGTGATTATCAGTTT (SEQ ID NO: 2451) | PERNA spacer (TG; TGG PAM) |
| 2452 | TCTTTCTTGTGATTATCAGTTT (SEQ ID NO: 2452) | PEgRNA spacer (TG; TGG PAM) |
| 2453 | CTGAT | PBS |
| 2454 | CTGATA | PBS |
| 2455 | CTGATAA | PBS |
| 2456 | CTGATAAT | PBS |
| 2457 | CTGATAATC | PBS |
| 2458 | CTGATAATCA (SEQ ID NO: 2458) | PBS |
| 2459 | CTGATAATCAC (SEQ ID NO: 2459) | PBS |
| 2460 | CTGATAATCACA (SEQ ID NO: 2460) | PBS |
| 2461 | CTGATAATCACAA (SEQ ID NO: 2461) | PBS |
| 2462 | CTGATAATCACAAG (SEQ ID NO: 2462) | PBS |
| 2463 | CTGATAATCACAAGA (SEQ ID NO: 2463) | PBS |
| 2464 | CTGATAATCACAAGAA (SEQ ID NO: 2464) | PBS |
| 2465 | CTGATAATCACAAGAAA (SEQ ID NO: 2465) | PBS |
| 2466 | CTGATAATCACAAGAAAG (SEQ ID NO: 2466) | PBS |
| 2467 | CTGATAATCACAAGAAAGA (SEQ ID NO: 2467) | PBS |
| 2468 | CCCATACAGAAAGCTTCTGCATCCAAAA (SEQ ID | RTT |
| | NO: 2468) | |
| 2469 | TCCCATACAGAAAGCTTCTGCATCCAAAA (SEQ | RTT |
| | ID NO: 2469) | |
| 2470 | ATCCCATACAGAAAGCTTCTGCATCCAAAA (SEQ | RTT |
| | ID NO: 2470) | |
| 2471 | GATCCCATACAGAAAGCTTCTGCATCCAAAA | RTT |
| | (SEQ ID NO: 2471) | |
| 2472 | TGATCCCATACAGAAAGCTTCTGCATCCAAAA | RTT |
| | (SEQ ID NO: 2472) | |
| 2473 | CTGATCCCATACAGAAAGCTTCTGCATCCAAAA | RTT |
| | (SEQ ID NO: 2473) | |
| 2474 | CCTGATCCCATACAGAAAGCTTCTGCATCCAAAA | RTT |
| | (SEQ ID NO: 2474) | |
| 2475 | GCCTGATCCCATACAGAAAGCTTCTGCATCCAAA | RTT |
| | A (SEQ ID NO: 2475) | |
| 2476 | AGCCTGATCCCATACAGAAAGCTTCTGCATCCAA | RTT |
| | AA (SEQ ID NO: 2476) | |
| 2477 | AAGCCTGATCCCATACAGAAAGCTTCTGCATCCA | RTT |
| | AAA (SEQ ID NO: 2477) | |
| 2478 | GAAGCCTGATCCCATACAGAAAGCTTCTGCATCC | RTT |
| | AAAA (SEQ ID NO: 2478) | |
| 2479 | GGAAGCCTGATCCCATACAGAAAGCTTCTGCATC | RTT |
| | CAAAA (SEQ ID NO: 2479) | |
| 2480 | TGGAAGCCTGATCCCATACAGAAAGCTTCTGCAT | RTT |
| | CCAAAA (SEQ ID NO: 2480) | |
| 380 | AAAGCTTCTGCATCCAAAAC (SEQ ID NO: 380) | PE3 ngRNA spacer (TG PAM) |
| 4 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b ngRNA spacer (AG PAM) |
| 195 | ACATCTTCCTTAGGAACTCC (SEQ ID NO: 195) | PE3 ngRNA spacer (TG; TGG PAM) |
| 196 | ACTACTTACCATCTCTTTCA (SEQ ID NO: 196) | PE3 ngRNA spacer (AG; AGG PAM) |
| 197 | ACTCCTGGAACTGAGCCACG (SEQ ID NO: 197) | PE3 ngRNA spacer (TG PAM) |
| 199 | AGGCTTCATACATCTTCCTT (SEQ ID NO: 199) | PE3 ngRNA spacer (AG; AGG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG; TGG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG; AGG PAM) |
| 383 | GATCCCATACAGAAAGCTTC (SEQ ID NO: 383) | PE3b ngRNA spacer (TG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG; TGG PAM) |
| 213 | GTGAAGACAGGTGTCTTGCT (SEQ ID NO: 213) | PE3 ngRNA spacer (GG; GGG PAM) |
| 384 | GTGGAAGCCTGATCCCATAC (SEQ ID NO: 384) | PE3b ngRNA spacer (AG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG; GGG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG; AGG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG; TGG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG; GGG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG; AGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG; TGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG; TGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG; AGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG; AGG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG; TGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG; GGG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338) | |
| 339 | GAACTGAGCCACGTGAAGACGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG; TGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG; TGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG; AGG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 34

| Sequence | | |
|---|---|---|
| Number | Sequence | Description |
| 2481 | TGGATGCAGAAGCTTTC (SEQ ID NO: 2481) | PEgRNA spacer (TG PAM) |
| 2482 | TTGGATGCAGAAGCTTTC (SEQ ID NO: 2482) | PEgRNA spacer (TG PAM) |
| 2483 | TTTGGATGCAGAAGCTTTC (SEQ ID NO: 2483) | PEgRNA spacer (TG PAM) |
| 48 | TTTTGGATGCAGAAGCTTTC (SEQ ID NO: 48) | PERNA spacer (TG PAM) |
| 2484 | GTTTTGGATGCAGAAGCTTTC (SEQ ID NO: 2484) | PERNA spacer (TG PAM) |
| 2485 | AGTTTTGGATGCAGAAGCTTTC (SEQ ID NO: 2485) | PEgRNA spacer (TG PAM) |
| 2486 | AGCTT | PBS |
| 2487 | AGCTTC | PBS |
| 2488 | AGCTTCT | PBS |
| 2489 | AGCTTCTG | PBS |
| 2490 | AGCTTCTGC | PBS |
| 2491 | AGCTTCTGCA (SEQ ID NO: 2491) | PBS |
| 2492 | AGCTTCTGCAT (SEQ ID NO: 2492) | PBS |
| 2493 | AGCTTCTGCATC (SEQ ID NO: 2493) | PBS |
| 2494 | AGCTTCTGCATCC (SEQ ID NO: 2494) | PBS |
| 2495 | AGCTTCTGCATCCA (SEQ ID NO: 2495) | PBS |
| 2496 | AGCTTCTGCATCCAA (SEQ ID NO: 2496) | PBS |
| 2497 | AGCTTCTGCATCCAAA (SEQ ID NO: 2497) | PBS |
| 2498 | AGCTTCTGCATCCAAAA (SEQ ID NO: 2498) | PBS |
| 2499 | AGCTTCTGCATCCAAAAC (SEQ ID NO: 2499) | PBS |
| 2500 | AGCTTCTGCATCCAAAACT (SEQ ID NO: 2500) | PBS |
| 2501 | CCCATACAGAA (SEQ ID NO: 2501) | RTT |
| 2502 | TCCCATACAGAA (SEQ ID NO: 2502) | RTT |
| 2503 | ATCCCATACAGAA (SEQ ID NO: 2503) | RTT |
| 2504 | GATCCCATACAGAA (SEQ ID NO: 2504) | RTT |
| 2505 | TGATCCCATACAGAA (SEQ ID NO: 2505) | RTT |
| 2506 | CTGATCCCATACAGAA (SEQ ID NO: 2506) | RTT |
| 2507 | CCTGATCCCATACAGAA (SEQ ID NO: 2507) | RTT |
| 2508 | GCCTGATCCCATACAGAA (SEQ ID NO: 2508) | RTT |
| 2509 | AGCCTGATCCCATACAGAA (SEQ ID NO: 2509) | RTT |
| 382 | AAGCCTGATCCCATACAGAA (SEQ ID NO: 382) | RTT |
| 2510 | GAAGCCTGATCCCATACAGAA (SEQ ID NO: 2510) | RTT |
| 2511 | GGAAGCCTGATCCCATACAGAA (SEQ ID NO: | RTT |
| | 2511) | |
| 2512 | TGGAAGCCTGATCCCATACAGAA (SEQ ID NO: | RTT |
| | 2512) | |
| 2513 | GTGGAAGCCTGATCCCATACAGAA (SEQ ID NO: | RTT |
| | 2513) | |
| 2514 | AGTGGAAGCCTGATCCCATACAGAA (SEQ ID NO: | RTT |
| | 2514) | |
| 2515 | AAGTGGAAGCCTGATCCCATACAGAA (SEQ ID | RTT |
| | NO: 2515) | |
| 2516 | AAAGTGGAAGCCTGATCCCATACAGAA (SEQ ID | RTT |
| | NO: 2516) | |
| 2517 | CAAAGTGGAAGCCTGATCCCATACAGAA (SEQ ID | RTT |
| | NO: 2517) | |
| 2518 | CCAAAGTGGAAGCCTGATCCCATACAGAA (SEQ | RTT |
| | ID NO: 2518) | |
| 2519 | TCCAAAGTGGAAGCCTGATCCCATACAGAA (SEQ | RTT |
| | ID NO: 2519) | |
| 2520 | TTCCAAAGTGGAAGCCTGATCCCATACAGAA | RTT |
| | (SEQ ID NO: 2520) | |
| 2521 | TTTCCAAAGTGGAAGCCTGATCCCATACAGAA | RTT |
| | (SEQ ID NO: 2521) | |
| 2522 | GTTTCCAAAGTGGAAGCCTGATCCCATACAGAA | RTT |
| | (SEQ ID NO: 2522) | |
| 2523 | GGTTTCCAAAGTGGAAGCCTGATCCCATACAGA | RTT |
| | A (SEQ ID NO: 2523) | |
| 2524 | GGGTTTCCAAAGTGGAAGCCTGATCCCATACAG | RTT |
| | AA (SEQ ID NO: 2524) | |
| 2525 | TGGGTTTCCAAAGTGGAAGCCTGATCCCATACAG | RTT |
| | AA (SEQ ID NO: 2525) | |
| 2526 | CTGGGTTTCCAAAGTGGAAGCCTGATCCCATACA | RTT |
| | GAA (SEQ ID NO: 2526) | |
| 2527 | GCTGGGTTTCCAAAGTGGAAGCCTGATCCCATAC | RTT |
| | AGAA (SEQ ID NO: 2527) | |
| 2528 | TGCTGGGTTTCCAAAGTGGAAGCCTGATCCCATA | RTT |
| | CAGAA (SEQ ID NO: 2528) | |
| 2529 | TTGCTGGGTTTCCAAAGTGGAAGCCTGATCCCAT | RTT |
| | ACAGAA (SEQ ID NO: 2529) | |
| 1 | AACTGAGCCACGTGAAGACA (SEQ ID NO: 4) | PE3 ngRNA spacer (GG PAM) |
| 382 | AAGCCTGATCCCATACAGAA (SEQ ID NO: 382) | PE3b ngRNA spacer (AG PAM) |
| 195 | ACATCTTCCTTAGGAACTCC (SEQ ID NO: 195) | PE3 ngRNA spacer (TG PAM) |
| 196 | ACTACTTACCATCTCTTTCA (SEQ ID NO: 196) | PE3 ngRNA spacer (AG PAM) |
| 197 | ACTCCTGGAACTGAGCCACG (SEQ ID NO: 197) | PE3 ngRNA spacer (TG PAM) |
| 199 | AGGCTTCATACATCTTCCTT (SEQ ID NO: 199) | PE3 ngRNA spacer (AG PAM) |
| 200 | CATCTTCCTTAGGAACTCCT (SEQ ID NO: 200) | PE3 ngRNA spacer (GG PAM) |
| 201 | CCACGTGAAGACAGGTGTCT (SEQ ID NO: 201) | PE3 ngRNA spacer (TG PAM) |
| 202 | CCTGGAACTGAGCCACGTGA (SEQ ID NO: 202) | PE3 ngRNA spacer (AG PAM) |
| 203 | CGTGAAGACAGGTGTCTTGC (SEQ ID NO: 203) | PE3 ngRNA spacer (TG PAM) |
| 204 | CTACTTACCATCTCTTTCAA (SEQ ID NO: 204) | PE3 ngRNA spacer (GG PAM) |
| 205 | CTGAGCCACGTGAAGACAGG (SEQ ID NO: 205) | PE3 ngRNA spacer (TG PAM) |
| 206 | CTTAGGAACTCCTGGAACTG (SEQ ID NO: 206) | PE3 ngRNA spacer (AG PAM) |
| 208 | GAACTCCTGGAACTGAGCCA (SEQ ID NO: 208) | PE3 ngRNA spacer (CG PAM) |
| 209 | GAACTGAGCCACGTGAAGAC (SEQ ID NO: 209) | PE3 ngRNA spacer (AG PAM) |
| 210 | GGCTTCATACATCTTCCTTA (SEQ ID NO: 210) | PE3 ngRNA spacer (GG PAM) |
| 211 | GGGTTTCCAAAGTGGAAGCC (SEQ ID NO: 211) | PE3 ngRNA spacer (TG PAM) |
| 212 | GTCTTGCTGGGTTTCCAAAG (SEQ ID NO: 212) | PE3 ngRNA spacer (TG PAM) |
| 213 | GTGAAGACAGGTGTCTTGCT (SEQ ID NO: 213) | PE3 ngRNA spacer (GG PAM) |
| 384 | GTGGAAGCCTGATCCCATAC (SEQ ID NO: 384) | PE3b ngRNA spacer (AG PAM) |
| 214 | GTGTCTTGCTGGGTTTCCAA (SEQ ID NO: 214) | PE3 ngRNA spacer (AG PAM) |
| 215 | TCCTTAGGAACTCCTGGAAC (SEQ ID NO: 215) | PE3 ngRNA spacer (TG PAM) |
| 216 | TCTTGCTGGGTTTCCAAAGT (SEQ ID NO: 216) | PE3 ngRNA spacer (GG PAM) |
| 217 | TGAAGACAGGTGTCTTGCTG (SEQ ID NO: 217) | PE3 ngRNA spacer (GG PAM) |
| 218 | TGCTGGGTTTCCAAAGTGGA (SEQ ID NO: 218) | PE3 ngRNA spacer (AG PAM) |
| 323 | GTCTTGCTGGGTTTCCAAAGGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 323) | |
| 324 | GTGAAGACAGGTGTCTTGCTGTTTTAGAGCTAGA | PE3 ngRNA (GG PAM) |
| | AATAGCAAGTTAAAATAAGGCTAGTCCGTTATCA | |
| | ACTTGAAAAAGTGGCACCGAGTCGGTGC (SEQ ID | |
| | NO: 324) | |
| 325 | CGTGAAGACAGGTGTCTTGCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 325) | |
| 326 | GAACTGAGCCACGTGAAGACGTTTTAGAGCTAG | PE3 ngRNA (AG PAM) |
| | AAATAGCAAGTTAAAATAAGGCTAGTCCGTTATC | |
| | AACTTGAAAAAGTGGCACCGAGTCGGTGC (SEQ | |
| | ID NO: 326) | |
| 327 | ACATCTTCCTTAGGAACTCCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 327) | |
| 328 | AGGCTTCATACATCTTCCTTGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 328) | |
| 329 | ACTACTTACCATCTCTTTCAGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 329) | |
| 330 | GTCTTGCTGGGTTTCCAAAGGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 330) | |
| 331 | GTGAAGACAGGTGTCTTGCTGTTTTAGAGCTAGA | PE3 ngRNA (GG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 331) | |
| 332 | GAACTGAGCCACGTGAAGACGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 332) | |
| 333 | GCGTGAAGACAGGTGTCTTGCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 333) | |
| 334 | GACATCTTCCTTAGGAACTCCGTTTTAGAGCTAG | PE3 ngRNA (TG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 334) | |
| 335 | GAGGCTTCATACATCTTCCTTGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 335) | |
| 336 | GACTACTTACCATCTCTTTCAGTTTTAGAGCTAG | PE3 ngRNA (AG PAM), contains end |
| | AAATAGCAAGTTAAAATAAGGCTAGTCCGTTATC | adaptations |
| | AACTTGAAAAAGTGGCACCGAGTCGGTGCTTTT | |
| | (SEQ ID NO: 336) | |
| 337 | GTCTTGCTGGGTTTCCAAAGGTTTTAGAGCTAGA | PE3 ngRNA (TG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 337) | |
| 338 | GTGAAGACAGGTGTCTTGCTGTTTTAGAGCTAGA | PE3 ngRNA (GG PAM), contains linker |
| | AATAGCAAGTTAAAATAAGGCTAGTCCGTTATCA | sequence AACATTGA; hairpin SEQ ID |
| | ACTTGAAAAAGTGGCACCGAGTCGGTGCAACAT | NO: 3591; end adaptations |
| | TGACGCGTCTCTACGTGGGGGCGCGTTTT (SEQ | |
| | ID NO: 338 | |
| 339 | GAACTGAGCCACGTGAAGACGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 339) | |
| 340 | GCGTGAAGACAGGTGTCTTGCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 340) | |
| 341 | GACATCTTCCTTAGGAACTCCGTTTTAGAGCTAG | PE3 ngRNA (TG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 341) | |
| 342 | GAGGCTTCATACATCTTCCTTGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 342) | |
| 343 | GACTACTTACCATCTCTTTCAGTTTTAGAGCTAG | PE3 ngRNA (AG PAM), contains linker |
| | AAATAGCAAGTTAAAATAAGGCTAGTCCGTTATC | sequence AACATTGA; hairpin SEQ ID |
| | AACTTGAAAAAGTGGCACCGAGTCGGTGCAACA | NO: 3591; end adaptations |
| | TTGACGCGTCTCTACGTGGGGGCGCGTTTT (SEQ | |
| | ID NO: 343) | |

TABLE 35

| Sequence | | |
|---|---|---|
| Number | Sequence | Description |
| 2530 | GTCTTGGGTATGCACCT (SEQ ID NO: 2530) | PEgRNA spacer (ATAGAT PAM) |
| 2531 | GGTCTTGGGTATGCACCT (SEQ ID NO: 2531) | PEgRNA spacer (ATAGAT PAM) |
| 2532 | AGGTCTTGGGTATGCACCT (SEQ ID NO: 2532) | PERNA spacer (ATAGAT PAM) |
| 2533 | AAGGTCTTGGGTATGCACCT (SEQ ID NO: 2533) | PEgRNA spacer (ATAGAT PAM) |
| 2534 | CAAGGTCTTGGGTATGCACCT (SEQ ID NO: 2534) | PEgRNA spacer (ATAGAT PAM) |
| 2535 | TCAAGGTCTTGGGTATGCACCT (SEQ ID NO: 2535) | PEgRNA spacer (ATAGAT PAM) |
| 2536 | TGCAT | PBS |
| 2537 | TGCATA | PBS |
| 2538 | TGCATAC | PBS |
| 2539 | TGCATACC | PBS |
| 2540 | TGCATACCC | PBS |
| 2541 | TGCATACCCA (SEQ ID NO: 2541) | PBS |
| 2542 | TGCATACCCAA (SEQ ID NO: 2542) | PBS |
| 2543 | TGCATACCCAAG (SEQ ID NO: 2543) | PBS |
| 2544 | TGCATACCCAAGA (SEQ ID NO: 2544) | PBS |
| 2545 | TGCATACCCAAGAC (SEQ ID NO: 2545) | PBS |
| 2546 | TGCATACCCAAGACC (SEQ ID NO: 2546) | PBS |
| 2547 | TGCATACCCAAGACCT (SEQ ID NO: 2547) | PBS |
| 2548 | TGCATACCCAAGACCTT (SEQ ID NO: 2548) | PBS |
| 2549 | TGCATACCCAAGACCTTG (SEQ ID NO: 2549) | PBS |
| 2550 | TGCATACCCAAGACCTTGA (SEQ ID NO: 2550) | PBS |
| 2551 | TCACATTTTTAAGCAAACCAGGATAGTAATCTAT | RTT*1 |
| | AGG (SEQ ID NO: 2551) | |
| 2552 | TCACATTTTTAAGCAAACCAGGATAGTAGTCTAT | RTT*1,2 (ATAGAT-to-ATAGAC PAM |
| | AGG (SEQ ID NO: 2552) | silencing edit) |
| 2553 | CTCACATTTTTAAGCAAACCAGGATAGTAATCTA | RTT*1 |
| | TAGG (SEQ ID NO: 2553) | |
| 2554 | CTCACATTTTTAAGCAAACCAGGATAGTAGTCTA | RTT*1,2 (ATAGAT-to-ATAGAC PAM |
| | TAGG (SEQ ID NO: 2554) | silencing edit) |
| 2555 | ACTCACATTTTTAAGCAAACCAGGATAGTAATCT | RTT*1 |
| | ATAGG (SEQ ID NO: 2555) | |
| 2556 | ACTCACATTTTTAAGCAAACCAGGATAGTAGTCT | RTT*1,2 (ATAGAT-to-ATAGAC PAM |
| | ATAGG (SEQ ID NO: 2556) | silencing edit) |
| 2557 | TACTCACATTTTTAAGCAAACCAGGATAGTAATC | RTT*1 |
| | TATAGG (SEQ ID NO: 2557) | |
| 2558 | TACTCACATTTTTAAGCAAACCAGGATAGTAGTC | RTT*1,2 (ATAGAT-to-ATAGAC PAM |
| | TATAGG (SEQ ID NO: 2558) | silencing edit) |
| 2559 | AAACATCTCTTCTGGAGGAC (SEQ ID NO: 2559) | PE3 ngRNA spacer (TGAAAT PAM) |
| 2560 | ACTCACATTTTTAAGCAAAC (SEQ ID NO: 2560) | PE3b*1 ngRNA spacer (CAGGAT PAM) |
| 2561 | ATTTTTAAGCAAACCAGGAT (SEQ ID NO: 2561) | PE3 ngRNA spacer (AGTAAT PAM) |
| 2562 | CACATTTTTAAGCAAACCAG (SEQ ID NO: 2562) | PE3 ngRNA spacer (GATAGT PAM) |
| 2563 | CATTAAACATTTCAAAAGTG (SEQ ID NO: 2563) | PE3 ngRNA spacer (ATAAAT PAM) |
| 2564 | CATTCAGCATTAAACATTTC (SEQ ID NO: 2564) | PE3 ngRNA spacer (AAAAGT PAM) |
| 2565 | CATTTCAAAAGTGATAAATT (SEQ ID NO: 2565) | PE3 ngRNA spacer (TTAAAT PAM) |
| 2566 | GCAAACCAGGATAGTAATCT (SEQ ID NO: 2566) | PE3 ngRNA spacer (ATAGGT PAM) |
| 2567 | GCAAACCAGGATAGTAGTCT (SEQ ID NO: 2567) | PE3b*2 ngRNA spacer (ATAGGT PAM) |
| 2568 | TATTTCCATTTACTCTTTTT (SEQ ID NO: 2568) | PE3 ngRNA spacer (GCTGAT PAM) |
| 2569 | TCAGCATTAAACATTTCAAA (SEQ ID NO: 2569) | PE3 ngRNA spacer (AGTGAT PAM) |

TABLE 36

| Sequence | | |
|---|---|---|
| Number | Sequence | Description |
| 2570 | ATTAAACATTTCAAAAG (SEQ ID NO: 2570) | PEgRNA spacer (TG; TGA PAM) |
| 2571 | CATTAAACATTTCAAAAG (SEQ ID NO: 2571) | PEgRNA spacer (TG; TGA PAM) |
| 2572 | GCATTAAACATTTCAAAAG (SEQ ID NO: 2572) | PEgRNA spacer (TG; TGA PAM) |
| 2573 | AGCATTAAACATTTCAAAAG (SEQ ID NO: 2573) | PEgRNA spacer (TG; TGA PAM) |
| 2574 | CAGCATTAAACATTTCAAAAG (SEQ ID NO: 2574) | PEgRNA spacer (TG; TGA PAM) |
| 2575 | TCAGCATTAAACATTTCAAAAG (SEQ ID NO: | PEgRNA spacer (TG; TGA PAM) |
| | 2575) | |
| 2576 | TTGAA | PBS |
| 2577 | TTGAAA | PBS |
| 2578 | TTGAAAT | PBS |
| 2579 | TTGAAATG | PBS |
| 2580 | TTGAAATGT | PBS |
| 2581 | TTGAAATGTT (SEQ ID NO: 2581) | PBS |
| 2582 | TTGAAATGTTT (SEQ ID NO: 2582) | PBS |
| 2583 | TTGAAATGTTTA (SEQ ID NO: 2583) | PBS |
| 2584 | TTGAAATGTTTAA (SEQ ID NO: 2584) | PBS |
| 2585 | TTGAAATGTTTAAT (SEQ ID NO: 2585) | PBS |
| 2586 | TTGAAATGTTTAATG (SEQ ID NO: 2586) | PBS |
| 2587 | TTGAAATGTTTAATGC (SEQ ID NO: 2587) | PBS |
| 2588 | TTGAAATGTTTAATGCT (SEQ ID NO: 2588) | PBS |
| 2589 | TTGAAATGTTTAATGCTG (SEQ ID NO: 2589) | PBS |
| 2590 | TTGAAATGTTTAATGCTGA (SEQ ID NO: 2590) | PBS |
| 2591 | AGTATTTAAAATTTATCACTT (SEQ ID NO: 2591) | RTT |
| 2592 | GAGTATTTAAAATTTATCACTT (SEQ ID NO: 2592) | RTT |
| 2593 | TGAGTATTTAAAATTTATCACTT (SEQ ID NO: | RTT |
| | 2593) | |
| 2594 | GTGAGTATTTAAAATTTATCACTT (SEQ ID NO: | RTT |
| | 2594) | |
| 2595 | TGTGAGTATTTAAAATTTATCACTT (SEQ ID NO: | RTT |
| | 2595) | |
| 2596 | ATGTGAGTATTTAAAATTTATCACTT (SEQ ID NO: | RTT |
| | 2596) | |
| 2597 | AATGTGAGTATTTAAAATTTATCACTT (SEQ ID | RTT |
| | NO: 2597) | |
| 2598 | AAATGTGAGTATTTAAAATTTATCACTT (SEQ ID | RTT |
| | NO: 2598) | |
| 2599 | AAAATGTGAGTATTTAAAATTTATCACTT (SEQ ID | RTT |
| | NO: 2599) | |
| 2600 | AAAAATGTGAGTATTTAAAATTTATCACTT (SEQ | RTT |
| | ID NO: 2600) | |
| 2601 | TAAAAATGTGAGTATTTAAAATTTATCACTT | RTT |
| | (SEQ ID NO: 2601) | |
| 2602 | TTAAAAATGTGAGTATTTAAAATTTATCACTT | RTT |
| | (SEQ ID NO: 2602) | |
| 2603 | CTTAAAAATGTGAGTATTTAAAATTTATCACTT | RTT |
| | (SEQ ID NO: 2603) | |
| 2604 | GCTTAAAAATGTGAGTATTTAAAATTTATCACTT | RTT |
| | (SEQ ID NO: 2604) | |
| 2605 | TGCTTAAAAATGTGAGTATTTAAAATTTATCACT | RTT |
| | T (SEQ ID NO: 2605) | |
| 2606 | TTGCTTAAAAATGTGAGTATTTAAAATTTATCAC | RTT |
| | TT (SEQ ID NO: 2606) | |
| 2607 | TTTGCTTAAAAATGTGAGTATTTAAAATTTATCA | RTT |
| | CTT (SEQ ID NO: 2607) | |
| 2608 | GTTTGCTTAAAAATGTGAGTATTTAAAATTTATC | RTT |
| | ACTT (SEQ ID NO: 2608) | |
| 2609 | GGTTTGCTTAAAAATGTGAGTATTTAAAATTTAT | RTT |
| | CACTT (SEQ ID NO: 2609) | |
| 2610 | TGGTTTGCTTAAAAATGTGAGTATTTAAAATTTA | RTT |
| | TCACTT (SEQ ID NO: 2610) | |
| 2611 | AAGTTGCTCTTTTCACTCAA (SEQ ID NO: 2611) | PE3 ngRNA spacer (GG PAM) |
| 2612 | ATACTTTCAGCACTCAGATT (SEQ ID NO: 2612) | PE3 ngRNA spacer (TG; TGA PAM) |
| 2613 | CACCTATAGATTACTATCCT (SEQ ID NO: 2613) | PE3 ngRNA spacer (GG PAM) |
| 2614 | CTCAGATTTGATAAAGAAGT (SEQ ID NO: 2614) | PE3 ngRNA spacer (TG PAM) |
| 2615 | CTCATATACTTTCAGCACTC (SEQ ID NO: 2615) | PE3 ngRNA spacer (AG; AGA PAM) |
| 2616 | CTCTTTTCACTCAAGGTCTT (SEQ ID NO: 2616) | PE3 ngRNA spacer (GG PAM) |
| 2617 | CTGGTTTGCTTAAAAATGTG (SEQ ID NO: 2617) | PE3 ngRNA spacer (TG PAM) |
| 2618 | GAAGTTGCTCTTTTCACTCA (SEQ ID NO: 2618) | PE3 ngRNA spacer (AG PAM) |
| 2619 | GCACCTATAGATTACTATCC (SEQ ID NO: 2619) | PE3 ngRNA spacer (TG PAM) |
| 2620 | GCACTCAGATTTGATAAAGA (SEQ ID NO: 2620) | PE3 ngRNA spacer (AG PAM) |
| 2621 | GCTCTTTTCACTCAAGGTCT (SEQ ID NO: 2621) | PE3 ngRNA spacer (TG PAM) |
| 2622 | GGTCTTGGGTATGCACCTAT (SEQ ID NO: 2622) | PE3 ngRNA spacer (AG; AGA PAM) |
| 2623 | GTATTATCTCATATACTTTC (SEQ ID NO: 2623) | PE3 ngRNA spacer (AG PAM) |
| 2624 | TATAGATTACTATCCTGGTT (SEQ ID NO: 2624) | PE3 ngRNA spacer (TG PAM) |
| 2625 | TATCCTGGTTTGCTTAAAAA (SEQ ID NO: 2625) | PE3 ngRNA spacer (TG PAM) |
| 2626 | TATTTAAAATTTATCACTTT (SEQ ID NO: 2626) | PE3 ngRNA spacer (TG; TGA PAM) |
| 2627 | TCAGCACTCAGATTTGATAA (SEQ ID NO: 2627) | PE3 ngRNA spacer (AG; AGA PAM) |
| 2628 | TCCTGGTTTGCTTAAAAATG (SEQ ID NO: 2628) | PE3 ngRNA spacer (TG PAM) |
| 2629 | TCTTTTCACTCAAGGTCTTG (SEQ ID NO: 2629) | PE3 ngRNA spacer (GG PAM) |
| 2630 | TTCACTCAAGGTCTTGGGTA (SEQ ID NO: 2630) | PE3 ngRNA spacer (TG PAM) |

TABLE 37

| Sequence | | |
|---|---|---|
| Number | Sequence | Description |
| 2631 | TCTTGGGTATGCACCTA (SEQ ID NO: 2631) | PEgRNA spacer (TAG PAM) |
| 2632 | GTCTTGGGTATGCACCTA (SEQ ID NO: 2632) | PEgRNA spacer (TAG PAM) |
| 2633 | GGTCTTGGGTATGCACCTA (SEQ ID NO: 2633) | PERNA spacer (TAG PAM) |
| 2634 | AGGTCTTGGGTATGCACCTA (SEQ ID NO: 2634) | PERNA spacer (TAG PAM) |
| 2635 | AAGGTCTTGGGTATGCACCTA (SEQ ID NO: 2635) | PEgRNA spacer (TAG PAM) |
| 2636 | CAAGGTCTTGGGTATGCACCTA (SEQ ID NO: 2636) | PEgRNA spacer (TAG PAM) |
| 2637 | GTGCA | PBS |
| 2638 | GTGCAT | PBS |
| 2639 | GTGCATA | PBS |
| 2640 | GTGCATAC | PBS |
| 2641 | GTGCATACC | PBS |
| 2642 | GTGCATACCC (SEQ ID NO: 2642) | PBS |
| 2643 | GTGCATACCCA (SEQ ID NO: 2643) | PBS |
| 2644 | GTGCATACCCAA (SEQ ID NO: 2644) | PBS |
| 2645 | GTGCATACCCAAG (SEQ ID NO: 2645) | PBS |
| 2646 | GTGCATACCCAAGA (SEQ ID NO: 2646) | PBS |
| 2647 | GTGCATACCCAAGAC (SEQ ID NO: 2647) | PBS |
| 2648 | GTGCATACCCAAGACC (SEQ ID NO: 2648) | PBS |
| 2649 | GTGCATACCCAAGACCT (SEQ ID NO: 2649) | PBS |
| 2650 | GTGCATACCCAAGACCTT (SEQ ID NO: 2650) | PBS |
| 2651 | GTGCATACCCAAGACCTTG (SEQ ID NO: 2651) | PBS |
| 2652 | TCACATTTTTAAGCAAACCAGGATAGTAATCAAT | RTT*1,2,4 (TAG-to-TTG PAM silencing edit) |
| | AG (SEQ ID NO: 2652) | |
| 2653 | TCACATTTTTAAGCAAACCAGGATAGTAATCGAT | RTT*1,3,4 (TAG-to-TCG PAM silencing edit) |
| | AG (SEQ ID NO: 2653) | |
| 2654 | TCACATTTTTAAGCAAACCAGGATAGTAATCTAT | RTT*1,4 |
| | AG (SEQ ID NO: 2654) | |
| 2655 | CTCACATTTTTAAGCAAACCAGGATAGTAATCAA | RTT*1,2,4 (TAG-to-TTG PAM silencing edit) |
| | TAG (SEQ ID NO: 2655) | |
| 2656 | CTCACATTTTTAAGCAAACCAGGATAGTAATCGA | RTT*1,3,4 (TAG-to-TCG PAM silencing edit) |
| | TAG (SEQ ID NO: 2656) | |
| 2657 | CTCACATTTTTAAGCAAACCAGGATAGTAATCTA | RTT*1,4 |
| | TAG (SEQ ID NO: 2657) | |
| 2658 | ACTCACATTTTTAAGCAAACCAGGATAGTAATCA | RTT*1,2,4 (TAG-to-TTG PAM silencing edit) |
| | ATAG (SEQ ID NO: 2658) | |
| 2659 | ACTCACATTTTTAAGCAAACCAGGATAGTAATCG | RTT*1,3,4 (TAG-to-TCG PAM silencing edit) |
| | ATAG (SEQ ID NO: 2659) | |
| 2660 | ACTCACATTTTTAAGCAAACCAGGATAGTAATCT | RTT*1,4 |
| | ATAG (SEQ ID NO: 2660) | |
| 2661 | TACTCACATTTTTAAGCAAACCAGGATAGTAATC | RTT*1,2,4 (TAG-to-TTG PAM silencing edit) |
| | AATAG (SEQ ID NO: 2661) | |
| 2662 | TACTCACATTTTTAAGCAAACCAGGATAGTAATC | RTT*1,3,4 (TAG-to-TCG PAM silencing edit) |
| | GATAG (SEQ ID NO: 2662) | |
| 2663 | TACTCACATTTTTAAGCAAACCAGGATAGTAATC | RTT*1,4 |
| | TATAG (SEQ ID NO: 2663) | |
| 2664 | ATACTCACATTTTTAAGCAAACCAGGATAGTAAT | RTT*1,2,4 (TAG-to-TTG PAM silencing edit) |
| | CAATAG (SEQ ID NO: 2664) | |
| 2665 | ATACTCACATTTTTAAGCAAACCAGGATAGTAAT | RTT*1,3,4 (TAG-to-TCG PAM silencing edit) |
| | CGATAG (SEQ ID NO: 2665) | |
| 2666 | ATACTCACATTTTTAAGCAAACCAGGATAGTAAT | RTT*1,4 |
| | CTATAG (SEQ ID NO: 2666) | |
| 2560 | ACTCACATTTTTAAGCAAAC (SEQ ID NO: 2560) | PE3b*1 ngRNA spacer (CAG PAM) |
| 2667 | CAAACCAGGATAGTAATCAA (SEQ ID NO: 2667) | PE3b*2 ngRNA spacer (TAG PAM) |
| 2668 | CAAACCAGGATAGTAATCGA (SEQ ID NO: 2668) | PE3b*3 ngRNA spacer (TAG PAM) |
| 2669 | CAAACCAGGATAGTAATCTA (SEQ ID NO: 2669) | PE3 ngRNA spacer (TAG PAM) |
| 2670 | CATTTTTAAGCAAACCAGGA (SEQ ID NO: 2670) | PE3 ngRNA spacer (TAG PAM) |
| 2671 | CTTTTTGCTGATGGCACATT (SEQ ID NO: 2671) | PE3 ngRNA spacer (CAG PAM) |
| 2672 | TTCAGCATTAAACATTTCAA (SEQ ID NO: 2672) | PE3 ngRNA spacer (AAG PAM) |
| 2673 | TTTTAAATACTCACATTTTT (SEQ ID NO: 2673) | PE3b*4 ngRNA spacer (AAG PAM) |

TABLE 38

| Sequence | | |
|---|---|---|
| Number | Sequence | Description |
| 2674 | CACCTATAGATTACTAT (SEQ ID NO: 2674) | PEgRNA spacer (CCTGGT PAM) |
| 2675 | GCACCTATAGATTACTAT (SEQ ID NO: 2675) | PEgRNA spacer (CCTGGT PAM) |
| 2676 | TGCACCTATAGATTACTAT (SEQ ID NO: 2676) | PEgRNA spacer (CCTGGT PAM) |
| 2677 | ATGCACCTATAGATTACTAT (SEQ ID NO: 2677) | PEgRNA spacer (CCTGGT PAM) |
| 2678 | TATGCACCTATAGATTACTAT (SEQ ID NO: 2678) | PERNA spacer (CCTGGT PAM) |
| 2679 | GTATGCACCTATAGATTACTAT (SEQ ID NO: 2679) | PEgRNA spacer (CCTGGT PAM) |
| 2680 | GTAAT | PBS |
| 2681 | GTAATC | PBS |
| 2682 | GTAATCT | PBS |
| 2683 | GTAATCTA | PBS |
| 2684 | GTAATCTAT | PBS |
| 2685 | GTAATCTATA (SEQ ID NO: 2685) | PBS |
| 2686 | GTAATCTATAG (SEQ ID NO: 2686) | PBS |
| 2687 | GTAATCTATAGG (SEQ ID NO: 2687) | PBS |
| 2688 | GTAATCTATAGGT (SEQ ID NO: 2688) | PBS |
| 2689 | GTAATCTATAGGTG (SEQ ID NO: 2689) | PBS |
| 2690 | GTAATCTATAGGTGC (SEQ ID NO: 2690) | PBS |
| 2691 | GTAATCTATAGGTGCA (SEQ ID NO: 2691) | PBS |
| 2692 | GTAATCTATAGGTGCAT (SEQ ID NO: 2692) | PBS |
| 2693 | GTAATCTATAGGTGCATA (SEQ ID NO: 2693) | PBS |
| 2694 | GTAATCTATAGGTGCATAC (SEQ ID NO: 2694) | PBS |
| 2695 | TCACATTTTTAAGCAAACCAGGATA (SEQ ID NO: | RTT*1 |
| | 2695) | |
| 2696 | TCACATTTTTAAGCAACCCAGGATA (SEQ ID NO: | RTT*2,5,8 (CCTGGT-to-CCTGGG PAM |
| | 2696) | silencing edit) |
| 2697 | TCACATTTTTAAGCAAGCCAGGATA (SEQ ID NO: | RTT*3,6,9 (CCTGGT-to-CCTGGC PAM |
| | 2697) | silencing edit) |
| 2698 | TCACATTTTTAAGCAATCCAGGATA (SEQ ID NO: | RTT*4,7,10 (CCTGGT-to-CCTGGA PAM |
| | 2698) | silencing edit) |
| 2699 | CTCACATTTTTAAGCAAACCAGGATA (SEQ ID | RTT*1 |
| | NO: 2699) | |
| 2700 | CTCACATTTTTAAGCAACCCAGGATA (SEQ ID | RTT*2,5,8 (CCTGGT-to-CCTGGG PAM |
| | NO: 2700) | silencing edit) |
| 2701 | CTCACATTTTTAAGCAAGCCAGGATA (SEQ ID | RTT*3,6,9 (CCTGGT-to-CCTGGC PAM |
| | NO: 2701) | silencing edit) |
| 2702 | CTCACATTTTTAAGCAATCCAGGATA (SEQ ID | RTT*4,7,10 (CCTGGT-to-CCTGGA PAM |
| | NO: 2702) | silencing edit) |
| 2703 | ACTCACATTTTTAAGCAAACCAGGATA (SEQ ID | RTT*1 |
| | NO: 2703) | |
| 2704 | ACTCACATTTTTAAGCAACCCAGGATA (SEQ ID | RTT*2,5,8 (CCTGGT-to-CCTGGG PAM |
| | NO: 2704) | silencing edit) |
| 2705 | ACTCACATTTTTAAGCAAGCCAGGATA (SEQ ID | RTT*3,6,9 (CCTGGT-to-CCTGGC PAM |
| | NO: 2705) | silencing edit) |
| 2706 | ACTCACATTTTTAAGCAATCCAGGATA (SEQ ID | RTT*4,7,10 (CCTGGT-to-CCTGGA PAM |
| | NO: 2706) | silencing edit) |
| 2707 | TACTCACATTTTTAAGCAAACCAGGATA (SEQ ID | RTT*1 |
| | NO: 2707) | |
| 2708 | TACTCACATTTTTAAGCAACCCAGGATA (SEQ ID | RTT*2,5,8 (CCTGGT-to-CCTGGG PAM |
| | NO: 2708) | silencing edit) |
| 2709 | TACTCACATTTTTAAGCAAGCCAGGATA (SEQ ID | RTT*3,6,9 (CCTGGT-to-CCTGGC PAM |
| | NO: 2709) | silencing edit) |
| 2710 | TACTCACATTTTTAAGCAATCCAGGATA (SEQ ID | RTT*4,7,10 (CCTGGT-to-CCTGGA PAM |
| | NO: 2710) | silencing edit) |
| 2711 | ATACTCACATTTTTAAGCAAACCAGGATA (SEQ | RTT*1 |
| | ID NO: 2711) | |
| 2712 | ATACTCACATTTTTAAGCAACCCAGGATA (SEQ | RTT*2,5,8 (CCTGGT-to-CCTGGG PAM |
| | ID NO: 2712) | silencing edit) |
| 2713 | ATACTCACATTTTTAAGCAAGCCAGGATA (SEQ | RTT*3,6,9 (CCTGGT-to-CCTGGC PAM |
| | ID NO: 2713) | silencing edit) |
| 2714 | ATACTCACATTTTTAAGCAATCCAGGATA (SEQ | RTT*4,7,10 (CCTGGT-to-CCTGGA PAM |
| | ID NO: 2714) | silencing edit) |
| 2715 | AATACTCACATTTTTAAGCAAACCAGGATA (SEQ | RTT*1 |
| | ID NO: 2715) | |
| 2716 | AATACTCACATTTTTAAGCAACCCAGGATA (SEQ | RTT*2,5,8 (CCTGGT-to-CCTGGG PAM |
| | ID NO: 2716) | silencing edit) |
| 2717 | AATACTCACATTTTTAAGCAAGCCAGGATA (SEQ | RTT*3,6,9 (CCTGGT-to-CCTGGC PAM |
| | ID NO: 2717) | silencing edit) |
| 2718 | AATACTCACATTTTTAAGCAATCCAGGATA (SEQ | RTT*4,7,10 (CCTGGT-to-CCTGGA PAM |
| | ID NO: 2718) | silencing edit) |
| 2719 | AAATACTCACATTTTTAAGCAAACCAGGATA | RTT*1 |
| | (SEQ ID NO: 2719) | |
| 2720 | AAATACTCACATTTTTAAGCAACCCAGGATA | RTT*2,5,8 (CCTGGT-to-CCTGGG PAM |
| | (SEQ ID NO: 2720) | silencing edit) |
| 2721 | AAATACTCACATTTTTAAGCAAGCCAGGATA | RTT*3,6,9 (CCTGGT-to-CCTGGC PAM |
| | (SEQ ID NO: 2721) | silencing edit) |
| 2722 | AAATACTCACATTTTTAAGCAATCCAGGATA | RTT*4,7,10 (CCTGGT-to-CCTGGA PAM |
| | (SEQ ID NO: 2722) | silencing edit) |
| 2723 | TAAATACTCACATTTTTAAGCAAACCAGGATA | RTT*1 |
| | (SEQ ID NO: 2723) | |
| 2724 | TAAATACTCACATTTTTAAGCAACCCAGGATA | RTT*2,5,8 (CCTGGT-to-CCTGGG PAM |
| | (SEQ ID NO: 2724) | silencing edit) |
| 2725 | TAAATACTCACATTTTTAAGCAAGCCAGGATA | RTT*3,6,9 (CCTGGT-to-CCTGGC PAM |
| | (SEQ ID NO: 2725) | silencing edit) |
| 2726 | TAAATACTCACATTTTTAAGCAATCCAGGATA | RTT*4,7,10 (CCTGGT-to-CCTGGA PAM |
| | (SEQ ID NO: 2726) | silencing edit) |
| 2727 | TTAAATACTCACATTTTTAAGCAAACCAGGATA | RTT*1 |
| | (SEQ ID NO: 2727) | |
| 2728 | TTAAATACTCACATTTTTAAGCAACCCAGGATA | RTT*2,5,8 (CCTGGT-to-CCTGGG PAM |
| | (SEQ ID NO: 2728) | silencing edit) |
| 2729 | TTAAATACTCACATTTTTAAGCAAGCCAGGATA | RTT*3,6,9 (CCTGGT-to-CCTGGC PAM |
| | (SEQ ID NO: 2729) | silencing edit) |
| 2730 | TTAAATACTCACATTTTTAAGCAATCCAGGATA | RTT*4,7,10 (CCTGGT-to-CCTGGA PAM |
| | (SEQ ID NO: 2730) | silencing edit) |
| 2731 | TTTAAATACTCACATTTTTAAGCAAACCAGGATA | RTT*1 |
| | (SEQ ID NO: 2731) | |
| 2732 | TTTAAATACTCACATTTTTAAGCAACCCAGGATA | RTT*2,5,8 (CCTGGT-to-CCTGGG PAM |
| | (SEQ ID NO: 2732) | silencing edit) |
| 2733 | TTTAAATACTCACATTTTTAAGCAAGCCAGGATA | RTT*3,6,9 (CCTGGT-to-CCTGGC PAM |
| | (SEQ ID NO: 2733) | silencing edit) |
| 2734 | TTTAAATACTCACATTTTTAAGCAATCCAGGATA | RTT*4,7,10 (CCTGGT-to-CCTGGA PAM |
| | (SEQ ID NO: 2734) | silencing edit) |
| 2735 | TTTTAAATACTCACATTTTTAAGCAAACCAGGAT | RTT*1 |
| | A (SEQ ID NO: 2735) | |
| 2736 | TTTTAAATACTCACATTTTTAAGCAACCCAGGAT | RTT*2,5,8 (CCTGGT-to-CCTGGG PAM |
| | A (SEQ ID NO: 2736) | silencing edit) |
| 2737 | TTTTAAATACTCACATTTTTAAGCAAGCCAGGAT | RTT*3,6,9 (CCTGGT-to-CCTGGC PAM |
| | A (SEQ ID NO: 2737) | silencing edit) |
| 2738 | TTTTAAATACTCACATTTTTAAGCAATCCAGGAT | RTT*4,7,10 (CCTGGT-to-CCTGGA PAM |
| | A (SEQ ID NO: 2738) | silencing edit) |
| 2739 | ATTTTAAATACTCACATTTTTAAGCAAACCAGGA | RTT*1 |
| | TA (SEQ ID NO: 2739) | |
| 2740 | ATTTTAAATACTCACATTTTTAAGCAACCCAGGA | RTT*2,5,8 (CCTGGT-to-CCTGGG PAM |
| | TA (SEQ ID NO: 2740) | silencing edit) |
| 2741 | ATTTTAAATACTCACATTTTTAAGCAAGCCAGGA | RTT*3,6,9 (CCTGGT-to-CCTGGC PAM |
| | TA (SEQ ID NO: 2741) | silencing edit) |
| 2742 | ATTTTAAATACTCACATTTTTAAGCAATCCAGGA | RTT*4,7,10 (CCTGGT-to-CCTGGA PAM |
| | TA (SEQ ID NO: 2742) | silencing edit) |
| 2743 | AATTTTAAATACTCACATTTTTAAGCAAACCAGG | RTT*1 |
| | ATA (SEQ ID NO: 2743) | |
| 2744 | AATTTTAAATACTCACATTTTTAAGCAACCCAGG | RTT*2,5,8 (CCTGGT-to-CCTGGG PAM |
| | ATA (SEQ ID NO: 2744) | silencing edit) |
| 2745 | AATTTTAAATACTCACATTTTTAAGCAAGCCAGG | RTT*3,6,9 (CCTGGT-to-CCTGGC PAM |
| | ATA (SEQ ID NO: 2745) | silencing edit) |
| 2746 | AATTTTAAATACTCACATTTTTAAGCAATCCAGG | RTT*4,7,10 (CCTGGT-to-CCTGGA PAM |
| | ATA (SEQ ID NO: 2746) | silencing edit) |
| 2747 | AAATTTTAAATACTCACATTTTTAAGCAAACCAG | RTT*1 |
| | GATA (SEQ ID NO: 2747) | |
| 2748 | AAATTTTAAATACTCACATTTTTAAGCAACCCAG | RTT*2,5,8 (CCTGGT-to-CCTGGG PAM |
| | GATA (SEQ ID NO: 2748) | silencing edit) |
| 2749 | AAATTTTAAATACTCACATTTTTAAGCAAGCCAG | RTT*3,6,9 (CCTGGT-to-CCTGGC PAM |
| | GATA (SEQ ID NO: 2749) | silencing edit) |
| 2750 | AAATTTTAAATACTCACATTTTTAAGCAATCCAG | RTT*4,7,10 (CCTGGT-to-CCTGGA PAM |
| | GATA (SEQ ID NO: 2750) | silencing edit) |
| 2751 | TAAATTTTAAATACTCACATTTTTAAGCAAACCA | RTT*1 |
| | GGATA (SEQ ID NO: 2751) | |
| 2752 | TAAATTTTAAATACTCACATTTTTAAGCAACCCA | RTT*2,5,8 (CCTGGT-to-CCTGGG PAM |
| | GGATA (SEQ ID NO: 2752) | silencing edit) |
| 2753 | TAAATTTTAAATACTCACATTTTTAAGCAAGCCA | RTT*3,6,9 (CCTGGT-to-CCTGGC PAM |
| | GGATA (SEQ ID NO: 2753) | silencing edit) |
| 2754 | TAAATTTTAAATACTCACATTTTTAAGCAATCCA | RTT*4,7,10 (CCTGGT-to-CCTGGA PAM |
| | GGATA (SEQ ID NO: 2754) | silencing edit) |
| 2755 | ATAAATTTTAAATACTCACATTTTTAAGCAAACC | RTT*1 |
| | AGGATA (SEQ ID NO: 2755) | |
| 2756 | ATAAATTTTAAATACTCACATTTTTAAGCAACCC | RTT*2,5,8 (CCTGGT-to-CCTGGG PAM |
| | AGGATA (SEQ ID NO: 2756) | silencing edit) |
| 2757 | ATAAATTTTAAATACTCACATTTTTAAGCAAGCC | RTT*3,6,9 (CCTGGT-to-CCTGGC PAM |
| | AGGATA (SEQ ID NO: 2757) | silencing edit) |
| 2758 | ATAAATTTTAAATACTCACATTTTTAAGCAATCC | RTT*4,7,10 (CCTGGT-to-CCTGGA PAM |
| | AGGATA (SEQ ID NO: 2758) | silencing edit) |
| 2559 | AAACATCTCTTCTGGAGGAC (SEQ ID NO: 2559) | PE3 ngRNA spacer (TGAAAT PAM) |
| 2560 | ACTCACATTTTTAAGCAAAC (SEQ ID NO: 2560) | PE3b*1 ngRNA spacer (CAGGAT PAM) |
| 2759 | ACTCACATTTTTAAGCAACC (SEQ ID NO: 2759) | PE3b*2 ngRNA spacer (CAGGAT PAM) |
| 2760 | ACTCACATTTTTAAGCAAGC (SEQ ID NO: 2760) | PE3b*3 ngRNA spacer (CAGGAT PAM) |
| 2761 | ACTCACATTTTTAAGCAATC (SEQ ID NO: 2761) | PE3b*4 ngRNA spacer (CAGGAT PAM) |
| 2561 | ATTTTTAAGCAAACCAGGAT (SEQ ID NO: 2561) | PE3 ngRNA spacer (AGTAAT PAM) |
| 2762 | ATTTTTAAGCAACCCAGGAT (SEQ ID NO: 2762) | PE3b*5 ngRNA spacer (AGTAAT PAM) |
| 2763 | ATTTTTAAGCAAGCCAGGAT (SEQ ID NO: 2763) | PE3b*6 ngRNA spacer (AGTAAT PAM) |
| 2764 | ATTTTTAAGCAATCCAGGAT (SEQ ID NO: 2764) | PE3b*7 ngRNA spacer (AGTAAT PAM) |
| 2562 | CACATTTTTAAGCAAACCAG (SEQ ID NO: 2562) | PE3 ngRNA spacer (GATAGT PAM) |
| 2765 | CACATTTTTAAGCAACCCAG (SEQ ID NO: 2765) | PE3b*8 ngRNA spacer (GATAGT PAM) |
| 2766 | CACATTTTTAAGCAAGCCAG (SEQ ID NO: 2766) | PE3b*9 ngRNA spacer (GATAGT PAM) |
| 2767 | CACATTTTTAAGCAATCCAG (SEQ ID NO: 2767) | PE3b*10 ngRNA spacer (GATAGT PAM) |
| 2563 | CATTAAACATTTCAAAAGTG (SEQ ID NO: 2563) | PE3 ngRNA spacer (ATAAAT PAM) |
| 2564 | CATTCAGCATTAAACATTTC (SEQ ID NO: 2564) | PE3 ngRNA spacer (AAAAGT PAM) |
| 2565 | CATTTCAAAAGTGATAAATT (SEQ ID NO: 2565) | PE3 ngRNA spacer (TTAAAT PAM) |
| 2568 | TATTTCCATTTACTCTTTTT (SEQ ID NO: 2568) | PE3 ngRNA spacer (GCTGAT PAM) |
| 2569 | TCAGCATTAAACATTTCAAA (SEQ ID NO: 2569) | PE3 ngRNA spacer (AGTGAT PAM) |

TABLE 39

| Sequence | | |
|---|---|---|
| Number | Sequence | Description |
| 2768 | ACTATCCTGGTTTGCTT (SEQ ID NO: 2768) | PEgRNA spacer (AAAAAT PAM) |
| 2769 | TACTATCCTGGTTTGCTT (SEQ ID NO: 2769) | PEgRNA spacer (AAAAAT PAM) |
| 2770 | TTACTATCCTGGTTTGCTT (SEQ ID NO: 2770) | PEgRNA spacer (AAAAAT PAM) |
| 2771 | ATTACTATCCTGGTTTGCTT (SEQ ID NO: 2771) | PEgRNA spacer (AAAAAT PAM) |
| 2772 | GATTACTATCCTGGTTTGCTT (SEQ ID NO: 2772) | PEgRNA spacer (AAAAAT PAM) |
| 2773 | AGATTACTATCCTGGTTTGCTT (SEQ ID NO: 2773) | PEgRNA spacer (AAAAAT PAM) |
| 2774 | CAAAC | PBS |
| 2775 | CAAACC | PBS |
| 2776 | CAAACCA | PBS |
| 2777 | CAAACCAG | PBS |
| 2778 | CAAACCAGG | PBS |
| 2779 | CAAACCAGGA (SEQ ID NO: 2779) | PBS |
| 2780 | CAAACCAGGAT (SEQ ID NO: 2780) | PBS |
| 2781 | CAAACCAGGATA (SEQ ID NO: 2781) | PBS |
| 2782 | CAAACCAGGATAG (SEQ ID NO: 2782) | PBS |
| 2783 | CAAACCAGGATAGT (SEQ ID NO: 2783) | PBS |
| 2784 | CAAACCAGGATAGTA (SEQ ID NO: 2784) | PBS |
| 2785 | CAAACCAGGATAGTAA (SEQ ID NO: 2785) | PBS |
| 2786 | CAAACCAGGATAGTAAT (SEQ ID NO: 2786) | PBS |
| 2787 | CAAACCAGGATAGTAATC (SEQ ID NO: 2787) | PBS |
| 2788 | CAAACCAGGATAGTAATCT (SEQ ID NO: 2788) | PBS |
| 2789 | TCACATTTTTAAG (SEQ ID NO: 2789) | RTT |
| 2790 | TCACGTTTTTAAG (SEQ ID NO: 2790) | RTT (AAAAAT-to-AAAAAC PAM silencing |
| | | edit) |
| 2791 | CTCACATTTTTAAG (SEQ ID NO: 2791) | RTT |
| 2792 | CTCACGTTTTTAAG (SEQ ID NO: 2792) | RTT (AAAAAT-to-AAAAAC PAM silencing |
| | | edit) |
| 2793 | ACTCACATTTTTAAG (SEQ ID NO: 2793) | RTT |
| 2794 | ACTCACGTTTTTAAG (SEQ ID NO: 2794) | RTT (AAAAAT-to-AAAAAC PAM silencing |
| | | edit) |
| 2795 | TACTCACATTTTTAAG (SEQ ID NO: 2795) | RTT |
| 2796 | TACTCACGTTTTTAAG (SEQ ID NO: 2796) | RTT (AAAAAT-to-AAAAAC PAM silencing |
| | | edit) |
| 2797 | ATACTCACATTTTTAAG (SEQ ID NO: 2797) | RTT |
| 2798 | ATACTCACGTTTTTAAG (SEQ ID NO: 2798) | RTT (AAAAAT-to-AAAAAC PAM silencing |
| | | edit) |
| 2799 | AATACTCACATTTTTAAG (SEQ ID NO: 2799) | RTT |
| 2800 | AATACTCACGTTTTTAAG (SEQ ID NO: 2800) | RTT (AAAAAT-to-AAAAAC PAM silencing |
| | | edit) |
| 2801 | AAATACTCACATTTTTAAG (SEQ ID NO: 2801) | RTT |
| 2802 | AAATACTCACGTTTTTAAG (SEQ ID NO: 2802) | RTT (AAAAAT-to-AAAAAC PAM silencing |
| | | edit) |
| 2803 | TAAATACTCACATTTTTAAG (SEQ ID NO: 2803) | RTT |
| 2804 | TAAATACTCACGTTTTTAAG (SEQ ID NO: 2804) | RTT (AAAAAT-to-AAAAAC PAM silencing |
| | | edit) |
| 2805 | TTAAATACTCACATTTTTAAG (SEQ ID NO: 2805) | RTT |
| 2806 | TTAAATACTCACGTTTTTAAG (SEQ ID NO: 2806) | RTT (AAAAAT-to-AAAAAC PAM silencing |
| | | edit) |
| 2807 | TTTAAATACTCACATTTTTAAG (SEQ ID NO: 2807) | RTT |
| 2808 | TTTAAATACTCACGTTTTTAAG (SEQ ID NO: 2808) | RTT (AAAAAT-to-AAAAAC PAM silencing |
| | | edit) |
| 2809 | TTTTAAATACTCACATTTTTAAG (SEQ ID NO: | RTT |
| | 2809) | |
| 2810 | TTTTAAATACTCACGTTTTTAAG (SEQ ID NO: | RTT (AAAAAT-to-AAAAAC PAM silencing |
| | 2810) | edit) |
| 2811 | ATTTTAAATACTCACATTTTTAAG (SEQ ID NO: | RTT |
| | 2811) | |
| 2812 | ATTTTAAATACTCACGTTTTTAAG (SEQ ID NO: | RTT (AAAAAT-to-AAAAAC PAM silencing |
| | 2812) | edit) |
| 2813 | AATTTTAAATACTCACATTTTTAAG (SEQ ID NO: | RTT |
| | 2813) | |
| 2814 | AATTTTAAATACTCACGTTTTTAAG (SEQ ID NO: | RTT (AAAAAT-to-AAAAAC PAM silencing |
| | 2814) | edit) |
| 2815 | AAATTTTAAATACTCACATTTTTAAG (SEQ ID NO: | RTT |
| | 2815) | |
| 2816 | AAATTTTAAATACTCACGTTTTTAAG (SEQ ID NO: | RTT (AAAAAT-to-AAAAAC PAM silencing |
| | 2816) | edit) |
| 2817 | TAAATTTTAAATACTCACATTTTTAAG (SEQ ID | RTT |
| | NO: 2817) | |
| 2818 | TAAATTTTAAATACTCACGTTTTTAAG (SEQ ID | RTT (AAAAAT-to-AAAAAC PAM silencing |
| | NO: 2818) | edit) |
| 2819 | ATAAATTTTAAATACTCACATTTTTAAG (SEQ ID | RTT |
| | NO: 2819) | |
| 2820 | ATAAATTTTAAATACTCACGTTTTTAAG (SEQ ID | RTT (AAAAAT-to-AAAAAC PAM silencing |
| | NO: 2820) | edit) |
| 2821 | GATAAATTTTAAATACTCACATTTTTAAG (SEQ ID | RTT |
| | NO: 2821) | |
| 2822 | GATAAATTTTAAATACTCACGTTTTTAAG (SEQ ID | RTT (AAAAAT-to-AAAAAC PAM silencing |
| | NO: 2822) | edit) |
| 2823 | TGATAAATTTTAAATACTCACATTTTTAAG (SEQ | RTT |
| | ID NO: 2823) | |
| 2824 | TGATAAATTTTAAATACTCACGTTTTTAAG (SEQ | RTT (AAAAAT-to-AAAAAC PAM silencing |
| | ID NO: 2824) | edit) |
| 2825 | GTGATAAATTTTAAATACTCACATTTTTAAG (SEQ | RTT |
| | ID NO: 2825) | |
| 2826 | GTGATAAATTTTAAATACTCACGTTTTTAAG (SEQ | RTT (AAAAAT-to-AAAAAC PAM silencing |
| | ID NO: 2826) | edit) |
| 2827 | AGTGATAAATTTTAAATACTCACATTTTTAAG | RTT |
| | (SEQ ID NO: 2827) | |
| 2828 | AGTGATAAATTTTAAATACTCACGTTTTTAAG | RTT (AAAAAT-to-AAAAAC PAM silencing |
| | (SEQ ID NO: 2828) | edit) |
| 2829 | AAGTGATAAATTTTAAATACTCACATTTTTAAG | RTT |
| | (SEQ ID NO: 2829) | |
| 2830 | AAGTGATAAATTTTAAATACTCACGTTTTTAAG | RTT (AAAAAT-to-AAAAAC PAM silencing |
| | (SEQ ID NO: 2830) | edit) |
| 2831 | AAAGTGATAAATTTTAAATACTCACATTTTTAAG | RTT |
| | (SEQ ID NO: 2831) | |
| 2832 | AAAGTGATAAATTTTAAATACTCACGTTTTTAAG | RTT (AAAAAT-to-AAAAAC PAM silencing |
| | (SEQ ID NO: 2832) | edit) |
| 2833 | AAAAGTGATAAATTTTAAATACTCACATTTTTAA | RTT |
| | G (SEQ ID NO: 2833) | |
| 2834 | AAAAGTGATAAATTTTAAATACTCACGTTTTTAA | RTT (AAAAAT-to-AAAAAC PAM silencing |
| | G (SEQ ID NO: 2834) | edit) |
| 2835 | CAAAAGTGATAAATTTTAAATACTCACATTTTTA | RTT |
| | AG (SEQ ID NO: 2835) | |
| 2836 | CAAAAGTGATAAATTTTAAATACTCACGTTTTTA | RTT (AAAAAT-to-AAAAAC PAM silencing |
| | AG (SEQ ID NO: 2836) | edit) |
| 2837 | TCAAAAGTGATAAATTTTAAATACTCACATTTTT | RTT |
| | AAG (SEQ ID NO: 2837) | |
| 2838 | TCAAAAGTGATAAATTTTAAATACTCACGTTTTT | RTT (AAAAAT-to-AAAAAC PAM silencing |
| | AAG (SEQ ID NO: 2838) | edit) |
| 2839 | TTCAAAAGTGATAAATTTTAAATACTCACATTTT | RTT |
| | TAAG (SEQ ID NO: 2839) | |
| 2840 | TTCAAAAGTGATAAATTTTAAATACTCACGTTTT | RTT (AAAAAT-to-AAAAAC PAM silencing |
| | TAAG (SEQ ID NO: 2840) | edit) |
| 2841 | TTTCAAAAGTGATAAATTTTAAATACTCACATTT | RTT |
| | TTAAG (SEQ ID NO: 2841) | |
| 2842 | TTTCAAAAGTGATAAATTTTAAATACTCACGTTT | RTT (AAAAAT-to-AAAAAC PAM silencing |
| | TTAAG (SEQ ID NO: 2842) | edit) |
| 2843 | ATTTCAAAAGTGATAAATTTTAAATACTCACATT | RTT |
| | TTTAAG (SEQ ID NO: 2843) | |
| 2844 | ATTTCAAAAGTGATAAATTTTAAATACTCACGTT | RTT (AAAAAT-to-AAAAAC PAM silencing |
| | TTTAAG (SEQ ID NO: 2844) | edit) |
| 2559 | AAACATCTCTTCTGGAGGAC (SEQ ID NO: 2559) | PE3 ngRNA spacer (TGAAAT PAM) |
| 2563 | CATTAAACATTTCAAAAGTG (SEQ ID NO: 2563) | PE3 ngRNA spacer (ATAAAT PAM) |
| 2564 | CATTCAGCATTAAACATTTC (SEQ ID NO: 2564) | PE3 ngRNA spacer (AAAAGT PAM) |
| 2565 | CATTTCAAAAGTGATAAATT (SEQ ID NO: 2565) | PE3 ngRNA spacer (TTAAAT PAM) |
| 2845 | GTAAGAAGAGATAAACAAAG (SEQ ID NO: 2845) | PE3 ngRNA spacer (AAAAGT PAM) |
| 2568 | TATTTCCATTTACTCTTTTT (SEQ ID NO: 2568) | PE3 ngRNA spacer (GCTGAT PAM) |
| 2569 | TCAGCATTAAACATTTCAAA (SEQ ID NO: 2569) | PE3 ngRNA spacer (AGTGAT PAM) |

TABLE 40

| Sequence | | |
|---|---|---|
| Number | Sequence | Description |
| 2846 | CTATAGATTACTATCCT (SEQ ID NO: 2846) | PEgRNA spacer (GG PAM) |
| 2847 | CCTATAGATTACTATCCT (SEQ ID NO: 2847) | PERNA spacer (GG PAM) |
| 2848 | ACCTATAGATTACTATCCT (SEQ ID NO: 2848) | PEgRNA spacer (GG PAM) |
| 2613 | CACCTATAGATTACTATCCT (SEQ ID NO: 2613) | PEgRNA spacer (GG PAM) |
| 2849 | GCACCTATAGATTACTATCCT (SEQ ID NO: 2849) | PEgRNA spacer (GG PAM) |
| 2850 | TGCACCTATAGATTACTATCCT (SEQ ID NO: 2850) | PERNA spacer (GG PAM) |
| 2851 | ATAGT | PBS |
| 2852 | ATAGTA | PBS |
| 2853 | ATAGTAA | PBS |
| 2854 | ATAGTAAT | PBS |
| 2855 | ATAGTAATC | PBS |
| 2856 | ATAGTAATCT (SEQ ID NO: 2856) | PBS |
| 2857 | ATAGTAATCTA (SEQ ID NO: 2857) | PBS |
| 2858 | ATAGTAATCTAT (SEQ ID NO: 2858) | PBS |
| 2859 | ATAGTAATCTATA (SEQ ID NO: 2859) | PBS |
| 2860 | ATAGTAATCTATAG (SEQ ID NO: 2860) | PBS |
| 2861 | ATAGTAATCTATAGG (SEQ ID NO: 2861) | PBS |
| 2862 | ATAGTAATCTATAGGT (SEQ ID NO: 2862) | PBS |
| 2863 | ATAGTAATCTATAGGTG (SEQ ID NO: 2863) | PBS |
| 2864 | ATAGTAATCTATAGGTGC (SEQ ID NO: 2864) | PBS |
| 2865 | ATAGTAATCTATAGGTGCA (SEQ ID NO: 2865) | PBS |
| 2866 | TCACATTTTTAAGCAAACCAGG (SEQ ID NO: 2866) | RTT |
| 2867 | CTCACATTTTTAAGCAAACCAGG (SEQ ID NO: | RTT |
| | 2867) | |
| 2868 | ACTCACATTTTTAAGCAAACCAGG (SEQ ID NO: | RTT |
| | 2868) | |
| 2869 | TACTCACATTTTTAAGCAAACCAGG (SEQ ID NO: | RTT |
| | 2869) | |
| 2870 | ATACTCACATTTTTAAGCAAACCAGG (SEQ ID | RTT |
| | NO: 2870) | |
| 2871 | AATACTCACATTTTTAAGCAAACCAGG (SEQ ID | RTT |
| | NO: 2871) | |
| 2872 | AAATACTCACATTTTTAAGCAAACCAGG (SEQ ID | RTT |
| | NO: 2872) | |
| 2873 | TAAATACTCACATTTTTAAGCAAACCAGG (SEQ | RTT |
| | ID NO: 2873) | |
| 2874 | TTAAATACTCACATTTTTAAGCAAACCAGG (SEQ | RTT |
| | ID NO: 2874) | |
| 2875 | TTTAAATACTCACATTTTTAAGCAAACCAGG | RTT |
| | (SEQ ID NO: 2875) | |
| 2876 | TTTTAAATACTCACATTTTTAAGCAAACCAGG | RTT |
| | (SEQ ID NO: 2876) | |
| 2877 | ATTTTAAATACTCACATTTTTAAGCAAACCAGG | RTT |
| | (SEQ ID NO: 2877) | |
| 2878 | AATTTTAAATACTCACATTTTTAAGCAAACCAGG | RTT |
| | (SEQ ID NO: 2878) | |
| 2879 | AAATTTTAAATACTCACATTTTTAAGCAAACCAG | RTT |
| | G (SEQ ID NO: 2879) | |
| 2880 | TAAATTTTAAATACTCACATTTTTAAGCAAACCA | RTT |
| | GG (SEQ ID NO: 2880) | |
| 2881 | ATAAATTTTAAATACTCACATTTTTAAGCAAACC | RTT |
| | AGG (SEQ ID NO: 2881) | |
| 2882 | GATAAATTTTAAATACTCACATTTTTAAGCAAAC | RTT |
| | CAGG (SEQ ID NO: 2882) | |
| 2883 | TGATAAATTTTAAATACTCACATTTTTAAGCAAA | RTT |
| | CCAGG (SEQ ID NO: 2883) | |
| 2884 | GTGATAAATTTTAAATACTCACATTTTTAAGCAA | RTT |
| | ACCAGG (SEQ ID NO: 2884) | |
| 2885 | AAAAGTTAAACATCTCTTCT (SEQ ID NO: 2885) | PE3 ngRNA spacer (GG PAM) |
| 2559 | AAACATCTCTTCTGGAGGAC (SEQ ID NO: 2559) | PE3 ngRNA spacer (TG PAM) |
| 2886 | AAGTTAAACATCTCTTCTGG (SEQ ID NO: 2886) | PE3 ngRNA spacer (AG PAM) |
| 2573 | AGCATTAAACATTTCAAAAG (SEQ ID NO: 2573) | PE3 ngRNA spacer (TG PAM) |
| 2887 | AGTTAAACATCTCTTCTGGA (SEQ ID NO: 2887) | PE3 ngRNA spacer (GG PAM) |
| 2888 | ATATTTCCATTTACTCTTTT (SEQ ID NO: 2888) | PE3 ngRNA spacer (TG PAM) |
| 2561 | ATTTTTAAGCAAACCAGGAT (SEQ ID NO: 2561) | PE3 ngRNA spacer (AG PAM) |
| 2889 | CATTTACTCTTTTTGCTGAT (SEQ ID NO: 2889) | PE3 ngRNA spacer (GG PAM) |
| 2890 | CCATTTACTCTTTTTGCTGA (SEQ ID NO: 2890) | PE3 ngRNA spacer (TG PAM) |
| 2891 | CTCACATTTTTAAGCAAACC (SEQ ID NO: 2891) | PE3b ngRNA spacer (AG PAM) |
| 2892 | GAAAAGTTAAACATCTCTTC (SEQ ID NO: 2892) | PE3 ngRNA spacer (TG PAM) |
| 2893 | TCACATTTTTAAGCAAACCA (SEQ ID NO: 2893) | PE3b ngRNA spacer (GG PAM) |
| 2569 | TCAGCATTAAACATTTCAAA (SEQ ID NO: 2569) | PE3 ngRNA spacer (AG PAM) |
| 2894 | TTTAAATACTCACATTTTTA (SEQ ID NO: 2894) | PE3b ngRNA spacer (AG PAM) |
| 2895 | TTTCCATTTACTCTTTTTGC (SEQ ID NO: 2895) | PE3 ngRNA spacer (TG PAM) |
| 2896 | TTTTTGCTGATGGCACATTC (SEQ ID NO: 2896) | PE3 ngRNA spacer (AG PAM) |
| 2897 | AAGTTAAACATCTCTTCTGGGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2898 | GAAAAGTTAAACATCTCTTCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 2898) | |
| 2899 | AAGTTAAACATCTCTTCTGGGTTTTAGAGCTAGA | PE3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |
| 2900 | GAAAAGTTAAACATCTCTTCGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2900) | |

TABLE 41

| Sequence | | |
|---|---|---|
| Number | Sequence | Description |
| 2901 | TAAACATTTCAAAAGTG (SEQ ID NO: 2901) | PEgRNA spacer (ATAAAT PAM) |
| 2902 | TTAAACATTTCAAAAGTG (SEQ ID NO: 2902) | PEgRNA spacer (ATAAAT PAM) |
| 2903 | ATTAAACATTTCAAAAGTG (SEQ ID NO: 2903) | PEgRNA spacer (ATAAAT PAM) |
| 2563 | CATTAAACATTTCAAAAGTG (SEQ ID NO: 2563) | PERNA spacer (ATAAAT PAM) |
| 2904 | GCATTAAACATTTCAAAAGTG (SEQ ID NO: 2904) | PEgRNA spacer (ATAAAT PAM) |
| 2905 | AGCATTAAACATTTCAAAAGTG (SEQ ID NO: | PEgRNA spacer (ATAAAT PAM) |
| | 2905) | |
| 2906 | TTTTG | PBS |
| 2907 | TTTTGA | PBS |
| 2908 | TTTTGAA | PBS |
| 2909 | TTTTGAAA | PBS |
| 2910 | TTTTGAAAT | PBS |
| 2911 | TTTTGAAATG (SEQ ID NO: 2911) | PBS |
| 2912 | TTTTGAAATGT (SEQ ID NO: 2912) | PBS |
| 2913 | TTTTGAAATGTT (SEQ ID NO: 2913) | PBS |
| 2914 | TTTTGAAATGTTT (SEQ ID NO: 2914) | PBS |
| 2915 | TTTTGAAATGTTTA (SEQ ID NO: 2915) | PBS |
| 2916 | TTTTGAAATGTTTAA (SEQ ID NO: 2916) | PBS |
| 2917 | TTTTGAAATGTTTAAT (SEQ ID NO: 2917) | PBS |
| 2918 | TTTTGAAATGTTTAATG (SEQ ID NO: 2918) | PBS |
| 2919 | TTTTGAAATGTTTAATGC (SEQ ID NO: 2919) | PBS |
| 2920 | TTTTGAAATGTTTAATGCT (SEQ ID NO: 2920) | PBS |
| 2921 | AGTATTTAAAATTTATCAC (SEQ ID NO: 2921) | RTT |
| 2922 | GAGTATTTAAAATTTATCAC (SEQ ID NO: 2922) | RTT |
| 2923 | TGAGTATTTAAAATTTATCAC (SEQ ID NO: 2923) | RTT |
| 2924 | GTGAGTATTTAAAATTTATCAC (SEQ ID NO: 2924) | RTT |
| 2925 | TGTGAGTATTTAAAATTTATCAC (SEQ ID NO: | RTT |
| | 2925) | |
| 2926 | ATGTGAGTATTTAAAATTTATCAC (SEQ ID NO: | RTT |
| | 2926) | |
| 2927 | AATGTGAGTATTTAAAATTTATCAC (SEQ ID NO: | RTT |
| | 2927) | |
| 2928 | AAATGTGAGTATTTAAAATTTATCAC (SEQ ID | RTT |
| | NO: 2928) | |
| 2929 | AAAATGTGAGTATTTAAAATTTATCAC (SEQ ID | RTT |
| | NO: 2929) | |
| 2930 | AAAAATGTGAGTATTTAAAATTTATCAC (SEQ ID | RTT |
| | NO: 2930) | |
| 2931 | TAAAAATGTGAGTATTTAAAATTTATCAC (SEQ | RTT |
| | ID NO: 2931) | |
| 2932 | TTAAAAATGTGAGTATTTAAAATTTATCAC (SEQ | RTT |
| | ID NO: 2932) | |
| 2933 | CTTAAAAATGTGAGTATTTAAAATTTATCAC | RTT |
| | (SEQ ID NO: 2933) | |
| 2934 | GCTTAAAAATGTGAGTATTTAAAATTTATCAC | RTT |
| | (SEQ ID NO: 2934) | |
| 2935 | TGCTTAAAAATGTGAGTATTTAAAATTTATCAC | RTT |
| | (SEQ ID NO: 2935) | |
| 2936 | TTGCTTAAAAATGTGAGTATTTAAAATTTATCAC | RTT |
| | (SEQ ID NO: 2936) | |
| 2937 | TTTGCTTAAAAATGTGAGTATTTAAAATTTATCA | RTT |
| | C (SEQ ID NO: 2937) | |
| 2938 | GTTTGCTTAAAAATGTGAGTATTTAAAATTTATC | RTT |
| | AC (SEQ ID NO: 2938) | |
| 2939 | GGTTTGCTTAAAAATGTGAGTATTTAAAATTTAT | RTT |
| | CAC (SEQ ID NO: 2939) | |
| 2940 | TGGTTTGCTTAAAAATGTGAGTATTTAAAATTTA | RTT |
| | TCAC (SEQ ID NO: 2940) | |
| 2941 | CTGGTTTGCTTAAAAATGTGAGTATTTAAAATTT | RTT |
| | ATCAC (SEQ ID NO: 2941) | |
| 2942 | CCTGGTTTGCTTAAAAATGTGAGTATTTAAAATT | RTT |
| | TATCAC (SEQ ID NO: 2942) | |
| 2943 | AAGAAGTTGCTCTTTTCACT (SEQ ID NO: 2943) | PE3 ngRNA spacer (CAAGGT PAM) |
| 2533 | AAGGTCTTGGGTATGCACCT (SEQ ID NO: 2533) | PE3 ngRNA spacer (ATAGAT PAM) |
| 2944 | ATATACTTTCAGCACTCAGA (SEQ ID NO: 2944) | PE3 ngRNA spacer (TTTGAT PAM) |
| 2945 | ATCTCATATACTTTCAGCAC (SEQ ID NO: 2945) | PE3 ngRNA spacer (TCAGAT PAM) |
| 2677 | ATGCACCTATAGATTACTAT (SEQ ID NO: 2677) | PE3 ngRNA spacer (CCTGGT PAM) |
| 2771 | ATTACTATCCTGGTTTGCTT (SEQ ID NO: 2771) | PE3 ngRNA spacer (AAAAAT PAM) |
| 2626 | TATTTAAAATTTATCACTTT (SEQ ID NO: 2626) | PE3 ngRNA spacer (TGAAAT PAM) |
| 2627 | TCAGCACTCAGATTTGATAA (SEQ ID NO: 2627) | PE3 ngRNA spacer (AGAAGT PAM) |
| 2946 | TGCTCTTTTCACTCAAGGTC (SEQ ID NO: 2946) | PE3 ngRNA spacer (TTGGGT PAM) |
| 2947 | TGCTTAAAAATGTGAGTATT (SEQ ID NO: 2947) | PE3b ngRNA spacer (TAAAAT PAM) |

TABLE 42

| Sequence | | |
|---|---|---|
| Number | Sequence | Description |
| 2948 | TCAGCATTAAACATTTC (SEQ ID NO: 2948) | PEgRNA spacer (AAAAGT PAM) |
| 2949 | TTCAGCATTAAACATTTC (SEQ ID NO: 2949) | PEgRNA spacer (AAAAGT PAM) |
| 2950 | ATTCAGCATTAAACATTTC (SEQ ID NO: 2950) | PEgRNA spacer (AAAAGT PAM) |
| 2564 | CATTCAGCATTAAACATTTC (SEQ ID NO: 2564) | PEgRNA spacer (AAAAGT PAM) |
| 2951 | ACATTCAGCATTAAACATTTC (SEQ ID NO: 2951) | PEgRNA spacer (AAAAGT PAM) |
| 2952 | CACATTCAGCATTAAACATTTC (SEQ ID NO: 2952) | PEgRNA spacer (AAAAGT PAM) |
| 2953 | ATGTT | PBS |
| 2954 | ATGTTT | PBS |
| 2955 | ATGTTTA | PBS |
| 2956 | ATGTTTAA | PBS |
| 2957 | ATGTTTAAT | PBS |
| 2958 | ATGTTTAATG (SEQ ID NO: 2958) | PBS |
| 2959 | ATGTTTAATGC (SEQ ID NO: 2959) | PBS |
| 2960 | ATGTTTAATGCT (SEQ ID NO: 2960) | PBS |
| 2961 | ATGTTTAATGCTG (SEQ ID NO: 2961) | PBS |
| 2962 | ATGTTTAATGCTGA (SEQ ID NO: 2962) | PBS |
| 2963 | ATGTTTAATGCTGAA (SEQ ID NO: 2963) | PBS |
| 2964 | ATGTTTAATGCTGAAT (SEQ ID NO: 2964) | PBS |
| 2965 | ATGTTTAATGCTGAATG (SEQ ID NO: 2965) | PBS |
| 2966 | ATGTTTAATGCTGAATGT (SEQ ID NO: 2966) | PBS |
| 2967 | ATGTTTAATGCTGAATGTG (SEQ ID NO: 2967) | PBS |
| 2968 | AGTATTTAAAATTTATCACTTTTGAA (SEQ ID NO: | RTT |
| | 2968) | |
| 2969 | GAGTATTTAAAATTTATCACTTTTGAA (SEQ ID | RTT |
| | NO: 2969) | |
| 2970 | TGAGTATTTAAAATTTATCACTTTTGAA (SEQ ID | RTT |
| | NO: 2970) | |
| 2971 | GTGAGTATTTAAAATTTATCACTTTTGAA (SEQ ID | RTT |
| | NO: 2971) | |
| 2972 | TGTGAGTATTTAAAATTTATCACTTTTGAA (SEQ | RTT |
| | ID NO: 2972) | |
| 2973 | ATGTGAGTATTTAAAATTTATCACTTTTGAA (SEQ | RTT |
| | ID NO: 2973) | |
| 2974 | AATGTGAGTATTTAAAATTTATCACTTTTGAA | RTT |
| | (SEQ ID NO: 2974) | |
| 2975 | AAATGTGAGTATTTAAAATTTATCACTTTTGAA | RTT |
| | (SEQ ID NO: 2975) | |
| 2976 | AAAATGTGAGTATTTAAAATTTATCACTTTTGAA | RTT |
| | (SEQ ID NO: 2976) | |
| 2977 | AAAAATGTGAGTATTTAAAATTTATCACTTTTGA | RTT |
| | A (SEQ ID NO: 2977) | |
| 2978 | TAAAAATGTGAGTATTTAAAATTTATCACTTTTG | RTT |
| | AA (SEQ ID NO: 2978) | |
| 2979 | TTAAAAATGTGAGTATTTAAAATTTATCACTTTT | RTT |
| | GAA (SEQ ID NO: 2979) | |
| 2980 | CTTAAAAATGTGAGTATTTAAAATTTATCACTTT | RTT |
| | TGAA (SEQ ID NO: 2980) | |
| 2981 | GCTTAAAAATGTGAGTATTTAAAATTTATCACTT | RTT |
| | TTGAA (SEQ ID NO: 2981) | |
| 2982 | TGCTTAAAAATGTGAGTATTTAAAATTTATCACT | RTT |
| | TTTGAA (SEQ ID NO: 2982) | |
| 2943 | AAGAAGTTGCTCTTTTCACT (SEQ ID NO: 2943) | PE3 ngRNA spacer (CAAGGT PAM) |
| 2533 | AAGGTCTTGGGTATGCACCT (SEQ ID NO: 2533) | PE3 ngRNA spacer (ATAGAT PAM) |
| 2983 | AATTTATCACTTTTGAAATG (SEQ ID NO: 2983) | PE3 ngRNA spacer (TTTAAT PAM) |
| 2944 | ATATACTTTCAGCACTCAGA (SEQ ID NO: 2944) | PE3 ngRNA spacer (TTTGAT PAM) |
| 2945 | ATCTCATATACTTTCAGCAC (SEQ ID NO: 2945) | PE3 ngRNA spacer (TCAGAT PAM) |
| 2677 | ATGCACCTATAGATTACTAT (SEQ ID NO: 2677) | PE3 ngRNA spacer (CCTGGT PAM) |
| 2771 | ATTACTATCCTGGTTTGCTT (SEQ ID NO: 2771) | PE3 ngRNA spacer (AAAAAT PAM) |
| 2626 | TATTTAAAATTTATCACTTT (SEQ ID NO: 2626) | PE3 ngRNA spacer (TGAAAT PAM) |
| 2627 | TCAGCACTCAGATTTGATAA (SEQ ID NO: 2627) | PE3 ngRNA spacer (AGAAGT PAM) |
| 2946 | TGCTCTTTTCACTCAAGGTC (SEQ ID NO: 2946) | PE3 ngRNA spacer (TTGGGT PAM) |
| 2947 | TGCTTAAAAATGTGAGTATT (SEQ ID NO: 2947) | PE3b ngRNA spacer (TAAAAT PAM) |

TABLE 43

| Sequence | | |
|---|---|---|
| Number | Sequence | Description |
| 2984 | TTCAAAAGTGATAAATT (SEQ ID NO: 2984) | PEgRNA spacer (TTAAAT PAM) |
| 2985 | TTTCAAAAGTGATAAATT (SEQ ID NO: 2985) | PEgRNA spacer (TTAAAT PAM) |
| 2986 | ATTTCAAAAGTGATAAATT (SEQ ID NO: 2986) | PEgRNA spacer (TTAAAT PAM) |
| 2565 | CATTTCAAAAGTGATAAATT (SEQ ID NO: 2565) | PEgRNA spacer (TTAAAT PAM) |
| 2987 | ACATTTCAAAAGTGATAAATT (SEQ ID NO: 2987) | PERNA spacer (TTAAAT PAM) |
| 2988 | AACATTTCAAAAGTGATAAATT (SEQ ID NO: | PEgRNA spacer (TTAAAT PAM) |
| | 2988) | |
| 2989 | TTATC | PBS |
| 2990 | TTATCA | PBS |
| 2991 | TTATCAC | PBS |
| 2992 | TTATCACT | PBS |
| 2993 | TTATCACTT | PBS |
| 2994 | TTATCACTTT (SEQ ID NO: 2994) | PBS |
| 2995 | TTATCACTTTT (SEQ ID NO: 2995) | PBS |
| 2996 | TTATCACTTTTG (SEQ ID NO: 2996) | PBS |
| 2997 | TTATCACTTTTGA (SEQ ID NO: 2997) | PBS |
| 2998 | TTATCACTTTTGAA (SEQ ID NO: 2998) | PBS |
| 2999 | TTATCACTTTTGAAA (SEQ ID NO: 2999) | PBS |
| 3000 | TTATCACTTTTGAAAT (SEQ ID NO: 3000) | PBS |
| 3001 | TTATCACTTTTGAAATG (SEQ ID NO: 3001) | PBS |
| 3002 | TTATCACTTTTGAAATGT (SEQ ID NO: 3002) | PBS |
| 3003 | TTATCACTTTTGAAATGTT (SEQ ID NO: 3003) | PBS |
| 3004 | AGTATTTAAAAT (SEQ ID NO: 3004) | RTT |
| 3005 | GAGTATTTAAAAT (SEQ ID NO: 3005) | RTT |
| 3006 | TGAGTATTTAAAAT (SEQ ID NO: 3006) | RTT |
| 3007 | GTGAGTATTTAAAAT (SEQ ID NO: 3007) | RTT |
| 3008 | TGTGAGTATTTAAAAT (SEQ ID NO: 3008) | RTT |
| 3009 | ATGTGAGTATTTAAAAT (SEQ ID NO: 3009) | RTT |
| 3010 | AATGTGAGTATTTAAAAT (SEQ ID NO: 3010) | RTT |
| 3011 | AAATGTGAGTATTTAAAAT (SEQ ID NO: 3011) | RTT |
| 3012 | AAAATGTGAGTATTTAAAAT (SEQ ID NO: 3012) | RTT |
| 3013 | AAAAATGTGAGTATTTAAAAT (SEQ ID NO: 3013) | RTT |
| 3014 | TAAAAATGTGAGTATTTAAAAT (SEQ ID NO: | RTT |
| | 3014) | |
| 3015 | TTAAAAATGTGAGTATTTAAAAT (SEQ ID NO: | RTT |
| | 3015) | |
| 3016 | CTTAAAAATGTGAGTATTTAAAAT (SEQ ID NO: | RTT |
| | 3016) | |
| 3017 | GCTTAAAAATGTGAGTATTTAAAAT (SEQ ID NO: | RTT |
| | 3017) | |
| 3018 | TGCTTAAAAATGTGAGTATTTAAAAT (SEQ ID | RTT |
| | NO: 3018) | |
| 3019 | TTGCTTAAAAATGTGAGTATTTAAAAT (SEQ ID | RTT |
| | NO: 3019) | |
| 3020 | TTTGCTTAAAAATGTGAGTATTTAAAAT (SEQ ID | RTT |
| | NO: 3020) | |
| 3021 | GTTTGCTTAAAAATGTGAGTATTTAAAAT (SEQ | RTT |
| | ID NO: 3021) | |
| 3022 | GGTTTGCTTAAAAATGTGAGTATTTAAAAT (SEQ | RTT |
| | ID NO: 3022) | |
| 3023 | TGGTTTGCTTAAAAATGTGAGTATTTAAAAT | RTT |
| | (SEQ ID NO: 3023) | |
| 3024 | CTGGTTTGCTTAAAAATGTGAGTATTTAAAAT | RTT |
| | (SEQ ID NO: 3024) | |
| 3025 | CCTGGTTTGCTTAAAAATGTGAGTATTTAAAAT | RTT |
| | (SEQ ID NO: 3025) | |
| 3026 | TCCTGGTTTGCTTAAAAATGTGAGTATTTAAAAT | RTT |
| | (SEQ ID NO: 3026) | |
| 3027 | ATCCTGGTTTGCTTAAAAATGTGAGTATTTAAAA | RTT |
| | T (SEQ ID NO: 3027) | |
| 3028 | TATCCTGGTTTGCTTAAAAATGTGAGTATTTAAA | RTT |
| | AT (SEQ ID NO: 3028) | |
| 3029 | CTATCCTGGTTTGCTTAAAAATGTGAGTATTTAA | RTT |
| | AAT (SEQ ID NO: 3029) | |
| 3030 | ACTATCCTGGTTTGCTTAAAAATGTGAGTATTTA | RTT |
| | AAAT (SEQ ID NO: 3030) | |
| 3031 | TACTATCCTGGTTTGCTTAAAAATGTGAGTATTT | RTT |
| | AAAAT (SEQ ID NO: 3031) | |
| 3032 | TTACTATCCTGGTTTGCTTAAAAATGTGAGTATTT | RTT |
| | AAAAT (SEQ ID NO: 3032) | |
| 2943 | AAGAAGTTGCTCTTTTCACT (SEQ ID NO: 2943) | PE3 ngRNA spacer (CAAGGT PAM) |
| 2533 | AAGGTCTTGGGTATGCACCT (SEQ ID NO: 2533) | PE3 ngRNA spacer (ATAGAT PAM) |
| 2944 | ATATACTTTCAGCACTCAGA (SEQ ID NO: 2944) | PE3 ngRNA spacer (TTTGAT PAM) |
| 2945 | ATCTCATATACTTTCAGCAC (SEQ ID NO: 2945) | PE3 ngRNA spacer (TCAGAT PAM) |
| 2677 | ATGCACCTATAGATTACTAT (SEQ ID NO: 2677) | PE3 ngRNA spacer (CCTGGT PAM) |
| 2771 | ATTACTATCCTGGTTTGCTT (SEQ ID NO: 2771) | PE3 ngRNA spacer (AAAAAT PAM) |
| 2627 | TCAGCACTCAGATTTGATAA (SEQ ID NO: 2627) | PE3 ngRNA spacer (AGAAGT PAM) |
| 2946 | TGCTCTTTTCACTCAAGGTC (SEQ ID NO: 2946) | PE3 ngRNA spacer (TTGGGT PAM) |
| 2947 | TGCTTAAAAATGTGAGTATT (SEQ ID NO: 2947) | PE3b ngRNA spacer (TAAAAT PAM) |

TABLE 44

| Sequence | | |
|---|---|---|
| Number | Sequence | Description |
| 3033 | GTTTGCTTAAAAATGTG (SEQ ID NO: 3033) | PERNA spacer (TG PAM) |
| 3034 | GGTTTGCTTAAAAATGTG (SEQ ID NO: 3034) | PEgRNA spacer (TG PAM) |
| 3035 | TGGTTTGCTTAAAAATGTG (SEQ ID NO: 3035) | PEgRNA spacer (TG PAM) |
| 2617 | CTGGTTTGCTTAAAAATGTG (SEQ ID NO: 2617) | PEgRNA spacer (TG PAM) |
| 3036 | CCTGGTTTGCTTAAAAATGTG (SEQ ID NO: 3036) | PEgRNA spacer (TG PAM) |
| 3037 | TCCTGGTTTGCTTAAAAATGTG (SEQ ID NO: 3037) | PEgRNA spacer (TG PAM) |
| 3038 | ATTTT | PBS |
| 3039 | ATTTTT | PBS |
| 3040 | ATTTTTA | PBS |
| 3041 | ATTTTTAA | PBS |
| 3042 | ATTTTTAAG | PBS |
| 3043 | ATTTTTAAGC (SEQ ID NO: 3043) | PBS |
| 3044 | ATTTTTAAGCA (SEQ ID NO: 3044) | PBS |
| 3045 | ATTTTTAAGCAA (SEQ ID NO: 3045) | PBS |
| 3046 | ATTTTTAAGCAAA (SEQ ID NO: 3046) | PBS |
| 3047 | ATTTTTAAGCAAAC (SEQ ID NO: 3047) | PBS |
| 3048 | ATTTTTAAGCAAACC (SEQ ID NO: 3048) | PBS |
| 3049 | ATTTTTAAGCAAACCA (SEQ ID NO: 3049) | PBS |
| 3050 | ATTTTTAAGCAAACCAG (SEQ ID NO: 3050) | PBS |
| 3051 | ATTTTTAAGCAAACCAGG (SEQ ID NO: 3051) | PBS |
| 3052 | ATTTTTAAGCAAACCAGGA (SEQ ID NO: 3052) | PBS |
| 3053 | AAATACTCAC (SEQ ID NO: 3053) | RTT |
| 3054 | TAAATACTCAC (SEQ ID NO: 3054) | RTT |
| 3055 | TTAAATACTCAC (SEQ ID NO: 3055) | RTT |
| 3056 | TTTAAATACTCAC (SEQ ID NO: 3056) | RTT |
| 3057 | TTTTAAATACTCAC (SEQ ID NO: 3057) | RTT |
| 3058 | ATTTTAAATACTCAC (SEQ ID NO: 3058) | RTT |
| 3059 | AATTTTAAATACTCAC (SEQ ID NO: 3059) | RTT |
| 3060 | AAATTTTAAATACTCAC (SEQ ID NO: 3060) | RTT |
| 3061 | TAAATTTTAAATACTCAC (SEQ ID NO: 3061) | RTT |
| 3062 | ATAAATTTTAAATACTCAC (SEQ ID NO: 3062) | RTT |
| 3063 | GATAAATTTTAAATACTCAC (SEQ ID NO: 3063) | RTT |
| 3064 | TGATAAATTTTAAATACTCAC (SEQ ID NO: 3064) | RTT |
| 3065 | GTGATAAATTTTAAATACTCAC (SEQ ID NO: 3065) | RTT |
| 3066 | AGTGATAAATTTTAAATACTCAC (SEQ ID NO: | RTT |
| | 3066) | |
| 3067 | AAGTGATAAATTTTAAATACTCAC (SEQ ID NO: | RTT |
| | 3067) | |
| 3068 | AAAGTGATAAATTTTAAATACTCAC (SEQ ID NO: | RTT |
| | 3068) | |
| 3069 | AAAAGTGATAAATTTTAAATACTCAC (SEQ ID | RTT |
| | NO: 3069) | |
| 3070 | CAAAAGTGATAAATTTTAAATACTCAC (SEQ ID | RTT |
| | NO: 3070) | |
| 3071 | TCAAAAGTGATAAATTTTAAATACTCAC (SEQ ID | RTT |
| | NO: 3071) | |
| 3072 | TTCAAAAGTGATAAATTTTAAATACTCAC (SEQ | RTT |
| | ID NO: 3072) | |
| 3073 | TTTCAAAAGTGATAAATTTTAAATACTCAC (SEQ | RTT |
| | ID NO: 3073) | |
| 3074 | ATTTCAAAAGTGATAAATTTTAAATACTCAC | RTT |
| | (SEQ ID NO: 3074) | |
| 3075 | CATTTCAAAAGTGATAAATTTTAAATACTCAC | RTT |
| | (SEQ ID NO: 3075) | |
| 3076 | ACATTTCAAAAGTGATAAATTTTAAATACTCAC | RTT |
| | (SEQ ID NO: 3076) | |
| 3077 | AACATTTCAAAAGTGATAAATTTTAAATACTCAC | RTT |
| | (SEQ ID NO: 3077) | |
| 3078 | AAACATTTCAAAAGTGATAAATTTTAAATACTCA | RTT |
| | C (SEQ ID NO: 3078) | |
| 3079 | TAAACATTTCAAAAGTGATAAATTTTAAATACTC | RTT |
| | AC (SEQ ID NO: 3079) | |
| 3080 | TTAAACATTTCAAAAGTGATAAATTTTAAATACT | RTT |
| | CAC (SEQ ID NO: 3080) | |
| 3081 | ATTAAACATTTCAAAAGTGATAAATTTTAAATAC | RTT |
| | TCAC (SEQ ID NO: 3081) | |
| 3082 | CATTAAACATTTCAAAAGTGATAAATTTTAAATA | RTT |
| | CTCAC (SEQ ID NO: 3082) | |
| 3083 | GCATTAAACATTTCAAAAGTGATAAATTTTAAAT | RTT |
| | ACTCAC (SEQ ID NO: 3083) | |
| 2885 | AAAAGTTAAACATCTCTTCT (SEQ ID NO: 2885) | PE3 ngRNA spacer (GG PAM) |
| 2559 | AAACATCTCTTCTGGAGGAC (SEQ ID NO: 2559) | PE3 ngRNA spacer (TG PAM) |
| 2886 | AAGTTAAACATCTCTTCTGG (SEQ ID NO: 2886) | PE3 ngRNA spacer (AG PAM) |
| 3084 | AGAAGAGATAAACAAAGAAA (SEQ ID NO: 3084) | PE3 ngRNA spacer (AG PAM) |
| 2573 | AGCATTAAACATTTCAAAAG (SEQ ID NO: 2573) | PE3 ngRNA spacer (TG PAM) |
| 3085 | AGGTAAGAAGAGATAAACAA (SEQ ID NO: 3085) | PE3 ngRNA spacer (AG PAM) |
| 2887 | AGTTAAACATCTCTTCTGGA (SEQ ID NO: 2887) | PE3 ngRNA spacer (GG PAM) |
| 2888 | ATATTTCCATTTACTCTTTT (SEQ ID NO: 2888) | PE3 ngRNA spacer (TG PAM) |
| 2889 | CATTTACTCTTTTTGCTGAT (SEQ ID NO: 2889) | PE3 ngRNA spacer (GG PAM) |
| 2890 | CCATTTACTCTTTTTGCTGA (SEQ ID NO: 2890) | PE3 ngRNA spacer (TG PAM) |
| 2892 | GAAAAGTTAAACATCTCTTC (SEQ ID NO: 2892) | PE3 ngRNA spacer (TG PAM) |
| 2569 | TCAGCATTAAACATTTCAAA (SEQ ID NO: 2569) | PE3 ngRNA spacer (AG PAM) |
| 2895 | TTTCCATTTACTCTTTTTGC (SEQ ID NO: 2895) | PE3 ngRNA spacer (TG PAM) |
| 2896 | TTTTTGCTGATGGCACATTC (SEQ ID NO: 2896) | PE3 ngRNA spacer (AG PAM) |
| 2897 | AAGTTAAACATCTCTTCTGGGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2898 | GAAAAGTTAAACATCTCTTCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 2898) | |
| 2899 | AAGTTAAACATCTCTTCTGGGTTTTAGAGCTAGA | PE3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |
| 2900 | GAAAAGTTAAACATCTCTTCGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2900) | |

TABLE 45

| Sequence | | |
|---|---|---|
| Number | Sequence | Description |
| 3086 | CCTATAGATTACTATCC (SEQ ID NO: 3086) | PEgRNA spacer (TG; TGG PAM) |
| 3087 | ACCTATAGATTACTATCC (SEQ ID NO: 3087) | PEgRNA spacer (TG; TGG PAM) |
| 3088 | CACCTATAGATTACTATCC (SEQ ID NO: 3088) | PEgRNA spacer (TG; TGG PAM) |
| 2619 | GCACCTATAGATTACTATCC (SEQ ID NO: 2619) | PEgRNA spacer (TG; TGG PAM) |
| 3089 | TGCACCTATAGATTACTATCC (SEQ ID NO: 3089) | PEgRNA spacer (TG; TGG PAM) |
| 3090 | ATGCACCTATAGATTACTATCC (SEQ ID NO: 3090) | PEgRNA spacer (TG; TGG PAM) |
| 3091 | TAGTA | PBS |
| 3092 | TAGTAA | PBS |
| 3093 | TAGTAAT | PBS |
| 3094 | TAGTAATC | PBS |
| 3095 | TAGTAATCT | PBS |
| 3096 | TAGTAATCTA (SEQ ID NO: 3096) | PBS |
| 3097 | TAGTAATCTAT (SEQ ID NO: 3097) | PBS |
| 3098 | TAGTAATCTATA (SEQ ID NO: 3098) | PBS |
| 3099 | TAGTAATCTATAG (SEQ ID NO: 3099) | PBS |
| 3100 | TAGTAATCTATAGG (SEQ ID NO: 3100) | PBS |
| 3101 | TAGTAATCTATAGGT (SEQ ID NO: 3101) | PBS |
| 3102 | TAGTAATCTATAGGTG (SEQ ID NO: 3102) | PBS |
| 3103 | TAGTAATCTATAGGTGC (SEQ ID NO: 3103) | PBS |
| 3104 | TAGTAATCTATAGGTGCA (SEQ ID NO: 3104) | PBS |
| 3105 | TAGTAATCTATAGGTGCAT (SEQ ID NO: 3105) | PBS |
| 3106 | TCACATTTTTAAGCAAACCAGGA (SEQ ID NO: | RTT |
| | 3106) | |
| 3107 | CTCACATTTTTAAGCAAACCAGGA (SEQ ID NO: | RTT |
| | 3107) | |
| 3108 | ACTCACATTTTTAAGCAAACCAGGA (SEQ ID NO: | RTT |
| | 3108) | |
| 3109 | TACTCACATTTTTAAGCAAACCAGGA (SEQ ID | RTT |
| | NO: 3109) | |
| 3110 | ATACTCACATTTTTAAGCAAACCAGGA (SEQ ID | RTT |
| | NO: 3110) | |
| 3111 | AATACTCACATTTTTAAGCAAACCAGGA (SEQ ID | RTT |
| | NO: 3111) | |
| 3112 | AAATACTCACATTTTTAAGCAAACCAGGA (SEQ | RTT |
| | ID NO: 3112) | |
| 3113 | TAAATACTCACATTTTTAAGCAAACCAGGA (SEQ | RTT |
| | ID NO: 3113) | |
| 3114 | TTAAATACTCACATTTTTAAGCAAACCAGGA | RTT |
| | (SEQ ID NO: 3114) | |
| 3115 | TTTAAATACTCACATTTTTAAGCAAACCAGGA | RTT |
| | (SEQ ID NO: 3115) | |
| 3116 | TTTTAAATACTCACATTTTTAAGCAAACCAGGA | RTT |
| | (SEQ ID NO: 3116) | |
| 3117 | ATTTTAAATACTCACATTTTTAAGCAAACCAGGA | RTT |
| | (SEQ ID NO: 3117) | |
| 3118 | AATTTTAAATACTCACATTTTTAAGCAAACCAGG | RTT |
| | A (SEQ ID NO: 3118) | |
| 3119 | AAATTTTAAATACTCACATTTTTAAGCAAACCAG | RTT |
| | GA (SEQ ID NO: 3119) | |
| 3120 | TAAATTTTAAATACTCACATTTTTAAGCAAACCA | RTT |
| | GGA (SEQ ID NO: 3120) | |
| 3121 | ATAAATTTTAAATACTCACATTTTTAAGCAAACC | RTT |
| | AGGA (SEQ ID NO: 3121) | |
| 3122 | GATAAATTTTAAATACTCACATTTTTAAGCAAAC | RTT |
| | CAGGA (SEQ ID NO: 3122) | |
| 3123 | TGATAAATTTTAAATACTCACATTTTTAAGCAAA | RTT |
| | CCAGGA (SEQ ID NO: 3123) | |
| 2885 | AAAAGTTAAACATCTCTTCT (SEQ ID NO: 2885) | PE3 ngRNA spacer (GG PAM) |
| 2559 | AAACATCTCTTCTGGAGGAC (SEQ ID NO: 2559) | PE3 ngRNA spacer (TG PAM) |
| 2886 | AAGTTAAACATCTCTTCTGG (SEQ ID NO: 2886) | PE3 ngRNA spacer (AG; AGG PAM) |
| 2573 | AGCATTAAACATTTCAAAAG (SEQ ID NO: 2573) | PE3 ngRNA spacer (TG PAM) |
| 2887 | AGTTAAACATCTCTTCTGGA (SEQ ID NO: 2887) | PE3 ngRNA spacer (GG PAM) |
| 2888 | ATATTTCCATTTACTCTTTT (SEQ ID NO: 2888) | PE3 ngRNA spacer (TG PAM) |
| 2561 | ATTTTTAAGCAAACCAGGAT (SEQ ID NO: 2561) | PE3 ngRNA spacer (AG PAM) |
| 2889 | CATTTACTCTTTTTGCTGAT (SEQ ID NO: 2889) | PE3 ngRNA spacer (GG PAM) |
| 2890 | CCATTTACTCTTTTTGCTGA (SEQ ID NO: 2890) | PE3 ngRNA spacer (TG; TGG PAM) |
| 2891 | CTCACATTTTTAAGCAAACC (SEQ ID NO: 2891) | PE3b ngRNA spacer (AG; AGG PAM) |
| 2892 | GAAAAGTTAAACATCTCTTC (SEQ ID NO: 2892) | PE3 ngRNA spacer (TG; TGG PAM) |
| 2893 | TCACATTTTTAAGCAAACCA (SEQ ID NO: 2893) | PE3b ngRNA spacer (GG PAM) |
| 2569 | TCAGCATTAAACATTTCAAA (SEQ ID NO: 2569) | PE3 ngRNA spacer (AG PAM) |
| 2894 | TTTAAATACTCACATTTTTA (SEQ ID NO: 2894) | PE3b ngRNA spacer (AG PAM) |
| 2895 | TTTCCATTTACTCTTTTTGC (SEQ ID NO: 2895) | PE3 ngRNA spacer (TG PAM) |
| 2896 | TTTTTGCTGATGGCACATTC (SEQ ID NO: 2896) | PE3 ngRNA spacer (AG PAM) |
| 3124 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATC (SEQ | |
| | ID NO: 3124) | |
| 3125 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTA | |
| | (SEQ ID NO: 3125) | |
| 3126 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATC | |
| | (SEQ ID NO: 3126) | |
| 3127 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTTTT | |
| | (SEQ ID NO: 3127) | |
| 3128 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTATA | |
| | (SEQ ID NO: 3128) | |
| 3129 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTA | |
| | (SEQ ID NO: 3129) | |
| 3130 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | (SEQ ID NO: 3130) | |
| 3131 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTATT | |
| | TT (SEQ ID NO: 3131) | |
| 3132 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTT | |
| | TT (SEQ ID NO: 3132) | |
| 3133 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTATA | |
| | GG (SEQ ID NO: 3133) | |
| 3134 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTA | |
| | TA (SEQ ID NO: 3134) | |
| 3135 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TA (SEQ ID NO: 3135) | |
| 3136 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | C (SEQ ID NO: 3136) | |
| 3137 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTATA | |
| | TTTT (SEQ ID NO: 3137) | |
| 3138 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTA | |
| | TTTT (SEQ ID NO: 3138) | |
| 3139 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TTTT (SEQ ID NO: 3139) | |
| 3140 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTA | |
| | TAGG (SEQ ID NO: 3140) | |
| 3141 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TATA (SEQ ID NO: 3141) | |
| 3142 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTA (SEQ ID NO: 3142) | |
| 3143 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCATACT | |
| | CACATTTTTAAGCAAACCAGGATAGTAATCTATA | |
| | GGTTTT (SEQ ID NO: 3143) | |
| 3144 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTA | |
| | TATTTT (SEQ ID NO: 3144) | |
| 3145 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TATTTT (SEQ ID NO: 3145) | |
| 3146 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTTTT (SEQ ID NO: 3146) | |
| 3147 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TATAGG (SEQ ID NO: 3147) | |
| 3148 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTATA (SEQ ID NO: 3148) | |
| 3149 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCAAATA | |
| | CTCACATTTTTAAGCAAACCAGGATAGTAATCTA | |
| | TAGGTTTT (SEQ ID NO: 3149) | |
| 3150 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TATATTTT (SEQ ID NO: 3150) | |
| 3151 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTATTTT (SEQ ID NO: 3151) | |
| 3152 | GCACCTATAGATTACTATCCGTTTTAGAGCTAGA | pegRNA |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTATAGG (SEQ ID NO: 3152) | |
| 3153 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTAAA | |
| | TACTCACATTTTTAAGCAAACCAGGATAGTAATC | |
| | TATAGGTTTT (SEQ ID NO: 3153) | |
| 3154 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTATATTTT (SEQ ID NO: 3154) | |
| 3155 | GCACCTATAGATTACTATCCGTTTTAGAGCTAGA | pegRNA, contains end adaptations |
| | AATAGCAAGTTAAAATAAGGCTAGTCCGTTATCA | |
| | ACTTGAAAAAGTGGCACCGAGTCGGTGCTTTTAA | |
| | ATACTCACATTTTTAAGCAAACCAGGATAGTAAT | |
| | CTATAGGTTTT (SEQ ID NO: 3155) | |
| 2897 | AAGTTAAACATCTCTTCTGGGTTTTAGAGCTAGA | PE3 ngRNA (AG; AGG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2898 | GAAAAGTTAAACATCTCTTCGTTTTAGAGCTAGA | PE3 ngRNA (TG; TGG PAM) |
| | AATAGCAAGTTAAAATAAGGCTAGTCCGTTATCA | |
| | ACTTGAAAAAGTGGCACCGAGTCGGTGC (SEQ ID | |
| | NO: 2898) | |
| 2899 | AAGTTAAACATCTCTTCTGGGTTTTAGAGCTAGA | PE3 ngRNA (AG; AGG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |
| 2900 | GAAAAGTTAAACATCTCTTCGTTTTAGAGCTAGA | PE3 ngRNA (TG; TGG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2900) | |

TABLE 46

| Sequence | | |
|---|---|---|
| Number | Sequence | Description |
| 3156 | CTTGGGTATGCACCTAT (SEQ ID NO: 3156) | PEgRNA spacer (AG;AGA PAM) |
| 3157 | TCTTGGGTATGCACCTAT (SEQ ID NO: 3157) | PEgRNA spacer (AG;AGA PAM) |
| 3158 | GTCTTGGGTATGCACCTAT (SEQ ID NO: 3158) | PEgRNA spacer (AG;AGA PAM) |
| 2622 | GGTCTTGGGTATGCACCTAT (SEQ ID NO: 2622) | PEgRNA spacer (AG;AGA PAM) |
| 3159 | AGGTCTTGGGTATGCACCTAT (SEQ ID NO: 3159) | PEgRNA spacer (AG;AGA PAM) |
| 3160 | AAGGTCTTGGGTATGCACCTAT (SEQ ID NO: 3160) | PEgRNA spacer (AG;AGA PAM) |
| 3161 | GGTGC | PBS |
| 3162 | GGTGCA | PBS |
| 3163 | GGTGCAT | PBS |
| 3164 | GGTGCATA | PBS |
| 3165 | GGTGCATAC | PBS |
| 3166 | GGTGCATACC (SEQ ID NO: 3166) | PBS |
| 3167 | GGTGCATACCC (SEQ ID NO: 3167) | PBS |
| 3168 | GGTGCATACCCA (SEQ ID NO: 3168) | PBS |
| 3169 | GGTGCATACCCAA (SEQ ID NO: 3169) | PBS |
| 3170 | GGTGCATACCCAAG (SEQ ID NO: 3170) | PBS |
| 3171 | GGTGCATACCCAAGA (SEQ ID NO: 3171) | PBS |
| 3172 | GGTGCATACCCAAGAC (SEQ ID NO: 3172) | PBS |
| 3173 | GGTGCATACCCAAGACC (SEQ ID NO: 3173) | PBS |
| 3174 | GGTGCATACCCAAGACCT (SEQ ID NO: 3174) | PBS |
| 3175 | GGTGCATACCCAAGACCTT (SEQ ID NO: 3175) | PBS |
| 3176 | TCACATTTTTAAGCAAACCAGGATAGTAATCTAT | RTT |
| | A (SEQ ID NO: 3176) | |
| 3177 | CTCACATTTTTAAGCAAACCAGGATAGTAATCTA | RTT |
| | TA (SEQ ID NO: 3177) | |
| 3178 | ACTCACATTTTTAAGCAAACCAGGATAGTAATCT | RTT |
| | ATA (SEQ ID NO: 3178) | |
| 3179 | TACTCACATTTTTAAGCAAACCAGGATAGTAATC | RTT |
| | TATA (SEQ ID NO: 3179) | |
| 3180 | ATACTCACATTTTTAAGCAAACCAGGATAGTAAT | RTT |
| | CTATA (SEQ ID NO: 3180) | |
| 3181 | AATACTCACATTTTTAAGCAAACCAGGATAGTAA | RTT |
| | TCTATA (SEQ ID NO: 3181) | |
| 2559 | AAACATCTCTTCTGGAGGAC (SEQ ID NO: 2559) | PE3 ngRNA spacer (TG;TGA PAM) |
| 3182 | AAACCAGGATAGTAATCTAT (SEQ ID NO: 3182) | PE3 ngRNA spacer (AG PAM) |
| 3183 | AACCAGGATAGTAATCTATA (SEQ ID NO: 3183) | PE3 ngRNA spacer (GG PAM) |
| 2886 | AAGTTAAACATCTCTTCTGG (SEQ ID NO: 2886) | PE3 ngRNA spacer (AG PAM) |
| 2573 | AGCATTAAACATTTCAAAAG (SEQ ID NO: 2573) | PE3 ngRNA spacer (TG;TGA PAM) |
| 2887 | AGTTAAACATCTCTTCTGGA (SEQ ID NO: 2887) | PE3 ngRNA spacer (GG;GGA PAM) |
| 2888 | ATATTTCCATTTACTCTTTT (SEQ ID NO: 2888) | PE3 ngRNA spacer (TG PAM) |
| 2561 | ATTTTTAAGCAAACCAGGAT (SEQ ID NO: 2561) | PE3 ngRNA spacer (AG PAM) |
| 2889 | CATTTACTCTTTTTGCTGAT (SEQ ID NO: 2889) | PE3 ngRNA spacer (GG PAM) |
| 3184 | CCAGGATAGTAATCTATAGG (SEQ ID NO: 3184) | PE3 ngRNA spacer (TG PAM) |
| 2890 | CCATTTACTCTTTTTGCTGA (SEQ ID NO: 2890) | PE3 ngRNA spacer (TG PAM) |
| 2891 | CTCACATTTTTAAGCAAACC (SEQ ID NO: 2891) | PE3b ngRNA spacer (AG PAM) |
| 2893 | TCACATTTTTAAGCAAACCA (SEQ ID NO: 2893) | PE3b ngRNA spacer (GG;GGA PAM) |
| 2569 | TCAGCATTAAACATTTCAAA (SEQ ID NO: 2569) | PE3 ngRNA spacer (AG PAM) |
| 2894 | TTTAAATACTCACATTTTTA (SEQ ID NO: 2894) | PE3b ngRNA spacer (AG PAM) |
| 2895 | TTTCCATTTACTCTTTTTGC (SEQ ID NO: 2895) | PE3 ngRNA spacer (TG;TGA PAM) |
| 2896 | TTTTTGCTGATGGCACATTC (SEQ ID NO: 2896) | PE3 ngRNA spacer (AG PAM) |
| 2897 | AAGTTAAACATCTCTTCTGGGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2899 | AAGTTAAACATCTCTTCTGGGTTTTAGAGCTAGA | PE3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |

TABLE 47

| Sequence | | |
|---|---|---|
| Number | Sequence | Description |
| 3185 | AGATTACTATCCTGGTT (SEQ ID NO: 3185) | PEgRNA spacer (TG PAM) |
| 3186 | TAGATTACTATCCTGGTT (SEQ ID NO: 3186) | PEgRNA spacer (TG PAM) |
| 3187 | ATAGATTACTATCCTGGTT (SEQ ID NO: 3187) | PEgRNA spacer (TG PAM) |
| 2624 | TATAGATTACTATCCTGGTT (SEQ ID NO: 2624) | PEgRNA spacer (TG PAM) |
| 3188 | CTATAGATTACTATCCTGGTT (SEQ ID NO: 3188) | PEgRNA spacer (TG PAM) |
| 3189 | CCTATAGATTACTATCCTGGTT (SEQ ID NO: 3189) | PEgRNA spacer (TG PAM) |
| 3190 | CAGGA | PBS |
| 3191 | CAGGAT | PBS |
| 3192 | CAGGATA | PBS |
| 3193 | CAGGATAG | PBS |
| 3194 | CAGGATAGT | PBS |
| 3195 | CAGGATAGTA (SEQ ID NO: 3195) | PBS |
| 3196 | CAGGATAGTAA (SEQ ID NO: 3196) | PBS |
| 3197 | CAGGATAGTAAT (SEQ ID NO: 3197) | PBS |
| 3198 | CAGGATAGTAATC (SEQ ID NO: 3198) | PBS |
| 3199 | CAGGATAGTAATCT (SEQ ID NO: 3199) | PBS |
| 3200 | CAGGATAGTAATCTA (SEQ ID NO: 3200) | PBS |
| 3201 | CAGGATAGTAATCTAT (SEQ ID NO: 3201) | PBS |
| 3202 | CAGGATAGTAATCTATA (SEQ ID NO: 3202) | PBS |
| 3203 | CAGGATAGTAATCTATAG (SEQ ID NO: 3203) | PBS |
| 3204 | CAGGATAGTAATCTATAGG (SEQ ID NO: 3204) | PBS |
| 3205 | TCACATTTTTAAGAAGAC (SEQ ID NO: 3205) | RTT*1,6,11 (TG-to-TT PAM silencing edit) |
| 3206 | TCACATTTTTAAGCAAAC (SEQ ID NO: 3206) | RTT*2,7,11 |
| 3207 | TCACATTTTTAAGGAGAC (SEQ ID NO: 3207) | RTT*3,8,11 (TG-to-TC PAM silencing edit) |
| 3208 | TCACATTTTTAAGTAAAC (SEQ ID NO: 3208) | RTT*4,9,11 (TG-to-TA PAM silencing edit) |
| 3209 | TCACATTTTTAAGTAGAC (SEQ ID NO: 3209) | RTT*5,10,11 (TG-to-TA PAM silencing edit) |
| 3210 | CTCACATTTTTAAGAAGAC (SEQ ID NO: 3210) | RTT*1,6,11 (TG-to-TT PAM silencing edit) |
| 3211 | CTCACATTTTTAAGCAAAC (SEQ ID NO: 3211) | RTT*2,7,11 |
| 3212 | CTCACATTTTTAAGGAGAC (SEQ ID NO: 3212) | RTT*3,8,11 (TG-to-TC PAM silencing edit) |
| 3213 | CTCACATTTTTAAGTAAAC (SEQ ID NO: 3213) | RTT*4,9,11 (TG-to-TA PAM silencing edit) |
| 3214 | CTCACATTTTTAAGTAGAC (SEQ ID NO: 3214) | RTT*5,10,11 (TG-to-TA PAM silencing edit) |
| 3215 | ACTCACATTTTTAAGAAGAC (SEQ ID NO: 3215) | RTT*1,6,11 (TG-to-TT PAM silencing edit) |
| 2560 | ACTCACATTTTTAAGCAAAC (SEQ ID NO: 2560) | RTT*2,7,11 |
| 3216 | ACTCACATTTTTAAGGAGAC (SEQ ID NO: 3216) | RTT*3,8,11 (TG-to-TC PAM silencing edit) |
| 3217 | ACTCACATTTTTAAGTAAAC (SEQ ID NO: 3217) | RTT*4,9,11 (TG-to-TA PAM silencing edit) |
| 3218 | ACTCACATTTTTAAGTAGAC (SEQ ID NO: 3218) | RTT*5,10,11 (TG-to-TA PAM silencing edit) |
| 3219 | TACTCACATTTTTAAGAAGAC (SEQ ID NO: 3219) | RTT*1,6,11 (TG-to-TT PAM silencing edit) |
| 3220 | TACTCACATTTTTAAGCAAAC (SEQ ID NO: 3220) | RTT*2,7,11 |
| 3221 | TACTCACATTTTTAAGGAGAC (SEQ ID NO: 3221) | RTT*3,8,11 (TG-to-TC PAM silencing edit) |
| 3222 | TACTCACATTTTTAAGTAAAC (SEQ ID NO: 3222) | RTT*4,9,11 (TG-to-TA PAM silencing edit) |
| 3223 | TACTCACATTTTTAAGTAGAC (SEQ ID NO: 3223) | RTT*5,10,11 (TG-to-TA PAM silencing edit) |
| 3224 | ATACTCACATTTTTAAGAAGAC (SEQ ID NO: 3224) | RTT*1,6,11 (TG-to-TT PAM silencing edit) |
| 3225 | ATACTCACATTTTTAAGCAAAC (SEQ ID NO: 3225) | RTT*2,7,11 |
| 3226 | ATACTCACATTTTTAAGGAGAC (SEQ ID NO: 3226) | RTT*3,8,11 (TG-to-TC PAM silencing edit) |
| 3227 | ATACTCACATTTTTAAGTAAAC (SEQ ID NO: 3227) | RTT*4,9,11 (TG-to-TA PAM silencing edit) |
| 3228 | ATACTCACATTTTTAAGTAGAC (SEQ ID NO: 3228) | RTT*5,10,11 (TG-to-TA PAM silencing edit) |
| 3229 | AATACTCACATTTTTAAGAAGAC (SEQ ID NO: | RTT*1,6,11 (TG-to-TT PAM silencing edit) |
| | 3229) | |
| 3230 | AATACTCACATTTTTAAGCAAAC (SEQ ID NO: | RTT*2,7,11 |
| | 3230) | |
| 3231 | AATACTCACATTTTTAAGGAGAC (SEQ ID NO: | RTT*3,8,11 (TG-to-TC PAM silencing edit) |
| | 3231) | |
| 3232 | AATACTCACATTTTTAAGTAAAC (SEQ ID NO: | RTT*4,9,11 (TG-to-TA PAM silencing edit) |
| | 3232) | |
| 3233 | AATACTCACATTTTTAAGTAGAC (SEQ ID NO: | RTT*5,10,11 (TG-to-TA PAM silencing edit) |
| | 3233) | |
| 3234 | AAATACTCACATTTTTAAGAAGAC (SEQ ID NO: | RTT*1,6,11 (TG-to-TT PAM silencing edit) |
| | 3234) | |
| 3235 | AAATACTCACATTTTTAAGCAAAC (SEQ ID NO: | RTT*2,7,11 |
| | 3235) | |
| 3236 | AAATACTCACATTTTTAAGGAGAC (SEQ ID NO: | RTT*3,8,11 (TG-to-TC PAM silencing edit) |
| | 3236) | |
| 3237 | AAATACTCACATTTTTAAGTAAAC (SEQ ID NO: | RTT*4,9,11 (TG-to-TA PAM silencing edit) |
| | 3237) | |
| 3238 | AAATACTCACATTTTTAAGTAGAC (SEQ ID NO: | RTT*5,10,11 (TG-to-TA PAM silencing edit) |
| | 3238) | |
| 3239 | TAAATACTCACATTTTTAAGAAGAC (SEQ ID NO: | RTT*1,6,11 (TG-to-TT PAM silencing edit) |
| | 3239) | |
| 3240 | TAAATACTCACATTTTTAAGCAAAC (SEQ ID NO: | RTT*2,7,11 |
| | 3240) | |
| 3241 | TAAATACTCACATTTTTAAGGAGAC (SEQ ID NO: | RTT*3,8,11 (TG-to-TC PAM silencing edit) |
| | 3241) | |
| 3242 | TAAATACTCACATTTTTAAGTAAAC (SEQ ID NO: | RTT*4,9,11 (TG-to-TA PAM silencing edit) |
| | 3242) | |
| 3243 | TAAATACTCACATTTTTAAGTAGAC (SEQ ID NO: | RTT*5,10,11 (TG-to-TA PAM silencing edit) |
| | 3243) | |
| 3244 | TTAAATACTCACATTTTTAAGAAGAC (SEQ ID | RTT*1,6,11 (TG-to-TT PAM silencing edit) |
| | NO: 3244) | |
| 3245 | TTAAATACTCACATTTTTAAGCAAAC (SEQ ID NO: | RTT*2,7,11 |
| | 3245) | |
| 3246 | TTAAATACTCACATTTTTAAGGAGAC (SEQ ID | RTT*3,8,11 (TG-to-TC PAM silencing edit) |
| | NO: 3246) | |
| 3247 | TTAAATACTCACATTTTTAAGTAAAC (SEQ ID NO: | RTT*4,9,11 (TG-to-TA PAM silencing edit) |
| | 3247) | |
| 3248 | TTAAATACTCACATTTTTAAGTAGAC (SEQ ID NO: | RTT*5,10,11 (TG-to-TA PAM silencing edit) |
| | 3248) | |
| 3249 | TTTAAATACTCACATTTTTAAGAAGAC (SEQ ID | RTT*1,6,11 (TG-to-TT PAM silencing edit) |
| | NO: 3249) | |
| 3250 | TTTAAATACTCACATTTTTAAGCAAAC (SEQ ID | RTT*2,7,11 |
| | NO: 3250) | |
| 3251 | TTTAAATACTCACATTTTTAAGGAGAC (SEQ ID | RTT*3,8,11 (TG-to-TC PAM silencing edit) |
| | NO: 3251) | |
| 3252 | TTTAAATACTCACATTTTTAAGTAAAC (SEQ ID | RTT*4,9,11 (TG-to-TA PAM silencing edit) |
| | NO: 3252) | |
| 3253 | TTTAAATACTCACATTTTTAAGTAGAC (SEQ ID | RTT*5,10,11 (TG-to-TA PAM silencing edit) |
| | NO: 3253) | |
| 3254 | TTTTAAATACTCACATTTTTAAGAAGAC (SEQ ID | RTT*1,6,11 (TG-to-TT PAM silencing edit) |
| | NO: 3254) | |
| 3255 | TTTTAAATACTCACATTTTTAAGCAAAC (SEQ ID | RTT*2,7,11 |
| | NO: 3255) | |
| 3256 | TTTTAAATACTCACATTTTTAAGGAGAC (SEQ ID | RTT*3,8,11 (TG-to-TC PAM silencing edit) |
| | NO: 3256) | |
| 3257 | TTTTAAATACTCACATTTTTAAGTAAAC (SEQ ID | RTT*4,9,11 (TG-to-TA PAM silencing edit) |
| | NO: 3257) | |
| 3258 | TTTTAAATACTCACATTTTTAAGTAGAC (SEQ ID | RTT*5,10,11 (TG-to-TA PAM silencing edit) |
| | NO: 3258) | |
| 3259 | ATTTTAAATACTCACATTTTTAAGAAGAC (SEQ ID | RTT*1,6,11 (TG-to-TT PAM silencing edit) |
| | NO: 3259) | |
| 3260 | ATTTTAAATACTCACATTTTTAAGCAAAC (SEQ ID | RTT*2,7,11 |
| | NO: 3260) | |
| 3261 | ATTTTAAATACTCACATTTTTAAGGAGAC (SEQ ID | RTT*3,8,11 (TG-to-TC PAM silencing edit) |
| | NO: 3261) | |
| 3262 | ATTTTAAATACTCACATTTTTAAGTAAAC (SEQ ID | RTT*4,9,11 (TG-to-TA PAM silencing edit) |
| | NO: 3262) | |
| 3263 | ATTTTAAATACTCACATTTTTAAGTAGAC (SEQ ID | RTT*5,10,11 (TG-to-TA PAM silencing edit) |
| | NO: 3263) | |
| 3264 | AATTTTAAATACTCACATTTTTAAGAAGAC (SEQ | RTT*1,6,11 (TG-to-TT PAM silencing edit) |
| | ID NO: 3264) | |
| 3265 | AATTTTAAATACTCACATTTTTAAGCAAAC (SEQ | RTT*2,7,11 |
| | ID NO: 3265) | |
| 3266 | AATTTTAAATACTCACATTTTTAAGGAGAC (SEQ | RTT*3,8,11 (TG-to-TC PAM silencing edit) |
| | ID NO: 3266) | |
| 3267 | AATTTTAAATACTCACATTTTTAAGTAAAC (SEQ | RTT*4,9,11 (TG-to-TA PAM silencing edit) |
| | ID NO: 3267) | |
| 3268 | AATTTTAAATACTCACATTTTTAAGTAGAC (SEQ | RTT*5,10,11 (TG-to-TA PAM silencing edit) |
| | ID NO: 3268) | |
| 3269 | AAATTTTAAATACTCACATTTTTAAGAAGAC | RTT*1,6,11 (TG-to-TT PAM silencing edit) |
| | (SEQ ID NO: 3269) | |
| 3270 | AAATTTTAAATACTCACATTTTTAAGCAAAC | RTT*2,7,11 |
| | (SEQ ID NO: 3270) | |
| 3271 | AAATTTTAAATACTCACATTTTTAAGGAGAC | RTT*3,8,11 (TG-to-TC PAM silencing edit) |
| | (SEQ ID NO: 3271) | |
| 3272 | AAATTTTAAATACTCACATTTTTAAGTAAAC | RTT*4,9,11 (TG-to-TA PAM silencing edit) |
| | (SEQ ID NO: 3272) | |
| 3273 | AAATTTTAAATACTCACATTTTTAAGTAGAC | RTT*5,10,11 (TG-to-TA PAM silencing edit) |
| | (SEQ ID NO: 3273) | |
| 3274 | TAAATTTTAAATACTCACATTTTTAAGAAGAC | RTT*1,6,11 (TG-to-TT PAM silencing edit) |
| | (SEQ ID NO: 3274) | |
| 3275 | TAAATTTTAAATACTCACATTTTTAAGCAAAC | RTT*2,7,11 |
| | (SEQ ID NO: 3275) | |
| 3276 | TAAATTTTAAATACTCACATTTTTAAGGAGAC | RTT*3,8,11 (TG-to-TC PAM silencing edit) |
| | (SEQ ID NO: 3276) | |
| 3277 | TAAATTTTAAATACTCACATTTTTAAGTAAAC | RTT*4,9,11 (TG-to-TA PAM silencing edit) |
| | (SEQ ID NO: 3277) | |
| 3278 | TAAATTTTAAATACTCACATTTTTAAGTAGAC | RTT*5,10,11 (TG-to-TA PAM silencing edit) |
| | (SEQ ID NO: 3278) | |
| 3279 | ATAAATTTTAAATACTCACATTTTTAAGAAGAC | RTT*1,6,11 (TG-to-TT PAM silencing edit) |
| | (SEQ ID NO: 3279) | |
| 3280 | ATAAATTTTAAATACTCACATTTTTAAGCAAAC | RTT*2,7,11 |
| | (SEQ ID NO: 3280) | |
| 3281 | ATAAATTTTAAATACTCACATTTTTAAGGAGAC | RTT*3,8,11 (TG-to-TC PAM silencing edit) |
| | (SEQ ID NO: 3281) | |
| 3282 | ATAAATTTTAAATACTCACATTTTTAAGTAAAC | RTT*4,9,11 (TG-to-TA PAM silencing edit) |
| | (SEQ ID NO: 3282) | |
| 3283 | ATAAATTTTAAATACTCACATTTTTAAGTAGAC | RTT*5,10,11 (TG-to-TA PAM silencing edit) |
| | (SEQ ID NO: 3283) | |
| 3284 | GATAAATTTTAAATACTCACATTTTTAAGAAGAC | RTT*1,6,11 (TG-to-TT PAM silencing edit) |
| | (SEQ ID NO: 3284) | |
| 3285 | GATAAATTTTAAATACTCACATTTTTAAGCAAAC | RTT*2,7,11 |
| | (SEQ ID NO: 3285) | |
| 3286 | GATAAATTTTAAATACTCACATTTTTAAGGAGAC | RTT*3,8,11 (TG-to-TC PAM silencing edit) |
| | (SEQ ID NO: 3286) | |
| 3287 | GATAAATTTTAAATACTCACATTTTTAAGTAAAC | RTT*4,9,11 (TG-to-TA PAM silencing edit) |
| | (SEQ ID NO: 3287) | |
| 3288 | GATAAATTTTAAATACTCACATTTTTAAGTAGAC | RTT*5,10,11 (TG-to-TA PAM silencing edit) |
| | (SEQ ID NO: 3288) | |
| 3289 | TGATAAATTTTAAATACTCACATTTTTAAGAAGA | RTT*1,6,11 (TG-to-TT PAM silencing edit) |
| | C (SEQ ID NO: 3289) | |
| 3290 | TGATAAATTTTAAATACTCACATTTTTAAGCAAA | RTT*2,7,11 |
| | C (SEQ ID NO: 3290) | |
| 3291 | TGATAAATTTTAAATACTCACATTTTTAAGGAGA | RTT*3,8,11 (TG-to-TC PAM silencing edit) |
| | C (SEQ ID NO: 3291) | |
| 3292 | TGATAAATTTTAAATACTCACATTTTTAAGTAAA | RTT*4,9,11 (TG-to-TA PAM silencing edit) |
| | C (SEQ ID NO: 3292) | |
| 3293 | TGATAAATTTTAAATACTCACATTTTTAAGTAGA | RTT*5,10,11 (TG-to-TA PAM silencing edit) |
| | C (SEQ ID NO: 3293) | |
| 3294 | GTGATAAATTTTAAATACTCACATTTTTAAGAAG | RTT*1,6,11 (TG-to-TT PAM silencing edit) |
| | AC (SEQ ID NO: 3294) | |
| 3295 | GTGATAAATTTTAAATACTCACATTTTTAAGCAA | RTT*2,7,11 |
| | AC (SEQ ID NO: 3295) | |
| 3296 | GTGATAAATTTTAAATACTCACATTTTTAAGGAG | RTT*3,8,11 (TG-to-TC PAM silencing edit) |
| | AC (SEQ ID NO: 3296) | |
| 3297 | GTGATAAATTTTAAATACTCACATTTTTAAGTAA | RTT*4,9,11 (TG-to-TA PAM silencing edit) |
| | AC (SEQ ID NO: 3297) | |
| 3298 | GTGATAAATTTTAAATACTCACATTTTTAAGTAG | RTT*5,10,11 (TG-to-TA PAM silencing edit) |
| | AC (SEQ ID NO: 3298) | |
| 3299 | AGTGATAAATTTTAAATACTCACATTTTTAAGAA | RTT*1,6,11 (TG-to-TT PAM silencing edit) |
| | GAC (SEQ ID NO: 3299) | |
| 3300 | AGTGATAAATTTTAAATACTCACATTTTTAAGCA | RTT*2,7,11 |
| | AAC (SEQ ID NO: 3300) | |
| 3301 | AGTGATAAATTTTAAATACTCACATTTTTAAGGA | RTT*3,8,11 (TG-to-TC PAM silencing edit) |
| | GAC (SEQ ID NO: 3301) | |
| 3302 | AGTGATAAATTTTAAATACTCACATTTTTAAGTA | RTT*4,9,11 (TG-to-TA PAM silencing edit) |
| | AAC (SEQ ID NO: 3302) | |
| 3303 | AGTGATAAATTTTAAATACTCACATTTTTAAGTA | RTT*5,10,11 (TG-to-TA PAM silencing edit) |
| | GAC (SEQ ID NO: 3303) | |
| 3304 | AAGTGATAAATTTTAAATACTCACATTTTTAAGA | RTT*1,6,11 (TG-to-TT PAM silencing edit) |
| | AGAC (SEQ ID NO: 3304) | |
| 3305 | AAGTGATAAATTTTAAATACTCACATTTTTAAGC | RTT*2,7,11 |
| | AAAC (SEQ ID NO: 3305) | |
| 3306 | AAGTGATAAATTTTAAATACTCACATTTTTAAGG | RTT*3,8,11 (TG-to-TC PAM silencing edit) |
| | AGAC (SEQ ID NO: 3306) | |
| 3307 | AAGTGATAAATTTTAAATACTCACATTTTTAAGT | RTT*4,9,11 (TG-to-TA PAM silencing edit) |
| | AAAC (SEQ ID NO: 3307) | |
| 3308 | AAGTGATAAATTTTAAATACTCACATTTTTAAGT | RTT*5,10,11 (TG-to-TA PAM silencing edit) |
| | AGAC (SEQ ID NO: 3308) | |
| 3309 | AAAGTGATAAATTTTAAATACTCACATTTTTAAG | RTT*1,6,11 (TG-to-TT PAM silencing edit) |
| | AAGAC (SEQ ID NO: 3309) | |
| 3310 | AAAGTGATAAATTTTAAATACTCACATTTTTAAG | RTT*2,7,11 |
| | CAAAC (SEQ ID NO: 3310) | |
| 3311 | AAAGTGATAAATTTTAAATACTCACATTTTTAAG | RTT*3,8,11 (TG-to-TC PAM silencing edit) |
| | GAGAC (SEQ ID NO: 3311) | |
| 3312 | AAAGTGATAAATTTTAAATACTCACATTTTTAAG | RTT*4,9,11 (TG-to-TA PAM silencing edit) |
| | TAAAC (SEQ ID NO: 3312) | |
| 3313 | AAAGTGATAAATTTTAAATACTCACATTTTTAAG | RTT*5,10,11 (TG-to-TA PAM silencing edit) |
| | TAGAC (SEQ ID NO: 3313) | |
| 3314 | AAAAGTGATAAATTTTAAATACTCACATTTTTAA | RTT*1,6,11 (TG-to-TT PAM silencing edit) |
| | GAAGAC (SEQ ID NO: 3314) | |
| 3315 | AAAAGTGATAAATTTTAAATACTCACATTTTTAA | RTT*2,7,11 |
| | GCAAAC (SEQ ID NO: 3315) | |
| 3316 | AAAAGTGATAAATTTTAAATACTCACATTTTTAA | RTT*3,8,11 (TG-to-TC PAM silencing edit) |
| | GGAGAC (SEQ ID NO: 3316) | |
| 3317 | AAAAGTGATAAATTTTAAATACTCACATTTTTAA | RTT*4,9,11 (TG-to-TA PAM silencing edit) |
| | GTAAAC (SEQ ID NO: 3317) | |
| 3318 | AAAAGTGATAAATTTTAAATACTCACATTTTTAA | RTT*5,10,11 (TG-to-TA PAM silencing edit) |
| | GTAGAC (SEQ ID NO: 3318) | |
| 2885 | AAAAGTTAAACATCTCTTCT (SEQ ID NO: 2885) | PE3 ngRNA spacer (GG PAM) |
| 2559 | AAACATCTCTTCTGGAGGAC (SEQ ID NO: 2559) | PE3 ngRNA spacer (TG PAM) |
| 2886 | AAGTTAAACATCTCTTCTGG (SEQ ID NO: 2886) | PE3 ngRNA spacer (AG PAM) |
| 2573 | AGCATTAAACATTTCAAAAG (SEQ ID NO: 2573) | PE3 ngRNA spacer (TG PAM) |
| 2887 | AGTTAAACATCTCTTCTGGA (SEQ ID NO: 2887) | PE3 ngRNA spacer (GG PAM) |
| 2888 | ATATTTCCATTTACTCTTTT (SEQ ID NO: 2888) | PE3 ngRNA spacer (TG PAM) |
| 2889 | CATTTACTCTTTTTGCTGAT (SEQ ID NO: 2889) | PE3 ngRNA spacer (GG PAM) |
| 2890 | CCATTTACTCTTTTTGCTGA (SEQ ID NO: 2890) | PE3 ngRNA spacer (TG PAM) |
| 3319 | CTCACATTTTTAAGAAGACC (SEQ ID NO: 3319) | PE3b*1 ngRNA spacer (AG PAM) |
| 2891 | CTCACATTTTTAAGCAAACC (SEQ ID NO: 2891) | PE3b*2 ngRNA spacer (AG PAM) |
| 3320 | CTCACATTTTTAAGGAGACC (SEQ ID NO: 3320) | PE3b*3 ngRNA spacer (AG PAM) |
| 3321 | CTCACATTTTTAAGTAAACC (SEQ ID NO: 3321) | PE3b*4 ngRNA spacer (AG PAM) |
| 3322 | CTCACATTTTTAAGTAGACC (SEQ ID NO: 3322) | PE3b*5 ngRNA spacer (AG PAM) |
| 2892 | GAAAAGTTAAACATCTCTTC (SEQ ID NO: 2892) | PE3 ngRNA spacer (TG PAM) |
| 3323 | TCACATTTTTAAGAAGACCA (SEQ ID NO: 3323) | PE3b*6 ngRNA spacer (GG PAM) |
| 2893 | TCACATTTTTAAGCAAACCA (SEQ ID NO: 2893) | PE3b*7 ngRNA spacer (GG PAM) |
| 3324 | TCACATTTTTAAGGAGACCA (SEQ ID NO: 3324) | PE3b*8 ngRNA spacer (GG PAM) |
| 3325 | TCACATTTTTAAGTAAACCA (SEQ ID NO: 3325) | PE3b*9 ngRNA spacer (GG PAM) |
| 3326 | TCACATTTTTAAGTAGACCA (SEQ ID NO: 3326) | PE3b*10 ngRNA spacer (GG PAM) |
| 2569 | TCAGCATTAAACATTTCAAA (SEQ ID NO: 2569) | PE3 ngRNA spacer (AG PAM) |
| 2894 | TTTAAATACTCACATTTTTA (SEQ ID NO: 2894) | PE3b*11 ngRNA spacer (AG PAM) |
| 2895 | TTTCCATTTACTCTTTTTGC (SEQ ID NO: 2895) | PE3 ngRNA spacer (TG PAM) |
| 2896 | TTTTTGCTGATGGCACATTC (SEQ ID NO: 2896) | PE3 ngRNA spacer (AG PAM) |
| 2897 | AAGTTAAACATCTCTTCTGGGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2898 | GAAAAGTTAAACATCTCTTCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 2898) | |
| 2899 | AAGTTAAACATCTCTTCTGGGTTTTAGAGCTAGA | PE3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |
| 2900 | GAAAAGTTAAACATCTCTTCGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2900) | |

TABLE 48

| Sequence | | |
|---|---|---|
| Number | Sequence | Description |
| 3327 | CCTGGTTTGCTTAAAAA (SEQ ID NO: 3327) | PEgRNA spacer (TG PAM) |
| 3328 | TCCTGGTTTGCTTAAAAA (SEQ ID NO: 3328) | PEgRNA spacer (TG PAM) |
| 3329 | ATCCTGGTTTGCTTAAAAA (SEQ ID NO: 3329) | PEgRNA spacer (TG PAM) |
| 2625 | TATCCTGGTTTGCTTAAAAA (SEQ ID NO: 2625) | PEgRNA spacer (TG PAM) |
| 3330 | CTATCCTGGTTTGCTTAAAAA (SEQ ID NO: 3330) | PEgRNA spacer (TG PAM) |
| 3331 | ACTATCCTGGTTTGCTTAAAAA (SEQ ID NO: 3331) | PEgRNA spacer (TG PAM) |
| 3332 | TTAAG | PBS |
| 3333 | TTAAGC | PBS |
| 3334 | TTAAGCA | PBS |
| 3335 | TTAAGCAA | PBS |
| 3336 | TTAAGCAAA | PBS |
| 3337 | TTAAGCAAAC (SEQ ID NO: 3337) | PBS |
| 3338 | TTAAGCAAACC (SEQ ID NO: 3338) | PBS |
| 3339 | TTAAGCAAACCA (SEQ ID NO: 3339) | PBS |
| 3340 | TTAAGCAAACCAG (SEQ ID NO: 3340) | PBS |
| 3341 | TTAAGCAAACCAGG (SEQ ID NO: 3341) | PBS |
| 3342 | TTAAGCAAACCAGGA (SEQ ID NO: 3342) | PBS |
| 3343 | TTAAGCAAACCAGGAT (SEQ ID NO: 3343) | PBS |
| 3344 | TTAAGCAAACCAGGATA (SEQ ID NO: 3344) | PBS |
| 3345 | TTAAGCAAACCAGGATAG (SEQ ID NO: 3345) | PBS |
| 3346 | TTAAGCAAACCAGGATAGT (SEQ ID NO: 3346) | PBS |
| 3347 | ACTCACATTT (SEQ ID NO: 3347) | RTT |
| 3348 | TACTCACATTT (SEQ ID NO: 3348) | RTT |
| 3349 | ATACTCACATTT (SEQ ID NO: 3349) | RTT |
| 3350 | AATACTCACATTT (SEQ ID NO: 3350) | RTT |
| 3351 | AAATACTCACATTT (SEQ ID NO: 3351) | RTT |
| 3352 | TAAATACTCACATTT (SEQ ID NO: 3352) | RTT |
| 3353 | TTAAATACTCACATTT (SEQ ID NO: 3353) | RTT |
| 3354 | TTTAAATACTCACATTT (SEQ ID NO: 3354) | RTT |
| 3355 | TTTTAAATACTCACATTT (SEQ ID NO: 3355) | RTT |
| 3356 | ATTTTAAATACTCACATTT (SEQ ID NO: 3356) | RTT |
| 3357 | AATTTTAAATACTCACATTT (SEQ ID NO: 3357) | RTT |
| 3358 | AAATTTTAAATACTCACATTT (SEQ ID NO: 3358) | RTT |
| 3359 | TAAATTTTAAATACTCACATTT (SEQ ID NO: 3359) | RTT |
| 3360 | ATAAATTTTAAATACTCACATTT (SEQ ID NO: | RTT |
| | 3360) | |
| 3361 | GATAAATTTTAAATACTCACATTT (SEQ ID NO: | RTT |
| | 3361) | |
| 3362 | TGATAAATTTTAAATACTCACATTT (SEQ ID NO: | RTT |
| | 3362) | |
| 3363 | GTGATAAATTTTAAATACTCACATTT (SEQ ID NO: | RTT |
| | 3363) | |
| 3364 | AGTGATAAATTTTAAATACTCACATTT (SEQ ID | RTT |
| | NO: 3364) | |
| 3365 | AAGTGATAAATTTTAAATACTCACATTT (SEQ ID | RTT |
| | NO: 3365) | |
| 3366 | AAAGTGATAAATTTTAAATACTCACATTT (SEQ | RTT |
| | ID NO: 3366) | |
| 3367 | AAAAGTGATAAATTTTAAATACTCACATTT (SEQ | RTT |
| | ID NO: 3367) | |
| 3368 | CAAAAGTGATAAATTTTAAATACTCACATTT | RTT |
| | (SEQ ID NO: 3368) | |
| 3369 | TCAAAAGTGATAAATTTTAAATACTCACATTT | RTT |
| | (SEQ ID NO: 3369) | |
| 3370 | TTCAAAAGTGATAAATTTTAAATACTCACATTT | RTT |
| | (SEQ ID NO: 3370) | |
| 3371 | TTTCAAAAGTGATAAATTTTAAATACTCACATTT | RTT |
| | (SEQ ID NO: 3371) | |
| 3372 | ATTTCAAAAGTGATAAATTTTAAATACTCACATT | RTT |
| | T (SEQ ID NO: 3372) | |
| 3373 | CATTTCAAAAGTGATAAATTTTAAATACTCACAT | RTT |
| | TT (SEQ ID NO: 3373) | |
| 3374 | ACATTTCAAAAGTGATAAATTTTAAATACTCACA | RTT |
| | TTT (SEQ ID NO: 3374) | |
| 3375 | AACATTTCAAAAGTGATAAATTTTAAATACTCAC | RTT |
| | ATTT (SEQ ID NO: 3375) | |
| 3376 | AAACATTTCAAAAGTGATAAATTTTAAATACTCA | RTT |
| | CATTT (SEQ ID NO: 3376) | |
| 3377 | TAAACATTTCAAAAGTGATAAATTTTAAATACTC | RTT |
| | ACATTT (SEQ ID NO: 3377) | |
| 2885 | AAAAGTTAAACATCTCTTCT (SEQ ID NO: 2885) | PE3 ngRNA spacer (GG PAM) |
| 2559 | AAACATCTCTTCTGGAGGAC (SEQ ID NO: 2559) | PE3 ngRNA spacer (TG PAM) |
| 2886 | AAGTTAAACATCTCTTCTGG (SEQ ID NO: 2886) | PE3 ngRNA spacer (AG PAM) |
| 3084 | AGAAGAGATAAACAAAGAAA (SEQ ID NO: 3084) | PE3 ngRNA spacer (AG PAM) |
| 2573 | AGCATTAAACATTTCAAAAG (SEQ ID NO: 2573) | PE3 ngRNA spacer (TG PAM) |
| 3085 | AGGTAAGAAGAGATAAACAA (SEQ ID NO: 3085) | PE3 ngRNA spacer (AG PAM) |
| 2887 | AGTTAAACATCTCTTCTGGA (SEQ ID NO: 2887) | PE3 ngRNA spacer (GG PAM) |
| 2888 | ATATTTCCATTTACTCTTTT (SEQ ID NO: 2888) | PE3 ngRNA spacer (TG PAM) |
| 2889 | CATTTACTCTTTTTGCTGAT (SEQ ID NO: 2889) | PE3 ngRNA spacer (GG PAM) |
| 2890 | CCATTTACTCTTTTTGCTGA (SEQ ID NO: 2890) | PE3 ngRNA spacer (TG PAM) |
| 2892 | GAAAAGTTAAACATCTCTTC (SEQ ID NO: 2892) | PE3 ngRNA spacer (TG PAM) |
| 2569 | TCAGCATTAAACATTTCAAA (SEQ ID NO: 2569) | PE3 ngRNA spacer (AG PAM) |
| 2894 | TTTAAATACTCACATTTTTA (SEQ ID NO: 2894) | PE3b ngRNA spacer (AG PAM) |
| 2895 | TTTCCATTTACTCTTTTTGC (SEQ ID NO: 2895) | PE3 ngRNA spacer (TG PAM) |
| 2896 | TTTTTGCTGATGGCACATTC (SEQ ID NO: 2896) | PE3 ngRNA spacer (AG PAM) |
| 2897 | AAGTTAAACATCTCTTCTGGGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2898 | GAAAAGTTAAACATCTCTTCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 2898) | |
| 2899 | AAGTTAAACATCTCTTCTGGGTTTTAGAGCTAGA | PE3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |
| 2900 | GAAAAGTTAAACATCTCTTCGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2900) | |

TABLE 49

| Sequence | | |
|---|---|---|
| Number | Sequence | Description |
| 3378 | GCATTAAACATTTCAAA (SEQ ID NO: 3378) | PEgRNA spacer (AG;AGTGAT PAM) |
| 3379 | AGCATTAAACATTTCAAA (SEQ ID NO: 3379) | PEgRNA spacer (AG;AGTGAT PAM) |
| 3380 | CAGCATTAAACATTTCAAA (SEQ ID NO: 3380) | PEgRNA spacer (AG;AGTGAT PAM) |
| 2569 | TCAGCATTAAACATTTCAAA (SEQ ID NO: 2569) | PEgRNA spacer (AG;AGTGAT PAM) |
| 3381 | TTCAGCATTAAACATTTCAAA (SEQ ID NO: 3381) | PEgRNA spacer (AG;AGTGAT PAM) |
| 3382 | ATTCAGCATTAAACATTTCAAA (SEQ ID NO: 3382) | PEgRNA spacer (AG;AGTGAT PAM) |
| 3383 | GAAAT | PBS |
| 3384 | GAAATG | PBS |
| 3385 | GAAATGT | PBS |
| 3386 | GAAATGTT | PBS |
| 3387 | GAAATGTTT | PBS |
| 3388 | GAAATGTTTA (SEQ ID NO: 3388) | PBS |
| 3389 | GAAATGTTTAA (SEQ ID NO: 3389) | PBS |
| 3390 | GAAATGTTTAAT (SEQ ID NO: 3390) | PBS |
| 3391 | GAAATGTTTAATG (SEQ ID NO: 3391) | PBS |
| 3392 | GAAATGTTTAATGC (SEQ ID NO: 3392) | PBS |
| 3393 | GAAATGTTTAATGCT (SEQ ID NO: 3393) | PBS |
| 3394 | GAAATGTTTAATGCTG (SEQ ID NO: 3394) | PBS |
| 3395 | GAAATGTTTAATGCTGA (SEQ ID NO: 3395) | PBS |
| 3396 | GAAATGTTTAATGCTGAA (SEQ ID NO: 3396) | PBS |
| 3397 | GAAATGTTTAATGCTGAAT (SEQ ID NO: 3397) | PBS |
| 3398 | AGTATTTAAAATTTATCACTTTT (SEQ ID NO: | RTT |
| | 3398) | |
| 3399 | GAGTATTTAAAATTTATCACTTTT (SEQ ID NO: | RTT |
| | 3399) | |
| 3400 | TGAGTATTTAAAATTTATCACTTTT (SEQ ID NO: | RTT |
| | 3400) | |
| 3401 | GTGAGTATTTAAAATTTATCACTTTT (SEQ ID NO: | RTT |
| | 3401) | |
| 3402 | TGTGAGTATTTAAAATTTATCACTTTT (SEQ ID | RTT |
| | NO: 3402) | |
| 3403 | ATGTGAGTATTTAAAATTTATCACTTTT (SEQ ID | RTT |
| | NO: 3403) | |
| 3404 | AATGTGAGTATTTAAAATTTATCACTTTT (SEQ ID | RTT |
| | NO: 3404) | |
| 3405 | AAATGTGAGTATTTAAAATTTATCACTTTT (SEQ | RTT |
| | ID NO: 3405) | |
| 3406 | AAAATGTGAGTATTTAAAATTTATCACTTTT (SEQ | RTT |
| | ID NO: 3406) | |
| 3407 | AAAAATGTGAGTATTTAAAATTTATCACTTTT | RTT |
| | (SEQ ID NO: 3407) | |
| 3408 | TAAAAATGTGAGTATTTAAAATTTATCACTTTT | RTT |
| | (SEQ ID NO: 3408) | |
| 3409 | TTAAAAATGTGAGTATTTAAAATTTATCACTTTT | RTT |
| | (SEQ ID NO: 3409) | |
| 3410 | CTTAAAAATGTGAGTATTTAAAATTTATCACTTT | RTT |
| | T (SEQ ID NO: 3410) | |
| 3411 | GCTTAAAAATGTGAGTATTTAAAATTTATCACTT | RTT |
| | TT (SEQ ID NO: 3411) | |
| 3412 | TGCTTAAAAATGTGAGTATTTAAAATTTATCACT | RTT |
| | TTT (SEQ ID NO: 3412) | |
| 3413 | TTGCTTAAAAATGTGAGTATTTAAAATTTATCAC | RTT |
| | TTTT (SEQ ID NO: 3413) | |
| 3414 | TTTGCTTAAAAATGTGAGTATTTAAAATTTATCA | RTT |
| | CTTTT (SEQ ID NO: 3414) | |
| 3415 | GTTTGCTTAAAAATGTGAGTATTTAAAATTTATC | RTT |
| | ACTTTT (SEQ ID NO: 3415) | |
| 2943 | AAGAAGTTGCTCTTTTCACT (SEQ ID NO: 2943) | PE3 ngRNA spacer (CAAGGT PAM) |
| 2533 | AAGGTCTTGGGTATGCACCT (SEQ ID NO: 2533) | PE3 ngRNA spacer (ATAGAT PAM) |
| 2611 | AAGTTGCTCTTTTCACTCAA (SEQ ID NO: 2611) | PE3 ngRNA spacer (GG PAM) |
| 2612 | ATACTTTCAGCACTCAGATT (SEQ ID NO: 2612) | PE3 ngRNA spacer (TG PAM) |
| 2944 | ATATACTTTCAGCACTCAGA (SEQ ID NO: 2944) | PE3 ngRNA spacer (TTTGAT PAM) |
| 2945 | ATCTCATATACTTTCAGCAC (SEQ ID NO: 2945) | PE3 ngRNA spacer (TCAGAT PAM) |
| 2677 | ATGCACCTATAGATTACTAT (SEQ ID NO: 2677) | PE3 ngRNA spacer (CCTGGT PAM) |
| 2771 | ATTACTATCCTGGTTTGCTT (SEQ ID NO: 2771) | PE3 ngRNA spacer (AAAAAT PAM) |
| 2613 | CACCTATAGATTACTATCCT (SEQ ID NO: 2613) | PE3 ngRNA spacer (GG PAM) |
| 2614 | CTCAGATTTGATAAAGAAGT (SEQ ID NO: 2614) | PE3 ngRNA spacer (TG PAM) |
| 2615 | CTCATATACTTTCAGCACTC (SEQ ID NO: 2615) | PE3 ngRNA spacer (AG PAM) |
| 2616 | CTCTTTTCACTCAAGGTCTT (SEQ ID NO: 2616) | PE3 ngRNA spacer (GG PAM) |
| 2617 | CTGGTTTGCTTAAAAATGTG (SEQ ID NO: 2617) | PE3 ngRNA spacer (TG PAM) |
| 2618 | GAAGTTGCTCTTTTCACTCA (SEQ ID NO: 2618) | PE3 ngRNA spacer (AG PAM) |
| 2619 | GCACCTATAGATTACTATCC (SEQ ID NO: 2619) | PE3 ngRNA spacer (TG PAM) |
| 2620 | GCACTCAGATTTGATAAAGA (SEQ ID NO: 2620) | PE3 ngRNA spacer (AG PAM) |
| 2621 | GCTCTTTTCACTCAAGGTCT (SEQ ID NO: 2621) | PE3 ngRNA spacer (TG PAM) |
| 2622 | GGTCTTGGGTATGCACCTAT (SEQ ID NO: 2622) | PE3 ngRNA spacer (AG PAM) |
| 2623 | GTATTATCTCATATACTTTC (SEQ ID NO: 2623) | PE3 ngRNA spacer (AG PAM) |
| 2624 | TATAGATTACTATCCTGGTT (SEQ ID NO: 2624) | PE3 ngRNA spacer (TG PAM) |
| 2625 | TATCCTGGTTTGCTTAAAAA (SEQ ID NO: 2625) | PE3 ngRNA spacer (TG PAM) |
| 2626 | TATTTAAAATTTATCACTTT (SEQ ID NO: 2626) | PE3 ngRNA spacer (TG;TGAAAT PAM) |
| 2627 | TCAGCACTCAGATTTGATAA (SEQ ID NO: 2627) | PE3 ngRNA spacer (AG;AGAAGT PAM) |
| 2628 | TCCTGGTTTGCTTAAAAATG (SEQ ID NO: 2628) | PE3 ngRNA spacer (TG PAM) |
| 2629 | TCTTTTCACTCAAGGTCTTG (SEQ ID NO: 2629) | PE3 ngRNA spacer (GG PAM) |
| 2946 | TGCTCTTTTCACTCAAGGTC (SEQ ID NO: 2946) | PE3 ngRNA spacer (TTGGGT PAM) |
| 2947 | TGCTTAAAAATGTGAGTATT (SEQ ID NO: 2947) | PE3b ngRNA spacer (TAAAAT PAM) |
| 2630 | TTCACTCAAGGTCTTGGGTA (SEQ ID NO: 2630) | PE3 ngRNA spacer (TG PAM) |

TABLE 50

| Sequence | | |
|---|---|---|
| Number | Sequence | Description |
| 3416 | TGGTTTGCTTAAAAATG (SEQ ID NO: 3416) | PEgRNA spacer (TG PAM) |
| 3417 | CTGGTTTGCTTAAAAATG (SEQ ID NO: 3417) | PEgRNA spacer (TG PAM) |
| 3418 | CCTGGTTTGCTTAAAAATG (SEQ ID NO: 3418) | PEgRNA spacer (TG PAM) |
| 2628 | TCCTGGTTTGCTTAAAAATG (SEQ ID NO: 2628) | PEgRNA spacer (TG PAM) |
| 3419 | ATCCTGGTTTGCTTAAAAATG (SEQ ID NO: 3419) | PEgRNA spacer (TG PAM) |
| 3420 | TATCCTGGTTTGCTTAAAAATG (SEQ ID NO: 3420) | PEgRNA spacer (TG PAM) |
| 3421 | TTTTA | PBS |
| 3422 | TTTTAA | PBS |
| 3423 | TTTTAAG | PBS |
| 3424 | TTTTAAGC | PBS |
| 3425 | TTTTAAGCA | PBS |
| 3426 | TTTTAAGCAA (SEQ ID NO: 3426) | PBS |
| 3427 | TTTTAAGCAAA (SEQ ID NO: 3427) | PBS |
| 3428 | TTTTAAGCAAAC (SEQ ID NO: 3428) | PBS |
| 3429 | TTTTAAGCAAACC (SEQ ID NO: 3429) | PBS |
| 3430 | TTTTAAGCAAACCA (SEQ ID NO: 3430) | PBS |
| 3431 | TTTTAAGCAAACCAG (SEQ ID NO: 3431) | PBS |
| 3432 | TTTTAAGCAAACCAGG (SEQ ID NO: 3432) | PBS |
| 3433 | TTTTAAGCAAACCAGGA (SEQ ID NO: 3433) | PBS |
| 3434 | TTTTAAGCAAACCAGGAT (SEQ ID NO: 3434) | PBS |
| 3435 | TTTTAAGCAAACCAGGATA (SEQ ID NO: 3435) | PBS |
| 3436 | ATACTCACAT (SEQ ID NO: 3436) | RTT |
| 3437 | AATACTCACAT (SEQ ID NO: 3437) | RTT |
| 3438 | AAATACTCACAT (SEQ ID NO: 3438) | RTT |
| 3439 | TAAATACTCACAT (SEQ ID NO: 3439) | RTT |
| 3440 | TTAAATACTCACAT (SEQ ID NO: 3440) | RTT |
| 3441 | TTTAAATACTCACAT (SEQ ID NO: 3441) | RTT |
| 3442 | TTTTAAATACTCACAT (SEQ ID NO: 3442) | RTT |
| 3443 | ATTTTAAATACTCACAT (SEQ ID NO: 3443) | RTT |
| 3444 | AATTTTAAATACTCACAT (SEQ ID NO: 3444) | RTT |
| 3445 | AAATTTTAAATACTCACAT (SEQ ID NO: 3445) | RTT |
| 3446 | TAAATTTTAAATACTCACAT (SEQ ID NO: 3446) | RTT |
| 3447 | ATAAATTTTAAATACTCACAT (SEQ ID NO: 3447) | RTT |
| 3448 | GATAAATTTTAAATACTCACAT (SEQ ID NO: 3448) | RTT |
| 3449 | TGATAAATTTTAAATACTCACAT (SEQ ID NO: | RTT |
| | 3449) | |
| 3450 | GTGATAAATTTTAAATACTCACAT (SEQ ID NO: | RTT |
| | 3450) | |
| 3451 | AGTGATAAATTTTAAATACTCACAT (SEQ ID NO: | RTT |
| | 3451) | |
| 3452 | AAGTGATAAATTTTAAATACTCACAT (SEQ ID | RTT |
| | NO: 3452) | |
| 3453 | AAAGTGATAAATTTTAAATACTCACAT (SEQ ID | RTT |
| | NO: 3453) | |
| 3454 | AAAAGTGATAAATTTTAAATACTCACAT (SEQ ID | RTT |
| | NO: 3454) | |
| 3455 | CAAAAGTGATAAATTTTAAATACTCACAT (SEQ | RTT |
| | ID NO: 3455 | |
| 3456 | TCAAAAGTGATAAATTTTAAATACTCACAT (SEQ | RTT |
| | ID NO: 3456) | |
| 3457 | TTCAAAAGTGATAAATTTTAAATACTCACAT | RTT |
| | (SEQ ID NO: 3457) | |
| 3458 | TTTCAAAAGTGATAAATTTTAAATACTCACAT | RTT |
| | (SEQ ID NO: 3458) | |
| 3459 | ATTTCAAAAGTGATAAATTTTAAATACTCACAT | RTT |
| | (SEQ ID NO: 3459) | |
| 3460 | CATTTCAAAAGTGATAAATTTTAAATACTCACAT | RTT |
| | (SEQ ID NO: 3460) | |
| 3461 | ACATTTCAAAAGTGATAAATTTTAAATACTCACA | RTT |
| | T (SEQ ID NO: 3461) | |
| 3462 | AACATTTCAAAAGTGATAAATTTTAAATACTCAC | RTT |
| | AT (SEQ ID NO: 3462) | |
| 3463 | AAACATTTCAAAAGTGATAAATTTTAAATACTCA | RTT |
| | CAT (SEQ ID NO: 3463) | |
| 3464 | TAAACATTTCAAAAGTGATAAATTTTAAATACTC | RTT |
| | ACAT (SEQ ID NO: 3464) | |
| 3465 | TTAAACATTTCAAAAGTGATAAATTTTAAATACT | RTT |
| | CACAT (SEQ ID NO: 3465) | |
| 3466 | ATTAAACATTTCAAAAGTGATAAATTTTAAATAC | RTT |
| | TCACAT (SEQ ID NO: 3466) | |
| 2885 | AAAAGTTAAACATCTCTTCT (SEQ ID NO: 2885) | PE3 ngRNA spacer (GG PAM) |
| 2559 | AAACATCTCTTCTGGAGGAC (SEQ ID NO: 2559) | PE3 ngRNA spacer (TG PAM) |
| 2886 | AAGTTAAACATCTCTTCTGG (SEQ ID NO: 2886) | PE3 ngRNA spacer (AG PAM) |
| 3084 | AGAAGAGATAAACAAAGAAA (SEQ ID NO: 3084) | PE3 ngRNA spacer (AG PAM) |
| 2573 | AGCATTAAACATTTCAAAAG (SEQ ID NO: 2573) | PE3 ngRNA spacer (TG PAM) |
| 3085 | AGGTAAGAAGAGATAAACAA (SEQ ID NO: 3085) | PE3 ngRNA spacer (AG PAM) |
| 2887 | AGTTAAACATCTCTTCTGGA (SEQ ID NO: 2887) | PE3 ngRNA spacer (GG PAM) |
| 2888 | ATATTTCCATTTACTCTTTT (SEQ ID NO: 2888) | PE3 ngRNA spacer (TG PAM) |
| 2889 | CATTTACTCTTTTTGCTGAT (SEQ ID NO: 2889) | PE3 ngRNA spacer (GG PAM) |
| 2890 | CCATTTACTCTTTTTGCTGA (SEQ ID NO: 2890) | PE3 ngRNA spacer (TG PAM) |
| 2892 | GAAAAGTTAAACATCTCTTC (SEQ ID NO: 2892) | PE3 ngRNA spacer (TG PAM) |
| 2569 | TCAGCATTAAACATTTCAAA (SEQ ID NO: 2569) | PE3 ngRNA spacer (AG PAM) |
| 2895 | TTTCCATTTACTCTTTTTGC (SEQ ID NO: 2895) | PE3 ngRNA spacer (TG PAM) |
| 2896 | TTTTTGCTGATGGCACATTC (SEQ ID NO: 2896) | PE3 ngRNA spacer (AG PAM) |
| 2897 | AAGTTAAACATCTCTTCTGGGTTTTAGAGCTAGA | PE3 ngRNA (AG PAM) |
| | AATAGCAAGTTAAAATAAGGCTAGTCCGTTATCA | |
| | ACTTGAAAAAGTGGCACCGAGTCGGTGC (SEQ ID | |
| | NO: 2897) | |
| 2898 | GAAAAGTTAAACATCTCTTCGTTTTAGAGCTAGA | PE3 ngRNA (TG PAM) |
| | AATAGCAAGTTAAAATAAGGCTAGTCCGTTATCA | |
| | ACTTGAAAAAGTGGCACCGAGTCGGTGC (SEQ ID | |
| | NO: 2898) | |
| 2899 | AAGTTAAACATCTCTTCTGGGTTTTAGAGCTAGA | PE3 ngRNA (AG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2899) | |
| 2900 | GAAAAGTTAAACATCTCTTCGTTTTAGAGCTAGA | PE3 ngRNA (TG PAM), contains end |
| | AATAGCAAGTTAAAATAAGGCTAGTCCGTTATCA | adaptations |
| | ACTTGAAAAAGTGGCACCGAGTCGGTGCTTTT | |
| | (SEQ ID NO: 2900) | |

TABLE 51

| Sequence | | |
|---|---|---|
| Number | Sequence | Description |
| 3467 | ACCTATAGATTACTATC (SEQ ID NO: 3467) | PEgRNA spacer (CTGG PAM) |
| 3468 | CACCTATAGATTACTATC (SEQ ID NO: 3468) | PEgRNA spacer (CTGG PAM) |
| 3469 | GCACCTATAGATTACTATC (SEQ ID NO: 3469) | PEgRNA spacer (CTGG PAM) |
| 3470 | TGCACCTATAGATTACTATC (SEQ ID NO: 3470) | PEgRNA spacer (CTGG PAM) |
| 3471 | ATGCACCTATAGATTACTATC (SEQ ID NO: 3471) | PEgRNA spacer (CTGG PAM) |
| 3472 | TATGCACCTATAGATTACTATC (SEQ ID NO: 3472) | PEgRNA spacer (CTGG PAM) |
| 3473 | AGTAA | PBS |
| 3474 | AGTAAT | PBS |
| 3475 | AGTAATC | PBS |
| 3476 | AGTAATCT | PBS |
| 3477 | AGTAATCTA | PBS |
| 3478 | AGTAATCTAT (SEQ ID NO: 3478) | PBS |
| 3479 | AGTAATCTATA (SEQ ID NO: 3479) | PBS |
| 3480 | AGTAATCTATAG (SEQ ID NO: 3480) | PBS |
| 3481 | AGTAATCTATAGG (SEQ ID NO: 3481) | PBS |
| 3482 | AGTAATCTATAGGT (SEQ ID NO: 3482) | PBS |
| 3483 | AGTAATCTATAGGTG (SEQ ID NO: 3483) | PBS |
| 3484 | AGTAATCTATAGGTGC (SEQ ID NO: 3484) | PBS |
| 3485 | AGTAATCTATAGGTGCA (SEQ ID NO: 3485) | PBS |
| 3486 | AGTAATCTATAGGTGCAT (SEQ ID NO: 3486) | PBS |
| 3487 | AGTAATCTATAGGTGCATA (SEQ ID NO: 3487) | PBS |
| 3488 | TCACATTTTTAAGCAAACCAGGAT (SEQ ID NO: | RTT |
| | 3488) | |
| 3489 | CTCACATTTTTAAGCAAACCAGGAT (SEQ ID NO: | RTT |
| | 3489) | |
| 3490 | ACTCACATTTTTAAGCAAACCAGGAT (SEQ ID | RTT |
| | NO: 3490) | |
| 3491 | TACTCACATTTTTAAGCAAACCAGGAT (SEQ ID | RTT |
| | NO: 3491) | |
| 3492 | ATACTCACATTTTTAAGCAAACCAGGAT (SEQ ID | RTT |
| | NO: 3492) | |
| 3493 | AATACTCACATTTTTAAGCAAACCAGGAT (SEQ | RTT |
| | ID NO: 3493) | |
| 3494 | AAATACTCACATTTTTAAGCAAACCAGGAT (SEQ | RTT |
| | ID NO: 3494) | |
| 3495 | TAAATACTCACATTTTTAAGCAAACCAGGAT | RTT |
| | (SEQ ID NO: 3495) | |
| 3496 | TTAAATACTCACATTTTTAAGCAAACCAGGAT | RTT |
| | (SEQ ID NO: 3496) | |
| 3497 | TTTAAATACTCACATTTTTAAGCAAACCAGGAT | RTT |
| | (SEQ ID NO: 3497) | |
| 3498 | TTTTAAATACTCACATTTTTAAGCAAACCAGGAT | RTT |
| | (SEQ ID NO: 3498) | |
| 3499 | ATTTTAAATACTCACATTTTTAAGCAAACCAGGA | RTT |
| | T (SEQ ID NO: 3499) | |
| 3500 | AATTTTAAATACTCACATTTTTAAGCAAACCAGG | RTT |
| | AT (SEQ ID NO: 3500) | |
| 3501 | AAATTTTAAATACTCACATTTTTAAGCAAACCAG | RTT |
| | GAT (SEQ ID NO: 3501) | |
| 3502 | TAAATTTTAAATACTCACATTTTTAAGCAAACCA | RTT |
| | GGAT (SEQ ID NO: 3502) | |
| 3503 | ATAAATTTTAAATACTCACATTTTTAAGCAAACC | RTT |
| | AGGAT (SEQ ID NO: 3503) | |
| 3504 | GATAAATTTTAAATACTCACATTTTTAAGCAAAC | RTT |
| | CAGGAT (SEQ ID NO: 3504) | |
| 3505 | AAAGTTAAACATCTCTTCTG (SEQ ID NO: 3505) | PE3 ngRNA spacer (GAGG PAM) |
| 2560 | ACTCACATTTTTAAGCAAAC (SEQ ID NO: 2560) | PE3b ngRNA spacer (CAGG PAM) |
| 3506 | AGAAAAGTTAAACATCTCTT (SEQ ID NO: 3506) | PE3 ngRNA spacer (CTGG PAM) |
| 3507 | TCCATTTACTCTTTTTGCTG (SEQ ID NO: 3507) | PE3 ngRNA spacer (ATGG PAM) |

TABLE 52

| Sequence | | |
|---|---|---|
| Number | Sequence | Description |
| 3508 | AGCATTAAACATTTCAA (SEQ ID NO: 3508) | PEgRNA spacer (AAG PAM) |
| 3509 | CAGCATTAAACATTTCAA (SEQ ID NO: 3509) | PEgRNA spacer (AAG PAM) |
| 3510 | TCAGCATTAAACATTTCAA (SEQ ID NO: 3510) | PEgRNA spacer (AAG PAM) |
| 2672 | TTCAGCATTAAACATTTCAA (SEQ ID NO: 2672) | PEgRNA spacer (AAG PAM) |
| 3511 | ATTCAGCATTAAACATTTCAA (SEQ ID NO: 3511) | PEgRNA spacer (AAG PAM) |
| 3512 | CATTCAGCATTAAACATTTCAA (SEQ ID NO: 3512) | PEgRNA spacer (AAG PAM) |
| 3513 | AAATG | PBS |
| 3514 | AAATGT | PBS |
| 3515 | AAATGTT | PBS |
| 3516 | AAATGTTT | PBS |
| 3517 | AAATGTTTA | PBS |
| 3518 | AAATGTTTAA (SEQ ID NO: 3518) | PBS |
| 3519 | AAATGTTTAAT (SEQ ID NO: 3519) | PBS |
| 3520 | AAATGTTTAATG (SEQ ID NO: 3520) | PBS |
| 3521 | AAATGTTTAATGC (SEQ ID NO: 3521) | PBS |
| 3522 | AAATGTTTAATGCT (SEQ ID NO: 3522) | PBS |
| 3523 | AAATGTTTAATGCTG (SEQ ID NO: 3523) | PBS |
| 3524 | AAATGTTTAATGCTGA (SEQ ID NO: 3524) | PBS |
| 3525 | AAATGTTTAATGCTGAA (SEQ ID NO: 3525) | PBS |
| 3526 | AAATGTTTAATGCTGAAT (SEQ ID NO: 3526) | PBS |
| 3527 | AAATGTTTAATGCTGAATG (SEQ ID NO: 3527) | PBS |
| 3528 | AGTATTTAAAATTTATCACTTTTG (SEQ ID NO: | RTT |
| | 3528) | |
| 3529 | GAGTATTTAAAATTTATCACTTTTG (SEQ ID NO: | RTT |
| | 3529) | |
| 3530 | TGAGTATTTAAAATTTATCACTTTTG (SEQ ID NO: | RTT |
| | 3530) | |
| 3531 | GTGAGTATTTAAAATTTATCACTTTTG (SEQ ID | RTT |
| | NO: 3531) | |
| 3532 | TGTGAGTATTTAAAATTTATCACTTTTG (SEQ ID | RTT |
| | NO: 3532) | |
| 3533 | ATGTGAGTATTTAAAATTTATCACTTTTG (SEQ ID | RTT |
| | NO: 3533) | |
| 3534 | AATGTGAGTATTTAAAATTTATCACTTTTG (SEQ | RTT |
| | ID NO: 3534) | |
| 3535 | AAATGTGAGTATTTAAAATTTATCACTTTTG (SEQ | RTT |
| | ID NO: 3535) | |
| 3536 | AAAATGTGAGTATTTAAAATTTATCACTTTTG | RTT |
| | (SEQ ID NO: 3536) | |
| 3537 | AAAAATGTGAGTATTTAAAATTTATCACTTTTG | RTT |
| | (SEQ ID NO: 3537) | |
| 3538 | TAAAAATGTGAGTATTTAAAATTTATCACTTTTG | RTT |
| | (SEQ ID NO: 3538) | |
| 3539 | TTAAAAATGTGAGTATTTAAAATTTATCACTTTT | RTT |
| | G (SEQ ID NO: 3539) | |
| 3540 | CTTAAAAATGTGAGTATTTAAAATTTATCACTTT | RTT |
| | TG (SEQ ID NO: 3540) | |
| 3541 | GCTTAAAAATGTGAGTATTTAAAATTTATCACTT | RTT |
| | TTG (SEQ ID NO: 3541) | |
| 3542 | TGCTTAAAAATGTGAGTATTTAAAATTTATCACT | RTT |
| | TTTG (SEQ ID NO: 3542) | |
| 3543 | TTGCTTAAAAATGTGAGTATTTAAAATTTATCAC | RTT |
| | TTTTG (SEQ ID NO: 3543) | |
| 3544 | TTTGCTTAAAAATGTGAGTATTTAAAATTTATCA | RTT |
| | CTTTTG (SEQ ID NO: 3544) | |
| 3550 | GAGTATTTAAAATTTATCATTGTTG (SEQ ID NO: | RTT (AAG-to-CAA for intronic PEgRNA |
| | 3550) | spacer PAM silencing edit) |
| 3551 | TGAGTATTTAAAATTTATCATTGTTG (SEQ ID NO: | RTT (AAG-to-CAA for intronic PEgRNA |
| | 3551) | spacer PAM silencing edit) |
| 3552 | GTGAGTATTTAAAATTTATCATTGTTG (SEQ ID | RTT (AAG-to-CAA for intronic PEgRNA |
| | NO: 3552) | spacer PAM silencing edit) |
| 3553 | TGTGAGTATTTAAAATTTATCATTGTTG (SEQ ID | RTT (AAG-to-CAA for intronic PEgRNA |
| | NO: 3553) | spacer PAM silencing edit) |
| 3554 | ATGTGAGTATTTAAAATTTATCATTGTTG (SEQ ID | RTT (AAG-to-CAA for intronic PEgRNA |
| | NO: 3554) | spacer PAM silencing edit) |
| 3555 | AATGTGAGTATTTAAAATTTATCATTGTTG (SEQ | RTT (AAG-to-CAA for intronic PEgRNA |
| | ID NO: 3555) | spacer PAM silencing edit) |
| 3556 | AAATGTGAGTATTTAAAATTTATCATTGTTG (SEQ | RTT (AAG-to-CAA for intronic PEgRNA |
| | ID NO: 3556) | spacer PAM silencing edit) |
| 3557 | AAAATGTGAGTATTTAAAATTTATCATTGTTG | RTT (AAG-to-CAA for intronic PEgRNA |
| | (SEQ ID NO: 3557) | spacer PAM silencing edit) |
| 3558 | AAAAATGTGAGTATTTAAAATTTATCATTGTTG | RTT (AAG-to-CAA for intronic PEgRNA |
| | (SEQ ID NO: 3558) | spacer PAM silencing edit) |
| 3559 | TAAAAATGTGAGTATTTAAAATTTATCATTGTTG | RTT (AAG-to-CAA for intronic PEgRNA |
| | (SEQ ID NO: 3559) | spacer PAM silencing edit) |
| 3560 | TTAAAAATGTGAGTATTTAAAATTTATCATTGTT | RTT (AAG-to-CAA for intronic PEgRNA |
| | G (SEQ ID NO: 3560) | spacer PAM silencing edit) |
| 3561 | CTTAAAAATGTGAGTATTTAAAATTTATCATTGT | RTT (AAG-to-CAA for intronic PEgRNA |
| | TG (SEQ ID NO: 3561) | spacer PAM silencing edit) |
| 3562 | GCTTAAAAATGTGAGTATTTAAAATTTATCATTG | RTT (AAG-to-CAA for intronic PEgRNA |
| | TTG (SEQ ID NO: 3562) | spacer PAM silencing edit) |
| 3563 | TGCTTAAAAATGTGAGTATTTAAAATTTATCATT | RTT (AAG-to-CAA for intronic PEgRNA |
| | GTTG (SEQ ID NO: 3563) | spacer PAM silencing edit) |
| 3564 | TTGCTTAAAAATGTGAGTATTTAAAATTTATCAT | RTT (AAG-to-CAA for intronic PEgRNA |
| | TGTTG (SEQ ID NO: 3564) | spacer PAM silencing edit) |
| 3565 | TTTGCTTAAAAATGTGAGTATTTAAAATTTATCA | RTT (AAG-to-CAA for intronic PEgRNA |
| | TTGTTG (SEQ ID NO: 3565) | spacer PAM silencing edit) |
| 3545 | AGAAGTTGCTCTTTTCACTC (SEQ ID NO: 3545) | PE3 ngRNA spacer (AAG PAM) |
| 3546 | AGCACTCAGATTTGATAAAG (SEQ ID NO: 3546) | PE3 ngRNA spacer (AAG PAM) |
| 2634 | AGGTCTTGGGTATGCACCTA (SEQ ID NO: 2634) | PE3 ngRNA spacer (TAG PAM) |
| 3547 | TCTCATATACTTTCAGCACT (SEQ ID NO: 3547) | PE3 ngRNA spacer (CAG PAM) |
| 3548 | TGTATTATCTCATATACTTT (SEQ ID NO: 3548) | PE3 ngRNA spacer (CAG PAM) |
| 3549 | TTCAGCACTCAGATTTGATA (SEQ ID NO: 3549) | PE3 ngRNA spacer (AAG PAM) |

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, tale magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., an FANCC gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., an FANCC gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g. the target FANCC gene, upon the contacting of the PE composition with the target gene. In some embodiments, the DNA binding domain of the PE associates with the PEgRNA. In some embodiments, the PE binds the target gene, e.g. an FANCC gene, directed by the PEgRNA. Accordingly, in some embodiments, the contacting of the target gene result in binding of a DNA binding domain of a prime editor of the target FANCC gene directed by the PEgRNA.

In some embodiments, contacting the target gene with the prime editing composition results in a nick in an edit strand of the target gene, e.g. an FANCC gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene, e.g., an FANCC gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g. an FANCC gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene, e.g. a FANCC gene, is in a cell. Accordingly, also provided herein are methods of modifying a cell, such as a human cell, a human primary cell, a human iPSC-derived cell, and HSPC.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery. In some embodiments, the polynucleotide is a DNA polynucleotide. In some embodiments, the polynucleotide is a RNA polynucleotide, e.g., mRNA polynucleotide.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell.

In some embodiments, the cell is a eukaryotic cell. In some embodiments, the cell is a mammalian cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a hepatocyte. In some embodiments, the cell is a human HSPC. In some embodiments, the cell is a progenitor cell. In some embodiments, the cell is a stem cell. in some embodiments, the cell is an induced pluripotent stem cell. In some embodiments, the cell is an embryonic stem cell. In some embodiments, the cell is a hematopoietic progenitor cell. In some embodiments, the cell is a hematopoietic stem cell. In some embodiments, the cell is a hematopoietic stem or progenitor cell (HSPC). In some embodiments, the cell is a fibroblast. In some embodiments, the cell is a CD34+ cell. In some embodiments, the cell is a hematopoietic stem cell (HSC). In some embodiments, the cell is a hematopoietic progenitor cell (HPC). In some embodiments, the cell is a human HSPC. In some embodiments, the cell is a human HPC. In some embodiments, the cell is a human HSC. In some embodiments, the cell is a long term (LT)-HSC. In some embodiments, the cell is a short-term(ST)-HSC. In some embodiments, the cell is a myeloid progenitor cell. In some embodiments, the cell is a lymphoid progenitor cell. In some embodiments, the cell is a granulocyte monocyte progenitor cell. In some embodiments, the cell is a megakaryocyte erythroid progenitor cell.

In some embodiments, the cell is a human progenitor cell. In some embodiments, the cell is a human stem cell. in some embodiments, the cell is an induced human pluripotent stem cell. In some embodiments, the cell is a human embryonic stem cell. In some embodiments, the cell is a human fibroblast. In some embodiments, the cell is a primary cell. In some embodiments, the cell is a human primary cell. In some embodiments, the cell is a differentiated cell.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a FANCC gene within the genome of a cell) to a prime editing composition. In some embodiments, editing efficiency of the prime editing compositions and method described herein can be measured by calculating the percentage of edited target genes in a population of cells introduced with the prime editing composition. In some embodiments, the editing efficiency is determined after 5, 6, 7, 8, 9, 10, 11, 12, 13, 14, 15, or 16 weeks of exposing a target (e.g., a FANCC gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control. In some embodiments, editing efficiency of the prime editing compositions and method described herein can be measured by calculating the percentage of edited target genes in a population of cells after in vivo engraftment of the edited cells. In some embodiments, the editing efficiency is determined after 5, 6, 7, 8, 9, 10, 11, 12, 13, 14, 15, or 16 weeks of engraftment. In some embodiments, the editing efficiency is determined after 8 or 16 weeks of engraftment. In some embodiments, prime editing is able to maintain in edited cells at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more than 95% of editing efficiency after 8 or 16 weeks post engraftment.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a target cell, e.g., a human HSPC(as measured in a population of cells) relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a target cell (e.g., HSPC) relative to a corresponding control cell HSPC. In some embodiments, the target cell is a human cell. In some embodiments, the HSPC is a human HSPC.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the prime editing methods disclosed herein can have an indel frequency of less than 30%, less than 20%, less than 10%, less than 9%, less than 8%, less than 7%, less than 6%, less than 5%, less than 4%, less than 3%, less than 2%, less than 1.5%, or less than 1%.

In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a FANCC gene within the genome of a cell) to a prime editing composition.

In some embodiments, the prime editing compositions provided herein are capable of incorporating one or more intended nucleotide edits efficiently without generating a significant proportion of indels in a population of target cells as compared to a population of corresponding control cells. In some embodiments, the population of target cell comprises a population of human primary cells, human iPSCs, or human HSPCs.

In some embodiments, the prime editing methods disclosed herein have an editing efficiency of at least about 1% and an indel frequency of less than 10% in a population of target cells, e.g., a population of human HSPCs. In some embodiments, the prime editing methods disclosed herein have an editing efficiency of at least about 1% and an indel frequency of less than 7.5% in a population of target cells, e.g., a population of human HSPCs. In some embodiments, the prime editing methods disclosed herein have an editing efficiency of at least about 1% and an indel frequency of less than 5% in a population of target cells, e.g., a population of human HSPCs. In some embodiments, the prime editing methods disclosed herein have an editing efficiency of at least about 1% and an indel frequency of less than 2.5% in a population of target cells, e.g., a population of human HSPCs. In some embodiments, the prime editing methods disclosed herein have an editing efficiency of at least about 1% and an indel frequency of less than 1% in a population of target cells, e.g., a population of human HSPCs. In some embodiments, the prime editing methods disclosed herein have an editing efficiency of at least about 1% and an indel frequency of less than 0.5% in a population of target cells, e.g., a population of human HSPCs. In some embodiments, the prime editing methods disclosed herein have an editing efficiency of at least about 1% and an indel frequency of less than 0.1% in a population of target cells, e.g., a population of human HSPCs.

In some embodiments, the prime editing methods disclosed herein have an editing efficiency of at least about 5% and an indel frequency of less than 10% in a population of target cells, e.g., a population of human HSPCs. In some embodiments, the prime editing methods disclosed herein have an editing efficiency of at least about 5% and an indel frequency of less than 7.5% in a population of target cells, e.g., a population of human HSPCs. In some embodiments, the prime editing methods disclosed herein have an editing efficiency of at least about 5% and an indel frequency of less than 5% in a population of target cells, e.g., a population of human HSPCs. In some embodiments, the prime editing methods disclosed herein have an editing efficiency of at least about 5% and an indel frequency of less than 2.5% in a population of target cells, e.g., a population of human HSPCs. In some embodiments, the prime editing methods disclosed herein have an editing efficiency of at least about 5% and an indel frequency of less than 1% in a population of target cells, e.g., a population of human HSPCs. In some embodiments, the prime editing methods disclosed herein have an editing efficiency of at least about 5% and an indel frequency of less than 0.5% in a population of target cells, e.g., a population of human HSPCs. In some embodiments, the prime editing methods disclosed herein have an editing efficiency of at least about 5% and an indel frequency of less than 0.1% in a population of target cells, e.g., a population of human HSPCs.

In some embodiments, the prime editing methods disclosed herein have an editing efficiency of at least about 7.5% and an indel frequency of less than 10% in a population of target cells, e.g., a population of human HSPCs. In some embodiments, the prime editing methods disclosed herein have an editing efficiency of at least about 7.5% and an indel frequency of less than 7.5% in a population of target cells, e.g., a population of human HSPCs. In some embodiments, the prime editing methods disclosed herein have an editing efficiency of at least about 7.5% and an indel frequency of less than 5% in a population of target cells, e.g., a population of human HSPCs. In some embodiments, the prime editing methods disclosed herein have an editing efficiency of at least about 7.5% and an indel frequency of less than 2.5% in a population of target cells, e.g., a population of human HSPCs. In some embodiments, the prime editing methods disclosed herein have an editing efficiency of at least about 7.5% and an indel frequency of less than 1% in a population of target cells, e.g., a population of human HSPCs. In some embodiments, the prime editing methods disclosed herein have an editing efficiency of at least about 7.5% and an indel frequency of less than 0.5% in a population of target cells, e.g., a population of human HSPCs. In some embodiments, the prime editing methods disclosed herein have an editing efficiency of at least about 7.5% and an indel frequency of less than 0.1% in a population of target cells, e.g., a population of human HSPCs.

In some embodiments, the prime editing methods disclosed herein have an editing efficiency of at least about 10% and an indel frequency of less than 10% in a population of target cells, e.g., a population of human HSPCs. In some embodiments, the prime editing methods disclosed herein have an editing efficiency of at least about 10% and an indel frequency of less than 7.5% in a population of target cells, e.g., a population of human HSPCs. In some embodiments, the prime editing methods disclosed herein have an editing efficiency of at least about 10% and an indel frequency of less than 5% in a population of target cells, e.g., a population of human HSPCs. In some embodiments, the prime editing methods disclosed herein have an editing efficiency of at least about 10% and an indel frequency of less than 2.5% in a population of target cells, e.g., a population of human HSPCs. In some embodiments, the prime editing methods disclosed herein have an editing efficiency of at least about 10% and an indel frequency of less than 1% in a population of target cells, e.g., a population of human HSPCs. In some embodiments, the prime editing methods disclosed herein have an editing efficiency of at least about 10% and an indel frequency of less than 0.5% in a population of target cells, e.g., a population of human HSPCs. In some embodiments, the prime editing methods disclosed herein have an editing efficiency of at least about 10% and an indel frequency of less than 0.1% in a population of target cells, e.g., a population of human HSPCs.

In some embodiments, the prime editing methods disclosed herein have an editing efficiency of at least about 15% and an indel frequency of less than 10% in a population of target cells, e.g., a population of human HSPCs. In some embodiments, the prime editing methods disclosed herein have an editing efficiency of at least about 15% and an indel frequency of less than 7.5% in a population of target cells, e.g., a population of human HSPCs. In some embodiments, the prime editing methods disclosed herein have an editing efficiency of at least about 15% and an indel frequency of less than 5% in a population of target cells, e.g., a population of human HSPCs. In some embodiments, the prime editing methods disclosed herein have an editing efficiency of at least about 15% and an indel frequency of less than 2.5% in a population of target cells, e.g., a population of human HSPCs. In some embodiments, the prime editing methods disclosed herein have an editing efficiency of at least about 15% and an indel frequency of less than 1% in a population of target cells, e.g., a population of human HSPCs. In some embodiments, the prime editing methods disclosed herein have an editing efficiency of at least about 15% and an indel frequency of less than 0.5% in a population of target cells, e.g., a population of human HSPCs. In some embodiments, the prime editing methods disclosed herein have an editing efficiency of at least about 15% and an indel frequency of less than 0.1% in a population of target cells, e.g., a population of human HSPCs.

In some embodiments, the prime editing methods disclosed herein have an editing efficiency of at least about 20% and an indel frequency of less than 10% in a population of target cells, e.g., a population of human HSPCs. In some embodiments, the prime editing methods disclosed herein have an editing efficiency of at least about 20% and an indel frequency of less than 7.5% in a population of target cells, e.g., a population of human HSPCs. In some embodiments, the prime editing methods disclosed herein have an editing efficiency of at least about 20% and an indel frequency of less than 5% in a population of target cells, e.g., a population of human HSPCs. In some embodiments, the prime editing methods disclosed herein have an editing efficiency of at least about 20% and an indel frequency of less than 2.5% in a population of target cells, e.g., a population of human HSPCs. In some embodiments, the prime editing methods disclosed herein have an editing efficiency of at least about 20% and an indel frequency of less than 1% in a population of target cells, e.g., a population of human HSPCs. In some embodiments, the prime editing methods disclosed herein have an editing efficiency of at least about 20% and an indel frequency of less than 0.5% in a population of target cells, e.g., a population of human HSPCs. In some embodiments, the prime editing methods disclosed herein have an editing efficiency of at least about 20% and an indel frequency of less than 0.1% in a population of target cells, e.g., a population of human HSPCs.

In some embodiments, the prime editing methods disclosed herein have an editing efficiency of at least about 30% and an indel frequency of less than 10% in a population of target cells, e.g., a population of human HSPCs. In some embodiments, the prime editing methods disclosed herein have an editing efficiency of at least about 30% and an indel frequency of less than 7.5% in a population of target cells, e.g., a population of human HSPCs. In some embodiments, the prime editing methods disclosed herein have an editing efficiency of at least about 30% and an indel frequency of less than 5% in a population of target cells, e.g., a population of human HSPCs. In some embodiments, the prime editing methods disclosed herein have an editing efficiency of at least about 30% and an indel frequency of less than 2.5% in a population of target cells, e.g., a population of human HSPCs. In some embodiments, the prime editing methods disclosed herein have an editing efficiency of at least about 30% and an indel frequency of less than 1% in a population of target cells, e.g., a population of human HSPCs. In some embodiments, the prime editing methods disclosed herein have an editing efficiency of at least about 30% and an indel frequency of less than 0.5% in a population of target cells, e.g., a population of human HSPCs. In some embodiments, the prime editing methods disclosed herein have an editing efficiency of at least about 30% and an indel frequency of less than 0.1% in a population of target cells, e.g., a population of human HSPCs.

In some embodiments, the prime editing methods disclosed herein have an editing efficiency of at least about 40% and an indel frequency of less than 10% in a population of target cells, e.g., a population of human HSPCs. In some embodiments, the prime editing methods disclosed herein have an editing efficiency of at least about 40% and an indel frequency of less than 7.5% in a population of target cells, e.g., a population of human HSPCs. In some embodiments, the prime editing methods disclosed herein have an editing efficiency of at least about 40% and an indel frequency of less than 5% in a population of target cells, e.g., a population of human HSPCs. In some embodiments, the prime editing methods disclosed herein have an editing efficiency of at least about 40% and an indel frequency of less than 2.5% in a population of target cells, e.g., a population of human HSPCs. In some embodiments, the prime editing methods disclosed herein have an editing efficiency of at least about 40% and an indel frequency of less than 1% in a population of target cells, e.g., a population of human HSPCs. In some embodiments, the prime editing methods disclosed herein have an editing efficiency of at least about 40% and an indel frequency of less than 0.5% in a population of target cells, e.g., a population of human HSPCs. In some embodiments, the prime editing methods disclosed herein have an editing efficiency of at least about 40% and an indel frequency of less than 0.1% in a population of target cells, e.g., a population of human HSPCs.

In some embodiments, the prime editing methods disclosed herein have an editing efficiency of at least about 50% and an indel frequency of less than 10% in a population of target cells, e.g., a population of human HSPCs. In some embodiments, the prime editing methods disclosed herein have an editing efficiency of at least about 50% and an indel frequency of less than 7.5% in a population of target cells, e.g., a population of human HSPCs. In some embodiments, the prime editing methods disclosed herein have an editing efficiency of at least about 50% and an indel frequency of less than 5% in a population of target cells, e.g., a population of human HSPCs. In some embodiments, the prime editing methods disclosed herein have an editing efficiency of at least about 50% and an indel frequency of less than 2.5% in a population of target cells, e.g., a population of human HSPCs. In some embodiments, the prime editing methods disclosed herein have an editing efficiency of at least about 50% and an indel frequency of less than 1% in a population of target cells, e.g., a population of human HSPCs. In some embodiments, the prime editing methods disclosed herein have an editing efficiency of at least about 50% and an indel frequency of less than 0.5% in a population of target cells, e.g., a population of human HSPCs. In some embodiments, the prime editing methods disclosed herein have an editing efficiency of at least about 50% and an indel frequency of less than 0.1% in a population of target cells, e.g., a population of human HSPCs.

In some embodiments, the prime editing methods disclosed herein have an editing efficiency of at least about 60% and an indel frequency of less than 10% in a population of target cells, e.g., a population of human HSPCs. In some embodiments, the prime editing methods disclosed herein have an editing efficiency of at least about 60% and an indel frequency of less than 7.5% in a population of target cells, e.g., a population of human HSPCs. In some embodiments, the prime editing methods disclosed herein have an editing efficiency of at least about 60% and an indel frequency of less than 5% in a population of target cells, e.g., a population of human HSPCs. In some embodiments, the prime editing methods disclosed herein have an editing efficiency of at least about 60% and an indel frequency of less than 2.5% in a population of target cells, e.g., a population of human HSPCs. In some embodiments, the prime editing methods disclosed herein have an editing efficiency of at least about 60% and an indel frequency of less than 1% in a population of target cells, e.g., a population of human HSPCs. In some embodiments, the prime editing methods disclosed herein have an editing efficiency of at least about 60% and an indel frequency of less than 0.5% in a population of target cells, e.g., a population of human HSPCs. In some embodiments, the prime editing methods disclosed herein have an editing efficiency of at least about 60% and an indel frequency of less than 0.1% in a population of target cells, e.g., a population of human HSPCs.

In some embodiments, the prime editing methods disclosed herein have an editing efficiency of at least about 70% and an indel frequency of less than 10% in a population of target cells, e.g., a population of human HSPCs. In some embodiments, the prime editing methods disclosed herein have an editing efficiency of at least about 70% and an indel frequency of less than 7.5% in a population of target cells, e.g., a population of human HSPCs. In some embodiments, the prime editing methods disclosed herein have an editing efficiency of at least about 70% and an indel frequency of less than 5% in a population of target cells, e.g., a population of human HSPCs. In some embodiments, the prime editing methods disclosed herein have an editing efficiency of at least about 70% and an indel frequency of less than 2.5% in a population of target cells, e.g., a population of human HSPCs. In some embodiments, the prime editing methods disclosed herein have an editing efficiency of at least about 70% and an indel frequency of less than 1% in a population of target cells, e.g., a population of human HSPCs. In some embodiments, the prime editing methods disclosed herein have an editing efficiency of at least about 70% and an indel frequency of less than 0.5% in a population of target cells, e.g., a population of human HSPCs. In some embodiments, the prime editing methods disclosed herein have an editing efficiency of at least about 70% and an indel frequency of less than 0.1% in a population of target cells, e.g., a population of human HSPCs.

In some embodiments, the prime editing methods disclosed herein have an editing efficiency of at least about 80% and an indel frequency of less than 10% in a population of target cells, e.g., a population of human HSPCs. In some embodiments, the prime editing methods disclosed herein have an editing efficiency of at least about 80% and an indel frequency of less than 7.5% in a population of target cells, e.g., a population of human HSPCs. In some embodiments, the prime editing methods disclosed herein have an editing efficiency of at least about 80% and an indel frequency of less than 5% in a population of target cells, e.g., a population of human HSPCs. In some embodiments, the prime editing methods disclosed herein have an editing efficiency of at least about 80% and an indel frequency of less than 2.5% in a population of target cells, e.g., a population of human HSPCs. In some embodiments, the prime editing methods disclosed herein have an editing efficiency of at least about 80% and an indel frequency of less than 1% in a population of target cells, e.g., a population of human HSPCs. In some embodiments, the prime editing methods disclosed herein have an editing efficiency of at least about 80% and an indel frequency of less than 0.5% in a population of target cells, e.g., a population of human HSPCs. In some embodiments, the prime editing methods disclosed herein have an editing efficiency of at least about 80% and an indel frequency of less than 0.1% in a population of target cells, e.g., a population of human HSPCs.

In some embodiments, the prime editing methods disclosed herein have an editing efficiency of at least about 90% and an indel frequency of less than 10% in a population of target cells, e.g., a population of human HSPCs. In some embodiments, the prime editing methods disclosed herein have an editing efficiency of at least about 90% and an indel frequency of less than 7.5% in a population of target cells, e.g., a population of human HSPCs. In some embodiments, the prime editing methods disclosed herein have an editing efficiency of at least about 90% and an indel frequency of less than 5% in a population of target cells, e.g., a population of human HSPCs. In some embodiments, the prime editing methods disclosed herein have an editing efficiency of at least about 90% and an indel frequency of less than 2.5% in a population of target cells, e.g., a population of human HSPCs. In some embodiments, the prime editing methods disclosed herein have an editing efficiency of at least about 90% and an indel frequency of less than 1% in a population of target cells, e.g., a population of human HSPCs. In some embodiments, the prime editing methods disclosed herein have an editing efficiency of at least about 90% and an indel frequency of less than 0.5% in a population of target cells, e.g., a population of human HSPCs. In some embodiments, the prime editing methods disclosed herein have an editing efficiency of at least about 90% and an indel frequency of less than 0.1% in a population of target cells, e.g., a population of human HSPCs.

In some embodiments, the prime editing methods disclosed herein have an editing efficiency of at least about 95% and an indel frequency of less than 10% in a population of target cells, e.g., a population of human HSPCs. In some embodiments, the prime editing methods disclosed herein have an editing efficiency of at least about 95% and an indel frequency of less than 7.5% in a population of target cells, e.g., a population of human HSPCs. In some embodiments, the prime editing methods disclosed herein have an editing efficiency of at least about 95% and an indel frequency of less than 5% in a population of target cells, e.g., a population of human HSPCs. In some embodiments, the prime editing methods disclosed herein have an editing efficiency of at least about 95% and an indel frequency of less than 2.5% in a population of target cells, e.g., a population of human HSPCs. In some embodiments, the prime editing methods disclosed herein have an editing efficiency of at least about 95% and an indel frequency of less than 1% in a population of target cells, e.g., a population of human HSPCs. In some embodiments, the prime editing methods disclosed herein have an editing efficiency of at least about 95% and an indel frequency of less than 0.5% in a population of target cells, e.g., a population of human HSPCs. In some embodiments, the prime editing methods disclosed herein have an editing efficiency of at least about 95% and an indel frequency of less than 0.1% in a population of target cells, e.g., a population of human HSPCs.

In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a FANCC gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a FANCC gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing methods described herein result in less than 10%, less than 9%, less than 8%, less than 7%, less than 6%, less than 5%, less than 4%, less than 3%, less than 2%, less than 1%, or less than 0.5% large deletion in edited cells. In some embodiments, the prime editing methods described herein result in less than 4% large deletion in edited cells. In some embodiments, the prime editing methods described herein result in less than 3% large deletion in edited cells. In some embodiments, the prime editing methods described herein result in less than 2% large deletion in edited cells. In some embodiments, the prime editing methods described herein result in less than 1% large deletion in edited cells. In some embodiments, the prime editing methods described herein does not result in detectable level of large deletion in edited cells.

In some embodiments, the prime editing compositions (e.g., PEgRNAs and prime editors as described herein) and prime editing methods disclosed herein can be used to edit a target FANCC gene. In some embodiments, the target FANCC gene comprises a mutation compared to a wild type FANCC gene. In some embodiments, the mutation is associated with Fanconi anemia. In some embodiments, the target FANCC gene comprises an editing target sequence that contains the mutation associated with Fanconi anemia. In some embodiments, the mutation is in a coding region of the target FANCC gene. In some embodiments, the mutation is in an exon of the target FANCC gene. In some embodiments, the mutation is in exon 1 of the FANCC gene as compared to a wild type FANCC gene. In some embodiments, the editing target sequence comprises a mutation in an intron of the FANCC gene as compared to a wild type FANCC gene. In some embodiments, the mutation is in intron 4 of the FANCC gene as compared to a wild type FANCC gene.

Unless otherwise indicated, references to nucleotide positions in human chromosomes are as set forth in human genome assembly consortium Human build 38 (GRCh38), GenBank accession GCF_000001405.38.

In some embodiments, the editing target sequence comprises a mutation in intron 4 of the FANCC gene compared to a wild type FANCC gene. In some embodiments, the editing target sequence comprises a mutation that is located between positions 95,171,933 and 95,172,133 of human chromosome 9. In some embodiments, the editing target sequence comprises a mutation that encodes a nucleotide substitution compared to a wild type FANCC gene as set forth in SEQ ID NO: 3809. In some embodiments, the editing target sequence comprises a nucleotide transversion relative to a wild type FANCC gene set forth as SEQ ID NO: 3809. In some embodiments, the editing target sequence comprises an A to T transversion at position 95,172,033 in human chromosome 9 (a c.456+4A->T (IVS4+4A>T) mutation) as compared to a wild type FANCC gene.

In some embodiments, the editing target sequence comprises a mutation in exon 1 of the FANCC gene as compared to a wild type FANCC gene. In some embodiments, the editing target sequence comprises a mutation that is located between positions 95,249,125 and 95,249,325 of human chromosome 9. In some embodiments, the editing target sequence comprises a mutation that results in a frameshift in a transcript encoded by the FANCC gene as compared to a transcript encoded by a wild type FANCC gene. In some embodiments, the editing target sequence comprises a mutation that is a deletion compared to a wild type FANCC gene set forth as SEQ ID NO: 3809. In some embodiments, the editing target sequence comprises a deletion of a nucleotide guanine at position 95,249,225 in human chromosome 9 (a c.67del or 322delG mutation) as compared to a wild type FANCC gene.

In some embodiments, the prime editing method comprises contacting a target FANCC gene with a prime editing composition comprising a prime editor, a PEgRNA, and/or a ngRNA. In some embodiments, contacting the target FANCC gene with the prime editing composition results in incorporation of one or more intended nucleotide edits in the target FANCC gene. In some embodiments, the incorporation is in a region of the target FANCC gene that corresponds to an editing target sequence in the FANCC gene. In some embodiments, the one or more intended nucleotide edits comprises a single nucleotide substitution, an insertion, a deletion, or any combination thereof, compared to the endogenous sequence of the target FANCC gene. In some embodiments, incorporation of the one or more intended nucleotide edits results in replacement of one or more mutations with the corresponding sequence that encodes a wild type FANCC polypeptide. In some embodiments, incorporation of the one or more intended nucleotide edits results in replacement of the one or more mutations with the corresponding sequence in a wild type FANCC gene. In some embodiments, incorporation of the one more intended nucleotide edits results in correction of a mutation in the target FANCC gene. In some embodiments, the target FANCC gene comprises an editing template sequence that contains the mutation. In some embodiments, contacting the target FANCC gene with the prime editing composition results in incorporation of one or more intended nucleotide edits in the target FANCC gene, which corrects the mutation in the editing target sequence (or a double stranded region comprising the editing target sequence and the complementary sequence to the editing target sequence on a target strand) in the target FANCC gene.

In some embodiments, incorporation of the one more intended nucleotide edits results in correction of a mutation in intron 4 of the target FANCC gene as compared to a wild type FANCC gene. In some embodiments, incorporation of the one more intended nucleotide edits results in correction of a mutation located between 95,171,933 and 95,172,133 of human chromosome 9. In some embodiments, incorporation of the one more intended nucleotide edits results in correction of a IVS4+4 A->T mutation. In some embodiments, incorporation of the one or more intended nucleotide edits results in a T to A nucleotide substitution (on the sense strand) in the FANCC gene at a position corresponding to position 95,172,033 of human chromosome 9. In some embodiments, incorporation of the one more intended nucleotide edits results in correction of a FANCC gene sequence that encodes a FANCC transcript that lacks exon 4 compared to a wild type FANCC mRNA, and restores wild type expression and function of the FANCC protein.

In some embodiments, incorporation of the one more intended nucleotide edits results in correction of a mutation in exon 1 of the target FANCC gene as compared to a wild type FANCC gene. In some embodiments, incorporation of the one more intended nucleotide edits results in correction of a mutation located between positions 95,249,125 and 95,249,325 of human chromosome 9. In some embodiments, incorporation of the one more intended nucleotide edits results in correction of a mutation that encodes a c.67del (322delG) nucleotide mutation. In some embodiments, incorporation of the one more intended nucleotide edits results in a nucleotide insertion at a position corresponding to position 95,248,225 in human chromosome 9. In some embodiments, incorporation of the one more intended nucleotide edits results in an insertion of nucleotide Cytidine (on the sense strand) in the FANCC gene. In some embodiments, incorporation of the one more intended nucleotide edits results in correction of an FANCC gene sequence that encodes a c.67del (322delG) nucleotide mutation, and restores wild type expression and function of the FANCC protein.

In some embodiments, the target FANCC gene is in a target cell. Accordingly, in one aspect provided herein is a method of editing a target cell comprising a target FANCC gene that encodes a polypeptide that comprises one or more mutations relative to a wild type FANCC gene. In some embodiments, the methods of the present disclosure comprise introducing a prime editing composition comprising a PEgRNA, a prime editor polypeptide, and/or a ngRNA into the target cell that has the target FANCC gene to edit the target FANCC gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a HSC. In some embodiments, the target cell is a human HSC. In some embodiments, the target cell is a HSPC. In some embodiments, the target cell is a CD34+ cell. In some embodiments, the target cell is a human HSPC. In some embodiments, the target cell is a human CD34+ cell. In some embodiments, the target cell is a human HSPC. In some embodiments, the target cell is a human cell. In some embodiments, the target cell is an embryonic stem cell. In some embodiments, the target cell is a primary human hair cell derived from an induced human pluripotent stem cell (iPSC). In some embodiments, the target cell is a differentiated cell derived from an induced human pluripotent stem cell (iPSC). In some embodiments, the cell is a differentiated cell derived from a hematopoietic stem cell. In some embodiments, the cell is a CD34+ cell. In some embodiments, the cell is a hematopoietic stem cell (HSC). In some embodiments, the cell is a hematopoietic progenitor cell (HPC). In some embodiments, hematopoietic stem cells and hematopoietic progenitor cells are referred to as hematopoietic stem or progenitor cells (HSPCs). In some embodiments, the cell is a human HSC. In some embodiments, the cell is a human HPC. In some embodiments, the cell is a human HSPC. In some embodiments, the cell is a long term (LT)-HSC. In some embodiments, the cell is a short-term(ST)-HSC. In some embodiments, the cell is a myeloid progenitor cell. In some embodiments, the cell is a multipotent progenitor cell (MPP). In some embodiments, the cell is a lymphoid progenitor cell. In some embodiments, the cell is a granulocyte monocyte progenitor cell. In some embodiments, the cell is a megakaryocyte erythroid progenitor cell.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo. In some embodiments, the cell edited by prime editing can be differentiated into, or give rise to recovery of a population of cells. In some embodiments, the target cell is an ex vivo cell. In some embodiments, the target cell is an ex vivo cell obtained from a human subject. In some embodiments, the target cell is in a subject, e.g., a human subject.

In some embodiments, incorporation of the one or more intended nucleotide edits in the target FANCC gene that comprises one or more mutations restores wild type expression and function of the FANCC protein encoded by the FANCC gene. In some embodiments, the target FANCC gene comprises a frameshifting mutation, e.g., a c.67del (322delG) mutation compared to a wild type FANCC gene prior to incorporation of the one or more intended nucleotide edits. In some embodiments, the target FANCC gene comprises a mutation that results in aberrant splicing of the FANCC mRNA, e.g., a c.456+4A->T (IVS4+4A>T) mutation compared to a wild type FANCC gene. In some embodiments, expression and/or function of the FANCC protein may be measured when expressed in a target cell. In some embodiments, incorporation of the one or more intended nucleotide edits in the target FANCC gene comprising one or more mutations lead to a fold change in a level of FANCC gene expression, FANCC protein expression, or a combination thereof. In some embodiments, a change in the level of FANCC expression level can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target FANCC gene that comprises one or more mutations restores wild type expression of the FANCC protein by at least 10%, 20%, 30%, 40%, 50%, 60%, 70%, 80%, 90%, 95%, 99% or more as compared to wild type expression of the FANCC protein in a suitable control cell that comprises a wild type FANCC gene.

In some embodiments, a FANCC expression increase can be measured by a functional assay. In some embodiments, protein expression can be measured using a protein assay. In some embodiments, protein expression can be measured using antibody testing. In some embodiments, an antibody can comprise anti-FANCC. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

### Methods of Treating Fanconi Anemia of Complementation Group C (FA-C)

In some embodiments, provided herein are methods for treatment of a subject diagnosed with a disease associated with or caused by one or more pathogenic mutations. In some embodiments, provided herein are methods for treatment of a subject diagnosed with a disease associated with or caused by one or more pathogenic mutations that can be corrected by prime editing. In some embodiments, methods of treatment provided herein comprises editing one or more genes other than the gene that harbors the one or more pathogenic mutations. In some embodiments, provided herein are methods for treating Fanconi anemia of complementation group C (FA-C) that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the target gene in the subject. In some embodiments, administration of the prime editing composition results in correction of one or more pathogenic mutations, e.g. point mutations, insertions, or deletions, associated with Fanconi anemia of complementation group C (FA-C) in the subject. In some embodiments, the target gene (e.g., target FANCC gene) comprise an editing target sequence that contains the pathogenic mutation. In some embodiments, administration of the prime editing composition results in incorporation of one or more intended nucleotide edits in the target gene (e.g., target FANCC gene) that corrects the pathogenic mutation in the editing target sequence (or a double stranded region comprising the editing target sequence and the complementary sequence to the editing target sequence on a target strand) of the target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the FANCC gene in a subject, e.g., a human subject, suffering from, having, susceptible to, or at risk for Fanconi anemia of complementation group C (FA-C). Identifying a subject in need of such treatment can be in the judgment of a subject or a health care professional and can be subjective (e.g. opinion) or objective (e.g. measurable by a test or diagnostic method). In some embodiments, the subject has Fanconi anemia of complementation group C (FA-C). In some embodiments, the subject has a mutation in the FANCC gene.

In some embodiments, the subject has been diagnosed with Fanconi anemia by sequencing of a FANCC gene in the subject. In some embodiments, the subject comprises at least a copy of FANCC gene that comprises one or more mutations compared to a wild type FANCC gene. In some embodiments, the subject comprises at least a copy of FANCC gene that comprises a mutation in a coding region of the FANCC gene. In some embodiments, the subject comprises at least a copy of FANCC gene that comprises a mutation in exon 1 as compared to a wild type FANCC gene. In some embodiments, the subject comprises at least a copy of FANCC gene that comprises a c.67del (322delG) mutation as compared to a wild type FANCC gene. In some embodiments, the subject comprises at two copies of FANCC gene that comprises a c.67del (322delG) mutation as compared to a wild type FANCC gene.

In some embodiments, the subject comprises at least a copy of FANCC gene that comprises a mutation in an intron as compared to a wild type FANCC gene. In some embodiments, the subject comprises at least a copy of FANCC gene that comprises a mutation in intron 4 as compared to a wild type FANCC gene. In some embodiments, the subject comprises at least a copy of a target FANCC gene that comprises a c.456+4A->T (IVS4+4A>T) and c.67del (322delG) mutation relative to the wild-type FANCC gene. In some embodiments, the subject comprises two copies of a target FANCC gene that comprises a c.456+4A->T (IVS4+4A>T) and c.67del (322delG) mutation relative to the wild-type FANCC gene.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

In some embodiments, a population of patients each having one or more mutations in the FANCC gene may be treated with a prime editing composition (e.g., a PEgRNA, a prime editor, and optionally an ngRNA as described herein) disclosed herein.

In some embodiments, a patient with multiple mutations in the FANCC gene can be treated with a prime editing composition (e.g., a PEgRNAs, a prime editor, and optionally an ngRNA as described herein). For example, in some embodiments, a subject may comprise two copies of the gene, each comprising one or more different mutations. In some embodiments, a patient with one or more different mutations in the target gene can be treated with a prime editing composition comprising a PEgRNAs, a prime editor, and optionally an ngRNA. In some embodiments, the editing template may comprise one or more synonymous mutations relative to the wild-type FANCC gene. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer). Accordingly, one or more synonymous mutations may include a PAM silencing edit.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection or infusion or transfusion, transplantation (e.g., allogeneic hematopoietic stem cell transplantation (HSCT) using cells that have been contacted with a prime editing complex as described herein) to a subject. In some embodiments, the compositions described herein are administered to a subject by injection, by means of a catheter, by means of a suppository, or by means of an implant. In some embodiments, the compositions described herein are administered by direct injection.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are HSPCs. In some embodiments, the edited cells are induced pluripotent stem cells. In some embodiments, the edited cells are primary human cells. In some embodiments, the edited cells are human progenitor cells. In some embodiments, the edited cells are human stem cells. In some embodiments, the edited cells are human HSPCs. In some embodiments, the edited cells are human CD34+ HSPCs. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in FANCC gene, or diagnostic measurement associated with Fanconi anemia (e.g., Cytometric flow analysis) in a subject suffering from Fanconi anemia symptoms and has been administered an effective amount of a prime editing composition described herein. The level of the diagnostic marker determined in the method can be compared to known levels of the marker in either healthy normal controls or in other afflicted subjects to establish the subject's disease status.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target FANCC gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g. using pseudo-U or 5-Methyl-C.

Methods of non-viral delivery of nucleic acids can include lipofection, electroporation, nucleofection, microinjection, biolistics, virosomes, liposomes, immunoliposomes, polycation or lipid:nucleic acid conjugates, nanoparticles, cell penetrating peptides and associated conjugated molecules and chemistry, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered after delivery (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and W2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiment, the polynucleotides are a DNA polynucleotide. In some embodiment, the polynucleotides are an RNA polynucleotide, e.g., an mRNA polynucleotide.

In some embodiments, the AAV vector is selected for tropism to a particular cell, tissue, organism. In some embodiments, the AAV vector is pseudotyped, e.g., AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g. a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. In some embodiments, intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins. In some embodiments, the in vivo use of dual AAV vectors results in the expression of full-length full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. In some embodiments, insertion of an intein disrupts a Cas activity. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c. a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Cas protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gRNA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapedia, referred to as penetratin, which comprises the amino acid sequence RQIKIWFQNRRMKWKK (SEQ ID. NO: 3806). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 3823), and octa-arginine (SEQ ID NO: 3824). The nona-arginine (R9) sequence (SEQ ID NO: 3823) can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a HSPC, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 59 below.

In some embodiments, components of a prime editing composition form a complex prior to delivery to a target cell. For example, a prime editor fusion protein, a PEgRNA, and/or a ngRNA can for a complex prior to delivery to the target cell. In some embodiments, a prime editing polypeptide (e.g. a prime editor fusion protein) and a guide polynucleotide (e.g. a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 59 below.

TABLE 59
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropy 1)-N,N-dimethy 1-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy 1)-2,4,6-trimethylpyridinium | 20c | Cationic |
| 2,3-Dioleyloxy-N-P(spenninecarboxamido-ethy 1J-N,Ndimethyl- | DOSPA | Cationic |
| 1-propanatninium trifluoroacetate | | |
| 1,2-Dioley 1-3-trimethylamtnonium-propane | DOPA | Cationic |
| N-(2-Hydroxyethyl)-N,N-dimethy 1-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′, N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy 1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy 1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley 1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table

TABLE 60
Exemplary lipids for nanoparticle formulation or gene transfer

| Polymer | Abbreviation |
|---|---|
| Poly (ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly (L-lysine) | PLL |
| Histidine modified PLL | |
| Poly (N-vinylpyrrolidone) | PVP |
| Poly (propylenimine) | PPI |
| Poly (amidoamine) | PAMAM |
| Poly (amidoethylenimine) | SS PAEI |
| Triethylenetetramine | TETA |
| Poly (β-aminoester) | |
| Poly(4-hydroxy-L-proline ester) | PHP |
| Poly (allylamine) | |
| Poly(a-[4-aminobutyl]-L-glycolic acid) | PAGA |
| Poly(D,L-lactic-co-glycolic acid) | PLGA |
| Poly (N-ethyl-4-vinylpyridinium bromide) | |
| Poly (phosphazene)s | PPZ |
| Poly (phosphoester)s | PPE |
| Poly (phosphoramidate)s | PPA |
| Poly (N-2-hydroxypropylmethacrylamide) | pHPMA |
| Poly (2-(dimethylamino)ethyl methacrylate) | pDMAEMA |
| Poly (2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 6l below.

TABLE 61
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very Transient | NO | DNA |
| | Herpes Simplex | YES | Stable | NO | DNA |
| | Virus | | | | |
| Non-Viral | Cationic | YES | Transient | Depends on | Nucleic acids |
| | | | | what is | and Proteins |
| | | | | delivered | |
| | Polymeric | YES | Transient | NO | Nucleic Acids |
| | Nanoparticles | | | | |
| Biological | Attenuated Bacteria | YES | Transient | NO | Nucleic Acids |
| Non-Viral | Engineered | YES | Transient | NO | Nucleic Acids |
| | Bacteriophages | | | | |
| Delivery | Mammalian Virus- | YES | Transient | NO | Nucleic Acids |
| | like Particles | | | | |
| Vehicles | Biological | YES | Transient | NO | Nucleic Acids |
| | liposomes: | | | | |
| | Erythrocyte Ghosts | | | | |
| | and Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g. two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g. two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

Exemplary nucleotide sequence of wild type FANCC is provided in SEQ ID NO: 3809. Exemplary coding sequence for FANCC protein is provided in SEQ ID NO: 3810. Exemplary FANCC Coding Sequence (SEQ ID NO: 3810)

ATGGCTCAAGATTCAGTAGATCTTTCTTGTGATTATCAGTTTTGGATGC
AGAAGCTTTCTGTATGGGATCAGGCTTCCACTTTGGAAACCCAGCAAGA
CACCTGTCTTCACGTGGCTCAGTTCCAGGAGTTCCTAAGGAAGATGTAT
GAAGCCTTGAAAGAGATGGATTCTAATACAGTCATTGAAAGATTCCCCA
CAATTGGTCAACTGTTGGCAAAAGCTTGTTGGAATCCTTTTATTTTAGC
ATATGATGAAAGCCAAAAAATTCTAATATGGTGCTTATGTTGTCTAATT
AACAAAGAACCACAGAATTCTGGACAATCAAAACTTAACTCCTGGATAC
AGGGTGTATTATCTCATATACTTTCAGCACTCAGATTTGATAAAGAAGT
TGCTCTTTTCACTCAAGGTCTTGGGTATGCACCTATAGATTACTATCCT
GGTTTGCTTAAAAATATGGTTTTATCATTAGCGTCTGAACTCAGAGAGA
ATCATCTTAATGGATTTAACACTCAAAGGCGAATGGCTCCCGAGCGAGT
GGCGTCCCTGTCACGAGTTTGTGTCCCACTTATTACCCTGACAGATGTT
GACCCCCTGGTGGAGGCTCTCCTCATCTGTCATGGACGTGAACCTCAGG
AAATCCTCCAGCCAGAGTTCTTTGAGGCTGTAAACGAGGCCATTTTGCT
GAAGAAGATTTCTCTCCCCATGTCAGCTGTAGTCTGCCTCTGGCTTCGG
CACCTTCCCAGCCTTGAAAAAGCAATGCTGCATCTTTTTGAAAAGCTAA
TCTCCAGTGAGAGAAATTGTCTGAGAAGGATCGAATGCTTTATAAAAGA
TTCATCGCTGCCTCAAGCAGCCTGCCACCCTGCCATATTCCGGGTTGTT
GATGAGATGTTCAGGTGTGCACTCCTGGAAACCGATGGGGCCCTGGAAA
TCATAGCCACTATTCAGGTGTTTACGCAGTGCTTTGTAGAAGCTCTGGA
GAAAGCAAGCAAGCAGCTGCGGTTTGCACTCAAGACCTACTTTCCTTAC
ACTTCTCCATCTCTTGCCATGGTGCTGCTGCAAGACCCTCAAGATATCC
CTCGGGGACACTGGCTCCAGACACTGAAGCATATTTCTGAACTGCTCAG
AGAAGCAGTTGAAGACCAGACTCATGGGTCCTGCGGAGGTCCCTTTGAG
AGCTGGTTCCTGTTCATTCACTTCGGAGGATGGGCTGAGATGGTGGCAG
AGCAATTACTGATGTCGGCAGCCGAACCCCCCACGGCCCTGCTGTGGCT
CTTGGCCTTCTACTACGGCCCCCGTGATGGGAGGCAGCAGAGAGCACAG
ACTATGGTCCAGGTGAAGGCCGTGCTGGGCCACCTCCTGGCAATGTCCA
GAAGCAGCAGCCTCTCAGCCCAGGACCTGCAGACGGTAGCAGGACAGGG
CACAGACACAGACCTCAGAGCTCCTGCACAACAGCTGATCAGGCACCTT
CTCCTCAACTTCCTGCTCTGGGCTCCTGGAGGCCACACGATCGCCTGGG
ATGTCATCACCCTGATGGCTCACACTGCTGAGATAACTCACGAGATCAT
TGGCTTTCTTGACCAGACCTTGTACAGATGGAATCGTCTTGGCATTGAA
AGCCCTAGATCAGAAAAACTGGCCCGAGAGCTCCTTAAAGAGCTGCGAA
CTCAAGTCTAG

Exemplary Wild type FANCC nucleotide sequence (SEQ ID NO: 3809)

>NG_011707.1 Homo sapiens FA complementation group C (FANCC),
RefSeqGene (LRG_497) on chromosome 9
CTGCTTTCTCTCAACTCTAAAATACTTAGATCTGTGTCTTTCACACAGCGTATAGGCTTTGTGCTTTATG
CTTTGTAGTGATTGGGTTGAGAACATGCTTCTGCTAAATACAGCATGTTGATATTTGAAAACATGGCAGA
AGCTGGAAAGTCACTCTCACCTTCCCTCCTCTCCTTTCTCCTGATGCGGGTCATAAAACCTTGGAATGAT
TTTCTGAACTTCCCCTGAAGCAGGTCCTAAGACCCTCATGTAAGAAGTGCTGCCTATATCCACAGTACCT
ATCTCCACAGGAAAGGTACTTTCTTACCTCTGAAGACTGACGGGTTACAGAGAAAAATCTGAACAAATAA
GCCTTGCTAAATTCCCCCCAAGTTTATTCCCATCTTTGTTCAACTGTATCTCCCCACAACTGTCCACTCT
TCATGAAACCTGGCATAGAAACACACAAGTTTAACAGCTTCTCTGGGTCTTCCTTTCCTTAGGAAGGCTC
CTATGTCATGGGAAACTTACATGCAATGTCTATGCTTTGCTCCTGTTAATCTTTTATTATGGAGGCCTCA
GCCATGAACCTAGGATGGGTACAGAAAAGCCATTTTTCATTCCCTATAGTAATCTCGCAATAGGTATCTA
TTGATTAATTTATCAAATAGGCTCAGCCAAAGTGCGAATGCCCAGTGACACAATGAAGAGCTGCCACCTC
CAGGTTCTTAGTCAGGGCTTGCTTTGCAGTGAGGGAGAGAAACTCACTGAAGTCAGCATAGGTAAAGGGA
TGACTGTTGCAAGGATATGGGGCAATCCTGTGGACAACTAAGAACAGAAAAAATATATAGTCAGTTAACC
TTAAAATTAGGCTTAAAAATAAAAAATAAGGAATGGAAGCTCCCCTCTCTCTCACAGAATAAATAACAGC
CTTTCAACGTCATTTTGCTTTTCTCTGCCAGCCAACTTTCTTTGCTACTTACATATTTTTCTTCCCCAAG
TAACTCCAGTTGGCATGAGGGCTTTAGCTCCTGTTGTATAGAGATGTTACAGTCTCTCAGTGTTGCAATT
CCAAATTCCCCAGCAATAGTTTGATTGGTTCAGCTAGGGTCAGGCTACCATCTCTGGTTCAATTAGCTGT
GGCTAGTAATGGAGGCTCTAGTACAAACATGTCCTCCTAGGTGCATTCTTTCAGCAGAGCCTAGGGAGTG
AGCATATCGGGGGCAGGGTAGAAGTGGCAGCTTCCATGCAGCATTTATAATACATTTCCCTTTTTCCATG
AATTTCCATCTCCTGCAGCCATCCCTTTCTTGGCCTTTTCCCCTTCACGGTGGCAGATCAAAGCCCTTCA
CTATTATCTGCTCCGTTTCTTAAAGTGTTTCTGGCACAAAAAATATTCTCATTGTTCTCTTCTCTAGTTC
AACTTACATTTCCTCAGTTGGAATGTTTTAATGGGAAAGTAATGTGGTGATTAGAGTGCAGAAGCCTCTG
GGATCACCTCACCCTATTTTCAAGTCACCAGCCTGGGCCAGGAGCAAGAACATTATAAGTAAGAGGCCAC
TGTAAATGGAGTTGAAGCCAGGTTCCACCCTCCACTAGAAGAGATGGCTCTGTGCATGAGTTTCTGTATC
TGTGAAAGTGGAATATTACCACTGTGGATTACATAAGATCACTGTGAGAATCAAAGGGAATAACCCAGGC
CAAGGAGTTAGCCCAGTGCCTGGAACTTCAACACGCATTATTCACAACTGAGGTACAAGAGGCAAATATC
AAAGCCATGGACAAGATAGTGTTAAGGGCGTGATGGCATGTGCCTGTAATCCCAGCTACTCATGAGGCTG
AAGCATGAGAATTGCTTGAACCGGTGAGGCAAAGGTTGCAGTGAGCTGAGCTGAGATCATGCTACTGCAC
TCCAGCCTGGGCGACTGAGTGAGACTCTGTCTCAAAAAAAAAAAAAAAAAAAAGAAAAGATAATGTTAAA
TGTTGGGGTGCCAAATAAGGACTGGAAACCAGGTGGGTGGTGTCTGAACTAATTCTGGAAGTAGGAGTCT
TGGTACAATGGTTTCCACAACGTGGATTCTAAGACAGTTGATTCTATTTGTTGAACTTTGAAGGAAGGTT
TGATAATGTCAGGAACTTTGCAGCACTCAGCCAATTGACTTGTTTTCCTCTAGAATGTAAAGATTTAATG
TAAACGATGTAATAATTTTCACCTAAATAGGCCATTTTTCATTTTGCGACATTATTTGTGATTGGGTTCT
ATACTACAACCAAAACTGTGTTCCAGAGTAACACACACCTGGGCCCGGTGGCCCACGCCTGTAATTCCAG
CATTTTGGTGCCACTGAGGTGGTTGGATTGCTTAAGCGCAGAGGTCGAGGCTGCAGTGAGCTGTGTTTGC
ACCACTGCACTTCAGCCTGGGTGAGAGTGAGATGCAGTCTCAAAACAAACAAATCAAAACCACACACACA
CACACACACACTCCCACACACAAACAAAAATTGGATCACCCATAATCAATTTTATGATTCAATTAAAAAG
AGGGTTTGTATTGAAATATCTGTTTCTCTGGAAACTTTCTGGGCATTTTACAAAGTGAACATCTCACTAT
TGCTTAAAATATTATTAAGATAGTTCCATGTATATGAATGCCAGGCAAAATGAATCCCTATAAATATAAG
TTTGGATCTTTATGCTATATATGCTTCCTATTTAGGTCTAATTTAGGCCTAAACATGGTGTCATTTCATA
TACTGCAAAGTTGGATGAGCCTAGCAATTCTTTATTAACCACTGAACTAAAAAGACAGAAAACTGGGATA
TGGCAAGACATCAATCATTAACAAACACTAAAAATGATTTTTCAGCGAATATGTAAAAGGATGTGGGTAC
CAAAATAGATAATTTCTCTCAAGAAATACATACTTTAATTGCTTTCTTTGCTGATAAATGCAGAACTTAA
ACGTCAACTCACTCTCGATTTGTTACCACAGAGGTGACTTTCTCATGGTCAAGGTGCCACGATGAGTGCC
CCTCAGGCATGTATTTCCCTACCCAGGACTTGCTTTTGATTTATCCATGTCACACTATGCACACATCAAA
AACACATCGACCTCTCTGCTGTAAAACAGAACGAAATCATGTCCTTTGCAGCAACATGGATGGAGCTGGA
GGACATTATCCTAAGTGAACAAACTCAGAAACAGAAAATCAAATATTGCATGTTCTCATAAGCGGGAGCT
AAACAATAGGTATACATGTACATAAGAATGGGAATAACAAGACACTGGGGACTCCAAAAAGGGAGAGGGT
GGGAAGGGGGTGAGGGCTGAAAAATTAGCTATTGGGTACAATGTTCAATGTTTGGGGGACAGGTACACCA
GAAGCCCAATCCCCACCATTACACAATATACCTATGTAACAAGCAAGCACATGTACCCCCGAATCTAAAA
TTAAAAAACAATCAAAAAGCACCGACTTCCTTAACTGGCATGGAATAGTTTCGAGAATGGATGTAAACAC
CGAATTCCTTAATGTAGTTGCAGATTCTGCCAGTTTTGTCTCAACCTCTGGATCGGTTCCTTTTTTTAAC
TACCTTTAATTCTTAAACAGATTGTTGCTATTGCTATGCTATATACTTATTGTATCACTTATAAATGTAT
AACCTCTGCAAATTAGTCTCTGAATTGAAAGTATTCATTGGTAAAACGGGTATTTCTTCACCATAGGAAT
AGTTGGACATGTTTAAATACTTGAGAGCTATTTTTATTACAAGCACACACAGCTTTGTGGTGTTTTTTGT
TGAGACAGAGTCTCGCTCTGTTGCCCAGGCTGGAGTGCAGTAGTGCGATCTTGGCTTACTGCAACCTTCG
CCTCCTGGGTTCAAGCAATTCTCCTGCCTCAGCCTCCTAAGTAGCCGGGAGTACACACTAATTTTTGTAT
TTTTAGTAGAGACGGGTTTTTGCCATGTTGGCCAGGCTGGTCTCAAACTCTTGACCTCAGGTGATCCACC
CGCCTCAGCTTCCCAAAGTGCTGGGATTAGAGGTGTGAGCCACCGCGCCCGGCCTGTAGTGTGTTTTTGA
ACGTATATGTGAAGTTGTAGCTTTTTTTTTTTTTTTTTTGAGACGGAGTCTCCCTCTGTCGCCAGGCTGG
AGTGCAGTGGCGCGATCTCGGCTCACTGCAACCTCTGACTCCCTGGTTCAAGCTATTCTGCCTCAGCCTC
CAGAGTAGCTGAGATTACAGGCGCCCGCCACCGCGCCCGGTTAAACGTTTTGTATTTTTAGTAGAGACGG
CGTTTCACCATGTTGGCCAGAATGGTCTCGATCTTCTGACCTCGTGATCCGCCCGCCTTGGCCTTCCAAA
GTGCTGGGATTACAGGCCTGAGACACAGCGCCCGACCTAAGCTGTAGTTTTATGATTATGATTTATGGTT
GTGAGGCATTGATTTAAGACATTTTAAAGCTTAAGTCGTCAGATCCTAGAAGAAAAAAAAATTCACCAAG
ACAAGCTAATGCCATTGTCATAAATGGGAGTTTGGAGAAAATTTTATTAATATTTGTTTGGGTGGTTTAT
GTACAGCGTTGAACTGATTTGAACCCCGACCAAACACTGTATGGAACTAATGTACGGTAGGGGAAATCAT
TAACTGAAGATGCCTTTTTTGTTGCTATTGTTTAGAACGCAGTTAGAAAATCTTATTTTAGAGGTTTTAC
CCCGTTGACAAAGCCTCAACTTTTCTTCAAAAGAACTAATTTCCATTTTACCTCCCCTACATTTTGAACA
GAATATGGAAGAAGCCAGCGCCCCTTCTCCGCGGGCGCTGCTGCAACCCTGCTCCCGCCGGGGCTTCCGC
GCGAAACCGGGAACACTGGAGCAGTACCGACTCCGCTCGCCTAGAACCCGCCCCAGCCGGGCCCACTTCC
GCCAGAGCCCTGGCCGCGCCCGCCCCGCCCCACGGACCCGAGATTCGTCGGAATTTTCCCGCGACGCCGC
GGGAGCGCGCGGGGCCGCTAGCCAGGCCCCAGAATGCACTGCTGACACGTGTGCGCGCGCGCGGCTCCAC
TGCCGGGCGACCGCGGGAAAATTCCAAAAAAACTCAAAAAGCCAATACGAGGCAAAGCCAAATTTTCAAG
CCACAGATCCCGGGCGGTGGCTTCCTTTCCGCCACTGCCCAAACTGCTGAAGCAGCTCCCGCGAGGACCA
CCCGGTAAGCGGCAGGCCGCAGAACGCAGCGAGGGCGGAGGCTTCCCTCGGGGGTCTGGCCGACACGTCA
GCGGCGAGAGCGGGAGGAGGAGGCGAGGGGCAAGAGGGAAGGCTCGCGAGAGCGGGTGGGCCAGGCGCGC
CTTGCGGTGGGCGAGAGGGGTTCCCGCCGGCGCAGGGCTGTGGCTAAGGCTGAGGCGGGAGGCGGGAGGC
GGGAGGCGGAAGGTGGCGGGCGGTGCCAGCCCGCGGGGGGGGGCCAGGGCGGGGAAAGCGGGGGCGGGAG
CCGGCGCTGGGGTCGCGCGGAAGGGAGCCCCCGGAGAGGCGGGAGCCGGGTGTTGGCGTTTTGGTTCTTT
TTGTTCATTGAGCGCAGGCAGCTATGTCTTCTTCAAAGGAGAGGAGCAAAGGTGAATTTCTCTGCCTCGG
ACTCGTGGGAGCCGTGAAAAGCTCGCCCCTTTGAGCCGGTCGCCCTCTACAGCTGGCCTCGTCCTCCCCG
TCCCTCACACCCCTGCCCCCAAACCCCATTCGCCGGGGCGCCAGGAGGGGCGAGGTCCAATTAGAGGAGC
CCGGGGCGCGGCTGTTACCCCTCCCGCCCTCCGCCTTTTATTCCCGGAGGGAGGCGGGTTCGCAGAGGCC
AGTGTCTTCCCTCCATTCTTTTTGTGGACGCCAGGCCATATCTTCAAAGCCCGTGAGAGATTTTTCAGGT
GCTGTTTGTTGGTTTGCTCTTTTTAATTTTTTAGGTGGGTGCGTTTACAACTACGAACCCTCTATGATCT
TATGTGTCCAGCGCACCGAATGGGGGAAAAGACTTGGAGTTTTATAACATGGGAAATTGGGTACCAGAAT
CTTAAGGGAAGAGACCAAGGAGTTTTCTAGCAAAGTCCTGTATGTGCCTGGACTTTTGTTCTAATCTGAA
ACTTGGAAAACTCCCGGGCATCTGATTTTATTCTAACTTGTGCCCCATTTAATGCATCTAAACTGTGATG
TGATTTGACTTAGCATTTATCAAATGAGCAGAGCACTTTTGGGCCACCCAACTTCAGCTCTTTGTGGCTG
TCACTCACTGTCAGTGATATGCCCTCAGACAGATCGTTTTCTCCTTACTCCCCTGAAAGGTTGTTACAAA
CAGTAGAATTCATACCATGTGTTGAGATCATTCATTACCTAAGCTTAGATGGAAAACATGTTCATTGCGA
CTTTATACATTAAACTTGAATTGATGTTTCAATGTTATGTGCTGTACCAGAGTCACCTGGAAATAAGCAC
TCATATACATTATTCATATACAAGAAAGGCTGCATCGAAAAAGAATTTGTTTTCCTAGATATATCAGAGA
TCTTTTTCACCAACCCACTACTCCCAGTAATAGAGCCTGCTTCTTGAGAACTGGACTAATTATACTTTGT
ATATTATACATTCGAATTTATTTTTGCTTTATTCGGTCATATTTTCTCTTGATTTCTCCAAAAACGTTTA
CCAAGGCATCAGAAAAGAAAAGCAGGCTCTATATTGGTATTTTTTGTTTTGTCCTCTAATAAAAAGCATT
AACTTGATTGATTGGCTGGTTGTGGAAGATGTGTTGCTGCATGTATGTGCAACTGTGCATGTACATTTTA
AAACAAATGGTACTTTTAACCAGTTCCATATTTCAGCAGGTAGTTCCAATAACAATTATACAAATTTCGT
GTTTATGAGAACCAGCTACAGGGAGGCCAGCCTGGCCAGTAGAAAGTTGGATTGATTATCTGGGCCATGG
AGAGAGCCAGAGGAAACTCACATGAGTGGTAGTTCCAAGTTGGTAGTGTTAGGGCCTCAAATCGGGCTGG
AAATGAGCACCAAATGATTTTTAAAAGTTTTATTTTAGGCTGGGATATGTGCATTCTCTGTCTTGTTTGT
GGATCTTGAGTGATTGACTTAATGTGAAAAAATACATACAGGAGAAGCACCTTGGAAGTTTTGCAATGTT
TTAATAAAAATGCAGAAGTGGAGAAGGAAAGGCAATTCAGATAACAGGCAGAGCATGGTGGCTTATGCCT
GTAATCCCAGCACTTTGGGAAGCGGAGGCGGAAGGATCACTTGATCCCAGGAGTTCGAGTCCAGCGTGGG
CAACAGAGAGAGCCCTGTCTCTACAAAAAGTTAAGAAATTAGCCGGCCATAGTGATGCGGGGCTGTGGTC
CCAGTTACTCGGGAGGCTGAGGTGGAGGATCACCTGAGTTCAGGAGGTCGAGGCCACAGTGAACTGTGAT
TGCGCCACTGCACTCCACACTGGGCAGCAGACTGAGACCGTGTCTGGAAAAATAAAATTGGTAACAATTC
AGATTGGCCACCAGAGTTGGACTGGGGGCTAGCAGGCAATGGTTTTACCTCCATTTTGAGATTGGCATCT
TCTAACTCAAGTTTAATACGGGACTGAGGGTAAGAGACCTGCATGCCTTTTATGTCAGCATTACATTATC
TTGATTATGGCAGCTTTATAGTAAGTTTTGAAATTAAGTAGTGTAAGTTATCCAATGTTTTTCTTCATTT
TCAAAATTGTTTTGGCGGTTCAGGGTTCCTTACATTTCCATATAAATTTTAGTATCAGTTTGTTATTTTT
TTTCAGAAAACCGTGCTGTGATAGACTTTGTAGGAATTGCATTTAATTTTGCATATCTATTTGGGGGAGA
ATTGCCATCTCATCATGAACATGGAATCTCTTTCCATTTGTTGAGCGCTTTAATTTCTCTTAGCAGTATT
TAGTAATTTTCAGTGTACAAATTTTGCCCTTTTGTAAATTAATTTTGAAGTTTTATTTTTGATGGTATTG
TGAATTGAATTATTTTTAAAATTTCATTAAAATTTTTTTTATCGATAAAGTGAAAGCAAGTTTTTTGTTT
TTTTTTTGAGACAGAGTCTCGCTCTGTTTGCCCAGACTGGAGTGCAGTGGCACGATCTCGGCTCACTGCA
ATCTCCGCCTCCCGGGTTCAAGCGATTCTTCTGCCTCAGCCTCCCGAGTAGCTGGGACTACAGGCACCCT
TCACCACGCCCGGCTAATTTTTGTATTTTTAGTAGAGACGGGGTTTCACCATATTGGTCAGGCTGGTCTC
AAACTCCTGACCTCGTGACCCGCCTGCCTCGGCCTCCCAAAGTGCTGGGATTACAGGCGTGAGTCACTGC
GCCCGGCCGAAAGCAAGTTTATTAAGAAAGCAAAGGAATAAAGAATGGCTACTCCACAGGTAGAGCAGCC
GAATTATTTTTAAAATTTCATTATGGATTGTTGTCAGTATATAAACATACACTTGATTTCACTTATTAAT
CTTATATCTTGCAACCTTCCTTGTTGAACTTGATGATTACTTCCAGTTTTTTGTGAATTACACAGGATTT
TCTACATACGGCATCATGCCAATATAGTTTTACTTTTCTTTCCATGTGGATGCCTTTAGTTTCTTTTTTG
CCTGATTAATTTTTTTCTTACCTGGTTCGACCCTCCAGCACTTTGCTGAATAGACATGGAGTGTATATTA
TTGACTTATTTCTCATCTTAGGGGGAAAACATTTAATCTTTTACCATTAGATACGACGTTAGCTGTAAGT
TTTTCATAAATGCACTTTATCAGGTTGAGAGATTTTCCTTCTGTTACTAGATTGTTGAGAATTCTTATGA
ACAAATGTTAGAAATGTTGCTTTTCTGCATCTATTGAGATGACTGTGGTTTTGTTCCTTTATCAATATGA
TTTATTAACTGATTTGTGTATTTATACTAACCTTGCATTCTAAATCCCATTTAGTCATGTTATATAATCC
TTTTTACATATTGCTGGATTTGGCTTAGTAATATTTTGTTTAGGATTTTTGTATTATGTTCATGGTAATA
TTAGTAATATTTTGTCTTGTGATGTCTGTCTGGCTTTGGTATTACCGTAATAGTGGCATCTTAGAATGAG
TTGGGAAGTGTTCCCTCCTATTTTTTCAGAGTTTGTGAAGGATTGGTATTTTTTTTTCTTCAAATATTTG
ATAGAATTCACTCACAAAGTCATCAGTTCGTGGATATTTCTTTATAGGGAAATTTGAAATTACAAATTCA
GGTTCTCCAGCGCACGCATGCGTGTGACTTCTCCGGTGAAGGTTGTCGTGCGCGCACCTGGCCGTGACTC
CTCCAGAGCGAGGTTGTGCACACATGCGCGCGCCTTCTCCATCGCCAGCTTCCACATACTTTCTCCAGCA
CTGGTTCCCAGGCACACACGTGACGCTTCTCCAGGGCGGAGCTCCCATAGTGTGTGCGGCTTTCTAGCGC
TCACTCTTGGTGGGTGTGCTTTGCCAGCGCTAGCTCCCTGGGGTGTGCCGCTTCCCCAACGCTGGCTCCC
GTGCTTGGGTGCCGCCCCAGCACCTGTTCCCAGGCGTGTGCACGTCTCCAGCGCTAGGTCCTTGGCATAT
GTGCTTCTGCAGCGCCGGCTTCTGTGGTGTGTATCTTCCCCAGTGCAGGCTCTTGTAGTGCGCTGTGATC
AGCTGCACCCAATGGTCAACTGTGTACCTCAGCATCCCCCTCAGGCGATTTTGTAACTGAGCCTGTGGTG
GGACACCTCCCTGTGAACACTTTTCCTGGCGCCCTAGAGGATGGGTTTCTGGCAAGTTACCAAAGGTGGT
TTTCCAGCAAGTTGTACTGGTACAGCACCCACAGAGACATCTCTCGTATTCAGTGAGCCATGGCTGTGCT
CCCTCCAAAAAGTTCTTGATCCCATTTCTGGGAAGAAGTGGGTATTTTTCCTTAGGGCACCCACCTCAGC
CCTAGGGGCAGTGGCTCCTCCTTATATCTCCTATTCCTGTGTTTTGTAGAATTCTCTGTACTTCTTACTA
GCCAGCACCTTGTTACTCCAGTCCTGTTACAGTGAATAGTTCTTGACTGGGCATGGTGACTTATGCCTGT
AATCCCAGCACTTTGCGAGGCCGCAGGGCTTGGGAGAATTACTTGAGACTAGGAGTTTGAGACAAGCCTG
GGCAACATAGCAAAACCCCTGTTCTACAGAAAACACGAAATTATCTGGGAGTGGTGTGTGTCCCACCTAC
TACGGAGGCTGATGTGGGAGGATCGTTGAGCCCAGGAGGTTGAGGCTGCTTGGAGCCATGATTATACCAT
TGCATTCCAGCCTGGGCAACAGAGTGAGACCCTCTCTCTAAAAAAGAAAATGACAACAACAACAGTAATT
ATTTATAGTTGACTTTCCTTATTCAAATTATTATGTGGTTTCTTTTTACTGACTGGACCCAGACTGATTT
ATCACCTTTGCTCTGAACTTCCTCAAGAAATCATGAGTGGTGAGAGTATTTAGTCTGTTCATTTTCTTGA
ATTGTGAAAGTTAATCCTAAGGTTTCTCCCACTTCCACATGACGAAAGACACGTTTGTGTGAGAGTATGT
AGGCTTTTATGAAGTGATGAAACTGCCTTTCCATGCCTTTGCAAATTAGAAACAGTATAGATTATCATGG
AAAGTGGGGTCAGAGATTAGGAGGGGAGGAGAAGAGGACAGGATTAGAACTGTGGTCGACCTTATGGCAT
CCAGTGGGCAGGGTTCCCAGATGACCTGACTTTAGATGTGACTGTACTTCTCATTATCTTTCACCCCTGT
TTTTCATTTTTCAGATTTCTGGGACTTGTGTTTTCATTTTCCTCTAGAGCCCCAATGATGCTTTTGTAAT
TCAGAAACATTTATGTGTGATATAAACATTTTATATACATTTAGCTATTTTAGTGGAATCTGAGATCTGT
TACTCTGGTGACTACCCATAAGACAGTTTGAGGCTGCGGTTTTTCACACACACACACACACACACACACA
CACACACACAGATTCAGAGGAGACTATTTAAACAATATGAATTTGGCTGGGTGCAGTGCTGTGCACCTGT
AGTCCCAACTATTTGAGAGGCTGAGGCTGGAAGATTGTGAGTTCAAGTCTCCTGGGCAAAATTGTGAGAC
CCTATCTCCAGGGAAAAAAATAAATAAATAAAAGGCTATGACTTTATGTCTGTGATTCCTTCTTTACCTG
GTTCAGTTCAAAGGTATGGAATTGCCAGACACTTTTGCTTCTTTTATAAGATTTTGGAGGTGCCATTTAC
CTTAGTGACTTCCAACACCCTCCTATGGCTGGTTCCTTTTTGTTGTCATTTTGTTTTTGTTGGTTGTTAC
TGTTTACTTAAAGCTATAACATTATAAGGCATTGTACTTAGCCTAATCCTATTTTTGTAATTATGCTTGT
ACATTCAAGTGCTGTACTCAATAGATTTCCTTATTAACTCCTGATTTTTTTTTTTTTTTTTTTTTTGAGA
CGGAATCTCACTCTGTCTCCCGGGCTGGAGTGCAGTGGCGCGATCTCGGCTCATTGCAAGCTCTGCCTCC
CGGGTTCACACCATTCTCCTCCTGAGTAGCTGGGACTACAGGAGCCCACCACCACGCCCAGCTAATTTTT
TGTATTTTTGTTAGAGGCGGAGTTTCACCGTGTTAGCCAGGATGGTCTTGATCTCCTGACCTCGTGATCC
GCCTGCCTTGGCCTCCCAAAGTGCTGGGATTACAGGCGTGAGCCACCGCTCCTGGCCAACGCCTGATTTA
TTGATGACCATTTGTGGTTATGCATAATGACTTTTCCTCTGTCTTTGTGCACTGCAGTTCATATTATTGC
CTTATGTGTATATGCTTGTTTCCACAACTCTTAGCCAACTTCTTCAGGGTATATTCTTTTTTATTATATA
ACTGCATTGATGTTTGTTGAGTGCCAACATCCTTTCCTCCCTTCTCTGTCATAACAGACCCTAGATTTTC
TTTTAGTAGTCACTATTCTATGGGCTCTGGGGAACACTGATACTACCTCTAGCTCCTGGGGGTGGACTTT
TATTGGTTTAAGGAGTGTGTTTTATTGATTCGTCACAGTCATTGGTTAAGGATGGGCATAAGCTCAAGCA
AGTCCAGTCTGAATACATCTGAGGTCTGTCTCTTACTTGGGATGCTGGAACAGAAGTATGTACTCTATTT
TTGGGTGGTATGGATGTGATGTGACTTCTGTAATGGCTAAAGCTGTTTTGCTATCATGAGGGAAGCAGGA
TGGAGAGACAGATCAACTGTGAAGTCTGCCCTGTTTCTGGACTTGCCAGTTTCTTGAACCAACGAATTCC
CCCCCTCCTTTTTTTTTTTTCTCTTTTGAAACAGGGTCTTGCCCTGTCAACCAGGAGTGCAGTGGCACGA
ACATAGCTCACTGCAGCCTCAAACTCCTGGGCTCCAGGAATCATGCCACCTCAGCCACCTGAGTAACTAG
GACTACAGGTATGCACCACAATGTCTGGCTAATTAATTATCATTATTTTTGTAGAGACAGAGTCATGCTA
TGTTACCTAGGCTGGTCTTGAACTCCCAGCCTCAAGCTGTCCTCCTGCCTGGGTCTCCCAAAGTGCTGTG
ATGAGAGGTGTGAGCCACCATGCCTGGCCTCCCATTATTGTTTTAGCTGGTTTTGCTTGGTGCAGCAGGG
GCATTTAATGCTTACTAAATATTTAGATCTTCCCTCACATTTCCCAGTTCACTTGCAGCTAGGTAGGACA
TTGTGATTGTTCTAGCCAATAGATTTTAAGCAGGTTATTTCCAGGTAGAAGCATTTAGTAGCAGATTCCT
GACCTAGCTATCTCTTCTTTTTGCAATGGTGATTCTGGAGGCTAGGTGTTGAGATGTCTGAGCACCTGTC
ATGCTGGATCCATGAGTGACTGTGGAGCAGAACAACACCCCCACCCCACAAACATGTGTGTCCCACATAA
CATGTTGGACATATAGTATCAGAAAAAAGTTACTACATCATAACTTAGCCTATCTTGATGAATAGAGTTG
GGTTTTCAATTACTGGAAACCAAAGGCATCCTAGGTGATATAGTATAATTTTGTAATTCTTAGTGTGTAT
TTGGTGATTTACAATGTATGCAGATTATACATTGTAATTGATTAGTAAGACACTTGTCTGAAGGAATCAG
TTATATTTAGCCCTAGCTTTCTGAATTTGGTGTACCCAAACATGACCTTGTCCAACATGGATTGTAACGA
TTACTTAGCCTATCTACCCCTACATATTAAAGATTATAATAAAACACTATATAAAATATACATTTATACA
AATAATATCGAATTGCTGCTTTTGCTTGAAGCCGTTAGCTAGGAATTTCCAATCATAGGCCGTAAGAGTG
AGTAACCGTTTATTATTGGTATAACAGCATGCATTTTATTTATGAAGTAGTATTTTCTGCAGTATTTGGC
ACCCAGAAGAGTATCTTGTGTTTCTGATACTGACTAGTACCATTAGAATTTTTAGTCATTACACTAAAAA
AGTTGGCAAAGCTAGTGAAACTTTTGCTTGATGATAAGTATTTTGATAAGTGGTGACTTTTTGAAACTAT
GCAGCCAGTTTTAGGAGTAACAGAAATATTTTTATATGAAATGGTATAATATGACTGATTTTTTCTTAAA
ATTGAGTAGTTGTAAGCAAGAAAAGTGCAATACAAATATCTTTTTCTTTTTTCGAGATGCATCTCACTCG
GTCCCCCAGGCTGGAGTGCAGTAGTGCAATCATAACTCGTTGCAGTCTTGCCCTCCTGGACTCAAGTGAT
CCTCCCACCTGAGCCTCCTGAGTAGCTGGGACTACAGGCATGAGGCAGACACTGTGCCTGCCCTTAAAAA
TATCTTGAATTTTGGTAGTGACATTCCTTTGAAGGTATTAAAAGCTAGAAATTTGTTTCATTCTATCAAA
GTTTCAGGTATCTTCATTTAGTTCTACACGTGTGTGCTGTACCCATCTAATGTTTGCCTAAAATTGTACT
GTCTTTTTGGTTTTAAAATAATTTAAGTCTAGGCCGGGCGTGGTGGCTCATGCCTGTAATCGCAGCACTT
TGGGAGGCCGAGGCGGGTGGATCACTTGAGTCCTGGTGGTCAAGGATGCAGGTCGGCTGGGCAAGGTAGC
TCCTGCCTGTAATCCCAGCACTTTGGGAGGCCAAGGCGGGTGGATCACTTGAGGTCAGGAGTTCGACTCC
AGCCTGGCCAACAGGGTGAAACCTGTCTCTAATAAAAATACAAAAAAAACTAGCCGGGTGTGGTGACATG
TGCCTGTAATCCCAGCTACTTGGGAGGTGGAAGTACAAGAATCGCTTGAACTCGGGAGGTTGCAGTGAGC
TGAGATTGGGCCATTGCACAAACAAACAAACAAAAAGCGGTGATCTGTGATTGCACCACTGCACTCCAGC
CTGGGTGACAGAGCAAGACCCTATCTCAAAAAAAAAAGAAAAAAAAATTAAGCCTAAGATGTTTAATTTA
AAAATAAGTTTTTAAAAAAGTATGTTTTTGAGGCAAAATGGTGAGAATAGGACAAGTATTTGAAAATATT
GTGGTACTTGATCAGAACCACCTTTGGTTACATTGTTTTGTGGCCAAAGTGGCTATGACTAAATGACTAC
ATGTTATTTTAAAAACATTGTTTCTAAGGTATTACTGATGTACATGAATGGATTGATATTATCATGGAGT
GGTTTGTTATAAAAGCGAGTTTGGCCCTCTTGTGCTCTGTCGCTGTGTGATGCCTTCCATTGCGATGTGA
CCCCTCAATCTTAGACTTCCCAGCCTCCAGAACTGGGAGCTGATAAATTTCTGTTATTTGTAAACTACTG
AGTCTCACATATTCTGGTATAGCAGCACAAAACAGACTAAGACAGGCAGTTGGGCAACAGCAAGTCTGAG
TTCCCTTCCAGCAGCAACTGGCAGGAGTGAAATCCTGGCTGTCTTCTTTGCAGTCAAATGCTCTACCACT
GAGCTATACCTACTGGCTATCTTCTTTGGACAGTTGCTGTTTTTCACCACAAGCCTGTCTAATTGCTTTA
CTCATTTAACTTATCTGTTGTGGACTTTGAATTTATGACCTTTGTCTAAAGTAATTTAAGTTTTGAATAA
TTCTGAAATGCATTCAGTTGTTAAACTAACACTTAGAAAAAAATGGACAGATCAGAACAAATTTGTCATA
CTTATTGGAGAAATTACTGCATTAATCAGGGTTCTCTGGTGACTTCTAAGTTTCATATCTGTAGAAAAGG
TTATTATTATTCTTGAGCAGTAAGATACTGTATACCCTTTATAAATCAGAGCTTTTTGTTCTGTTGATTT
TATACAAATACGTTGGTTTTATAAGCATGGTTTTTTTTTGTTAACTACTTCTTTTGATTTCTTAATTTAT
TTCTAACATTACTTTGGGCAATAGCCCTATTAAAAGTGCTTTGTACTTTCCAAATTATTTCCATATAGAT
TATATGGGATTCTCACAATAACCATATGTGCTAAGGTAAGATAGGCATTTTAAAAAAAGAAAAACAGGGC
TGAGCACCTGTAATCCCAGCACTTTGGGAGGCAGAGGTGGGCAGATCACTTGAGCCCGTAAGTTCAGGAC
GAGCCTGGGCAACATGGCGAAACCTTGTCTCTACAAAAAATACAAAAATTATCCAGGCGTGGTGACATGC
AACTGTAGACCCATACAGTGTGACATGCAACTGTAGGCTGAAGTGGGAGGATCGCTTGAGCCTGGGAGGT
TGAGGGTGCAGTGAGCTATGATCATACCACTGCACTCCAGCCTGGGTGACAGAGCGAAACCCTGTGTCTA
AAAACTAAATAAATAAATAAAAATTAGAAAAGAAAAATAGAATGGAGACTTTTCAAAAAAATTACTTTGG
CTCATAATTAAGGAATTCCCAAGAGATTGCCCAAAGGAGGATAGCTACTGATGAACTTCTTGGGTTGATT
GTGTGTGTACAGCTTTTAAATGTGTCTTAATTGTTATGAGATGATAACCTTTTCAATAATCAAAAGAGCC
GGGTATTTCTGGTTACCCAAACCCAACTGATTTTAGCAGAAAACATTTGCCTTAAATAATAACTTCTCTT
TGAAATGTTTAGAGAGAAGGAAACTGAGTTTAATGGCAGGGTTGTTGGCTGTTACCCAGGGAAACTAACT
CCTGTCTGGTGCTCTTTTTGTTGCACCAGTAGTTCGATGTCCAAGGGGGGCTGAAGCTTCCTATGGAAGC
ATTGTTAAGCAAGTCTTTCGCAGGCTTTCGGTTGCATCTTTCTACTGTGTAAATAGGGGCATTAGGAGAA
ATGTAATCTTTGAATACTGCCCAACCAGTTTCTGCTTGGTAATGTGCCCTGAAACCCTCAATGAATGCCA
GCAAACAAATTAGATCCACAGTGTATTTGATACGCATGTCTTTTCCCCAAACAAAATTCAAATTTCACAA
ACACAAACTGAGTCATTTTCCAAATGTTTGTATATGAATGGAAGTTTTTGTCCTGTTCTTCTGTAACCAA
TACCATAAAACTTCTGAAGGTCCTTGATGCATTTATTGTACAATGTTAATCCAAAACAAAACTTTATTCT
GACTAAACTTGTAGGTATAGAGAGATACCAAACTCATTTTTGTTGATCAGTACAACATGTTTGTACCAAC
CAGATCTAAAAAATGTGTAAAGGAGTGCCATGCTGTAAAGACCTTATTACAGTTGTAAACATCAGTGCAG
CTGTTACAAATCTAGCAAAACCTCAGGTTAAACAATTTCAAATTTACTGAAGATGACATTCCAGACCCAG
TTTTCATAACATAAACACTCTAGGAAATGCCTTTGAGATGAGTGGGATTGTCATTAATATTAATGCCACC
AAATTCAAGGCTGAGAAGTCATTTCTTTTCAAGCTTTATTTTCACTCCTAATTTTGGAATAAAATATACT
TTTAACCTGAAGTATGGCGTGTCTTTTCACAGCCTGAAAACATTTGATCCTGTTGCCTTTGAATTGTAGG
AATGCAAAAATACAGTATAAGTCTTTTATTCTTGATGGTCATTTAACTTAAATGAGTTTGTTAGAATTAA
AGTTGGGAAGAATTAACTGTTAATGGAACTCAGTTGCATTGTTCAATTAAAAAATAAAAGGATGCTAAAA
ATAGTTGCTGTTTAAGGATTGTATTAGTGTGACATTTTAGAATGCTGCATTTAGGGAAGGAATTTTATTC
TAAAGTGATTTGAAGCACATTTTTTTGTGGTCCTGTCTTTCATCTAGACAAGGTAGGACAAGCATTCAAA
ACCTGTACCAGTCTTAATCTGTTTTTTTCTTCTTAGTTTCCACTTAGTGGGACATCTGGTGAGGTATCAT
TTTGGCCATATAGTCATTTCTAAAGTTTCTTTCTGTGCTCTGATTTCCAATTAAGTCTTCTATGATGTTT
GCCAAATTTAAGAGCATGCAGGGGTCCACTGGAAGTGCTGAATAACTGGGCTTTGGCCTAATTCTTTGTG
CATGGCTGTATGTGTGGGGTGATTTTGTTTTCTTTGCTTGTTTATTGGTAATTGCATTAAATTCCATTTG
CCTTTACTGAATTTTTTTTTGTTGGACTTTTGGGGAAACCTCTAAATTGGATAATAGAGCAATTAAGTGA
CTGTTTGTAAGCGATCCTCAGTGCTCTATTAGAGAGGCAGCATTTATCAAAAGAAAACAGTTGAATTTCT
CTTGTTTAAACTTGCTTTTGAAATTCTGTTAATTTTCTAATAAGGAGAATTGAAGCCAAGCATTGTCAAG
TACCATATTGTAATCTGAAGTTGGTAACTGTGGAAGCACATAGTTGATATGGTGATACCTTTTTCAGAGG
AACTTTGGCTATTTCCATCAGCATAAAGGACACAACAGACTGCATCTATTGCCTTCACCTCTGCCTTATT
TCTTCTGTTTTCCCTTTTTTTTTTTTTTTTTTTTTTTTTTGAGATAGAGTCTTGCTCTGTTGCCCAGGCT
GCAGTACAGTGGCACGATCTTGGCTCACTGCAACCTCCGCCTCCCAGGTTCAAGGGATTCTTGTGTCTCA
GCCTCCAAAGTAGCTGGGATTACAGGCGCTCGCCACCACGCCCACCTATTTTTTTTTTTTCGTTTTTAGT
AGAGACAGGGTTTCCCCATGTTGCCCAAGCTGGTCTTGAACTCCTGGGCTCAAATGATCTCCTCCCTCAG
CCTCCCAAAGTGCTGGGATCACAGGTGTGAGCCAACACTCCTGGCCCCCTTTTTTTTAAAAAAAATTAAA
GAGGTTGTGGAGGTTTCTTTGAATTCTTTTGGTGTTTTTAAAAATGTTATATTTTGAGAAGAAAATCTGG
GCAGGTACAAAGAACCCCTCCCTTTCCTGTTTGAAAACTTTTTTGCAGTCGCCCTGGAATTTTCCTTCAC
TGTCATTCATCCTTCTCTCCTTTCTTGATATTGGGTCTTCTGTTTACTTTATTTTCTTTCTTTGTTTTCT
CCCTCCTCTTGATGTGAAGCAGATGTTCTAGTATCTTCCTAAAGCAGAATGTGTGGGAGATGAATTATTT
CTAGTCACTGCCTGTTGAATGTGAAGTTATTCTAGTCACATTTGATTGATAGTTTGTCTAGATAAGAATT
CCAGGTTGGAAATTATTTTCCTTTAGAATTGTGAGGCATTGTTTAATTTCCTTCTACCTTCCTGTGTTTT
TACAATTTCATGATGATGTGCCTGGTGTGGATCTCTTCTCATCCATTTTCTTGGGCACTTGGTCAGCCCT
TTCAATCTGGAAACTCCTGTCTTTTCGTTCTGAGAATATTTTTGGTGATTATCTCTTCCCTTCCATATTG
GAACTCATTACAAGAAAGCTAATTAAGGAAGAGTAGAGGGTTGTGTTATGGGCTGAATTGTGTCCACCCC
TCCCCCATTCATAAGTTGAAGCTGAAGCTGTAACCCCCAATGGGACTGTATTTGGAGATGGAGCCTTTAG
GAAGTCATACCGGTGGGGTCCTAATCTGGTAGGATTGGTGGCCTTAGAAGAAGAGGTAGAGAGAGAGATC
TCTCCCCACATGAGCACAAAGAAGAGGTCATGTGAGGACACAGGTGGCTGCCTACATACAAGCCAGGAAG
AGGGCTCTCAACAGGACCTAATAGGCCTTGATTGTGGACTTTACAGCCTCCAGAATTGTGAGAAAATACA
TTTCTGTTATTTAAGCCACCATGTATGTGATATTTTGTTATGGCAGCCTAAACTGACGAAGACAGGTTGA
GGTCAAGCTAGGACCTCAGCTTCATAAAGGTGAACTGCATGCGTTGCAAGAGAATATTAACATGATGCTG
GTGCGCGTGTGTTCTCCTTTTTGACTGGTCCCTTTTTGAACTTAGCTCACTCATCTGCATATCACCCTTT
GTGAGTTAGCACAAGTCATTAACCCAAGGTTCTAGATCTGTGCTGACCAATGCCATAGTCAATAGGTACG
TTTAATTATATAAATTTACATTAATTAAAATTAAATAAAATTCAAAATTCACTTCCTCAGATTCATTAGC
CATATTGCATGCGTTCAGAAGCCACATGCAGCTGGCGCCTGCAGTATTGGATGGACAGATAGAGCCAGAA
CCTTTCCATCCTTGCAGAAAGTCTGGCTGTACAACCGTATTCTAGATACTTGCCTTACACCTCTCCCTCT
CTCTTTTTTTTCTTGTCCAGTGGGTCTGAACTTTTTTAGGTTCAAGACATACTTAGGAATACTAGAATTT
GTAGTGGACATCTTCAAGGTTTTAAAACAACACTAAGAAGTAAACAGCCATTATAGTGCAAACTCTGCCA
AAGTGTCATATGCATACTGAGTCTATAACTGCGTCTGCAGTGATCATGCTATTCATTTGGCCATGCTTCT
GCAGTATGGCCCTTAGAAGAGCTGTTTTTTACCCCTACTGGGCTTTCTGATATGTTTGTAGGACAGAGAA
TTGCAGGAAGCAGGCCTTCATATCTGAGCCGTGCTGCTCTGGTTTGCTGTGCTGAATGTTGCTGTGCTCT
CCCGGAAGAGGTAGGCACAAGTGCTTTAGAAGGAACTGTCAGAACCCCACTGCTCTACTGAGCACTGGTG
AGTCTGAGGATTCACAGGCTAAAGGCAGGTCACTGAAAGCGGCCTACATTCTGGAATTTCTCGGAACAGA
TTATTTCCTGAGATGACAAAAAGTATAATTTTGGTCAGAAGGATCACTGAATTTCCTGGAATCTACCCCA
CCAGGCTGTGTCTTCATAGGTGGGAACTGTTCTCAAGAATCCTCAAAACAGCCCTTTGAGGTAGGTTCTG
TTTCACTCCCAAAGAGAAGCGAGGTTGGCACTCAGAAATTTCACATCATTTGCCCTGGCTCACTGAATGA
ACTAGTGAATAGTGACGCCGGGTTCAGACCTTTCCGTTTTTCTTTAAAACTTGTGTTTCTTTCTACCAGA
ATGCAACTAGGGTTGGCCAGTGACCCCTTCTGTCCACCACGTGGGAGCAGCTCTCCTAAAGCTGGGCTCT
GCATTAAAGGGATTGTTTACAAATTCATTAAATTAAACATGTTTAATTCAGGGTAAAAACTGAATGATTT
AGAAGATCTTTTTTCTTTTGTTTTTTTCTTTGTTTTTTTTTTTTTTTTGAGATGGAGTCTCACTCTGTCT
CCCAGGCTGGAGTGCAGTGGCGCCATCTCTGCTCACTGCAAACTCCGCCTCCCGGGTTCCTGCCATTCTC
CTGCCTCAGCCTCCCGAGTAGCTGGGAGTACAGGCGCCCGCCACCGCGCCTGGCTAATTTTTTTTTTTTT
TTTTTTAGTGGAGATGGGGTTTCACCGTGTTAGCCAGGATGGTCTGGATCTCCTGACCTCGTGATTCACC
TGCCTCGGCCTCCCAAAATGCTGGGATTACAGGCGTGAGCCACCGCGCCAGGCCTTTTTTCTTTTATTTT
AAAGAACTGTTGTTATTTTTATTTCAATTAAATTGAAAAACACAGGGAAAAAATGCAACAACAACAAATC
CCAGGCAGGGCATGGCAACTCACGCCTGTAATCCCAGCACTTTGGATCACTTGAGTCCAGGAGTTTGAGA
CCAGCCTGGGCAACATAGTGAGACCTCAGCTCTAGAAAAAAAGAATTAGCTGGGCATGGTGGTGCATGCC
TGTGGTCCCAGCTACTAGGGAGGCTGAGGTGGGAGGATCGCTTGGAGGAGGTTGAGGCTGCAGTGAGCCT
AAATCAGTCATGCCACTGCACTCCAGCCTGGGCGACAGAACAAGACCTTGTCTAAAAAAAACCCAGAACC
AAAAAAAACAAAAAACCCCAACAAATCCCTCATAGCACCAGCACTTATTTTTGAAGACTTCCTGTACTTA
CACATATAAATATTTTGTACAAAAATGTTATGATATTATAAATATTTTTCTGCAGCTTCCTTATTTGACT
TAAGAATTTATGATGAACATCTTTCTGAGTCAGTCAGGAATATTCTTCATGCACTGTTAACTCCTCTGTT
ACTTTAACAATTTTGTGTGTGTGTGTGTGTGTGTGTGTGTGTGTTTTGATGGTTATTGTTATACTGACTT
TAGCCACTTTTCATCATTTTAGCCTTTAGGTCTCTTTTTTTCTTTGTGTGGTCTTAACCGAGAAATAATT
GCAAGCGAGGAACAAAGTCTTAAATTTGGTGGCACTCACCACCAAATAATTGCAGATGTTTGCCTTCCTT
CTACATAGAAAGAGCCCACTCTCATGCTGTATTTGTTCTTCTCATCCTCCCCTGTCTTATTTCTCTTTCA
TTTCGTTTAATTTTAACCTCAATACCAGACTTATTTTGTGCAGCCCTTGGCCTGCTGCGGAGTTATCTTT
GCCACTGGCTCTCTTTCCAGCCCCCAGTCGCTGATGTGTCTCTTGGCAGCTAGTGTAAATGATGGTGGTC
CTCTCATTCTGACGTGGGAGCACCCCAAGTCAGCAGTAATATTTGGTGTAACAAGTACCAGGGTGGGATA
ACCACATATCCCTGGACTGGGATTTTAAAATTGTCAGTTGTCCTGTCATTTGTGGTTTAGGGATCAACTT
GAGCAAGAGAAACTCCGTCTCTACTAAAAATACAAAAAAAAATTAGCCAGGCGTGGTGGCAGTTGCCTGT
AATCCCAGCTACTCAGGAGGCTGAGGCAAGAGAATTGCTTGAACCCAGGAGTCGGAGGCTGCATTGAGCC
GAGATCGTGCCACTGCACTCCAGCCTGGGCGACAGAGCGAGACTGTCTGAAAAAAAAAAAAAAGAAATTG
TATTTATTTCCTACAGACTTCTTTTTCTTGGTTATCACCTGACATTGCCTTATCAGGTATTTTATTATGT
ATTTTATTCCTTTTTATTATTTTTGGCTTAAATTTTCCTTTTAGTCTTTTACTTGGATCATTCTTTCTTA
TTTTCTCCTCCATATTTTGGATTCTTCAGACTTCTTCCCTTTTCTTAATCTTACCATTACCAGATTTCCC
TTCTGCTTAAATTTCATGATCCAGTTTTTCAGTGGATGGAGATAGAAGGCTGAGGTGAGGAAGGTGGGGG
TAAATACGATACTTGCTGAATTTCAAATATAAAACAAGATATTAAGGAACTTATTTCCTTTTTTATTCCC
TATATTTGGGAGACCATAGAATTGGCTGGCCGCTAATATCAAGTGGCTTTTGTGTGTAGACCCTTACAAA
GAAATGACTGATAAAGTCCCTACTTCTGGGAGTTCTGATAGTAAAAAGTGTGACATCCATGACACATACA
TTGCCAGTCAATGATGGATAGAAATGGACAGACTTTGAGGAGATGAGTTTTGAGTATACAGAGATACCAT
TTTGTGTGTGTGTGTGTGCGCATGCATTTGAGACAGGGTCTCACTCTGTCTCCCTGCCTGGAGTGCAGTG
ATGCGGACATGGCTCACTATGGCCTCGACCTCCCAGGCTTAAACAATTCTCCCACCTCAGCCTCCCGTGT
AGCTGGAACCACAGGCATGCGCCACCATGTCTGGCTAATTTTTTGATGTTTTGTAGAGCTGAGGTCTCAC
TATATTGCCCAGGCTGGTCTCGAACTCCTGGACTCAAGCGATCCTCCTTCCTCAGCCTCCCAAAGTGCTG
GAATTACAGGTGTGAGCCACTGCAGGGCCGCAGAGATACAGTATATTTGTGATACCATTAGTAACTTTTA
TTTGACAGAGGGTTAAGTATTGACTAAGGGATAAAACCTATTCTGGGGCTTAGAAAACAAGGTTTTGCTC
CAAGAAGGCGGTTAGATGTGGATAACGGTTTCAGTACTGAATTTGGAACCTTTTTACTAAAATGTAGTCT
ACATGTGGGTATCTAGCAATAGAGTCACTGTTAGTAAAGAACTATATATGTGACATACAGAGGAACCACA
TACAGTTTCTTAAAATAATATGTTCTTCATCTTTGAGGAAATCCTAGACAAAGCTGGAAAAATTTCAAGT
TTTCTTATTGCTCATTACAAATTATAGGGATTTTGATATTGTGCAGTTAAAAGTTCAGTAGATTTTCAAA
ACTAGTTATTAATTGACCTCGGTTCTGCACATTTTTGTTGCTTTGCAAGAGTACCCTTAAGGCTCAAATA
AAGTTAAAAGTAAGGTAAATGGTGTGTTAGACTAAAACTGTATTTTTTGTCTGTCAACACTTAAATAGAA
AAACAAACTAGCTTAAGAACATGACTGCAATTTAGGATCTTAAAACCATATATACCGTAGTATTTTTAAA
GCCTGCAACCAAAAAGTAGAATCATTGTGTTTTATAGCACTTTGAAATTCTGTCTGATGTTACTGTATGT
CTGTGCTGTCCTGATATGGCTTTTGGATTGCGTTTGTGGTACAAGATTGATTTCTTGTATTTGTATATTA
CTTGGCTGCACAGATGGTATCCGCTTCAAATCTACTAAATAAAACTAGAACAATGATTTTGCCAGTTGGT
TTTGACTGCTTTTATTTATTTTGGATGGCTGTAGTCTTTGTGAAGATGGCAGACTAAACCTGGAAGTGAC
AGCTAAGAACAGAATGGGTGGGCTACAGGTCTGCTAATTGATCTCAGCAGAACTCATGCTACTTCTCCCT
GGGTACAAGACGTTTTCTTCAATTGAATGTTCACAGGCCAGCTGAGCCATCCTTTGCTCCCATATCTCTA
TTGTCCTTGGCCCAGACCTTTTCTCATCCTCCCTGGGTTATCTCATCCATTTCCATAGCTTTAGTTACTG
TCTCTCTGTTGATGTTTCTACATCTATGTCTTCAGCTCAGGCCTGTTTCCTAACCTCTTCCTCACATACC
CACTGCCTATTGGACCTCTTCATTTAGATGCCCCAGGTCTCTTGTACTCACCATGCCCTAAGCTGAGCCT
ATCTTTTTATCCAAGTATGTTCCTACTCCTTTGTTGCCCCTTTCAGTGTATGGTGGCATAACACATCTAG
TTCTCTAATCAGAAACCTAAGCAACATCTTTACCTCCTCTCTATTCCTTTTTTTTACTATGTCTGATCTA
AATACTAAATACTATGTGTTCTCCCTGATAAGCATCTCTTATCTGGGTGCTTTTCTCTATTCCTTCATGT
GACTAGACTAGGCCTCAGTTACTGTAAGATGGTCTTCCATCTTACAGTATGATCTTGTCTCCCCTGGTGG
TCCATTTTCTATATTGCTTGTGTGATTTTTCTGAAACAGAAATCTGAGTATATCACTTTAATGCTAAAAA
TCCTCCAGTGACTTACCTTTGCTCTTAGAATAAAGTCCAGAGTCCTTACTCTGTTTTGTAAAGTCCTTTT
AAATCCATCTACTACTTGCTTGAGCAGGGTGACAGTCTTGGTAAAATGTACTTTTATAGCCAGAAGTCCC
CCTCACACTTAACATATCATGATGCTTTCCTTTCTTGTGAGAAGCGAGAAATTTTATTGTGACATTTTGT
CCATTAGTAAACCACACTCAGATCTGTGTGCCCCTCTTCCAAAAAATACATTATACATATTTCTACAAAG
AGAGAAAACATGTAATAGTTGTCAGGTTCTTTTGAGAAATCCTTTTGTAGAAGTTATGCTTGGTGTTGTT
TTAATGTTTTTATTACAAAGTAGAGAGCCTGTCTGATGAATCTTACACAAATAAAGTAATGGAGGTAATT
AGATAACACCACAGCCTTTCCTTTCTCTCCAGAAGAGGTAACAAGAGTCAGCATTCATTTGGAAGCAGTT
GCCCAGACATGCCAACAGAAGTTGTGGGCATCTGTATCTCCTTCTAAGAAAGCGGGTTTCCTGAAAACCT
TGGGAAAGGAAGGAAGAGAGGGACGGAGAGAGGGAGGGAAGATGCTATGTAGGTGGGCCAGTCACCAGCA
GAGGGATAAAAAATGGTGAGATCAAAATATAAAAGCACGAGTAGTGTATTCCTTTGGCTGGCCTCTATCT
CTAGATGCCCTAAAATGTTTATCTCCATTATTATAAGTCAGTGTAGATGCCTAGAGAAAGGAGGCATCCC
TGATTTGGGCAGGATTCTAAACAATTGCCTTTTGTATTGTTAGTCTACACTTTGCTGAAAGTCCCTCTTG
CCTCAGTCTGCTGTTCCCCTGGTGAGCCCCCACATATGGTTCAGTTCTCAGTGTCAGGTCTCTTTTTCTG
GAAACTTTCCTCAACACCCCAAATCTGGGTTAAGTGCTCCACTTAGTCTGCTGTACTATGTAACACTTCT
CACTCTGCAGTTTTAGTTTGTATTTTAAGTTCAGTGACTATGACTAATGTTATTGTTTGAAATGAAAGAG
ATTTTAAGAGTTTCTTCAAGAGGATTGACCAGAGTCCTGAGACATTTCTCCATTCCTAAATATATAAAAG
CCATGAGTTGCCTTTGAACTTCATTACAATTTTGAAAATTTTTAGACATATCAAAGGTATTCTGTAGATG
AGAGCTAGCCCGCTTCAATCCAGTTGTGACTATCTTGAGTGTGAATGTCAGTGTTCATGTTTGATGTTCT
TTATTTTGGTGTATACTCTAGGTGTAAAGCCAGCCCAATATTGTCCCAACACATGACTGTGTTTGTTTGG
CTAACTGAATACACCTTACCTGCCTTTTCTCCACTTTCTGTCTTCATATCGAAATCCATACACTTGAAAT
ATTCTAATCCATTAGATATATGTAATTTCCTCATTTATGCTTTACTGAAACCCTGTTGGAAAATTCAAAT
TAATTACCCTTAGATTAAAAAAAAAAGCCTAACAAAGCTCTGTGGATTTGGTATGGGAACATATCAATAA
ATTTTTTAAAATAAAAACCACATAATTATTTTTAAAGACAACATTTATTTTCAGATCTGTTGAGTTTTTC
CACCTATTTCTTCAAAGTTTTCCTTGTCATCCAAAGACATGACATTTGAGGATCTGTCTCTTTACTACTT
TTCTATTTTTCAGGCAATGAAACCTCTTGTTACATTTAATTAGTGTGTCTAGGTCATGATCCTTAGTCAT
TTTAAATTGGAGTGTGATAGAGAGGTTTTAATATATTGTGCAGTAGATCCTTGATATTTTCAGATGTAAA
AGGTTTGTTTTGATTTTTAAAATCACTTTATTGAGATATGATTGATATATAAAAAGTTGTACATATTTAA
TCTATAGAACTCAATGAGTTTGTGGATAAGTATATACCCATGAAGTTATCACCACCATCAAAACTGTAAA
CATACCCATCACCTCCCAAAGTTCCCCCTGCCTGTTTTGTTATTATTTTTTTGTGTATGGTAAGAACACT
TAATATAAGCTCTACTCTTTTAGTACATTTTTACTATACAGCATAATATTGTTAGCCATGGGCACTATGC
AATATAGTAGATCTCCAAAATTTACTTCTCTTTTAGGACTGAAACTTTGTACCTTTAACTATCACCTCTC
AATTTCCCCTCCTGCCAGCCCCTGGCAACAGCCATTGTACTCTTTTTTTTTTTTTTTTTAATTGATACAG
GGTCTCACTGTGTTGCCCAGGCTGGAATGAGTGGCGTGATCATAGCTCACTGTAGCCTCAACCTCCGGGG
CTCAAGAGATCCTCCTGCTGTAGCCTCCTAAGTAGCCAGGACTACAGGCATGCGCCATCCTGCCTGGCTA
ATTTTTTATTTTGTACAGATGGGGTTTTGCTCTGTTGCCTAGGCTGGTCTCAAACTCTGGCCTCAGATGA
TCCTCTCTTATTGTGGTTTTGATGTGTATTTCCCTGATGATTACTGATGTTGAGCATTGTTTCAAATAAC
TGCTAGCCATTTGTTTGTCTTCTTTGAAGAAAAGTTGGTTCAGGTCCCTTTGACCATTTAAAAGATCAGG
TTTTTTTGTTTTGCTTTGTTTTGTTTTGCTTTGTTTTGTTTTGTTTTGTTTTTTGTTTTGTTTTGGTATT
GAGCTGTTTGAGTTCCTTATATAATATATTCTGGATATTAACACCTTCTCAGATGCATAGTTTGCAAATA
TTTTCTCCCATTCTGTAGGTTGTCTCTTCACGCTGTTATTTTCTTTGCTGTGAAGACATTTTTAGTTTGA
TGGATTTTTAGTTTGATGTAATTCCATTTGTCTGTTTTTGCTTTTATTGCTTTTGTGGTCTTATCCAAAA
AATCTTTGCCCAGAAGACCAGTATTATGAAGCATTTTCTCTATGTTTTCTTCTAGTAGTTTCATAATTTC
AGGTATTACATTTAGCCCATTTTGTGTTGATTTTTCTTTTTTTTTAGATCAACATCTCAAACTTTATAAT
ACACTCACATTTATACACAAATTTCTGTTTACAAGTACAAAAGGCAAAGAACTGCAAAGAAGCTAAGCAA
CTGCATCATCAGCCACATTCAATCGAATTAATACTGTTGTCCCCCGTCCGTAGTTTTAATCCAGAGGAAA
TGGTAGATGCCGTCTCACACATGGACTCCACTGTTAGTTTTAGAAGTTGGAGACTGTGTGTGGTTCCGCA
GAAGTCTGGGTTTCTACAGAGCTGAACTGAGACTCCATCATGTTCCAGGCCATATCAGCCAGAAGAAAGT
CGTCCATTGTTGTTTGAGTTTCGTTGTTGGTGAAGAACAAGCTCCCCAGGGTTTCAAACCCAGAACTCAT
GGTCTGTGTTTCTATACTGTTCAACTGAACTTTGCTTTCTAGAGCAGGTATATCTTTAACAGTGGAGATT
CCTTCAGTTTGTGTCTCTGTGTCAGATGAATCAGTACTCATGGAAAAGCTGGAGTGTTTCAGAATACTTC
CCAGAGGCAGATGAGGGCTACTGTCTAAGAAAAAGTTTAAGTCTGTCTGTGTTTGTCTGTCAAACATCTC
AAGGCCTAAGAAGTTAGAATTTCCCCTACACCCATAGGACTGAGCAGAGGTATCTGCGAGTAAGAAGTCC
GTTTGAGTCTCTGTGTCCAGTGACTCCAAGACTGGCTCAGTGTTCATGGTGCTAAGTTCACTCTCTTCAG
TTTGAGTCTGGATATGTGAGGCCGAAAAGAACTCTTCAATATCAAAATTGATTCCAGGGTTCTGGGCTGG
GCCAGATGGGAGCTGGGTGTCAGGTCCAGGATTTGTGTTAGACAAAAGACTATGATGATCCAATGTCTGA
GCAGGCAGATTACTTGACAAGATGTTTTCCAAATCACTTAATAAATCTATGGTTTGGGTCTGATTATCTG
TCATGTTCTGTGAAGGAAGCATACTGTTCTGTGCACTGAAGTTTATAATTGGTGCACATTTCTCAATCTC
TTGATTTAAAGTCTTAGGCTCATTCTGAGGTAACAAACTATGAGTTACTATCTCTGCTACTAAACTGTTG
CTTATAATGTTACTTGTAGCAACATTATATGATGAATGAACACTCAAAAATGTCTCTGCACATTCCAGCT
TGGTCCATCTGTACATGGTCATCCGTTGGACTTTGTATCCCACTGGTTTGAGTTTCTCAGGAGACCCCAC
CCGACTGGAAACAGGTGTCCATAAATGCATCAGTCTGAGCAGCTATAGATGAAGTTACCTTAGAGCTGGG
CAAAAATGTCTGAGTGTGAACACTAATGGGAAGAGACACTTGAGAATCAAACAACAAATCAGTTTGAGAA
CAAGACGACACAGAGGAATCAGCGGTGGCCCACTGTGCAGAAGGTATAAAGTTTTGTGAGGCATAAGACA
GATCTGTCTGCACGTTGATTGAAGAAATGCCGTTCTTCTGACATGTGTTCCCTAGTTCTTGTAAAGTATT
AGATGGACTTTTACCCAAGTTCACTTGAACACCAGTACTTATTGGCTCAACAGCAACAGGATCAGCAATT
TAGAAAAGAAGTAGGCTCTCTTTAAGAGAGCAAGCCTCTGAATCTAGGCCGAGGATCAGGGTTCCTACTG
ACAAGGGCAGGCAGTAAGTGCACAGCCCCTGTGGCAGAGCCCTGATCAACACCTAACACCACAGGCTGGG
CTGAGGAGTCGGCTGTAGGCACAAAGACAGGCATGAGAGAAAACTGCATCACGGGTAGTTTAAGCAAAGC
CACTTTGGGCTTTGGTAAAAGCAACTTTTGAGGCTATCTCGGTGATGTTGTAAGAAAGAGTCTGCTTGTC
AGTGTTAGAGCCACAAGAGTCTTCAAAAGATGGTTCTAGCTTTATTTCTGAAGCTTCTAGTTCTTGGGTG
TCTGGTCTAGGGATTGGTTGGTTGTTAAATGATTCAGTGGTCTTGTTGGATAACTTCTGGTTTTGCACAG
TTTTCCATTTTCCTTTTCTTACTAGGTGGGTCCCTGTGTTCTGCAGGGATCTCGTGGCCAGTTCGGTAGA
TGTGAGACTGCAATGCTTTTCTACTGGCGTAGGGACAGCCGCATGTGCACCGGAAGGTCTTGCCACAGTC
CTCTGCAAGTCTTTTCAGGTCCCATTCTGTACCGTACGAATTGCTGCACTTGCTACATTTGTGCTTCTTC
TCAGCATGGATTTTCATAAAGTGCTGTTTTACGAGAGAAAACTGAGAAAATGGTCTGTCAGGGCCTCTGG
GGCAGCCTTCGATTGGACAACAGTAGAATTTCGGTACAGTTTTCAAATCTTTTCTTATTATTGGATTGAC
TGTGCTATACTGCATGCGGTGGCTCTTGACCAGGTGCATGTTGAGCGCGGGGCTGTTGGGCAGGATCTTG
CGGCAGCCGCGCACGGTGCACAGGATGTTGGGCCATGTGGTCTGGGACAGCTCGCTCACCTACTGCTGGA
TCAGCTCCCCCGCCAGCACGGAGGCCGCGGGCTGCTGCGTCACCCCCGCGGGCCGCGGCCGGCTGCCCCT
CAGTCGGGGTCCCGGGGGCACCCACGGGCCCGAGGCGGCGGCGGAGGCTCCTCTCGTGGCCGCCGGGACG
GCGCGGGCACCCGCCGCCAGAGCCGTGGACCCCGCCGCCGCCACCGCCTCCGAGGCCGCCATGGCTCCCG
CAGGGCGGCCAGGCCCAGCTCGCAGTTGTGTTGATTTTTCTAAGTGGTGAGAGATAGGGTCTACCTCCGT
TTTTCTGCATGTGGATATCCAGTTTTCCTGAACACCATTCATTGAAGAGACTGTCCTTTCCCCAGTGTGT
GTTCTTGGCGCCTTTGTCAAAAATCAGTTGACTGTAAGTGTGTTCTCTTTTCTGTTCCATTTGTCTGTGT
GTTTTTATGTCAGTACTCTACTGTTTTGCTTACTATAGCTTTGTAGTATATTTTGAAGTCAGGTAAGATT
GATGCCTCTAGCTTTGTTTTTTTGACGCAAGATTGCTTTGGCTCTTTGGGGTCTTAATATATATATATAT
ATATATATATATTTTTTTTTTTTTTTTTTTTTGAGACAGAGTCTCACTCTCTCACCTAGGCTGGAGTGGA
GTGGCGCGATCTTGGCTCACTGCATCCTCCACCTCCTGGGTTCAAGAGATCCTCCTGCCTCAGCCTCCCG
AGTAACCGGGACTACAGGTGCCTGCCACTATCCTCAGCTAATTTTTGTATTTTTAGTAGAGATGGGGTTT
CGCCATGTTGGCCAGGGTGGTCTCGCACTCCTGACCTCAGGTGATCCACCTGCCTCGGCCTCCCAAAGTG
CTGGGATTACAGGCATGAGTCACTGTGCCCAGCCTCCATACAAATTTTAACGGACTTTTAAAAGATACGA
ATTCTTCCAATCTGTGAACATGGGGTATCTTCCCATTTTTTGTGTGTCTTCAATTTTTTTCAATATTTTA
TAGTTTTCATTGTAGAGATCTTTCACATCCTTGGTTAAATTTATACCTAAGTATTTTATTTCTTTTTTTT
TGGCAGGTATTGTAAATGGGATTGCTTTCTTGATTTATTTTTCAGATAGTTTGCTATTAATATTTGGTGT
ATAGAAACACTAATGATTTTTGTTTGTTGATTTTATATCCTGCAACTTGACTGAATTCATTTATTAGTTC
TAATAGTTTTTTTTTAGTGGAATTTGTAGGGTTTTCTATGTACTGTGTCATCTGTAAACATAGACAGTTT
AACTTCCTCCTTTCCAGTTTGGATGCCCTTTCTTTCTTTCTCTTACCTAATTGCTCTGGGTAGGATTTCC
AGTGCTGTGTTGGATAGAAGTGGTAAGAGTGGACCTCGTTGTATTGTTCCTGATTTTAGAGAAAATGCTT
TTAACTTTTCCCCAGTCGGTATGGTTAGTTGTGGGTCTGTCACAGATGGCCTTGTGTTGATGTACATTCC
TTCTATACCTAATTTGTTGACAGTTTTTTTTATCATGAAAGATGTCGAATTTTGTCAAATGCTCTTTCTG
CATCTATTGAGATGATCATATGATTTTTATTCTTGGTTCTGTTAATGTGGTATATCATCTTCATAGATTG
GCGTATGTTGAATCATCCTTACAACCCAGGGGTAAATTTCATTTGATCATGATATGTGATCTTTTTTTAT
GTGTTGTTGAATTCTGTTTGCTAGTGTTGTGTTGAGTGAGGACTTCTGCATCTAAGTTTATCAGGGATAT
TGGCTTATGATTTTCTTTTTGTGTCTTTGCCTCCTATTCTTAGCAGTGTTCCCTTGAAAGCAATTTGTCT
TTTTATAATCTCTGTCTGCTTTTAGGACTTCCTCTTTGCCTTTAGTTTCTGCAGTTTTACTGTGTATTTT
ACTTCAGAGGAGTGTGTTTTGTCCCATTTTCCAGAGGTCTTGTTGGACTGAGCTCTGGTTACCTACCCAC
TCTGGTAGTTGGCTTAATAACCCTCCTTTCATTGGCTGTGATTCCCTGTGTCACTTCTCTACTTTCTTAA
TGGTGTTCCCTTTATTGTCCAAATAAACTACTCTACTCAAATCCCTTTTTCAAGATCTCCTTCTTGGGAG
TAATAGGGCTTCTTCGATCTGAGGATTAATGTTCGTGTTGATCACAATGGAATAGTGATATTGCTGACAG
AAAGATATCATTGGTTATACTTGGAAGATCCTCAGTTTCCCTAATGGCAAAAGACTTGGGAATCTGTGGA
TCAAGATGGAGCATATATGATCCCAGACAGAAGTATTGTTTCTCAGGTCCCCCAGAGTTCCCCAAAGGAT
GGATTTGCATGGCAAGAGAAGCTCCCTTATCACTTCTAATGGGGTTTTATATGGTTTTGGTCAATTACCC
TTCCACTGCTTGAACATTACTCTTCTGGAACCCAGTGACATGTGTGCCTACTCAGGAGGTAGAATTAACT
GATTCACAACAATTGCCAAACGTTTTTTAGAGTTTACTGCAAGGTTGGATGACATGAAGACCAATTTTCT
GGAATCAGTCTTTCAGAAGATGTAAGCCTCAGGCATGTCTGATTTCTATTCTAAAACGTGTGGCTGGGCA
CAATGGATCATGTCTGTAATCCCAGCACTTTGGGAGGCTGAGGCAGGAGGATCACTTGAGCCCAGGAGTT
TGAGACCAGCCTGGGCAACATAGCAAGACCCTGTCTCCACAAAAAATAAAAATAATATCAGATTAGTCAG
GCATGGTGGCATGTGCCTATAGTCTCAGCCATTTGGGAGGCTGAGGTAGGAGGATCCTTTGAGCCTTGGA
GGTCAAGGCTGTAGTGAGCCATGATAGCACCACTGTACTCCAGCCTGGGTGACAGAGCAAGACCCTGTCT
CAGTCAGTCAATAAAATATCATACAGTTATGGTTAAAAACTAAAACAAATTTTTAAAAATTTGAATGAAC
AATAGAGAATGTATTAATATGCTTTGAATTTATATTAATTTTGTGGAGAAGAATCATTTAGAATTTTAGC
TCAATCATAAGCCTGTAGATGATGACACATGAAATTATTATCCCCCCGTGAAATACAAGTGTGAAAGATG
ACTACCTTTTTGTGGTATGATTTCTATATACTATACTCACGCGCACTTCAAGGTTCAGTTGTGTGGTTTT
TCAGTTCTTACTCCAGCCACTGGTTGTGCAAATACTCCGTGATTGTGTAAAAATCATACCTGTTAATTTA
TATATACTGTTACTGGCAGGAGACAGACAGATAGTGCTATTTATTAGAATTATATGTGCTTCTGAGCATT
AGTTACCTAATCAGGTATTTTAAAAGTTCAGAAAAAAATTTTTAAGTCTGAACCTTATGTAGTAACTCTG
AAAAACTATAGAAAACAGAGTAATAAAATATAAAAGTAGTAGATTGAAATGATACTCTTCTTATTATCAT
TATTTCTAGAGACAGGGTCTTGCTCTTTTGTCTAGGCTGGAGTGCAGTGGCATGATCCGTAGCTCATTGC
AGCCTCAAACTCATGGGCTCAAGCAGCCCTCCCTCCTCAGCCTCCCAAGCAGCTAGGACTACTGGTGTGT
GTCACCACACCTGGCTAATTTTTGTTTGTCTGTTTTTTTGTGAAGATGGGGTCTCACTGTTAACTGAGCT
GGTCTCAGAACTCCTGGGCTAAAGTGATCCTCCCATCTCAGCCTCCCTAAGTACTAGGGTTACACGCTTG
CACTACCAAGCCTGGTCTGAAATGAGACTCTTAAAGGAAACAGGAAACAGTGAACAAAGAGAAGAGTCAT
TGGAAATTAAAAAAAAAAAAAAGTGAAATGCCCTATGTGTGATCAAATAGAAGCACTGGATTCAGATTAT
CAACAAACTAATCAAGATGGAAGAGCATAGGAAATTTCCCCTGGCAAGAGCTTGGTAACCAAGACGTCAG
GGGCCAGAGTGAAACATGCTGAATGCATGAGCCAAGTGCTGGGACTCAGTTGACCTATAAGAACAAATAA
AAAGAAATCCATGTAAATCCATATAAAACACAAAACCATGGACACTTGTATTAAAGTATGGCTTGTCAAT
AGAATTTGCATCAGTGCATGCTAAGGTTAAGTCACAGTCAAAGCCTATTCAAAGAATAGCCTAGCGGTTT
TCAAGTAGCTGGGCAGGTAAAGAAAATCCCTTGACATATATAACTCTTAAGATCTGCTGTAACAAATAGT
TATTATACTAGCCAAACCAAGTTTAATTTTCTCCTATATATGGTCTTGGTAAAGGCATCATGTTATATAG
AACAAACATGATCGACTTGGATAATGAAATTGTAAATAAAATCCTGCTTTACAAATTATTATTGCTGAAA
CAGCCTGTGTTTTAGAGAAAGCAAGAATGTTTTTAGATAAAAGAAATAATTATTCCACTTATTAATATTA
AAATTAAATTATAGCATTTTATTTTAATGTCAGAAGTACTTGTTCTGTACAGAGATTATGAAGAACTGGG
ATCATGAATCTGTTGCTGATAAATAAAATTTCAGAGGTCTTGGGATAAATTATGAATTCAGTTTGGGAGA
ACAGGGGTTCCTTATCAGCTAGAAAAACTGATTTTACTACCATATGGCAGTGGTAAAAAAAAGTCCATAC
CAAATTCCTTATCTTGGATATGTATCTTGAATTTTTATGCAACAGAACAGAAATACAGTTTTGTTCAGGA
AAAAAAATTGCAGAAATACTACTCTGTGCAGCTCCTGTCTAGAACATGCCTGCCTTTTTGTTGTACACAA
GAAAGAGAATTTTCTAGTTTAATTCCTTGAAGAGCAATTTGAATGGTTTCCACTTGGGAAATTATGTAAT
TTCATATTAAATAGCAGGTGGTAAGGTAGTCAGATGGAAATGTTCAGTTCATCCAGCATCATCAGATTGA
TTTGACAAATGGAATTCTTTTAACTCTATGGAAAGCATTTTTCTCCCTTTATCCTTTGGCAGAGGCAGAC
ATTGTCTGTGGTTTCCCCACAAAAGTTTGAGTGGGTAAACTTTGGATGTGTAAATATAAACAGCAGACAG
AGACATTGCCTCTGACAGCTGGAAGGATCCACTGAGGATCTTAGAAGGAGACTGCTGATATGGATGATGT
TGGATAGAGAGACTTGAGGGAGGAATTAACCAATGTGAACATAAATGAGGAATGCACATGCTTAGAAAAA
ATGATAGTTCCTCCTTAAGTTTAGGTTGAGGTTGTTGAAAATTTTTGCTTTTGATATGGATAAAAACAAC
AAAAAACTGACTTTTATGGATAAAAACAACAAAAAACAATAAAAAACTGACATACCATGTGGATGAGCAC
ATGGTATGTTTCTTGGTGAGTGGCTCCATCTCGTACATGATATGTAACCTTGGGCAAGATATTTGGGCAA
GATATTTACTTCTCTTTTCCTTAGTTTCCACACCTGTATAGTGGGATTGTAGGGACTGAGATAAGTTTAC
GGTGCTTATAATTGTCCTTGGCATAGAATAAGCACCATGTAAGTGATAGCTGCTATAAAATAATAATAAT
TATTATTTTGGTGGATTGAGAAATTCCCTTAAGGAGGAAGCTATTTGCTTCCTCTAATAACCTTAGTATA
GTTGATGTATTTATATATAAATGAGATACAGAATTCAGGAGCAAATTATTTAGTGTTTAAGATGATTTAT
AAAGATATGTTCATATTACCTTTTAAAAATTAAATTGTGAAGAATACTGCAGGTGCCCGCCACCATGCCT
CACTAAACTCTGAGCCTTTGCACGTGCCAGGAAAGTGCTGTGGCTGGACACTGGGTCTGATGATGCTCCC
CTTTTCTTTGCTTGAACAGTAATCAGCCCTCGAGACTCTTCTGGTTTGTGAAATCTTTGTGCCCCCACGT
TATGTGTGTGTGCTGCCATTAGATCAATTTCACATTATTATTGGAATTTTTGTTTGCATATCTGCTACCC
CACTCAGTTGTGTGTTTTCCAGTCTACTATAAAATGTTTCAAAGATTAAAAATTACATCTTTTCATTAGC
CACAGTGCCTGCTGCATAGTAGGTGTTTTTAGGTAAATGATTGAATGATAAATAAATCATACCGGTCATG
ACAGTATTCAAAAGAAAACAGTTCTTCTCTTGTTTTTCCTGTAAGGAGCAAGGAAATCTTTCCTGGAAGC
CCCTACACCAGACATCTCCACGTATTGGCCTAGGATACAGTCACTGACAAGGGAATGGAATTTCCCTAGT
TGGCTTGGAAAAGGCAGTTTGTGTTTAACCACAAAGATCACTGCACTGGTTACAAAAATAATACATGATT
ATTATAGGAAATTTATAAATTAATGAAAAGTACAATAAAATAATCTGGCATTACACAAAGAAAACTGTTG
TTAACATTGTGGTGTTTGCTTTCCAGTTTTTGGTTGTATATGTATATGTTTGTGTGTATAGAAGAAAATT
GCAATCATATTGTATATAGCTTTTTATAGTCTTACATTTAAGTCTAACATAAGCAGTTTTCCGTACCATT
AAGTCATATTTTTCTGAAATTAGACTAATGGCTTAAGTTCCTTCATATGGCTGTATTACAAATTATATAA
TTCATTGCTTATTGTTGGAGATATAGATTGTTAGCAGTTTTTTACTATTGTAAATAAATATGCAGTGAAC
ATCCTTTTATATCAATCATGTACATCTCTGGTTATTTCCTTACACTAAATTCCTAGAAGTGGATTAATGA
GTGAAAGGGTATAAACACATTCAAAGTTTTTTGATATACTTCTCTGATTATCTTTCAGAAAACTTGGACC
AATTACATTACCAGAAACCTGCATATGAGTGGCTGTTTAACTGTATTCTTGCCAGTACTGGGAATTTTTT
TTTTTCAAGACAGGATCTTGCCCTGTTGCCCAGGCTGGAGTGCAGTGGTATAATCATAGCTCACTGCAGC
CTCAGCTTCCTGGGCTCAAGCAATCTGTGAAGTATTTTTTAATCACTGATTTTATAGTCAAAAGTGGTTT
CACTCTGCTTTTTTGACTTGTAGTGTTATTTTTCATTTAGTCAGCATTTAGTAGATGTTTGTTGAAAGAA
GAAATCCGTGTTTCTTGATATGTATATTGTTTATTCACATCTATTATTGGCATTATTATTTTTGCTTTTT
TATTGTGATATACAAAAAGTACATAAGCTGTACTAATGTTAAGTGTAAAACTCCATATAATTTATGGCAT
TCTATTTTCAGTTTGCTGTTTTGGATATTAGTTTGTTTAGTTTATAGTATTATAATAATTTATAATATTA
TCTCAGCCAAGTCTGTTGGGTGAGATTTATTCCATCATAGTTATATTCTCCCTAGGGCTCCTAATTACAT
AGAGACTTCATTTTTGGTTCTGACTGAGAAATAAATTATTTCTTTCTCAATTCCTTTATATTATTTATTA
CATGTCTATGGTTTTTTTGCACAATCCTTCTCTTTGGCAACATACCGTTTTATTTATCTTCATGTTCTTC
TAGATATCAAAATCTTTTTTTTTCAGTATTTAGAAGAACCATTAAATTATTTCCTTTTTATGCTTTCTCC
CTTATTCCTTGGCTCAGATTTATTTTTTCTGTGTGTGTGCGTGTCTCTCTCTGTGCGTGTGCGCATGCGT
ATGTTTGTGTGTGTGTGTGTGTGTGTGTGTGTATGTGTGTGTGTGTGTGAGAGAGAGGAGTCTTAGAGTT
CAAGTAAATGATACTTTCCTGTGTTATTTATTAACAGTTACAACTGCTGTTGTTAATATTTATCTCATTT
AATCTTTTTTTGTCTGGTAAGACTGGTACATATCATTGTGTTTTTCATTTAGAGGGAAGTGAAATATTTT
GGGCCATACTGTTAATAGTGCTGTGTGGATCAGAGCTGAGATTAGAACCCAGTATGCTAGTGATATGTTT
GGATTTCAAGTTATAAACTGTACTGGGTTTAGTCTTGCACATAAAAAGAGGCTGAACTGAAGTTTCCAAG
ATGTTGAGAAATTTACAGTTTATTTAAATTTGAGTCTTGCTCTCAAGTACACTCTTTTCCCAGTTATTTG
CAGCTAGAAAAGCAAAAGTCTTCAAGTATAATTTAATATGACCAATAGGAACCACATGACTTTTACAGTC
ATTTTGTTGCGTACTAGTGTTTATACAACTCATGACTTGACAGCCGACCAAACTGTCTTATCCATCTCAT
ATTAACATATTGTGAATGTAGTAGGAACTGGCTTAGCAAAATCTTGAAACCTCTGACTCTAATATTTGTG
GGATAGTAAAGTAAACAACTTTGGGGCTTTCAGGCTGTGTGATGCACAGCCCGTTCAGGACATTAATGCT
TATTCTCTCTGTTGACTCCATTGCTTTTGTGAACAGTTTTGCCGACAGGTGTTTAGGCTTGCCAAGAGAG
ATTATTTCTAGCTAGTAGAGGACTCTTGGAGAAAGACATTGGTTTCTACTTAGAAATACAATAACTAATG
ATAATAACTGACATTGAATGGGTGTTAAAATGTGCCAGGCACAATTCTAAGTGCTTTCTGTGTAATTAAT
GTATTTTATTATTCACAGCAACCCTACTAGGTTTTTCCCATTTTACAGATACAGAAACGGAGGCATAGAC
TGGCAAAATAACTTCCCAAGGTCACATAGCTAGTATGTTATAGAGTAGGGATTCAAACTTCGGCAGTCTG
CCTCCAAGTCAGCCTTCTTAATCACTCTGTTATGCTGACATTTATTAGATGAAAAGAAAGAGGCACAGGT
GAGTCATAAGTGGCTGTTTGTGTTTTTGGAAAATATTTGACTTTATGCCTACTGGCAGCTTGAAATAATT
AAGCTGCTACCATTCATTAAATTTTTATTTTTTTGTAAAGTGAGTTATTTGCTTGCATGTAATATTATAA
TTGGAACATAGTGAGTAAAGTATACCCTTTTATGTTTGTACAAATTTAGGGTAAGCCTGTATTTTCATTA
AAAAAGTGGTCTTTGAGAAAGAAAGTGTTTGAAACAAAACTTTGAAGAGTGGGTTCAGAATCAGGGTGAC
AGCCACAGAGTAGTAAGTTATTTGGTCAATGCTCTGTAATAAATAGCAGAGACTGCCTTATTGGTCCTGT
GCGCCATCTTGAAGAGCTGAATGTTGCAGAAATGGCAGGCTAAATGTTTTGGAGTAACCCAAGCTGACTC
TAGGCAGAATTTGTCTTTGGCATCTTCTCATTTTGCCTGTCACTGGCACCTGTCTCATTCACCTTTTGTC
ATTGCCAGATATGGTATGGTGCCTAACAACCTTTACTAACTATCAACAAGTCCAGCTGGGTGTGGTGACA
TGCACCTGTAGTCCCAGCCCCTGGGGAGGCTGAGGAGGAGGATCCCTTGAGCCCACGAGTTTGAGGCTGT
AGTGAGCTATGATTGTGCCACTGCACACCAGTCTGGGCAACAGAGGAAGAGCCATCTGTTCAAAACAAAA
CAACAACAAAACAAGTTTCTCATAAAATATTAATTAATGTTTAATAAAAGGGATATCCTTTCTCAGGGTA
TTTGCATTTAATAGATCATTCTCAAAGTTTGGGCTTTGGGTTATTTTTGAGAGTTGGCATTTATTGCTAT
AAACTTCCCTCTTAGAATTGTTTTTGCTCTATCCCATAGGATTTGGTATGTTATGTTTCCGTTTTCATTT
GTCTCAGGATATTTTTAAATTTCTCTTTAAATTTATTCATTAACTCATTGTTTAGGAGCATGTTGTTTAA
TTTCTATGTATTTGTAAAGTTTTCAAGGTTTCCCCTTTTATTGATGTCTAGTTTTATACCATTGTGGTCA
GAAAAGATACTTGACAAGATTTTGATACTCTTAAATTTAAAAGACTTATTTTGTGGCCTAACATATGATT
TATACTGGAAAATGATTGATGTGCAGTTGAGAAGAATGTGTATTCTGTACCTGTTGGATAGAATGTTCTG
TAAATGTTTGTTAGGTCCATTTGGTCTAGGGTACAGTTCATATCTGATGTTTCTTTGTTGACTTTCTGTC
TAAATGATCTGTCCATTGCTGAAAATAGGGTGTTAAACTCTTCTACTAGTATTGTGTTACAATCTATCTG
TTTCTTGAGATCTATTAATATTGGCTTTATGTATTTAGGTGCTCTGACATTGGGTGCATATACATTTACC
CAACATCAGAGCACCTAAATATGTAACATATCCTTTTGCTGAATTGACCAACCCCATTATCATATATTAA
TATAATGACCTTCTTTGTCTCTATTTACAGATGACTTAAAGTCTATTTTATCTGATATAAGTATAGCCAC
TCCTGTTTTGGTTTCCATTTGTGTGAAGTATCTTTTTCCATTTATTTACTTTCAGTCTATGTGTATCCTT
ATAGGTGAGGCAAGTCTTTTGTAGGCAGTATATAGTTGGGTCTTGTTTTCTTTTTTTTTAAATCCATTTA
GCTACTCTGTGTCTTTTAATTGGATAATTTAATCCATTTACATTCAAAGTAATTATTGGTAGGTAAGGAT
TTACTACTGTCATTTTATTAATTTTTTTCTAGTTTTGTAGATTCTTTTTTTCTTTCTTTCTTCCTTTGAG
GTTAAGTGATTTTCTCTAGTAGTATATTTTGATTCTTTGCTTTTTATTTTTAGTGTATCTATTATAGGCT
TTTGCTTTGTGGTTACCATGGGACTTATAAAAGTATGCCTTATAGTTATAAAAGGTTATTTTACACTGAT
AGCAACTTTGATCGAAAAGAAAATACTCTTTAACTCCACTCCCCCATTTTGAGCTTTTGGTGTCAAAATT
TATATCTTTTTATATTGCATGTCCCCTAACAAATTATCGTAGCTATTATTTTTAATTGTGTTGTCTTAAC
CTTCTTAAAAAAATATATGATGTACATACCACCATTACAGTATTAGAGACTTCAGAATTTGACTGTGTAC
TTGCTTTTATCAGTGAGTTATATACTTTCAGATGTTTTTGTGTTGCTCATTGCTATCCTTTTCTTTCATC
TTGAAGAACTCCCTTTAGCATTTCTTGTAAGACAGGTCTGGTGCTGATGAATTCCATCAGCTTTTGTCGT
CCAGAAGTCTTTATTCTTTTCTTCATTTCTGAAGTACAGCTTTGGTAGGTGTAATATTCCTGGTTGATAG
TTTTAATTTTTTTTTCTTTAGGACTTTGAATTTCTTCAGGACAATGTTTCTGCTTAGAGGTCTCTTGTTA
GGCATATTAGTACTCCCTTATAAATTATTTGCTTCTTTTCTCTTGCTGCTTTCAGGATCCTCTCTTTGTG
CTTGATCTTTGACATTTTGATTATAATATGTTGTGGGGTAGTGTTATTTGGATTAAATCTGACTGGTTGC
CCTTGATCTTCCTGTACGTGGATAGCTATAGCTTTCTCCAGGTTTGAAATACTTTCTGTTATTATTTCTT
TAATAAGCTTTCTACTACTTTCTCTTTTTCAAGGTTCCCCCTTGAACACCAGTGACCCACATATTTGCTC
TTTAGATGTTACTCATAAATCCTGTAACTTCATTCCTTTTTCCTTTTTCACCTTTAGTTACGTATTTTCA
ATTAACCTGTCTTTGAATTCACTGATACTCCTCTAATTGAACAATTCTGCTATTGATGCTTTCTGTTGGA
TTTTTTATTTTGTTCATTGTAGTTTTTAGCTCCTGGATTTCTGTTTGATTTTAAAAAATTATTTCAATCT
CTCTGTTAAATTTCTCTGATGAATTTCTGAATTGTTTTTCTCTACTTTATTGAAGTTAGCCAAATTTCCT
TAAAACAAGTATTTTGAATTTCTGTCAGGCAGCTTTTCTATAATCCTCTCTTTAGGATCAGTTACTGGCA
GCTTATCTTTGTCCCAAAGCATGTTTTTCCTCCTTTGGGCCAGCTGGTTATTTAGTAGTGCCTTGTTCAG
TTTCCACATTTGTGAATTTCCTTTGAAACTTCCTGCATTTATTTTTAATTGCTTTTTTTGTCATCAGAGC
ACATTTGTTATATTACTAATGTTCTTTTAAATATATGAAGACTCATTTTATGACCTAGCATATGATCTGT
TTAGAATATTCCATGTGATCTTAAGAAAAATGTATATACTATTGGGTGGAGAATTCTATAGATGTAAGTT
AGATCTAGTTGGTTTATAGTGTTATTCAAGTGTTCTGTTTTCTTGTTGATCTTCTTTCTAGTTGTTTTAT
CTATCACTGAAAATGACAGTTTGAAGTCTCCAGCGATTAATATTCAATTGTCTGTTTCTCCCTTTCTGTC
AATTTTTGCTTCATGAATTTTGGTCCTTAGTTGTGTATGTTTATAATGGTTATATCTTCCTGATGGATTG
ACCCTTTTATGATTAACAAAATGTCCCTCTCCATCTCTAATAGCTTTTTGTTTTAAAATTTGTTTTTTTT
AATATCAGTATAGCCACAATTAAGTTTCTTGTTTTTGTATGTTTGCATGACATATCTTTTCTATCCTTTT
ACTTCCAATCTGTTTGTATTTTTCACTCTAAAGTGTGTCTTCTATAGACAGCATATAGTTAGATCATGTT
TTCTTAAAGTCTGACAATCTTTGTCTTTTGATGGGAATTTTTTTTTTTTTTTTTTTTTTTTTGAGACAGA
GTCTTGATCTGCCACTCAGGCTGGAGTGCAATGGCACGATCTCTGCTCACTGCAGCCTCCGCCTCTCAGG
TTCAAGCGATTCTCCTGCCTCACCCACCCGAGTAGCTGGGATTACAGGCACCTGCCACCACAGCCAGCTA
ATTTTTGTATTTTTAGTAAAGACAGCGTTTCATTATGTTAGCCAGGCTGGTCTTGAACTCTTGACCTCAA
GTGATCCACCCACCTTGGCCTCCCAAAGTGCTGGGATTACAGGTGTGAGCCACCGTGTCCAGCCTGATGG
GATTGTTTATTCCACTCACATCCATGATATTATTGACATAGTTGGCTTTATGTCTGAACTTTTACTTTTT
CTGTATATCTCGTCTTTTTTTTGTAACTCTGTTTCTCCTTTTATTACGCTCTTTTAAGTAAGTATTATCT
GTTTTTTTTTTTTTTAGACAGGGTCTCACTCTCTTGCCCAGGCTGGAGTGCAGTGGCATGGACATGGCTC
ACTGCCACTGTTGACCTCCTGGGCTCAAGTGATTCTCCTTCCTTTGCTGCCCATGAAACTGGGACCACAG
GCATGCACCACCACACCTGGCTAATTTTTCTATTTTTGATTTTTTTTTTCTTTTTTTTTCTTGAGACAGA
GTATTGGTCTGTCACCCAGGCTGGAGTGCAGTGGTGTGAGCTCGGCTCACTGCAACCTCTGTCTCCCGGG
CTCAAACTATTCTCATGGTTCAGCCTCCTGAGTGATTAGGACTGCAAGCATGCACCACCATGCCCAGCTA
ATTTTTGTATTTTTAGTAGAGACGGGGGGTTTCACCATGTTGGCCAGGCTAGTCTCTTAAGTCTTGGCCT
CAAGTGATCTGCCTGCCTTCACCTCCCAGAGTTCTGGGATTACAGGTGTAAGCCAGCATACCCAGCCAAT
TTTTTTTATTTTTTTGTAGAGATTTTTTGTTGCCCAGGCCAGTCTCAAACTCCTGGGCTCAAGTGATCCT
TCTGTCTTGGCCTCTCAAAGTGCTGGGATTACAGGCATGAGTAGTAACTGATGCCTGTAAGTGAATATTT
CATAATGTAGCATTTTAATTTCTTTAATGATGTTTTCACTATTCTTTTTGAATTTTTTTTTTTTAGTGGC
TACTCTAGAGTTTGCCGTAGAATAATTACCGTGATATATAGAAATGTTACTCCTATACATATGATTTTTG
ACTTATGTCCTATACATATGATTCTCGACTTATGATGGAGTTATATCATCATAAGTTGAAAATATTGTAA
GTCAGAAATTCACTTAATACACCTAATGTAGTGCACATCATTGCTTAGTGACACCGTGCACTGTAGAGTG
TTACTTGTTTACCCCTGTGATTGCTGGGCTGACTGGGAGCTGTGGCTTGCTGTCACTGCCCAGCATTGTG
AGAAAAGCATACTTGAGAAGAATGTATGTTCTGTTGTTGGGTGGAGAGTTCTATAGATGTCTGTTAGGTC
TGCTTGGTTATAGTGTTATTCAAGTGTTCTATTTTCTCGATCTTTTTTCTAGTTGTTTTATCAGTAGTTA
TGGTATCACATATCAGTAGCCTGGGAAAAGAGCAAAATTCAAAATTTGGAGTATGGCTTCTACTGACTGT
GTATCACTTTCATATCATTGTAAAGTCAAAAAATTGTAAGTCTAACCTTCATAAGTTATGGATCATCTGC
ATAGCTTTATTCCCTTCTCTTCCTTTTTTTGTGACATTATTGTTATACATATTACCTCTCAATGTTATGA
ATTCAACAATGTTTTAATTATTACTTTAACATGTGTAGTTACTTCCTTAGCCCAATACAGCTTGGCTGTT
TAACTCATTTGCACTGTGTTTGGCAAATATATTTACATATCTTATTCCTTTCTATATGTTATAGGCCCAA
CAGTACAGTGTGTACATATCATTTTATGCAATTCATTTTTATGTCAGTTATGAGAATAAGAGAGAAAAAA
TGTGCATTTATAATTACATAATTATCAGTGTTCTTTTATTGTGTGGATTCAAATTACCTTCTGGTATTAA
TTCACTTTTTTTTGCAAAGTGGGTTTTCTAGCAACAAATTCAGTTTTTGTTCTTCTGGGAAAGTCTTTAT
TTCACCTTCATTTTTGAAAGGTAGTTTTGGGGGATATGGGATTCTTGGTTGACAACTTTTGAGCACTTTG
AATATGTTTTCCCATGTTTTTCTGGGATCTTTTGTTTCTGCTGAGAAGTCACCTGTTAATCTTATTGGGG
TTCCCTTGTAAGTAATGGGCTATTTTTCTATTTCTGCTTTTAAGACTTTCTCCTTGTCCTTGACTTTCAG
CATTTTCACTACAGTGTTTTTGTTTGTGGATCTCATTGCATTTATCTTATTTGGGGTTTGTTGATGTTCC
CAAGTGCCAATAAATTTGGGGAGTTTATATGTTTCTTTTTAAAAATTATTTTATTTTTAATTGGCACGTA
ATGTGCATATTTATAGGGTGCAATGTGATGTTTCAACACATGTATATATTATGTACTGATCAAATTAGGA
TGATTAGCATATCCGTCATCTGAAACATTGTTTCTTTGTGGTGAGAACTTTCAAAATCCTCACTTCTAGT
TATTTTGAAATATGCATACATTATGTTTAACCCTTTTCCCATTTAGGAAAAAAGGTGCAGCTCACTACCC
AGCGCTCATTTAATTTTACATTAATGTGCTCTTTGAGGCTGAAGCAAATCTGACAGATTTTCAATGTGAA
AATAAAATATAAAAACTGTTTTTGGAGTTATTTCTAAACAGAACTAACATCAGAATTGTCTGAATCATCA
GAATCATCTATTTCAGAAAAATTGAATTGAAATGGATCTTTGGCTAATAACTGTTCAAGAACAATGTTAC
CATCATGCGTAGGAATGCTGCATTTTCTAGGATTTGATATTTTCAGCGATTGAGAATTACTATATTTTGT
AAATGGAAATACCAGTGCTAAAAACAGAATGTTATAAATAGAATGATGTTTTTTGTTTCCAAATTCAATG
TACTAGAGCTTGCGAAAATAGTAGTAAAAGCGAGCAAAGTTATCTCAGAGTAGACGCTGCAGCCTCATGT
ACTGTGGGCGAGTATTCTCTGGGCAAACGGGAAAAGGGTTAACTGTAGTCAACCTACTGTACAATAGAAT
ACAAGAACTCTTTCCTCCTGTCTAACTGTAACTTTGTACTGGTTGATGAACCGATAATTATGCTTTTTAA
GCTCGTGGAAAATGTTTCAGATCTTTAACCACAGTTAGTAGGCAAAAGTACTCATACTCTTTCTTTGATT
TTTGTTGTTGTCGTTCTTGAGATCCTACTCAAATCAATGTGGTGAAGCCTTCTGAGTATTGCCAGACCAG
ATATAATTCTTGGGGTATGACAAGGAATGAGTAGTAGCACCTTATGAAGCCTTAGGTTAGGTATACATAG
AAATAATCATGCTGTTACTTGAGATTGCCCTGCCCCTAAATAAGTGGTCTTGAAGATGTGGTAGGCAAGG
GACAGAGAAAGACCAAGCTCCCTGTGACCAGAAAAGGATTATCTGTTATTTAGAGGAAAGGGTTAGAGAA
AAGCTGGCCCATTGAATTAATGCAAGAAACCAGAGGTACTGTTAGGATATTCTGCAGACTAAGTCAGGGT
GAAAACATATTTAAGAACAAATTCAGCAACTGAGAAACTTCATGTCTTGAATGTGAGAGGCAATCTCCCA
TATGAAACTGTTAGTTGACGTTGAAAATTCATTCATCCTGGTGGTTGGGTTAGGAGGAGACCCAAGAAGA
GCTTTTGTATTTAGTTTCCTGGTGGTTGGAGAGTTCAAACACTAGACTTGGAAGCAGATAATACCTCATA
GTACAAGGAACAGGAACTTTTCTCACTGTCTGGAAAAACCAAGCATCTGGCTGTCTGATTTTAGTGAAAT
TGTAATTCAGGGGGAACTGATTAATTTTATTAGACAGGGTGTGAGTCCTGTGGCTCCTGCCAGGTGTAAC
TGAGATCCACTCTTGCCTTCTCTCTCTGCTCCTGCTATTTATGATCCTTATGCTCAGAGTGTGTAGAGTA
GGTTTGCATTCTTTTTTTTCTTTTTAGAGACTGAGGTCTTGTGAAGTTGCCCAGGCTGGATTTGAACTCC
TGGGCTCAGGCCATCCTTCTGCCTTAGCCTCCTGAGCAGCTGGGATTACAGGCATGCACCACCTAGCTAG
GTTTGGGTTCTGTTTGGCTACACTTCCTCCTATTCCTGCCACCAAGGTTTGTAGTTGTCGTTGATTTTAA
TAGACTATTTTTTAGAGCAGTTTTAGGTTCACAGCAAAATTGAGCAGAAAGTACAGAGATTTCCTCTATA
CCCCATGCCCCCACATGCATAACATCCCCCTACTGTCAACATCCCCCACCAGAGTGGTACATTTGTTAAA
ATTGATGAACATGCATTGATACATCATTATCACCTGAAGTCCATAGTTAACATTAGAGTTTACTCTTGGT
GTTGTACATTCTATGGGTTTGGACAGATATACATGTGTCCGCTATTATAGTATCATACAGAATAGTTTTA
TGGCCCTACAAATCCTCCGTGTTCTACCTATTCACTCTTCCCTCTCCCCTGCCCGAGTGGAATTTCTGTT
TTTATTTTATTTTATTTTTTGTTTATTTATTTTTTTGAGATAGGTTCTTGCTCTGTTATCCAGGCAGGAG
TGTGGTGGCACAATCATAGCTCACTGCAACATCTACCTCCTAGGCTCAGGTGATCCTCCTGCCACAGCCT
CCTGAGTATCTGGGAGTACAGGTACACACAACCATGCCTGGCTAATTTATTTTTCTTTTTTTTTTTTTTT
TGTAGAGACAGGGTCTTGGTATGTTGCCTAGGCTGGTGTCAAACTCCTGGGTTCAAGCAATCCTCCTGCC
TCAGCCTTTGCAATCCCAAAGTGCTGGGATTACAAGCATGAGCCACCACGCCTGGCCTGTTTTTATTTTT
AAAAATTACTTGAGGAACTGGTTCTAATTCCAGTCCTGTGCTGAACTTGTATCATACATAGTTCAGAGAA
TTCCTACCTATGTAGGGACTGCAGATATCCTCCCCTTATCCCTTTCCTCCTTTCCCTTCCCCAAATAATG
GCAAGTCAGTGCCCATACTGTGTATCAGTCCATTTCATTTTTTGAGATTTCCTTGATGGTCTGCTAATTT
GGAGACACTTCTGGACAAATGATAAATTTCTGGCAGTACAAGCCCAAAGTAGTAAGGTTTTGTTCTAGAC
ACTGGAGCTAGAATGAAATTTAAAAATGGTTCCTTGCTTTTACACTGTTGGTGGGAGTGTAAATTAGTTC
AACCATTGTGGAAGACAGTGTGGAAATTCCTGAAGGATCTAGAACCAGAAATACCATTTGACCCAGCAAT
CCCATTACTGGGTATATACCCAAAGGATTATAAATCATTCTACTATAAAGACACATGCAAATGTATGTTT
ATTGCAGCACTGTTCACAATAGCAAAGACTTGGAACCAACCCAAATGCCCATCCAATGATAGACTGGATA
AAGAAAATGTGGCACATATACACCATGGAATACTATATAGCCATAAAAAAGGGTGAGTTCATGTCCTTTG
CAGGGACATGGATAAAGCTGGAAACGATCATTCTCAGCAAACTAATACAGGAACAGAAAACCAAACACCA
CATGTTCTCACTCGTAAATGGGAGTTGAACAGTGAGAACACATGGACACAGGGAGGGGAACATCACACAC
CAGGGTCTGTCAGGGGGTGAGGGATTTGGGGAGGGACATCATTAGAAGAAATACCTAACGTAGATGACGG
GTTGATCGGTGCAGCAAACCACCATGGCGTGTGTATACCTGTGTAACAAATCTGCACGTTCTGCACATGT
ATCTCAGAACTTAAAGTATAATAAAAAAATTAAAAAATGGTTCCTACTCTCTATTTACAAATTATGACCA
CTTCATCCAAAGATCACAAATTCTAAAGTGATACAATCTTTCTGGGTAGCCTTAACTAAAAGAGCTGTAT
CACTCTTGAGTTCCTCTATGCATATGCCAATCATTTCCTCTTTGCTACACCCTGTTTTGAAGTGTCTGCC
CAGACAGTAGTACTCATCAACCACCAATCTTTGTGGGATGAGCAACTGACCCCGTAGAAAACTATCTAAA
GGGTCTGTTGGCTGGATTGAGCCAATCACATCCTCTTCCCTGAGAATGGGGACCTTGAGATACTGAGCTG
ATTAGATAATGGTTAGTTCTTGGGCTGAAATTTCGTATAGAATTGTGACCACCTTCTGAACTACAGCATA
AAAGACGTGCAAGCTGGGGGAACAAAAAAAGCCACAGACAGCAGAGACAGCAGAGGCAGCTAGTGTACAG
AGAGATGTGCTTGAACGGAGCAGAAATGTGGACACCCTGAGATGTGGCAGGAGCTTCTAGTTCCACTTCC
TATTTGGACCAGCTAAACCTTTTCTTTCCTTGGGTGTTGTTCATGAGATCTTTGGTCATAGCCTTTTCAT
AACACCTCTTCCCCCATTTGACTTGTGCTAGTTGAGTGGATTTCTGTTCTAGAGCTGAAATGCAGTGCCA
GCAGAATCTAGTGCAATAGAGTCTTATGTAATTAAAGTCCTAAAGACTCTTTGGGTTGTGAGACACACAA
AAAGATAGAAAACCAAGGTTGATAATCAGTACAAAATAACTACATGTTGGGGTACCCTTCAGTCAGTCAA
TGTCTTCCTCACCGGTAGGCATTTAGTGACTCGTTCACCACCCTTCCCTCGTCGAGGCTAAAGAATGCCT
ACGTCATTTCATCTTTTCTCAAAAAAGAAAAACATTCTGCTGTTTTCTGCTTCTTTATTGATCACATTTG
TCCTTAATTCCTTCATATAACCTCAGGTAGAATATTAGTCTAGGTGCCAGGGGATGTGGGTTCTGTTCTT
GTTGGGGTTCTAACGGGATGGATGGTCTCTGTAAGGTTCTTCATCTCTCTGTACCCCTACCCCCACACCT
TTGCCGGACAGTGAAGAGTTATGCTGATCACCGATTTCTAGCTGATAACTCCCACAAAACAAGGTGTGAA
TAAAGAGTGTGAAGAAATCAAGCTACTTCACAGGCATTTGCTATAGATGAAATAAATATGCCTATCCCTC
ACTTAATGATGTTAATTCTAAGAATCCTGCCAGTAAGTGAAAAGCTAGAAAATGAAAAATTACTTCTGTT
AATTTTTTTGTTGTTGTTTTGGTTTTGTTTTTTGAGACAGAGTCTTACTCTGTTACCCAGGTTGGAGGGC
AGTGGCGTGATCTCCCAGCTCACTGCAACCTCCACGTCTCGGGTTCAAGCAATTCTCGCCACAGCCTCCC
GAGTAGCTGGGATTACAGACATATGACACCACGCCCAACTAATTTTTTCTATTTTTAGTAGTGACAGGGT
TTCACTGTGTTGGCCAGGCTGGGCTCGAGTTCCTGGCCTCAAGTGATCCACCCGCTTGGCCTCTCAACTG
CAGCCGGCTAACTTATGTTCATTTTAGTGGTAAAAGTTATGATGTGTTCCATCCCAACCTAAGATGTCCA
AATACTTTCTACAAATAGAAAAATATATCATTGAGCAATGAAAACTTTTTATTAGTAAATAATCCAAAAT
TAAATAAATGTAATTGGTACATAAAATGTGTTAAAATGATGCTTTGTTGCCTCAATAAAAAAGAGGAGGC
GGGGCCGGGCGCGGTGGCTTGTGACTGTAATCCCAGAACTTTGGGAGGCCAAGGCAGGCGAATCACGAGG
TCAGGAGATCGAGACCATCCTGGCTAACATGGTGAAACCCTGTCTCTACTAAAAATACAAAAAAATTTAG
CCAGGCGTGGTGGCGGGTGCCTGTAGTCCCAGCTACTTGGGAAGCTGAAGCAGGAGAATGGCGTGAACCC
GGGAGGTTGAGCTTGCAGTGAGCCAGGATCGCGCCACTGCACTCCAGCCTGGGCGACAGAGCGAGACTCC
ATCTCAAAAAAAAAAAAAAAAAAAAAAAAAAAGAAGAGGCTTGATGGGATTGATGGAAGAGGGAAGGAGC
CATACTTTGGAAGGTGGTTGGTGAGTCAGGTGATAAACTTTTAAGGCGTGCATTCAGCAGCAAAAGTGCT
TAATAATATTTTTCATTGGGGATTTTGACAGTAGAAGAAACTCCTAATGTGGAAACCTTTGGTATATTGG
TAGAAGGGAATCTTCAGATCAAAAAAAAAAGCAAGATGTAACTTTGCTTCCCTTTTTGTAATTTGCAATT
CATACACCTCCTTGCAGGATGCGATATTAGTTTCCCGAAGCTGTTATAGCAAATTACTACAAACTTGTTG
GATCAAGAGAACAGAATCATATCTTGTTGCAGTTCTGGAGGCCAAAAGTGTGAAAACTGACATGGCCACA
TTCCCTCTGGGGACACCTTCCTCTCACTTCTGGCGGCCAGCATGGCATTCCTGGGCTTGTGGCCGCATCC
CTCCAATCTTTGCTTCTGTCTTCACTTTGCTGCCTCCTCTGTGTCTGTCTCCAACTTGCTCTGCCTCTGT
CATAAGGTTGCATGTGGCCTACCCTACCCTGATATTCCAGGACAAGCTCCTTCTCTCAGGATCCTGAATG
TTACCACTTTGTAGTAGTTGTTGTTGTACAAGGTAATAGTCATTCCTTTGCCATGTAAAGTAATATTCAC
AGGTCCCATGTGCTAGTGTGGGAATGAGGTGCCGACATTTGGGCTGCTAATTCTTTGTTGTGGGAGACTG
TCCTGTGCTTGGTAGATGTTCAGCGGTGTACCTGGCCTCTGCCCACTGGGTGCCAGTGACATCCTTCTCC
CAGCTGTGACAACCAAAAATGTCTTCAAACATTGTCAAGGGTCCCCTCTGGGGCACGACTGTCCTTGGTT
GATGAGCACTGCTTTAACCTAATCAGAGCCCATTCTTATCCCACTCAAATATGCTGACTGGGAAGTTTCC
AGTCTCGTTAAGAAGGGGGAGGGTGGAGAATGGATGCTGGAGAGGTGACTGCTCCTCTCCACTACACCTA
ACATTCTAGTGAAGTCCACAAATGGGCCAAATCGAGCAGGATTATGAAATTTTTGCTAGACCCCTGTTAG
TGTATTCTAACATTGTGGCATTTTATATTAGTCAACTAAATCTCAATATTATGTTTTAAAAAAATCCAAT
GGACCTCTGAATCCCCAGGTTGGGTTTTTTCTACTTTACCTATGTTAAATATGTTAAATACCTAGGTTGC
CCTTTATCTTAGAATTTGTCCATGTATCACCTCATTCTTTTTCAGCAGGTCCCTTTCTGGAGGTTTTCAT
ACTCAGTTGGATTTATCCTCCCTGCGACTGCTGCCTGCTCCTTGCCTTGCCATGCGGCATTAGTAGAGTA
TGAACTGCACATTTAGCACAGCAGTTTATGCACTAGAGAATCAGGATTGGAATTTTTCTGAAGTATCTTA
TTTTTCTGTGAAATAATTGAAACAGAGTATAGACACATTTGCTGTTTTGAAGTTTTTATGATTTCCAAAG
TAATAAATGTGCACTCTCACAGTTCTCGGGTGGCTGATCTCTGGAGTGCTTGCCGTTTCTCCGGGTGGTT
GCTTTGTACAGGCTCCCCTCTCGTGTTGGCCTGCAAGTGCTCTCGCTGCGACGGGTCCCTGACCGGTACT
GGATGAGAGGACTAGTACTGGTGCAGATGTTGGCATAAACAAAAGCTGCAGCTTTTTAATCATGACTAAG
GCATGGGAGAGGCTGACATATAATTGTGGGGAAAAGCGAGAGAGATCAGATTGTTACTGTGTCTGTGTAG
AAAGAAGTAGACATAGGAGACTCCATTTTGTTATGTACTAAGAAAAATTCTTCTGCCTTGAGATTCTGTT
AATCTATAACCTTACCCCCAACCCCGTGCTCTCTGAAACATGTGCTGTGTCAACTCAGAGTGGAATGGAT
TAAGGGCGGTGCAGGATGTGCTTTGTTAAACAGATGCTTGAAGGCAGATGATCAATAAATACTAAGGGAA
CTCAGAGGCTGGCGGGATCCTCCATATGCTGAACGCTGGTTCCCCGGGTCCCCTTATTTCTTTCTCTATA
CTTTGTCTCTGTGTCTTTTTCTTTTCCAAATCTCTCGTCCCACCTTACGAGAAACACCCACAGGTGTGTA
GGGGCAACCCACCCCTACAATAATGGTTCCCAGAGTGTCCCTCTCAGACCAATAAAACATAGCAAAACTA
CGTGATAACCAGGCTGGAATGTATGTGAATTGTTCGAGTGAGTCTGTGTCACTTGAACTGTACTGGGCAG
TACCTAGGGGCTTCATGTAAGGCATGAAGGGCTCCAGAAAGAACCCGCTCTTTGCCTTCAGTTGTATTCT
ATATGTTTAATACACAGCACAGCAGAAGCAATCAGCTCTCCCCAATGTTACTTATCCCATAAATCATCTT
TCCTTACACCTACGTATTTTCTTCTGTATTCAATTTTATTAATGGATCATCTGATCTCCAGCTTTGCTGG
TTTAGAACCTCTGTGCTCATCTTAAGCTCCTCAAGGAGAAAAGGACTATTGTAATCATCTTTCAACGAGT
TTGTAAAAACAATTCCCCCATCACAGCACTGTGCCTCATAGGGTTTAAAATTTGACCGAATGACTGACTT
TACATACATCCTTCCCAGACCCTTTAGGACTACTGCTCCCCTAGAGGTTTTCTCTGCCCTTTGGAAAACA
GGTAGGGGAAGAGGGATCTAGAAATGGCAGTATTCATTAAGATAGCCAAAGCTTGATAGGAAGAGACATT
CATTTTACTGTGGTAATTAAAGTGAAAATGGGAGAATAAGAAGGCATGCTGCTTCTTTTTCAGGAGTCTT
GGCAAGGGTTCTAGTTTTAAAAGAAAGAGACACAGCCAACTAGATTGAAATGATGTGGAGTTCATCCCTC
CACAGTCATATTTTACTGAGCCATTTTTCAATAAATGGACTAAATTGTCCCTTGGAACTCAGTAGGTGGT
GCTCTTGTGCTATAGCATAGCATCGGCTTTGGAGAGGTTCTGAGAGCACAGCGGCTGGTTCCAGGGCCTG
TGTCCTGTCAGTCCCCTTTCATAGCTGCCAGGCACACACCTGGAGGGCATCACATGGCTGGTCTGGTGAG
AGGCTTTCTGTCTTTGGAAATGAGGGGGTGAATTAAGGTCTTTACCTTCTCTAGCCTGTCTGAGCTCTGC
TTTCCAGGGGAACCTAAATGAGAGGTTTTCTAACCTTTTAAAGAAAAAAAAAATGTGACTGTTTATCCAA
ACAATTACGATGTGTATTGTTACCAGTTTTCCAAGATTTTTTATATTGTTATTCATCTGTGATTCCTCAA
ATCAAAGTTCCTTTCCACATAACTCAGGGTATCATTCTTGTTTATGTTTTCCTTTATGTTAATGTTTGTT
TCCTGTTTAAGCTTCAGAGAAGATGCAGCAGAAAGAAATAAATATGTAGTAGAGAAATTTGGAAGTGATG
AAGGAATACAGGTGAAAGCCATCGAGTTCATGCAGCAGCTAGTAGTAACTTAGCTGACAACAGACCCTTT
TGGCAGGAGCCCATTGCTTTACTCCTTTTGCTATATAATGAGTTCCTTGGTCCGAAGTTGGTTGTGATAC
CATGATGGTGAATAAGGAATCCAGTCAAGTCCACATTTGGTGATGCTGGAGAAGTGTGGTGGGCAAAGAA
GATAAATTTAAATCCAGAGTGAGTTATCTATTACAAGGAGGACAAATCTGTTGCCTGAAGCATTTCAGGG
TCTGCTTTTTCATCAAGTTTCTGTAGGGGTTGTATAAAACGAAAGTTACTTCACCTATATCCACCAACTG
GTAGGGGAGAGGCAAAATGCTAGTGGGACTCACAGCCTGGATGTGCTGATCTGACCCATTCAGTCACCTG
TGATGTCGAAAGGCTACCAGCCAGGTCCGAGTGAGGCCAGGCTGTGGGGCCAGCAGCTGGGCTCCTGAGC
TGTGCTGCTGCAGATCCAGTGATGTGCAGTGTGTTTTGTCAAGACAGAGTGCTTCCTCTCCAGTCTACTG
TCTAGTATCTTGTGCACTGAGTTTTTAGGTTTTAATGACCGTAGTCCTGATTTACATACTTTGCAGTTGA
TTTTTTTTTAGCCATCTTTTAATTCTGCCTTTTCAATTTATACTTTCTTATTTTTTAATTAGCTTTTTTA
AAAAGCACCCTAAATATACTGATTTTTGTTGGTTTTAAAAATCACCTTAAATATATTTTGTTGGGATTAT
TTTAGTGTCTTAAGTATACTTTGTTAGGATTTTTTAGCATCTAAATATACCTATTTGTTGGACTGATTAA
CTTCATCTGAAGTGAATTTGTGATCTAATGTCTGATATTGTTAGTTGCATTTATTTCTTCATGTACTTTA
GAACTTTGGTTAAATGACATGTTTTGAATGAGGTTTTAAAAACTTTTCACTCATTCAGTCATTTTTCGCC
TCTTTCTCCCTCTCTGCACACTCTCCCCTGTGTAGCCTCATTCTGCTTGTCTCTGTCTGGCTTCCATGGA
GCCTGGGTGCAGAACTAGCTTTCACAGTGGGGGCTTAGTTACCTGGGCATTCATGGCTGCTTCACGCAGT
GCCTGGTCCTGCCTGACAGGTGTCCTCCATCCTCCCTAGACCGCCACTCCATAGGAGGCAAGGATGCTGC
GAGCCTTCCTTCAGGCATGGAGGGGTAGGGCTCTAGTCAGCACCTTCCATCCCACCTTCCAGCAAGGGTG
CTAGTTTCTCACTCTGTCGTTTGTGCCTCTGTTTAGCCCCCAACCCTTCTAAGACCCAAACCTACAGTTT
CATTGCCTGTTTTTGGACCCTAGCATAGGAGGGGCTGGCTGCTCATGGTTTTGTCCTCGGGGTCTTGCCT
CTTTGTGTTTCTGTCTCGTATCGATTTCATAGAAATGTTTATTTTGTGTGTGTGCTTGCCTGTATCCTTT
TCCTTTTTTCTTTTTATGTTTCCCCTCTCATTGCTGTATGTTTGGAGTAAAGAGAATCCATCCAAAAGTG
AATTTACCGTGCCATCTTAACTGGAAGTCACATGGGCAGAGAGTTTAAAGGAAAGAAAGATACTTGGTTT
TACATAGTACTGAAAGCTAACCACAAGAACGCTTCCTATGTGAGCTCAGTGACCCATAGCATATAGTTAC
TTAAACTCCGTCTGAGGCTTGCAGGCCCCAAAAGATGAGTGGGATTAGGCAGGTGGGATTTATTAGGGTG
CTATGGTTCTCTACAAAGGTTTCACCTTCTATTTTTCATTTTGGTGGTAATTTTTTAAAAATTCATAATT
CCACACACCTAAATGCCTGCAATTCTAACTAAGTAATGTCTTAAAATTTCAGTGCCTGGAGTTGTGTTTG
TATTGGTGCCAAGGAGCACTGCTTTAAATATAGGGAGATATTATAAAAGAACAGAGGTGAAAATGTAAAT
AACGTGTTTGAGCTAAGAGATTGCCGTTGCACAGTGGTTCTGAAGAGGAAAGTGATGCGTGAATTAAGCT
GTCTCCCTGCATGTGTTAGTATTGGCCAGTCAACAGAACCTGCCTGTCGGTACCAAGCCCTACATGGTTA
GTCCAAAGTGAGTTTCAATGACTGCATCATTGATCACGCTGAATTAATTTTGTCAATTTAAATTTTACTC
ATTTTGTAGATTTGTTGGTATTTAATTTGTATATTTTTTATATTGTGGTTGTGTAAGAGTTACAAGCATA
AAGGTTTTACTGGTTTATGTTTGTTAATATTTAACTAATAAGAAGAATGACTTTAGATAATACTGGGAGC
TCTCAACAGTTTTTAAGTTTGAGAACCATGGGTCAAATGGTAATTTAACTCTTGCTGTTGGTTGCAGCAA
GCACCTTGCATTTGATTTTGATGTCTTTAGCTCACTCCTGCTCATGCTTCCTGACCTGCTTAGCTCAGGA
TCGTTTCTCTCTGGGGACTCTGACACCTCCATGCCGGGCTGGTGCCCCTTCCTCATGCTGCCTGCAGCCC
CCTGTGTTTCCTTCTCTCCCAGCCCTTCCCACAGTGTAATCTGTGTATGGGTCCCAATTTCCTCATTAGC
CATAAGTTGCTTGAGAGTGGGGAACACTGTCATCTTTTTATTTGCAGTGATTTTCAGAGCTCTTGTCATA
AAATAGGCTCCTAATAAATGTATGGGTTTCCTTGTTTTTGTTTAGGTAGTAATAAATTACCTGTAGAATC
AAGGAGAGAAAGAGGCAGAAATAACTTGGAGCCAAGTCACAAGGCAAGAAGAAGCTTCCTAATATTTTAA
TAACTAATGTGTTCCTCAGCTCACTTTCTAACTGGTCTAACCAAAACCCAAGTCTTACTGTCAGGTAAGT
GAAGTGAGGACAAGAAACGCAAGGAAAGGAAGTAATGATTGTAATAGAATGCATGAGGGAAGGCTGGTAT
TTTTCAAGTCCGAATGATGGCTCCTTGGTGCTCTGATGGAGAACAAGGTCAGAGATTTCTGTCTTAGCAG
CCTCGATGGCAAGCGCTGTGGTGAAGGGATGCTTTTATTTTGAAGCCAAGTCCAGAGAATAGCTCCTCTC
CCCCCATTGCTGAGGTCTTGGGTGGGAGAATTGAGCCTAGTAAATCCCCAGAGTTATCTGCTAGATTCAG
TTTCCTTAATTCTTTGCAAAGTCATCTGAAGAGTATATATACATACTCTGCACCTGGATTGAAGTTGCCT
CCTCCAATTCCTTAGGCCCAGATTTTGTTTACAATTGAGGTTTGTTTGAAATTATCTGCAAATTTCAGGT
CTGTGTTTTTGATTAGACTATCTCAGAGGTGGAACGCCACTGACTCAACAGAGAAATAGCCAGCTCCTCC
TAGTGGAACTTTTCTTAAAACCTCATTGGTTTTAGGAGGAAAAATACCATTTGTTTCTTTCATAAGCACT
TAATGGGAACAAGAATCTTTTATTGTGACTTTCAAAAAAAAAAAAAAAGCCACAAACACAGGGGTAAATT
GACCAGTCTTACCCTGTGAAGTGAAGCAAAGAAACATTACATTTCAGCAAATTAAAAATAGTTATCAGGC
TTTCATCAAGGAAGAGGAAGAATCTTGTCAACTTAAGTGGAATTGAACATAAATTTCTTAATTTTATATT
TGATTATATTTTAGGGGGTTTTAAATATTTTCTGACAAGGTCATGGTGCCCCTAGTTGATGACATTGATT
GATTGGAAGCTAAGTATTTGATTTGTCTTTTTTTTCTTTTTCCATAATTAGTGTGTTTTGTAGCATTCTC
TCCATACATCTCCTGTTTGGTGCTTATTTCATGAGAGAAAGGACCTCATGCTGAGGGTAAATGGGAAAAA
AGTGCACTTTATCAGGTCGCCTCCACTTTGAGAAGCTTATATGGTCCATATTTGGCATGAGTAGACTTAG
CTCTCTGGGCTTAGAGATGTTTTAGACCAAAGGAAAGCATTCTTAGACTTTCTTGTCCCCCTGACAGGTA
CCATGTATAGCTACTTTTGTGTGGTTTAAAATGAGCTTTGTTTTAAATGTTAACAATCTTGGAGCTACAG
GGGTTTCTATCAACATAAAATCAGTCAGAATTTTATCTGTTTTATTTTTCTTCTACAACAGCCTTGTTGC
TTTTTTTATGTATTTATTTTCCATACATTTGTTCAGTACATATTTATCATGCTCCTACAATGTGCCAAGT
ATTGTGTGAGTTTGTGGGTTTCAAAATATGCTCAGCAAACTAGGTCCTGAGATGTGTCAGCTAGGATGTG
TTTGATTGCAAGTAACAGGAAAGCCAAAGAATGGTGTTAAGGGATTAAGCAAAAAGAGCAGTTCCACATT
TGGCTCCTTCATTGCCTCAACTATGCTGTGAGGACACGGGGCTGTGATTCTTCTTTGCTGTTTGCAGTAT
GTTGGCCAGTCCCCTCAGGTTTCCTTATCCCTGGGTCTCAAGGTGACTGTTACCGTTCTGAGTATCACAC
AAAGACTACTATGTCAGATATGGAAAAAAGTGAACTCATTTAAAACTGAGGAAAACTGAGAAACCTTTTG
TTGAAGTTCAGTTCTCATGCCCTATATTTCCATAATATACCTGTTCCTACAGTTTGATAGGCGAGCAAAA
GTAAATACCATGACTGGTCTGTACTAGCCATTCCCAAAATGTGGCTGAGGGCCCAGGGTCCCCGAGACCC
TTTTAGGAGGTTCACAAGGGTAAAACTATTTTTATAATAATGCTAAGCTGCTATTTGCCTTTTCGACTCG
CATTCGCTCACTAGTGTGCGGTGGCGTTGTCAAGAGGTTACATGATAGATGATGATGACCTCTCTCTGAC
AGCTAATGGAATGTGTGTTTGTATATTTTGTCTTTTTAATTGTTCTCAGTTTTAGTTTCTGGTGGTAAAT
ATCTATCTATCTATCTATCTATCTATATCTATATATATATAGATATATATATATATACACATACATACAC
ACACACACACATATATATATAAATAAAACTCTTTGGGGTCTTCAGTAACATTGAAGGATGTAAATGGGCC
CTGAGAACAAAACATTTGAAATTACCTGCTCTGTGTGAATCAAGATTCACCCCTGGCACTGGCGGATATC
CCAGTCTCCCTAAGACATATGGCCTTGAGATTCCTGGACAAAACTCTTAACAAAGAGGAAATGGAGAGCT
TAGGGCTGCTGGGTGGGCAACTAGCAGTGTGTGCAGCCCTGTTCTGAGGGGGCCCTGAGGAGACAGCAGG
GCAGGGATGCAGCATGTTCTCCAGGCCCCACCTCTGCTGGGCCAGCCAGGACTGGAGCCACTGTAGGTTT
GTCATTTGTGTGTTGTTGGTTTTTTAATTGTGGTAAATATAAATAAGATTGTAACCATCTTAACCACTTT
TACATGTAATTCAGTGGCATTGATTACATTAACATTGTTTAACCATTGCCGCCATCCATCCCCAGAACTT
TCCCGTTATCCCAAACAGAAACTCTACCCATTAAGCAGTAACATTCCATTACTGTCTCCCCTCATGAATT
AATTTGCCTCTGTGAATTTGACTATTGTAGGTACCTCATCTGTGTGGAATCAAAGAATATCTGTCCTTCC
GCATTTTGTCTGATTTCACTTAGCATGGTGTTTTCAAGATTTATTCCTGTATGTATCAGAATTACATTCC
TTTTTAAGGCTGAATGATATTCCAGTGTGTGCATATGCCACACTTCGTGTATCCTTTTCTCCACTGATGG
ATATTTGAGTTGTTCTCACTTTTGGCTGTTACGCTGCTATGAAATTGGGTGTACAAATATCTCTTTGAAA
CCCTGCTTTTAATTCTTTTGGGTATATACCTAGAAGTGGGATTGCTGGATCAATGGTAATTCTATGTTTA
GTTTTTTCAGGAACCACCATACTGTTTTTCGTAGCAGCTGCGCCATTTTACATTCCTACCAGCAGTGCAC
GAGAGTTCTGGTTTCTCTGTGTCCTCATCAACACTTGTCGTTGTTTTCTTGATAGTAGCCGTTCTAAGTG
AAGGGAGATGGTTTTCTGTTTGTTTTTAATGAATTAGGATGCATTGCCCCAGCCTGTCACCCAGCGTCTC
CTCCCTCTTTGTTATCTTTCTTCAGACACTGCTCAAGTGTTTTAATGCTTTTACTTCCTTCTGCTCTCAT
GAATTGTGCTTGGTGAGCTCCATATTCTTGTAAGGTACATTTTCCATGAAGTTTTTGAATATATCCTTCT
CCTAAAGGATAGAATTCACCAAAATGAGGAACCTTTAGTCCTGAGGTAATACTCTTTGTTGCCTGCTGTA
CTTTGAATCTGAGCCATGTTATCTCGGTGACTCAGACCATGAGCCCCTGTGGACAGTGGTGTATCTTTGT
ATCATGGCTATTTGAGTTCATAAAATAGTACTTAACAAGCAACCAAACTCTTGAGCAATTTATATGTGTG
AGAAGAAGGGCAATGGCAGTGGTCCTAAGCTGATGCCATTGTGAACTGGTTCCTTTTCAGTATCTTGGAT
GTTTGCATGGCCCCATGGCTGGTTTCCCTTGGTACTTTATCCCAGCCCCTCACCTCTTCCTGCTTCTGAA
GGATCCGCTCCCACCTGGCAGGAAACAGGGACAGGATCAAGCATGTGTGGGTCCTTCACTGGACACTGCC
TTCTGTTCCAGCGTCTGCATCTCTCTGCTCATACGACTTTGCTTTAGTCCCCTCTGCATAGGAATTTCTG
AAGGTCTTTATTTAATTTCTTTATTTTCTCCTGGAAATACTTACTGAGGGATCTAACAGCTTCTGTTGTT
TTTAGTCTGCATACGTGGAGAAGGTGAAATCATGGTTTGTTTTGATTCCTTAGGGACTGGGTAAATGCAC
TAAAAGATACATTATGCAAGAAATGGAAAAAGTGGATTTCCTTCTGATTTTTCATGCCAATTGCTTTTGC
TGCATTAGTATATGCTGTGCGTGTATGCACAAAATACTTGTTAACATCCTGAGTTTTCAGGAAATATGTT
ATAAAACATTTACCTTTTTTATTTCTTAAAAATGCTAAACAGTATTTCTCTAAGAGCTAGTAATGAAATG
AATTTAGGTACATACACTAGGCAGAATCATAAGAAAGTCAAGGACTTACATTTGGAGCAAGATAAAGGAT
TTTGGTATTGAGCAAAAAATATAGTCAGCTGTCAATTGTCTAATAGGTTTGTAGATTACCTGCGATAAAA
AAGTAGTACATCTGTGAAAAAAGTTGAAATTACCATAAATCTGAAGAATCAGCCCCATTTTTCAAGGCAT
GCATTTTTTGGAGCTGCACGGAAGCCACCTTATATGTCTGCTTCGGGTGACACTGTCTTATTCCTCCGTG
AATAATGAATTGAACGTCTCTCCCTCTTTGTGACAAAGCCCTCTGGGGCAAAGCAGTAGCCGTACAGGTA
AGATAGGGGGTCCAGACAGCAGGTTTTGGATTTTCCCCTCCTTAGAGCCAGGACTGGAAAAATGGAGTCA
TTATTTGAGGACTGGAAGGAAAAGGAGGGAGCAAGTGCTAGGACTGTTATCTCTGCCCATCCTGGTGGGT
GGTTTTAATATTTCCAGACAGAGAACTCAGTTAAAAAAACAAACAAACAGAAAATACATACTATTTCAAC
AATTTGCAAAATTTTGTTGTATTCATCAAGTACTGTCCATCTTTATTCACATTTATAAACTATAATACTT
TTTCCTTTTTTCTTTGCCTGTGGGGTGATGTCTTTATGGTAAATGTGTATCTGGAAGTTTTTTTTTTTTT
TTTATATTTTAAGTTCTGAGATACATGTGCAGAATGTGCAGGTTTGTTAGCCAGGTATACACGTGCCATG
GTGGTTTCCTGCACCCATCAACCTGTCATCTACATTAGGTATTTCTGCTAATGGTATCCCTTTCCTAGTC
CCCCAACCCCCTGACAGGCCCCGGTGTGTGATGTTCCCCTCCCTGGGTCCATGTGTTCGCATTGTTCGAC
TCCCACTTATGAGTGAGAACATGTGGTGTTTGGTTTTCTGTTCCTGTGTTGGTTGCTGAGAATGACGGCT
TCCGGCTTCATCCACGTCCCTGCAAAGGACATGAACTTAGCCTTTTTTATGGCTGCATAGCATTCCATGG
TGTATATGTGCCACATTTTCTTTATCCAGTCTATCATTTATGGGCATTTGGGTTGGTTCCAAGTCTTTGC
TATTGTGAATAGTGCTGCAATAAACATACATGTGTGCATGTGTCTTTATATAGCAGGATGATTTATAATC
CTTATATACCCAGTAATGGGATTGCTGGGTCAAATGGTATTTCTGGTTCTAGATCCTTGAGGAATCTCCA
CACTGTCTTCCACAATGATTGAAGTAATTTACACTCCCACCAACAGGGTAAAAGCGTTCCTGTTTCTCCA
CATCCTCTTCGGCATCTGTTGTTTCCTGACTTTTTAATGATCGCCAATTCTAACTGGCGTGAGATGGTGT
CTCATTGTGGTTTTGATTTGCATTTCTCTAGTGACCAGTGATGATGAACTTGTTTTCATATGTTTGTTGG
CTGCATAAATGTCTTCTTTTGAGAAGTGTCTGTTCTTATCCTTTGCCCACTTTTTGATGGGGTTGTTTTT
TTCTCGTAAATTTGTTTTAAGTTCCTTGTAGATTCTGGATGTTAGCCCTTTGTCAGATGGATAGATTGCA
AAAATTTTCTCCCATTCTGTAGGTTGCCCGTTCACTCTAATGATAGTTTCTTTTGCTGTGCAGAAGTTCT
TTAGTTTAATTAGATCCCATTTGTCAATTTTAGCTTTTGTTGCCATTGCTTTTGGTATTTTAGTCATGAA
GTCTTTGTCCATGCCTATGTCCTAAATGGTACTGCCTAGGTTTTCTTCCAGGGTTTTTATGGTTTTAGGT
CTTACATTTAAGTCTTTAATGCATCTTGAGTTAATTTTTGTATAAGGTGTAAGGAAGGGGTCCTAATTCA
GTTTTCTGCATATGGCTAGCAGTTTTCCCAACACAATTTATTAAATAGGGGATCCTTTTCCCATTGCTTG
TTTTTGTCAGGTTTGTCAAAGATCAGATGGTTGTAGATGGGTGGCGTTATTTCTGAGGCCTCTGTTCTGT
TCCATTGGCCTATATCTCTGTTTTGGTACCAGTACCATGCTGTTTTGGTTCCTGTAGCCTTGTAGTATAT
AATTTGAAGTCAGGTAGTGCGATGTCTCCACCTTTGTTCTTTTTGCTTAGGATTGTCTTGGCTATATGGA
CTTTTTTTTGGTTCCATATGAAATTCAAAGTAGTTTTTTCTAATTCTGTGAAGAAAGTCAATGGTAGCTT
AATGGGGATAGCACTGAATCTATAAATTACTTTGGGCAGTATGACCATTTTCATGATATCTATTCTTCCT
ATCCGTGAGCATGGAATGTTTTTCCATTTGTTTGTGTCCTCTCTTATTTCCCTGAGCAATGGTTTGTAGT
TCTCCTTGAAGAGGTCCTTCACATCCTTTGTAAGTTGTATTCCTAGGTATTTTATTCTCTTTGTAGCAAT
TGTGAATGGGAGTTCACTCATGATTTGGCTCTCTGTTTGTCTGTTATTGGTGTATAGGAATGCTTGTGAT
TTTTGCACATTGATTTTGTATCCTGAGACTTTGCTGAAGCTGCTTATCAGCTTAAGGAAATTTTGGGCTG
AGACAGTGGGGTTTTCTAAATATACAATCATGTCATCTGTAAACAGAGACAATTTGACTTCCTGTCTTCC
TATTTGAATATGCTTTATTTCTTTATCTTGCCTGATTGCCCTGGCCAGAATTTCCGATACTATGTTGAAC
AGGAGTGGTGAGAGAGGGCATCTTTGACTTGTGCCAGTTTTCAAAGGAAATGCTTCCAGTTTTTGCCCAT
TTAGTATGATATTTGCTGTGGGTTTGTCATAAATAGCTCTTATTATTTTGAGATACGTTCCATCAATACC
TAGTTTATTGAGAGTTTTTAGCATGAAGGGATGTTGCATTTTATCGAAGGCCTTTTCTGCATCTATTGAG
ATAATCATGTGGTTTTTGTCATTGGTTCTGTTTATGTGACGGATTGCATTTATTGACTGGCATATGTTGA
ACCAGCCTTGCATCCCAGGGATGAAGCCGACTTGATTGTGGTGGATAAGCTTTTTGATGTGTTGCTGGAT
TTGGCTTGCCAGTACTTTATTGAGGATTTTCGCATTGATGTTCATCAGGGAATTGGCCTGAAATTTTCTT
TTTTTGTTGTGTCTCTGCCAGGTTTTGGTATCAAGATGATGCTGACCTCATAAAATGAGTTAGGGAAGAG
TCCCTCTTTTTCTATTGTTTGGAATAGTTTCAGAAGGAATGGTACCAGCTTCTCTTTGTACCTCTGGTAG
AATTCGGCTGTGAATCTGTCTGGTCCTGGGCTTTTTTTGGTTGGTAGGCTATTAATTACTGCCTCAATTT
CAGAACTTGTTATTGGTCTATTCAGGGATTCAACTTCTTCCTGGTTTAGTCTTGAGAGGGTGTAAGAGGG
TGTATGTGTCCAGGAATTTATCCATTTCTTCTAGATTTTCTAGTTTATTTGTGTAGAGGTGTTTATAGTA
TTCTCTGATGGTAGCTTGTATTTCTGTGGGATCAGTGGTGATATTCTTTATCATTTTTTATTGTGTCTAT
TTGATTCTTCTCTCTTTTCTTCTTTATTAGTCTGACTAGCGGTCTATCTATTTTGTTAATCTTTTCCAAA
AACCAGCTCCTGGATTCATTGATTTTTTTGAAGGGTTTTTTGTGTCTCTGTCTCCTTCAGTTCTGCTCTG
ATCTTAGTTATTTCTTGTTTTCTGCTAGCTTTTGAATGTGTTTGCTCTTGCTTCTCTAGTTCTTTTAATT
GTGATGTTAGGATGTCAATTTAGATCTTTCCTGCTTTCTCTTGTGGGCATTTAGTGCTATAAATTTCCCT
CTAAATACTGTTTTCGCTGTGTCCCAGAGATTCTGGTACGTTGTGTCTTTGTTCTCATTGGTTTCAAATA
ACTTCTTTATTTCTGCCTTCATTTCATTATTTACCCAGTAGTCATTCAGGAGCAGGTTGTTCAGTTTCCA
TGTAGTTGTGCAGTTTTGAGTGAGTTTCTTAATCCTGAGTTCTAATTTGATTGCACTGTGATCTGAGAGA
CTGTTATGATTTCCGTTCTTTTGCATTTGCTGAGGAGTGTTTTACTTCCAATTATGTGGTCAGTTTTAAC
AGAAGTGTGATGTGGTTCTGAGAAGAATGTATATTCTGTTGATTTGGGGTGGAGAGTTCTGTAGATGTCT
ATTAGGTCCACTTGGTCCAGAGCTGAGTTCAAATCCTGAATATCCTTGTTAATTTTCTGTCTCGTTGATC
TGTCTCATATGACAGTGGAGTGTTAAAGTGTCCCACTGTTATTGTGCGGGAGTCTGAGTCTCTTTGTAGG
CCTCTAAGAACTTGCTTTATGAATCTGGGTGCTCCTGTATTGGGTGCATATATATTTAGGATAGTAAGCT
CTTCTTGTTGTATTCATCCCTTTACCATTATGTAATGCCCTTCTTTGTCTTTTTTGATCTTTGTTGGTTT
AAAGTCTGTTTCATCAGAGACTAGCATTGCAACCCCTGCTTTTTTTTTTCTTTCCATTTGCTTGGTAAAT
ATTCCTCCATCCATTTATTTTGAGCCTATGTCTGTCTTTGCACGTGAGATGGGTCTCCTTACAGCACACT
GATGGGTCTTGACTTTTTATTGAATTTGCAAGTTTGTGTCCTTTAATTGGGGCATTTAGCCCGTTTACAT
TTACGGTTAATATTTTTATGTGTGAATTTGATCCTATCATTGTGATGCTAGCTGGTTATTTTGCCCATTA
GTTGATGGAGTTTCTTCACAGAGTCGATAGTCTTTACAATTTGGTATTTTTTTGCGGTGGCTGGTACTGG
GTTTTCCTTTCCATATTTAATACTTCCTTCAAGAGCTCTTGTAAGGCAGGCCTGGTGGTGACAGAATCTC
TCAGCATTTGCCTGTCTGCAAAGGATTTTATTTCTCCTTCGCTTATGAAACTTAGTTTGGCTGGATATGA
AATTCTGGGTTGAAAATTCTTTAAGAATATTGAATATTGGCCTCCACTTTCTTCTGGCTTGTAGAGTTTC
TGCAGAGAGATCCGCTGTTAGTCTGATGGACTTCCCTTTGTGGGTAACCCGACCTTTCTCTCTGACTGCC
CTTAACATTTTTTCCTTCATTTCAACTTTGGTGAATCTGACAATTACATGTCTTGGGGTTGCTCTTCTCG
AGGAGTATCTTTGTGGCATTCTCTGTATTTCCTGAATTTGAATGTTGGCCTGTCTTGGTAGGTTAGGGAA
GTTCTCCTGGATGATACCCTGAAGAGTGTTTTCCAACTTGGTTCCATTCTCCTTGTCACTTTCAGGTACA
CCACACACCAATTAAACGTAGGTTTTGTCTTTTCACATAGTCCCGAATTTCTTGGAGGCTTAGTTTGTTC
CTTTTCATTTTTTTTTTCTCTAACCTTCTCTTCATGCTTTATGTCATTAAGCTGATCTTTAGTCTCTGAT
ATCCTTTCTTCTGCTTGATCGATTCAACTATTGATACTTGTGTATGCTTCACGAAGTTCTCATGACGTGT
TTTTCAGCTCAATCAGGTCATTTATATTATTCTAGTTAGCAGTTCCTCTAACCTTTTTTCAAGATTCTTA
GCTTCCTTGCATTGGGTTAAAAAATGCTCCTTTAGCTTGGAGGAGTTTGTTATTACCCACCTTCTGAAGC
CTACTTCTGTCAGTTTGTCAAACTAATTGTCCGTCCAGTTTTGTTCCCTTGCTGGAGAGGAGTTGTGATC
CTTTGCAGGAGAAGAGGCATTCTGGTTTTTGGAATTTTCAGCCTTTTTGTGCTGGTTTTTACTCATCTTC
ATGGATTTATCTACCTTTGGTCTTTGATGTTAGTGACCTTCGGATGGGGTTTTTATGTAGACGTCCTTTT
TGTTGATGTTGATGCTATTCCTTTCTGTTTGTTAGTTTTCCTTCTAACAGTCAGGCCCCTCTGCTGGAGT
TTGCTGGAGGTCCACTCCAGACGCTGTTTGCCTGGGTATCACCAGTGGAGGTTTCAGAACAGCAGATATT
GCTGCCTGATCCTTCCTCTGGAAGCTTCGTCTCAGAGGGGCACCCGCCAGTTGCCAGCCGGAGCTCTCCT
GTATGAGGTGTCTGTCGACCCCTGCTTGGAAGTGTCTCCTTGTCAGGAGGCATGCGGGTCAGGGACCCAC
TTGAGGAGGCAGTCTTTCCCTTAGCAGAGTTCAAATGCTGTGCTGGGAGATCTGCTGCTCTCTTCAGAGC
CAGCAGGCGGGAACGTTTAATTCTGCTATAGCTGCGCCCACAGCCACCCCTTCCCCCAGGTGCTCTGTCT
CAGGGAGATGGGAGTTTGATCTATAAGCTACTGACTGGGGCTGCTGCCTTTCTTTCAGAGATGCCCAGCC
CAGAAAGGAGGAATCTAGAGAGGCAGTCTGGCTACAGCGGCTTTGCTGAGCGAACATCCCTGCGGCTTTG
TTTACGCTGTCAGGGGAGAACCGCCTTCTCAAGCCTCAGTAATGGGGGACGCCCCTCCCCCCACCAAGCT
CAAGCATCCCAGGTTGACTTCAGACTGCTGTGCTGGCAGCCAGAATTTCAAGCCAATGGATCTTAGCTTG
CTGGGCTCCTTGGGAGTGGGATCCACTGAGCTAGACCACTTGGCTCCCTGGCTTCAGCCCCCTTTTGAAG
GGAGTGAACGGTTCTGTCTCGCTGGTGTTCCAGGTGCCACTGGTGTATGAAAAAACTCCTGCAGCTAGCT
CGGTGTCTGCCCAAATGGCCGCCCAGTTTTGTGCTTAAAACCCAGGGCCCTGGTGGTGTAGGCACCCAAG
GGAATCTTCTGGTCTGTAGGTTGCGAAGACCATGGGAAAAGCGTAGTGTCTGTGCTGGAATGCACCATTC
CTCACGGCACAGTCCCTCACGGCTTCCCTTGGTTAGGGGAGGGAGTTCCCCAATCCATTGTGCTTCTTGG
GTGAGGTGACACCCCACCTGGCTTCTGCTCACCCTCCGTGGGCTGCTCCCACTGTCTAACCAGTCCCAGT
GAGATGAGCCGGGTACCTCAGTTGGAAATGCAGAAATCACCTGCCTTCTGCATTGATCCTGTTGGGAGCT
GCAGACCAGAGCTGTTCCTATTCGGCCATCTTGCCAGCCTGTGGAGGTTTTAAAAACTATTTTTTAATTG
TAGAAAGTAACATATTGAAAAGCATATAAATACGATTTATACTTTGGTGAGTTATTAAAATGTGGATAGA
TAGTCATTGCCACACAGGTCAGGAAATAGAATATTGCCAGCATCTTGGTGCCATTCTTGCACACCTTCCC
AATAGCACATCTTCCTTTCCTCTAAAGCAGGGTTCCCCAAGCCCCGGGCCACAGACCGGTACCAGTCCGT
GGCCTGTTAGGAACCAGACCATACAGCAGGAGGTAAGTAGTAGGCCAGTGAGCATCACTGGTTGAGCTCC
GCTCCCTGTCAGATCAGTGGCGGCATTAGATTCTCATAGGGGCATGAATCCTCTTGTGAACTGCACATGC
AGGGGATCTAGGTTGCACGCTCCATATGAGAATGTGACTAATGCCTGATGATCTGAAGTGGAACAGTTTC
TCATCCCACAAACACATCCCCCCCATCCCTGCCCCCGCCCTGGTCCATGAAAAAATTGTCTTCTGCAATG
GGAGATGGGTGCACCAGAATCTCACAAGTCACCACTAAAGGACTTACCGTGTAACCAAATACCACCTGTT
CCCCCAAAACCTATGGGAATAAAAAATTTTTTAAAAGAGAAATATCTGAAACAGTAATTACTGAGAATTT
TCCAAGTTAATGTCAGACACCATACCACTAATACAGGAAGCTTTCTTATTTTGAATGGAAGCCGGAAGAA
AAATGCACTTTACCTGTAGAGGAGCAGAACATTCAACTTCTCCTCAGAAACCATGTAAGGAAGAAGAGAG
TGGAGTGAAATATTTAAAAATATTGATTTAAAATGTTGAGAGAAACACCTTACATTATTAAGAACATGAG
CTCAGATACCTCTAGGGATCTAGATTAAGAGAGCCTTTGAATGACTGAGTTCTACCTGTCAAGACTCAGA
TTCCCAGCAGGGTCAGATATGCCCAACAGGTGTGATCATCCCACATGTGGCCGATGGCACAGGGTACTTT
AATTAACCAGATGCTGGCAGCAGCCATCACCCACACTCCCTGGTCATCACAACTACAGCACCATGTTTTC
ACCCCTCCCAGCCCCAGAGGTTGTGGAGCAGCATATTGAGGATCAGTGCACTGCATGATTCCTAAAGCTG
TTTGCATTTAAAACAATCAGATACATTTCTATTGCTGAGCGAAGGTTGTTTGCCTTTTTTTCTTTTTTAG
ACAGTCTGGCTGTCTCACACAGGCTGGAGTGTGGAGTGCAGTGGTGCTATCCTAGCTCACAGAGGCCTTG
AACTCCTGGGTCCAAGCGATCCTCCTGCTTCAGCCTCTAGAGTAGCTGGGACTATAGGCATCTGCTATTA
TGCATGGCTAATTAAAAAAAAAAATTTTTTTTTTTTAGAGATGGGGTCTTGGTATGTTGCCCAGGCTGGT
CTCAAACTCCTGGCCTCAAGCGATGCTCCTGTGACCGCCTGCCAAATGCTGGGATTACAGGCATGAGCCA
CTGTTCCAGGCCTGCATTTTTTTTTTTTTTTTTTTGAGACAGAGTCTCGCTCTGTTGCCCAGGCTGGAGT
GCAGTGGAGTGATCTCGGCTCACTTACAAGCTGTCTCCCAGGTTCATGCCATTCTCCTGCCTCAGCCTCC
TGAGTAGCTGGGACTATAGGCGCCCACCACCACGCCTGGCTAATTTTTTGTATTTTTAGTAGAGATGGGG
TTTCGCTGTGTTAGCCAGGATGGTCTCAATCTCCTGACCTCGTGATCCGCCCGTCTCTGCCTCCCAAAGT
GCTGGGATTACAGGCGTGAGCCACGGCGCCCAGCCCCGGGCCTGCAATTTTATTTGAATAAAATGTTTGT
CTCTTTTATCAAAAAGTTCTTCATTTTACTGGGTTCCCAAATATCTAAACTGATCTGCATCTAGTGCCAT
ATGAAATATATAAGAAATTCATCAGTTTGTTAGTTTCTTCAGTTTCTTTGTTTCTTTTTTTTTTCTTTTT
TTTTTTTTTTTTTTTGAATCAGTCTCACTTTGTCTCCCAGGCTGCAGTGCAATGGTGCGGTCTCGGCTCA
CTGCAACCTCTGCCTCCTGGGCAAGTGATTCTCCTGCCTCTGCCTCCCAAGTAGCTGGGATTACAGGCTC
CTGCCACTACGCCCAGCTAATTTTTGTTATTTTCAGTAGAGATGGGGTTTCACCATGTTGGCCAGGCTGG
TCTCAAACTCCTGACCTCAGGTGATCTATCCACCTCGGCCTCCCAAAGTTCTGGGATTACAGGTGTGAGC
CACTGTGCCCCGCCTCTTCAGTTTGTTTCTTTAAGGACACAGACTTTAGAGCCAGACCCTGGATTCATAT
TCCACAATCATAGCTCTGCTGTTAACTAGCAGTTACTTCCTTTCAGTTTTTACATAATCTTACTTTGGGC
AATTATGTAATCTCTTGGTGCCTCTGTAAAATGGAGATGATAATAGTACCCATTTCATAGTGTTGCTGTA
ATGATTAAATTAGTATGAATAAAGTATTTAATATATTGCCAAGAGCATAGTGTAAATGTATGCAAGTATA
AACTATTATTATATTTATAAAATGAGATATTACTAATTAAATTTCACACAGGCAACATAAAAATACCTTA
AAATTTTTTTTGGTGGGGAATTACCTTTTAAAAATTAAATTACTGGCTGGGCACGGTGGCTCATGCCTGT
AATCCCAGCACTTTGGGAGGCAGAGGAGGGCGGATCACTTGAGGTCAGGAGGTCGAGACCCGCCTAGCCA
ACATGGTGAGACCCCGTCTCTACTAAAAATACAAAAATTAGCCGGGCGTGGTTGTGTGTGCCTGTAATCC
CAGCTATTTGGGAGGCTGAGGCAGGAGAGTCGCTTGAACCCAGGAGGTGGAGGTTGCAGTGAACCAACAT
CGCACCACTTAACTCCAGCCTGGGAGATAGAGTGAGACTTCGTCTAAAAAAAAATACATAACATAAAAAT
AAAATTAAATTACTTCATCTGGAGAAGCGTTTTGCTGTGGCATTTAGCTTAGAACTGTACTATGAGGAAT
ACCGGTGAATTATTCATTCCTTAATTCAGTATTTTTTGAGTGTTTGCCAGGCATATGCTGCATTCTAGGA
AGACAGGGAATTTACACACCAGTAGAGGAAGCGGTAACTAAGTGAACGAATAAAGCCCAGGATAATTAAC
TGGAATCAGCAGAAAGGATTTGTTGTGTGCATGAAGCAGATGAGAGGCATGGGCTGGACCGTGGTGAGCT
GGGGAGAGAGGAAATAAGGGCTGTCAGAGACGGTGCTGGAGCCATGTTTTGCAGGGTCTTCAAGACTCCA
GCAAGCAGCTGGGAGTATCTACGGGGAGTTTAAGTTTCTTTTCAAGGAAGCGTGAAGAGAGGTTTTACCC
AGGGGAATGTGCTGGTCTCATTTACATTTGCTAAAGTTTTTTCTGTCTACTGCATAGAGAATGAATTGGA
GACGGGCAGGCCCAGCCTGGCAGTAGACAAGTGCAACACTCCAGGGCTAGGCTGAGGGGTGGCAATGAGG
TGGAAGGAAGTGCATGCAGTCAAGTCGGATCACACACATTTATTTTGGTGGTAGACTTGACAGGTGGTGA
TGATAGCTTGGATGAGGAAGACAATGAGGAGGCATGGACATTATCTAGATTTTTGGCTAAGTGCCATTTT
GACAGTTGCATATATTCCCTCTATTATTAAACTTTCTTTTCATTTAGACATGATCCTTCTCAAAACTGTA
AATGTTCTAGTGTGTGTGCTGCCCAAGTGAACATGAAGACTGCACATTTTTTAGGTATTTTAAAACTTTC
CCGATCTAGATTTTTTTGCACTGCAGCCAAATTGTACAGTCGTTTCAAATTTCTTCTTTAGTAATGGGAA
CCTCCTTTGTTGATATCTTGGGTATTTAATTGGGAGCATTTTACCTGTTAATTATGGTTGTTGAGGATTT
TAAACTCACTTTATTTACCACTAGTCTTTTGAAATTACATTGCTGAATATCCTGCCTGCCCTGGGGAGTG
TCAGAGCCTGTGAAGGCCTGCACCTGTTGAGAGGCAGCCTTCCCTCCTCTGCGATTCCTGCTTCTGGCTT
CCATGGGTGATGAAGGGTTGTGGGATGGGAGGGGCAGCTGGAGCCGCTGAGCCAGCTCCACCTGAGTCTC
ACTGTGTAACTCCATTGTTCTTTTTAGCATGGCCTTCAGGGGTAGGAAGTCAGGCTTATGAGATTTTATC
TACTGTCACTGGGACTAGTATACCTCCTCAGTAAGTGGAATTTATTATTATTTTACTAAAATATGGTTTC
TTCGTGTGAATGAAGTGAGATCAAGCATATAAAGCAATCTTTAGTTAAAAGATTGCTTAAATTGTTACAT
GTGGAATAAATTATTTTACTTTTTTCTAAGAAATGAAGAGAAACTGGGAAAAAAATTCGTTAGACAGTAA
TGCATAGAGATTTTTTTACACCTAAATGAGAAGCCAGAAATTCATATAAAACATAAGAACTTCAAAAACA
TTTCTTAGGCACAGACTATTATTAAACACTCATGGATGTTTTTTATTTTCTATTACATTAAAATACTCAA
GTCAACTTCATATTCAGAACATTTAAAAATAGCTCAATCTTAAAAAGAAGATATAAAGTATTATCAGCTG
TGCTTTTTGTGCATAATTAGCGTGTGCCTGTGGAGGTATAAAGACTTCTAGCCTAATGATAGTATCATGA
TAAAAAGTTCAGATGGTAAAAATTGGAGCTCCAGCTGGATGACATATTTTATAACAATTCAAAGATGGCT
CCAGCATAGAAGCCATGCTATTTTAGTTCTTTGTAATCCCTCCTTCACCCATCAATGGGTCGTGAAGGCA
CTGATAAAAGAGCCTTTTAGAAATGCTTCTATTTGTTCCCTTTCTTACAGATTTAATGTGTGCCGACCAT
TTCCTTCAGTGCTGGACAGGCTGCTGTGAAGGGACATCACCTTTTCGCTTTTTCCAAGATGGCTCAAGAT
TCAGTAGATCTTTCTTGTGATTATCAGTTTTGGATGCAGAAGCTTTCTGTATGGGATCAGGCTTCCACTT
TGGAAACCCAGCAAGACACCTGTCTTCACGTGGCTCAGTTCCAGGAGTTCCTAAGGAAGATGTATGAAGC
CTTGAAAGAGATGGTAAGTAGTGGACCAGAATAATGAAATTATTTTCTGACTTCAGGGACTCTACCAGAT
TTCACCAAGACAGAATGCCACCCAGAATCGGGACTTGTGGTATTGACTGAAGTGCCGATGGATTAGGAAG
ACAAATTACTCAAGAGCTGGTTTCCTAGTCTCCGATTTATTATATTATTATAATTTCTTCTTGAGTAGCT
GTATTGTTACATGACACTTCCTGACCAAGCAAGTTGTTCTTAAATAATCAAGATTTCATGCTTGTTGTCA
AGTAGTAAATGGGATTCTTTTGAAGGCCTTTGTGTCCCTTGAATCTTGGAGAATTTGCTACATGCTTAAC
CCTTCTTCAACCCTCTTGTCAAGTGGGCTGTCCTAAGAGATGATAGATATTAAGATCTCTTCTTATAATA
CTTTTTATAAATATTAAGATTTTGAGTATCTTTACTATTATATAATTTATTATTTTCAAAATTTAATATA
TTTTCATTTTGATGTCATGTTTCTTTTATTATGATAGATATTTTTATGATTTAGCACATGTTGTTATATT
ATGAGCTAGTATTTTGTATTTAATTTCAAAATGAAAAGCAAAATTGTCACTTAACAAAGTGTTCTTGTGT
TAAGCTTTTCTTCCCTTAATCAAAGAGTAAATAGGAAATTTGAAGAATAATTTGTATTGATAATATGAGT
TCATATGTCTTTAGAAACTATAAGGAAAGATACAGTCTGTGTGAACAACCGATGCTAATTTTTAGAATTG
TTTTTTTAATGAGACATACGTAACTTGACCTTTATATGGTCCCAAAAAGTGTAGGACCAGGCTTGATGCT
GGGACACATGTGAGAGATGTTTTGCTGCAGTAGCAGAAAGTGCCCATTGTGTCGGCTTACCTGCATGTTA
TCCTAGTTTGCTTTTATTTTTGAAATTTTATGAAAGGTAACAAGTGGAAAAAGGTCCGGTTGTACAATAG
CATGCATGTGTGTTTTTAAATCTTAAGCTGTTTTCTATTTGGTTGATCCTTTTTCTATTTCAGGGTTTAG
ATTGTTACAGACTATGATGTCATTTGCCATACAGTACTCAAAATAGTAATAAACAGATATATACATTTAT
GCTTTGGCTGCTTTTATTTCTTTTTACAAGTTCCAGAGTTCTCATCCCTTTAAACCTTCTTCTTTGAAAT
AGTCTTACTGGAGCTGAGTTCGTAACCTCTCTTATGTTAGAATAAAAAAACCAGGATAAAGATACGGGAT
TATATAGTTATTAATCACCAATAATGTGTTTTCAACTCTTAAGCAGTCTTTATTATTTTTAAATAAAGTA
GCCTTTACTGACTATTAAGTATGAGTAGAATTAATTTTGCAACACAATTGATTTTACCTATGTCATACAT
CTTAGTTTGGTGACCCCATTTAAGGATGAAGTAAGTTGGTTGGTTGCTTTTTAAACTTTATTTGAATTAC
TTACCTAAAGATCTTCTCTAATCCTTTTGTGCTTACTTTTAAAAATGGTATACTAAACAAGAAGCATTCA
CGTTCCCTCAATCTATAATGTCAGTTCAGTATTTCTAAGTTGCATAATGCCTTTACTGACCAAAATTTAT
TTTTCTTTCACAGGATTCTAATACAGTCATTGAAAGATTCCCCACAATTGGTCAACTGTTGGCAAAAGCT
TGTTGGAATCCTTTTATTTTAGCATATGGTAAGAATCAAAAACGTGTCCTCTCAAAATGGCTATTTTAAT
CTTTGCATTGTTTCACAGAGGCTTACATTATGAACCTTTCTCCTAGTTTTTTTTTTTTAATGGAACAACT
TATCAAATGTATTCATTGTTCTATTACAAAGATGGCTTCCTATACTGTTGTCCATGTATGTGTGTGTAGG
TTTGTATAGATAAACACACACACACACACACACACACGCGTGCACGCACCCATGCAGATCTTCAGGAAAA
TAGAGCTCTTCGTATTAGAGGACAGAAACTCCCAGATGATTTAAATATCTACTTTTGCCTGTAATCACTT
TCATAACTTCATTTTAAATTCAATATAAAATGGCTATACTAATGATACCGAGTTAATTTTGTTTCACGGT
TTCATAACTCACACACTCTTTCCCATGTTGTAAAGTCATTCTTCAGAGTGTGAATCCTGCCGTGACTTTG
CCTTTGACATTGGCTGCTTTGACCTTATTTAAGGGTGCCATTTTGGTTTTATCCAAGGTGGAGATGAATG
TGGGGGCACTTTGGGGAGGTATTCCTTCACTGAGCTCCTGTCTGCTGTCCAGTCTCATTTCCTGTTTCAT
CTTTCACAGCTATACCTCATGGTGGTAGCCAGGTCCTATTACTAACAGTGCCTGAAGGCACTTTGTGTCA
GGATTCCTTTCACACTCATTCTCCTTTGAAATAGCCTTACTGGCCTGAGGTAGGAGCTGCTGGGAGTGTC
TCCATGGCCTCCAGCATCTCCCTTTAATACACTTACTTCACTGTCTGATGCCCTGGGAAGACAGCATGCC
TCTCGAGAACAGGAACCATGGTTTTAGTTCTCTTTGTTGTCCTAGGGCCTCCCAAGGAACATGGCTGAGG
TTTGCTTTGGATTGACTGTATTATTAGCTAATGTAATATTTACTTTTAAGATTTTTTAAAGTCAAAGACT
AATTGCATAATATAATGACATTAGAACCCAAAATAAAATGAATATTCCCCTTTGATGTTTTAAAGAATCA
TGCAGTTTCCCATTTATAGGTTGTCATAATCATTATTGGAAGAGACGTGAACTAGGCAGGAAAGGGACAA
TATGTGTGTTTAGCCCTGTGAGCAGGCGTGCGTGGACGCCCACACACTCAGCCTCCCTCACTCTACCCCA
CCCTCGTATGTCCCTCCTCCTTCCCACTTATACCTCTTCCCCCAACCCCTAAACTTCTTCAAGTTCTTGA
TTCAAAATGGCAAAACCAGTGAGGCAGCAATGTGTTTGCCCACCAGGACCACGCTTACCTGTCCTAGGTT
TATCTATTGCAGTTAGGTCAGTGTTTCTCAGACATCTTTGACCAGAAATACATTATGTATATGCATATGC
ACGTGGATATGCACACACACAGTGAAACACATCTCATGAAAAAATGCATACAATGGCACCCCCCTTAGAC
TTTCTGCACTTGCAGTTACTCACAGTATAGTACAATAAGATTTTGAGAGAGATGGAGAGATCACATTTAT
GTAACTTTTTTTTTTTTTTTTGAGACAGAGTTTTGCTCTTGTCAACCCAGGCTGGAGTGCAATGGCACTA
TCTCGGCTCACCGCAACCTCTGCCTCCCGGGTTCAAGTGATTCTCCTGCCTCAGCCTCCCAGGTAGCTGG
GATTACAGGTGCCTGCCACCACACCCAGCTGATTTTTGCATTTTTAATAGAGATGGGGTTTCACCATGTT
GGCCAGGCTGGTCTCAAACTCCTGACCTCAGGCGATTCACCCGCCTCGACCTCCCAAAGTGTTGGGATTA
CAGGTGTGAGCCACCGTGCCTGGCCTAAGGGTTTAACAATGACAACAATACAATAAATCAACTGTAACAA
TATAGTGTAGTACATAACTTCTATTACACTATATTGTTACAGTTGATTTATTGTATTGTTGTCATTGTTA
ATCCCTTGCTATGCCTAGTTTATAAGTTAAACTTTATCATAGGTATGTGTATAGGAGAAAACACTGTATT
TAAGGTTTGGTACTATTCTCAGTTTCAGACATCCCCTGAGGGTCTTGGAATATTTCCGCCATTGATAAAG
AGGGACTATTGTATTATTATTCTGTGCAGTCCATGTTCACTATGCTGTTCATAGTGAATGGTGTTCATGA
CCCGAGACTGGATTTCATAACCCTTGATGCTTCTTGACCAGAAGTTTGAAGAACATTGATCTAGGCCAAT
ATCGATGGCAGGAAAAAGGAAAAGAAATTGTTGAATGACCTTCATTTGAAAGCAGTACCCATTGTTAGGG
GCTTCATGGATTTATTAATTGCTTTTTAGAAAGAAAGAAATATTACGTCCTTTAAATGTACATCGTGGAT
GTAATACATCCTCAAGTCAGAAATGTGAGAAACGTGGGGAGGTTTATGATGTCGTGTGAGAGGCATTGCT
CACTTTGAGCTGGGCACTGTTCTCTGTGGGCTACATAATGGACCTAATTAATTCTTGATGACAGTGTTGC
AGGGTAGATAATTATGGATTAGAGTCAATAAGTCGCTTTCCCTAGAATACAACACTAAGAAGAGCCAGAA
CCCCACTCCTCATGTGACTTTAAAGTCCACCTTCTGTAATGTTCTGTCCCTATTGGATCACATCTTGTGA
TCGAAGTGTTCCAGTCTTGGCATTCCCACTAGCCAGCTTCACTTGCACATACATATTAATTCAGCAGGTA
TTTACCAAGTGTTGGGTTTTTTTTTTTTTTTTTTTTTTTTTTTTTTTTTTTGAGACAAAGTCTTGCTCTG
TTGCCTAGGCTGGAGTGCAGTGGCGTGATCTCGGCTCACTGCAACCTTTGCCTCCCAGGTTCAAGGGATT
CTCCTGCCTCAGCCTCCTGAGTAGCTGGGACTATAGGCACCCACCACCACATCCAGCTAATTTTTTGTAT
TCTTACTAGAGATGGGGTTTCACCGTGTTAGCCAGGATGGTCTCAATCTCCTGACCTTGTGATCTGCCTG
CCTCGGCCTCCCAAAGTGCTGGGATTACAGGCGTGGGCCACTGCACCCAGCCTACAAAGTGTTTTTTTGT
GTGTGCCAGCCCTTAGGAAAACGCGGCTCACTGTCTCTGCCCATTAGGAGCTTCCAGTCAGGGAAAAAGA
GATACTTAAGCAGGTCAGCCTGACTCAATGTGATGGTTCTCTGAATGGGAGTAGCAGTTGCTGAGAGGAA
GGAGGGTTTCTCAGGAGAGCCCCTGCTGGGGCAAACTTGAAGATGTGATTCTCTAAGACAGGGCATCTGA
ACTCGTGGGGTCTCATATTTCTTCTTTGTAAAAGTAGGAGTTCAGACTGATTTATTCTGGTGTCCTTGAA
CTTGTATCTGTCTGTGATCTTTCCTTAGCACCTTATCTGCATTACCTTGGCCCCTCTGACAACCAGAAAC
AAATTCTCCATCCCTTTCTGTCTCAACGCTTGTATTACCTTTTGAACTGCTCATAAATTTCAGTTTCCTG
TTGAGGTATGCCCTGGCTGTGAGCTCACAGAGGTGGCTGAATGGCATGAAACTCCATTAGCAGTCAGATG
TTGCCTGTGTCGCCCCTGTTGTTTGACTAGTTGGGCGCTTTCTTCCTGGAAGGGATCTTTTCTTATGGTT
TTATAATATCAGAATTGCTTTTTTTTGAGATGGAGTCTCGCTCTTTCACCCAGGCTGGAGTGCAGTGGTG
CGATCTCGGCTCACTGCAAGCTCTGCTTCCTAGGTTCACGCCATTCTCCTGCCTCAGCCTACCAAGTAGC
TGGGACTACAGGCGCCCGCTACCACGCCCAGCTAATCTGTTTTTTTTTTTTGTATTTTTAGTAGAGATGG
GGTTTCACCGTGTTAGCTAGGATGGTCTTGATCTCCTGACCTTGTGATCCGCCCACCTCGGCCTCCCAAA
GTGCTGGGATTACAGGTGTGAACCACCACGCCTGGCCCAAAATTGCTTTTTAATTACAGTAAAAGCAAAC
AATTTTCATCATGGAAAAAAGTAAGTCATATGTAGTGTTCATGGAGAATAGGATCAGAAAAGGGTAATAA
GGAAGGAGGGATGATTTGGAAATGAAAAGATACTGAAAAGAGAAGAGACAAAATAGATAATAATGGAAAG
TTAGAGAATACCTTTTTATTCGATGATGTCCTCAGCATAAAAATACTGCCTTCACCTTCTGAAATTCTGA
TCCATTGCTTCTAACTCTGCCCTCACGATTAAGCATGACAGCTGTTACCTCTCTTCTTCACTATTCACTA
TTCCTGTATAACAAATTTCTGCCAAACTTAGCAGCTTAAAACAGTGAATATTGTCTTAAAAATTTGCTGC
CTTCATACTTTTAAACTATAGAATTAAAGTTTAAAGAATTGCTTTAGCTTTTATGTAGAATTTCAATTCC
AGATATCAAAAAATAAAATACAAGATGGCAAGTGTATGTTGTTATCTTCCTTTCCTTTCCACTCAGTTTT
AGAATCCAAATTTACACTAAGATGGGCTGTCAGTGACTGTCCCTGCAGTGAATCCCAGGCAGTTTCCCAT
CTCACTTAACATGCCCTGTGGGATGCTCCAGCCTGAGCTCAGGGCCTCCAGGGAGGAGCCTGACAGATGC
CAGCCCCTGGAGTGAGTGCTGGTGGCACTTGTGAATAACAGTCCATGGCTCATGTTTGTAAATTGCCTAA
GCAAAGCAAACTGTAAGCTCATTAGCAGTGCATTTCAGTGCATTGACTCTGGGCACAGAAAGTCACCTCC
TACAGGTTGGGTGGAAGTTTCTCTGGCTCCTTCTCTTTTTTTATTTTTCTCCCAAAAAATAAAATTTTGG
GAGAAAAATAAAAAATAATATACCTTTTGAACTAAGTTTGCATAATCACTCAACACCTGGGGAAACTCCC
AGTTTTGGGTATTCTTTTAGATTTAAGCTTCATAAACTCATAAACTGACATTTCCTTTCTTTTTTTTTTT
TTTTTTTTTTGGTGGTGAATGTCTGGTTTTTGGAAATGCTCTCCATCAGATTACATTCTTTTTAACACCC
TGCTGCATGCGTAAGGATGCCATTGGATGCATCTGTCACACTTACTACATGAAGCTAGGTAGAATTTCTT
CTAATATTGAAACTATGGGAAGCATAACAGTCACATTTTTAATTAGTGGCATTGGTTAAACCTGAAGAAT
CCTAGAATCTTTAGTGGTTAAATTATACGACTCTTGAAATGAACTGATCTCAGGCAAGCCAGTGCTTTAT
AATGCTTTATTTGAGCTCATAAAAGCTAGGGGAAAAAAATGAATGGCCTCAGAATAACCCCTGATAAAGT
TTTTTCAGAGATGTTAGTTATTGGAAAAGCATAAGCTATGTATGTTCACAGGGAAAAAAATGGGTTTAAA
AATTACTGTTAGCATTATTTAGTTTGTGGATAAAAATACAGAAGTCGCTTACTAGAGTTGGTATTGTTCT
TATTTCTTTGTTAATGAAGTCCTTAGAAATGCTGGGTTTTGGCCAGGCACGGTGGCTCATGCCTGTGATC
CCAGCACTTTGGGAGGCCATGGCAGGAGGATCACTTGAGCTCAGGAGTCGGAGACCAGCCTGGGCGACAT
GGTGAGATCCAGTCTCTACAAAAAATACAAAAATTATCCGGGTGTGGTGGCACACACCTGTGGTCCCAGC
TACTCAGGAGGCTGAGGTGGGAGAATCGCTGGAGCCCGAGAGGTGGATGTTTCACTGAGCTGAGATCATG
CCACTGCACTCCAGCCTGGGTGACAGAGCGAGACCCTGTCTCAAAAAAAAGAGAAAGATTGGGTTTATTA
GATTGCAGAAGATGAGGTGGCAGAGAGTACTGATTCCAGGCACCGGCTGTCTTAGCCTCACCTCCATGGC
CTTGTAAAGTCTGAAGTTGCTTTGCCCTAGCTCGGGTACTGACATCTTCAGTTTCTAGGCCCCATTTTTG
TTTCTTAATTGTGTTATAAAATCATCCGCTGGAATAGACTCAGTCACCACAAAAGGAGAAAGAAAGTAGC
TCATCAAGTAGATGTTCCTGCAAAATGTAAGATTTCAAAGTTCAGATTTTCATTGCATGTTTGGTAAAGT
TTTATAAAGATTTACATTTTAGAGTAAAAGAGCATGTAAGAGACTTCTATTTTATCCAAAGCACTTGGTA
CTAGAAATATATTTTAAAACAATCCTTTTTATTAAATTTCTTTTTAACAAGTGGAGCTATTTTGGCAAAT
TAAAAAATTAAAAGATTAAATCACAAATACTGGCTACCAACTGCCTATAACCATTAAGTTGCCTTAGAAA
GGTCGAAATTACTAACCATTTGAAATGTGAAAAAACTGATCATGAAAAAAGCCCAGTTCTCTTAGTTCTT
TCCATCATTCTCGTTTGGCTTTAAAAATTGATACTGTTACAGAAAGGAAATTGACCACACAAGAGTTAAG
TGATCCCAAGGCCACAAGAAGGACAAAACCCCACAAAGTTAAGTTTAGGGAATTCTGAAGGACAGCTAGG
AGAGCAAAGATAGAGTGAGGGAAAATATTCCCATCAGCAGAAAGAGAATGTGCAAAGTATGATGGCGAGA
AAGCAAATGTTATATTCAGGGATACTTGTTCTGTGAACTTAGTAGTTTGAGATTTTCCTAAATATAATGT
TTACAGTGTTTTTATATTAATGATTTTTTCTGCTTGATAAAACTTATTAAGTTTTCCTTTTTGTAGATGA
AAGCCAAAAAATTCTAATATGGTGCTTATGTTGTCTAATTAACAAAGAACCACAGAATTCTGGACAATCA
AAACTTAACTCCTGGATACAGGTAAGAGAGTAAATCTTGCTCTGCACTTCTTTGAATTAAATTGATTATT
TAAAAGTGCTGCTTAAAAAAATTTTGGGGTGGGGTGGAATCCAGTTCTATGATGTTTTAGAAACTTTTTC
AAACATACCCTTCACTGTTACATTATCTTGCTGAGTAAAAAGTTTTTCAAGAGATTTCAATTTTGCTTGA
CTGTGAATTCCAAAAGATACCAGTCTTTAGGATGAAGACCTAATGTTGAGTGGTTTTGAAAAGTTTCTTT
CAGGTGATTAAAATAATGTATTCCATTTCACTCTAGCAATTGCTATCCTTCAAAAAATTGTTTTTCCCTA
TCAACCTGGTTTAAATTCCTTTCCACATGCTTCCATGCCTCTCTCAAAATCTATTTTGTAGTGAAATGAA
AATTCAGCAGTGCGGCGGGAGCAGCACTCTTTATTTTGTGTCCCGAAGTTGGTGCCTGAGCCAGCATATG
CACAGACTGGATCTTGGTGCTGCGCAGCCTCTGCTGCTCACTCACACTTTCCAGTTGGCTACCTACTGTG
CTAGTCCAGATAGCTGACAGCTTTCTAGATTTCCCTGTCTGCTTCTACTTTCTCCTGAAGGGTAGGCATA
AGGTAGAGATGTGGTGAGGCTGGGCTGCAGCTGCCCTCACCAGCAGCGCATTGGCTGTTCTTTTCTGTTC
CCCGCACTGCCTTGCTGCAGCAACCCCAGGACTGGCTGATGCCTGTCAGACTCCACTTAGTATGCTTGTG
GTTCAGATCAGCTGCTTAGTATTATGAAATCATCCAACCAGATTAGTGCATATGGTTTTGTCTATCAGTC
TGACTGGTGATATCGTTACATTCACTGCTTTTTACGATTTCTTTGTTTTAGACAAAATTGCTAACAGATG
ATGAAGACAGTATTTATATTTTTCTGTTTGAAAGATAGAAGGGAGTGAGAGGGATTGGGCAATGGAAGGA
AAGAATACAAACTTTAATTGGAAGGAAAATTAATGTAAGGAACTGAAGAAAGGAACAGATAAGTAGAATT
AGGGAAGAAAGAATGTGTAACAAGGCTGAAGAAACAAAATGACATATTTAGTCCTTAATTTTTTTAATTG
TTGCTTTTATTATTTGATATTTTAATGAATATTTAGCATATAATTTTGTATTAGATAGCTCATTGAGTCC
TGAAGTAGGCCATCTAATGAAGTGTTTTTAGAGTGACTGGATTAACTTTCTTCATTTAACTCATGACTTT
ATTAGCCTGTTTAAAAATTTCTAGCCTCACATTTTATTGTTTTTCTTGTTTAATACTATATCTACTTACT
AGTAGGTACTTATTAAGTATAGTTCAGGGAGTTAAAACACTTAATCTGTGCCTTCGGGGAATTCCCAGAC
TGGTAACCAGGTCACTCCAGGGCAGCGTGGTTGAGTTATTGCTAAAGCTGTGTTTAGGTGTGACGCTGAC
GTAGAGGGGGACACTTGCTCTGACAAGGGGGTTTGGAGACATTTCCTGGGGCAGGTGACGTGTTAATTGA
ATTCTGAAGAACAGCTAGGAGAGCAAGGATAGAGTTAGGGAGAATATTCCTGTCAGCAGAAACAGACTAT
GCAAAGTATGGTGGTGAGAAAGCAAACGTATTCAGGGAACTGTGGGTACTTAAGTGTGCCTGGAACTTGG
GGTTTTGTGGAGTTGGTGATGAAGCATGAAGCCAGAGAGACCAGCAAGCACAGTCCTTGCATATGACTAT
AGAAGGTTTTTATCTTGATGACAGTGGGGCAGCCACCAGAGTATTTAAAACAGAGAAATCATGCCTGGGC
GCGGTGGCTCATGCCCCAGCACTTTGGGAGGCCAGGGTGGGCAGATCATGACGTCAGGAGATCAAAACCA
TCCTGGCTAACATGGTGAAACCTTGTCTCTACTAAAAATACAAAAAATTAGCCAGGAATGGTGGCACGTG
CCTGTACTCTCAGCTACTAGGGAGGCTGAGGCAGGAGAATCGCTTGAACCTGGGAGGCAGAGGTTGCAGT
GAGCTGATATCATGCCACTGCACTCCAGCCTGGGTGACAGAGCGAGACTCTGTCTCAAAAAACAAAAAAA
AAGAAAAAAGGAAAAAAAAAGCCAGAGATATCATGTAGCCAGTGTGAGTTTTAGAGACTAACAGCAAAAG
GAAGTTGGTTGGGAAGAGGGCAAGGATAGACGTTGGAAGCTATTGTAGTGATAAGTTGAGAAATTGCGAG
GGCCTCAAAGAAATGGAGAAGGGGGCCCGATTTATAAGATATTAATAAGGCAAATACCCAGAAGGTATAA
TGGCAGATTAAATGTCGATACACTTAGGCATAGCTGGAAATGTACAGAGAGCAGGTGAATATCCCATTGT
CAGGAGAGCAAGCTGATTTGCTGGCAGAGGATTGAAAGTCAGCCACCTGTGGGAATAGTGGAAGCCTTAG
GTGAGGTAAGGTCCGCTCCCAGAGAGTGCTGGGGTAGGGGCCCAGGGGAACTAGAGAGAGCCTTGGGGAG
ACACTGACCTTAAGGGGAAGTGGAAGAATGATTGATGCTACAGAATGTAAGGAGGGGTCCAAGAAGTAGA
AGGAGAGAAGCAACGGAGAATGAGTGCTCATTCATATCTAATGCCATACAGCAGTGCTGTCCAGTAGGAA
TAAAATACATGCTGCATAGATAATGGTAAACTTTCCAGGTGCTGCTTTAAAAAACATAAAAAGAAATAGT
TAAAATTAATTTTAAGAAGATATTTCACCTAATTCAGTATATTCCAAATACCATCATTTCAACATTTAAT
CATTATTAAAGTTATTGAGATTATTTATAATCTTTTTCATGATGTATCTTTGAAATCCACTGTGTATGTT
ACGCTAACACTTATATCTACGTGTTGTATCTTCATTCAGATGTTAAATTTTCTTTGGAAATTCTTGATCT
GTATTTAGATTTTATAAAATGTAGGGTTGAAAAAGTAGATTCACATACCCAAGTTGTTCCGAGGAATTTT
ATAACTGAATCAAGTATCGGTATTTAAATTAAAATTGAATTAAAATTAAATACAAAGTTCAGTTATTCCC
TGGTGCTTACTGCACGGCAGTGCTCTGCAGCCACATGTGGCAGGCGGCCGCCGTGTTGGACAGGGCAGCT
GTGGAGCATACAGCAGAAGGATCAAGAAACACTACTTGGATTTAGCCACTGTGAGCAAAGACTCGGTGAG
GAGAGGTTTAGTGAGAGGTGGAGGGAAACAGCCAGATTGCTCCAGATTGAGAAATGTTGAAGAGGTGAGG
AAGCAGAGATAATAAACTTCACTCTTTCAAGAAACTTGGCAGTAAAGGAGGGAGAACTTTGGCAATGAAG
GGGGTGATAGCCAGACTAGGGTGGGGTTGGCTGAATTGAGCAAGTCCCAAACATGCTTTACATTGAGATG
AATATGTGGGTAGGGAACAGGTGAGGATATGCGAGGTTGGGAAAACAGTTTCCTTCCTCAGTTACCAAGC
AAGCCTGAACTAAATAACATGAACCGATAAAAAGATGTCTAGGAACATATTAATGATTTAACTCTTAACA
ATTTATTAACATATTAATTATTTAACTCTTAACAACTCTAAAGCTAATTACATTGCCCATCATCAGTCAG
TACATACATAATCTCATGTTGGCTGTAAATGCAGCAAGATAGAAGATAGTCTGTAGGTTGGTTTATAAAT
CAGTTCACATCTCCGGTATACTTGATTTTATCTTGGAGGTGATATTTTCTTCATGGAGGTTTTAGAGTGA
CATAGTATCTGTTAAAGATGTGTACCTTGACATCCAATATCCTTGTGAAAATTATAAAACAGCCATATTT
TAATGTTTGCTAATTCATTCAGCAAGCATTTATTAGGGCCTACCATGTGCTAGGATGTTCTAATTGTGGT
AATAAAATCTTGGGTCATTCTGTGTTACTGGAGCATTACAAGTATGATTGAAATACTCTAAATCAAAAAC
TAAGAATTTCTACAGTAAATTACAAAATTCAGTAAACATTCAAATTAATTAATAAATTTGAAAATGATAG
ACTGAGTTGTATTTTTAGCACAGAGAAGAAATAATTCTGTTTGACACTTGACATGTTAGCATGTGTTATA
CTGGAAACACATTGATAAAATGTTTTGGAGTTTTACTGACAACCTTGAATGTGCCATCATAGAGCACCAC
CTCAACAGCTCTTCTTACAGTTCAGAGACGAAGTTCCGGGACTCTTTCATGTATTTTGGGAATATGTGGT
CTTGAAAAATGTAGTTTTACTGGTAAATCCACTGAACAGATACTTTTTGAGCACCTACTGTTTGCCAGGC
ACAACTATAGAGGCTGAGGATACAGCACTGGACCTAGAAAGACAGTCTGTTCTCAGATGCTACAATAGAT
GGGGAAGGTTTCCAGGGCCTGCCAAATGTAAGAGGATGCCTGTCTTCTCTGGCAAAACCATACCACTATT
ATTTTTTGATTCTGATAGAAAGAAAAAAAGACAAAAAAACACAACAGGGTACCAGGAACAGAGATCAAAA
GGTGTTGTATTCTATCTGTATTTTCTTTCGAAATGCAATACTAAGCAATATCCAGATAGCGTCTGTCCTC
TACTTTCTTCTTTTTTCAAAAAGAAAAAACTTATAAATTAAAAAATCTAACAGACTTACACAAAACATAA
ATGCCAAGCTTAATAAATTGTGACAGATCCTTGTAATCACCACCCAAGGAAATAGAACAGAACATTGACT
GCTCCTGGATGTCCCTGCCCCCCATGTGCCCCTTGTTGCTTTTTTTTTTTTTTTTTTTTTTTTTTGAGGT
GGAGTTTCACTCTTGTTGCCCAGGTTGGAGTGCAATGGCGCAATCTTGGCCCACTGCAACCTCCGCCTCC
TGGGTTCAAGCAGCTCTCCTGTCTCAGCCTCCCAAGTAGCTCAGATTACAGGCATGCGCCACCACACCCA
GCCAATTTTGTATTTTTAGTAGAGATGGGGTTTCTCCATGTTGGTCGGGCTGGTCTCAAACTCCCGACCT
CACGTGATCTGCCCACCTCAGCCTCCCAAAGTGCTGGGATTACAGGCGTGAGCCACTGTGCCTGGCCACT
TTGTGCCTTATTTAAGAAATTTTTCTGCAGTCCAAGGTCATGAAGATCTTCCATATTATCTTCTAAAAGC
CTGTTGGTTTCCTTTTCCTATGTAAGTCTGTGATCCACCTGTAACTTATGGTGTGGTGTAAGGTGGTAAG
TAGTCCATTTTCATCTTATTTCCACGTTGATGTCCCATTGCCTGACACCGTTATTGAAAATCATGATCTG
TGGTGCTACCTTCGTCATGCATCAGGTTCCTTTGGGTGCATTTCCCACTGATCTGTGGTGCCACCTTCAT
CATGCATCAGGTGCATTTCCCACTGATCTGTGGTGCCACCTTCATCATGCATCAGGTGCCTTTGGGTGCA
TTTCCCACTGATCTGTGGTGCCACCTTCATCATGCATCAGGTGCCTTTGGGTGCACAGGTGTGATCCTGC
ATCTCTCCATTGGTTTTGTTGTTTATTTATGGTATTAATCTTAGTATCAACTTGACTGGACACAAGGTCC
TGGTAACATTATTCTGTGTGTTTCTGTGAGGGTGTCTTGGGGTGAGATTAACATTTAAATTAGTTGACTG
AGTAAAGCAGATTGCCCATCCTAATACGGGTGGGCCCTATCCAGTCAGTGAGAGACCTGACTAGAAAAGA
AGGCTGATGCTCCCGAGTAAGAGAGAAGTCCTCCTGCCTGAAAGCTTTTAAACTGAGACATTGTCCTTTT
TCCTGCTTTCACACTCAGACTGAAACGTCACCTCTTCCCGGGTCTAAAGCCTGCTGGCCTTCTGACTGAA
ACTATACCGGTGACTCTCCTGGTTATCTGGGCATTTGCTCTCTTGTGTCTCTGACTTGCTGGCTACTGTA
ATGGTGTTAGCTGATTCCTTATAATAAATCCCTGTGTGTGTGTGTGTCTATCTTATTGGTTCAGTTTCTC
TGGATAACTGACAAATATACTATATTTATGCCAGTTCCACACTGTCATCATTAATGTGGCCTTGTAAGTA
TCTTGCTATCCAGGAGGAAGTTCTTCCATCTTGTTCTTCATCAAGACAGCCTTGGCTAATATTGGAACTT
AGCATTAACTGTTACATAAATTTTAGGATGTGTGTGTCAGGTTTCACTAAAACACAAAATAACTTGTTGG
CATTTTGATTGGCATTGTGATTAGTCAGTTTGGTGAGAATCTCAATGGCCACTATCCTCTAAGGCAAAAT
TGGTTTTTCTCAGTTTTGTTTACTATTAGCAAATTGAACAGTACCTGGCACGGCATAAATTTTTGAATAA
ATTAATGAATATATGATATGCATAAATGAATTAAAAGTTGTACAAATTTATTTTTTCAGATAATCTTCCT
GATAACTTTGAAACATTTTTGCCAACTTTTAATTAGCATAACCTTTTAATGAGGCTGATGATTCGCTTGC
AGAAGTGTCTGTGCTACCATTTCCCAGTTCTCTTAGGTTGCATTCATAGTAGTATTTTTAATCTCTGACT
TCTTTGCAGGGATCTTTTGAAGCCAGGTGTCCATCTGATGGTTCCTTTAGTTAGATAGCATAATCAGCAG
ACCTTCCTTACTGGTCACATGGAGACCATAATGCATATCCTGAGAGGAAGTGGGAACCGGAGGGCTCCTG
CCAGCCCTCTGTGAGGTGGAATCCCCACCCTTCCCTCAGGGTCCCACTTGGACTACTTGTGACACATGAC
CATCTATAACATCTAGACTGGCTAAGGTCGTAGTGTGAGTACATTTGTGAAATATTAATAAAGCTTGATT
TCTTCTTACATATAAATATTCTAATGTTGTTTTCACATCTCAGTGGACTATCTTGTGCACTACTTGGAGT
ACACACCCCAACTTTATTAACCAGTAATAAATTTTGATAAAATCCACATTCTACCTTGGTATATGCAGCT
TCTCTTCATCTTTTCTCAACCCTAATATCTCCATTTTCCATTTTTGCATCCACATATCATGAAGGCCACT
CCCTGATCTTCCCAGATCCTAGCATCTTGCTGCACTAGCTCCAGACATGCCAAACCTATGCACAGATTTG
TACTTGGCAGAAATTTTCTTTCCTCTCCTTGGAATGGTTTTATACCCTTACTAGCCTGGCCTGGCTGAGA
TATGGAAACCTAGGGAAATAATTATTGTTCAAGTTTGAATGGCAATTTAAAAGGGAAAATAAGATGATTA
GTGAAAAATAGCAGAGGCAAGGTGAAATTCAGAGATGAGGCCAGGGATGTTAGCGATTCCTCTTCCACAG
CTCCATCTACTATTTTTATTACTTTTAAGTAACAAACACATTACTGATATTTTTTTTCTACCTGCGTTCT
ATTTGCTTGACTTTCTACAACCTCAGGGTTTTTTTTTTTAAAGATTAATTGATAATTGAATTTTTGTATT
TGCCTGTATAGGGGATATTTATTCATTGCTTTTCCAGTATCCATCTCCCCTCCTTTCCCGCCTTCTTAAT
AGTACCCTGAATTTCCCAGCTGTGCATGATTCATTCTGGGTGAGAGGTATCTTATGTGGCTCCTGGAGTG
GGTCCTACCTGGCTTAACCAACACAGCTTCTCTGTTCCTCTGGCCACACTGGTTGGTCAAGATGAGCCCC
GTCAAGGCTGATGAGATTCAGGGCTGTAACATTTGTTTGCAAGATTGGGAAGTGGATTGTATCTTTCCTG
ATGAACATGAATTAAGTAGCACATAATCCCTGAAGTTGCTGTTGGTCATGTGGAACCATGAGATCAGAGC
GTCTCTGGAAATGGAGTTAGTCCTGACAAAACATAGCTGAGAAAAACAGCAAGAGAAAACAGGTCATATT
CACATTGTGTGGACCCTGGATCAAGTCTTTTCCAGGCATAAATGCTCGAATTTCTTTCCTTTTCTATTTG
AGCTGTTTGGTTCAGGATTTTGTTACTAAAAGCCAGGAGTGTTTTGAGATAACATTGTTTGGGGCATAAA
AAAACATTTCTTTGAAGGTTTTTGAAACTCAGGGACACATGGACACAATTGCAAGAAAAAAATCTTATTT
ATTCAAGGATGTTATGAATAAATAGTGAATTTAATAAAGTACTTTCAGTTTCTTAAAAATTGCTCTAATT
TGTATTTGTTTTTTCACTGCCATTCATTGTAGAAGATATATGCATTCAATAAGGCATCCTTCTTCCCAGC
CTCAGTTAATTTTACTCCTTTGCACAATCCTGAAGTGAGTTCATAATATATGTTTGTCACTGATATGGTT
TGGATCTGTGTCCCACCCAAAACTCATGTTGAATTGTAATCTCCAATGTTGAGGGTGAGTGGGGCTTGGT
GGGAGGTGATTGTATCATGAGGATGGTTTCTCACAAATGGTTTAGCACCATCCCCCTGGTACTACTAGTT
CTTGCGAGGTCTAGTTGTTGAAAAGTGTGTGGCACCTCCCTTGTTTCTCTTGCTGCTGCTCTGGCTGCGT
GACATGCCTGCTCCCCCTTCACCTTCCACCATGATTGTAAGTTTCCTGAGGCCTCCCCGGAAGCCAAGCA
GATGCTGCAGTGCTTCCTGTACAGCTTGCAGAACCACAAGCCAGTTGAAGCTCTTTTCTTTATAATTACT
TCATAAATTATTCATTGCCTCCATTACTCAGTCTCAGGTATTTCTTTATAGCAATGCGAGAATGGACTAA
CACAGTCAGTCAGCTTATGCTCATTTCCATGGTTGCTGAAATGGCATAGACCCAATAATTGGAGGTCACC
ACAAGATCTTGGCAAAAGAGAGAGTGGCCTTCAAGTAATACCTTTATTCCATGTTATAACTTACCAAATA
ATAAATAAGTTTCTTTTTAAAATATCCCTCTCTTTGATTTGCACCCTCATAAGAATTCCTCTTTAGGTTA
ATCACGAACTCTGTTCTCCACACTTGGAACTTTCAAATATAGGATTGTTGCAGGACTTTTCCCTAGTTCA
GCTAAGGACGGGGTTCTTTATCCCATGGCCATGAAAATTCAGGCTTGTAGACAATTTGAATGGTGAGTGA
GACTGGGTTTTATTGGGTGAAAAGGAAGAAAATGGGGAGACAGGGGCTCTCGCTAGACCAGAGTCCCTGC
TGGAGTGCTTCCCGCCTGCCATTCGAATCCCAGGGTCCTCACAGGAAGAGGAGGGGCCAGGCGCCTCCCT
GCTGCAAATGTTGTGAACTTCCTGAGGCTCCACCTCAGTCAGTGGGCAGGCTGGTTGGAGTTTCTCCAGG
GACCCCTGTCACCTGGCTGTCTCAGTATGACCTTGATGTATCATTTTTAGGAAACGTGTTTGCAACCACC
ATTGAAAAAGTAACATAGTTTGGATATTTTATGGACTTGGTTTTTACTTTCTGCAAATATTGCATATAGA
TCTCAGAAAAGTAAAACAGAAGGAGATAAACTCATGGTCTAAAATAGTATTTGGTATGACAGTGGAATTC
AAAGGTTCATACTTGAAATTGAACCTAATGCAAGATCAGACTGGTAAAATGGAGTGAAGTTACAAAAAGA
AATCTGATAATGAGAGGTGAAGCCAGCTGGACTTCCTAGGTCAAGTGGGGACATGGAGAACTTTTCTGTC
TAGCTAGAGGATTGTAAACGCACCAGTCGGTGCTCTGTATGTAGCTAAAGGATTGTAAACACACCAATCA
GCACTCTGTAAAAACAGACCAATGAGCACTCTATGTCTAGCTAAAGGATTGTAAATGCACCAATCAGCAC
TCTGTAAAAATGGACCAATCAGCACTCTGTAAAATGGACTAATCAGCAGGATGTGGGCGGGACCAAACAA
GGGAATAAAAGCTGGCCACCCCAGCCAGCAGCGGCCACCAGCTCACTTGGGTCGCCTTCCACACTGTGGA
AGCTTTGTTCTTTCGCTCTTCACAATAAGTCTTGCTGCTGCTCACTCTTTGGGTCCGCACTACCTTTATG
AGCTGTAACACTCACTGCGAAGGTCTGCGGCTTCACTCCTGAAGTCAGAGAGACCACGAACCCACCATAA
GGAAGAAACTCCAGACACATCTGAACATCTGAAGGAACAAACTCTGGACACACCATCTTTAAGAACTGTA
ACACTCACTGCGAGAGTCCGCGGCTTCATTCTTGAAGTCAGCGAGACCGAGAACCCACAAGAAGGAATAA
ATTCCGGACACAATAAGGTTAAATAATTTTGCATTGGAAACTGGTTCTTTATTCATCCATGGGCCAGATG
CAGCATAGAAAGCAAGGTATGTGCTTCCAAAGATATCTCACCCCTGAAAGGAACCTTGAGCTCTCGAAAC
CACTCACTGGTATGAGGAACAGTTAGATTGCACTCCAGGTGGTGGTGTGACTGCACTGCAGCCGCACTAC
ACCGCACGGAACCCTGGCTCCTCAGACAGACCTCTTTCACTAGGCTGGAACTGCTGGGGACGGCACAGGG
CTAAGTCTTCATTGTGGCTTTAAAGAAATACCTGCATTGATTTCTGAATGGCAAACTTAAAAAAATTTTT
TTTTTACTGAAATAATTAAGGAAGCAACCATTTAAAGAGGATTTGTCAGCTTTTCAGACATTATATTCCG
CAGAGTTGGGCCATGTCTTTTTGTTCATGTTCTTTTTTGTTTTGTGTTGTAACTCATAGCAGGCACAAAA
TAAAAAACTACTGAATGGATAAAATTATTTAAAATTTAATAATTCAGAATTTTTTCCCTTCTCTTTTTCT
AAAAGGTAAGACTACCCAGAATTTTGTTTTATTTTGAACTGATCTGTTACAAGATCACTGGAAAGAGCCA
TGAAAGAATTACAATGTGTGTGTGTGTGTGTGTGTGTGTGTGTGTGTGTACATGTGTGCACACAAGCACT
GACAGGTCCTAAGTCCACATCTCCAGGTCGAACCTTTCCCCTGAGCCCCACATTTTCAGCTGCCTGTGTG
ACATCTCCACTTGGGTGACCATGAGTATCTCAAACTAAACATGTTCCAGATTAATTCCTGATCTTTTCCC
CAAAACTTTATTCTCCCTCCAGGCTTCCTTAACTCAGTTAAAAAGCAGCTTCATTCTTCCTGTTTCCTCA
GGTCAAAAGCCCTGGCATCATCCTTGGCTGCTCTTACTCTCCCATCTCACCTCAAGTCTATCAATCCATC
CAGTCTGTTGATTTTATCTGTGACACGTGTCCAGAATCTGACCACTTCCTACCAGCCTCACTGCTTCTAC
CCCCATCCACGCCACTGTTGCCCTCTCCAGGAACATTGCAGTATCTTTGTGTGTGCTCTCCTAGAGTCTC
AACCAGAAGGATTTTTTTTTTTTTTTTTTTTTGAGAAGGAATCTCACCCTGTCGCTCAGGCTGAAGTGCA
GTGGTGTGATCTCAGCTCACTGCAACTTCCACCTCCCGTATTCAAGTGATTCTCCTGCCTCAGCCTCCCA
AGTAGCTGGGATTACAGGCGCCTGCCACCACACCCAGCTAATTTTTGCATTTTTAGTAGAAATGGGGTTT
TGCCATGTTGGCCAGGCTGGTCTTGAACTCCTGACCTCAGGTGATCCACCTGCCTTGGCCTTCCAAAGTG
TTGGGATTACAGGTGTGAGCCATCGTGCCCAGCCAAGAAGGATCTTTTTAAGTGTCTCTGTTCAGGAGCC
TCGGGTAGGTTCCTGTCTTATTCAAAGTGAAATGCAAAGTCCATGCTTGGCTCATGAGGCCCTCTGTGAC
CTGGCTGTGGGGTGTTACTGATTTTCTCTCATATTCTCACTTCTCTCAGCCACAGTGGCCTCCTCACTAT
TCCCCATGTGTGCCCAGCAACTCCTTCCTGGGGTTTGGGGGTTTAAGCATCTGCCTTCACTGCTCCTCTC
CGAGAACAACACTGCTCCTCTGGGTACCATTTGGGTCACTGCTGGATGTCACCTCCTCATGGAGGCCTGC
CTCATCATCCTGGGCTAGACCCTCACTCTCTTCGCTTGCACGCGGTCTTAGTGCTCATCAGCTCCTGACA
TGCTGGGTTTTTTTCTGTCTCCCCATTACCTCCCAACTAAGTTACATGTTCCATGAGTGCAGAGACCTTC
TTGTACAGGCTAGGATAGTGCCTGGCACACAGTAAGCGTTCAGTGATTATTTGCTGAATCAGTAAAGAAA
TGAATAGGAATTTTTGTGGCAAAATGTGGTGGGATGAGGAGAGGGACTTCCACATGGAGAGGAGAGAATG
GACAAAGTTGACCATGGCCAAGAAATAGTGATCACTCCAGTTAGGCTGGAGAATGAGGTACCTAGAGGGA
GAAGTTAAGGTTAGAAAAATAGTTTGGGACGCATTTTGGAGGACTGTGAGTTTGTTTATAAATTAATGGT
GTGAGGCAATAGTGCTGCGAGAGTGTTGTAATGTGGCACCTGTCATGCTCCGCATGCCCCTGCTTCCGCC
CTGGCTCCCCTCCCATCTTTTCTTCATCCTCCATCCCTCATTTATTCCACAATTGTATCCTGAGTGCCTA
CTGTGTACAAGCAGGTGTTGGGGATGGAAAGGTCTTGCCTTTGAGGATCTGGTGATTTTATTGCATTTTA
CCAAGTGAAGGGGTGGAACAGCATGCAGAAGGCGCTGAGGTAGCCAGAGGGAAGGGAGAGACACTTTGTT
GGAGGAGTGCAGAGAGGTTGCAGGGGTGGTTAAGAGGCTTCCAGAGGGGAACAGTTGAGTTTGCAACATG
AAGAACAAGGCCCCAAGTGAGCAGAGGCTGAGGGCCTGTATGGTACTAGGAGACCTGAAAGTGATTTGAG
GTGTAGGCAGAGAGATAGCTAGAAACGTTCTTCTTGCTAATGATAAGAAGAATGGCCATCTCATTAATCA
AATGTTAGGTACTCAAGAAGGTTTAGCTGGGCCTGATCCCAATGGGCCACTTGGCCCGTAGTAAAGAATT
CACTTAACTTGGAAGGCAAAACTTTTAATTGAAAGGGTTTAAATGGGGGCTGGGGGACACATATGTAAAT
ATAAATCAGATGGTGGCTTCTTTGTTGAAAATACGTTCAGCACAGTGCAGGAGGGCAGGAATTGGCAGGT
TGAGATGTTGGAGAAGTGGGAGTGGAGCAGTGGAGGATAGCGTTAGGTTTTAGGAGACTAATAACACTTC
TGTCAGTCGTCTCCTGTCTTAGGAACTAGTTGAGAATAAGATACCAGAGGCAGGCAATTGGACAGTTCCT
TTACACCTAAACAGATTCAAGAATGTAGCTTGTGTCTGTGGTTTGGTTGGCTCCTCTGGATTTAGTAGAT
AAATCCACCAGGCACTGGTGAGCTGAGCCCCCAGAGGCCTACCTGGCTGGGACTGAGCCTGCCTGGCCCA
GTATGGTGCAGCAGTAGGCCAGGACGCATCCTTTCTGCAGGCAAGGCAAGTTGGAAAGACAAAGGAAAAA
GGACCTATGGCGGGCAGGGCATTTTAAATTTTCTCTCCCGAACTGACCAAGTAGATCATTTCTCTTAGAG
AGGAGAGAGAGTGGGTTGAGAGATGATGTCATAGGTCGGGCTCTCAGGAACTCTGACTTTGAGATGGAGA
TTAGTGTTCAGGGAGTGCTTTGGGGAGGAACAGCTATGGAGTAGAAAGGAAGGAGACAGGACTGTGCAGA
GGAAAAAGTTTAGCTGCACTGCAGTCTCCACAGAACCGTCAGCTGTGCCTGTGGGGGTAGAAGCGAGAGA
CCCCTCAGAGCTTTCCTGTGCTGGGACGGGAGGGCTGGACCTTTGTAGCTCTACATCCATCAGTCCTTGA
GATGTGGCCACTATGGGATGGGGGTGTGACCTTGGGCAAAGCAGAGACAGCTGAATATGATATGGTTGTC
AAATCACAACATCCTTCACAGATATTGCACTTGTTCCTCCCCTGCTTTGCCCTCCTGGGGAGTGGGTGGA
CATTCTGTCTTTGGGAACCTGGGCAGTTTATTCTGTCTTCTCTGGGAACCCGGGCAGTTTGTCTTCCTCT
AAGAAGGAATGCCGAGGTTTTGTGGTTGAAGATAGACCTTTCCCTGAGAAGTATAAGGGGAAGATGTGGT
GGGATCCATGACAGAAGTCACTAGAATGTGCGAAAGGGAGGAGCAGGAGACAGCGCATCCCAGCAGCTGC
CCTCACTCCTGTCTGCCGTGTTCCTGGGCAGCTACCCTCACTCCTGTCTGCCGTGTTCCTGGGCAGCTGC
CCGGGGAGACGCTGTCTTCTCCTCTGCCTTCCTCACTGCCTTTTGGTGTCCTTTTCCATCCCTAGAGCAT
AGATCAGGGACTACAATAAAGTTGAATCCTTTTCCTTTTCACCCCCTGTCTATTATTGAATAAAAGTGGC
TAGTAACAGGCATCTGAAAAAGAGAAATTGTGGAAAATTTTTTTATACTTTTGTAAGTGTATTTTTCCTG
GAGGTTTGGAAACCACAAAAGTTTTCTGTCATACTTTATTAGGGTAGCAGAATTGGTGGTAGGTTCTAAA
TTCAGCAAGTACATTTAGTAAATCCACAAAATGCCATTGCTTTCTTAAATATCATGGTTTTATTTAGTCT
GAAAATTTTTTCTCAGCATTTCATAGTTTTGCAGTGTAAATATGGCATTTAGCATCATTTTACAGGGCTC
TGGAAAATGTGGAAATTACTGTTATTCCCATTTTCAGTATTTCAAGTCCATTTGTTGTGATCAGTACTTC
ATTCCACACATAAGGTCTAATTGTGAATTTTTAAAATGTCTCTATATGTGCATGTTTTGTGGGAGTGGTA
GCTGTCTCCTGAGTCATTAATGTGAGTGCACTGAACTTCTTAAACTTCCCATTATGAAAATTTGCCTCCC
CTAAAGGGGTAAAATGAGATACCATTTGTATTCCTAGGTAGGTTTTAATTTTGTTTGGAAGTTTTGTTTT
ATTTTGTTTTTTTCATTTTATTTACATGGAGTTCTGCTTATTATAATTCTAATTGTATCAGATTGTTTCT
TAGTGATTTGCCAGTGCTCAGATGGGCCAGATCAAGACATCTATTGCCAATTAGGAACAGGATGTTTATG
GTATTTACTGATGTTCTCCGGATGACCTGTTTTATGTTTTCAGTTTGTTTTGTTTTATGATCATGTTGGC
TTTTCCATTAAACTATAACTTTAACTGAGTTGGCTTAGTTAGTAAAACATTGACCAGCTCCCCTTCTCTA
TCCTCCAGGCCCCTTCTGATGCCTCTTCCCCTATTGTGCCCCAGCCTCAGTCTGAAGGGAAGCGAATTTT
TAGGTCTCTATGTTCCAGATGTAATCTATATTTCCAAGACATAAGCTCACTGTATTTCAACCTAAATTGC
CTGAGTAATGCTCCTTGGAACATTTAGGGATAAATCCCTGTGAAAAGTAAATTTAAACAGTGACGTCTGT
AGTGGGAGGTATTTCTTAATGAATAGCCACTAGTGAGGATGTGTGGATGTCTGCACTACACGTGGCTGCC
TGATCGCTACATCTGTTTTGAAGTTCAGTGATTTCCCAGACCACATGTTCATTTCTTTAGGGGGCTGACA
TCCCTCCCAGGATCCAGTCATTGTGAGCTGGTAACTGTAACTGGCTTACTCTTTTTGACCTAGAGATTAT
TATACTTTGTCTTAGTTTGACTATTGTTCCTATCAGATCTGACAATATTGGAGTTTCTATAAATTAGCAG
CATGGCAGAGGAACCAAGTGTATTTAAACCCATAAATCATGATTTCCAGGTCTTTTAAAAGCTGTGTGGT
TTGAATAGTACTTTACTCTATGGTAGACATAAAGAGTGGTTATAAAAATTCTGGCTAATCAGGAGGCTTT
TGTAGTTCAGCTGAAACTACAAAATAACCTCAGATGCGTCACTCCTTTGGGAATCAGACCATCCAGATTT
GTCCTGGCTCTGCCAGTGAGATCTTTTACAAATTACTTACCCTCATTCCTATATATTAAAAAAAAATAAC
AAATACTATATGATTCCACTTAAATAAGGCACCTAAAGTAGTCAAGTTTATAGAAACAGAAAGTAGAACA
AGCTGGGGAGCGGGGGTGATAGGGGAGTTGTTTAATGGATGCAGAGTCTAGGTTTTGCAAGATGAAAAAT
TTCAGGAGATCTGTTGCACAACAGTGTGAATGATTAAGATGATAAATTTTCTGTTTTTTTTTTTTAACCA
ATCTTCAAAAGGATGGTATTACTGTTAACTGAATGCTTGGTATGTGTTATGCATGCATTATCTTGTTAAC
TATTCACAACTCCATGAAGGTAGATATTGTCACCGTGTTTATAGGTGAGAGAACTTAAATGGAGTTTACT
AGCTAGGAAGTGGCAGACCATGTTGGGCCTAGATGTGTCCGTAAAGCCACTTGAAAAATGTGTTGCCACC
AGCACACTTAAAGGCAGGCTCACTCTCGGGCTTTTTAGCTCTAGAATCTGTGATTCTATGATTCTGAGAC
ACGCCAGGGACTTTGCAAAGACCCATCAAGTAGGAACAAAGTTTAAATGTTTATGTGTTCAGAGACCACT
TCTCTACTATTTTTAGTGAATACAAACTGCCATATTATATGTAATCTGTGGTACATCAGCAGCACCAAAT
GCTGGTTTGTGAGAGGGTGAGATGTTACTGAAATGTGAGAAAAGTTAAGGTGTAAATAGTAATTACTTCT
TTTTTTTGTTCGTTTGTCTGTTTGTTTTCCTTGAGACGGAGTTTCACTCTTGTTGCCCAGGCTGGAGTGC
AACGGCACGATCTTGGCTCACCACCACTTCTGCCTCCCGGGTTCAAGCAATTCTCATGCCTCAGCCTCCC
GAGTAGCTGGGATTACAGGCATGTGCCACCACACCTGGGTAATTTTTGTATTGTCAGTAGAGACGGGGTT
TCTCCATGTTGGTCAGGCTGGTCTCGAACTCCCGACCTCAGGTGATCCACCCGCCTTGGCCTCCCAAAGT
GCTGGGATTACAGGTGTGAGCCACTGTACCCTGCTAATAGTAATTACTTCTATGGAATGGTTTGGTGAAG
ACTGAGAAGAACCTCACCTATTCATTATATTTTTCGACTTTTTGAGTTTGTAATAATTAGCTTGTATTAC
TTATGTTATAGGTTGAATTTGTGCCCCCAAATTCATATGTTGAAGTACTAACCCCCAATACCTCAGAATG
TGGCCTTACTTGGAAACAGGGAATTGCAGAAGCGATTAGTTAAGATGAAGTCATACTGGATTAAGGTGGA
CCCTAATCAACATGACTCTTGTCTTTATAAAGAGGGGAAATTTAGACGTAGACATGCACACAGGGAGAAT
GCTATGTGGAGATGAAGAAAGAGCTCAGCTGATGCTTCTGCAAACCAAGGAATACCAAAGACTGCCAGGA
AGCCCCAGAAGTGTAGGGAGAGGCATTTCCTCACAGCCCTCACAAGGTATCAGCCCTTAGGACACCTTGC
TGTCAGAATTCTAGTATCAAGACTGTGAGACAAATTTCTGTGGTTTAAGCCACTCAACTTGTGGTACTTT
GTCACAGCAGCTCTAGCAAACTAGTACAATTTCTAAAATAAAAAGGTTTTTATTACATACAAAAAAATGT
TAACCATGGTTACTGATAGTTTATCCAAAGCAGTGTTGAAATTAAGAAATGATCTTTATATGTGAAGATG
TTTATTGTAATATTTTTTATAATCAGAAAAAATGAGAATCCAAATATCTAGGCATTGGTGAATAACCAGG
TATAACAAATTATAGCATAACTACTTGATGAAATATTGTATGGTCATTTTGTATAGCCTTTGAAATGTGA
AAAATGCATATAACATAAATAAGCTGGATGAAAATTTTTTATACAAAATGATAACAGTTGTATGAAATAT
ATATGCATCAGACTGAAATTGGAAGGAAACACAGAAAATGAAAAAGCTTTGTTAGGATTATGGAATCATA
AGTGGGCTTTTTTCTTTTAATCTTTTAATTTGTTGCTTTTATTACATAGAATTGTGGCAATGTATTATTG
TGTTATATTATTTTTTAAAAATCAACTTTATTGAGGAACATTTTACGTGCGATCAAATGCACTCTTAAAG
TGTATAATTTGATAGTTTTGACACATGTATACCTCTTTATAACCACCGCCACAATCAAGAAACAAAACAT
GTTCATCACCCCAGAAAGTTCTCTCTTGCCCTTTTATAGTCAGTCCTTCTCCCAACTCTGGTCTCAGTTA
GCCACTGATCTGCTTTCTGTCACCATAGATGAACTTTTTCTTTTCTATAATTTCATATAAACATAATAAT
ATAGTATATACTCTGTGTCTGGCATCCTTCATTCGGCATAATGTTTCTGAGATTTATCCATGTTCTAGCT
TATATGTAGCTGATTACTTTTTATTGTTCAGAATGTTCCAATGGTATGTTGTATCCATATACCAGTTTGT
TTATCTGTTCATTTGTTGATAGCAATTTGGATTGTTTCTAGTTTTTGGCTATGAGTAAGTTGCTATGAAC
AATTTTTTTTTTTTTTTTTTTTTTTTTGAGATGGGGCCTTGCTCTGCTGCTCAGGCTGGACTTAAACATC
TGGGCTCAAGTAATGCTCTCACCTCAGCCTCCCAGGTAGCTGGGACTATAGACATGTGCTCCTAGGCCCA
GTTTGAACATTTTTTGTAAAAGTTTTATATAGTCATACATTTTCATTTCCCTTGGATAAATGCCTAGGAT
TGGAATTGCTGGATTGTGTGATAGTATGTGTTTAATATTATAAGAAACTGCCAAATGGTTTACATTCTCA
CCACCAACCTATGAGAGTTCCAGTAGCTGTACATGTTCACCAACACCTCACATTATCAGTCTGTTTTTAT
CTTAGTCGTCCTCATGGATGTATAGCAGTCTCTCAAAGTGGTTTTGGTATGAATTTCCCTGATGATTTGA
CACTGATCATCTTTTCATGTGATTCTATCTTCTTTTGTGAAGTGTCTATTAAAATATTTTGCCCATTAAA
AAATATTTATCTTCTTCTTAAGGTGTGTAAGACTACCCTTTATATTCCAGATACTGTTCCTTTGTCAAAG
ATATGTATTCAGAGAACATATCCACTCAGTTGTGGCTTGCCTTTTCATTTTCTTAATTGTATACTCAAAA
GTGCATATGTTTTAAATTTTTACTTTAAATTTAAAAATTTTCCTGATTTGTCATTTTGTTCTGTTATAAT
TCATGCATTTTTTTCTATTATAATTCATGCTTTTTATACCTAAAAAATTTTTGCCTATGTCAAGTTTATG
AAGATTTTCTCCTGTGTTTTCTTCCAAAAGTTTTATGTGTTACTTTTTACATTTAGATATGTGATCTATT
TTAAGTTAACTTTTCTAGACAGTTATGAGGTGGGAGTAGAGGGTCTAGATTTCCATTTGTTCTCTGTTTT
CTTGTTTTAGCTCGTTGAACATAATTTCAGACAGATTTTGTGTGTGGCTTTTTGTTTTTGTTTTTGTTTT
TTTCAGATGCTCTGTCGCCAGGCTGGAGTGCAGTGGCGCAATCTCAGCTCCCGGGTTCAAGCGATTCCCC
TGCCTCAGCCTTCCGGAGTAGCTGGGACTACAGGTGCATGCCACCATGCCTGGCTAATTTTTTGTATTTT
TTAGTAGAAACGGGATTTCACCATGTTGGCCAGGGTGGTCTCGATCTCTTGACCTCGTGATCTGCCCGCC
TCAGCCTCCCAAAGTGCTGGGATTACAGGAATGAGCCACCACGCCCGGCCCAGACAGTTTTTTAAAAGTC
TTTGTCTAGTAAGTTCAAACCTGTATTTCTTTTATTTATTTATTTATTTATTATTATTTTTTATTATGCC
TTAAGTTCTAGGGTACATGTGCACAGCGTGCAGGTTTGTTACATATGTATACATGTGCCATGTTGGTGTG
CTGCACCCATTAACTCGTCATTTACATTAGGTATTTCTCCTAATGCTATCCCTCCCCTCTCCCCCTACCC
CATGACAGGCCCCGGTGTGTGATGTTCCCCACCCTGTGTCCAAGTGTTCTCATTGTTCAATTCCCACCTA
TGAGTGAGAACATGCGATGTTTGGTTTTCTGTCCTTGTGGTAGCTTGCTCAGAATGATGGTTTCCAGCTT
CATCCATGTCCCTACAAAGGACATGAACACATCCTTTTGTATGGCTGCATAGTATTCCATGCTGTATATG
TGCCACATTTTCTTAATCCAGTCTATCATTGTTGGACATTTGGGTTGGTTCCAAGTCTTTGCTATTGTGA
ATAGTGCTGCGGTAAACATACGTGTGCATGTGTCTTTATAACAGCATGATTTATAATCCTTTGGGTGTAT
ACCCAGTAATGGGATGGCTAGGTCAAATGGTATTTCTAGTTCTAGATCCTTGAGGAATTGCCATACTATC
TTCCACAATGGTTGAACTTGTTTATACTCCCACCAACAGTGTAAAAGTGTTCCTATTTCTCCACATCCTC
TCCAGCACCTGTTGTTTCCTGACTTTTTAATGATTGCCATTCTAACTGGTGTGAGATGGTATCTCATTGT
GGTTTTGATTTGCATTTCTCTGATGACCAGTGATGACGAGCATTTTTTCCTGTGTCTGTTGGCTGCATAA
ATGTGTTCTTTTGAGAAGTGTCTATTCATATCCTTTGCCCACTTTTTGATGGGGTTGTTTGATTTTTTTC
TTGTAAATTTGTTTAAGTTCTTTGTAGATTCTGGATATTAGCCCTTTGTCAGATGGCTAGATTGCAAAAA
TTTTCTCCCATTCTGTAGGTTGCCTGTTCACTCTGATGGTAGTTTCTTTTGCTTGTGCACAAGCTCTTTA
GTTTAATTAGATCCCATTTGTCTATTTTGGCTTTTGTTGCCATTGCTTTTGGTGTTTTAGTCATGAAGTC
CTTGTCCATGCCTAGATCCTGAATGGTACGCCTAGGTTTTCTTCTAGGGTTTTTATGGTTTTAGGTCTAA
CATTTAAGCTTTTAATACATCTTGAATTAATTTTTGTATAAGGTGTAAGGGAGGAATCCAGTTTCAGCTT
TCTACATATGGCTAGCCAGTTTTCCCAGCACCATTTATTGAATAGGGAATCCTTTCCCCATTTCTTGTTT
TTGTCAGGTCAAACCTGTATTTCTTTAGGGTTGTTTCTAGAAATTTATATCGTTCCTTTGAAGGGGCCAT
GTTTCCCTGTGTCTTTGTGTGTCTTGTGATATTTTGTTGACAACTGGTGATTTGAAAAAAAACAGCCAAT
TCTCTCAATTTTTGCAGACTGGCTCCATGTGGGGAAAGACCTTCAGTAGTTAGCCCAGTGTAAAGGCCTA
AGGTCTTACACTTTTTTGGCATATACATCTTCCTTGGATTTGTCTGTCTGGTTTTTTGTTTTTTTCCAAT
TCCGCTGTATACATGGCTGCTGTTACATGTATTAATTTCTCTAAGAGCCTTACTCCTGCTGCTTCACAGA
GACTCAGCTGTTCTGCTGTTTTTCTCTGCCCATAATCTCTTGCCCACAGGTGACTGCAGTTTCCTGCAGT
TTTCATGCACTACAGAACTTGCCACTACCTTCTGCAGCCTTCAACCTGAGATCCAAAGTGTCCCAGCATT
CCCATCCGAACTCTGAGTCAGGCAATGAAGAACCAGTCCCTTGGGCAGCCCGCAGACATACTTTTCTCTG
TAATGAGGGAGAAAACTAGGAATTGGGTCTCTTTGTCTCTCCCATGCTATGCCAGGGAAAGGCTAGGGCA
AGTGCAAGCAAGCACAGCATGACATTTCCTATCGTTTTCAGTGTGGCTTTTTCTTGATTGGGTGTTTGCT
TGGTTGCTGCATATCCTTGACCTGTTTCCACAGCTTCCACAAAACCATTTTAATCAGCATGTAGTTGTTT
GTTTGATGTTTCCATGGATGAACGGAGAGGGCCATCCTAGTCTGCCATCTAGCTAACATCACCCTATCAA
ATGCTGACTATTTTTAAATGTTTTTTCTGCATCTATTGAGATGATCATATAGTTTTTCTTCTTTATACTA
TTCTAGTATGGAGAATGCAGTCACTGATTTTTAATGTTAAACCTACTTTTCATTACTGGGATGGTCCCCA
TTTAGTCATGTAATGTTATCTTTTTAATATATTGCTGCTATATGCTAAAATGTTAAGGGTTTTTATGTCT
ATGTTCATGAGAAATATTGATCTGTTGTTTTCTTACAGTGTCTTTGTTTCGACATCAGGGTAATTCACTC
TTATTATTATTTTTTTGAGACAGGGTCTCCTTCTGTTGCGCAGGCTGGAGTGCAGTGGCCAATCATAGCT
CACTGCAGCCTTGACCTCCTGGGCTCAAGCAGTCCTCCCACCTCAGCCTACTGTGTAGCTAGGACTACAG
GCATGTACCTCTGCAGCTGGCTAATTTTGAAATTTTTTATGGCAACGAGGTTTTCCTGTGTTGCCCAGGC
TAGTCTCGAACTCCTAGTCTCAAGCGATTCTCCCACCTTAGCCTCCCAAAGTGCTAGGATAACAGGCATG
GGCCACCACACTCGGCCTGCCCTTATTTTTCTTCCTTAAATGTTTAATTGAAGTCACTAGTGAAACTTTA
TGGTCCTCAGATTTTCTTTGGGGGAAGTTTTTAAATTGCAGGTTCCTTATTTTTAAAATAGGTAGTTTCT
ATTCAGGTTCTCTGTTTCTTCTTGAGTCGGTTTTAATAATTTTTGTCATTTAAAGAATTACCTATTCCAT
CTTTGTTGTCAAGTTTATTGATATAACATCCTTTAATAATGCTTTTAGTATCTGTAGGATATGTAATGAT
GTCTTCTCTTTTATTTCTGACATTGGAATTTGTGTCTTCTCCCTTTTTTCTCCCCCATGAACTGAGCAAC
GGTTTAACAAATTAAATTTTTTTAAATAGCCAGGTTTTGGGTTTATTGATTTTATCTATTGTTTGTGATG
TCTTTGATTTTTGCTCTTTATGATTTTCTTTCTTCTACTTACTTTATATTTAATTTGATCTTTCTTACCC
TAGCTCTTAAGCTGGTGACAAATACTTGCTCAGAGACCTTTCTTTATTTACACCATGAGACTTAAAGCTA
TAAATTTCCCTCAGTCGCTGCTTTATGTGCATCCACTGAATTTTGATACATTGTGTTTTTATTTAAATTT
AGTTAAAAAAAATTTACTAATTTTCCTTGTGATTTCTTTTTTTGACCATTGGTTATTTAGAAATGTGTTT
TTTGTTTGTTTTTTTTTTGAGATGGAGTCTCACCGTCACCCAGGCTGGAGTGCAGTGGCACAATCTGGCT
CACTGCAACCTCTGCCTCCTGGGTTCATGCCATTCTCCTGCCTCAGCCTCCCGAGTAGCTGGGACCACAG
GCGCCCACCACCACACCCGGCTAATGTTTTGTATTTTTGGTAGAGATGGTGTTTCACCATGTTACGCAGG
ATGGTCTCGATCTCCTGACCTCATGATCCACCCACCTCGGCCTCCCAAAGTGCTGGGATTACAGGTGTGA
GCCCCCAGGCCCGGCCTAGAAATGTGTTTTTAAATTAACAAATATTTGAGGCTTTTCCACATATCTTTCT
GTTACTGATGTATAATTTAATTTTGTTGTGTCTGATTATATATTTGTATTATTTCAGTCTTCTCAAATTC
ACTGAGAATTGTTTTATGGTTCTGAATTTGGTTTGTTTCAGTAATTGTTCCATGTAAACTTCAAAATAAT
GTTGGGTGGAATATTTTCTAAATGTCAGTTGGGTTAAGATGGTTGATAATTATCGAAGACTGTGGCCGGT
CTGAGATTTTTTTTTCCTTCCTTTTTGGAAGGTAACAAGTTAGACTGCCACTGTTTCATGCATGCTGGCT
GAAGACATAGGACTTCTGGGTCAGAGACAAAGGATTTTATAATCACAGCACAGCAAGCCACAAGAGTTTG
GTGTTTGTATCTGCTCCTCTGGTCCCAGAAGACCTATGGGAGAGATGCAGAGAGTCCTAGATAAATACTG
TGCACATGTGGGTTTGTGTCACAGCTGAGGAACCTACAGCTTGTATTGCTCAAATCTTTTATAATAGGCT
GCAAACAAACTTCCTTTTTCGTGGAGGAAGACATTATTCTTATTGTTGGACAGTTAGCAAACCTGTCCCA
TTTCTTCTGAATGAGACCCTGTCTCTTTTCCAAAGGGGTTGGCTGTACAAATATCATTGAAAATATAGTC
TGGAGCAAAAGCAGAAATGCAGCAGACCCATGGAGAATTGTCTACCAATAATAGTGTTGTTCAGGTCCCT
ATAGTCTTCTGATTTTCTGTCTATTTTTATCAATCTCCTAAGAGATGATGTTGAAGCCTCCAATTATGAG
TTATTAATTGCTATTTTCAATTCTGTCATTTTTTGTTTCATTTATTTCTAAGCTCTGTAACTGTGTATAA
ACACATTTAGAATTGTTACATCTTCTTGATGAATTGACCCTTTTATCATTGTGAAATCTTTCTTTTTTCC
TGGTAATACTTTTTGTTTTTAAGTCTGCTTTGTCTGACATTAAAACAACAGTAACAACAATAACAGTTTT
CTTTTGATTACTGTTTGCATGGTACATCCTTTTACTTTTAACTTACTTGTGTCTTTAAAGTGGGTTTTTC
ATAGGTAGCGTATAGTTTTTGGTTTTATCTAATGTGACACCTGCTGCTTTTTCATCTAATATTTAAAACA
TTTCTATGTAATATAATTATTGATATGGCTGGTTTAAATCTTTAATAATAGTTGCTGTTGTTAAGTAACA
AGCCATTCCAAAGTTAGTGAATAAAATAATAATCCTTTTAATGCCTTTTTAGATTCTGGGTTAGGAACTC
CACTACACAATGTAATTGGCCTCCACTGGGAAGACTTGAATGCTGGCAGGATTCAGCATCTGGGGCTGGA
GTCACCTCCGTGCTTACTCCCTAGTTTTCCTGACGTTAGTTGATACCGACTGTCAGCTGGTTCCTCAAAT
GAGGCTGTCATCTGGAACCCATGGCCTCTACGTGGGGCTTCTTGGCTTCCTAACAGCATAATGGCTGGGT
TTCATGAGAGTGTCCCCAAGAGAACCAGGCAGAAGCTACATAGCCTTTTGTAACCTATCCTCAGAAATCA
CAGAGTTATACTGTACTCTCTTAGCAGGAGCAGTCATACTACCCCACTCAGATTCAAGGGAAGGGATCTG
AAACCCCACCTTTCGGGGGGAGGAGTATCAAAGATCTTTAACACCAGACTTCAAAACTGCCACATCAAGC
ACCTTGCTATTTGTTTTCTATTTTTGTCATCTGTTCTTTGTTCTTTCCTCCTCTTTCCCATCCTCCTCAC
TCTCTCTCCCTATCTCCCTCTCTCCATCTCCCTCTCCTCCTCTCCTACCACCTTCTTCCCTCACTTTCTT
GTTTCTCCTTACCTCTTCAGAGACCGTATAAACTGCTCTCTGCTCCCCTAAGAGAGGCTCTTGTTTTTAG
GAATTTAATTCTTTTACATTTCTTTGCATCCTCAGATGCAAAGTGGTTTACCCATTTTATCTAGTTATTA
CTGCAGAAGCAGTGATCTGTATCCGAACTGTAAGAGATATCTTTGTCTTTGAACTGGTAAGTTTACACCA
TTTAATTTTTTAAAATTACTGCTAGTTTGGAACTCTTTTTTATAACTAAATTTATTACTTCAATTTGACC
TATTTTTAATGTTTCTTTTAAACTTTTTATCATATATTTTTGTGGTAAAATGCATATAAAATTTATTATC
TTAACCATTTTTAAGTATATAGTTCAGTGGTATTAAGTACATTCATATTGTTATGCAGCTATTACTACCA
TCCATCCTCAGAACTCTTTTCATATTGTGAAACAGTACCCATTCAACAGTAATTCTCCATTTCCTCTGCC
CCGGCACCACCATTCTACATTCTCTCTCTGTGATTTTTGACTACTCTAAGTACCTCATATAAGCGGAGTC
ATACGTTTGTCTTTCTGTGTCTGACTTACTCCACTAAGCATAATGCCATCAAGGTTCATCCATGTTCTAA
CGTAAGTCAGAATTTCCTTCCTTTTTAGGACTGAGTAAAATTCCATTGCATGAAGAGACTGCATTTTGCT
TATCCATTCGTCTGTCAGTGGACACTTGGGTTGCCTCTACAGTTTAGTTGTTTGTGGATAATGCTGGTGG
GAACATGGGTGTACAAATATCTCTTCAGGATCCTGCTTTCAGTTCTTTTAAGTATAAACCCAGAAGTGGA
ATTGCTGGATCATCTGGTAATTCTATTTTTAATTTTTTATATATTATTATTATTTTTAGAGACAAGGTCT
TGCTGTGTTTCCCAGGCTAGAGTGCAGTAATGTGATCATGGCTCACTGTAGTCTCAAACTCCTGGGCTCA
ACCGATCTTCCAACCCCAGCCTCTTCAGTAGCTGGGACTACAGGCATGCACCATCATGCTCAGCTAATTT
TCCAAATATGTTTTTTGTAGAAATAGAGTCTTGCTTTGTTCCCCAGGCTGTTCTCAACTCATGGCTTTAA
GCAGTCCTCCTGCCTCGGCCTCCCACAGTGCTGGGGTTACCAGTGTGAGCCACCACACCTGGCCCATTTT
CTGTTTTTTTCATAGTAGCTATCTTAATGGGTGTAACATAGTATCACATTGTAATAGTTTTGAGTTGCAT
TTTTCTAATGATTAATGGTGTTGAACATCTTTTCATGTGCTTGTTAGCTATTTGTATGTCATGTTTGGAG
AAATGCCTGTTCAGTCCATCTGTTTTTCAGTTGTGTTGTTTGGTTTTTATTGTTGAGTTTTAGTTCTTTA
TACATTCCAGGTATTAATCCCTTATCCTGGTATATGATTTGCAAATATTTTCTCCCATTCTGTGGGTTGC
CTATTTACTGTATCTGTAGTGTCTTTTGAAAGTTTTAAATTTCGACTATTTGATTTGCCTGTTTTTTTCT
TTTGTTGTCCGTGCCTTTGGTGTCACATCCAAGAAATCATTGCCAAATCTGTTGTCATAAAGCTTTGCCC
TATCTTTTCTTCTAAGAATTTTATAGTTTTAGGTCTTAAATGTAAGCCATGGATGCATTTTGAGTTAATA
TTTGTATATGGGGCTAGGTAAGGGTACACAGTTGCATTATTTTCCATATGGATAGTCAGAATTTCCCACC
ATCATTTGTTGCAAAGGCTGTCTTTTCCCCATTGAATGATCTTGGCACCCTTGTTGAAAATCATTCAACC
AGATATGCAGGGATTTATTATTGGACTCTGTATTCTATTCTACTGATCTGTATGTCTGTCTTTCTGCCTG
GACCACACTGTTTTTTATTACTGTAGCTTTTTAGTAAGTTTTGAAATCAGGAAGTGTGAGCCTTCTAGCT
TAGTTCTTCTTAGGGACTGTTTTGGCTATTCTAACTCTTTTGATTGATGGTTTTTACTGTTTCCTTTCAT
CTCTACTTTTTGCTTATTAGGCATATCTTTTTATCTCTTACTGGCTGCTCTAGGGCAGCAGTGTCCTATA
GAAATATAATGTGAGCCACAATTGTGACCCCCATATTTCCCATTAGCAACATTGAAAAACTCAAAAGAAA
GAAATGAAATTAATTATAAAAATATTTTATTTAACCCAGTATAATGAAAATATCATTGCAACATGTAGTC
GAAATAAACAGAATGAGATACTTTGTTCTCCTTCTCATACTAAATCTTTGAAATTCAGTGTGTAACTTGT
AGCTTATCTCAGTTCAGACAAGCCACATTTGAAGCTTTCATTTGCCCATGTGGTCAGTGGCTACTGTGTT
GGGAAGCACAGCTCTAGGATTTATAATATTCATATTTTGCATAATACATGACTACAAATATTACTTGAAA
ATAGAGTGCATAACATATGTGTATACTATTAGAACCTCATCACAGTCTACAGCCACATCTCCTCTCCCTT
CTGTTTATCGTGGTGGTACATTTACTTCTAAACATTTTATAAACCCACAGTATATTATTAGTTTTAAACA
AAATTACTGTTTTAAGAACATTGACAAGTAAGGAAAAAATCTTTTCTGTTTACCCACAGATTCTCCCTTT
TCAGCACTCTCCCTTTCTTCCTGCAGATACACATTTCCATGTGATGTCACTTTCCTTCCTCTTGAGTTTC
TCTTACCATTGCTTGGAGTGCATGTCTGCTGCTGACAAATTTTCTCAGCTTTGGTTTCTCGGAAAGAGGC
CTTATTTCACCTTCATTTTTGCATGATAGATTTCTAATTCGACAGGTTTTTTTCTTTGCTATTTGGTACT
TTAAAGAGGTAATTCCATTCACCTCTGTCTTGCATAGTTTCTGAGAAGGCTGTTGTCATTTTTATCTTTG
TTCATCTGTATCTTTTTCTACTTAGGCTGCTTTTAAGAATTTTTTCTTTATCATAGATTTTCAGAAGATT
GATTATGATGTATCATGGTATGGTTTTCTTTGTGTTCATCCTAGCTGGAGTCTTTCGAGATTCCTCTATT
TGTGCATTTATAGTTTTCATCAGATTTGGAAAAAATTTAGTTATCATTCCTTTAAGTGCTTATTCTGTCC
TGTACCCCCTTTCCTAGGACTTGAATTCTATCAGTGTTAACCTACGTGAGATTTTCCCAAAGATCATTGA
GGTTCTTGTCATAATTTTCCCCATCTGTTTTCTCTCTCTGCTTCCTTTTGGATAGTTTTATTGCTATGTC
TTTAAGTTTCTAAAGATTTTTCTTTTAAAAAAAGGTTTAAAGAAAGAATGACTTTAAATCTTTAAGTCTT
TCTCCTGAAGTATTTAGTCTGCTTAATTTTGCTTTGTGTATTTTTTATTTCATATATTGTATTCTATATC
TGTAGAAGTTGTGACTGGTTCTGGTTTTTTTTTTTTTTCTTACCATTCATTTCTCTCCTGATTATGTTTT
TCTTTAAATACCTATAATACGTGTAATTGTTTAAGGTCTTTACTACTTCCATCATCTCTGCCATTTCCAG
CCTGCTTCCTTAAGCTGCTTTTCTTTTTGGTTATGGGTCATATTTTACTGCTTCTGAGCATACCTAGTAA
CTTTTTATTAGATGCCAGGTATTATGAACATTGCATTGATGAATGTGTTGATTATGTTGTCTTCTTTTAG
AGAGAGTGTTGTTTTATCCCGGCAGACATTTATGTTTTGTAGATTAGGTCTATCCTTTCCAAGCTTGTTT
GTAAACGTTGTTGGGGCAGGTCTATAGTGGCCTTTCTCCAGGGCTAATTTTATCTGCCCTTTCATATGCA
CAGATAATTAATTTTACCAAGGCCAAGTAGCATGTTAAGGTCAAGTAGCATAACAAGGTCAAGTAGTACC
CTACATATGCACAGATTATTATTCAGCCAAAAACTCAGAGCCCCCATGTTGATTTCTGGAGCCTTTTCTC
TATCTAGTTTCCTCTTGGCTCTCAAATTGTAGCTATCTTTGCCTTATCAGACTCCAGTGTTGGTCTCCTT
AACTCAGTGAAACTTCTGTGCTCTGCTCACGATGGCTCTCCAGCCCTGATTCAGAAAGTGCACCCAGGCA
GAAAGCCAGGCCAATCATAGGGATTCCCTCATTGGTTTTTCTGCTTTCAGGATCACAGTCTGGTGCTGCC
TGTTGTCAAGTGTCTGAAGATATTTTGTTCAGTTTTCTAGTTTTCTGTAGCAGGAGGATACATATAATAG
CTTGCTAGGGTTAGAAGTCCTGAAGACAACTGGAGTGCTTTAAAATCCTTTGAAAAAATTGGTAAGCCTT
CAAGAAATTTTTAAGTAGTGTTAGAGGTTAGTATATATATTAATATGCTACTTGACCTTGGTAAAATTAA
CATTTTTCAGGTTACAAAGATCTATTGTAACCTGTCCTATTAGTATATAGATAGAGTTTATGGAATATAT
ATGACATGTTTTCAGAGTCTCCATTCTGTAATAATTGCCATTTGTTAAGATTGTTAAGTTGAAACAAATT
GTTGGGCATCTAGATCAGATTTAAAAGGAGTTAAACCAAATTGAGACTGAATTTTGGAATGGCTATGCCA
AGTACTCTGCAACACAGCTAATTGCCTGGGTTCAAATCCTGGTGTGTCACTTACTACTTATTACCCTGGG
CAAGTCATATAACTTTTCGAAATTACAGTTTTCTATCTGTAGTGACAAAATAACTTTACCACCTCGTAGG
ATTGTTGGATTAAGTGATTATATATAGTGTGTATGTGTATGTGTGTGTGTAAAGCTTTTAGAACAATGCC
TGATGTTATGTGCTCAAAAAATGTTAATTATTGTCATTGTGATTGTCATTGTTAGGGAACTGAGTTTCTG
ATTAAGGTTTATTAAGCCTGAATTTTTTTTTTACCATTTTAATTGAGACTTCTCTTGCTTTTTTAACCAA
GTATATCATTTTATAATTTAAAAAATAATAAATAACACATATCTATCATCCAGTGTTAGGAATAGAATGT
TACTGATATGTTCAATATCATCCACATAACTGTCCCTGCTCACATATCCTATTCTCTCTTCCTATCTTGA
ATTTTCTATTTATTGTCACCTTGCTTTCTTAAAAATAGTTTTGCTATGTATTGATTTAGTCATAAATAAT
ATATATTAATTTTTCCTGTTTCTAAAATTTTATATAGATATAATGACATGTATTCTTCTGCCACCTGCTT
GTTTGCTTGATGGTGTATTTTTTTGAGATTTGTTCATGTTGGTGCATGTAGTCGTGTGCATTCATTTGTA
CCAGGGTAGAGTGTGTTTATTTCCAGGTTTTGCTGTTACAAACAGTAGAGCTCTGAATATTTCTGTGCAT
ATTTCCTGGTATACATGTGCAAGAGTTTCTCTGGGTATATGTCTGGATGCAGATATTTCAGGTTACAATC
TTTAGCAGGCTGCTCGCTGGAGCAGGCCAGAGTGGAAGTAGGTTATTGCAGTAGGCCAGGCAAGGGGTTA
TGAGGGCTTGAACCAGAGTGATTACCTTGGAATGGAGGGAAGCAGATGAATTGGAGATTTTGTCTGGAAA
TATAATCAATAGATATTGATGGTGTATTTTATAAAGGGGGCCGGATGAGAAAGAACATCAAGGAGACTCC
CAGGATTTTACATCAAGTTGCCATTTACTGAAATGAGAACTGCTCCCAACAGATAGACATATTTTATTTT
GTTTTAATTCATACATGGGCTTTTAAAAAGAATAAACTTTATTTTTTAAAGCAGTTTTAGGTTTGCAGCA
AAATTGAGTGGAAGGTACAGGTATTTCCCATATCTACCCATTCCTACACATGCACAGCCTCCCCTGTTAT
CAACAGTCCCCTCCAGAGGGATGCATTGGTCACAACTGATGAACCTGTCCTGACACATCGTTATCATTCA
GAGTCTGTAGTTGACATTAGGGCTCACTCTGGGTAGTGTACATTCTATGGGTTTGGGCAAATGTGCAATG
ACACATATCCACCATTACAGTATCATACAGCGTAGTTTCACTGCCCTAGAAATTATTCGTGCTCTCCCTG
TTCATTCCCCTCTTCTCCCTAATCCCTAGCAGCCAGTGATCTTTTTACTGTATCCACATTTTTTCCTTCC
CCAGAATGTCCTGTAGTTGGAATCATAGATTATCTAGCCATTTCAGATTGGCTTCTTTCATTTAGTAATA
TCATTTAGTAGCTCATTTTTAAAGTGCAAAATAACATTCCATTGTCTGGATATATTACAGCTTATCCATT
CACCTACCGAAGGTCATCTTGGTTGCTTTCAAGTTTTGGAATTATGAGTAAAACATCTGTGTTTACTAAA
ACACAGATGCTAAAAACATCTGTGTGCAGGTGTTTGTGGGGACACAAGTTTTCAACTCTTTTGGGTAAAT
ATCAAGGAGTATGATAGGTGGAGAATATGGTAAGGGTATGCTTAGTTTTATAAGAAACTGCCAAAATGTC
TTCCAAAGTGGCTGCACCATTTTTCATCCCCATCAGCAGCGGATGAGAGTTCCTGTTGTTATACATCCTT
ATCAGCATTTGGTGTTAGCAGGGTTCTGGATTTGGGCCATTTTAATAGGTGTATAGTGGTATCTCGTTGT
TTTAATTTGCATTTCCCTGAGGACATATAATGTGCAGCATCTTTTTGTATGCTATTTTTCATTTGTATAT
CTTCTTTGACGAGGTGTCCATTACAGTCTTTGGTGCATTTTTTAATCAAGTAGTTTTCTTCTAGTTAAGT
TTTAACAGTTCTTTATATGAATATGTCTTTTTAAAATATTTTCTCCTACTCTGTGGCTTTTCTTTTAATT
CTCTTGATAGTATCCTTTGCAATGGCATAGTAGGAGATAATATGGGTGACTTTTCTGTTAAAAGGTTTAT
GGAGGCCGGGCGCAGTGGCTCATGCCTGTAATCCCAGCACTTTGGGAGACCGAGGTAGGCGGATCACCTG
AGGTCAGGAGTTCAAGACCAGCCTGACCAACATGGAGAAACCCCATCTCTACTAAAAATACAAAATTAGC
CAGGTGTGGTGGTGCATGCCTGTGATCCCAGCTACTCAGGAGACTGAGGCAGGAGAATCGCTTGAACTCG
GGAGGCAGTGGTTGCCGTGAGCCGAGATTGCACCATTGCACTCCAGCCTGGGCAACTAGAGAAAAACTCC
ATCACAAAAAAAAAAAAAAAAAAAAAAAAAAAAAAAAAAAAAGGCTTCTGGATTAGCAGAAGGCATTTAC
AGAGTTTAATACTTTCCTGTCCCTTACCTCTTACCTGCAAAGTTTATTAGAAGATGGATTTTTAACATCT
AGGCTGAAATACTACAGTTAGAGCCATTTGTGTCACACTCCTGAATGTAGTCTAGTCTGTATTATTAAAA
TTTTAAAATAAACACCGAAAAGACAGACACACACGTACACACACACTCACCCTTCATTCCTCTTCCTTAG
GATGAAGTAAAATTTGTTAAGTTATATTAAGCCTCTAAACAAACTGCTTTATGGCTCCAGCAGCTTTGTG
TTGTTTCTTATCTTTGTAACATTTTGCCACAGGTACAGTATATAATGAAATAGGTGGGGGTGGCTAGTGG
AGAGGTTGTAACACTGTCAATGAGAAGGCACCCGGGATTTGTCCGTCTGACCTGCTCAGTCAGAGTAGTG
GAGCCACCCGGAGTTCTAGGGAGCATCAGTCTAGGAGGGAGATGGGCAATGTTGCATCTCCTGGAGGCCA
GCACAGTTCAAAGTTGGTTTTCTGAGTCTCGGTCTTGTGGTTTTAGGATTTTATGGCTTTTGGTGGGTAT
GTTTGGGAAGGAAGAGCCTTCATTCCTTGCTTAACCTGTGCAAATAATCTTCAAGGACTTTGATCACTGG
GCAGAGGGATAGGAGACTGCCATCTGTCCTCCAGATGAAAGGTTTCTTCTTGGCAGGAGCACTACAGAGA
TGGAAGTTGTGATTTTTTGGTTGTTTTGACTTTATGCTTCTAGAAAGGATCTTTTAAAAAATTCTTATAG
CTCTTTTGTTTTCTTTTCTTCTTTATGGTTTCTTTCCTTTTTAGTGATAGAAGCTGTGGCCAGCTCTTCA
TGTGAGTTCACCCACTTGCCCCTCCCAAGGCAGGCACCCTGATATAGACCAGTTTAGGTGGAGGTTTAGG
GTCTTCCTTGCTCACCTTTGAGCCAGGCTGGGCTGCCAATTTCTGGCTGAGCTGACTTGACCTTTAAAAA
AAACAAACTTGATATTATCTTGGCTCCTACAGAGAAGACATGCAGTGTGGGGCTGGGGGCTGGGGGTGGA
GCAGGGTTTGGTTGTACTCCCATTCATTAGTTCACCTCTTTTTCTGAGTTCCTATGCTGGGTATCCTGAC
TGACCCTTATACACTGTACCTGGGATAATGCTCCTGGAAAAGCATGTCTTTGCAACCCCATTACTGTCAT
CATGAACAAAATTAGAATGGAAAGCAAATTTTTGTTACAAAAAAAAGTTTAGGGGTAAACAGACCTGCAG
ATTTTTACAACTGCTATGTTTCTTGGTATTCTTCTGTGTGTTTAATCACTGGACAGATGTAAGGGTGAAT
TTTATTGTTCCCTTGGTGCATGTTTAACCTGTATCTTAGCTATCTGTTAAGGGGTTTAACTTGGGCTGTA
TATCTTCTTTAAGAGCTTGAAAGGGAATTTAGAGTGGCATATCTATTTTCATTGCAGCGTTAGATGTACG
GTAAGTTAAGCCATTTTTGTGAAAATTGAAAGCGTAATGTTAAAAGTGTCCAGTTTTTTAGCCATTTGTG
CACATTAAAAATAACTTGCTATTTATGTTCAGTGGTTTCCTTGTTAGACGATGTAGGGGACTTGCCACAA
CACATGCAACAGATTATAAAAAGACTGCAAATTTAATAATTACAACATGGCACAGACCGCGCTGGCAGAT
ACCGAGATCCAGCACACTCTTTAATCATCATGCTTCCCATAGTGTGGGTGGTCATGGTCCGCAGACATTT
CAGGCAGGCCAGTCCCGTTGCAAGAGCACAAGTAAACAGCATTCCACAGGCTTTTCAGGATTTTTAATTT
GAAAAAGTATAGCATTTCTTTTAGGTTGCTTTTCCTTTCTCCTTCCTCCTTCTCCCGCCTCCTCCTCTAA
ATTTTCGTTGTTTAGAATGTTTGCTTTTTCAGCAAAACCTTTTTACATATGATGCTTTTGTGTATTTCTT
TTTCCAGCTTTTATTTGGGGCAACAAAATAAAACATTTCTTAATACTCCTGAAGCCTGTTTCAGCACAAA
AGCTGAAAATGACCGGTAGGATATTTTGCTCTTTGGAGAAGTTAGCAACTTTTCATTATAAGTCCTGGTA
AATTGAATTTAATCAATTTATTTCCTTACTATTTTTTTAAAATTTAACTTTTACCGCTTGTTTCCTTTGA
TGTTTGTTTGATTAAATGAAAATTTGAGTCATCTGCAAAGTTTTTCTTCCTCATTCAAAAATGTTGTTCT
TGCTAGCCTGTTTTGTTCTTCTACTTTCCATTTTTTTGAAGCTTTTATGATCTTAGTCTTTAATGTTAAT
GAATATTGATAAAAGAAGCAAGTCACTAGTTTTTGTTGATTAATGAGAATTATGGAGTTTAGTAAAGCTC
TTGGTTAGTTTCTCTCGGGAGGAGTTATTTTAAGTAATAAATGTGTACAAATGGAAATGTATCATTCCGA
AGCTTTAACATTTTCTCTGAAAGTGTTTTTTTTTTTTAAAGAAAAACTTGGAAAAACACAGATATTTTAA
TAAAGAACAGTTAATATTACAGATGCGCACCTTTCTCTTCTTTCCTTCCCTAGGGATTTCCTTAAGGGAT
GCTGTCTTGTTGCTTTAAATTATAGACACTGTTCCTAGTCTTCATATATATAATTAAAGACAGTTGTCAA
GTTTTTTAAAATATAAAATATGACATCTTAGCTGCTATTTTATATAGGCACACATAATTGAGTAAAAGAA
CTATGTAAAAAAATACTTTAAATGTATATATCATAAGATACAGTCCTTACTAAAGTCATAGTATACTATA
CCTTTTTTTACATAAGCTGATTAAAAATGAATTGCTAACAATTAGAATTTATAATGACATTGTCAAAACC
AGCTTAAAGAGAAAGTGGTGTGATTATACATTTTAGAAGGAATAACTTCCTTTTACAATCATACCTGTAT
AGAATGCAGTGCTATACATAATTTTTGGAGTTTTGTTATTGTTCATGAAAGCATTTTAAAAATGCTTTAG
TCATTTTCATTTGTGATAAAGCTAATCTAATTCTCTATTCTGATGCTTCTTGCCAAGCTTTAGATCATAA
CAGCTTAATTTAAGACAAAAATTGCTGGCTAATCCCCTTTGGTATTTTGAAGCTATTTTGGGCTCCCTTT
GTTCTATGGTACGTTCCATTCTGTTTATGGGGAGCCATCGGGTGTTTGATGTGGGACCCTGTGGCAGAGG
AGATTGGAACTCCAGAGTCTGGGTGCAACTGGGACAAGTTAAGGATGCTGAATTTAACTTGCTCTTGGAT
TTGTGTTTCTTCTATATAAATTGAAGGATTACATTTAATTTTAGATGGAGTAGGGGTAAGGCAGATTGGG
TTTGATTTTCGAAGTCAACATTTGGAGTTTGGTAACCATATCAAAAGCGAGGATTTTGACAGATTTAATT
TTCATATTACTGGTTAACATATACTTATGACTAAGTGTTTAGTCTCCCCTTTCCTGAGCTTCCTGATGGG
TGTCAGATTTGCATTAAGACACCAAGGCCAACCCGCCTACACACAAAAATTGTGTTTTGAAAGTTCTTAA
TCCCTCATTTCAGACTCATGTTGTGGTTCCCACCAAGTGGGCTGCCTGAGACCCAGTGGAAGTTTAGCAG
ATTTCTATTCACAGGCATGCTCTCAGAACATCTCCCAGTTACCAATGCCTAATTAATGTCCTTGTGATGG
ATATCCATGGGTCTATATAGATGAAGCTTCTTGAACTCCATTCATAAAACAGATATATAGCCATATAATA
GACATGAGTAATGGACGCCTAGAAGACTAAATGTTCTATGTATTGTTTTTTGTGGTTCCTTCAGTGAAAT
TAAAGGATCAATGTTTTACGTAAGTTTAAACTACTATTGTTGAAATTATTTTACTTTCCTGGCCTTTTGT
CTATAAAGAACACATTAGCATATACTAACCGTGTCTTATTATTGGACACAGTGCTTCTTTGCTCCTGGGG
ATATTTACAGTTATCCCCTCCACTCTGTCCTAAGTGTAAGTATACATTGTTGTGTTTTTGTGTTATAGAT
TAACAGCTCTGAAAATGTGGTCCAAGGACTCTGGGGTCTTCAAGACTATTTCAGGAGGCCTACAAGGTCC
TCCCTTTTCCAGCTATGTAATTTTATTATGCTGGATTTTCTTTATAAATTGAACTAAAGCAACATATCAC
AACAGATTGAATGCCGCAACAGATAGGAGAACACAGCTGTCTTCTATTGAGACAAACATTAAAGAGATTT
GCAAAAAAATGTAAAACAGTGCCACTCAAAATTTTTTGTTTTGTTTTGGAAAACATATTTTTTCATAAAT
AATATTTTTGTTAACATGTAAAGGGTTATTATCTCATATTAATTACTAGATAAATATTTTAAATTTTTCT
GAGGCTTGATTACAAGTACAGTAAATAGTGATAGGTAAAACCAAAATAAAAAGTGCTTTGGGATCCCAAG
ACCAGAAAACTTAAGAACTGCTAATATATACAATTGGGATTATTGCTGTGGTTCTAAAATCAAACTTCCA
TTGGCTTCTCTGAGAGTTTTAGCCAAGTGAGCATATAAAATGTAGTGAAATTAGCCTGCGGTTACGAGGT
TTTAATGAACTGTCTCCTTCACATTTTCAGGTGTTTTTTTTTTTTTTTTTTGAGACAGGGTCTTGTTCTG
TCACCCAGCCACGAGGCTGAAGTGCCCTCCAGCTCTGGGCTCAAGCGATCCTTCCACCTCAGCCTTCCAA
GTAGCTGGAACCACAGGCACATGCCACCATGCCCAGCTATATTTTTGTATTTTTTTGTAAAGATGGGGTC
TTGCTATGTTGCCCAGGCTGGTCTTGAACTCCTGAGCTCAAGTGATCTGCCCACTTCAGCCTCCCAAAGT
TCTGGGATTACAAAGTGGCATGAGCCACTGTGCCTGGCCTCATGTGTCTTTTTATAAATAAATTATCAAG
CAAAGCTGCCACTGTCCTCTTGTAAATTTAATAAGGAGTCATAGGTTTTAGAGTTATGATCCCTTTTACT
TATTTAGCTGATATTTCTTTGCAGTTTGACATAATCTGGACCCATCTGAATTGTAATATGTGACTTTTAA
CAAACATTGTAGATTACCATCTTTCATATGTGATCTCATGCCATTCATGGAATCTATGGCAATTTCAGTA
AGGATAGAACTCATCATTCTTGTGTATACTTGGGATGTTTCTAACCACATTTGCCTCAGTTACCTAATAT
TTTTTGCTTTTCAATTGATTTCTAGTTTGGGGATGAAGAAGAAAAAGAATGTAACTGGCAAAATAGCCAT
TATTTTTTGCCTTTATTCTTATTTCACACTTTCTATTTTTAGTTTATCTTGCTTTGTAACCAATCCTTTA
GAATGTAATTACTTCTTAATTGGAAGGGGCTAACTGCCCCACAGAAGAAACTTCTGTAATTAGAAAAAGA
GTCCCATAACATCTGATCTGGTTCTTTCGCCCTGAGCATATTTGTTGGCAATTTATGGCAAGGTTGACTC
ACTGGTGGGAAGCTGCCTCACTCCTAACCTGTGCTAGAAAAGCTGCCTCGTTCTAGAGGCAGCCCTGCCC
CTGTTAAGCCCAGTTCTGCTGCCTCACTCCTAACCTGTGCTAGAAAAGCTGGCTCTTTCTAGAGGCAGTC
CTGCCCCCTGTTAAGCCCAGTTCTGCTGCCTCACTCCTAACCTGTGCTAGAAAAGCTGGCTCTTTCTAGA
GGCAGCCCTGCCCCGTTAAGCCCAGTTCCCTCTGCCCTGGCATCTCCACCTCTACTGGGGCAGTTTCCCC
AGTTCTCTTCCCAGCCAGCAGCACCCCTGGAGCCTTCCTTTTAGTCTCCGGTCACATGAGTGAAGAGGAA
ATTTTCCTCCTTCTCCGAGCTTAATACAGCAGTTTTATATATTTAACATTGTAGGTGATTTGCATGGCAA
TGAGCTTATTAGGCTGTTTTTTGAGTGCTGCCAAACACCTAATTGATTTTTTCAGTTTTCTTCCAAGATT
ATAGTTGTCAAGAACCATCTGCCTTATATATTACATTAAATGGGTTAAAGCTGCAAATGCTTCTTTAAGC
AAAATATAAGTGTGTATCTTATAGAAAGTAATGCAATATCACCAGTCTGAGGTTTGGAGTGTATTAAGGT
ACTTGGTTTCTGGAAATCTTTTTAAGCTTGGAAACCTTTAAACCTGCGACTTAAAAAAGGATCTACAGCT
ATCTAAGTAGGTGGCCTTCTGTGTCTTGGCTAGTTTTAGAGTGTGGTGTTTGTTTCTTACGCAAAAAGGA
ACAAATTTAAATCACTAGGTACTCAACCAACAGATTTCAGTGTGGATTAGTTAAGAAATAATTGTAGTTC
TTGTGTGGTGGCACAAATTTTTTTCCCTACATTTTGTTTATGCAGACTGTCTTTTTTTCCTTCATCCTTT
TCTTTTTTTCACTGAAAAAGGAAATCGCAATTTTGTTAGTGGATTGCTTTGAAGAAAATACGGAGGGAAA
GAGAGAACCAGACTTGACTTGTGATTACTTTTCATGAGTGTGGATAAGTGTGGGCTTCAAGACTTGCCAG
GCCTTCAATTCTTTGGCTTGCTTTTTTTCTTAACATTTCCAAAGGTTTTGTTAGTGACTTTCAAATGGAA
CTTCACGCCTTGGAACAGTTCCATTAAAAAGTTGTGTGGTGTTAGCCACAGTAGCAGCATGGTTTATTTT
TCCTGTACTAGTTTCACAGAGGTTAACTGAGCTGGAAATGATGAGAGACTAGCAGTGCAAGCGGGTGGGT
GTGTGCTATGTGTATGCTTTTTCCCTCTTGATTTGTGCAAAAAAAAAAATTTTTTTTATTGGCATTAACG
TGTACTTCAAATGTTGTTTAAAAAGGAAGCAAGTCAAATTATCCTAAAATGCTTTATACTACATTGTTGA
AAACTTTCAAAAATGTTCTCTTTTGTTAATTGTATTTTTACATTGCATAGTACCAGCCCACATTTATAAA
AGGCAATTCATTATAGAGATTTGCTTTCTTGATGGGAAATGTACTACAGTTTACAGCTCAGCATAATCTT
AGTTGACGTGTTCTGGAGAGTGCTTTTTATCTGTCTGAATTTAAAAGAACACTTGACTATGTTCATGATT
GAGACGGTTTTATTCCTGGTTCAAATAAATATCTTGAAGTTAAAATATGCGCCTTTTATTCCTTAGGTGA
GTATATTTTTTAAGAAAACATGGTCTGTTATTTATATGTCATCCGCTTGTTATATGCATAAGTTACTTGA
AGAATAGTGAATTTCTAATGAAAATAACAATTACATTAATACTTTAGATTTTAATGCTGTTTTGTAAATT
TTGAAGCAAATTCATATATCTCATTTAATTCTCACAACAACCTCATGATACCCCAGGGCAGATATAATTA
TCTCCATGTTATAGATGAGATGAGAAATGTAAGGCATTTGCCTGAAATTCCCCATTTGGTAAATGAAGGA
GCTGGATCAAGTCCTCACATCCCTGACTCTGCTGTTCGTCTTGCCACTCTCCCGTAAACAGCTGCTTAGA
CCTCCTTGATAGTTTACACCATGGCTTGTGGATGGACAACTGCCATAAAATTGTTTATAACACACACTTA
TTGACTGCTTACAAAGTTTCGCACATTTTGAAATGAGACTTACCCCAATTAATTCCCTCACCAACTCTCT
GAGTTAGGAACTATTATTATATCCCTATTTTGTGGTTGAGAAAACTCAGGTTCATTAGATAGGTGAGTTG
AGCTTCCTAGGGTAATGTAGCCAGTAAGTGGTGGGTACTAATGTGAACCCCAGCAGCTTTGCTCTGAGAT
CCAGGGTCCCTGACTGCTACCGTGCATGAACTCCCAGTGGTTCATGTGCCGGATTTTCAAACGCCAGGTA
TTCACTGGTATTTGTAATAGCATCTGTTTGCATTCATCTGTAATCTTGAAGATCTGACCATGAAAACTTA
GAAATGAGAGGTTTATCCTCTGAATTTACACCGAAATAAAAGAAAAATTTTAAATATAAAATACATATTA
TTATCAGTAGCTATACTGCATGGAGCAGATAGCTACTAACTTGTATAAATCATCCTAAAAATAAGATAAG
TGTTTATTAAGAAACAAAGAAGGTTCATATAAGCTGGGGAAATTTTTTTTATCTAATAAATGTCCTCTCT
GTTGTATAGAGGGTTCTTAAAATGTAAAACTTTTGGGGAGAAATGAAGTATGAAAGGTTTCACCTAAACA
GCAGAGCCATTGTACCAAGGCGAATGTATATACCTTTCAGAACTCTGGAAGTCACTACTGTAAACACTCA
TCCTTGGACTGGAAACCAGGAGCCTCTGGTGGTAGTTTTTTCTCTGTTCCCCCACCCCTCCATTCAACAA
ATGGAATGCCTGTGTGCGCTGGGCACTGTCTGTGCTGTGCTGGGCACACAGTGGTGCTCAGATGGTTCAG
GAGGCAAACAGCCATCAGTCAGTACTTCCTTAGACCGAGAGACCACGCCCTGGGAGAAGGGTGGCTAGTT
AGGTCATGGCAAACGGACATGAGGTGGTCTTGCTGGAGCTGACACCGGAAGAGCAATTCGGGCTTTAGTA
GGTGAGGAGAGGGAAGGGGATGTAGGAGGTGAGCAGAACGCACTGGGAGGAGGCATGGTGAGTAGGAGGG
GCCCAAGGAGGCAGAGAGGGCTGTGGGTGCAGGGTGGGGCTGCAGGGTGGGGCTGCAGAGTCAATCCTGG
GAAGGCTTCTCTATATCTAAGCAGCAGCAGGAGGAAAGGATTTTAAGTACTAGGGGTGAGGTGGGGGGCT
TCGAAAACTGGCCAGAGTGCAGTCTGTGGAACAGATTGTTTTAAGGATTTGATACATGGCAAGGTCTAAA
CCCATTCAGATCCAACACCGGTTTTTAATAACAGATGTTTTATAATACCTGGTTGGAGATCCTGAAATGA
AATTCAAAATACTCTTTCAAAAAAAATTCATATATTGCCTTTACTACAATATAAAGAAAACTAGGTCAGG
CACAGTAGCTCACACCTGTAATCTCAGTACTGCAAGAGGATCACTTGAGCCCAGGAGTTCCTGGCCAGCC
TGGGCAACATAGCGACACCCTGGCTCTACAAAAAAAGTTTAAAAGTTAGCCAGGTATGCTGGCGCACACC
TGTTGTCCTAGCTACTTGGGAGGCTGAGGCGGGAGGATCACCTGAGCCCAGGAGGTGGAGGCTGTAATAA
GCTGTAATGGCGCCACTGCACTCCAGTCTGGACCTGTTTCTAGTAAGACTCTGTCTCCCCTCCCCCACAC
ACACAAAAAAAATTTTAATAAGATAATGTACATTTCCAGTGTAAATACTTGGGTCTTATGAGACTGGGAG
ACATCATGAACGGATGCATGCATCTACACATGGGCTCTCTGAGAATGTGACCACTGCAAATTCAGGCTGT
TAGAGGAGGGCCTCATGTGGCCTTGCCTTCCAGGAGGTGATTTTCCAAAACGATAGACAACTCTTGATAA
TGCTATGATGAAACTGCTGTAAAATCTTCCCTGACCAACAGGGTAATTACATTTTCAGAGAAGTCAGAGG
ATGCCAGAACATGCAAAACATATTTTGTATTTCTATGTAAAACAGCTTTAGGCTCTAAAAGAAGGTTCCA
CATACATGATGATACAGTGGAACGTCTGAAAATTGTGTGTGACAGTTTGCTATTATATGGTATATTTCTA
GTTTTCCTTAACCACTAAATGCCAGTAAAGCCACAAAAATGTTTTAATAGCAGATTAGTGGATTTTGAAT
TGGCCCCATCAAGAGTCACTGTTGTGTTCTATAGCCCTTTCCCACCTAAGTCCCAAGCTAAAGACAGTGA
ATTGTGGTAGCATTTCAGAAATTCCATCTCCACTAGCTGCTGGGTCATGACGTGTGTCTCACTCCTGCGA
GTGCTGGCTTAGGGGCCTTTAACACCTCTGAGCCAAGACTCTGGCATGGAGCCCTTTGCCCATCCTAGGA
GTCTTGAGTCTCAACCCGTGATTATATTCTTGATTGTAGACCTTCTCCCCTGATGAGATCAACCCTTTCT
CTAGGGACTCCTGGAATTTGGCCACTGATGCCTACTTGACTACTATTTTTGGGTCCTGTGGGGACCTCTG
CTGTGTTGTTCCACCCACTCAGAAGGGCTTTTTGGATGGCTTCATCAGTTCAGAACATGTCCAGGCTTGA
GACTTTGTTTCTTGGCCTTCTGCCTGGTAACTCCTTTATGTCAACTCAGGGCTTGGCATCTTGGCTGCAG
CAGCAGTAGAAGGTCCTCTTGGGCCCAGCACCACTGGCCTTCCTCAGGAGTCAGCTCTGCAATTTAACTA
ACAAGCCACGATTCCTGCAGACTGTGAGGGCCATGTGCAAGATAAGCATGATGACAGGGCTAGATGCACA
GTACCTGGCAGAGAAAGCTTTCAGTACGTCTCAACTATAACTACCATACAGTTCCTTTATTATATTGGGG
TTTGTTTGTTTGTGACGGGGTCTGATTCTTTCACCCAGGCTGGAGTGCAGTGGCACAATCATGGCTCATT
GTAGCCTTGACCTCCCAGACCCAAGCAGTCCTCCAACCTCAGCTTCTCAAGTAGTTGGGACTACAGATGC
GTGCCACCATACCCGGCTAATTTAAAAAGTTTTTTTGTAAAGACAGGGTCTCCCTTTGTTACCCAGGCTG
GTCTGGAACTTCTGGGCTCAAACAGTCCTCCTACCTCGGCCTCTCAAGGTGCTGGGATTACAGGTGTGAG
CCACTGCACTTGGCTATAGTTGGCTTTTTTAACTTGAGGACCTTGGGCCCTGGTGTTCATGGATGGGCTT
CAAAGGGTCAGAAAACTCCATGAAATTGTGTGGAAATATTGTAGGCATATATAGTTTTCCTGGAAGAGGG
TTCACAACTTTCATCAGGTACTCAGGATAGGTCATGAGGGACTTCTGTTTTTAGCAATTGCAAACTCAGA
AATCAAGTGACACCTCCTCCTAAGGATGAGAAAACTCAGACTAAATATGAAAAATATGCTTCAAAGCTTG
AAAAAGCTCATGATATAGTAAATATGTTCTCAGTCAGAGTCCAGGGAAAGGTAGAGGCCACAGGAGGTGA
GCCTATGGTTTAGGACTAGGGGTGTCTGCCAGTTACAAAGGAGACGATTAAGCAGCAAGGTGCTTTTGGA
GCATCACAGGACTAGGAGGGCAGAAATTAGAGTCCGTGGTCTGCCAACTGAGCACATCCTGGTGAACCTT
TTACTTGAGGTTGGGATCTTGAAGGGAAGCACCTGAAGAGTAAATGTGAACCGAAGCAGACAGGCCCTCA
GAGAGTGTGCAGTTTACCCTCAGATCATCTCAGTCACTGACCCTGGATTAGGGTAATCCTGGATTGCTAG
TGATTCAGGTGTGACAGAAGCCTCTTTGGAGGACGACAATATTATCTTGGTATCTCAAATTATTTCTGCC
AGCAGTTTTGCAAGTGTAGTGTCGTGTCTGGTGTAGATAAAAATAACTAGGTATACAAAGAGCCAGGATA
ACAAAATTGAATCAGTAGAAATAGCAGACAATAAAACAGACACATAGGGGCTCCAGATATTAGCGTTAAG
ACTCAGACTTTAAAATATTATAAATCAATCAGTGTAATCCACCATATTAAAGAAGAAAAAGCATGTGATT
ATATCAATTGATGCAGGAAAAATGCATTTAATAAGATCCAATACCTATTCATGATTTAAAAAAAAAATCT
CCCAATAAATTAATAGTGGGGAATTACCTCAACTTCATAAAGGGCACCTCCCAAACACGTAGAGTGAATA
TCATATTTAATAGTGAAAGATTGAATACTTCCCCCCAAGACCAGAAACAAGGCAAGGAGATTCGTTCTTA
TAACACTGGAAGTTCAAGACACTGTTATCAGTCAAGAAAAAGAAATAAAAGGCCTGCATATTGTAAAGGA
AGAAAAACCATTGCTATTTGCAAATGATATAATCGTTTACATAGAATATCTTAAAGAATCTACCAAAAAC
CAAAATAAATCAATTGTTAGAATTGATAAATCAGGTCAGCAGAGCTGCAGGATACAAGATCAACACACAA
CAGTCAACTCCATCTCCGTATACTATCAATGAACATTTGAAAACCAAAACTAAAAACACAATATATTTTA
GAGTCACTCCAAAGAAAATAAAATACTTAGGTATATACATAACAAAATGGGATCTGTTTAATGAAAGTTA
CAAAGTGCTGATGAAATAAATGCTTAAAGACTGAAATAAATGGAGAGATATTTAGTGATGATGCATTGAA
GACTCAACTTAGTAAAGATGTCAGTTCTTCTCAAATTGATCTATAACTTTAAAGCATTTCCTATCAAAAT
TCTAGCAAGGCTTTTTATATTCATAGGCAGACTTATTCTAGAATTTATAGGGAAAAGCACAAGCCCTAGA
ATAGCTATAACAATTTTGACAAGAAAACGGGGAGAAATCACTGTATTTAATACTAAGTCTTTACTGTGAT
AGCTGCAATAATTAGATCCTGTGATATTGACAGAGGCGTGGACACATAGATGAATGGAACAGAACAGAGA
ACCTGGAAATAGACCCACACAAATAAGTTCAACTGGTTTTTGACAAAGGTACAGAAGCAATTCAATGGAG
AAATTTCAACAAATGGTGCTTTTGCAGTTGGACATCAACCTAAACCTCACAACTTATACAAAAATTAACT
CAAATGGATCAGGGATTTAAATGTAAAATGTAAAACTATAAAACTTTTCAAAAAAAAAAAAAAAGGCTCT
GGAACTAGGCATAGTTGGTTTTTTTTTTTTTTTTTTTTTTTTTGAGACGGAGTCTCACTCTGTTGCCAGG
CTGGAGTGCAGTGGCGCCATCTTGGCTCACTGCAACCTCTGCCTCCCGGGTTCAAGTGATTCTCTTGCCT
CAGCCTCCCAAGTAGCTGGGACTGCAGGCACGTGCCACCACGCCAAGCTAATTTTTGTATTTTTAGTAGA
GACGGGGTTTCACCATGTTGGCCAGGCTGGTCTCCATCTCCTGACCTCGTGATCTGTCTGCCTCAGCCTC
CCAAAGTGCTGGGATTACAGGCGTGAGCCACCAAGCCTGGCCAAACTAGGCATAGTTTCTATACTTGCTA
CCCCAAGCATAATATGTAAAAAGAAAAATCAACTGTGTTAGGACCTCATCAAAACTAAAACCTTGTGTTC
TGTGAAAGCCCACGTGAAGGACAATGGCAGACTGGGAGGAGATATTTGCAAGCCACAAATAACAAAGGAC
TAGTATCTAGATAATCAAGAAATCTCAAAACTCAATGGTAAAAAACAACCCAGTTAGAAAATGGGCAAAC
ACTATTTCACGAAAAGGTGAAATAAGTTCATGAGCAAAAAAAAAATGGCAAATAAACACATGACAAGATA
TTTCACATCATTAGCCATTGGGGAAGTGAAAAAATAAAACCACAGTGGGCTATCACTACGCACCTATCAA
ATGGCTAAAATAAAAGCCAGCGAGAACTTAAAATGCTGGTGAAGATGTGGAGGAACTGCGTTACTCATGC
ATTTTTGGTAGGAACATGGCTGTGGATAGTTTTGTACCCAAGGCAAATGCTCTCCTGCATCTTCTTAGTG
CCAGCCTTGACAGGCTGCTTGTTGACTTCTTACAGAAGTAATGGAAGCCAGGAGACAGTGTAATGATATC
TTCAAATTCATGGGGGTGGAGGAATCCATTAAAAATGAGACATTTTAGGTAAACGAAAAGTAGGAGAATT
TGTCACCAGCAGAGCTCCACAAAGGGAGTACTAAGACTGTTCTTCAAGCAGAAGGAAAATGACCAAAGTA
ATCCTCCATCTCCAAAAATATACGAAATTTAATTAATCTAGTTTCCATGCTCTGCCTGGCCCAGTGAGTT
TTGGCCCTGGCTGCACATCAGAATTACCAATGGAATTTAAAAAAAAACACAACCCTCTATATCCTTCTTC
AGTTGGTTTGGTGTATGGCTCAGGGTCACTAATGGTACTGGTGTGCAGCCTTGGAAGTAACAGGGTGGAC
TCAGAAGGAGAGAAGAAAGTGGTGTCATGGCTTAAATGGAACACACTGTCCTCCTGGTTCCTGGGCCTCA
CCTTTATGCCTGGTATGTCTTCCTGTGGCAGCATTCGTTCTGGCTAGGCTGAAATGACCCTCCGTAGCTC
TGAGTTCTCCCTCTTTTGCTCCAGTGAGCACCAAATCCCGTCTATCACACCTGTGTGGTATTTCACACTT
TTATCCTTTTCTCTTCATTGCCATTGCCGCCCTTGCAATTTGGGCACCCTTTACATTTTGACTGTGATAG
CTGGTCAGTGGCAAAAACCGCAGTTACTTTTGCACCAACCTGTAGTATCTCAGTTTCCAATGTCATCTTC
CTCTTTCAGTTTGTCTTGCACAGTCTTACCAACCCAATTTTTGTAAAGCATAACTCATGCTACTGCCCCA
TTTAGAGACATTTTTAGTGCTCCCCTCCCCAATGTTCACAAAATAATCTCAAATTTAATACTTTTGAAAA
CAAAATCAAGTTGTTTACCTATTGAATACCCTTTGATCACACCCACGGCTCCTGTTACCACAGCATACAG
GATGCGACATGATTGGAGGTCTGTTTCGTCTCCACCCTCATCTCATAGCACTCCTTCTTCTGCTCGCCAT
GGCCTAGCTAGAGTCTGGGTTTTTCCTTGCCTTTGGGCTGAACTTGCCGATTACTCCACAAGCTGTTTGC
ATGCTCTTTTCTTACCTTTCAGGTCTTGATCCCATGCTTAGTCTTTGGAGTAGCGCCCCCTTGCTTATCT
AAAGTGAATCGTATCCATTTCTAATCTAATCAGCATGCTCTTCATTTCTTTTATTGTTGGTCATCTTCAT
CTTGTTGACTTCTATATGTGTTCGTTGCCTCTTCCCTTTTCCCTCACTGGAATGTAAACCCCTTGAAGGC
AGCCACCTCAACTCTTGTTGGCTGCTACATCTCCAAAGCCAGGTACATAGGAGGTGCTCAATAAATATCC
CCTAATCTATTTTGAATATAGATACACTATAGTAAAGATTTTATAACAAAGGAACTTTCTGACAAGAGAA
TCTTGTTTGTTCAGTGATTTGGACAATGTAGTACTATGTAGTTGGTTATTGAAATTCTGCCTGTTCCCAT
TCTTACATGTAACCTTTGGATGAATGAAGGGCAGGAAATGTGGTAGGAAATGAAACCGTATAGGAAAAAG
CTGATGTCTAGGTTGGACAAAGAAGCTGGAAATAGAATTCTGTCATTTCTGCAAGATCCAAGAGTCTCTA
CTTTGACTATACCTTTATGTCTCTGAAATTTCTGTAATCTTTTTCTGTTGCTTCAATAATAGTAATTTTA
AAAATTTGATAGTCAAATTCCAGGTACTGTTTTTAAAGGGACATTTCTAGAGACGTTATATTTAATAACA
TATTTGAATGGTGTTCTATAGTTTGTTCATTCGTAATTGCGTTGAACAGCTCCCTGTGAAAAATGTTATA
TCCTCACTTTGAAAATGAGAAAAGTGAGGCCCTAAATGTTAAGAAACATTTAAAAAGCTAGTTAAGTTGG
GAAACTGGGACTGAGGTCTTTGAATGTCTAATCCAGTTTCTGTGTCACTACTGGGTTTCCCATCCCTGGG
GCGTCAAGTTTCAACTGAAAGTACCTGTGGCAGGAACTGGAGATGTAACAGACTTGCTTGTCTCTCAAAT
CTGGGAACAAGTGAAGGTGACTACCACCAGGTCACCTTCACATAGCAACACGATGTCTACCTGGTGATGT
CCTTGGTCGTTGTCCCTGCTCCTCCTGTCGTTGGTCCCAGCAGATGTAGTTGTCCAGCTTGACATGGCTG
AAGCACCGTGGGAATCGATTTTAGATATCACATCCAAGGTGTTCCTCCCATTGTTCCCTGCATCTTCATT
CTTAAAAAGGAAATAAGTCTCATCAAAAGTATTACTGCATTCAGAACAACTCACATTTCTTACTCATAAA
AATTGTTTTGGCCTTAAGAGAGATATGTCCTAACATTCCCTCCTCTTGCTGCCTTATGGAAAATGGATCT
CGGGGGAGGAAGCAAGAATAAAATGAGAAAAACGAGACTGGAGGCTGTGACAGCATGCAGGTGAGAGATC
ATGGTGGCTGGGATCACGGTGAAAGCAGTGCCCTGGAGAGAAGCAGACCTATCCAGAGTGTAGTTGAGCT
GTAATTGGCAGGACTTGCAGATGGGCTGCGTATGGAGGATGAGGGAGAGGATTTGAAGGATGAGTCGAAG
GTAACATCTAAAATGTTGACTGGAGCAACCATGTGGTTGGTAGAATCATGTTCTTGGGTGGCAAAGATGA
AAAAGGGGCTTGGGGATGGCTGGGCTAAGAATCTTAGTTGTGTTAAATTTGAGATACCAATTGCACAGTT
GCAGATGTCAGGCAAGCTAGTTGCATATATGATCTGGAGCATAGGAGAGATGGCAGGGCTGGAACTAGAA
TTTTGGAAGGTGTGGTCACCCTATAGAAGTAGGATGCTGGCCAGGTGTGGTAGCCCATGCCTGTAATTCC
AGCACTTTGGGAGGCAAGAGGATTGCTTGAGCTGAGGAAGTCTTGGCTGCAGTGAACCAAGATCACACCA
CTGCACTCCATCCTGGGAAACAGTGAGACCCTGTCTCAAAAAAAAAAAAAAAAAAAAAAAAAGGAAGTAG
GATGGTGACTGGGGCTGGGCTGGGCTGGGCTGGGCTGGGCCTTGTGAGGTTTGAACCCCACCCTGGGCCA
TAGGAGGGAGCACTCAGGCTTTCTTAGTCTTCTGTTGACCTGCCCAGATGCAAGGCCGAAGGGGAAGTTG
GCCTGGAGTTCAGTCTTTTATGACAGAAGACCCCAGCATAGAAATGGCATCCACAATCATGGGACTGTAC
AAAATCTCATGGTCTTGAAAGCCACTGTCCTGACACCTCAAGACTGGAGTCCAGCCCTTCATGACAGAAG
ACCTCAGCATAGAAATGGCATCCAAAATCCCGGGACTATACAAAATCTCTTGGGGTGCAACCTTTGTGAC
AGAAGACCCCAGCACAGAAATGGCATCCAAAACCACGGGACTATACAAAATTTCCTGGAGTCCAGCCCTT
CATGACAGAAGACCTCAGCATAGAAATGGCATCTAAAATCATGGGACTGTACAAAATCTCCTGGGGATCA
CTTGAGCTCCCAGGTCTCCAGTGTCTAAAAATGGGGTTGAAGAGAAAGTACCAGCTAAGTTAGACTAGAT
GAAAAGAAAAATGTGTAGCATGCGGTGTACCAGCAGCAGAAACCAGGGCCACCCTGCTGACCTGCTCTGG
TCTCACCCACACAGTGCCAGGGCGGGCAGTGTGTGTGCATCCTGGAGATGTACAGGTGCAGCCCTGGGGG
AGAGGCAGCCCAGGGCACCGCTGAGTGGGCAAGCAAGAGGGTGCAGGGGTGGATCCCACGGTTTGTAGCA
TGGAGTTCTGACTGGTGAACTGGTAGGGATAGAAGCCAGATTAGAGAAGCCTGAAGAATGAATGTGAGTT
GCAGAAGCAAAGAAGGCAAGTTTAGACAGCTTTCTCAGGAAATTGTGCTAAATGCAGAACACAGAGATGA
GGAGTAATTAGAAGGAGGGAGAGGTAGGATCAAGGAGAACTTTTTTCTTTTTAATCTAGGTAGCTCTTCT
AGCATGTTTGTAATTGACATGAGAAATCCAGCAATGACGCGGGTCACTGCTGCGGAGGGTCCTGGCGAGG
TAGGAGTGGAGGTGATCAGGGCCCAAGTGGACAGCTGCACTTTATAGGAGCAGAGACACTCCTTCCATCT
GAACAGGAGGGAAGACAGGACATGGGGGGTGTGCGTGTGCAGGTGAGCTTGTAAATTGGTTAAATTGGTT
GGTGGTGAGATGAGTGAGTTTCTTGCTAATTGCTATTTTCTCAACTACATATGAGATGAGCTCCTCACCA
GGACTTTTGGGGAAGGGGTGGTGGTGTGATGAGAGAGGAAGCAGGAAATAATTACTCAGGAAGTGGCCAA
GCAAAATGACCAAGGATGCATAAGAAGATTGTGAGGCACTGCTGAGCCCCTTCCCTTCAAGGGTTGTGGT
CAGAAATCCAAGCCAACGCTGTCAGCTCAGTTGTCTGATATTCTCCAGCAGTGTTCAGCTGTGTGGGCAC
CAGGAAGAAAAGCTGCAGCATGGATTCACAAGGGCCAAGGTTTTACACAGCAAGTACCACTGGACTTTAG
AAAAGGGGGCAAGCCAGGGAGATGAGGGTGTTTTCAAAGGGATGATTATTTAAGCCTGGCAGAATATTTA
TCAAATGCATGATCTGCTCCTAGTGAATGTATTTAGTAAAGCACACATTTCCAGGTCTGTGTATCTACAT
ATTTCTGTATACATTCTTATAGATACGGTTTAATGCCATGGTTTCATTCAGGCTTATTTTCTAAAGCCTT
TTGGTTTTGGTTTTTGGGAAGCCTACTCTATTTCCCCCCCCGGAACTAATGAGCGAGCAAGCAAGAGGTT
CTGTAAGCAAAAGTAAAAGCTGCAGACTTTGTTATGGTTGGACCTGTGTGTGGAAATTCTGTGAATTGTG
TTCTGTCAGCATCCTGCTCACGGAATAGGCTATGTAAACAACGGGCGCTGGGAGGGTCTGTGAATGAGCA
GCCTTCCTTCCCACTCACAGACTTAACCACTGCAATTCTCCAGGTTTCTGAGTGTTTATTTCATAGTAAA
AAGGGCTCTAAAAATACCAGGGCAGTTAGGTTTTTATTGTAGATCATTTCACTTAAACTGCAATGGGACA
TATAAATTTTAAAAAGGGAAAAAAAAAACATTGAAGGCAGGATTTTGAAGCAAAAGCTACAGAAATGTGT
CTGCAGTAATTTGTTTTGGCTGTGGATGTAAAACTGCAAAAGGGATGGGAATAAATTTGTTCCATTTTTT
TAAGTTAGAGCAATTGGCGTATTTAATTGATATTCACAGTATTTGAATAGCATCCCAGAATGCACAAATA
ATATGCTATCATGAGAAAATTACATATACTGAGATGTAAATGAAAGGAAATTCTTTTTTTGAACTTAAAA
TATCATTTGTAACTTATGTGTAATTTTTTAATTAAAAATCATAGGGCACATCATACTGTAAGTTTGAGTA
TGCAGAGTGAAATGAGAAAAGGATTCAGGCAGATTAGGCTTGAGGACAGGAGGCAAAGCCCCAAGAGTCA
GTCCAGGTAGGGATTATAAATGGCAGGGTTCGGGAGGAGTGGACATAAAAATGCGAGAGAACAGGAAACT
GCAGGAAGTAACAGTTAGTGGGAAACAAAGAGAGAAGTTACTGAGAAAACAGACACAAACACTGACGTGT
TTCGGACTGCCATGTGACCACCACTGAAGGATGATAAGGTTTACTGAGTAGGAAGATTTGAAATTATGGA
CAGGAGCAATATGTACCAAGGAAAGCAAATATTTTGCTTTTCACTGGATGGAAATTTCTCCCTTCTAGGA
ACTCCTGACCTTCTTTATGGGACCAAGCAGTGTAAGTTGTAGGCTTGTGGTTCCTTCTGACTTCTTGTGT
CTTCTCCCTAGGGGCAATGATGGTGCTGGGGTGTGATGTTTTCAGTATGAAGTTGTGCTTGGAGGCAGTG
GCAAGAGCCAGTGGGAGCTTTTCATGGAGCAGGCTGCATTAACCCACGCGCTGTGTGCCTCCTGAAGCTG
TGGCAGTAGTGGCGTGACACCTGCCTTCCTGGGAGCCTGGGAAGAACCCCAGGAAGAGACAGTGAAGATT
GCATCTCTTGTGGGAGATAGAAAAGAGTTCAGTTTAGTATTGGCAGTGATAAATGCTTGCTGTGTAGGCA
AACAGAATGATGAAACAGAAGACATTAAAGACCTCTTTCTCCCCCTAATTAGGATAGGTTCTAGACATTT
GGCCTGTGTCAGAGAAACAGCCCTGGCACTGAACCATGGAAACTAACATTTATTTCGCACCTACTATGAG
CCAGATCTCTAGCCGGGCGATTTACATGAAGACACTGATGCGTAGTGACAGTAAGTGAGTTTCTTCACTG
AGTGGCAGAACTACTCATTGAACCTAAATGGGTCTGACTCCAAGTCTTGAGACGTTTCCATCCTGAAAGC
GTTCTACCTCAGGTCCTCAAGTAATCTCACCACACACGGTGGTCTAGGTCATGTCTGCACCCTGTTGTTG
GAGATTTTGAACCAGAGGAAGGGGTCACTCCGGCAGAGGTGGGGAGGTCACAGGATGACCTGGGCTTCCC
CCTATGCTGTCCTCTCCTGGTTCTTTCCCTAGTGCACAGGAGATAAAGACACACAGCATTTGAGCTATAG
TCAGGTCTGTGGCTGGTGAGCCCCAGACTTTACAGTGCAGTGGAAAGGGGGTGTTTGTCCTGTTGGAAGA
ACAGATTCTAGGGCCCACCCCCAGAGGTTTGGATTCAGTGGGCTGGGTGGGACCAAGGTGGTCTGCAGGA
TAATTCAGATTGAGGTCTCTGTACATGACATCCTGAGAAACGCTGACTTAGGCCCTCCGGTTTTCAGTCT
TGGCTCCTCCTGGGCCTCTGCATACATTTGTGGCAGACCTAGAAATGATATCGTCAGGTGAGTGTATGTA
TGCAGGACAGGCATTGGAGATGAGCCAGAGGGCAGGAGGTGGGCCTTGGCCGTGACCATGAAGAGGGAGA
AGGGAGAAGGTTAGAGGTATCCAGAACCCAGGAACTGGGGTTACGCAGCTCTCAGATTATACTCCATGCT
CATCCGAACTGCGACTTCAGATACAGCAGCATCAGATGGGAGACGTGCTGGGAGTGCGTAACGCTCGCTG
CTTCCCGTGCATGTGCCTCACTCACTTCTGTGAACTTCCTCAAAGTCAGTTTTATCATCTACTTTTATGG
AATTGAAAACTGAGGAGACCTAACACCGTTTTTAAAAATTATCCTAAGGCCAGGCACGGTGGCTCGCACC
TGTAATCCCAGCACTTTGGGAGGTCAAGGCACCTGAGGTAAGGAGTTTGAGACCATGTGGTGAAACTCCG
TTTCTACTGAAAGTACAAAAATTAGCCAGGCGTGGTGGCGCATGCCTGTAATCCCAGCTACTCAGGAGGC
TGAGGCAGGAGAATCTCTTGAACCCGGGAGGCAGAGGTTGCTATGAGCTGAGATCACGCCACTGCACTCC
AGCCTGGGCCACAGAGTGAGACTCTTTCTCAAAAAAAAAAAAAAAAAAATCCAACAGTCCGAGCGCAGCA
GTCTCCCCCTAACCTCAAAAGCCTCCTCAGAAGTAACCTGGTATCAGTGGGCTGTGCAGATTCTTATAGT
CTTCTTTGCCTTTATATATGAAAATAACTTTGTTTTATGTTTGTTTTCACATAGGTGGAATTATACTGAC
TCTTACTCTGTGACTTGCTTTCTTCACATGACAGTAACTCCTGGACTGTTTCTGTAGCAGCCACACAAGT
CTCCTTTATATTGCCAATATCTATCTTTATCCCAAAGACAAGATGAATGTTCAAAAACGGTAATCCAAAC
TCACTTCTAAAAATGGGTGTGTGTTTAAAGAAGAGCTGCTCAGCTGAGGCAGTTTTGCTGCCGAGGGGAT
TTGAGCAATGTCTGGGGACGTGTGGTTGTCACAACTTGTGAGGGGGCTCCTGGCATTGAGGAGGTAGAAG
GCGGGAATGCACAGGTCAGGCCCCTGCAACAAGGAATTGTCAGGCCCAAAATGCCAATGGTGGCAACACT
GAGAAAATGTGATCGGTTTAAACCTGCTTATCTTAATGGTGTTTCAACAGCCTTATTAAGGGGCTTGACC
AATATGTAGTTTATCAACACCAGCCTTTGGTGTAGTGGGGTGAGTTGTGCTCCTGTTGGAGAAGCAGATT
CTAGGGCCCCACCCACAGAGTGCATACTTGAGAGGCAGAGGTGTGAACATTGCCCTTGAGCTCAGGAGCT
CCACAGCAGCCTGAGCAACATACTGAGAGCCCATCTCCTAAAAAACAAAAACAAAAAAGTGCTGAATAAT
AAGTCCATTTTAAACATATGATAAGATTTCAGATTTTCTGAGGAAAAAATTAATTAGACTTGGCTTCTTC
TCTGACTTGAAATTAGCAATACTTTTATACATCTCAAAGTGTGGTTTATGAAAGAGGCAAAATTGTCAAT
GACTACTTGTTGAATTTTGCTTCCTAACCATGTAATGGGGACAAGTGTTTCCGAAAACATGTTTTATATG
GTTAGTTTTCCTGCAGATGGGAATTGTTCGCTGAATTATACTTTGTCCACTTAGGCTTAGGTAGATATGG
ATTCTGTGAAAGTGGAAATCATTGAACAGTTTAAATACTGGATTAAAAGAAAAGGAACTATGGTAATTGC
AGATCTGTTAGATGGAATCTGTCGTGCCCTAATAAGAGTGATAAGGAAGACCATTAACTGTTGGAAGCAA
GTTGTTGGGTTCGAGGTTGGGAATTGGGCATGATTTGTTTCCTGTGTCACTGCTTAGGTTAATTTACTCA
GACTGCCATTTTTGTGTTGGAAGGAAATAAGGTCTAGGCCCTAAGCTTAGACTGTCTTCATGCCTAAAGA
AGTCCAACCATTCATTATTACTTCGCTAAAGGATGCATTAACATGAATATATATAATAAAGATAATGACT
CCTTTTTTCTCAACAAATTTTAAATTAACTTAAACATGCCTGATTTTCAGAAAGAATTAAAAGTAAAACC
ATACCTTTAATTTAGGACTGACTTCTGCATTTCGATTGTAGATTATTCTAATATGAAGAGTGCTATTTGC
ATGTTGGAATATCTTTTAATTCTTAATTTGTAAGTTACAAAGAAATAGGAGACTGAAATACTTTTTATCT
TACAAAATTCCAAATACCTTTTCAAGCAATTTATATCTTATTTCTATAATAGTTTTATCACCAGCCAATG
GGTATGCACTGTATGTCATAATATGGAAAGAAATTCTTTATCATTTCAGATATTAAGGATTTCAGATTTT
AAGAGTTATCAGTGAAACTGTGTTACAAGAATATTTTGTTCCTGAGACATCTCAGTATTATAGGTGGCTT
CAGACTAACAGGCACAGAATAGGATAGTGTGAAGTATCTGATTAGCTTTACTGTACTTGGTACCTTTAGG
ACGTTCAGCCAGAGGTGATTGGAGGGATTTGCACGTCTACCACTCGTGTGTGTATCTGTGAACCATATCT
CCTTTAGTGCCTGCTTTTGAACTTTATATAAACAGAATGATGCTGCCTGTTTATTTCTATAATTTTCTTC
TTTCCCTTAGCATGTGCTGTTCTTGGCCCCTTGCTTCTCCATGTGAATTTTGGAAAAGCTTCTGAAGTTC
CACCAAAAACCCTATTTGGAATTGTTACTGGTACTGCATTGAGTTTCTATGCCAGAGAACTGACATCTTT
GTGACATTGAATTTTTCTTGCTAAGATCCAAGGTCTTCCTTTATGCAGACCTTTCACATGTATTTTTAAC
AGTTTTGTAACACGAATCTTCTTTGCTGTCTTGTCATGTAATGCTTTGCCGGGGAGTGGTGGTCCATCTT
ACTCTGTAGAGGACATTTCTTTCTTTTTTTAGTGAAGTAAATGTAGGTGGTGCTTATGAGTTGTTTTAAT
TTCTAGTTTGTCTTTGTTTGCAGGGGGAAGTGATAATTTAAAATTGTGTTGTAAGAAAATTGGCCAATTT
TCACCATGGTATTTAATTTAGAAAAATATAAGTCACTTGCAGGTAATTTAGTCAGTTTGGGCATTTTGAT
ATTAAGTAAACAATTCCAGTATAATAACATAAATGCTTTCCTAGATGTTTACATATAATGCCATAATTAT
GTAAATAAATAAAATTCATTTAATTACATTAAATTTTTTTAATGTTTACATTAAAAAAAGGGGGGGAGAA
GCAACTAATTCAGCATTCGGGCACAAGTATTTTATATGGTCATGGTCAGAGAGCCCTCCAGGTATTCGAC
ATGGTAGCATGGCCACAGGCCCAGGACTGAATAGACTGGTTTGAGGAAAGAGAAGGTTAAACACACTGAT
TTATACCTGAGAAATACATTTTGGTGGGCCAAAAATAATTGAGTGTATCGGATGGAACTAACAAAAGCTT
TCTTTTACTAACCAGTTTTGCAGGAAAAACATGTTTTAAAATATGTAGATATGCATTATAACTTCTATTT
AAATACAGCTGAGGTTGAATTTCTCAGGACCCCTTTCAGCAAAGATGGCCCTTTGCTTCTGCTTTTCCTG
TCCGTCCTGGATGTTCTAATCCCAGCACCCGAAGGTTACTGCAAGCTGAGTGGCACCAAAGCAACACCGT
AGCCAATGAAAGTACATCATCATTGGTGTGCAGGTTCTGGTCAGTGTTACCAACATGAGTTAATATCTTT
GAAGTAAAAGCACATAGCGATGTCATGTTTTCCCAGTATTCTGTGGGTATTTTATGACCTGCAACTTAGG
AAAGCAAATTGTGAAAAAGAAAACTTGTTGTGTTCTCCTATAACAAGGTCACAAGGGAATCCTGCAGCGA
TTATGTTCTTCACTAGCTGTAAGAGAGCAATCTGTGCTCTTGGTTTTGACTCAGCTGGTCTTTATTGAAT
GACTATTGTGTGGCTCCCAGCTGTGGTAGGAATGTGAAGGACAGCGAGGCAGTGCCTGCCGTGGAGCGCC
TGCATGCTAACGGTGGACAGAGGAGAGCAGGCTCACCGTTACACTGCAAGGTGACCTTGGAAGGTGCAGG
GAGAGAAACACAAGGGAGGAACCACAGAGACTCCAGAACAACATCTTGCCCAGTCAAGAACTAGAACAGG
AATCCTAGAGAAAGGTGAATTTTGAAAGATGGGTATGATTTTGGCAGCTAGAGATGGGGGCGGGGAGGTT
GTTGGGTTTCTCAGAAGAAGGAAAAGGTCTGGAGGTGGGAAGGGGCTGGGTGAATTTGGTGACCAGCCAG
GAGTCTGATGTCACCAAGTCACAGGGATGAAAGAGAAAGGCAGGGGAAGTGAGTTTGCGCAGGCGGGCTG
AGGCCATGTAGGGGCAGGCTGGAGTAGGGGCCGGGAGCCTTAGGGGGTGACAGGAGAGGTTTTCCCACCT
TTTTTCTCTACTCCTTCCCCAGTCACCTTCCCCTGTCCCAGCGACATCACACATGCATTCTTTCTGTCTT
CCCCTTGCCTCTCCCTCCTCCACTTTGTCTTCTTCCCCAGCCCTCAGAATCAGTTCAGATGCCATTGTCT
CTGGAAGTACCCCAGAACCCAGCAGTTGGAACTCATCTCTCCTTGCTCTGTTCCCACAGCGGCTGCCTCA
CCAAGAGTCATTCCAGCACTCAGCAAGCTGGCTCACGTGATGGCTATTCCCACCTGCAGCACCCTTCACC
AGGCTGCAGGGCTTCTCACCCCAGTGGTCCCAGGAGAGTGGCTTGAAAGCAGATCATCCTCAACCTGTGT
TTGTTGAATGAATAAATAAGGGATCCCTATTTATAAGCACGTTTTTCTTGGAGATGGAGTCTTTCTCTAT
CTCCCAGGCTGGAGTGCAGTAGCGCGATCTTGGCTAACTGCAAGCTCCGCCTCCTGGGTTCACGCCATTC
TCCTGCCTTGGCCTCCCGAGTAGCTGGGACTAAAGGCACCCGCCACCATGCCCAGCTAATTTTTTGTATT
TTTAGTAGAGACGGGGTTTCACCATGTTAGCCAGGATGGTCTCGATCTCCTGACCTTGTGATCCGCCTGC
CTCGGCCTCCCAAAGTACTGGGATTACAGGCGTGAGCCACCACGCCTGGCCAACACCTTTTTTTTTTTTT
GTATATTTTGTATAAAGAGATATGCCATGGAGAGCAAAGTGGTGGAAATTCTAAAAGACTTTAATGATCA
GATGAAGAAATTGTTTAGAGAAGCATAATATACGTGCTCTGTCTTAGTTTGAGAATACTGACCACAGTTT
GGCTCTCCTAGCCCTTTGCCTGAGCAAAACCCACAACCCTTCCTCCACACCCAGACCATGTTCCAAGCCA
CAACCCATTGCTCCAGCTTTCCTAACCTGCCTGCGAATGTGCTGCCTGTTACACGAGGGAGCAGATACAG
TGAGGCAAGGACAACTAACAGCACCCAAAAGCAAGGGCAGTGGATTTCCCTCTGCTTTATTTAAAACTCC
CCTTCTTAGGAAAGTTCACTCGCATGATCTAAGCACATTCAATGGCTTTTCACACATGAGCAGAGTTTCA
TTCTTTTACATGAATCGAAATTGGGCTTAAAATCATTTTAGATACAGCCATCTTGGTTCCCATACTTTCT
ACTCATGAAAGATTTTATATCAGTGGTCAGGATGATAAAATCTGTACATCTTTATGTCTGAACCTGTTCA
TTTATATCCACATCCTTTTCTTAAATACTGATTTGTATATTTAGAAACTCAGTGCTTCATTAGTAAACTG
AGGGAGTTAGAGTAAAGGATCTCCAAGATCACTCTGCCAGCATTTGAGAAAGAACCTATTATTATATGAC
TATAGGAATTTATTTTTGTCCAAGAGCCAATGCCTAATCTTTAATGGTTTTGTGTTTAGACTATTTACCA
CTAGGTGGCGGTAAAAAAAATTATTCTTAGCAATGAGAAATGCTCATCAATATTTAAGTTTACTATAAAA
TAAAAATTTAGAGGACCAAAGTTTGAGGTGTCTGTTTGAAAACTACGTAACCATATACTGGCCTCTGTAT
GTTGTTTCTAGGACTTAACACTTATTTTATTCAGGGAAAAAACTCTTGTGGCACTTCATAGCCACTTATA
TTACAGAATTATGGAAAAACGTAAATTAAAATATGTAATTTTGTTTCATACACACACACACACACACACA
CACACACACAATGTGTACGTGTGTGTGTGTGTATGTGTATATATAGAGAGGAGAGAGAAAGGTTAGGTAC
ATTCAAGGTTTCCGGCATCCACTGAGGGTCTTGGAGTGTATCCCTTGCAGATAAGTGAGATACTACATTT
TCATTTTTCCAGGACATTATATGTTAAAGTCAGCTCGTGCAATGCAGACATTGGAAGTTTAGTCATGTGC
CTTATAATGACATGTTGATCAACAACAGCCTGCATGTACAATAGTGGTCTCATAAGATTATAATGGAGCT
GAAAAGTGTCTATGACCTAGGGACATCATAGGTGTTATAATGTAGTATAACACATTGTTCACATGTTTGT
GGTGATTCTAGGTAAACAAACATCCTGTGCTGCTAGTCATAGAAAAGGATAACACATATAATTATATATA
GTACATAATTACTTGATAATAATAAATGGCTGTGTTATTGGTTTATGTACTTAACATACTATACTTTTTG
TTATTTTAGAGTGTACTACTTATTTAAAAAAAAAAAAAGTTGGCCAGATGTGGTGGCTCATGCCTATAAT
CCCAGTGCTTTGGGAGACCAAGGCAGAAGGATCACTTTAGGCCAGGAGTTTGAGACCAGCCTGAGTGACA
TACTGAGACCTACCTCTACAAAAAATAAAATTAAATTTAAAAAATAAGCCTCAGCTTATTCAGTCTCAGC
TACTTGAGAGGCTGAGGCGGGAAGATTGCTTGAACCCACGAGTTGAAAGTTGCAGCGAACTGTGATTGCT
AGCCTGGGTGACAGAGCAGAGCAATACCCTGTCTCAAAAAAAAAAAAAAAAAAAAAAAAAGAGTTAACTG
TAAGACAGCTTTAGGCAGGTCCTTCAGGAGGGATTCATTCCAGAAGAAGACATTGTTATTACAGGAGATG
ACAGCTCCACATATGTTATTGTTATTGCCCCTGAAGACCTTCCAGTGGGACAAGATGTGGAGGTAGAAGA
CAGTGATATTAATTACCTGACCCTGTGTAGGCCTCAGCTAATGTGTGTGCTTCAGTTTTTCTCTTTAATA
TTTAAATTTTTATTGATACATAATATGCATACAGAAGTACATGCACAAAGCATTAAGATAAAGCTCAGTG
AATTATTACAAGGCAAATACATTTTTGTAACCACAAGATAGAATCAGCCTTGTTCATGCTTCTGACATCC
ACTTTCTTCCTTCTTCCTCTCCAAAGGTAGCCAAAATCTTAAGAGCTAATGTTATGGATAAACTTTGTCT
TTTTAGACAAGTGTTTTATTACAGAACATTTTAAACATATACAAAATAAACAAAATGGTGTAATAGACCT
GACACTCAGCCTCAACAACTACTCATCATGTGCTATTTCTGCTTCATCTATACTTCAGCTCATTCCCCAT
CCCTTTATTTTTTTATTTTTATAAGATAAGATGTATGTGCATTGACATGTATAATCTTTATTGTACCTTT
TTGACAAATCAGTACACCCATATAACTTTTTATCCTCTTAAGAATAGAATACCGGCTGGGCATGGTGGCT
CACACCTGTAATCCTAGCACTTTGGGAGGCTGAGGTGGGCAGATCACTTGAGGCCAGGAGTTCGAGACCA
GCCTGGCCAACATGGTGAATCCTTATCTCCACTAAATACACAAAAATTAGCTGGGCGTGGTGGCACACAC
CTGTAATCCCAGCTACTCTGGAGGCTGAGGCAGGAGAATCACTTGAACCTGGGAGGCAGAGGTTGCAGTG
AGCTAAGATTGTGCCATTGCACGCCAGCCTGGGTGACAGAGCTAGTAACAATCATTAATGTGCATATGAA
TCACCTGGAAATATTATTTTAAATCCAGATTTTAATGAAATAGGTCAGATTTTGATAAAATAGGTCAGGG
TTTTGTCTGAGAGTTTGTATTTCTAACAAAGTACAGATTCATAGAGCACATGCTAACTACAAGGTCTCTT
TTATCTAAAGTAAATAAAATTTGATGAATAAACTAAGGGAAATTGACCTTAGATGAAAGAACACATCAAA
ACACATGTGCAATATGCTATCTGTAGGAGCATTTGGTTAACGAGCATAAACCAACCAAGCAATTTTATAA
TTATTTTAATCCAAAGTTAGCGAAATCTCTATTATGGTTTCCATGGAAATGCTCCTTCAGGCTAAGAAAT
GTTAGTATCACCAGAGGGCACTGGTTAAAAACACCACAGATTTGGAAGTCGATGTCTTATAAAGTAAGTG
ACAGACCCCTCTTTACACCCATAAGTGGCTTTTACAGAGTTACCCAAAATTCAGTCATTTGTACACCAAG
TGTAGATAATTTTCATAGCTTCCTATTAAAATTATATTTTAACGCCCTTACAAAATTTAACTCAATTTTC
TTATAAAATGAAATAACATTTTTATATGACATTGTTATTCAGCTTAAACATACATACTTGTATTGGGTGC
TGTGTCCGTGTGTTGACCACCCAGTGTTCTTTGGGGTTCCACTCTTTGCACAGGCACCACAGCTGGAGAA
ACACAGACGCACAGACACAAGCTGGATCCTGCGTCTCTTCCTGCCTCCGGGCACAGGGCGCCAGTCAGCC
TGGTTGTGCGAATGGAGGTCGTGTTCCTTAGCACAGAGGGACTCATTCCAGGGCGCTCCCCTCAGAGGAG
GAAGAGACCGGAAGGTCGTTTTAGGGGGCACTTCCTGGGACATGGGCCAAGCCTCCTAAGGAGTGAGGGC
TCCAACCAGCAGGTACAGACCTCTCAGAAGTCAGGGATAAGGCGGCCATTCTGAACCTGGATCTCCACCA
CCCCTTGTCCCATCTCATCAAGACAGTTCTGAGGGGCTATTCTGGCACATGTCTGGACGTTAGAGGCTGG
AGGACAATGTGGGCTTTGATGGGATTCAGATAAGGTCTGTTCCAGGGAGGCAGGAGGTTGATGCTCAGCA
TCTGGGATGGTGTCCTTGTGGACAGTGAGTTGCCTCTTTGGGTACAAGTCCATTTTCACTACAGAAGAGA
CTAGAGCAGGGGTGTCCAATCTTTTGACTTTCCTGGGCTACATTGGAAGAATTGTCTTGGTCCACACATT
AAATACACTAACACTAGTGATAGTTGATGAGCTTAAAAAAAAAAAGCAAAAAATAATCTTATAATGTTTT
AAGAAAGTTTACAAATTTGTGTTGAGCTGTGCACTGACAAGCTTGGGCTGGAGAGTCTGAACGGCTCCCC
GATCCCCTCTCTGATTCTCTTTGGCAGCAAGGCCTTGAAGGGGCTGAGTCTCAGTGTCTTGGGGGAATTC
TGGAAAGCCTGTTTGGGAACCTGAGAAGACCTCTTGCCTGGAGCTTGAATGCTTGATTTGCTGTCCTGGC
CACATGCATGCTTGGCAGCCAGAGAGCGGGGTTTGCATTCACACCAGCACAACAGTGAGCCAGGGGTTGA
AGTGACCAGGACAAGTGTCTGGGTGGCAGTGTGCCTCAGTTTTTAACAAAAAAGTTTAAAAAGTAAAAAA
AAATTAAAAAATTAAAATAGAAAAGCTTATGATATAAGGATATAGTGAAAGAAGATATTTTTGTACCGCT
GTACAATGTGTTTTAGGCAAAGCATTGATTGTAATAGTCCAAAAGTTCAACAAAAATAAAAGGTTTATCA
AATTTGAAAGTTGCGGTAAGCTAAGAATAGTTTATTGCTGAAGAAAGAAACAAGTTTTTAAGAATTTAGT
GTAGCCTAAGTGTACAGTGTCTGTGAAGTCTACAGTGGTGCAGAGTCATGTCTTCGGCCTTCATATTCAC
TCCCCATTTACTCACCCAGAGCAACTTCCAGTTCTTCAGGCTCCATTCATGGGAAGTGTCCTATACAGGT
GTACCATTTTTTCAGTGTTATACCATAGTTTTACTGTACCTTTTCTATGTTTAGATAGGTTTAGATCACA
ATGTTACAGTATTCTGTACAGTCCCATGCTGCACAGGTTTGTAGCCCAGGAGCAATAGGCTCTCCCATGT
AGCCTAGGTGTGCAGCAGGCTGAGCCATCTGGGTTCATGTAAGTACACTCTGTGATGTTTGCACGCAAAG
AAACTGCCTAACGAAGCATTTCTCAGAATGTATCCCAGTTGCTAAGTGATACATGACTGTAATATCTAGG
GAACTATTTGTGGCTATTTTAAACATTACTTAAGAATTATTTACAATCTCTCATCTCACCCTCTGAATCT
ATCAAATGTCAAGACGGAGCTCATACAGTATTGGGAAGTCACCCTCCGTGAGCTGGTCCCAGGCCCAGGG
TGTGAGTGCTTCTGGGAGGCTGTGGGTTAGGGGAAAGCCTGGGCATGGTTTTGTTTAACATCTACAATCT
TAACTGACCCTTAGCTGTTACTGCTCAAAACTGGTTTCCTTCAATTACATACTACATTACTTTCCATCTC
AACCAGGTTTGTAGCGCCTTGCGCTCTAATTTTTTAAGGGTGGATTGTGATATACAATAACAGTAGCCAC
CTGTTTATGGGTGGCTTAATTGAAACTTTGCTGAATTGAATTTGATTAAAAACTGAGCCTGGGATTTTCA
GTGAGATGCCACACTTAGAGAACATAGTGCCTCACACATTGTAAGCACTTGGTAAATTGTGGTTGTCACA
ATTGCTAGAATTCCTTCAAGAAGTCAATTCTCACAAGTGAGGGGCATAGGAGAGCTCCCCAGGCAGAGGG
GATTGGACATACTGAAGTCCTGATGGCCCAAAAAGCCTGTGGCCCTCCAGGGTCGCAAACAGGCTTGTAA
GGAGAAGTCAAGCCTGGTGGCGGGTGTGGGGTGTGCTGGTGAGCCAGGCTGCTTCACTTTGAGAAGTTTG
GGCTTCACAGTAAGAGTAACTGGAAGCTGTAAGGGTCTCAGACTGAGAGCATTCAGATTTGCATTGTTAA
AAACTATCCCAACAGTCTCATGGACAGTGGATACAGGGAGGTTCATTATAAGGTTTGGCCATAGAGTAAA
TAACTAGCCTTGGGCAGGCTGCTTAAACTGTCTTGATTTCCTCCTCTTTAACCCAGATTCAGTAACAGTA
CCTACCCGTAGGCTGTGAGGATTAAATGAGTTGCTATTTGTAATTGGCTTAGAACAGCCCCTGGTGTGTA
GTAAATGCTTTAAATGAGTGTTAAATAGAATGCAAAAGTCTGGTTTCTGAGCAGAAGGTTGCATTCTTCT
GACACTGCCCTCCCCTCACCTAGCATCAGACTGACCTGCTCTGTGCAAACCCAGACTGCTGCCGACTTGG
AGCCCACCTACTTTCCAAACCAAGTCCTCCCTTCTCTGCCTGGGTCTGACTTGGACACCTCCGCGTGGAT
TTGACATATGGCTGGCCAGACCTGGCCGGGCTTTCCCTTTTCCCAGAACACCTGCACTGCACCCCACTGG
GGATTCAGGCTCCATCCTGGCTTGCTAAGAGCACCAAGTGCTCTCCCTGCTGTGCAGGCCCCACCCTCCG
CCTTCGCAAGTTCAGTGGAGTCCAGCAGTAAGCGCAGCTGGCAGTAAGCACTTCCACAGATGGCACGCTG
GCTTGTACCTGAGCTCCCTGACTCTGCCCACCCAGCCATGGCTGCTCCCTCTCTCTCCCAGATTGCACTT
GGTGTTGGAAGAGAACACCACCAACATGGAGACGAACTCTCTTGGGGCTATGGTCAGCTCCTATTTGCTT
TCTGTATTTGCGGCAGATTTGAACTGAAAGAGGGAAAAAACAATAACAAGCAAGTGGCCCCATCCTTCTC
TCTACCTTAAGCAAGGTTCACTCTAACTCTCTAACTCTGTAAGGTTCCTGTTTTTACCCCTTTCCAGTGG
ATTCATTTTTTTGTAAATCATGTAGTACATATTACTGGGGGTTTTTTGTTGTTGTTTTTATTAATCATGA
ACGTCTTCTACTGAGTAGTAATGTTTTAAAGCTACTTTGTTAACTTGCAATTCTAATTTGAAAACTGTAG
ATAGGATGTCATGTAGAAATGGCAGGTTTCCACTTACATGACTTAGGAGTGTTGCCTGGATTCTCTTTTA
AGGATACGGAGGCAGGGGTGAGCTCACAGGGAGAAAGTACATGATCTGCACAAAAGGATGCATCCCAGAT
TCAGGGTAGAGTTTCCCCCAGGGGTAGGGCTGTTGAATCATGATGGGGAAGGGCAGAGGGCATAGAAGGC
TTCTTCTGTATTTCTAATGTTTACTGATTAAGCTTGAAGGTTTGTGGAATTTGACATAATATTTTGTATC
TTTTTGGATGTGTGAGATACTTTATGATGAAATTGAAATGACCACCGTGATAATGGTAACCACTCAGAGT
GTCACTGGTATGTGTGCCACGTGCTTGTCGTCCCGGGTCTATGGAGCCTGCCTGCCCATCACTGAGGCAC
CCTTAGCCTCCACCCTGGATTAGACTGTCATCTCCAAGTATTTGTGTATGAAGTTACATACATACATTTA
TATGCACAGATGCAAATTCATTTTATCTTGTAATGTGTTATCACAAAATGCAGTATAGGACTTTTTTAAA
GATCAAATATTTCCAAATAAAACTTTTTCAAAAAATATTTTTCCTGGTAGTAAAAGAAGCACATACATGT
ATGGAAGGCTAGGAAATTGTACTTAAGTAGAAAGAAGAAGAAAAAAAATATATATATATAATGTTAATAT
AAATGTTAGCAGTCTCTAGGGTGGTACTATTATGGGTCTCTAGCACCACCCTAGAAACTGCTAACATTTG
GTGATAGCTGTGATCTAAATATATCCCCTAAAATTCATGTGTTGAAACTTAATCACCCATGTGATAATAT
TAAGAGGGGCTTTGGGGGTGATTAAGTCATGAGGGCAGAGCTCTCATGAATGAGACTGGTGATTTTATAA
GAGGTGGAAGAGAGTGCCCTGGGCCCTTTTTGCCCTTCTGTCCCTTTCACCATGTGAAGACACAGCATTC
ATAGTGTCCGTTTCCTCTAGAGGGCACAGCAACAGGGCATCACCTTGGAAGCCTTCAGCAAACACTGAAC
CTGCCAGCTCCTTGGTTCTGGACTTCCCAGCCTCCAGAACTGAGACATAAAGTTTTGCTCTTATAAATGA
CCCAGTCTGAGGTACTTTGCCATAGCAGCACAGACTAAGACAGTGATGTTTTCTTCCATTGTTTTTCCAT
TCTCATTTATGGATTATTTTTATATTGATGAGATCATTTCTGAGTATAACATTTTACATCTTTCTTCATT
TAGTAGTATAATATAACCATTTTAAATACTATTAAAATTATTCATAAACTTTTCTTTTCCTTGGAGACAG
GGTCTCACTCTCAACTCCCAGCCTGGAGTGCAGAGCCTCAATTTCTGGAGCTCGAGTGATCCTCCTGCCT
CAGCCTCCTGAGTAGCTGGGACCACAGATGTGCACCACCATGCCCAGCTAATTTTTTTGTATTTTTTATA
GAGGTGGGATTTCTCTGTGTTGCCCAGGCTAGTCTCGAACTCCTGGGCTCAAGTGATCTGCCCGTCTCGG
CCTTCCAAAGTGCTGTGATTATAGGTGTGAGCCACCGTGCCTGGCCCCTTTGTAAACATTTTAGTGGCTT
ACAGTGAGCCACTATATAAGCATATTAATCATTCTCTGCTGTTAGACAATTTTTACTATATGAGAAATAA
CCACTATAGTGAACATCTTCGAGCATAGTTCCTGTCATCATATTTGCATTTATTTCCTTAAATGTATGCC
ACATATATCAAAGGTTCTGAATATTTTGAAGACATTTAATATATTTTTTTCAAGTTGCTTTCTATAAGGC
AGGGCCTCTGTTAGCCCGTAATCGGTCTTTGTTCAGGTGACAAACCTCTAGGCAGACACACCCCTGGAAG
TCCAAGCTTCATGATGGGTTCCGGGCTGTGTTGCCTCTGCTCACACTTTGGGCGCCTCGGTGCCATTCAC
AACTGCACTCTGTATGGAAGACCCTATTTCTATTCCTCCAGGAGCTCACACAGAGTACATGTTTCATAGA
ACCCCATACAGCTTAGCTATTCATGTCTGTAAAAAACTTGAATTCATTAAGTGAAAGTGCCATACAGGTG
TTTTACCCTACCCTTTTTGATTATGGAGGATGAACATTCATTTATGTGTTTCTTAGCTATTTGTATTTTA
TGAATTATCTATTCATATTCATTGTTATGCTCTTAGTGTTTGTCGTATATTTTGTACATTAATTTGATAA
TATAGAATCTGTACTGTTTGTTGTTAACATTCTTCTTAGTTTGTGCTCATATTATTTTTCTGGTAAACAC
AAGATTTTATTTTTATTGAGCACAGCCAAGCCTCTTCCCTGATGATTTACTCCCAGGATTTTCAGTTCTC
AGAGTTCTTTCTCATTCAGATACTTGAAAAATGTTCATGTTTTCTTCTTGTGTATTATTTCATTTTTACA
TTTATCTCTTTGATTTACATTAAACTGGAGTATATTTTGATGAATAATACCCGGTGAGAATCTTTTTTTC
TTTTTACAGATAGTTTTTGAGGCACCATTTATTACATAATCTGTCCTTTCCCTACTGATTTGTGATGTGT
TCTCTATCTTATATTAAATTCTAATATCTGGATCCTTTTGTAGTTCATGAGCGTGATGATTGGGTGTTTC
ACGCATGTGTGTGCAATGTGCCACCCTTGAACCTTGTATGACATCGGCACGTTACCCATCTGACCTCAAA
AAAAAAGCAAAGAAAAATTATCATCTCTGTTAAACATATTTTACTGAGTTTAAAAACAATAAAGATTCCA
TTCTTAATATAGGTAAAGCACTGCTCATTGATGATATATATTTTTGTTTCACTGCTAATGTTTGTTTAAA
TTGACTTCTTTTTAATGTGTTAACTTTAATGCTAACATTTCTCTTTTACACTTTGAATCAAAGTAAATTG
GGTACTTTGACAAACAGATTTTTTTGTTTCATAGAGACCACCCCCATGTACAAATAAATTGTAGGCATTG
TACATAAAAGGCACTTGCATTTACTTTTAAAGAAGTTAACTTTTTCTGTTTATGTTTTTTAGGGTGTATT
ATCTCATATACTTTCAGCACTCAGATTTGATAAAGAAGTTGCTCTTTTCACTCAAGGTCTTGGGTATGCA
CCTATAGATTACTATCCTGGTTTGCTTAAAAATGTGAGTATTTAAAATTTATCACTTTTGAAATGTTTAA
TGCTGAATGTGCCATCAGCAAAAAGAGTAAATGGAAATATTTCAGTCCTCCAGAAGAGATGTTTAACTTT
TCTTTGTTTATCTCTTCTTACCTTGGGCAGACTTATGGCCATGTACGGAAGAAATGTGAGATGGGAAGTT
ATGAGAAAGAAGGAAACCAGGAAGGGTTTTCCTAAACCAACCAATCAGCCTCTCTTTCTAGGGACACATC
TCACTTATTCACTCAGAGATGTTTGGGAGAAGAGCTGTTCTTAGCTATTATAAACACAGTCTTGTACTGT
TGAAAGAATCTTGTATTTCAAATAACCTGATTGGAATTTTCTGTTAAAGCAAAACAGAAAATTCAGTACA
TAGTTTTAAATATTTACCTCTTAATATTAAAGCATTGTTTTCTTCTGGCTTAAATTATAGCCAAATGTGA
GAAATTTTAACTTACAAACTTGATGTAACTCTTCTAAAAAATGTTATGAGATTTTTTAATGTTTGTTAAC
CATTCCGGTGTTTTGGAAGCTTTGTACAATAACAACTTTTTTTTTTTTTTCAAATGAACTGAATTTTAAA
TGAAGAAGTAAACTAACTTTTCTTTAAATGGATTTGGTTTTAATTCTTAGGAAATTAATGACCTGTCTAT
TGTTCATTGCTTAAATAGGAATGCAGAATTATAGACATTAAACATAAAATCCCAATTATTAGTAAATGTG
ACATGGCACTGCTTCCTTTTCACTCTGACAGAGTGAAACATGAGAAGATGGGGGTTGGGGGAATCTCAAC
GGAAATATCACTCACACCAAGAAAAATAGAACTGATGTAATCCTGTTTGCAGCGTGAGTTAACCTGCAAC
TGATTTTGTTTTACAGATGGTTTTATCATTAGCGTCTGAACTCAGAGAGAATCATCTTAATGGATTTAAC
ACTCAAAGGCGGTAGGTGTTAAACTAAACATCCTTCTTCTCAGGTTTCAAAATGTATCAGTTTGGTTATG
AGAGGAAAATTTTACAATTCATAGGAAATGGATGTTCAGTTATGGTTGTATTTTATATAGAAAGATTATT
TTAGTGGAATACATAGCAAATTGGTGAGTTTTTTCAAACTCTTTTAAAAATCACTATTTTCTCAACTCTC
ACAGAGCAGTAAGTAAATCATACAATGTCTTTTGTGGGCCCATTAGGTAGAAAGCCCTACATACACAGTA
GGGAGCATAAAAGAAAGAGCAGTACTTAGTTTTACCCTGGGATTGCTCACTCTGCAAGAGGAGAATGTGC
ACAGATGCAAGTGTATGCTCTCACACTAGCCTGCCCTGGCTCTGCCTTCTTCTGCTCCCAACACACACAC
ACACACACACACACACACACACACACACACACGATATTTACAAGGCAACATTTGATGTGAACATATGCCC
TATTTTAAGAAACATGATATTCCAAAATCTAGACTAAACAAATCAAAGAGATTCAGACAAATTAAAAGGC
AATTAATCACATTACAAATGTTTCTTCACTCTGTAAAAGAATCTCAGTTTTCATTGGTGGGCATTTCATT
TTTGACATATCATTAACCAAGAGAATCTGGGACTGAGGGACTTGGTGATTTTTTTTTTTTTTTGTAAAGC
TATTGCTAATTCAAGCTCTATCAGAAATTGTCTTTGTCCTCAACCTTTAGGAATCCTTTGCACCTAACCC
TTAAACTATATAATTATTAGAGGAAAACATTGGAGATAATCTTCATTATGTTGGATTTGGCAATGTAAGA
AGAACCATAGGAGACATGGAGAGTGTTTAGATTTATTTATTTACATGTCAGGTAAAGACTTGGATTTGGT
TGTGAGGTGAATGTTGACTGATGGATCTTTTGAATTATGTAATTATAAATTTATTCAAAAAGACCTTAAA
AATCAGTCAGTGCAGTGTGTATGTACTCCCTACACCAAACTCCCAAAATATGCATCACAGTTGATTTTTT
AAAAAGCAGCTGACAATAAACAAAATAATTTTTAAAATCTGTTAACAAATAGAAACAAAGGATCAGAATG
CAAAATATAGAAATGACTTGCAAAAATAAATCATAAAGAGGCAAACACTCCGTTATAGAAATGAGCAAGG
AATATAAGCAGGTCATTACTAGAAGATGAAAACTGAATTAATAATAACATTAAAGGCGTTCTTCAGGAAT
GACATCAGCAAGATGGCAGAATAGGAAGTCCTAGACCTTATTTTCCAGTAGAGACATTGACTTAACTACA
GCATACATCCCAAAAAATTATAGTGAGAACTACAGAACCAATTAAGAAGTCACAGTACTGCAGGCAAGCT
CAAAGTCTAGAATAGCCACATTGAAATTGATTAAACAATCTGTTTCATGTCAACTGTGATAGCCCTTTTC
CAGGTCTGGCAGCTTGGTGAGATTGGTAGGAAAAACCCAGCTTGTGGCTTCTCTTCCTTGGGAGGGAAAA
AGAAGAGTGGAATGTGCATCCAACATTCTCACTTTTTAGGAAGCAGACTGAGGAACTGATTTCTGTCTCT
ACTGACCGGGAGCACTGATGAGGAAATCGCATACAGTAATCCCTCCTTATCTATAGGGAATATATTTCAA
GACACCCAGTGGATGTCTGAAACTGTGGATAGTACCAAACCTGATTGCCACCAATCATAACACATTTCTG
TATTCCACTCACAACTTTAATGTCTTTTCCATCGTAACTAAGCACTTATCATATATGATGGCTGTAATTT
TTGCCATGTGAGATGCAACGACAAAACTAACAGAAATTTTTTTCCTTCTTTACAATTTCACAGATAGATG
ATTTGTTTTTACTATAGATCTCAGCAACCTCAGCATATGATTTTTTTCTTTCCTTATTAAGTTGAAAACT
TTCACCTTTTCACTTAAAGAAAGCACTTTACAACTTCTCTGTGGCATATCCAAATTGCCAGCCCCAGTGC
TCTTGGACTTTGAGGTCATTATTAAGTATTAATAAAATAAGAGTTACTTGAACACAAGCACAGTGATACT
GTGACAGTTGATCTGACAACCAGTATGGCTACTAAGTGACTAACGAGCAGGTAGTGTATATATTATGGAT
ACCCTGGGTAAAGGGATGATTCATGTTCCAGATGGGACAGAGTGGGACAGCATGAGATTTCATCATGCTA
CTCAGAACAGTACACAATTTAGGCCAGGTGCGGTGGCACATGCCTTTAATCCCAGCACTTTGGGAGGCTG
AAGTAGGTAGATCACCCGATTTCAGGAGTTCAAGACGAGCCTGGCCAACATGTCAAAACCGCATCTCTAC
TAAAAGTACAAATTAGCTGGGCGTGGTGGCTTATGCCTGTAGTCCCAGCTACTCGGGAGGCTGAGGCAGG
AGAATCACATGAACCTGGGAGGTGGAGGTTGCAGTGAGCTGATATTGCATCACTGCACTCCAGCCTGGGC
AACAGAGCAAGGTGCCATCTCGAACAAAACAGTGCACAATTTAAAACCTTTGAATTATTTCTGGAATTTT
TTCATTTAATATTTTTGGACCACGGGTGACCATGGCTAGCTCAAACTACAAAAAGTAAAACTGCAGATAA
AGGAAGACTACTTTTGATGCCTTGGGGCCATGGAGAACAAAAGAAAGCTTAGTGGTTTGTGGTAGCACCA
GAGAATTGACAGTGCTGCCGATGGAGGCTGGCATAGCTCAACAGAATTGGGAGGAAGCACTTAGCTCATG
ACCTGTTCTTTGGGAGGGGAAGAGAAGCCTCTGGCTTCTCAGGGGCCTACTAGAGGGACTGGTATCTGTC
TTGCCTGACTCACAGCACTGACAGAGAAATGGCGTAGTTTGGAACAAGAGAGCTCAGCAGCTTGTTGCAG
CACTGGAGAATCTGCAATACCACAAACGGACACTAGAGGGGAGGGGAAGATTAGGAGCTCTTGAAAAACC
AGTAAAGCCCTCTAATTTGGAAATCACACGCATAAGCCCAGAGAAGATATATCCCTGGAAAAGGTTTGAG
GGACCTCCAGAATGTCTAGCCGGGTCCACTGGTGAAAGCCTTCCTTTGTACCATGTTAGTCGATAAAGAC
TGAGAGAAGTAGATGTTTCTTTAAATGTACAAAATACAACAAAAATAACAAGACACAAAGTAAACCAGGG
AAACGTGGCATAATCAAGGGATGAAAATAAATCTACAGAAACAGACCCTAAAGAAGCAGAGATCTATGCA
TTACCTGACTAAGTATTTGAAATAATTGCCTTAAGGGATCTCAAGGCACTACAAGAGAACACAGATAAAC
AACTAAATGAAATCAGGAAAACAATATGTGAGCAAAATGAGAATATCAACAAAGAGATGGAAACTAGAAA
AGAACCAAACAGATATTCTGGAGCTAAAGAACCACAATAAGTGAATTGAAAAACTTACTAGAGGGGTTCA
GCAGTAGACTTGATCAAACAGAAGAAAGGGTCAGAAAACTTAAACACAAATCATTTGAAATTATTAAGTC
AGAGAAACAAAAAGAAAAAAGAATGAAGGAAAGTCAAGAAAGCCTAAGACACTTGTGGAACAACATCAAA
TGGAACAATACACACATTTTGTGAGTCCTAGAATAAGAGAGAAAGGGGCAGAGAACTTATTTGAAGAAAT
AATGGTGAAAACTTCCCAAATATTAGGAAGGAATTGGATATCCTAATTTCAGAAGCTCAGCAGACTCCAA
AGAATGAACCCAAAGAGGCCTACAACAACATACACAAAAATCGGATTGTCAGAAGTTAAAGACATAATCT
TGAAAGCAGCAAGAAAAAAGTGACTTGTCATGTACAAGGAAGGTTCCAGAAGATTATCAGTGGATTTCTC
AGCAGAAATATTACAGGCCAGAAAGGAGTGGGATGATACATTCAAAGAGCCAGGGAGGAAACAAAACAAA
ACAAAAAACCCTTCCAATCAAGAATACTGCATCTGGGCTAAATCATCCTTCAAAAAATGAAGAAAAAAAG
ACATTCCCAGATAAACAAAAGCTGAAGGAGTTCATCACCATTAGACCTGCCTTACAAGAATTGCTAAAGG
GAGTCCTTCAAGTTGAAATGAAAGAATATTAGCAACATGAAAGCATGCAAAAATATAAAGCCTTCTGGTA
AATGTAAATATACAGACAAACACAAAATCTTATAATACCGTGTTGTATAGTACAGTTGTCCCTCAGTATC
CAGAGAGGATTGGTTCCAGGACTTCCCACACATACCAAAATCTATAGATGCTCCAGTCCCATAGTAAACT
GTATGGAACTTACAGATACAAACGTTCAATCTTTGGGTTCTACATCCTGCAAATACTATATTTTTGATCC
ATGATTTAGTTGAATCCATGGATACAGAACCCATAGATATAGAGGGCTGAGTGTACATAAATCACTTTTT
AATTCTGGTATAGAACTTAAAAGATAAAAGTGTAAAAATAACTATTAAACTGTGTTGATAGATACAAAAT
AGAAAAGGATGTAATTTGTGATATTGATAACATAAACTGGGGAGGGAAATGTAAATGAGTAGAGTTTTAG
TATGTGATTGAAGTTATCTCGTTAGGTTGTTATAACTCTAAGATGTTTAATGTAATGCCCATGGTAACAA
CAAGAAAATACCTGTAGGAGATGTACAATGGAAAATGAGAAGGAAAGCATGTCACTATGAAAAAAATCAA
TGAAACCCAAAGGAAGGGAATAAGAGAAAATCGTGAACAGAATAACTACTAGGTATATGGAAAACAACAA
AATGGCAATAGTAAGTCCTTCTCTATCAGTAATTACTTTAGGTGAACATGAATTAAACACCCCATTCAAA
AGATGTGGAATAGCAAAATAGATCAGAAAACAAGATCCAACTATATTCTAGCTACAAGAGACTCACTTGA
GATACATAGGCTGAAAATGAAAAGATGGAAAAATGGCAACCAAAATAGAGCAGGCATAGCCATACTTATA
TCAAACTATCAAAAGAGACAAAGAAAAAGATTATATAATAATAAGAGTGTCAATTCAGGGAGATACAACA
ATTACAATAATATATGCACTTAACACAGCATCCAAATATATGAAGCAGATACCAATAGAATTGAAGGGAG
AAAGAGACAGTAACACAATAATAATAGATTTCACCACCCCACTTTTAATAACATATTGATTAACCAGACT
GAAGACCAATAAAAAAATAGAACTTGAATAATGCTGTGGACCAATTGGACCTAATAGGCATATAGAGAAC
ATTCTACCCAATAACAGCAGAATATACACTCTTCTCAAGCACATATAGAACATTCTGTAGGAAAGACCAC
ATGTTAGGCCACAGAACAAGTCTTAACAAATTTAAGAAGACTGAAATCATAGTAACTATTTTTTCTGACC
ACAAGGGAATAGAAATCAGTAACAAAAAGAAAATTGGAAAATTCACAAATATGTGGAAATTAACCCACTC
TTAAACAACCAGTGAGTCAAAGAACAAATTACAAGAAACACAGACAATAACTTGAGACAAATGAAAATGA
AAACCCAACATATCACAACCTATGGGATGCAGCAAAAGCAGTTCTAAGCAGGAAGTTTATAGTGGTAAAT
GCCTACATTAAAAAAGAAGAAAGTGTACATGCTTTTGAAGGAAGAAAAAAAAAGAAAATAAGAAGGATCT
CAAATCAGAAACCTAACTTTACACCTCAAGCAACTAGAAAAAGAACAAACTAAACCTAAAGTTAAAAGGG
AGAGACTAATAAAGATTAGAACAGAAATAAATAAAATAGAGAATAGAAGAAAAAACTAAGAGTTGGGTTT
TTGTAAAGATCAGCAAAATTGACAATTCCTTAGACTAAGAAAAAAAAAGAGAAGACTTAATAGCAAAAAT
CAGGAATAAAGAATAGAAATTAAAACTGATGCCACAAAAATTAAAAAGATTGAGACTACTATGAACAATT
ATATACCAACAAATTGAATAACCTAGAATAAATAAATGTCTAAAAATATACAGCCTACACGACTGAATAA
TGAAGAAATAAAAACATCTGAACAGACCTACAACTAGTAAAGAGATTGAATTGGTTATTCAGAACCTTCC
AACAAAGAAAAAAACCAGGACCAGATGGCTTCACTGGAGAATTCTATCAGACATTTAAAGAAGAATTAAC
ACCAGTCTTTCTTAAAGTCTTCCAAAATATTGAAGAAGAGGGGACATTTCCAAACTCATTTTGTGAGCAA
GCATTACCCTGACACGAAAATCAGGCAATGAAACTACATGAAAACAGATTAATATCCTTCGTGAATCATG
ATGGAAGGATTCTCAGCAAAAAGGGAATTCAACAATACGTTAAAAAGATATGTATAAACACCATGACTGA
ATGAAAATTATTCCTTGAATACAAGGATGGCTCAACATACAAAAATCACTATATTGCACCACATTAATAA
AATGATGAACAAAAACCACATGGATCATCTTAATGCAGAAATGGCATATGACAAAACTCAATACTCACTT
ATGGTAAAACCTTAAACAAATTAGGAATAGAAGGAAATGACCTCAATGTAGTAAAGTAGTAAAGGCCATA
TTTGAAAAATCCATAGCTAACATCATACTCAGTGGTGAAAAAGGGAATGTTTTCCCGCTGAGATCAGGAA
CAAGGCAAGAATATCTTCATCCTCTCTTACCACTTGTATTTAATATAGTATCAGAAGTCCTTGTCAGAGG
AATTGGGCAAGAAAAAGAAATAAAGGTCAACAGATGCTCCAAATTCTTTCCATCCAAATTGGAAAGGAAG
GAATAAAATTGTCTCTGTTCACAGATGACATGATTTTATATGTTAATAATCCTAAAAGATTTCACACAAA
AAAATTGTTAGAACTAATAAACTAATTCAACAAAGTTGCAGTATACAAAATCAACACACAAAACCAGTTG
CATTGCTATACATTAATAAGAACAACCCAAAAAGGAAGTTTAAAAACAATCCTGCTTACAATAGCACCAA
AAAGAATAAAATATTTAGGAATAAGCAACTAAGGAGGTGAAAGATTTGCACAGTGAAAGCTATACATTGC
TAAAAGAAATTAAATAAGACATAAATAAATAAAAAGATATCCCATGTTCATAGATGGGTATGGTTTTTTT
GGGGTTTGTTTGTTTGTTTATTTTTGTTTTGAGATGGAGTTTCGCTCTTGTTGCCCAGGCTGGCATGCGA
TGGCGCAATCTCGGCTCACTTTAACCTCCTCCTCCTGGATTCAAGCGATTCTCCTGCCTCAGCCTCCCGA
GTAGCTGGGATTACAGGTGCACGCCACCATGCCCAGATGATTTTGTATTTTTAGTAGAGATGGGCTTTCT
CCGTGTTGGTCATGCTGGTCTCGAACTCCTGACCTCAGGTGATCCACTTGCCTTGGCCTCCCAAAGTTCT
GGGATTACAGGTGTAAGCCACTGCACCCAGCCTATGCTTAGTATTTTAAATGCCCGTACTACCCAAAGCA
ATCTTCAGGTTCCAGTGTTATTTCTATCAAAATGTCAGTGGCGTTTGGTTTTTCTTTTATTTTTTACAGA
AATAGAAAAAGAATCCTTTAATGTATATAGAATCTCAAAGTACCCCAAGTAACCAAAACAATTTTGAGAA
AGAAAAACAAGGTGGAAGCCTCATACTTCCTTTTTTCAAAGCATATTACAAAGCCACAGTAATCAAAACA
GTGTGGTACTGGCATAAAGGCAGACATATTGACCATTAAAACAGAATAGAGAGCCCAGAAGTAAGGCTTC
ACATGGAAGATCAAATGGTCTTTGACAAGGGTATCCTGTCTATAAGATTGGGAAAAGATTGTCTCTTCAA
TAATTGGTGTTGGGAAAACTGGATATACACATTGAGAAAACAAATTTGAACCCTTACCTTTCATTATATA
TAAAAACTAACTCAAAATAGATGAAAGACCTAAACGTAAGACCTGAAACTGTAGGAGAAAAGTGTCATGG
CATTGGACTTCGCAATGATCTTGGATATGACACCAAAAGCACAGGCAAGAAAAGCAAGAATAGACAAATA
GGACTGCATGAAATTTTTAAGTGTCTGTGCAACAAAGGAAGCAATCAACAGATTAAAAAGCAGCCTATGT
AATGGGAGAAAATATTTTCAAACCTTAAATCTGGATAAGTGATTAATATCCAGAATATACAGAGAATTCC
TGCAACTCAACAACAGCAAAATAATCCAGTTTAAAAATAGGTCAAAGACTTGAATAGACATTTCTCCAAA
GAATATATACAAATGACCAACAAGCATATGAAAAGATGCCCAACATCACCAGTTATCAGGGACATGATAA
TCAGAACCATAATAAGATACCAGTTCACACCTATTAGAATGGCCACTATCAAAAGAAGAAAGAGAAAATG
ACAAATATTGATGAGGATGTGGAGTACTTGGAACCCTTGTACACTGTTGCTAGGAATGTTAAACTAGTGC
AGTCACTATGGAAAACAGTATGGAGTTTCCTTAAAAAATTAAAAATAGAATTACCAAATGATCCAGCAAT
CCCATATTTGTGTATATTTCCAAATAATTGGGAACAAAATCTCTAAGAGATATTTGCACACCCATGTTCA
TTGAGCATTATTTACAAGAGCCAAGAGGTGGAAGTAACCTATGTGTCCATCAGCGGACGAATGAATAAAG
AAAATATGGTATATATACACATTGGAAGATTATTCCACCTTAGAAAAGAAGGAAGTCCTGTCATATGCTA
CAGTGTGGATGAAACTTAAGGACATTATGCAAAGTGAAGTAAACTAGTGACAGAAGGCAAATACTGCATG
ATTACTTACACTTATGTGTGGTATCTAAATTAGTCAAACTTGTAGAAACAGAAGCTAAGGGCAGGTGCAG
TGGCTCACACCTGTAATCCCAGCACATTGGGAGGCCAAGGCAGGCGGATCACCGGAGGTCAGGAGTTTGA
GACCAGCCTGGCCAACACGGCGAAACTCCATCTCTACTAAAAATACAAAAATTAGATGGGTGTGGTGGCG
GGTGCCTGTAATCCTGGCTACCCAGGAGGCTGAGGCAGGAGAATCGCTTGAACCTGGGAAGCGGAGGTTG
CAGTGAGCTGAGATCGTGCCATTGCACTCCAGCCTGGGTGACAGAGCAAGATTATGTCTCAAAAAAAAAA
AAAAAAAGAAAAGAAAAGAAGCAGAAAGTAGAATAGTGGTTACCTTGGGATGGGAGGAAGAGGGAAATGG
GAGTTGTTGTTCAATGGGTATAGAGTTTTGGTTTTGCAAGATATGAAAGTTTTAGAGATCTGTTGCATAT
AAGGACATATAGTTAACACTACTGTACTGTATAGTTAAAAATGGTTAAAATGGTAAATTGTATGTTAATG
TGTTTTTCACCACAATTTAAAACTGGTCTATGAGCTTTTCTGTGCATTTTCAGGAAAGTGTAAACTAACA
ACATCAGTTAGACATCCATTAGGTTATGGTGAGCTTTAGGAGGTTGCTGATGGGAACATAAATAGGCACA
ACCACTTTGCAGAACAATTTGGTCTTCCTGGAATTTGGGATTCCTTGTTAGTCTCTCGATGAGTGCTCTG
TTTACTGTAGCCTCCAATAATTATGGGAAGATCAAGAGGTAGTGTTGATTGAGGTTGCCAGTAAATTGTA
TTCTGGGCCTTAGAGGACTCCCATATTTCCTGGAGGTTGATAGCCATTTCGGGCTGGCTTCTCCTTCCAG
GCCTCCTTCCTCCTTCCTCCTTCCTGTTGGAGTCAGGACATTTCTGTTATCTCTTGCCTAAAGACAGTGG
ATAAGAAGAAAGAGTGTATAATGTATAAGGCCTTGGCAGCTGGCTGTCAGGTTGCCCCCTGTTCCTTGAA
TTCATCCCCAAAGTAACTCTTATCTTTTATAGTAGTCCTCTCTTGGCAAGCTTTTAGGGCTGTGGTATTT
TCCTTCAAAAAGATCCCACCTACCTACCATCAAGCTTTTCTCAGAATTCATGGGTCACCAAAAGCCCCCA
TTTTACAGTATTATATATTTAATTGTAAAAACTCTTCCATCATTGACATGATTGTATATTTAGAAAACCC
CATTGTCTCAGCCCAAAATCTCTTTAAGCTGATAAGCAACTTCAGCAAAGTCTCAGGATACAAAATCAGT
GTGCAAAAATCACAAGCATTCCTATACACCAATAACAGACAAACAGAGAGCCAAATCATGAGTGAACTCC
CACTCACAATTGCTACAAAGAGAATAAAATACCTAGGAATACAACTTACAAAGGATGTGAAGGACCTCTT
CAAGGAGAACTACAAACCACTGCTCAAGGAAATAAAAGAAGACACAAACAAACGGAAGAACATTCCATGC
TCATGGATAGGAAGAATCAATATCATCAAAATGGCCATACTGCCCAAGGTAATTTATAGATTCAATGCCA
TCCCCATCAAGCTACCAATGACTTTCTTCACAGAATTGGAAAAAACTACTTTGAAGTTCATGTGGAACCA
AAAAAGAGCCCACATTGCCAAGACAATCCTAAGCAAAAAGAACAAAGCTGGAGGCATCATGCTACCTGAC
TTCAAACTGTACTACAAGTCTGTAGTAACCAAAACAGCATGGTACTGGTACCAAAACAGAGATATAGACC
AATGGAACAGAACAGAGGCCTCAGAAATAACACCACACATCTACAGCCATCCAATCTTTGACAAACCTGA
CAAAAGCAAGAAATGGGGAAAGGATTCCCTATTTAATAAATGGTGCTGGGAAAACTGGCTAGCCATATGT
AGAAAGCTGAAACTGGATCCCTTCCTTACACCTTATATAAAAATTAATTCAAGATGGATTAAAGACTTAA
ATGTTAGACCTAAAACCATAAAAATCCTAGAAGAAAATCTAGGCAATACCATTCAGGACCTAGTCATGGA
CAAGGACTTCATGACTAAAACACCAAAAGCAATGGCAGCAAAAGCCAAAATAGACAAATGGGATCTAATT
AAACTAAAGAGCTTCTGCACAGCAAAAGAAACTACCATCAGAGTGAACAGGCAACCTACAGAATGGGAGA
AAATTTTTGCAATCTAGCCATCTGACAAAGGGCTAATATCCAGAATCTACAAAGAACTTAAACAAATTTA
CAAGAAGAAAACAAACAACCCCATCAAAAATTGGGCAAAAAATATGAACAGACACTCTCAAGACATTTAT
GCAGCCAACAGACACATGGAAAAATGCTCATCATCACTGGTCATGAGAGAAATGCAAATCAAAACCACAA
TGAGATACCATCTCACACCAGTTAGAATGGCGATCATTAAAAAGTCAGGAAACAACAGGTGCTGGAGAGG
ATGTGGAGAAATAGGAATGCTTTTGCACTGTTGATGGGAGTGTAAACTAGTTCAACCATTGTGGAAGACA
GTGTGGCGATTCCTCAAGCATCTAGAACTAGAAATACCATTTGATCCAGCTATCCCATTACTGGGTATAT
ACCCAAAGGATTATAAATCATGCTTCTATAAAGATACATGCACACGTATGTTTATTGCGGCACTATTCAC
AATAGCAAAGACTTGGAATGAACCTAAATGTCCATCAATGATAGACTGGATTAAGAAAATGTGGCACAAA
TACACCATGGAATGCTATGCAGCCATAAAAAAGGATGAGTTCATGTCCTTTGCATGGACATGGATGTAGC
TGGAAACCATCATTCTGAGCAAACTCTCACAAGGACAGAAAACCAAACACCACATGTTCTCACTCACAGG
TAGGAATTGAACAATGAGAACACTTGGACACAGGGTGGGGAACATCACACCCTGGGGCTTGTTGTGGAGT
GGGGGGCAAGGGGAGGGATAGCATTAGGAGAAATACCCAATGTAAATGATGAGGTAATGGGTGCAGCAAA
CCAACATGGCACGTGTATACCTATGTAACAAACCTGCACGTTGTGCATGTGTACCCTAGAACTTGAAGTA
TAATTAAAAAAAAAAAGGTCAAAAAAAATTCTTCCATCATTTCTAGCAGTTGGGAGTATAGCATGAATGC
TAGGACATCCGTGTCCTTAGTTCATTGCTTGATGCAGTCTCTTAGGTTACATTTTCATCTTATTTTCTCT
TGACTAGATTAAGAGAGTCACCATTCTTCATAGGTCTTGCCTTTGAGTCCTCAGGATGTGTAGCAGTTCT
ACACCAGAGCTACTCAAAGTGTGGCCCTACCAATCAGCAGCATCAACATCCCTCGGGCAGAGACCAAGAA
ATATGACTTTTTAAAAACATCTGTCCAGACATACTCTCTGGAAGTTCAAGAAATGCTGCTCTACACTACT
TCTTTAGAAGCAGTTCCCATAACTGCATATCAATCCCTGGTAACAATCTGAATACTTTATGGTGACATGA
TAGAATTTTATTTACGGTTTTTATTTTTGTTGATGAATTCCTATAAATTGTTAATATTTTTGCTGTATTC
TTGATAATAGTAAATTCTCATAAAGATAATTTTTTGCCAAGCTACCCATAGTGATCTATTATGTGTGAAC
AGTTTGTCTTTTCTCCTCAAATATTCACCTTTTCTAAATTGTTCTTCCTGTTCTCTACCGCTTCTTAAGT
GGCAGATATCCTTTCAACATTTACCTTCCATAGTGCTTTATCATCGTTCTCTTACCACCAAGATGGTTTT
AGATCTAAAATTAATTAATATGCTTTTTGAGTCTATCATCTAAGTTGTTAACAAAAAGACACATTTAGCA
TGAGTTGTAAATATCTTAAATTTTTGGAATATATGAGCTTAGTGGGTGGGCTGGTAGAGGAAGAGGTATG
TGGGGGTGTGTGTGAAGGTGTATACTTTGCACATAGCCTAGCAGAATATATTGCATAGAGTTAGGGCGGG
ATGGATGTTTGTGCAATTGAAAGGTAGGCCGGAGAGTTGCCTGTGTGCATCACTCATGTAGCGTCTCAAA
TAGGCAGGCAGGTCTCCAGCCTGACTCATCAATCTTCCTCCCTAAAAGATCTCTAAGCGCAACTTTTCTC
TCATCTGAAAAGAGGAAATGAGAAGAGTCAGAGATTATTACTGGGGACTGAGTGGGTGGAAAGACTGTGG
TTCTGGGGACATTTATCAAGTATTGCAGTTCATGGCTGCAGATTATATCTTCTGTTTAGGCTGGAAATGA
TCCAGGATAATTTTCCTGAAAAATTTAAAAAATCTCATAAAGACAGGTAGTTCTTGGGTATACCAAAATT
TGGGCAGGCCAACTGATAACCTTCCCAAGAACTTCAAGGTGCACGCAGCCTTCATGGCTCCTCTTTTGGT
CTGAAGTTTGTCAGAGTGTGGAGATCCAAGTTCAAGTGGAGAGTTACGCAACACTTTCCAGATTGAATTA
CACTAAGCATTAGGTCTTGACTGGTTGCCAGAGCAGCACTTCCAAATAATTTCTGCACAAGAATTCAACT
GCAGAAGGCTAGACGGTAACCATGTACTGCTGGGAACATCCTTTCTGTGTGGTTGTATTGTAGCTTCCAA
ACCATCTGCCCCAAACCATAGAGCTACAGGATAAGCTGACGATGCTCAGAATGTTGCTCCTTCTAGATGG
TTTGTAGCAAAGAATAGGATAGAATTTAAACATTCTGTCACTGAATTGTTGACTTTACTTGGTGGCAAGT
GAAACTCTATTTAAAGCCCTATGACAACACCATTTATCAGGCCTCTAAAAATAATGCAAAAAGAAAGTAA
GCTAATTTTCCCACTTTTTTTGTTCCTCTGGCGATCTGTGCATCTGAAATTATGAGATCAATTTCTATAT
TTTCTGAGACTTTTTGAATCTTTGGAAATGAACTTTAGACACTGAAGTGCTAAAAAATTAGAAAAAATAA
ATGCATAGATGCCTTCTAGAAGGAAAAAAAAACACAGGAAAACAGCATAGCATTGTTTATATGGTGCTTA
AGTTTGGGAGCTCTTGGCTGCTTCGACATTTGAAGTCATACTTGTGAAAGGAGAGGAATTAGAAACTTAG
ATTCATAGGTGGATGGGACTCTCCTGACTTTAAGCATATACTGGAAAGTAGTTTGCCTGTTATGCTGTGT
TCTGATTTAATTGTTTCAATTTGTAAATCTTAAAAGTGGACAGAGAACTTCAGATATGTAGTAGAAAGTT
ATGTGGTATTATGATACCTATTTTAATTGTTATTATTAATGACAGCAAAAATTATGATGGCTGCAGCGTG
GTTGACAGCTGTTTTGCAAAGGTTATCATGGTGTCAGAGATGTTACCTGATTTCATTAAACCGGCTTCTA
CTTGAGATAGAAGAATACACTTGGCAGAATTTATAATTCACTGGAGAACTGTGGACTTGAAAAGTTTTGT
TCTCACACAAGCTTTTAAGAATCCAAGTTAAAATTTTATGAATGGTGATCACAAAAGCTTCTAAAAGGGC
TCAAAAAGCATGTTACGCATCAAATTGCTTTTTAAATTCAATCTTCTGATAGGTATAAATGAACTCAGTC
CAAGAGATTGAATTATCTTAAAAACTATATATAAGGGCTCAACACATTCAGAAAAGTACCTGGACAAATT
TATCTGGTTGAATTATAAAATTCTTTAAAGAAAAGATTCTAAACATCATTCAGTAGAGCAAATTAGGAAT
GTATCGGATGTGTTGGTTTACAGGGAGCACTCTGCTAGCACAGGCATCTCATATTCTTAGTAAATTCAGT
TAAGAATTTTACTGATTGTATCATAGCTTTGCTTTGACTAGGAATATCCTGAACTGCTAACTAAATTAAT
TCCAATTTTATCCTTGCTACTCCTGCCAGCTGGGATGCCAGCATGGACATCCTTCAGTGGACTTCGCTCT
GTGCCAGGGAATTTGTTCATATTACTTTTGAAAAACAAGCATTCTTAACAACAACAACAACACCAAAAAC
CAATTATAAACTAGAGAATAAACCTCTTCCAAAGTGGCACAATCTGGCACAAAGGCAAATTTTTAATCTA
TAAAAGTTGTTTCAGCCTTGGCACAGTGGCTCACTCCTGTAATCCCAGCAATTTGGGAGGCCAGGGTGGG
AGGATTGCTTGAACCCAGGAGTTTGAGACCAGCCTGGGCAACATGGAGAAACCCCATATCTACAAAAAAA
AAAAAAACCCAAAAAAAGCCAGATGTGGTGGCAAGCACCTGTAATCCCAGCTACTAGGGAGGCTGAGGTG
GGAGCATCGCTTGAGCCTGGGGAGGTTGAGGTTGCAGTGAGCCAAGACTGCACCACTGCACTCTAGCCTG
GGTGACAGAGCAAGACCCTGTCTCAAAAAGAAAGAAAGAGAAAAGAAGATTGTTTCAGCTGATACCATCC
AGCAGAGTCTTTTAATTGTGGTCCAGGAGACTTTAAAAACCTCTGTGAACTGCTTGTCTTCTGTCCAAGG
GTGAGAAAATGAAAAGCACAAATAATTCTATAGAAATACCCTGAAAAGAGACCAGTAGAATCAAATAATG
GGAAGAGTGCAAAAAAAGATGTAAATAAAGGGGAAAAAATACTCCAGAGGGAAATAAATACTAAGCAATT
TAAGACTGATTAAGGATGATTAGTTTTTAATGAGCTACTTTTGCTGTTTTGGAAATAAGAACCTGAGGCA
GGAAGTAACTCCTTTATTACGAATATTTTCTTTTTCCTCCCTCCCTCCCTCCCTCCCTCCCTCCCTTCCT
CCCTTCCTTCCTCCCCTCCCCTCCCCTCCCCTCCTCTCCCCTTCCGTCCCCTTCCTTCCCCTCCCCTTTC
CTCCCCTCCCCTCCCCTCCCCTCCCCTCTCTCTTTTCTCTCGCTCTCTCTTTCCTTTCTTTTTTTGACAG
GGTCTCACTTTGTTGCCCAGGCCAGAGTGCAGTGGCATGATCTCAGCTCACCCCAACCTCCACCTCCTGG
ACTCAAGCCATCCTCCCACCTCAGCCTCCCGAGTGGCTGGGACTACAGGCGTGTGCCACCACACCCAGCT
AATTTTTGTATTTTTTTGTAGAGGCAGGGTTTCACCATGTTGCCCAGGCTTATCTCAAACTCCTGGGCTG
AAGCAGTCCACCCACCTTAACCTCCCAAAGTTCTGGGATTGCAGACATAAGCTACCACACCTAGCATGAA
TATTTTCATTTTCAGTATTTTACTAGTTAACATTGTATTTTAGTTTTTTAAAAGTTTTATTATAAAACAC
TTATGTCATGGAAAAACTGTGTGCCTGTTGCCCAGGTTTTATAGTGCTAACATTTTATTTATATTTGATA
TTTTTAAGAAAAAGTTTCAATTAGAGCTGAAGCATCCTTCTCCTATTCCATTTCTTTATTCTCCCCGCTG
GAGCAAGCACGCCCAAGAAGTGGGGTGTAGATTCTTCCTGCCATGTTTTAACACTTTTACTTCACATTTC
GCTGTGCTTTAAAACTTTACATAAGGATGGTGTATTGTGGATATCTTTATGCAACTTGCTTTCTTCATTC
ATTCAACTTTTGAGATTTATGTATGCAGATAATGTAGGTACAGTTTATTCATTTAAACTGCCATATAGCA
TTCCATTCTGTAAATAAATAATCATTTATTCATTTCTGTACAAATTCACAGCAAGATTGAATCTACCTTT
TACAAACAGTCCTGTTTGTCTCCCTAACCTGTAAGAAGAAATGGTAGCACGTTGCTACCAGTTGTTCTCC
AAAGTGTTTGTGCTTATTTATTTATGTTCCTATCAGAAACATCCTGGAGTTCTCTTTTCTCTGTAATCTG
ATGGATTTCCCACTACAATTTTTTTTTTTTTTTTTTTTTTTTTTGAGACGGAGTCTTGCTCTGTCACCCT
GGCTGGAATGCAGTGGCGTGATGTCAGCTCACTGCAATCTCCACCTCCTGGATTCAAGCAATTCTCCTGT
CTCAGCCTCCTGAGTAGCTGGGACTACAGGTGCCCACCACCATGCCTGGCTAATTTTTGTATTTTTAGTA
GAGATGGGGTTTCACCATATTGATCAGGCTGGTCTCGTACTCCTGACCTCAGGTGATCTGCCCTCCTCGG
CCTCCCAAAGTGCTGGGATTACAGGCATGAGCCACCACACCCAGCCCTCCCACTACAAATTTGATGTTAT
AGTTTGTATTGAACAACAAACAACACATTGAAGTACTAAAGAATTAATTAGGGTTGAAAGTAATTTCTGG
ACATCAGAAAATGGGGATAGGAATGCCCTTTCAAAAGCTACTATTTTGATTAACTTTGAATAACTATTTG
ACCATTTCAAGTAGTTACATCAAAGAAATAAACAGACAACCAACAGAGTGGGAGAAAATATTGCAAACTA
TGCATCTGACAAAGGACTAGTATTCAGAATCTACAAGGAACTCAAATCAAGAAAAAAGCAATCCCATCAA
ACAGTGGGCAAAGGATATGATAGATATTTCTCAAAAGAAGATGTACAAATGGCCAAGCATATGAAAAAAT
GCTCAACATCACGAATCATCAGGGAAATGCAAATTAAAACCACAATGAGATACCACCTTACTCCTGCAAG
AATGGCCTTTTAGATGTTGGCATGGATGTGGGGAAAAGAGAACACTTATACCTGCTGGTGGGAATATAAA
TTAGTGCAACCTCTATGGAAAACAGTATGGAGATTCCTCAAAAAGCTAAAAGTAGATCTATTTGATCCAG
CAATTACACTACTGGCTATCTACCCAAAGGAAAAGATGTCTTTATATAAAAAAAAAACCACTTGCATATG
TATTTTTATAGCATCACAGTTAACAATTGCAAAGATGTGGAACCAACATAAGTGCCCATCCACCAATCAG
TGGATAAAGAAAATATGGTATATATACACTACTCAGCCATTAAAAGGAACAAAATAATATTTGCAGCAAC
TTGAATGGAGCTGGAGGCCATTATTTTAAGTGAAGTAACACAGGAGTGGAAAATCACAAACTGTTATGTT
CTCACTTATTAGTGGGAGCTAAGCTATGAGTACACAGAGACATACAACAAGCTTGTCGAACCTGTGGCCC
ATGGGCTACATGCAGCCCAGGACAGCTTTGAATGCTGCCCAACACAAATCTGTAAACTTGCTTAAAACAT
AGTTTTTTTGCGATTTTTTTTAAGCTCATCAACTATCGTTGGTATTAGTGTATTTAGTGTGTAACCCAAG
ACGATTCTTCTTTTTCCAAAAAGAAGGAAGCCAAAAAGATTGGACATCGCTGGCATACAGAGTGACATAA
TGGACTTCAGAGACTCAGAAGGGGGAGGAGAGTGGGATGGGGGGTAGTGAAAAAAACTACACATTAGGTG
CAGTCTACTCGGGTGATGGATGCATTAAAATCTCAGAATTTACCACTATACAATTCATCCATGTAACAAA
AAACTACTTTTACCCACAAGCTATTAAAATAAAAATTTTAAAGTAAATAGTCTAAACAAAAAATATAGTT
GCATTTATTAATTTCATAAAAATGAAAACTAAATTTTTACAAAAAGAATATGCAGGCCCCTACAGTGATC
TACAGAAGCCTTTCCGCCATCCTCCCCGGTTCACTGGGTGTGGCCACGTTGGTCCCCTTGCTATTTTCAG
ATGCTGAGGGCCTTTGCTTTTGCTTTTCATCTGTCTGCAATGTTTTCTTCCCAGATATATACGTGCTTGT
TCATGTACTTCCTTCAGAGCTCTGCTCACATATCACCTTCTTGGTAAATTTCTGTCACTTCTGAGTATTC
TTTACTTTTCAGCATGACACTCAGTATCATCTGGCATTATATTGTTCCTCTCCCTGATGAGACATTGTCA
TCTTTGCATCTGTGTCATGTACACCTCAAACAGAGCCTTGACTACAGTAGGGGATCAATAAATAGGTATT
AAATGAACAAATAGGACAAAGATACACAAATATATAAACAAATTCTCTGAAGAGGAAATACAAGTGGGTA
ACAATTACTGTCTCACAAGTTATCAGGAAAATGCAAATTAAGGCAAAGAAATAGCAATTTTTGTCCGTTA
AATTTGCAAGAAATAGAAACAAATAATGATATAATTGGAAAGGGTGGAAAAGTAAATTGGTGTGGTCTTT
TGCAAGGCAGTTTTTTTTTGTTTTGTTTTTTGTTTTTTGTTTTTTTTTTTTTGAGACAGGTCTCGCTCTT
GTCTCCCAGGCTGGAGTGCAGTGGTATGAACACAGCTCACTGCAGCCTTGACCTCTTGGGCTCAAGCAAC
CCTCCCACCTCCGCCCCCCCGAGTAGCTGCAACGCAGGCGTAAGCCACCACACCTGGCTAATTTTTGTAT
TTTTTGTAGAGTTGGGGTTTCTCCATGTTACCTGTGCTGGTCTTAAACTTCTGGGATCAAGCGATCTGCC
TGCCTCAGCCTTTCAAAGTGCTGGGTCTCCATGTGTGAGCCACCACCCCTGGCCTTGGAAGGCAATTCAG
TATGCAAATACTCACACATGGGCACAGACAGGCATGGGCAAGGGTAGCACCACAGCCCTGTCTGAATAGT
GAAAAACCAGAAACAGCCTCAGTGCCTACTGATAGTGGCATTGTTAAATTACAGAACATGTTTGTTTTAA
TAATAGGCAGCAAGTTTTTAAGAATGAGGAACAGCTACATGCTCTATACTGTGGTTTCATTTGTGTTTCA
CAAAACACAAGACTAAATTTCTATATTTACAATTACATATGAAAATACCTAGAATAGCCTCCACCCAGAA
CTTTCATTTTATTTATCATTCACTGGAGACGGATTTGTTGAGCACGTGCTGTGTGTGAGGAGTTATCCCT
AGGTGCTAGGAATAGAGCGGTGGGCAAAGTAGGAGAAGCTGCCTGCCATTGTAGAGCTTCTGCCCTCAGA
GGAGGAAGCGAACCATGAGTACTATAAGGGAATACTGTGTAGGTCAGGTGGTGAAAGGTACCAGGAAGAG
AAGTAAAGCACGGGTAAGAGGGCACTTGAAGCTCGAGTGTCCAGTGGCGCTTTCTGCAGTCCCAGGCACA
TCCTGTAAATGTGCACTGTTCGGTACTGTAGCCGCCAGCCACCTATAGCTGTTGAGCACTTGAACTGTGA
CTAGTGCAGTTGAGGAGCTGGATTTTTAATTTTATTTCATTTTAATTCATTTGCATTTAAATTGCCACAC
ATGGTAGACAGTATAGTGACATGGGATGATCAGAGAAGACCTCACTTAACAAAGGGACATTTGAGCTGCA
ACCAGGGAGCTAATCATGTATTTCAGGCAGAGTGAACAGCAGGACTTTATTTGGGGCAGTCAGAGGCAGA
AAAGACAAAGGTAAGAGTGGAAGGAGATTAAGTCAGTGAAACAGATGGGGACCCACCATGGAAGGACTTG
CTGGCTAGTGAAAGACATTGCTTATTCTCTACATGAGGTGAGAAGCCATTGCAAAGCTTTGAGCAGACAA
GTGTTGTGATCGGAATTTTTAAAGGGGTGCTGGAACTGTTCTGATGTTAAACTATAGGAGTGAGTGCAGA
AACAAGAAATCCAGAGGCTACTCTGTCACCCAGGCTAGAGGTGGTGGTGAGTTACTCTCCACTTAGGAGG
ACAGTGATGGAGGTGTTCAGAAGAGGTCAGATGTGTTGCTAGTATTCACTGATGGATCAGATGAGGGGCC
TGAGAGAAAGGGAGGAATCAGGATTTGGGCCTGAGCAAACAAGATAGCACTGGGGAAAGGGAACACTGGG
AAGGGAATTCAGGAGTTGTGTTTTGGACATATTAAGTGCCTGTGAATATAAAGAACAAAGTCCTGACCAT
GAATGACACTGAAACAGCAATGGTAGCTAAAAGATTTCTTCCTAAAGAATTGGATAAAAGAGAAGTGTTC
ATCGATGGTGTTAGAGTCATTCTCTTTATTTAGAGGCAAAACAGGTGATCTGTCTTTGACCTTTTTAGCA
TGAAGGTTTTTATATGTCCTTAATTATGCATGGCTCTTAGATTTGAGTGATTATTTCTTATTTCTTCCAT
AGAATGGCTCCCGAGCGAGTGGCGTCCCTGTCACGAGTTTGTGTCCCACTTATTACCCTGACAGATGTTG
ACCCCCTGGTGGAGGCTCTCCTCATCTGTCATGGACGTGAACCTCAGGAAATCCTCCAGCCAGAGTTCTT
TGAGGCTGTAAACGAGGCCATTTTGCTGTAAGTGGCAAATGTTTCCTGTCATCCTGCGTCGTTTTTCCTT
TTCTTAGAAGGCTGTGGTGTGTTGGAAAGACTGTACGACAGCAGTGTCCAAACGTTTTTGTCTCAGTACC
CTTTATACTCTTAAAAATTACTGAGTATCCCGGCCAGGTGTGGTGGCTCACACCTGTAATCCCAGCACTT
TGGGAGGCCAAGGCGGGTGGATCACCTGAGGTCAGGAGTTTGAGACCAGCCTGGCCAACATGGTGAAACC
CTGTCTCTACTAAAAATGCAAAAATTAGTCAAGCATGGTGGTGTGTGCCTCTAATCCCAGCTACTAGGGG
GGCTGAGTCAGGAGGATCGCTTCCACCTGGGAGGCGGAGATTGCAGTGAGCCGAGATTGTGCCACTGCAC
TCCAGCCTGGGCAACAGAGCGAGACTCCATCTCAAAAAAAAAAAAAATTACTGAGGATCCCAAAACATTT
TATTTTTTTATTTTTAACTTTTATTTTAGGTTTGGGGGTACATGTGAAGGTTTGTTACATAGGTAAACAC
ATGTCATAGGGTTTTGTTGTACATATTATTTCATCACCCAGGTATTAAGCTCAGTCCCCAGTAGTTACCT
TTTCTGCTCCTCTCCCTCCTACCGTTGCTCAGGTAGGCCCCAGCGACTATTGTTTCCTTCTTTGTGTTCA
TACATTCCCAAGAGTTTTAAAAATCTGTTAAACCCTTATTAATATTTATCATATTTGAAACTAAATTTTT
AAAAATGTGTTTGTTACTTAAAAAATAAAAATAATTTTATTACACGTTAACACAAATAGCTTATTTTTAT
GAAAAGCAATGCTCTTTTCCAAGGCAAAAAAAAAAATGTTTGACAAAAGCAGCATTGTTTTTACATTTTT
GCAAGTCTCATTTTCTACCCTAAGACTGCTGAATTCTCATATCTGCTTCTGGTGGGACGTGTTGTTTTGG
TTGAAGTGTATGAAGAAAATCCAGCCCCACATAGATAGTAATTGGGAAAGGCGGGAATATTCTAGTAGCC
ATTGCAGATCATTGTGGATAATTCTGCTTTGATAGCTCACCAAAATTTGAAAATGGTCATTTTTTAAAGG
GTAGTTGCAATGTGGAATCTGAAATTATGTCAGTTAACTTTCCATACTCTGTTACATTAGAAACCCATTG
GTCTGTCTTGCACTTTCAATGGATCTTTTTTGCCAACACACATAATTTTGTAGCATTATGCATGGTTCAT
TTGGAAAATCTTCATTCATTGAGTTATGTATATCTTCCAAACATTGACATATTTCATATGCAGTATCAAA
ACCTATAGTCATTACTGCCACCATGGAGCTCATCAGAAATGACTTTAAGTATTGGGAAGCTGTCAAGCTT
ATGTTGGTGGACATAGATTTTCCAAGATTCTAATTTCTGCTTAAAAGCTAAAATTTATCATGGGCTACAA
ACACTGTCAGTCATTTTCCTTAGGGGACTGAGTCACTTTGCTAATTATTGAGAAAGTATGTGCCTAATCC
TAAAATGTGACTGACCACTGCCTGTCTGCTAGTCATTCTTTCAGGTCAATTTCCATGACAAAAAGTGGTC
AAGTGAGCCCACTGCTGAAAGAGTTGTGCAGGTGTACAAGTGGAGTGCTTTTCTCCAGGCAACCGTCTTT
TTTTGTTATGAAGCAGAAGTGCTTTATGCACACTTTCCTTTTCACCGTACAGAATTCTTTTTTAAAAGTA
TCCTTGAGGGCAACAGTTTAATAAAGTCAGTATGTTAAACTGCTTCATCAGACATTCATATGTGAAAATG
CAGTGTTTCGACTGTGTGTGTGTGGTGGTGAAGAGTAGCGTGAGGGCTGTCTCGGCTCTGATTCCCACTG
AGACACAGCTGTGTTACCCACTGTTGCTTTTGCGCTATCAGTGCAAATGTCAACACGGTTATTCCAGGAA
AAACCAGGAGATTCAAAATAGTTATTCAGTACTTTAAGTATTTCCCTACCACTTGGGTGTGTGGCCAACT
TTCATATAAAAGAGCTTCTGTGCTTAAGTTGGTGCTGATACCAGACAAGTACAAGCAATACAGTAAGTCC
AGAGATTCTATTGTAAGGCAGAAGTACAGTTCTCTTGGTGACATATTAATTTATCACATCTGCATTTAAA
TTTTTTTTCTTCAGACTCTGAATGATTGGTCTCAAACTGATGTGGCAGTTAACTTGCACCATAACAGTAT
TCAAAATATTGTTGCATAAGACACAATAAGGTAAATCATTAACATCTGTAAAGGCAAGGGAAAAATAACT
TTCCTCACATTTTTTGTCTTTATTTACAGTTTAGCCCACTTTCTCTGTAGCAGATGTCTCCATCTCATGA
GTCAGAGAATTCTCTGATGTGTCAGTTTCATCTTTTCTTTTCTTTTCTTTTTTGAGACAGAGTTTCACTC
TTGTTGCCCAGGCTGGAGTACAGTGGTGCAATCTCGGCTCACCGCAACCTCTGCCTTCCGGGTTCAAGTG
ATTCTCCTGCCTCAGCCTCCCGAGTAGCTGGGATTACAGGCATGCACCATCACGCCCAGCTAATTTTGTG
TTTTTAGTAGAGACAGGGTTTTCCATGTTGGTCAGGCTGGTCTCGAAATCCCGACCTCAGGTGATCCACC
CACCTCGGCCTCCCAAAGTGCTGGGATTACAGGCGTGAGCCACTGCGCCAGCCAGTTTCATCTTTTCTTG
ATGAAACATCTGTAGGATGAGAAATTGAATCTTTTAATCCTCATTTTTAAAGCCAGTGATCCATTCTTAA
ATGCAAGAAAATACATCATAAATAAATGTTACTTTTCAAATAGAATATTGTAAAATTCCAAAATAATAAT
CTAAAATATATATTAAAATATATAAGTAAAATATACACTGTATATATACATACAAATATATATCTAATAA
AATTATCTAAAATAACTTAGATAAAATTTAAAAATTAGAAAAGTTTTCAAAATTAAAAAATATATATTAC
TACAAAACCCAACAAAGTGTACTTATTTCTTCTTATGTTTCCACGTAGGCCCAAGAAAAATGTAAGAATT
TTTTAAATAATGATTTTTTCTTTTAACGCATCTCAGTTCTTCCCTCCCTCTCTGGAAACAGTTCTTTGAA
CCCCACTTTGAAAATTGTTGCATGGCCTCTGACTACTGCTGTAGTCATGACTCCAAGTTACGAGTGGCTC
TGTCTGCAGTGTTTGAAAACACTGTGAATCCACTGTGTGGTATAATTTGCAGAGCCCTCTCCGTCTCAGA
TAATGAGATCCAAAGCATTCACATTTCCGAATTCAAGGTATACATGCAATGTGATTTTAAAATGATACTT
ACTGCTTATAAACTTGACATTTTGTTAAGAATACTGTGGTTTTTAAATCATTTTATTAATACTATTTACG
TTTTCAATTTTTTTTTTTTTTTTTTTTTTTTTGAGATGGGGTCTCACTCTGTCACATAGGCTGGAGTGCA
GTGGTGCAGTCTTGGCTCACTGTAGTCTCAAACTCCCAGGCTCAGGCGATCCTCCCACCTCAGCCTCCTG
AGTAGCTGGGACCACAGGTGTGTGCTACCATGCCTGGCTAATTTTTTGTATTTTCGGTAGAGACGGCATT
TCACCATGTTGCCTAGGCTGGTCTTGAACTCCTGGGCTCAAGGAGTCTGCCTGTCTTGGCCTCCCAAAAT
GTTGGGATTATAGGCATGAGCCACTGTGACAGGCCCAATTCTTTATTTAAATACTTTCAGTGAGAAATTG
GAGAATTGTTACATATGTAGACCCTTCCTTGCTATACAATGCATTTTAAAAAAACAAAACTACAATACCA
TTATTACATCTTAAAATTGACATTAGTTCCTTATCATCATCACATATCTAGTCAGGAATTAAATTATCCT
GATTGTCTTAGAATTTTTCTTCCAGTTGGTTTGTTAGAATCAGAGTTCAAATAAAAAAAAATAAAGAATC
AGAGTTCAGATCTACACATTGCCTTTGGTTTATGGTTCTTTTAAGTCTCTTTCAATCCCTCTTTGTTTTT
TGTTTTGAAATTATTTGTTGAAATAATACGGTTGTTTTCCCCGTTCTCCGGTGGTAAAGCATGTCCGCCC
TCTTGAGTGTGATAAAGGGTGCGTGCCTCTGGAGCACAGTCCTGTGGGCTTAGGTGGTTCCCTTGTCACA
TTCTCAGTTCTCACATAGTCAGGCCTGTTTTGTTACCTTCTGTTCTGGTTCTCTGGTCTCTTGCATGTGC
GTGCGGCAGTATCACTGCTTTAGTGCTGGCATCAACTGTGTTCAATACAAAATAAGGATCATTCCCTTTG
ATTTCTCTTCTTTACTAGGGTTGTTATTAGTTTGTTTTGCTATCATTGCTTGTCTTATATTCCACATAAA
CTTTGAAACCAATTTTTCTAGTTGCAGGGAAGACACCCAGTGTTGTTGGGGGAAGACTCCTGTAAGTATA
TAGATTATCGAGAGAGCTGGCGTTTTCATGATGTTAAGGCTGTCTGTCCGCCTGTTCAAGAACACATACG
TTTTACCATTTGTTAAGCTTTTGTGATCTGAAGGGAGTGTTGTATATATTTTTCACACATTGGATTTACA
CATTTCTTGTTCCTAATGTTTTGTTGTATTTGTGTTTTCAATGTTTTCACTGTCATAAATGGATCTTCTC
CTCCATCTTCAAATAGATTTTCATTGTGTTGTTTGTTGCGTTTGGAAAACAAAACAGGCTCAAAACCACC
GTTGTTCTTACAGTTCAGAGTATTTCCATTTTTTAAATCTATAACTCTTAAAAATTAGGATACATATTTT
AATTTCAGTTTATATGAAAGGTTATAATCCAGGATTTTGTGAAAGTGAGATTTAAAAAGAGAATTAGAAG
ATTAAGTCACTTGATTCGAAAAATGGTCAAAATGCAGGTTTCATGGCTAAAAAAATAAAATGGCCTTTTT
TCTACCTACTCTTAAACCTTCTGTCAAAATTCACCCAGTAAGTGGAGTGAGCTTTCTGAGGAGTCCGAGC
GTTATGTTTGGTCCTTATGTGAGATACTGAGTAAAGGGCGTTGTGAACGTGCGGCGCGGCGCTCGATTCT
TTCCTTACAGTGCCCAACACCCGCAATGGAGCACACGCTTCACGGCCCCACCACTGATCACACCAACTGG
CATTTTTACTCTCCACGGCGATTTTTCAAAATACTTACAAAGAATATTTAACTAAAATGCTCTTGCCTTT
CACTGTGGCAGCTCCCTTCTTGATTCATCTAGGAATATATTTCACAGGAGAATATGGTCTCAGCCACCCC
CGTGTCTAGCCTTACTCTTTCAGCAAGAGCTGAATTAAAGGGAATCTGGAAGTGAATACTCCTGGTCTTC
TGTGAAACCCCAAAAGCTGTTTGAGAAATCACTGGCCAGGCGACGTGCCCAGAGAACAGCTACTCCTGCT
CTAGCACAGTGCTCAGAGGGACGCTGAGGGCAGCGGCTCCCCAAGCAGGCCCACACTCCCTTGCTTGTAA
TTTTGCTTGTGATAGCATGTTCTCCCTCAACAACTCATAAAATTTATTTGGTTTGGAAAGTTATGTCTGC
TAATCCATAAGGGACAGACTGTCCAGAAGTGCTGGGTGGTCTTCAGTGTGTCCAAATCCTGTGGCTTCAG
CTCAGCAAGTTCCCCAAGCAGTGGCGTAAGAGCTGGAGCCTCTCCATGGACACTTTCCCGTAGGCGGAAC
ATCCCAGCCACATGTTGACACAGCGAGGCTGATCCAATCAATCAGTGCAAATTACCAGCCGCGCCCAGCC
CCCCCTCCCCAGATGTGCTGCGTGGTCATTTGGAAACCACATGCTTTCTTTGACCAATGCACTCTAACCC
GTGTTTAGCCTTCCAACTATTTGCTTACCACAGTACACCAGTGGAATTTCATCTGTTTGACAATTGGCTG
GTGTCTTTGGTCTTGGCTTCCAAATGCAGTCATATATTTTCAGCAGTGGTCTGCCTGTCTTTTAAACAGC
AGATGCTTGCCCCTCTTTATGGTTTAGATAACCTAGAGGAACTTTTAAATTTCTTGGCCTAAATGTCAAC
TTTTCATGTCTGTCCCTGGGGCTGCCGTCATAGGCACCTGCAGGCTAAAGACGGCATAAAACCTTCCTGG
GCGGGCTGCTGAGGTCCTTTTGTAAGTCTGGAGTATAAGTAATACAGCATGACATGTCCTCTGAGGTCAT
CCGAATCCACTGCTTCAAAAATACTGAGAAGCTTGAAGACTGGATGGAGAATTTGTTCTATATGCCTGAA
TGAATGAGACACTGAACAAGTGAAAAAAAGAATGAAGGCATGACGGTGATCTCGTGATATAATAAGAAAT
GTGCATTTGGTCTTCATCCAGGCTCCTAAAACCCTTGAAATTTCCTGAGTGATGAGGTGAGAAGAGCATC
TTTTATTATTCATAAGTCCCTTTTCACCACACCTGAGTGTAGGCTAATGAAGTGACTCCTGGTGGGCCCC
CAGATAGCCTCGGGATGGGGGCTGGTAGCCAAAGGAACCAACCACACCTGACCCCCAGAGTTCGACCTGA
TCACTACTGGCCAGCGATTTAATCAGTCGTGCTTACATTGTGAGACCTCCATAAAAAACCTAAACTGTGG
GGTTCAAATAGCTTCCAGATTGGTGGTCACATCCAGGTGCTGGGAAGGTGACGTGCCTGGAGAGGGCCTG
GAAGCTCAGAACTTTCCCCCAGGCCTGGCCCCATGCATCTCTTCCATGTGGCTGTTCCTGAATTGTATCT
TTCAGAATAAATTGGTAATGGCAAGTAAAGTGTCTTCCTGAGTTCTGTGAGCCATTCTAGCAAATTATCA
AACCTGAAGAGAGGATCATTGGAACCTCTGATTTTATAGCTAGTTGGTCAGAAGAATGAGAGGCCTAGAC
CTGTGATTGGCATCTGAAGTGGGGGCAGTCTTGTGGGACTGAGCCCTTAACCTGTGGGGTGTGTGCTAAT
GCCAGATGGTGTCAGAATTGAATCACTGGACACCCAGTTGGTGTCTAGAGAGTTGGGAGCATTGGTTTGT
GTGGAGGAAGAAAATCCACACAGTGAGTGTGAGAAGTGCTCTGTGAGTAAAAACAGCTTCTAAGTAGTGG
GAGTGACTGACATACGAGTCTCTGACCACTCTAACACTCGCAGCTCGGGGTGGCTCTGAGTGTTAAATAG
GGTGGCCATATGATTTAAAATCTAGACCATGCCACTTCTGAGAGTTAAAAGAGATCCCTTTTATAAGTAT
AGTGGGATAACAGAAATAAACTTACAAACTAGGACAAATACTTACACTAAGATTTAAATGGGTTGAATGA
CTGTAATATTCTTAGCACAGTGCCTCGTATCCACAGTAAATACCCCGTAACCATTAGCGTCTACAGCCAC
TCCACCCTAAATGTGCCCAGTCTTGCCTGATCTCAGAAGCTGAGGGGGGTTGGGCCTGGGGATTAGCGGG
TGCTGTAGGCTTTAAAAAATAAAAATAAAAAGACAGGCACAGTGGCACACACCTGTAATCCCAGCTACTC
AGGAGGCCAAAGCAGGAGAATCACTTGAGGCCAGGAGTTCAAGTCCAGCCTGGGCAATGTAGCAAGACCC
TGGCTCTAAAAAATAAAAACTGTTGGTGGCCGGGCACGGTGGCTCACACCTGTAATCCCAGCACTTTGGA
AGGCTGAGGCAGGTGGATCACCTGAGGTCAGGAATTTAAGACCAGCCTGGCCAACGTGGTAAAATCCCAT
CTCTACTAAAAATACAAAAATTAGCTGGGTGTGATGGCAGGTGCCTGTAGTCCCAGCTACTCAGGAGGCT
GAGGCAGGAGAATTGCTTGAACCCGGGAGGCAGAGGTTGTAGTGAGCCGAGATCGTGCCATTGCACTCCA
GCCTGGGCGACAAGAGCAAAACTCTGCCTCAAAAAAAAAAAAAAAAAAAAAAAACCCCACAAACTGTTAA
CATATTACTATTACCATTATTGACTCTAGTAATTGTATTATTTTTGACAATGTGTAGTATTAGGTTTTTG
CAAACTCATATTTTTCCCCCTAACAATCACTGAACTAAAACCAGATGTAAGCTTAATTCCCATTGCTGAA
AGAGCTAATATGGCTTCCAACAATGCTTAATAAGAAAACAATTCATTATCTATCAGAAGAACCTAATTTA
TGCTTCTTGACTAACTTATGTATTAGTAGTTTACTCGTCTGTTATGCTCTTGCATATCAAGTTACCCCAA
AAAAGGAGCCTAACTAGAATTACTCTTGAGGAGCCACAGATTCTTTTGAATACCCTCAGCATCGCTGTAG
CCATTTTACAAATCAGGAAAATGAGACCTCTGGAAATGAAAACGTAAATTAGAAGAAAGGGCCGACCTAC
AATCAAGATGTTGGGTTTATGTTTTTTTCAACTTAGATAAGGAAAATCCAGATTTAAATGTTGTCAAATC
ATATAGGTGAGTGTTTAAACAAAGAAAAGTCAGCATAATACCAAGGACAGAAAGGTGGCAAAGAATTAGC
AGTAACTAAAACTCCCATTTCTATTAATATTGTTTATCCATGATGGAAACTGAGAGACTTCCTCACATCC
TTTTTTTTTTTTTTTTTTTTTTTTTTTTGAGACAGGGTCTCACTCTGTCACCCAGGCTAGAGTGCAGTGG
TACAGTCATGTCTCACTGTAGCCTCGACCTCCCAGGCTCAGGTGATTCTTTGACCTCAGCCTCCCGAGTG
GCTGGGACTACAGGCATGTGCCACCATGCCTTGCTAATTATTTTGTATTTCTGGTAGAGACTGGGTTTTG
CCATATTGCCCAGGCTGGTCTTGAACTCCTGGACTCAAGCAAACCACCCTCCTTGACCTCCCAAAGTGCT
GAGACTACAGGCCTGAGACACTGTGCCCAGCCACATCCTGTTTTTTTAACCTTAATAAGATATGGTCACA
AATTGAGTCACAGAAAACTTTACTTTTCTAATAAAAAATCTGCTTATATTGACGTGTAATTGCCAGACAT
TCTGGTGTGTTTGGGTTTGCACATTTCTTCTGCTTGTTGGTGGCTGCCTTTTGAGAAGTGAGCTGCTCTG
TACATGCAGATGATAATGATGGCAACTTTTGTCTGAATTATGATGCATTGCTTAGATCAGGCTGTCACCT
AAATACAACTTTAGGATCAAACAGTGTTCTTTTCAGTTCCTTGGAAGTAAGCAATTCTCCATAACAGTTA
AATCCAAGTTTTGTGAGTTTGTCTGGCAACCCAAACAGCCTTGATCTCTTTCTGGTTTGAAAAGGTGGAC
TGTTGCCAGTGCACCTTTGTCTTGGCTGAGGAAGCCCTGGCCTCCCCTCTACTGCTCAGACAGCTTTAGA
ACACAACCTCTCAGATGGGCCTCCCTCATCAAGAACCTTTCTTGTGAAGAATTTAATTCTGCATTCTGGT
TTTGTTTTGTTTTGTTTTGTTTTTTTGTAGCTATAAAAATATTTGGAGCCAAAATTATTTAGTAAGCAGT
TGGACCAATTTCTTTACCTGCTACCTCTTTTTGTAAAGGATGAGAAAACACAACTGCCTCACTAAACTGA
GTATTTGGGGGAAACAAAGTAGAGTTTTCTAAATCTCAAGGAGGTGTCTTTTGGAAGAGTCTGTTGGACA
GGGGAGAGCGTCCTGTCCAGGCTTCCCTGGCTCATGGCAGACGCTGTCACTGTAAATCAGATTTCTAGGT
GGAGCCCTGACTGGAAGCCTACTGCTCATTATTTTAAAGGAGAAAATAGTAATGTATTACACATAAATTC
TAAATGTCCACCCCATTTCCTAAAACATAACTATGATACAGAAAACTCTCCCAATTATTAGGTAGGGATT
TGGGGGAGTCAAGTTTTTATCTTACTCTGTTTTCTTGAATTCATGATTTTGGTCAATTCCAGGCAGGATG
GATGGAACCATTATAGAGAAGAAAAAGGACAACCTTAGCGTTTCTGTCACTAACGTATTGAACAGAATGA
AATTTTTTAGTTTCCCTAAAAACATTTTTTTCTTTTTTACTTTATTGTCCCTTTAAATTTTCCACAGAAT
GAATGAATATATATATTTATATATATATATATATAAATATATATATATTCATTTTGTCAGGTCAAACAAT
ACAGCACAAGGCATATACAGAAAATGTTCATTACCTCCTCCTCCTCATCCTGCAGTAATTCTGGTAGCCA
TACCTTATGCTTGTTAAACATAAAGTCCATATAAAGCCATTGTCTGCTCATTGTCAAAACAAAAGCAGCA
TTACACCATAGCCACATCACTCCAGAGCCTGCTTTTTAACCAGCGTGTCTTAAATACCCCTCGCAGGACA
CAGATCCAGCTCATTTGAAATAGGGGCATACTCCGTGGCACGAGGCCCACACTCTGTTCCGCCATCCCCG
TGGGTGCACACTGCAGTGTTTATAGTTTTGTCCTTATCATATATAAGGATTGCAGTAAATATCCTCAAAC
TGATGTTTCTATTTCTGCCAGGCAAATTGACAGCAGTACGATGATTAACACGGGGCTGTGTGTCTTGCAT
TTTCTTCAGTGGCACCACATGACTGGCCATGTGAAGCCAGTGCCTGCCTGCCTGTGAGGAGACGCACCCT
CTGCCTACCCCAGCACTGGGTGTTGTCACTCTCCTCAGCGCCTACACGGAGAGGGTGAAACTGAGACCAC
GTCGGTGCGTTTCCCTTCCTTTACTGTTAATTAGATGGGAGAGCTGCACATTTTTATCGGCCATTTGCAG
TTCTTTTTCTGTGATTTGCCTTTTTATTTAAAGCCAGCCTGGTTCAGACCACCAGGTAGAACACAAATAT
GTTGGCTCGGCCAAATTCTGTTAAGCAAGCATATCAAGATGATACTGCTTCGGCGATTCCTTCATTCAGG
GGTGATTACAAAGAGTTAAGTCACTGAGGTTTTAGAGCAAAATCTATCCAAATATTAAACACAGGTCCAT
AAACATAATCACTCACAGCAATTAGTAGGGTGTGCTCTGAATCAAGGCTCTAATAAAAAGACAGCTATTA
ACACACATCGTTTTCAAACTAGAATGGGCGCATCACGCACTTTACTGACTGGAGTGTGACTCGTGTGCTT
GAGTGACTATGCTAAGCAGGATGGGTCGGTTCTTCATTGGTTCATGTGAATTTGCTGCTGCTCCTGGCGG
CCTGGCCTGAGTGCTGCAGCAGCTTCCCCACCACCACCTGGCAGGATGAATGACAAACACCGTGCCATGG
CTCTCGGCCAGGATGTGGTACTGGCAGAAGACCTCTCACTGCAGTGAAACGCCTTTCCAGTGACATACAG
ATTCGTGTTACATGGATCCTGAAAAAAGATAAGGCATGAGGAAAGCTGGTCAAAGCCAGCACTGCTGCTT
GAGAAGGGAGGAGCCACTGAGAGGCGAACTCTCCTATTCAGAGCTTCCTGGCTCCATGATGGAAGCTACG
AGCACCCTCTCCACACCCAGGGGTGTTCTGCTTCCCCATGATTACTGGACAGGAACCCCTCTCCAGGGCT
CCATTGATGTGGCTAGAATTGGCTGACCACCTGGATGGAAAAGCCTTTCCTCTTCACAAGGGGCATAAGC
ACAGGCACGGCGTCTGGCCCACCCAGTAAAGCTGCCCACTGTTCTGCTGAGCCCAGGCCTGGCTTCTTGC
ACCCCTCCTCAGAGCCAACGTGGTCCTGTACTCACTCCAACCTCAGCCGCCCTGGCTTCAAGTGTTACCA
GCTGCGAGTCCTGGTCCTCTCTGACCTCGGCTGCCTCTTCTGTGCGGGGAGGGGATAATTTTGTACCTGT
TTTGTAGGGCTGTTAATGAGGATTCAAGTGCAAGCGAAACAGCATCATTCCAGCCACTGCTTTTCAGCCT
TTCCCACTCCTCTGCGCCTTGCCCGTTGGCAGCCTTTCAGGAGCACACTAGGCCCCACCTCTGGGCCGCC
GCCTGGGATCACTCCCGTTACCGCCAGTCCTCTGAAGTCCTGTCTGCAGTCCTGGCTTCACCTCCACTCC
TCTCCTCTCTCTGCCATGTGCTGCCATCCAGGGCCTGCCCGGCACACTCAGGGTTCATTCGCCCCTTTGT
ATCTTCTCGTGCTCTGATGACGCGTCCTCTGCTTCTGTGCCTCCTGCCATCTGCAGCTGCTCCTCCTCCC
CTGCCTGCCTCCACACTCAGAGCTTCCTCCCTTGGGGTTCTGACACCCAAATGTGTACCTTCAGCCAATT
CCTCTCCCGGAGCTTCCACCTTGGATATTCAAGGACCTATTGAATATCTCTGCTTGGTTGTGCCTCAGAC
ACTCCAAATCCACACACCTCAAACTGATCTCTTAGTAAATCCTCCAGAAATCATTTCTTCTCTTCTTTTC
ATCTGAGGAAATGGCATGTCCAGTATTGAGGGGGTGTGGCACCATGGCAGGGCTTTGCCAGGTGTGGGAG
GTGGGGAGATGAGGAACGGGGCCTGCCGGTGGGTCCTGGACAAATTGAAGCTCAGGGTGTGAGCGTGAGA
TCAGGAGGCTCTGAGGTGAATGTACCCTCCCTCCCTTCTCCCTTCCCCCTGCCTTCTGCCAGCTCCTCCG
CAGTCTCCCCGGGTCTTCAGCCTCTTCCCTGCTGCTCTTGCCCCACGGTCAACCCTGGACAAGGTCTGCA
GGAGCTACACCAAGCAAGCAGAAAAAGTGAAACCTCTAGGAACTGCTCTCCCCATGTCCCTTCCTGTCCC
TGGACCCAGTTGCTGGGCTTCACACCAGCTCTGCAGAGAAGTGGCCCAAAAGTCATGGTGAAGGGACAGG
CCTTGAAGGGGTTGTTGGGTCGGTGGGAGGCTTACGCCCCCACTTTAGTTTTCCTTATTAATAGCAGTAG
CCTTGTTTTTCTTGGGGCAAGACAGGAGGGAGATTAATGAATAGACATCAAGTTTGAAAAGCACCCACTG
GGAAGCACCCACTGTCTGGGATGTGCAGGTAAAGGGCAGGTGGCTCTCGGGGGCAGCTCTACTGTATCCC
AGAGCCAGCAGTTGGGGCTCAGAGACAGACATGTGCCCCTCATGCTCCCTCCAGGGAGGGGAAGTGGAGC
CCCAATTCACTGACACAAGAGGACCTTAATATATGTTCCTTTAGAAACAAATGTAGCTACACAAAATGGT
TGTGTCTGGGGAGTAGTGGGGCTCAGGCCTGTATTCCCAACATTTTGGGAGGCCAAGGCAGGAAGATTGT
TTGAGGCCAGGAGTTCAAGACCAAACTGGGCAAAATAGCAAGACCCCCATCTTTACAAGAAAAAAAATAG
CCAGATGTGGTGACGTGCACCTGTAGTCCCAGCTACTGGGGAGACTGATGCAGGAGGATCACATGAGCCT
AGGAGTTTGAGGTTACAGGGAACTTTGATCACACCATTGCACTCTAGCCTGGGTGAGAGAGCACGACTTT
GCATCTTTTTTTTTTTTTAAAGGAATCTTTGATTCCCAGTCCTGCTAACCTATCCAGTATCACACTTAAG
GTCCAGTGCATCAACTAGGCCATAACTACCATATATGAAATTGGATTTTTTTCTTTTGAGACAGGGTCTT
GCTCTTTTGCCCAGGTTGGAGTGCAGTGACGTGAACACAGCCTCCTGGGCTCTAGCGATCCTCCTGCCTC
AGCCTCCCAAGTAGCTGGGATTACAGGTGTGCTATCACCATGCCAGGCTAATTTAAAAAAAAAATTTTTT
TTTGTAGAAGCAGGGCCTCACTATTTGACCAGGCTGGTCTTGAACTCCTGGGCTCAAGTGATCCTTCTAC
CTCAGTCTCTCAAAGTGTTGGGATTATAGGTGTGAGCCACTGCATCTGGCCTTGAAATTGTTAGAAATAT
TTTCAACTTTCAAGCAACCGATTATAATTAGTTATTGGATATTTATGTGCCAAGCACTGTGCTAATCACT
GTACGTGTGTTGTTGCATTACAAGAACACTGTGAAGGAGATAGGCCCATTTTACAGATGAATAAACTGAG
GTTAACTTCTCTGAGATCATACAGTTAGTAGGCAGTGGCGCTGGGACCGAAACCCAGGTCCGCATCCCTA
TTGTGAGGCATGATTACGCGTGTTTGAACATGCCTATTTCTTCAGTTAGGGACTGGGCATCACGAGGCCT
TTTCCATCCCGTTTTTTATCGCTCCCTCTTTCTTCCATCACTAGACATTTGCTTATTCTTTTTGAAAGCA
ATTTTTTTCCTGAGCATTTCTGATTTCTTAAGGTGAGTTGAAGTGGAAGCATATTCACATTATTTTGCAA
ATGAGCACTTGGTAAATATTCACTGATTCTATAAATTATTAGCCACTGCTGTGACCATGATGAGAAGTCT
CACAAATTGGATTAGTGATTGCATTTTGAACTTTTTTTAATCTTCTTTTTTCAGTGAGCCATTTCTGTTT
AAAATTTTGTTTATTTCTTTCTGAAAGGAAGAAGATTTCTCTCCCCATGTCAGCTGTAGTCTGCCTCTGG
CTTCGGCACCTTCCCAGCCTTGAAAAAGCAATGCTGCATCTTTTTGAAAAGCTAATCTCCAGTGAGAGAA
ATTGTCTGAGAAGGATCGAATGCTTTATAAAAGATTCATCGCTGGTACGTACTGGGTTTTGATGAAGGGA
AAAATCCTTGAAGGAGATGCTTGGAATCATTTCTTTTAGTCAGAGAATGAAAGGGGGGGATGTAAACAAA
AGAGCAATTGGAACCTTTATATATAATCGTGTATTTACATTTTATTTTTGAAATCACTAACATAGGGGGC
TTTTCTGCATCCTTGGAGCATGCAGGGAGAGATCTGAGGTTTACTTGATTGATATGCCGTATATCACATG
TTCACAAGAATTGACATTTGCACAACAGACTTTTTAAAAACAGTATTTCTTATTTCAGCATGGAAAATAA
GTTTTTGGAAATGAACATATGGGTAAGAGGAAGAATCAATTAAAAACATTCAAAAAATAAACTTGTAGCT
CTTCACTAGAGGAAAGATGAAATAAGAGACACAAAACCCCAGAAAAATGGTTAAAGCAGCAGAACCAGCT
AAGACTTCTGTAAGTTTTCTTTTGGTGTGAGGCCACCTGCCTTCACCCTCCCCTGCGCGGTGGCGCCTGG
CTCCCTTCCCGCTCAGGACTGTGCAGCTGGGGACTCCAGCCCACGTGGACATCCACCTCCTGGCCCAGTG
TCTGGCACCCTGGGCTCTCAGTGGCCCTCGCCACCACTGACTCGCCAGTCATATTCCTTTGTGGGAGGCA
GCTCTGTGGGATTGGAGCACATTTTGTAAGGACAGGAATAAAGACCAAGGCACCCTGAAAAAATGATTCA
GAAACCTGCAGGTGCCATGGTCCCGCAGCCTCTGTCTTCCAGGAGCTCCAGTGAACTCTGCCCTTTTTCC
CTTTAGCTTTCGTAATCTGCAGGGATGTGGCACAGAGATTGCCTTCCACTTCTTCCTGCTTCCTGCAAGT
GGAGAAAGTGAGTTCCAAGAGCCACAGTGAACGGTCCGGTCTTTAGTTAGTGGCCGGGACACTGGGAAGG
AGGGAGTGTTCACCCTCGCCCTTGAGCTGAGACTGAGGAAGTCCCTGAGGTTGGCTGTGCCCACTTTTCC
TGAGCTCATAGAACCCCTACCTCCTAGAGGCCTTCAACTTGTTTAGTTCAGTTGGGACTTCTGTGTTCTT
TCTTTCTCTTTCTAGTTGGAGACTGTCTAAGGGAATCCTAATGGAATCAGTTGCCTTCCTCCGCGATCCC
GTTTCTCCCCTTGTTTCATGGCCCCTTTCACCCTTCCCCTTGGCCTCCCAGGGCGATGTGCATAGGGTTG
CAGGTGCTGACAGCCACGCTGCTCTCACCTGTACCTCAGGCTGCACACACGGATCCCACCTGACTTGAAA
GTTTACCACGATAGAAGAGAAAGGAAGACGTGTATATTAAGACTGAGAGAGCCCAAACAGATTTCTTAAG
GATAATTACTACCTAACCTTACCATCCGGAAGAGTCATATATTAAGGCAAGAAATAGGAATGCACCTTAT
TTTAAAATGGTTGCAGTACAATTCAAAATAAATCATGACTTTCAGAAGCCCTCTGACAGCCCATTCCTGG
TGATTCAAGGTCATAAATTACCTTTATTGTCTGCTAAATCTATTACTAACTTACATGTGCATACTAGGAT
TTCAGTTTCTCTGGTCCCTTTTCAGTGGCAAAATGATAGTAACTGGGCTATATGCTTGCTTCCAGGGTGC
TTATGACATTTTCCTTAAGTCTTCTCCCTATTGAAAAATTCTAGTCCCATTGCCTTTTTTCCAGCCTTTC
CTTAATCCTTGGATTCTTCCCTGAACCTTCTCCATGTCCTTTTTGCCTGTGGCCCTCATGCCTGAGCAGC
ACTGTGGTGAGCACAGCAGATGCAAGACAGAGAACGCAGGCCGTCCCATTGCACATAACTGTATAATGCT
CAGTCTCCAGTTTGTAGTTTACAGTAATCTCCCACGAGTCACACAGCTTTGAAGTCAGTTGACTTACAAA
AGCTTTTATCGGGATCATGTGTGCAGAAATTCAGAGCTTCCATTATGGCTAAAGGGGGATTCTAGAGGGT
GTTATTTGGTTTAATTTATTGTGGCATTGGCAGGATGTGACCTCTCTGTGCCTCATGGTTCAAGGACGTA
ATTACAATACAGACGATGGCTGGAAGCCCCTGATTGGTGTGGGCTGTTCTCCTCAGCATCCTGGGCCTGC
AGTGCCAGAAAGCCAGGCAGGCCGCAATAGGCCTGTGACGTGGCTAAGTGATTGCAGGGGTAACCTGAAG
AGCCTGCTTCCTAATTGCATCATCTGATTCATTTATCGCAGTGCAGTATCTTGGTGCAGTGACAGGACTG
TACAGAAGTATCAGAGAGATCTCTAAGACTCAGATAGCACGAGACTGCTGCGCTGTGCTCCAGAGGAAAC
CTGATGCCTATTACATTAGAGTTGGATTCTGAGAGTAATTAAAGTGTGATGGTCTACATAACTTGTGTTT
TATATAGAGCACTGTTAATTATATTTCTCTCTCTTGGGACTATGCAGAATGAGTCCTGGAAATTCTATAG
TTACCAAAATGTAACAAAAACAAAAGATAAAAGTGTGGTACAGGTTGTACAAAGTGAAATGACCTTCCCA
AATGTTCTTTGGCCTCTTGGACTCGCAGGTCTGATATTTTCCATTAAGACCATGAATAATGAGGGATTAG
TCTCTTATTACCTGAGTTCCAAATGACTATGTATGTATTTAACATCTAAAATAAAAGAAAAGTTTTCAAA
ACCCGTTCACTTTTGAGGCTTCCCCTTTGGCTTCCGTGCGGTGGACTGGACTGCTGTCGAGTCATGCTGT
CTGCCACGGTTGGTAGCTCCTACATCAAGTGAGGTATTAGCCCTAGATTGTCATTTACTGTACAGACTGA
AACTTTTCAAAAGCAGATGGTCTTGTGTTTTCCTTGACAGCATGGGCACGGGAAGACCATAATCCATAGC
TTCAGACCTGACCTTAAAAACCGATGTCACCGACAGCAGTGAAAGCCACTTGTTAATTATGCAGCCTTCT
TGGCATTGTTCGGTTTCCCAAGGCTGGCCCCAAATTAGAAGGATGTTATGTTTTCCAGTTACAATTTTGT
TTGTGGAGCCCAGAAAGCTATTCTTTGGATTCCACATTGGAATGAAAAGTGTCTGCAGGGTGTCAGGGTG
GCACTGTGCTTGGCTGGCAAGCTGCATGCCAGGGCCGGAAGGTACCCGATTCTCATGCCCTCCCATTTGC
TGGCAGAGCCCGCCCGCAGCTGGGTCCAGAGGAGCTAGCCTCACCTTCCCAGGCAGACTTGTCCCCAAAA
GAAAGGAGCAGAGGAGGCGCCCAGGAGGCAGAGTTGTTTCTGCTAAGATTTGATATCAGAATATGCTTCA
TTTTCTCTCCTACCTAAAAATCTTTATGGCTTAATTCAAAAGATTACTTTATCTAATAAGAACTTAAGAA
TAAAAAGCATCTAGATACCTCAGTATCCTCCTCAATATGGGATAGGTTGTGTTTTTTTCCCTCATTCTCC
AGGTAGCAATTGTAGCACTTCAGAAAGAAAAAGAAACTTCCTTTACACAGACTCCCAGTATGCAGCTCCT
CCTTGGTAAAATATTGTGCTGAAGAGTTCACTCTACATCCTGGGTGATATTGTCAGTTTGCTGTGCTTAA
GTAGCTTTTCACTAACATGCCAGGGTTTTCTTTTCCTGCTTTTTCAGTCTTTTCGGCACTTCCTTTTGAG
CTCTTTAATGTGGTGCATGTAATTGAACAAGAGCTGAAGTAGATAATTTAGCAGTAGTCCAGTAGTGCTG
GCTGATGTGTATGGCTAAAAGCTGGGAGGTTCTGCATCTTGGACTGGAGTTGCACTTTCCAGAATTAGCC
TCAGGGGGGCACCAGATCCTCCAGCATAGTCTCTGTGAAGAAAGAGGCCCACGTTGCGAGTTTCCTTGCA
ATGGAATTGCAGTTGGGGATGCAGACCTTGTGGCCAGAGATGAAGGGAGAGAGGGAAAAACCCAAAAACA
AAGCAGGGGTGGACGTAGGGGTCATTGCCACTCAGGCTGAGCTGTGAAGCAGCAGGGGGCTGAGGAGCTT
TCTTCCAGCTTGGGCTACCCGGGCTAATTGGATAGAGGCCACTGGGCAAGGCTAGAAGGACTTCAGGCAG
GGGAGGGGGTGCTGGGGATCCTCTGTGTCCAAACACAGAGAAGAGCACATGTAAATTTAAGACGCAAAAG
TACATTTGGTACTTGTTAAGTTCTTAAGCCCAGGAATGACAGATTTATCACTAATTAAACAATAATTGCA
TTATGGAGTACTGAAAAAATTGTTTGTGTATAAACAACATCTGGTAAAGATTAAAAAGAAAGAATAATTT
TTCCGAGCTCAAATTTGTTAAAAGGAACCTGAATGTTTTAAAACAAAATAGATCTGAAAATGTAAAAGGG
ATATTTAGAGTCTTATGTTTAGTATTTTGAAAATAGAAAAGAAGCAAATGGCATAATAAAAAATACATAT
TTATAAAGGTGCCTCATTGTACATGATAGATGTGAAGGTTTTGTATAAATAGGTTTTTCTCAAGCCAAAT
CATTTTTCACTTCAATAGAGGAGTTCACTGGTGAAATGACTCTTATGGAGAATCTTCTTTTTAAGAAGAA
AGGTATTTTGTAGTGAATATGCACATATTAACAACAAGGGCTTCCCACTGTGGAGAGAACAGTGGCTCGG
AAACAGCTACACGGTGGGACGGGATTGTCCGGCTGCTGGGTAAACTGGAGGGGAAGCTGCCCATCAGCTG
ATTCTTTAAATGACCCATTTCCTAAGTAAATATTTAAGCTACATGCAAGTACATGAAAATACCAGGAAAG
AATCTATTCCAGGCTTAATGAACTTCAGCTTCTCTGTCATCTGCTATTTCCTTCTTTATTTTTAAGGCGA
ATATATCTATGAAAGGTGATAGATGGAAAGCACTTGATAATTTTTTCTGTTCCTTCACTGCCTTTTTATT
TTTTCTAAAAGGGATTTGCTTTTGTCTGTGAGTTACCTTAAATCATAGTATAATTTCCCTTTTTGGTTTG
TTTTCTGTCCCTCTCCTTTTCCCTATTCTCTCTCTCTCTCTCTCTCTCTCACACACACACACACACAGAA
TCAGCAAATGACAGAAAGAAATGCTTCTTCCTGAGAATTCTGTTCTTCTTGTTCTCAGGCCTCTTTCAAG
TGGCAAGTGGCTTGTAATAATCATTGGTGAGTTGGTATGAAAATAACAGCTTAAGTAAACATCAAGGACC
GGGCACAAGAGCCTGTGTAATTTGCACAGTTCATGGTGCAGAGTGACAAGTTGGCCTCTCCCTTGCCTCT
CTTCCAAAACAGGCACTGAGACCATGGTTATTGTGTGTTGATTCCATGTTGACTTTGTCTTCCCTTGTGT
TTTGAGGGATGAAGATGGGCTTTCTTTCTAGGTGGAGGGGAGATAAACAATGCAATTCTGGGTAGCCATG
AGGGGAGATCAGAGGGAAAGCTCTTGTTAGCCCAAGGGGATGCACACCCAGCAGAACAATCGGGTCTATA
CAAGAGGGAGGGCAGAACCCAGAGCTCTCCATCTTCGCTCCCTAGTCATCCTGGGCATTCAGTGAAAAGC
CTGAGGGGGGAGATAAGGAGGGAAGGACAAGACTCATGTTATTGAAGACCTCCTGCTGACATAGCTCCCC
TCGAGTTGAAATGATCTGCCTGTGGAAATTGTTAGGCCAGATGGAGAAGAGAGAGCTTGAGAAGGAACAG
ATTTTGGTAGGATGGAGAAAAACAAACAGATGGAAACCACAAAAGGTTTATTTTATTTCCCCGCTTTTAT
AGAAAAGATAATGGCCGCAGTCATGGGGCTTCAGAGCCCTGGCTTGCAGCGTGACCTCTGGTGTGTTACC
GGAGTGCCCCACCTTACAAATAAAACAGGATCAGAGGTTAGATGCCTGCGTCATCACTCATGAAGATGCT
ACAGAGATTTAGGGGGGAAGATACGGCTTCCAAAGAAGCGTGTATTAAAACCTTAATTCTATTTGGAAGA
TTATCTTGATAAGAAAAGCAGCTTAAGATTGCTTTTGTGATTTGATGTCGCTGGGACATATGCCTGACCT
GTAGTACTCTAGTCCTCATGAGGGGACTCACTGACCCTGAAATTATGTGCTCAAAAAGTATAAAGAGTGT
CATTTTAAAAGCCATCGGTGAGGGGTGCAGATGAGAAGGAGCAGTTCACAGCTGTAGGGGTAGCAAGGGA
GACTGGTTTTCTTTTTTTTCAGTGAGGTCAGGAGATCGAGACCATCCTGGCCAACACGGTGAAACCCCAT
CTCTACTAAAAATACAAAAATTAGCCAGGTGTGGTGGCACGTGCCTGTAATCCCAGCTACTCAGGAGGCT
AAGGCAGGAGAATCTTTTGAACCCAGGAGTTGGAGGTTGCAGTGAGCCAAGATCACCACTGCACTCCAGC
CTGGCAACAGAATGAGACCCTGTCTCAAAAAAAAAAAAAAGGAGACTGGTTTTCAAATCAAAATCTCTGC
CGTTGCTGTTTCTGCAGGTTCCCCTGCCTCCTGGCCTCTTACCTGTTTTAAAAGCTAAAATGGGTTGTGG
AAGCAGGCTGAGAGTAGCCAAGGGTCTGAAATATTTGGAAAGAAAGAAATCTAAGAGGAGAGTCCATCTG
TATGGGGAAGAACAAGCAGATTCCACACAGCCAGCAGGCATTTAAAACATTGGATTGAGCGATTTTGCTA
GAAAACAGCGCACAGAAGAGGAGCAGATCTAAGCTCTCACTCTCTTCTAACAAACTGAGTAAGAGCTGTC
TTCCTTGCTCCCCCAGTGGTTTTCATAGTAGCCCTAATCTACATCTGATCATCATTTTCTTTGCTGTTTG
GAAATCCCTATAATTAGTATTGAATGATTCTCTACTAGCATCAAACCTATAGAATGGCTCTGGGATAGTT
TGACCATTTAGAAAAAAGAAATTTAATTTTACCATTTTCAAGTTTCATCTTACTTTCTATCAACGCATTT
CCAAGCTCTTATCTGTCTGCCTGTGGGCACATAACAGTGCTATTTGATCTTAAACACTCTTATTCAATAG
GCATGTTGTTGTACAAACTTTGAAAATTGCTCTGGCAAACTTAGATGAATCACATTTCTTCAAGACATAC
GGTTGCTTTTCCCTTGTTTTATTTTTTCATTATGAATACATAGAAACTTTTATATTTTCCGTGCTTATAT
CCGGTGACCTCTTAATAACTTTTATTATTTACAGTGAGACTGGGCATCAGGAGATTAACTGACATGCCTA
AGTTCGAAGCCACGAAATAACCTGCCTCTCCTCTCTTGAACGGTGGTCCTGCCAAATTATTTTCTTGCAT
AGATAATGAATTTATCTGTAAGAATATCAAGTTGGTTCCTTTTAGATAAAAATGCACATCAGTGAAGGAT
GAGGGAGCATAGATTATTTAAAGAGTAGGACATTTTAACTTGCAAATTAGAAATCTTGAAATAATTAAAA
GGAGATTTATCCTGTCCTGAAAAAGTGGCAGAGCTCCTGCCTGGAGCCCGGCGGGTCCAGCAGTGCTGAC
CAGTGGACGCTAGATGGCGCGGGCTCAATGTCATCGGGGAGGCCGCGAGGAGGCGCGGTCCTGCCTCAGT
GTGATCCAGGGCACGGCCGGAGCAGAGCGCCTCCTTTTTTCTTGCCGACGGGGGCCTGTGAACAGTGTTT
CCCAAAGCTAGTCATATCAAATGCACTCTGACTTGAATTTCCAATAGCGCCTGATCCACTAACATGTGAA
TGCTGCTGTGAAGCAGGGGTCCCCGCCCCCTGGGCGCACAGCGGAGAGGAGCCCCTAGCCTTCCCGCCCG
CACCTGCCCCTGTCAGAGCCCCGCGCTTCGCTTCCCACGGAACCAGAGCCCCATCCTGACTGCGCATGCC
TGGAATCGGGTTGTCCTCTGATGATCTGAGGAGCAACCGTTTCATCCCAAAACCATCTCCCCCTCCCGGT
CTGTGGAAACATTGCCTTCCAAGAAACCGGTACCTGGTGCCGAGAAAGTTACGGACCCTGCTATAAAGGA
TCTGTTTTGGAACACAAATGGGCATTTTCATCTGGCATCATCTCATACTTTGAGCTCTGGCCAAAAAGAT
AGGGAACCAGGGACTGGGCCAGTGAAGCTCCCCCCAGCTCCCAGGGCTACCGGAGGCCAGCTTCTGCAGA
CTCAAATAATCAGAACCCATCACCGAAATATTCAAGGAGAAATTAGATAACTTTTATTCTTCTTCATGAA
CCCCCTTTTCATCTTAACGCAAGCTGTTAAGTGCATTAAGATGTGATGTGATTTAGTCCAGCGTGGTAGT
GGTCACTGGTTTGCCAATAATAATTCTAAATTGTTATGTACCCATTACCGAATAAACAGGCTGCAGGCAC
GGGCCTGACCTCGAGGCACATCTAGCCATTTGAGGAGGTCACACAGGCACACTTCAGTAGAACTGGCAGT
ACAGGCAGGCTCCACGGCATGCGGGCAGAGGGATGTTTAAAGTTTCTCTAATACTTACTGATTCTGTATT
ATGGATCGCCTGAGCTTTTATAGAAAATGTGGGACATACAGGAGTATACAGAGGAATAAGCTGCCTGTAA
CCCCTGCCCAGGGAGACCACCGTATGCACTTGTATGTGGGGCGTTTTCAGTACAGCAGTGGTTCGTTCTT
CTAACCAAAAGGAGTATGCCAGTTTTCTGGACATCAGCAGTTCCCAGTAACTCCTTTGGCTGATAATAGC
AAGTTTCTGAGAAAGTGCTTGTGATATTTCACATTCTCATGGTCTTCTCCTTTTACAGCCTCAAGCAGCC
TGCCACCCTGCCATATTCCGGGTTGTTGATGAGATGTTCAGGTAAACGTTACACTGTTTCTTCTAGTAAT
TGATGTAAAAAAGGTTCCATTTCCAAGCATGAATCAGAAAATGTTGTGGTAGTCTCTGGCTGTATCATGG
GGAAATTAGAGTTTCTGATTCCCTCCTGTTCCTCTGAGCTTTTTGGTAATTAGAGGTAAAAACTCAAAAA
TCAGTGTAAATTTAAAGGCCAGTGCCTAAGAATTAAGATAACTTTATTTTCTTTAGCCTTTTAAGATTCT
AATAATAGAAACACAGAATTGAGTTAGGCATTAATATCATGAAATATTCATGTAGGATTTTTCTCAGCTC
AGAATAAAGGGTAGCAATATACTCCCTTCACCATGTGAGGGCTGACCAATATTTTTAAAAGACTTTAAAG
CTAAAGAAAGCCAAGAAAAATAAAAGTCATTCTTAACAGAACTCTTCTGAAAGGCAAAATACACCATGCA
TGTTCTCTTACAACACATTACATTTTTATTTTGAAAATATGAGTACTTCTTGGGAAATGCATTAGATTTA
CAGACGGCAAGAGACGAGGAGCTCACAGCCATGAAGCTGAGATGTGGCTGGCAGTTAGTGGGAGTTTTAA
CCATGGAGTACTAAGTTAGTGGGAGTACTAACCATGGAGTACTAAGACTGGACGCCCCTGGGGAGCTTGT
CACCTGGGAGTGTCCCAGCCCCTCTTCCAGGGGAGCCCATATCAGCTCTCTCTCTAGCCCCTCCCACCTA
ACACACAGCAGCAGAGGCTGCAGGGCAGACTGCAGACTTTGGGGAAAGGAATTTTTACTGTGACAATAAC
TCACCAGTAGTCAGAACAGTGTTGGGGGTTTAAAGCTAGCAAAATTTGAGAGTTTTTTTGTTTGTTTTGT
TTTGTTTTGCTTAAACAAACATAAAGAAAAATTAATTGTACAATAATGCATGGAAGCGAAACCAGGTACA
CACACATTATTTTCCTACTTTGCTGGGTGGATGGACGAGGCCATTTTACCCTTTGAGCAGCTTCGTCTCC
ACTTGTGAAAGCAAAATGTTGATGTTGCGTGTCCAGGCATACAATACAAAAATATTTGTACAGAGTATGC
AAGCCTGTGTGGAAGGGATTTGCATTGCTTAATAATTTAAGAACCAAGAAAGAGAGCTCTGCACGTTGCA
ATTTTAAAGAAAATATAACAGGGAGCGACAAATTTATCCTGTTTTCACACAAGGACTGAAATCTGATGAG
TTGAGATACTGCTGAAGCTTATGGCACAAAAAAAGTGTTTCTACTTTTCCCTTATACAGTGCAGGTTTTC
ATGTTTGCCGGATTACTTGTTAAACGTGTTCTGATCTGACTTTGCATTGTTCAGGTGTGCACTCCTGGAA
ACCGATGGGGCCCTGGAAATCATAGCCACTATTCAGGTGTTTACGCAGTGCTTTGTAGAAGCTCTGGAGA
AAGCAAGCAAGCAGGTTTGTTATATCACATATATTACTCATTCATCCCAGAGAATAAGACGCTGTTGAGA
GTATTTTGGACAAGAGCACTTTATTTTCAATAATTTTGATGGACTGTTTTGCTCACAAAGAAAGGTTCCT
AATGAGTGCCCCAACAAAGGTTAAGCCATTTTAACTTAACATAAATCAAGATCTGTCTGCCCCGTGCCCT
GATACCCAGAAGCATCTGTCTACTATTATGTCTGAGAACTCTGTCATGCCAGCATGCATGGCCCTGGAGG
ACGGCTCTTCTTGGCCCTGTGTTGTGCACTGTCTGCCGAGCAGGACACTGCCCCCTGGCTGCATCTGAGC
CTCCCAGGTCAGCAGGAGGCACCCTGGGGTCTATATGGCATGTGATGAATAGGAAAGGGCACTTATTACA
AAGCACTCCTGGACATTCTTAATTATTAGCATCTTCATTCTTATCAGAGACCCAAATGTGAATTGGCTTC
CAGGAAAGGAGGTGGTAAGAGTGACTGGGAAAGGCTTGACTAACTAGAAGGGGAGTGAGGAAGGGCAGTG
AGCGGGAGGTGTGTGTTAGACACAGTCAGTAAACCAAAGGCCAGGCCCGCACCCTGCAGAAGATTGTGCT
CCACGTCCCCAGCAGCAGAGGCTGGGCAACAGCTGCCAAGAGTCAATGACCAATTAGAGGGACCTCGTGA
AACAGGGACCTTTGCTACCAGCGAGCTTTCACCTTTCAGCATATATATTTGGCCTTGATTTCTAACGGCC
ACAGAAGTAATGTTTATGGGACACATTGCCTGTCGAGAGTGGTGCTGGTCCAGTGCATCTCATCTGTAGA
GTATGTCACGGCCCCTGTGAAGCTCTCCGCAGTTTGACTTCCGAGAGGAGAACTACAATGCTTGGCTCCA
CCAGATCACACCCCCCGCCAACGTCAAATAGATCTCTCAGGTTTTAGCCGATATTTCTACTTTTTTTTTT
TTTTTTTTTTTTTTTTGAGACAAGGTTTTACTCTGTCACCCAGGCTGGAGTGCAGTGATGTGATCTCAGT
TTACTGCAGTCTCAACCCCCCAAGCTCAGGTGATCCTCACACTTCAGCCCCTGCGTGTAGCTGGGACCAC
AGGTGCATGTCACCACTCATGGCTACTTTTTGTATTTTTTAATTAAGTACAATTTCCATTTTATTTTTCT
CAAGAGAATAGTCCGTCTTCAGACTTTAAGGACTCAACTCCTTACGTGGGCTTTGGTGGGGGTTGTGGGG
CAGCATCCGCAGGTCTAAATTGGAGTAGGGGTGTTCAGTCCTTGTGGGCTTCACAAGATCGATTCCTGAC
TACTTTGCTGTGAATTGCACAACTCAGACAGTAATGTAGCTTCAAATACAGCTTGGGAAGCACATAGGCA
TCAAAGACGCTCGCTTCAGAAATGTCCCTGACTGCTGCAGCCTCCACTGTGTTTCCAATGATGAATTTGT
TAATAGCCTTGTCCTTGGGCACACATTGGGCACAGTTCGTGTCGCAAATAGGCTGCACGTGGCCGCGGCC
CTTTTTGGTACGACCGTTGTTCCTTCTTTTTTTCATCATATTGGAGGCACGGACTGGAGGAAGCACTAAT
TTTTGTATTTTTATTAGAGATGGGGTTTCACCATGTTGCCCGGGCTGGTCTCAACCTCTGAGGCTCAAGC
AATCTTCCCACCTTGGCCTCCCAAAGTGCTGGGGTTACAGGTGTGAGCCACCACACCCAGCCTGTTTCTG
CTTTTTAAAGCAACAATAAATATATTTTTATTTTATTTTATTTTTTGAGACAGGCTCTTACTCTGCCACC
AAGGCTGGAGTGCAGTGGTATGATCATGGCTCACTGCAGCCTTTGGCTTCGTGGGCTCAAGCGATCCCCC
CACCTCAGCCTCCCAAGTAGTTGGGACCACAGGTTCAAGCCACCACGCCCAGCTAATTTTTAAATTTTTT
TTTGTAGAGATAGAGTCTCTCTGTTGCTCAGGCTGGTCTCAAACAGCTGGGTTCAAGGGATCCTCCCACC
TTGCCCTCCCAAGGTGCTTGGATTCCAGGCTTGAGCCACTGTGCCTGGCCAACAGTAAATATTTGTGATT
TATTTTTTCCCACCCTCACTTTCCCTGGAGAAGTTAGCTGGGCTGTAGCAAGCTGCGCTATCCATGGTCC
CACCTAGAATTTTGGAGGTGGCTCGGCTGGAAGGCACAGCTGGCCCTCACGGTTATTTTGAGCCTAACTG
TAAATGTCTCTGAGCCTCCCTTGTGCATTCTTTGTAGACCCCATTAGGAACGGGCCTCTGGGGAGTCTCA
CCTGTGGCCTCGCGGAGCACACCTGGGTTGGAGCAGAGGCCACGGAGGAGTGCCACTGTGCTCTTTAGGG
TTAGCTCGCGCCTCTTTTTCTTCTTAGCTTCACCCTCAGCCTCTATGTTGGCCGTGGGCACTATGGATTG
GGACTGATGTTGCAGTTGCTTTTAATTGAGACTAGTACCCTGACCAGGCATACGTATCTAAGGACGTGGG
ACCACACACTGGGTCCTGGTGTGAGGCAAGGCAGGGATCCAAAAACAAACAGGAATTCACCACCCTCGCC
TTCAGCTCAACAAAGGAAAGGAAAGTCTCTTTGCTTTGCCAATGCCTGCATAGGTATCTGTTAGTTTATT
TCTCTTCCTAGAGCTGGACCTGAGTGCCTGTCTTTTTAACTGATGGCCATCTACATCTTTTGACCCTCTG
CCTTCTTGACTGTCCATTTTTAGCTAATCAAAACCCTTCATGTCATATTTGGTAGATGCGTGACCCTCTT
TTCTCTCTGTGTTTTATTCATTAATTCCTTCTTAATAAAATGAGTATCTGTAAACCTGATATCTAATAAT
CTGAATATATGTCTATCCACGTGCTCCTCCCTGCCTCTCTCCCTCCAGGTAACCTCTGGTTTGAATTTTA
TGTGCCAGGCGTGGTGGCTCACACCTGTAATCCCAGCACTTTGGGAGGTCGAGGCAGGCAGATCACAAGG
TCAGGAGATCGAGACCATCCTGGCTAACACGGTGAAACCCTGTCTCTACTAAAAATACAAAAAAATTCGC
CGGGCATGGTGGTGGGCGCTTATAGTCCCAGCTACTCAGGAGGCTGAGGCAGGATAATGGCATGAACCCG
GGAGGCGGAGCTTGCAGTGAGCTGAGATTGCGCCACTGCACTCCAGCCTGGGCGACAGAGCGAGACTCCA
TCTCAAAAAAAAAAAAAAAAATGAATTTTATGTTTATTATCCCTTTGCTCTTTCTGAAAGTAGTTTGATC
ACATATATATGACTGCCTAAACAATGTTTTGTTTAGTTTTGGTTGTTTTTGCACTATATAAAACAAACAG
GAGCAGCATACTCTGTGCAATCTTTTGAGACTTGCTTTTCCACTCAGCAGAGACTTCTTGACGTTGCCAT
GTGTGGCTGTGGGGAAGTACTGTTATGCATAGTCCATTGTGAAAACGTATGGCAGTGTAGCCATCCTCCT
GCTGGTAGGCACTTGAGATGCTTCCAGGTTTTGTTTTTTATTACAAACAATGTTGCTCTGGACACTCTGG
GATAGATACTTAGGAGTGGAAGTGTATTTGGAATACAAAAGTTCAACTTTCATAGATCCTGCCAGACTCA
TTTCAAAAGTGTTGGTGCTCATCTGCACTTCCTGCAACAGTTTATGAGTGATCCCATAAATCCACATTCT
CTCCTATCATCAGAAGCACTCATGTCAGTGCTTCTGTCTCTTCTTACCCTTTCCTCTTGTCCCTGTTCTG
TTTGGAGGACACAGTCAGCTCCATCAATATGCCCTATCAGACAGTAGGCAGAAATTGAAATGGGTGTGAC
AAGCCACTTTTTCTAAGCCTGCACTGTGGCTCAGTACAGCCAGTCAGGTCAGTTTCTGGTTGCAGCCATG
ACAGCATCTGGTAATGAGCTAGATTAAATCATCTCCCACAGATAAAAATGATAAACTCCAGGCAAAGTAT
TTTTAAAAATCTATTTGAAGGCACTACAGAGCACCTGAAAAAGTGGACAGAAACTGGAGGCCATCAGCCC
TTTAAAGGGACATCACACAGAGAGGTCTCCTGCCTCCCCCCAGCTGCCCTCATGTGAGGTTGGAAGGCAG
GCAGTGTGGGGCCAGGAGAGGGCCAGAACTTGGCACAATCTGATTGACTTAAGGTGCCAAAAAATAGAAG
TGTAAGCTGCCAAAGGAACAAAAAAATTGAGGGGAAAAAAGAAAACCAATGTCAAAAGATAGACCTAAAC
CCAAAACTACCAACAATGACATTAATTTCAGCAATTAAATGGAAGTTAAATATTCTAATAAAAAGGCAGA
AACGTCAGATTGAACAAAAAAAATGAAGACCTGGCTGGGCACGGCGGCTCATGCCTGTAATCCCAGCACT
TTAGGAGGCCAAGGTGGGAGGATTGCTTGAGTCCAGGAGTTGAGTTCAAGACCAGCCTGGGCAACATAGT
GGGACCCCAGTCTCTACAAAAAAGTAAATAAATAAAAAATTAGCTGGGCATGGTGATGCATGCCTGTAGT
CCCGGCTACTCGGGGGGTGAAGTGGGAGGATTGCTTGAGCCCAGAACGTGGAGGCTGCAGTGAGCCATGA
TCACACCACTGCACTCCAGCCTGGAAGACAGGGCGAGATGCTGTCTTAAAAAAAAAAAAAAAAGATTTTA
AAAGCCATTGTAATCAAGACAGTGAGGTATTGGCAAAAAGACAGCCATAGATAGATCAGAATTGAGTCCA
GATATAGACCCATACATATGTGGCCAACTGATTTTTGACAGAGATGCCAAGACAATTCAATGGGGAAAGG
AACGTTTTCAACAAATGATGCTGGAATAACTGGATGTCCATATGTAGAAAAATGAACCTCAAACATTACC
TTGTAAAGCACACAACAGTTAATTTGAGAGGATCATGGGCTTCAACAGAAATGTTAGAAAGATAAAGCCC
CTAGAGAGAGCATAGGATAATGTATCTGTTTGACACTGAGGTGATCTAAGATTTCTCAGAAGCACTAACC
ATAGGAAAAAATGGATAAATTAGACCTCATCAGAATTTAAAACTTTTAGGCTGGGGGCATAGCTCACTCC
TGTAATCCCAGCTCGTTGGGAGCCCCAAGGCAGGAGGATCTCTTGAGCCCAGGAGATGGAGACCAGCCTG
GGCAACAAAGTAAGACCCCTTTTCTACAAAAAATACAAAATTTAGCTGAACGTGGTGGCACACACCTGTT
GTCCTAGCTACTCGGGAGGCTGAGGTGGGAGGATCACTTGAGCCTGGGAGATCAAGGCTTCAGTGAGCTG
TGATCATGCCACTGCACTCCAGCTTGGGCAACAGAGTGAAACCATATCTCAAAAAATAAGAAAGGAATGT
AAAATTTCTATTCTTTGATAGATATCATTAAGAAACTGAAAAAGCAGGCCAGGCACGGTGGCTCATGCCT
GTAATCCTAGCACTTTGCAAGGCTGAGGTGGGTGGATCACGAGTTCAAGAGATCAAGACCATCCTGGCCA
ACATAGTGAAACCCCGTCTCTACTAAAAATACAAAAATTAGCTGGGCATAGTGGCATGTGCCTTTAGTCC
CAGCTATTTGGGAGGCTGAGGCAGGAGAATCGCTTGAACCTGGGAGGTGGAGGTTGCAGGGAGCCAAGAT
CACACCACTGCACTCCAGCTTGGCAACAGAGCAAGACTCCATCTCAATTAAAAAAAAAAAAAAAGAAAAG
GAAGAAAGAAGGAACTGAAAAAGCAAGCCGTTAGCTAGGAGAAAATATTTGTAATATATGTATACAACAG
AGGACTTGTGTCGAGAATATATGAAGAGCTCTTATAACTGCCTAAGAAACCAGTTTTTAAAATGGACAAA
AGACTTGAATATATACTTTCAAAAAAGATACATAAGTTGGCAGTAAGCACATGAAAACTGTTCAGCGTCA
TTTGCCATCAGGAAAGTGCAGAGTGAAACAGGTAGGAAAGCAAGCATACATAGGGTTATGTTCCCAGCCA
CTAGAATGGATGAAATGCTGGGGAGTATGTGGAGCAACTGGAATTCACATACATTGCTGGTGGGCGTAAG
ATGGTACAACACCGGAAAACCATATGGCCTTACGATGTTAAACAAGCATCTATCTGTGACCCAGCAGTTC
CATTCCGATGGCATCCCCAGGAGAAACAGAAACACATGTCCATGAAAAGACATATGTATGCATGTTAACT
ACAGCTTTATTCATGACAGCCCCAAACTGGAAGCAGCCCAAATGCACATCAAGAGGAGAATGGCTAAACA
GACTATGGCATATTCATACAATTCAGACTACTTCTGAGCAATAAAAACAGAATGATCTACTCGCACACAA
TAACGTGGCTTAATCTTTAAGAAAATATGTTGAGCCCAAGAAGCCAGACACAAAAGAAAACATACTTTAT
GTTCCCTTTACACAAAATTCAAGAATAAGCAAAACTAATCTCTCTTGTCAAAATCAAAACATTGGTTGCC
AGGGTGGGGTGGAGGGAATGATTGTTAAGAATTGGAAGGGGACTTTTTGGCGTGATAGAAATGTGTCTTC
ACTGGGGTGGTGGTCACATGGATTTGCACATTTACCAAAACCTCATATACTTAAAGCCCATGCACTCCAT
GCGTTATAGATTTTACCTCAATTTTTAAAAAAGCTTCTGTGCCCCTTGGGGGCATAGATAGCTGTAGCTC
TTTGGCCCTCGAAATGCGAGCACAGTTTTACACTTTTGTTGTTATTTTAAAAACCATGCTGAGCCAGGCT
CAGTGGCTCATGACTATAACCCCAGCACTTTGGGAGGCCGAGGCAGGTGGATCACTTAAGCTAAGGAGTT
CAAGACCAGCCTGGCCAACATGGTGAAACCCCATCTCCACTAAAAATGCAAAAATTAGCCAGGTGTGGTG
GCATGTGCCTGTAATCCCGGCTACTCAGGAGGCTGAGGCAGGAGAATCATTTGGACCCGGGAGGTGGAGG
TTGCAGTGAGCTGAGATTGTGCCTCTGCACTCCAGCCTGCGCAACAGAAGGAGACTCCATCTCAATAAAA
AAACAAAAACAAGCCGGGCATGGTGGCTCACTGTAATCCCAGTACTTTGGGAGGCCGAGGCGGCCAGATC
ACCTGAGGTCGGGAGTTCGAGACCAGCCTGACCAACATGGAGAAACTCTGTCTCTACTAAAAAGTACAAA
ATTAGTAGTGCTTGGTGGCGCATGCCTGTAATCCCAGATACTTGGGAGACAGGGAGGAGAATCGCTTGAA
CCCGGGAGCCAGAGGTTGCGGTGAGCTGAGATCGCGCCATTGCACTCCAGCCTGGGCAGCAAGAGCAAAA
CTCCGTTTCAAAAAAAAAAAAAAAAATGCTGAAATACCTAACATCTCACAACGGCTTTGGCAAGTCTTTC
CAAAACAGAAGATTTATTTGTATAATTGGGTTCATGTGAAGTAGAACTCATAAAGACATCAAGAGCATAA
ATTTTTAAGTCCCAGAAGGTGACTTTAAAAAGGTTAATTATTCTGGAAAATATGAGGTTATTGGGAGCTT
ATTTTTCAAAAAATAAATGTTTAAGAAAGTGTTCTGACCATGTTAGTACTGGTGGGTCTTTTTTGGGCAG
AGGACTCAGAGTTTTGTATTTTCCTGACCCCGTTTCAATCTTAATGTTCATGCTCTTTGGATTTTCCATC
CTGTGGCAGCTGCGGTTTGCACTCAAGACCTACTTTCCTTACACTTCTCCATCTCTTGCCATGGTGCTGC
TGCAAGACCCTCAAGGTGAGTTAGGGTTGACTTTGCCCACATCAGAATGATTTCCTGGGAAGAGCATTGT
CAAATTATGACCTCCTCATCAGGCCCTTATCTACAGCATGAGGGAGACAGAACTGGTTATTTCTAAGATC
CCTTTAAGCCCCCAGGATCCCACATTCTGTGGATTTTGATATGAGAGTCTGCTTGGCTTCCCAGTTGTGA
TCACTTACCAAAAGAAGAAAACAAACAAAAAAACAATGGCCTCTGGTAGGTGTTGCTAGTCTGCCTCCAG
ATAACACAGGCTCTTCTGGTGCCAACCAGATCCACCAGATGCCAGGAACAGGCTAAGACTTGGCTGAGTC
AGAATAAAACAGTACATTCGTTTGCTTTTCATTCTTTCTACTCTGTGCAGCCTTGTTGCAGAAGAAAAGA
CAAAATGATGGATGAAGCTGAGGAAATGCAGGGGAAGTTTCCCCTAACCAAGCTAAGGGAAGGATTTTGA
GAGAGTTCAGTAAGGTGCTTCATCCACAGCCTCTCCTGATATCCTTTGCCAGCCTTCCAAAGGCTATCCA
GCTGGGGAAGTGATGGTTCCAGCCTTCATTCGACTGGTGTTGATCCAGGAATGTGGCTGTGAGCTCACGC
TTAGGAGACATTTCGGAAATAGAGAATAAGGGCATCCTTTCCCTCTGAGGCCTGAAACCTGATGGAAGGC
AGTCCTGACATGGGCACAGACTGCGCCAGCCAGAAAGGGGCCTTATTGGAGCATGGGAGGAGAGGAGACA
GAAGGGAACGAGCCAGTCACATTTCCTTTTTATGCCATTTTGGGCCTGCCTTTTTTCTGAGACTTGAGTG
AGTAGATCTGTTTGAGAAATGCAAATGATTCAAAGTTTCAGAGCATTTTACAAAACCTAAATTCCTCTTC
CTTTTTCGAGAAGATGTAGGTTCTTGCCCAGGGTGATCCCCTAACACTGTCTTCTCAGATGCTGGAGCTA
GGGGTGGGATCTCTGGGTGACAGTTTTATGAAAACTCTTGGGAAAACTTTATTAACAAGACAGAAAAATG
AGGAATTCACTCTCCCAGGGTTCACCTTACGCTTAGGTCAGATCAGCAAAGCCTAAAGTCAGCCAACAGG
GGACGCTGAGTGAGCTCCAGAAAATGTACTGGAGTTGTTAGTGTCTCAGTTTGGAAATAGTAAATAAACG
GTGAGGTGGTGCATGGGCATGGGCCCAACCCTTTACCTTTCCGTTTGTGTAACTTGTAGATTTATGTCCA
AATATAAATAGGTGGCTTCGGGCAGAAGTACAAGAACCTCTTGTGCTAGTTCCTAGAAGAGCTGTCTGTA
TATTACTTTGGGAAACAGTGTTATGGTCACAGATTGAAGAAGTGTTTTTTAGTGGCATGATCTTCTGTTT
ATCAGAAATTTATTTCATACCCATTGTGAAGGCCGGGAGAGGGGTGTAAAAGAGGATCACTTACGAGCCC
TGACTGTGTAGACACTACAAGTGGAGGAAATGCGAAATAAACATCTAAATAGCTAGTAAAAGGAAGAAAC
GAAAAATACCTTCAAAAGGCTGTGCACCAAGGGCCGCGGGAGTTCAGAGGAGGGAGGCGCTGGCCTTGTG
GAGTAGCAGCAGTTTGTCCATCACTCGCCTTACCTTCCTTGTGTGTTCGATTATAGCATCTATCACTATG
TGAAAACTTTTGCAGATGCTGCACAGAACTGCAGATACGTGCTGCTCAGCTGTATTTAAAACCACAGGAT
GACTGTGAAACCAGAGCAGCTCACTCAAGAAAAGACATTTGTTTGCATAATGCTCACTTATTTTCTTTCC
TGGCCTTTTTTTCCACTGTAAACCTGAAATCATATTGTGAATTCATCACAGCCCTAGGAAAGCAGTCAGG
CAGTCTATTCCTAATTCCTTATTGGCTGCTTTACTACAGTCTCCGTCTATTTTGAAATCAACACATTAGT
GATGGAGGAGTGTGTAGGTAGAAAAGAAGAAACCTAGGGCATCCGCAACCCGGCATTTCCCAGTCTCAGC
GGGGGCTGCTTATGCAATCTCTAGCTACTTCCATGTACCTGAATTTAAAATTACTTGAGGGCAAGCGTGG
TGATTAATGCCTGTAATTCCAGCACTTTGGGAGGCCAAGGCGGGAGGATCACTTGAGCCCAGGAATTTGA
GACAAGTCTGAGAAACATAGTGAAACCTCATCTCTACAAAAAAATTTTAAAAATAGCTGGGCATGGTGGT
GCACCCCTGTAGTCCCAGCTACTGGGGAGGCTGAGGTAGGAAGATCGCTTGAGCCCAGGAGGTTGGGGCT
GCAGTGAACCAGGATCGTACCACTGCACTCCAGCCTCTGACCAAGCAAGATCGTGTCTTAAAACCCCACA
AAACTAAAATTAAAAGATTAGATTCTTTGATGTTCAACTCTGTGTGACATGGACAGATCTTACTTCCCCA
GGCTTAAGAGATAGGTACTGGCTGGGAAAAGCATGAGAACAGTATTGGGTTCCGTGAACCAGAAGTAAAG
GGCGTCTCCCAAAGACTCTTCAGGTCATCCCTGCAGGTGGTTCCTCATGGGCTTGACATTTCCTCAGTTG
CCCTCTGACGTATCTCTCTCCACCCGCAGATATCCCTCGGGGACACTGGCTCCAGACACTGAAGCATATT
TCTGAACTGCTCAGAGAAGCAGTTGAAGACCAGACTCATGGGTGAGCAAACACTGACCACTCCCAAATCT
ACTTCACACATGGTTTCCCTAGATCCTCATCTGCCCTTTGAGCAGGGCTGACAGAGGAAGGCATTACTGG
TCTGGGACCATCACCAAACAGGTGAGCACCATGGTGAAGTGAGAAGTGGAGGAAGCGCATGGCTGCACGG
ATGGCTTGGAGCAGGTCAGGATGAATAGGCATGCGCATGTATGCATGTGCCAATACCTGTATCGAAATGT
TTTCTACATTAACGTCTGGTTTTAGATTTTGAAATATACAGGCATAACAATAAAAAGAAGAGGCCTTGGA
AAAAGCAGCACCCACCTGCTGGGAACCCCTCCCTGGCCGCACAGAAGGGAAAGGGCACTTGTTTGGGTCC
TGTGGTCTTCCCTCTACCTCTCTAGGGAAGGAGATTTGAAACTCCTACACAGTAGGGTGGAATGTTAAAT
GCTTGCTTTTCCTTCTCTCTAATAAATCTCTCTAAAATGAGAAAATCTATTCTAATATAAATTCTGGGTT
GGTTGGTTTGTTTTAAATTTTTAGAGATAAGGTCTTGCTCTGTCTTTCAGGCTGGAGTGCAGTGGAATGA
TCACATCTCACTGTAGCCTCAAATTCCTGCGCTTAGGTGATCCTCCCACTCAGACTCCTGAGTAGCTGGG
ACTACAGGCGTGTACCACCACACCTAGCTAATGTTTAAATTTTTTTGTGGCCGGGCGCGGTGGCTCACAC
CTGTAATCCCAGCACTTGGGGAGGCTGAGGCAGGCAGATCACAAGGTCAGGAGATCGAGACCATCCTGGC
TAACACGGTGAAACACCATCTCTACTAAAAATACAAAAAATTAGCCGGGCGTGGTGGCGGGTGCCTGTAG
TCCCAGCTACTCGGGAGGCTGAGGCAGGAGAATGGCATGAACCCAGGAGGTGGAGCTTGCAGTGAGCCAA
GATCGTGCCACTGCACTCCAGCCTGGGTGACAGTAAGACTCCGTCTCCAAAAAAAAAAAAAAATTTTTTT
TTTTGTAGAGGTCTTGCTGTATTGCTTCAAATTCATGGGCTCAAGTAATCCTCCTGCCTTGGCCTCCCGT
CGTGCTGGGATTACAGGTGTGGACCGCTGTGCCTGGCTTGTTAATTTCTTAACAGTAGTGTTAGGTACCC
TCTTATGTGTGTGATTTTCTTGAAAGCATTCACATTTATGCCTTGCTAAATTCCTTGTATCATAACACTC
TTTGGCTTCCTCTTTCAGAGTATAATTTTATTCATTGTCCCTGCTAGCCTACTAAGACTATCTTATTTGT
TCCTTTTGCCACTTTTCACTCCTGGAATTCTCTGTAGTCTCACACCGAGTTGACACAGACAATGGGTGAC
ACACCTATGTCCAGCTCCGTGATTGATGGCTTGGGTGCCAGCTGCTCACTCCTTCCTCACCATGATTCTT
ACCGACAGAGACCAGACAAAAAAGGCCTGCAGCCAATGGAGGCTGCGGGGTTGGCCACATGAATAGCCTG
CAGTGGCCAGTCGGCCCCCCACACACCCTCCCTGGTTGTCCTGGTGTTACACAGAGCTGGACCTTGAAGA
ATCTTAACTCGGGGACAGGACGTAGATGCAGGTGGCATAAACAGCCCACCCTGTGATTGAGACATGTTGT
GCAGTGGGTGCCCTGGGTGTTTTTCTCTTCTCTCTACCCACCCTCTCCACCATCACTCAGAATGTGTGAT
GTCAAATGCGTGAAGACTGGAATAAAACAGCAACCAGAAAGGGCGCATGTGGTGCTGCAGAACAGGACTT
AGACCTCGTGGGGCTCCCACTTCAATCCAGACCTTGGAGCCATGAGATACCTTTCAGGTGGCCACATGGG
ACTGCTTGTGCATTCAGCTACTGGTGGAAGACAGAGGGAAGGACCAGACCGGTCTCTGATCCTGATCCAG
TCACGCTGCCAACCTGGGAGCTGGGAGCATGAGGGAGCCTCACTGCTCTGTGCATCTGTCTACCCTGACA
TGGTGCCTGCAGGGAAGAGGCATGGCAGAGTCCCCTCCTCACTCTGGTACAGCCTAACCCAGCCCACAGC
CAACGTGGCCTCCAGAGCATCAGCTCTGACCATGTCCTGGGAAAGAGGGACATGTCTCTGTTGCTCAAAT
GTAGAACATTTAACCACTTGTTCTCAGGGAGGACTGGGGCAGGAGGACAGCAGTGTCCAGGGAGGCGAGG
ACAGTGCTTAGAGCTTGGGAATTCCATGGGCCAGGCAGAGGAACATGCAGGAAGATCCAACAAAGGGGCC
AGGAACACTCCTGTTCCAGCCACTGCCCCAGTCCAGAAGGAATGTTCTCATCTAATGTGATCTCAGGCTG
GTCTGTTCCATCCTCTCCAAGGGAGATGTTCACCTGCTGAGACCGGCACTGCACACAATTAGTAGGCCCT
TATCTCAAGCAAATTCTACCCTTAAAACAAGTATTGCAACTCCCATTTGCCAGTGTGGCGAGGGGATAGC
TTCACAAACACTTCAACTTTCTGGCATTGATGCACCCATGATGACAGCAGCTCATTAAAAATTCGTAGTT
GCTGAAATATCCACCTGAACTTTGCGTCTTGTCAGAAGGGAAATAGCCCATGTTTCTGCCTGCGTCCTAC
CCTCCACCTCTTTCTCCCCATCTCAGAAGGCAGGGAGGTGTGCCGCCACATCCTGCCACATTCCTGTTGG
TCATGGCCGCAGTCTTCCTTCTTCAGTGCCTGTCCTCACTGAACGTACCTGGATTAAAGACTCCGATGTC
CAAAGGCCTCAGGCCTCACGGGGGTCCTGAGAGGCACTCTTTCGATGTCCTGTGATGGTGCAGAGTGGCG
TTTGGACAGGCTTTCTGAATCTAAATTGTATTCCTTGCAGGTTGCACTTCAGTGGATAAGTACAATTTAA
GCCAACCGTTGGCAAACGTTAAAGTTGCTTTAAAAACCCAAAGGAAGAAGAATTTAGGTTGTCAACTGCC
ATGTGTTCTGCCTCTGTTCCAGGTCCTGCGGAGGTCCCTTTGAGAGCTGGTTCCTGTTCATTCACTTCGG
AGGATGGGCTGAGATGGTGGCAGAGCAATTACTGATGTCGGCAGCCGAACCCCCCACGGCCCTGCTGTGG
CTCTTGGCCTTCTACTACGGCCCCCGTGATGGGAGGCAGCAGAGAGCACAGACTATGGTGGGTAGCAGGC
CCCACTGCATGTGTTTGGGGTGGGCTCTGGGGGGCTGAGAGGAGCTTGCAGAGAGGGGCTCTGTTGTGGG
CTTGGCTTCCATGACACCTGAGCTCTGCCCACAGATGGCATATGTCATGGCAACCTGCAGGCATGGTGAT
CTCCAGCCTTGTGGTCAGAGGCGTTCTCCTGTCCCCACCGAGGTGGCCAGAGACGAGACGCTGCCAGCCC
ATTCTCCTGCAAAACGAAGACCCTTCTTCCCCAAGGTCATTTGATCTGTTATTGCTCAGCTGCCATATCT
GCCCCAGGGTCGCGTCCTCCAAGGCGTGCCCTGCAGAGGCGGGCTGTTCTGAGGGGTGTGCCGGCAGCAG
AGCCTGGCCACAGCCAGGGCCCTGTCTCGCTCCCCAGCAGCCCGGCCAGGGCTTGCTTCCATCTTGACTG
TCTCCTTTCTTGGATCAGTCAACAGATCCTCACATTACTGGCTTTATTTACATCTCTTACTAAAGTAATC
AGATGATATTAACCACTTTGCTTCTGAAAAAGAAGTTGGTCATTTCCTAAATAACAGAGCAGCTAAGTAT
GGGACTGGATAACTGTCTGTGGCAAAGACAGTTGTTATTTTATGTGGAATATAATAATTACAGGAAGCAA
TGCTTTGATTAAAGGCATTGGCACCTCCCTTGCAAGCATTGATTTGCTAGGTTCTTAAAACTTCAACTGC
CAGTGCTCTATGCTGATCTTAGTAGTTAAAGAGGACAAGGCTCATTTTGTATAGAGCCCTGCTTCTCTTG
GCTTTGGCATTTTCTGTTGTTTGAAAACACACAGACTAATAAAGATGTCTGCGTATTTTGTTTAAAGGCT
TGCATGGCATAGGAAAGAAAAATAGTTTTAGATTTTAAAAAGTTGAAATTTGGTGATAATTTATTTTGGA
TCAGAAATAAAGCTTTGTGAAAAAATTATAACCCACGTATGTATTTGAGGATTGCTGTCTGATTAAAAAG
AAAGCTTTTTATTTCTTCCCCTTTAAAAGATCACGTTAAGCTAAGGAAAATCCTGGTTGATGTTTTTAAC
AGTAATAAGATGTACGGGGCACATATGGTTTTTAAAGCCCTTTTATATTCTGTCTTATTTGATTCTGAAT
AAGCTAAGTTGGTAGATATGTGTTCAATTCCATCACAGTCAAAAGGTTACTGACAGAGAATCACTTTTGA
AGTATAAAGAAATAAATTATTCAATAACACTTAATTTCTAGAAAGTTCTACATTGGCACATCTTCTACTA
GCTAACTTGGTCCTTTTTTATGAAAAAATAAAACCCTTGCGTAGTTCTCCCTCAGGGGATGCCTAGGATT
TTGGATGAGAACGTATTGGCTCAATGTGAGTGGGGCAGTGGCAGGCATCCATTTCCCTTCCCCCCATTCT
GTCACAGGTGCCCATCTGCCTGGCAGTTCAATCCAGTGCTCATGTTGGTGACTCCAGAGCCCCTTCCTTG
CTGGTGCCTGCCTGAGGCATTGGTGTATGTGGCGTCCTGGGAAGGGGATTTTAGTTTAATGAATGATACG
TACCTCTTGTTTTCCTGGGTTTTGTGAGCTTTAATCCCTTGATGGTCTGTTGGGAGGCTTGAGAGACAAA
CTGTGAACTGTGTTAGAAAGCATGATTCGTATGTGATTGAATTGAAATTAGCAGTATCTCTGCATTTGTG
CATTTACTTAAAAAGGCACAATTAAATCAGCTGGTGGCGTATCCCCGCATCGTGTGCTGGGCCTGGCGTC
ACTGCCTCTGTGGATGTCCTTGAGGTGAAGAACATGGGTGCAAAGACCAAGCGTGTCCCCGCCCAGGATG
TGTCCTGCCTGTGAGAAGGCCTGTCCCACTCATGGGCAAGCCACCCTTTCCACCAGCAGCCAGAGTTCAT
CTCCTTTTGCCAACAACTGAATCAGATCACAACAAAATAAAAAAAAGTCGTTTAGATACCCTTAAGAATC
AGTAAGGTACAATGATGCCTAGAGAAAAAAAGGAAAAAAAAAAATCAGGCAAGGGTTCATACCCTTTAAC
CCTGTGGTTCCACTTCTAGGAATTACCCTGGGGAATAGAGATCCATGCAAAGGCTTATGTATAAGAATAT
TCAGCACAGCATTGTTCATAAATACAACAATGGGAAATGGCCTGAACTTTCAATAGTAAGGCTACTGGCC
AAATAAAGTGTGGTATATCCTAACCATGGAATAGCATTCTGTTACATACCATGAAAAATCATGCTCTTGG
ACGGGTGTGGTGGTTCATGCCTGTAATCTCAGCACTTTGGAAGGCCGAGGTGGCCGTATCACTTGAATCC
ATGAGTTTGAGACCAGCCTGGGCAACATAGACCCCTGTCTCTACAAAAAAATAAATAAAGAAATTAGCCA
GGTATGGTAATGTGTGCCTGTAGTCCCAGCTACTCGGGAGGCTGAGATAGATGGAAGGATCGCTTGAGCT
CAGGAGGCAGAGGTTGCAGTGAGTAGAGATTGCACCACTGCATTCCAGCCTGGGTGACAGAGCAAGGCTC
TGTCTCCAAAAAAAAAAAAAGGTCTTGAAGAGCATGTTCTATAGATACAGTGAACTGAACTGCTTATAGT
ACATTATGGAGAGAAAAATGAAAGATACAAGGCATTATGTGTAGTGAGTGGTATGGTCTCAGTGCGTAGA
AAAAAGACAAGAAGTATATAAACCGAATTATTAACAGTGAGTCTTTTTGGATGGTAGAGTTATAGATCAT
TTTAATGGTTCTTCTTTTTCTCTTTTCAAATTTTCCATAGTAAGCATATATGTCTTTATAATGCAGGATA
AAAAATACTTTAAAAAGAAAAGAAAGCTTTTATCTTTCTCCTGAAAAACTTAGGACATGTTTAAAGTTAG
TTATCGGTGTTCAAGTGGAAGCCCATGAGCATGGACGGCCTCTGAAGGGAAAGACAGTCTGGGCCAGACA
TAAAGGAAGATAGACCCCATCTTAGGGATTATTGGACAAGGGCAGAAAGTCGGTTGTGGGTCAAGAGAAA
AAACAGATATAAAGGCATAGCCTCCTAAACCCTGACATTTTTGCAGACCCAGTTAGATGGCAGAATGGCC
TTGGGCTCCCGTAGACACCATCAGACCCGGCAGGCTGGGCCGGCTGAGGGGCGAGGTGGGGAGAGTGAGG
CCTGCCCTGCTGGAGCGCTGCCGCTCCCCGTGGAAGAGCCCGATGGTGCAGTTTCTCACGGTCTTGTATT
GTGGTCAGAGGAGCTCACACAACACACCCAGTGTACTGGAGTTTATGACAGACTTCATTGAATGAGGCCT
TGACTTCCAGGGAAAAAAAACGCCTCACACAAAATCCATAAAATCGGTCATGGGAATTCTCAGAATAGGC
CTAAGTATTCTTTCAGAAGTGATGGAACATAGCTGTTCACGAGTTCCATGTGGCATTTGGAGCTATGGGC
TTTCGGTTTTTGCTTTGCTTTGTTTTCCAAGTCACGACTGGGTGTACCATTCTTGTAGCAAGCAGAAACA
TCTCAGCATGGAATGAGTATTTTTTATGCAAAAGCAACCTGCAGACTTATAATTACCATTATTATGTGGG
TTCCACAGACTTGTGGCCTCTGCTTGAGCATTAAACACGTATGAACAGATTGTTCCGGCATCTAAAACAA
ATGTGTTCTTGTGTGTGCATGCATGTGTGTATGACCGTGCTTGTGTACACGTGCGTGCATGTGTGTGTGT
TCCCCCCGACCCCCCGCCCTGACCCCCAATATAGCAAGAAGCAAAGGCAAGCCTACATTCGTATGGGAAC
ACTGGCTCGGCGGAGGTCTGACTGCGTGGGGCTTTAATTGATTTGTGGCTGTGATAGGCGAGTCTTCAAG
GAGCCTTTTGTTCTTTTTCCCACAATATAAAAATCTGACTACATTCTGTTGAGAACTTTTCTCTGTTTCC
TTTTATACAAAAAAAGGTCAGTTCCTGGCATGGTGCCCTGACTGCTTGGTGAGCAAGACGAAAGAAAGTC
AAGAAATTTCCATTCCGCCCATTTGGGTTTGTGTGAAATCGAGTGTAAATTCGGCCGCTGCCTAGCTCCC
TCTCTCAGGGGCCAGTGCTTACCCGTTTCTGGGGGTTGTTTCAAAGCAAATAAATAAATCCTAGAAGTAT
GTCTGTCCTGCCTCTTCCTAACCTCTCCCCTGTGAAATACTATTGCCCAGGTCCAGGTGAAGGCCGTGCT
GGGCCACCTCCTGGCAATGTCCAGAAGCAGCAGCCTCTCAGCCCAGGACCTGCAGACGGTAGCAGGACAG
GGCACAGACACAGACCTCAGAGCTCCTGCACAACAGCTGATCAGGCACCTTCTCCTCAACTTCCTGCTCT
GGGCTCCTGGAGGCCACACGATCGCCTGGGATGTCATCACCCTGGTAAGTCTCCCTGTGGTCCAGCATCC
TAGTACTCATTTCTGCTCCAGGCGAGAGGTGGGTTACCTGTCCGAGGGTCTGGCCTCTGCACAGTGTGTG
TACCAGGATGCTGCCCAGCACAGATAAATGGTCAAGGAGGTTGACAGCTGGGATGGACCCCAGAGCCTGC
CCCTTCCAGCACCTTTATGTCATAGAAGAGAAAACAGAGGCTCAGAGACAGGAGATGCCATCTCCTGGGC
CCACGGCTTGCTGCCAAAGCCTGCGGTAGAAGCCAGGTCTCCTGTCCCCCTACGTACATTTCTTTCCATC
GCAGTGTGCTGCTCTCCTCCTCCTCATGGAAAGAAGTAAGGCCACGATGAGAATGGGCTCGCAAAGCAAC
CACAATGCTATTCACTGCAACAGTGCAAAGAATTCTGTTAATCTTTAAAAAAATTGGTTTATTACAAAGA
AATCCTTCCTGAAGCCCACTTGACTTGAGGGGTAAAAAACGGTCCAAGTCAAAAGTGAGCACTCTTTGTG
ACATTAGATTTGGCAATGATTTCTTGGATATGATACCAAAGGCACAGCAATGAAAGAAAACAATAGATAA
ATTGGACTTTATGAAAATTAAAAGCTTTTGTGCATCAAATGATACTGTCAAGAAAGTGAAAACACAATTC
ACAGAATGGGAGAAAAATATCTGGAAATAATATATATGATTAGAGTTTAGTATTCAGAATACATAAGGAA
TTCTTATAACTCAACAACAAAAAGATAAACAACCCAATTCAAAAATGGGCAAAGGACTTGAACAGACATT
TCTCCAAAGAAGATGTACAAATGGCCAGTAGGCATGTGGGGAGATGCTCACAGAAAGGGCATGAGTGGTC
AGGGAAAGGCAAAGTGCAGCGAGATGCACTGCACAGCCATCAGGATGGCTGTCAGCAAGGAGAGAAAAGA
AACGCCCGTGAGGATGCGTGGGAACCGGAACACTCATATGCTTCCCGGTGGGGACATTAACGTCGTGCAG
CTACTGTGGAGAAGAATGGGGTGGTTCCTCAAACAGCCAAACTTGGAATTACCATGTGGCAGGCAGTTCT
CCTAGGTCTATAACCCAGAGAGCCGAAAGCAGGGGCTGGAGGGCCATCTGGCCCAGTGCTCAGGGCAGCA
CTATTCTCAGCAGCCAGCAGGCAGAGATGGCCTAAACGCCTGTAGAGGGACAAGCAATGCACCGTGTGAT
CTGTACCCACAAGAGAGTACTGTTCAGCCTGAAGATGGAAAGAGGTTCTGACAGTGCTACAACATGAATG
AACCTGGAAGACACTGTGCTGTTGAAATAAGCCAGAAACGAGGATGTTGTATGAGTCACTTAGGTGAGGC
ACCTGGAAGAGTCAAGTCCAGGGAGAGGAAGTGCTAGAGTTTCCCAAGGCAGAGGGTGGGGAGTCAGTGT
GGGGATCATGGAAAGGGGCAGGAGACGGCCAGTGCTGATGGCTGTGCAACACTGTAAACATACTGCATGC
CACCAAATTACACAAATGGGTAAAACAGCACATTTTATGTATATTTTACCACAATTTAAAAAAATACTCA
TCTCAGAAAAAGCACCAGCATGCTGGCTCTGGGAGTTTTGTTTTATTTTTTATTTTATTTGTACTTTTGT
TGACATATACATACAGAAAATACACAGCTTGATGAATTTCACAAACCAACAACACCTGTGCAGCCAGCAG
CCAACTCGAGAGGAATATTCCCAGTCCCCAGAGGCCCCTCCTGCTCCCGTCCAGGCCGGGCCTGACTGAA
ACGTGAATGTCCATGTACAGAAAGTCCCCGTAACATGACCCACCTGCCAAGAACCAGGGCCAGGAGATTG
TCTCCATCCTGGTAGGACAGTCCTGTGGAGTGGGCCCAGGCTGTGCCTCCCAGGTGCTGGTGTCTCAGCT
AAGACGAATCCGAGTCATGAGGGTGGCCCAGGGCTAAAGACGACTGGCTGCCACATGCCAGGTGCCACCT
CTTTGGGCCCGGCCCCAGCACAGCGAGAATGCTTTCGAGAATCTATTGTGTACTCTTATAAAATGCACAC
CTGCAACAATGCGGCCCAACCTAGAGGGCTTTACACTGAGCGAAATAAGCCAGACGCGAGAGACAAATGC
ATGCTCTCACCTATGGGTGGGATCTAAAAAGTCGAACTCACAGAAACAGTGGGAAGGTGCTTATGGGGGG
CTGGGGAGGGGCAGTGGGGAGATGGTCAGCCCTGCCCTGTGACAAGGACAATTCCAGCTCAGGCACTTCC
TCCCCAACCCTGTGGCCCGTGTGAGTGGAGAAGGCTTAGCAGCAGGGGTCAGCGTGGGAGTTTGGTGACT
CCAGCCCTGCCGACAGGGTGTGAGAGAAAATCACCCAGAGCAAGCACCCTAGAGACACCCAGGTCTCTGC
TTCAGTTATCCTGATGCCATGTTTCCATCTACTCGGAGGAAACTTTCAGTCCACATTCAAAGCAGATCGC
CCTCAAGACTGGGCCCAGAGACATTGCTGGGAGTAAGCCAGATGCTGCAAGGGCCAGCCTGCATCCTGTG
TGGGGCAGTCCCTCCTGACACGTGGTTCAGCCCCTGCTGCGGATCCACACGGGAACCCTCAGGAGCACAG
AGGGCCCCAGTGCTTTGTCATCACATCCCCCAAGTCAGTTCCGCCACAGAGAAGACACACACAGCAGAAG
CCCATTCCCAGAGCAGACGCCAGTGTGGCGCTCATGCACTGACTCTGTCAGGGACGGATGCTGGCCATCT
GGGCACCCATCGTGGTGAAAGCACCTCCTGGGGGCTGTCCTTTCGGATGTTTTGAAACCATCTTTATTGA
CACGTCAGTCTTCCTAATGAGAAGCCTTCGCTGCGGTGGCCGCGTGGCAGGGAGGGCCTCTGGGGAAGGA
TCCCTCCCACCACCCCTGCCTCTCCTGGCCTTTGTCAGTTCCTCTGCCGACAGCACCTTGGAAAGAGCCG
GGTCTGCTTGGCCCACGAGCAGGTGGTGGCCAGATGGTTGCTTTGGTGGGACGGCCTTTCCCTCCTACAG
CTTGGGTCTCTCCACCCTCAGGACCTAGCACGAGCGCAGCCACCTCCCAGCTGGTCCCAGGCTCTGCCCT
CTATCTTGGCATCCAGATTTGGAGAGCAGTGCCAGCTCAAGGCAAAGGCCACTATCTCTCTTCAGAGCCC
TTGCACCCTCTTGGAGATGGCTTGAGCGAGTGGCTCCATCTGACCTAAAGGACGCACTGGTCCTGCAGCC
CCATCTGCCCACAGCCTGCCTCCTGTGTGGCCTCTGCAGTGAGCTCTGCGCAGTGGGCTCTTCTCCAGGC
ACCTGGAGGCCCACTCCCCGGCTGTCCCGGAATCCTCTTCTCTGACCTCGGCCCTCCGATTTTCCCCAGA
ACGCCATCCATGAGGCCCTGCCCAACGTCCCTGCGGTTCTCCTGCCTCTCTGGCTCCTGGCCTCTCGCCA
GGCACTCCTCTGCCTTGCCCCAGCCGTCCACTTGCTCCCTCACTTGTGCCTGCAGGAGGCCGGCACAGAG
CAGAGGACTGCATGGAGTCCACCCCTGGGGCTCCACCTCCTGTCCTGTCCTGGATTCACACTTCTCCTTC
TCCTGCCTCCTGCTGCTGGGCCACAGCAACAACAGCAAAAGTTAGCTAGATTCCGAGATGCAGAGTTCAA
TTCTGTAGTCGTTGAACTCAAAATGAATTGAACCCACTCTTGGTGCACCAGCCAAGGCCAGCAGGGAGGC
AGGTTTTCCAGGCGGCTGCAGGGCTGCGAATTTTTGCATGTGACTAGAGAGCTGGGAAGGGGCCCACCCA
GGAGTGTTGGTGTGTAAGGAAGATGCAAGGTTGGATTTGTAATGACAACTCGGCCTTTCTTCAGACATGG
CCAAGCCCATCAGCATGGGCATGGCCAGCCCCTGAGCGGGGGACAGTCCTCCCGCTCAGGGTCTGGGTGG
TATATTAACTGAAAGGGGTCCACAGCAGGCTCACATTGTTTCCTTGATGAAGGACAGGCTGCATCTACCG
AAGGCCATCAGCACTATGTGCCTGTCATTTCTGAGTAAAAACCAAGTGATAGCAATACAGACGCCCAGGG
ACACACTGGCACGTGCCTGGGCTACAGCCGGAGCCCCCGTGCTGGCTGCACTGACCCTGGCGGGCTCGTT
GCAGGCCGGCTGCAGGCCCTGTGTGCTGCCAGGGGGGGCCGAGGAGAGCGAACGTCATGGTCCACACCAC
GTCGTTTACTCTTAGCTCTGCGTCCCCACGCTGCCATTCACACAGTGCGCATGCGACAAGAGGCTCCCTT
TAGTGGTGGTCAGGAGGTGGAAGGCCCAGTTCTGAAGGGTGTCCTGGGCTGTCGGACCTGAGGCAGTAAA
ACCAGAATAGTCCTGGGAAAACCAAGGCAGTTGTTCACCCTCCCAGAACGGCCCAACCCTTCCAGAGGAA
GCTGACCTTGGGCTGCTCTGGCTAATTAAACATTTGCGGGTTCTGTATAAACTGACACACACTAAGTGAA
TATATCGGTTACTCTTTCAAAAACTTAAGCCTTCTTAATACTGCTTGTGCTTAAAGAACCATAAATTCCC
CACACAGCTAAATTGTAAGATTCTCACCCTTCCCTTTTTTCTTAACCGAAGTTTTTAGGAAAATACTTAC
AGGCACCAACAAGTACGAATGCCTTGAGGCTGTGTCTCCTGCTCTCCCGCAAGAATCAGGTGTTAAAACC
TTAGAGGCCTATTAAAAAGCAAGTAGGTCCGGTTGCTAGCCCAGGGATGCCAGGGGCTGTTTCTGTGAAA
ACAGGTGGTATTTTCCAATTCTCATGTTCGTGTGAGCCTTTTGCAGAAAGTCAAGCCACAGCATGCTCAT
TTGCCTTTGTTCCCCACAGTGGATCATCCCACAACTTGTGACTGGCATTCTGAGAAGCACTTCTGATTGC
AAGCAGCGCTTGTTTGTGGCCTTTATGAGTCTAAGGCTGTTGGCAGCTGAGCTCAATGCGAGGAGGCTCG
TTAGGAAATGGCACCGCTCACTGTGTAGCCAGCCTTGGAGACAGACTTGCTATGCTAATCACCTTGCAAA
CTAGAGCCCCTGATGCTTTACTTTGGAACTGATGGAAATGCTGGATAGGGCTTCTTTCAGGGACTGGGTG
GTTATGGTCCGTCCCTGGACAAAGGACAAATCTGTCTGGAAAGTGTTTTAATTTGCCTTCTCTTCTGTCC
TGATTGCAGATGGCTCACACTGCTGAGATAACTCACGAGATCATTGGCTTTCTTGACCAGACCTTGTACA
GATGGAATCGTCTTGGCATTGAAAGCCCTAGATCAGAAAAACTGGCCCGAGAGCTCCTTAAAGAGCTGCG
AACTCAAGTCTAGAAGGCACGCAGGCCGTGTGGGTGCCCGGCGTGAGGGATCAGGCTCGCCAGGGCCACA
GGACAGGTGATGACCTGTGGCCACGCATTTGTGGAGTAAGTGCCCTCGCTGGGCTGTGAGAATGAGCTGT
ACACATCTTGGGACAATCTGCTAGTATCTATTTTACAAAATGCAGAGCCAGGTCCCTCAGCCCAGACTCA
GTCAGACATGTTCACTAATGACTCAAGTGAGCCTTCGGTACTCCTGGTGCCCGCCCGGCCAGACCGTCAG
CTTGATAATTACTAAAGCAAAGGCCTGGGTGGGAGAACAGGTTTCTAGTTTTTACCCAAGTCAAGCTGCA
CATCTATTATTTAAAAATTCAAAGTCTTAGAACCAAGAATTTGGTCATGAACCATTAAAGAATTTAGAGA
GAACTTAGCTCTTTTTAGACTCTTTTTAGGAGTCAGGGATCTGGGATAAAGCCACACTGTCTTGCTGTAT
GGAGAAATTCTTCAAGGGGAGTCAGGGTCCCTCAGGCTTCCCTTGTGTCTCCCTGGACCTGCCTGACAGG
CCACAGGAGCAGACAGCACACCCAAGCCCGGGCCTCCGGCACACTCTTTCCACTCTGTATTTGCTAAATG
ATGCTAACTGCTACCAAAAGGCCCTTGGGACATCAGAGGAGCCGGCAGGCGAAGGTAGAGGATGTGTTCC
AGAAACATTAGAAGGCAGGATTAATTCAGTTAGTTAGTTCTCTTGTTAAATGGAAATGGGAATTGGAAAT
TCCTGATAAAGAATTGGCCTGGCTGGGTGCAGTGGCTCACACCTGTGATCCCAGCACTTTGGGAGGCCAA
GGCAGGGGGATTACTTCAGCCCAGGAGTTCCAGACTGCCTGGCTAACATGGCAATACCCTATCTCTACTA
AAAATACAAAAATTATCGGGGTGCAATGGCATGCATCTGTAATCCCAGCTATTCAAGAGGCTGAGGCATG
AGGATCTCTTGAACCCGGGAGGTGGGAGTTGTAGTGAGCCGAGATCATGACACTGCACTCCAGCCTGGGC
AACAGAGCGAGACCATCTCTTAAAAAAAGGCATTGTTAGTGTAATCTCAAGGTTAACATTTATTTCATGT
CAGTACAGGGTGCTTTTTCCTTTCAGGGACATTCTGGAATTGTATTGGTTGTACATTCTTTTGTGTCTAT
TCTGTTTGTCAAGTGAGTCAAGACTTGCTTTTGTCCATTTTGATTTGTGTGTATTAGTCTGAGTCTTGGC
TCCGTTTTGAGGTATGAGCAAAGTTTTGCTGGATTAGAAGTTAACCTTTAGGGAAATTCCTTATTTTGGT
ATGTGGCAATGCTAATAGATCCACTGAAGATCTGGAAAATTCCAGGAACTTTTCACCTGAGCCTTTCTTC
TGAGAAATGCTGCAGTCAGAAGGGTGTGCTGGTAAAGTATTTTGGTGGCAGCTGCCATCATGGTCATTGC
CTTCATATAACATGCTTCGTGCTCATGGTCATTGCCTTCATATAACATGCTTCGTGCCATCATGATCCTT
GCCTTCATATAACAAACATGCTTCGTCAGAGGTGTTGGGGTTGAAAAAGGAGCTGCATGCTTCACTGGAG
TTGAGGGCCTCTCTCCTGTTCTGACTTTAAGCCAGAACTTGTGGCTGGGCCATGGAAGCTGTGACTCCTC
TGTGGACATGGTGGCAGCAGGGAACCCCTAGAGAGAGGGGCCACTGGGACCAGGCCTCCTGTTGTGGAGG
GACTCCTGGGACAGTCCTCCACCCTGTCCTGTGGTCCTGTGTACAGGGTTGGCCTCTTCCTCCTCCCCTG
CCAGGCCTCTGCCCATGCCCCTTCCTTCCTTCTCCTGGGACTGGTGAAGCTAGGCATCTGGAAGACTTCT
TCCTAGCCTGGAAGCCCTGACCTCGGCCCATCTGCAGAATCTCCCAGTTCCTTCACAGCTGCCGAGTCCT
CTCACGGGTGCGGTGGAGGCGGCCTTGCCGGTGGTGCTTTCTGGGCAGCCAGGGGTTCCTGGGTGGGAGG
ACTGTCCCTCTGGGGACGTGGCACTGAAGTGCCTGCTGGCTTCATGTGGCCCTTTGCCCTTTCCCAGCCT
GAGAGATGCTCAAAGGTGGGGAGCTGGGGGAGCCACCCCTCGGCCATTCCCTCCACCTCCAAGACAGGTG
GCGGCCGGGCAGGCACTCTTAAGCCCACCTCCCCCTCTTGTTGCCTTCGATTTCGGCAAAGCCTGGGCAG
GTGCCACCGGGAAGGAATGGCATCCGAGATGCTGGGCGGGGACGCGGCGTGGCCGAGGGGGCCTTGACGG
CGTTGGCGGGGCCTGGGCACAGGGGCAGCCGCAGGGAGGCAGGGATGGCAAGGCGTGAAGCCACCCTGGA
AGGAACTGGACCAAGGTCTTCAGAGGTGCGACAGGGTCTGGAATCTGACCTTACTCTAGCAGGAGTTTTT
GTAGACTCTCCCTGATAGTTTAGTTTTTGATAAAGCATGCTGGTAAAACCACTACCCTCAGAGAGAGCCA
AAAATACAGAAGAGGCGGAGAGCGCCCCTCCAACCAGGCTGTTATTCCCCTGGACTCCGTGACATCTGTG
GAATTTTTTAGCTCTTTAAAATCTGTAATTTGTTGTCTATTTTTTCATTCTAAATAAAACTTCAGTTTGC
ACCTAATGTTGTATCCATTAAATATTACAATCCTTCTCTCAGGAAAAAGCTCCCCCGATGGCCCCAGACA
GTGGAAGCTTCGGGCCTCAGGAAACCTAGATGTGCCCTGGAAGAGACCGAGGATGGCTTAGCCTCTGGGC
CTGGGAACCTTGCTACTAAATACCTAAAAACTAAGGGTCCTGTCACCAAGGAGGAAGCAGGAGGACAAGG
GCAGGCATCTGGCAGGGCCTGCCAAGATCAGGTTACATGCGTTTCATATCTGTGTTTTCTCTAAGCCAGG
CTTGACCATAAGCCTCACTTGAATTGTTCTGTCTCTTCCCTGATCCCTGAAGCTGAAGGCCTCGTCCCAC
CAGCATAGCCCACCCACTCTTGCTCGCTCCGTTGCTTCACAGGCTGCTGTCCCCTCCTCCAGCCCCACAG
GCCCCTCTGGGAGGCATGAGTGATGCGGCCAAGGCGAGCAGCGCCGTGGCCACCCCCAGGACCCCCACTG
CAGTGGGGTTTCCAGCACTCCCCATGCTTCCGGCTATGGGACACTAGGGCCCCTCTCGGGGTCAGGAGGG
CGCCTGAGGGAGCAGACAGGAAATGCTAAGACACTCCTGAGGAGCCTCTCACTGGAGCAGCCATGGTTGG
CAGGGTTTCGTGGTTTGTTCTTCACCCTGTCCTGCCTGGTGTCTAAGTGGTGGGACAAGGTGCTAGGACA
GAAATGGGAGCTAGGACAGAAATGGGAGCTAGTCCAGGGGATGGGAGGAAGCCGAGTTTTCTGTTGAAGT
GGAGAAGTTACGAATGGGGTCTTGACAGGGAAGCCAGAGAAGAAGGGACCTTGGTGAGCCTGGCAGAGTG
AATCCACCCAAAATGCTGGGAAGTCAAAACCGAGCCTGGCCTTACGCTGCCCAGAGGACGGCGGATGCAC
TGGAGGATTTGCACAGTGGGAGCAAATGTGAGGAATGTTCTCCTTATTCAACAAGGAATCCAAAATTTTT
TTGCTTTCCAAGGAAGAAGAGAAACAGTTCCCCGTGCTTCTCCCCTCGCCTTTTTCTTCCTCTGTTTCCA
GCCCAGGACCACCTCCCCCAGCCCGGGGGCTCGGGTGCTAAAGTTCTTCCCTCTGCCAAATGTTGGGAAA
TCCGTAGATTTTGTCGAAAAATCCTAAGTCATTAATCATCGCTAAGCCATTTTACAACCAAAGAACAAAA
ATTAAGATACAGTAAGTACATAACATGTCTTCAAGCCTCCTCCTAAAATCCAGTTCCACAATTGGTTTAT
ACCATGGGTCCAAATCACACCCCTCAAGCCCAAACTGGTAATGGAGAGCTCATAGCAAAGGCCTTCCCTC
CCGCTCCTGTCATGGTGTCTTACTGCACTTGCTGTTCTGGGCAGGGAATTTGTTCACCCAGGACTTTTGG
TTGCAAGCAACAGAAACGTGCACTGAATGCTGAAAGTAAAGGGGAACTGATGAAAAAGGTATTGGGCTGA
TGAGGCCTGGTGTGAGCCGGCCAGCAGCAGTCCCAGCCTTGCTTCAGATGCTCTGGCAAGGCCAGTCGTG
CTGCCCAGCCCTTCCGGACCCCACTCCAGCCGGGAGAAAGGCCCTTGTGCCCACTCCTTTCCACACCCCT
GCCCAGGTGCATCAGATGGAGGAGCCTGGGTCAAAGGCCTGCCAGGGATGCTAGGAAATCACGCATCTGG
CTTCAGCTTCTTTAACGTTATTGGGTACCTTGGATACCACCAAAAGCATCACCCAACTACAGGAAGGGGC
CAAACACTGGGCAAATGGAGACAATAAAAATGTCTCCAGCTCATTACAGCATCTATACTTTAGCCTATGC
CAGAGCATTTTAGAAGGTTGCGTCCCATGAAACAGCCCAGCAGCCAGCACCAGAACAGGCAGAGGTGAGC
CTTTTCAGAAAGATGTTTTGTAGGCACCTAAATCTGGGTTTCTTAAATGCAGCACCATGAGCACTTTGGA
CCAGATGCCTCCCTGTTGTGGGGGTGTCCTGTGCACTGCAGGGTGT

### Examples

The following examples are provided for illustrative purposes only and are not intended to limit the scope of the claims provided herein.

### Example 1. General Methods

PEgRNA assembly: PEgRNA libraries (and ngRNAs) may be assembled by one of three methods: in the first method, pooled synthesized DNA oligos encoding the PEgRNA and flanking U6 expression plasmid homology regions may be cloned into U6 expression plasmids via Gibson cloning and sequencing of bacterial colonies via Sanger or Next-generation sequencing. In the second method, double-stranded linear DNA fragments encoding PEgRNA and homology sequences as above may be individually Gibson-cloned into U6 expression plasmids. In the third method, for each PEgRNA, separate oligos encoding a spacer, a gRNA scaffold, and PEgRNA extension (PBS and RTT) may be ligated, and then cloned into a U6 expression plasmid as described in Anzalone et al., Nature. 2019 December; 576(7785):149-157. Bacterial colonies carrying sequence-verified plasmids may be propagated in LB or TB. Plasmid DNA can be purified by minipreps for mammalian transfection.

Alternatively, PEgRNAs (and ngRNAs) may be chemically synthesized. The PEgRNAs and ngRNAs may be modified at the 5′ end and the 3′ end: the three 5′ most nucleotides may be modified to phosphorothioated 2′-O-methyl nucleotides. The three consecutive nucleotides that precede the 3′ most nucleotide (i.e. three consecutive nucleotides immediately 5′ of the last nucleotide at the 3′ end) may also be modified to phosphorothioated 2′-O-methyl nucleotides.

### HEK293T Cell Culture and Transfection:

Strategy 1: HEK293T cells may be propagated in DMEM with 10% FBS. Prior to transfection, cells may be seeded in 96-well plates and then transfected with Lipofectamine 2000 according to the manufacturer's directions with DNA encoding a prime editor fusion protein and PEgRNA (and with ngRNA for PE3 editing strategy). Three days after transfection, genomic DNA (gDNA) can be harvested in lysis buffer for high throughput sequencing and sequenced using MiSeq.

Strategy 2: Alternately, HEK293T cells may be propagated in DMEM with 10% FBS. Prior to transfection, cells may be seeded in 96-well plates and then transfected with Lipofectamine MessengerMax according to the manufacturer's directions with mRNA encoding a prime editor fusion protein and synthetic PEgRNAs with or without ngRNAs. Three days after transfection, gDNA can be harvested in lysis buffer for high throughput sequencing and sequenced using MiSeq.

Lentiviralproduction and cell line generation: Lentiviral transfer plasmids containing the IVS4+4 A->T mutation or the 322delG mutation with flanking sequences from the FANCC gene on each side, and an IRES-Puromycin selection cassette, are cloned behind an EF1α short promoter. HEK 293T cells may be transiently transfected with the transfer plasmids and packaging plasmids containing VSV glycoprotein and lentiviral gag/pol coding sequences. After transfection, lentiviral particles may be harvested from the cell media and concentrated. HEK 293T cells may be transduced using serial dilutions of the lentiviral particles described above. Cells generated at a dilution of MOI <1, as determined by survival following puromycin, may be selected for expansion. A resulting HEK293T cell line carrying the IVS4+4 A->T mutation or the 322delG mutation can then be used to examine PEgRNAs designed to correct the mutations.

Installation of IVS4+4A->T mutation or 322delG mutation in human FANCC gene by prime editing: Prime editing guide RNAs (PEgRNAs) may be designed to incorporate a IVS4+4A->T mutation or a 322delG mutation in a wildtype human FANCC gene in HEK293T cells. Briefly, plasmids encoding U6 promoter-driven pegRNA expression cassettes and prime editor fusion protein are co-transfected into wildtype HEK293T cells as described above. Seventy-two hours after transfection, gDNA is harvested for high throughput sequencing and may be analyzed using Miseq. Frequency of the installation can be used as a proxy to examine prime editing efficiency. The resulting HEK293T cell line that carries a 322delG or a IVS4+4A->T mutation can be used to test PEgRNAs designed to correct the mutations.

Generation of clonal cell lines containing mutations in the FANCC gene: wild type HEK293T cells may be transfected with installation PEgRNAs and polynucleotide encoding a prime editor protein as described above for incorporation of the 322delG or the IVS4+4 A->T mutation. Editing efficiency of mutation incorporation may be determined with Miseq. To generate a clonal cell line homozygous for a mutation, seventy-two hours after transfection, HEK293T cells may be detached from the growth surface and plated at a limiting dilution to generate clonal colonies. After seven days of growth, single colonies are mechanically detached from the dish and transferred into separate wells. Once grown to near-confluence, cells are split into two fractions: fraction #1 is propagated while gDNA is harvested from fraction #2 for sequencing with Miseq. Clones that exhibit 100% editing of the target site (i.e. homozygous for the mutation) can be used to further examine prime editing correction of the mutation.

### Example 2. Spacer Screen for IVS4+4A->T Mutation in FANCC

Single guide RNAs (sgRNAs) that contain a spacer associated with a NGG, NGA, or NG PAM within about 100 nucleotides flanking the IVS4+4A->T mutation were examined for Cas9-mediated cutting activity in wildtype HEK293T cells. Spacers of the tested sgRNAs include potential PEgRNA spacers, potential ngRNA spacers, and spacers that can be used in both PEgRNAs and ngRNAs.

Briefly, one day prior to transfection, wildtype HEK293T cells were plated in a 96-well plate at a density of 10,000 cells per well. Before transfecting, cells were approximately 60-70% confluent. The cells were transfected with MessengerMax transfection cocktail containing an mRNA encoding a Cas9 protein capable of recognizing a NGG, NGA, or NG PAM, and a sgRNA containing a test spacer associated with a corresponding PAM. Three days post transfection, gDNA was harvested with QuickExtract Solution (Lucigen) and sequenced by next generation sequencing. Sequences of the sgRNAs, tested spacers, and the results are summarized in Tables 63 and 64.

TABLE 63
sgRNA sequences used in spacer screen

| SEQ | | |
|---|---|---|
| ID | | |
| NO. | Description | Sequence |
| 3566 | sgRNA | GCACCTATAGATTACTATCCGTTTTAGAGCTAGAAATAGCAAGTTAAAATAAGGCTA |
| | | GTCCGTTATCAACTTGAAAAAGTGGCACCGAGTCGGTGC |
| 3567 | sgRNA | CTCTTTTCACTCAAGGTCTTGTTTTAGAGCTAGAAATAGCAAGTTAAAATAAGGCTA |
| | | GTCCGTTATCAACTTGAAAAAGTGGCACCGAGTCGGTGC |
| 3568 | sgRNA | GAAGTTGCTCTTTTCACTCAGTTTTAGAGCTAGAAATAGCAAGTTAAAATAAGGCTA |
| | | GTCCGTTATCAACTTGAAAAAGTGGCACCGAGTCGGTGC |
| 3569 | sgRNA | TCCTGGTTTGCTTAAAAATGGTTTTAGAGCTAGAAATAGCAAGTTAAAATAAGGCTA |
| | | GTCCGTTATCAACTTGAAAAAGTGGCACCGAGTCGGTGC |
| 3570 | sgRNA | GGTCTTGGGTATGCACCTATGTTTTAGAGCTAGAAATAGCAAGTTAAAATAAGGCTA |
| | | GTCCGTTATCAACTTGAAAAAGTGGCACCGAGTCGGTGC |
| 3571 | sgRNA | AGCATTAAACATTTCAAAAGGTTTTAGAGCTAGAAATAGCAAGTTAAAATAAGGCTA |
| | | GTCCGTTATCAACTTGAAAAAGTGGCACCGAGTCGGTGC |
| 3572 | sgRNA | TTTCCATTTACTCTTTTTGCGTTTTAGAGCTAGAAATAGCAAGTTAAAATAAGGCTA |
| | | GTCCGTTATCAACTTGAAAAAGTGGCACCGAGTCGGTGC |
| 3573 | sgRNA | CTGGTTTGCTTAAAAATGTGGTTTTAGAGCTAGAAATAGCAAGTTAAAATAAGGCTA |
| | | GTCCGTTATCAACTTGAAAAAGTGGCACCGAGTCGGTGC |
| 3574 | sgRNA | CACCTATAGATTACTATCCTGTTTTAGAGCTAGAAATAGCAAGTTAAAATAAGGCTA |
| | | GTCCGTTATCAACTTGAAAAAGTGGCACCGAGTCGGTGC |
| 3575 | sgRNA | TATAGATTACTATCCTGGTTGTTTTAGAGCTAGAAATAGCAAGTTAAAATAAGGCTA |
| | | GTCCGTTATCAACTTGAAAAAGTGGCACCGAGTCGGTGC |
| 3576 | sgRNA | TTTTTGCTGATGGCACATTCGTTTTAGAGCTAGAAATAGCAAGTTAAAATAAGGCTA |
| | | GTCCGTTATCAACTTGAAAAAGTGGCACCGAGTCGGTGC |
| 3577 | sgRNA | TCAGCATTAAACATTTCAAAGTTTTAGAGCTAGAAATAGCAAGTTAAAATAAGGCTA |
| | | GTCCGTTATCAACTTGAAAAAGTGGCACCGAGTCGGTGC |
| 3578 | sgRNA | GCACCTATAGATTACTATCCGTTTTAGAGCTAGAAATAGCAAGTTAAAATAAGGCTA |
| | | GTCCGTTATCAACTTGAAAAAGTGGCACCGAGTCGGTGCTTTT |
| 3579 | sgRNA | CTCTTTTCACTCAAGGTCTTGTTTTAGAGCTAGAAATAGCAAGTTAAAATAAGGCTA |
| | | GTCCGTTATCAACTTGAAAAAGTGGCACCGAGTCGGTGCTTTT |
| 3580 | sgRNA | GAAGTTGCTCTTTTCACTCAGTTTTAGAGCTAGAAATAGCAAGTTAAAATAAGGCTA |
| | | GTCCGTTATCAACTTGAAAAAGTGGCACCGAGTCGGTGCTTTT |
| 3581 | sgRNA | TCCTGGTTTGCTTAAAAATGGTTTTAGAGCTAGAAATAGCAAGTTAAAATAAGGCTA |
| | | GTCCGTTATCAACTTGAAAAAGTGGCACCGAGTCGGTGCTTTT |
| 3582 | sgRNA | GGTCTTGGGTATGCACCTATGTTTTAGAGCTAGAAATAGCAAGTTAAAATAAGGCTA |
| | | GTCCGTTATCAACTTGAAAAAGTGGCACCGAGTCGGTGCTTTT |
| 3583 | sgRNA | AGCATTAAACATTTCAAAAGGTTTTAGAGCTAGAAATAGCAAGTTAAAATAAGGCTA |
| | | GTCCGTTATCAACTTGAAAAAGTGGCACCGAGTCGGTGCTTTT |
| 3584 | sgRNA | TTTCCATTTACTCTTTTTGCGTTTTAGAGCTAGAAATAGCAAGTTAAAATAAGGCTA |
| | | GTCCGTTATCAACTTGAAAAAGTGGCACCGAGTCGGTGCTTTT |
| 3585 | sgRNA | CTGGTTTGCTTAAAAATGTGGTTTTAGAGCTAGAAATAGCAAGTTAAAATAAGGCTA |
| | | GTCCGTTATCAACTTGAAAAAGTGGCACCGAGTCGGTGCTTTT |
| 3586 | sgRNA | CACCTATAGATTACTATCCTGTTTTAGAGCTAGAAATAGCAAGTTAAAATAAGGCTA |
| | | GTCCGTTATCAACTTGAAAAAGTGGCACCGAGTCGGTGCTTTT |
| 3587 | sgRNA | TATAGATTACTATCCTGGTTGTTTTAGAGCTAGAAATAGCAAGTTAAAATAAGGCTA |
| | | GTCCGTTATCAACTTGAAAAAGTGGCACCGAGTCGGTGCTTTT |
| 3588 | sgRNA | TTTTTGCTGATGGCACATTCGTTTTAGAGCTAGAAATAGCAAGTTAAAATAAGGCTA |
| | | GTCCGTTATCAACTTGAAAAAGTGGCACCGAGTCGGTGCTTTT |
| 3589 | sgRNA | TCAGCATTAAACATTTCAAAGTTTTAGAGCTAGAAATAGCAAGTTAAAATAAGGCTA |
| | | GTCCGTTATCAACTTGAAAAAGTGGCACCGAGTCGGTGCTTTT |

TABLE 64
Spacer screen for spacers near the IVS4 + 4A -> T mutation in
human FANCC gene

| PAM | Spacer SEQ ID No. | sgRNA SEQ ID No. | %Indel frequency |
|---|---|---|---|
| NGG | 2619 | 3578 | 34.50% |
| NGG | 2616 | 3579 | 30.90% |
| NGG | 2618 | 3580 | 31.40% |
| NGA | 2628 | 3581 | 7.49% |
| NGA | 2622 | 3582 | 2.23% |
| NGA | 2573 | 3583 | 0.09% |
| NGA | 2895 | 3584 | 0.10% |
| NG | 2617 | 3585 | 0.49% |
| NG | 2613 | 3586 | 2.47% |
| NG | 2624 | 3587 | 10.64% |
| NG | 2896 | 3588 | 13.67% |
| NG | 2569 | 3589 | 0.22% |

### Example 3. Installing IVS4+4 A->T Mutation in Wildtype FANCC Gene

PEgRNAs were designed to have a spacer having the sequence of SEQ ID NO: 2619, PBS length of 8, 12, or 14 nt, and RTTs for incorporation of the IVS4+4 A->T mutation in wildtype FANCC gene. For each installation PEgRNA, a corresponding PEgRNA would have the same spacer and PBS sequences, and a RTT sequence of the same length but designed to correct the IVS4+4A->T mutation. The PEgRNAs were assembled as described in the third cloning method in Example 1.

For PE2 editing strategy, HEK293T cells were co-transfected with a plasmid expressing a prime editor fusion protein and a plasmid encoding a PEgRNA for installation of IVS4+4A->T mutation in the FANCC gene, and gDNA was harvested for sequencing with Miseq as described in Example 1. A non-transfection control was included, where the HEK293T cells were not transfected with any PEgRNA or prime editor-encoding polynucleotide.

Sequences of the PEgRNAs, corresponding components, and editing results are summarized in Table 65. Indels were not detected in any of the samples tested.

TABLE 65
Prime editing for installation of IVS4 + 4 A→T
mutation in wildtype FANCC (PE2 system)

| | PBS | | | |
|---|---|---|---|---|
| | SEQ ID | PBS | RTT | |
| PEgRNA sequence¹ | No. | length | length | %editing |
| Non-transfection control | NA | NA | NA | 0.05% |
| GCACCTATAGATTACTATCCGTTTTAGA | 3094 | 8 | 27 | 0.11% |
| GCTAGAAATAGCAAGTTAAAATAAGGCT | | | | |
| AGTCCGTTATCAACTTGAAAAAGTGGCA | | | | |
| CCGAGTCGGTGCGGCTATACACACATTT | | | | |
| TTAAGCAAACCAGGATAGTAATC (SEQ | | | | |
| ID NO: 3812) | | | | |
| GCACCTATAGATTACTATCCGTTTTAGA | 3094 | 8 | 29 | 0.11% |
| GCTAGAAATAGCAAGTTAAAATAAGGCT | | | | |
| AGTCCGTTATCAACTTGAAAAAGTGGCA | | | | |
| CCGAGTCGGTGCGGCTAAATACACACAT | | | | |
| TTTTAAGCAAACCAGGATAGTAATC | | | | |
| (SEQ ID NO: 3813) | | | | |
| GCACCTATAGATTACTATCCGTTTTAGA | 3094 | 8 | 31 | 0.11% |
| GCTAGAAATAGCAAGTTAAAATAAGGCT | | | | |
| AGTCCGTTATCAACTTGAAAAAGTGGCA | | | | |
| CCGAGTCGGTGCGGCTTTAAATACACAC | | | | |
| ATTTTTAAGCAAACCAGGATAGTAATC | | | | |
| (SEQ ID NO: 3814) | | | | |
| GCACCTATAGATTACTATCCGTTTTAGA | 3094 | 8 | 33 | 0.09% |
| GCTAGAAATAGCAAGTTAAAATAAGGCT | | | | |
| AGTCCGTTATCAACTTGAAAAAGTGGCA | | | | |
| CCGAGTCGGTGCGGCTTTTTAAATACAC | | | | |
| ACATTTTTAAGCAAACCAGGATAGTAAT | | | | |
| C (SEQ ID NO: 3815) | | | | |
| GCACCTATAGATTACTATCCGTTTTAGA | 3098 | 12 | 27 | 0.09% |
| GCTAGAAATAGCAAGTTAAAATAAGGCT | | | | |
| AGTCCGTTATCAACTTGAAAAAGTGGCA | | | | |
| CCGAGTCGGTGCGGCTATACACACATTT | | | | |
| TTAAGCAAACCAGGATAGTAATCTATA | | | | |
| (SEQ ID NO: 3816) | | | | |
| GCACCTATAGATTACTATCCGTTTTAGA | 3098 | 12 | 29 | 0.05% |
| GCTAGAAATAGCAAGTTAAAATAAGGCT | | | | |
| AGTCCGTTATCAACTTGAAAAAGTGGCA | | | | |
| CCGAGTCGGTGCGGCTAAATACACACAT | | | | |
| TTTTAAGCAAACCAGGATAGTAATCTAT | | | | |
| A (SEQ ID NO: 3817) | | | | |
| GCACCTATAGATTACTATCCGTTTTAGA | 3098 | 12 | 31 | 0.10% |
| GCTAGAAATAGCAAGTTAAAATAAGGCT | | | | |
| AGTCCGTTATCAACTTGAAAAAGTGGCA | | | | |
| CCGAGTCGGTGCGGCTTTAAATACACAC | | | | |
| ATTTTTAAGCAAACCAGGATAGTAATCT | | | | |
| ATA (SEQ ID NO: 3818) | | | | |
| GCACCTATAGATTACTATCCGTTTTAGA | 3098 | 12 | 33 | 0.07% |
| GCTAGAAATAGCAAGTTAAAATAAGGCT | | | | |
| AGTCCGTTATCAACTTGAAAAAGTGGCA | | | | |
| CCGAGTCGGTGCGGCTTTTTAAATACAC | | | | |
| ACATTTTTAAGCAAACCAGGATAGTAAT | | | | |
| CTATA (SEQ ID NO: 3819) | | | | |
| GCACCTATAGATTACTATCCGTTTTAGA | 3100 | 14 | 27 | 0.09% |
| GCTAGAAATAGCAAGTTAAAATAAGGCT | | | | |
| AGTCCGTTATCAACTTGAAAAAGTGGCA | | | | |
| CCGAGTCGGTGCGGCTATACACACATTT | | | | |
| TTAAGCAAACCAGGATAGTAATCTATAG | | | | |
| G (SEQ ID NO: 3820) | | | | |
| GCACCTATAGATTACTATCCGTTTTAGA | 3100 | 14 | 29 | 0.08% |
| GCTAGAAATAGCAAGTTAAAATAAGGCT | | | | |
| AGTCCGTTATCAACTTGAAAAAGTGGCA | | | | |
| CCGAGTCGGTGCGGCTAAATACACACAT | | | | |
| TTTTAAGCAAACCAGGATAGTAATCTAT | | | | |
| AGG (SEQ ID NO: 3821) | | | | |
| GCACCTATAGATTACTATCCGTTTTAGA | 3100 | 14 | 31 | 0.10% |
| GCTAGAAATAGCAAGTTAAAATAAGGCT | | | | |
| AGTCCGTTATCAACTTGAAAAAGTGGCA | | | | |
| CCGAGTCGGTGCGGCTTTAAATACACAC | | | | |
| ATTTTTAAGCAAACCAGGATAGTAATCT | | | | |
| ATAGG (SEQ ID NO: 3822) | | | | |

1. All PEgRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond.

Separately, PEgRNAs that have the sequences of SEQ ID NOs: 3816 and 3817 were also tested for PE3 editing strategy. Two ngRNAs having the sequence of SEQ ID NOs: 2899 and 2900 were assembled using the same method as the PEgRNAs. Sequences of the PEgRNAs and ngRNAs and editing results are summarized in Table 66. Indels were not detected in any of the samples tested.

TABLE 66
Prime editing for installation of IVS4 + 4 A -> T mutation in
wildtype FANCC (PE3 system)

| PEgRNA | ngRNA | ngRNA spacer | nick-to-nick | % editing |
|---|---|---|---|---|
| SEQ ID No.¹ | SEQ ID No.¹ | SEQ ID No. | distance | efficiency |
| 3816 | 2899 | 2886 | 108 | 0.19% |
| 3816 | 2900 | 2892 | 111 | 0.13% |
| 3817 | 2899 | 2886 | 108 | 0.22% |
| 3817 | 2900 | 2892 | 111 | 0.26% |

2. All PEgRNAs and ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond.

### Example 4: Editing of the IVS4+4A->T Mutation in Human FANCC Gene Using Prime Editing

A clonal cell line homozygous for the FANCC IVS4+4 A->T mutation as described in Examples 1 and 3 is used for examination of correction of IVS4+4 A->T mutation. Alternatively, FANCC IVS4+4 A>T cell line generated by lentiviral transfection as described above can be used for examination of correction of IVS4+4 A>T mutation.

Exemplary sequences of PEgRNAs and ngRNAs are provided in Tables 35-52 for PE2 and PE3 editing strategies. The clonal HEK293T cell line carrying the IVS4+4 A->T mutation is transfected with polynucleotides encoding a prime editor fusion protein, a pegRNA (PE2 editing strategy) and optionally a ngRNA (PE3 editing strategy) as described in Example 1. Seventy-two hours after transfection, gDNA is extracted and editing efficiency is determined by Miseq as described in Example 1.

### Example 5: Editing of the 322delG Mutation at the Endogenous FANCC Genomic Site in HEK293T Cells Using Prime Editing

A homozygous HEK293T cell line that carries the 322delG mutation in FANCC was generated in accordance with methods in Example 1 using PEgRNAs that encode the 322delG mutation in the RTT. PEgRNAs designed for correction of the 322delG mutation were chemically synthesized by Integrated DNA Technologies (IDT). The synthesized PEgRNAs were chemically modified at the 5′ end and the 3′ end: the three 5′ most nucleotides were modified to phosphorothioated 2′-O-methyl nucleotides. The three consecutive nucleotides that precede the 3′ most nucleotide (i.e. three consecutive nucleotides immediately 5′ of the last nucleotide at the 3′ end) were also modified to phosphorothioated 2′-O-methyl nucleotides. For PE2 editing strategy, HEK293T cells were co-transfected with mRNA encoding a prime editor fusion protein and a PEgRNA, and gDNA was harvested for sequencing with Miseq as described in Example 1. A “mock” non-transfection control was included, where the HEK293T cells were not transfected with any PEgRNA or prime editor-encoding polynucleotide.

A total of 95 PEgRNAs were tested, each containing a spacer having the sequence of SEQ ID NO: 35,139,212, or 213 (nick-to-edit distance 7, 1, 16, and 28nt, respectively), and a PBS of 8 to 14 nt in length. Each of the PEgRNA RTTs tested encoded for an insertion of G or C at the position corresponding to the c.67 (322delG) deletion to correct the deletion. 23 of the tested PEgRNAs also encoded for a synonymous AGG to AAG PAM silencing edit, as indicated in Table 67.

The PEgRNA components and editing results are summarized in Table 67. Editing efficiency above the non-transfection control value was observed in cells edited with PEgRNAs having different spacers. For PEgRNAs having the spacer sequence of SEQ ID NO: 35 or SEQ ID NO: 139, editing levels significantly higher than that observed in the non-transfection control was observed, with higher editing efficiency observed in cells edited with PEgRNAs having the spacer of SEQ ID NO: 139 (nick-to-edit distance=1). Successful prime editing was observed for PEgRNAs both with and without the AGG to AAG PAM silencing edit. Compared to corresponding PEgRNAs without the PAM silencing edit, increased editing efficiency was observed in treatment with 21 of 23 PEgRNAs that contained the PAM silencing edit. Significantly increased editing efficiency was observed with PEgRNAs that contained a 12, 14, or 16nt long RTT and the PAM silencing edit.

TABLE 67
Prime Editing at c.67del (322delG) mutation
site in human FANCC gene (PE2 system)

| PEgRNA | spacer | PBS | | RTT | | | |
|---|---|---|---|---|---|---|---|
| SEQ ID | SEQ ID | SEQ ID | PBS | SEQ ID | RTT | | |
| No.¹ | No. | No. | length | No.² | length | % Editing | % Indels |
| Mock | NA | NA | NA | NA | NA | 0.05% | 0% |
| 965 | 35 | 923 | 8 | 936 | 11 | 4.83% | 0.03% |
| 967 | 35 | 923 | 8 | 939 | 14 | 5.56% | 0% |
| 971 | 35 | 923 | 8 | 941 | 16 | 3.18% | 0% |
| 976 | 35 | 923 | 8 | 943 | 18 | 2.80% | 0.03% |
| 982 | 35 | 923 | 8 | 384 | 20 | 2.34% | 0.02% |
| 966 | 35 | 925 | 10 | 936 | 11 | 5.28% | 0.04% |
| 970 | 35 | 925 | 10 | 939 | 14 | 3.89% | 0.03% |
| 975 | 35 | 925 | 10 | 941 | 16 | 3.63% | 0% |
| 981 | 35 | 925 | 10 | 943 | 18 | 2.97% | 0.01% |
| 988 | 35 | 925 | 10 | 384 | 20 | 1.85% | 0.02% |
| 968 | 35 | 927 | 12 | 936 | 11 | 5.55% | 0.03% |
| 974 | 35 | 927 | 12 | 939 | 14 | 4.88% | 0.04% |
| 980 | 35 | 927 | 12 | 941 | 16 | 4.87% | 0.06% |
| 987 | 35 | 927 | 12 | 943 | 18 | 2.93% | 0.01% |
| 993 | 35 | 927 | 12 | 384 | 20 | 2.72% | 0% |
| 972 | 35 | 929 | 14 | 936 | 11 | 5.30% | 0.04% |
| 979 | 35 | 929 | 14 | 939 | 14 | 7.54% | 0.05% |
| 986 | 35 | 929 | 14 | 941 | 16 | 6.97% | 0.07% |
| 992 | 35 | 929 | 14 | 943 | 18 | 7.41% | 0.10% |
| 998 | 35 | 929 | 14 | 384 | 20 | 7.08% | 0.06% |
| 219 | 139 | 145 | 8 | 157 | 10 | 8% | 0.11% |
| 222 | 139 | 145 | 8 | 160 | 12 | 11.58% | 0.04% |
| 224 | 139 | 145 | 8 | 161* | 12 | 12.41% | 0.06% |
| 227 | 139 | 145 | 8 | 163 | 14 | 5.64% | 0.11% |
| 232 | 139 | 145 | 8 | 164* | 14 | 16.91% | 0.01% |
| 237 | 139 | 145 | 8 | 166 | 16 | 4.86% | 0.02% |
| 245 | 139 | 145 | 8 | 167* | 16 | 14.20% | 0% |
| 299# | 139 | 145 | 8 | 182* | 30 | 5.10% | 0.01% |
| 301# | 139 | 145 | 8 | 185* | 32 | 8.20% | 0.01% |
| 221 | 139 | 147 | 10 | 157 | 10 | 10.74% | 0.07% |
| 223 | 139 | 147 | 10 | 158* | 10 | 6.54% | 0% |
| 226 | 139 | 147 | 10 | 160 | 12 | 6.99% | 0% |
| 231 | 139 | 147 | 10 | 161* | 12 | 13.11% | 0.05% |
| 236 | 139 | 147 | 10 | 163 | 14 | 8.44% | 0% |
| 244 | 139 | 147 | 10 | 164* | 14 | 20.09% | 0% |
| 250 | 139 | 147 | 10 | 166 | 16 | 7.06% | 0.06% |
| 259 | 139 | 147 | 10 | 167* | 16 | 17.08% | 0% |
| 300# | 139 | 147 | 10 | 182* | 30 | 9.20% | 0% |
| 303# | 139 | 147 | 10 | 185* | 32 | 6.78% | 0% |
| 225 | 139 | 149 | 12 | 157 | 10 | 10.35% | 0.02% |
| 230 | 139 | 149 | 12 | 158* | 10 | 6.93% | 0.03% |
| 235 | 139 | 149 | 12 | 160 | 12 | 7.21% | 0.03% |
| 243 | 139 | 149 | 12 | 161* | 12 | 12.04% | 0.01% |
| 249 | 139 | 149 | 12 | 163 | 14 | 4.51% | 0.02% |
| 258 | 139 | 149 | 12 | 164* | 14 | 16.23% | 0.02% |
| 264 | 139 | 149 | 12 | 166 | 16 | 9.07% | 0.02% |
| 274 | 139 | 149 | 12 | 167* | 16 | 10.86% | 0.02% |
| 302# | 139 | 149 | 12 | 182* | 30 | 7.46% | 0% |
| 306# | 139 | 149 | 12 | 185* | 32 | 4.35% | 0.01% |
| 234 | 139 | 151 | 14 | 157 | 10 | 6.54% | 0.01% |
| 242 | 139 | 151 | 14 | 158* | 10 | 6.60% | 0.01% |
| 248 | 139 | 151 | 14 | 160 | 12 | 7.84% | 0% |
| 257* | 139 | 151 | 14 | 161* | 12 | 14.23% | 0.01% |
| 263 | 139 | 151 | 14 | 163 | 14 | 5.57% | 0.01% |
| 273 | 139 | 151 | 14 | 164* | 14 | 14.67% | 0% |
| 279 | 139 | 151 | 14 | 166 | 16 | 3.76% | 0% |
| 286 | 139 | 151 | 14 | 167* | 16 | 12.36% | 0% |
| 305# | 139 | 151 | 14 | 182* | 30 | 7.05% | 0.01% |
| 308# | 139 | 151 | 14 | 185* | 32 | 2.06% | 0.01% |
| 1403 | 212 | 1238 | 8 | 28 | 20 | 0.16% | 0.02% |
| 1405 | 212 | 1238 | 8 | 1288 | 22 | 0.30% | 0.07% |
| 1409 | 212 | 1238 | 8 | 1306 | 25 | 0.47% | 0.02% |
| 1415 | 212 | 1238 | 8 | 1318 | 27 | 0.16% | 0.02% |
| 1421 | 212 | 1238 | 8 | 1330 | 29 | 0.13% | 0% |
| 1404 | 212 | 1240 | 10 | 28 | 20 | 0.17% | 0.07% |
| 1407 | 212 | 1240 | 10 | 1288 | 22 | 0.50% | 0.15% |
| 1414 | 212 | 1240 | 10 | 1306 | 25 | 1.10% | 0% |
| 1420 | 212 | 1240 | 10 | 1318 | 27 | 0.30% | 0.04% |
| 1427 | 212 | 1240 | 10 | 1330 | 29 | 0.17% | 0.34% |
| 1406 | 212 | 1242 | 12 | 28 | 20 | 0.72% | 0.01% |
| 1411 | 212 | 1242 | 12 | 1288 | 22 | 0.64% | 0.03% |
| 1419 | 212 | 1242 | 12 | 1306 | 25 | 0.95% | 0.01% |
| 1426 | 212 | 1242 | 12 | 1318 | 27 | 0.21% | 0% |
| 1432 | 212 | 1242 | 12 | 1330 | 29 | 0.31% | 0.01% |
| 1410 | 212 | 1244 | 14 | 28 | 20 | 0.13% | 0.01% |
| 1416 | 212 | 1244 | 14 | 1288 | 22 | 0.78% | 0.04% |
| 1425 | 212 | 1244 | 14 | 1306 | 25 | 0.55% | 0.07% |
| 1431 | 212 | 1244 | 14 | 1318 | 27 | 0.18% | 0.04% |
| 1436 | 212 | 1244 | 14 | 1330 | 29 | 0.17% | 0% |
| 1541 | 213 | 1471 | 8 | 1500 | 32 | 0.10% | 0% |
| 1543 | 213 | 1471 | 8 | 1508 | 34 | 0.25% | 0.02% |
| 1547 | 213 | 1471 | 8 | 1520 | 37 | 0.50% | 0.04% |
| 1554 | 213 | 1471 | 8 | 1528 | 39 | 0.48% | 0% |
| 1542 | 213 | 1473 | 10 | 1500 | 32 | 0.08% | 0.06% |
| 1545 | 213 | 1473 | 10 | 1508 | 34 | 0.71% | 0% |
| 1553 | 213 | 1473 | 10 | 1520 | 37 | 0.45% | 0.01% |
| 1558 | 213 | 1473 | 10 | 1528 | 39 | 0.37% | 0% |
| 1544 | 213 | 1475 | 12 | 1500 | 32 | 0.09% | 0% |
| 1549 | 213 | 1475 | 12 | 1508 | 34 | 0.30% | 0.03% |
| 1557 | 213 | 1475 | 12 | 1520 | 37 | 1.20% | 0.02% |
| 1563 | 213 | 1475 | 12 | 1528 | 39 | 0.46% | 0% |
| 1552 | 213 | 1476 | 13 | 1508 | 34 | 0.52% | 0.01% |
| 1548 | 213 | 1477 | 14 | 1500 | 32 | 0.07% | 0% |
| 1562 | 213 | 1477 | 14 | 1520 | 37 | 0.53% | 0.03% |
| 1567 | 213 | 1477 | 14 | 1528 | 39 | 0.53% | 0.01% |

1. All PEgRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The PEgRNA used experimentally for PEgRNA sequences that are annotated with “#” further contained an additional 5′ G, a linker sequence (AACATTGA), and a 3′ hairpin having the sequence of SEQ ID NO: 3591 (i.e. from 5′ to 3′: additional nucleotide G, indicated Spacer sequence, a gRNA core having the sequence of SEQ ID NO: 3666, the indicated RTT sequence, the indicated PBS sequence, the linker sequence, and the 3′ hairpin according to SEQ ID NO: 3591).
2. *: RTT encodes a synonymous AGG to AAG PAMsilencing edit.

9 PEgRNAs from the PE2 editing experiment above were also tested with a PE3 editing strategy. HEK293T cells were co-transected with mRNA encoding a prime editor fusion protein, synthesized PEgRNA and ngRNA, and gDNA was harvested for sequencing with Miseq as described in Example 1. The PEgRNA and ngRNA components and editing results are summarized in Tables 68-73. A non-transfection control indicates that the cells were not transfected with any PEgRNA or prime editor-encoding polynucleotide. Efficient editing was observed in cells edited with PEgRNAs having the spacer sequence of SEQ ID NO: 139 or SEQ ID NO: 35 paired with all ngRNAs tested, for both PEgRNAs with and without a PAM silencing mutation. Including an additional PAM silencing mutation in sequence encoded by the RTT appears to improve editing efficiency.

TABLE 68
Prime Editing at c.67del (322delG) mutation site in human
FANCC gene (PE3 system) with a PEgRNA having the spacer
as set forth in SEQ ID NO: 212 and ngRNAs

| PEgRNA | ngRNA | ngRNA | nick-to- | | |
|---|---|---|---|---|---|
| SEQ ID | spacer SEQ | SEQ ID | nick | % | % |
| No.¹ | ID No. | No.1 | distance | Editing | Indels |
| 1414 | 47 | 49# | 58 | 3.03% | 0.45% |
| | 31 | 51 | 31 | 4.43% | 0.30% |
| | 35 | 50 | 37 | 3.82% | 1% |
| | 28 | 52# | 19 | 1.37% | 0.32% |

1. PEgRNA and all ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The ngRNAs used experimentally for ngRNA sequences that are annotated with “#” further contained an additional 5′ G, a linker sequence (AACATTGA), and a 3′ hairpin having the sequence of SEQ ID NO: 3591 (i.e. from 5′ to 3′: additional nucleotide G, indicated Spacer sequence, a gRNA core having the sequence of SEQ ID NO: 3666,the linker sequence, and the 3′ hairpin according to SEQ ID NO: 3591).

TABLE 69
Prime Editing at c.64del (322delG) mutation site in human
FANCC gene (PE3 system) with a PEgRNA having the spacer
as set forth in SEQ ID NO: 139 and ngRNAs

| PEgRNA | ngRNA | ngRNA | nick-to- | | |
|---|---|---|---|---|---|
| SEQ ID | spacer SEQ | SEQ ID | nick | % | % |
| No.^{1,2} | ID No. | No.¹ | distance | Editing | Indels |
| 236 | 212 | 323 | 22 | 15.91% | 0.87% |
| | 213 | 324 | 34 | 18.35% | 1.12% |
| | 203 | 325# | 35 | 16% | 0.83% |
| | 209 | 326 | 46 | 22.79% | 1.31% |
| | 195 | 327# | 68 | 22.23% | 1.23% |
| | 199 | 328# | 77 | 30.14% | 1.63% |
| | 196 | 329# | 97 | 29.14% | 1.35% |
| 244* | 212 | 323 | 22 | 10.58% | 0.37% |
| | 213 | 324 | 34 | 12.56% | 0.56% |
| | 203 | 325# | 35 | 9.45% | 0.40% |
| | 209 | 326 | 46 | 18.16% | 0.66% |
| | 195 | 327# | 68 | 20.18% | 0.67% |
| | 199 | 328# | 77 | 26.59% | 0.73% |
| | 196 | 329# | 97 | 50.48% | 0.97% |

1. All PEgRNAs and ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The ngRNAs used experimentally for ngRNA sequences that are annotated with “#” further contained an additional 5′G, a linker sequence (AACATGA), and a 3′ hairpin having the sequence of SEQ ID NO: 359] (i. e. from 5′ to 3′: additional nucleotide G, indicated Spacer sequence, a gRNA core having the sequence of SEQ ID NO: 3666,the linker sequence, and the 3′ hairpin according to SEQ ID NO: 359]).
2. *:PEgRNA contains an RTT that encodes a synonymous AGG to AAG PAM silencing edit.

TABLE 70
Prime Editing at c.64del (322delG) mutation site in human
FANCC gene (PE3 system) with a PEgRNA having the spacer
as set forth in SEQ ID NO: 139 and ngRNAs¹

| PEgRNA | ngRNA | ngRNA | nick-to- | | |
|---|---|---|---|---|---|
| SEQ ID | spacer | SEQ ID | nick | % | % |
| No.^{2,3} | SEQ ID | No.² | distance | Editing | Indels |
| Non- | NA | NA | N/A | 0.0999 | 3.0969 |
| transfection | | | | | |
| control | | | | | |
| 236 | 209 | 326 | 46 | 27.0167 | 15.3898 |
| 250 | 209 | 326 | 46 | 23.9985 | 13.9927 |
| 244* | 209 | 326 | 46 | 34.4241 | 23.0356 |
| 259* | 209 | 326 | 46 | 34.6201 | 22.1377 |

1. Experiment was performed separately from the experiment in Table 69 (PEgRNAs and ngRNAs synthesized by different vendors).
2. All PEgRNAs and ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The ngRNAs used experimentally for PEgRNA sequences annotated with “#” further contained an additional 5′ G, a linker sequence (AACATTGA), and a 3′ hairpin having the sequence of SEQ ID NO: 359] (i.e. from 5′ to 3′: additional nucleotide G, indicated Spacer sequence, a gRNA core having the sequence of SEQ ID NO: 3666, the linker sequence, and the 3′ hairpin according to SEQ ID NO: 359]).
3. *: PEgRNA contains an RTT that encodes a synonymous AGG to AAG PAM silencing edit.

TABLE 71
Prime Editing at c.67del (322delG) mutation site in human
FANCC gene (PE3 system) with a PEgRNA having the spacer
as set forth in SEQ ID NO: 213 and ngRNAs

| PEgRNA | ngRNA | ngRNA | nick-to- | | |
|---|---|---|---|---|---|
| SEQ ID | spacer SEQ | SEQ ID | nick | % | % |
| No.¹ | ID No. | No.¹ | distance | Editing | Indels |
| 1558 | 47 | 49# | 70 | 1.72% | 0.39% |
| | 31 | 51 | 43 | 2.46% | 0.25% |
| | 35 | 50 | 49 | 3.33% | 0.81% |
| | 28 | 52# | 31 | 0.94% | 0.24% |

1. PEgRNA and all ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The ngRNAs used experimentally for PEgRNA sequences annotated with “#” further contained an additional 5′ G, a linker sequence (AACATTGA), and a 3′ hairpin having the sequence of SEQ ID NO: 359] (i. e. from 5′ to 3′: additional nucleotide G, indicated Spacer sequence, a gRNA core having the sequence of SEQ ID NO: 3666, the linker sequence, and the 3′ hairpin according to SEQ ID NO: 359]).

TABLE 72
Prime Editing at c.67del (322delG) mutation site in human
FANCC gene (PE3 system) with a PERNA having the spacer
as set forth in SEQ ID NO: 35 and ngRNAs

| PEgRNA | ngRNA | ngRNA | nick-to- | | |
|---|---|---|---|---|---|
| SEQ ID | spacer SEQ | SEQ ID | nick | % | % |
| No.¹ | ID No. | No.¹ | distance | Editing | Indels |
| 968 | 212 | 323 | 28 | 18.37% | 4.08% |
| | 213 | 324 | 40 | 20.96% | 5.17% |
| | 203 | 325# | 41 | 18.81% | 1.99% |
| | 209 | 326 | 52 | 24.22% | 3.06% |
| | 195 | 327# | 74 | 25.93% | 2.61% |
| | 199 | 328# | 83 | 27.62% | 2.56% |
| | 196 | 329# | 103 | 15.43% | 1.02% |
| 986 | 212 | 323 | 28 | 22.11% | 2.38% |
| | 213 | 324 | 40 | 26.69% | 2.58% |
| | 203 | 325# | 41 | 24.80% | 1.56% |
| | 209 | 326 | 52 | 29.93% | 2.48% |
| | 195 | 327# | 74 | 30.42% | 1.72% |
| | 199 | 328# | 83 | 35.32% | 2.18% |
| | 196 | 329# | 103 | 22.23% | 1.12% |

1. All PEgRNAs and ngRNAs used experimentally contained a 3′ IT T end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The ngRNAs used experimentally for PEgRNA sequences annotated with “#” further contained an additional 5′ G, a linker sequence (AACATTGA), and a 3′ hairpin having the sequence of SEQ ID NO: 3591 (i.e. from 5′ to 3′: additional nucleotide G, indicated Spacer sequence, a gRNA core having the sequence of SEQ ID NO: 3666,the linker sequence, and the 3′ hairpin according to SEQ ID NO: 3591).

TABLE 73
Prime Editing at c.67del (322delG) mutation site in human
FANCC gene (PE3 system) with a PEgRNA having the spacer
as set forth in SEQ ID NO: 35 and ngRNAs¹

| PEgRNA | ngRNA | ngRNA | nick-to- | | |
|---|---|---|---|---|---|
| SEQ ID | spacer SEQ | SEQ ID | nick | editing | indel |
| No.² | ID No. | No.² | distance | efficiency | frequency |
| Non- | NA | NA | N/A | 0.0999 | 3.0969 |
| transfection | | | | | |
| control | | | | | |
| 970 | 209 | 326 | 52 | 20.8879 | 13.3835 |

1. Experiment was performed separately from the experiment in Table 73 (PEgRNAs and ngRNAs synthesized by different vendors).
2. All PEgRNAs and ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond.

### Example 6: Prime Editing of the 322delG Mutation at Endogenous FANCC Site in Lymphoblastoid Cell Line (LCL) from Individual Carrying the Mutation

PEgRNAs having a spacer according to SEQ ID NO: 139 or SEQ ID NO: 35 and ngRNAs were chemically synthesized and purified as described in Example 1. Lymphoblastoid cells (LCLs) from a human donor that carries the 322delG mutation were used for editing. The patient's FANCC gene is heterozygotic at the 322delG mutation site; the genetic makeup was unclear for other mutations in the FANCC gene that may impact the DNA repair function. For each Prime Editing composition treatment, the LCLs were mixed with mRNA encoding a Prime Editor fusion protein and a PEgRNA (along with a ngRNA for PE3 editing strategy) in a Maxcyte cartridge, and electroporated using the MaxCyte electroporation system following the Manufacturer's protocol. On day 4 and day 11 after electroporation, genomic DNA was extracted and analyzed with Miseq sequencing. Because the donor only carries one copy of FANCC that harbors the 322delG mutation, editing efficiency was estimated as (Miseq editing %)-50%.

The PEgRNA and ngRNA components and editing results are summarized in Table 74. A non-transfection control indicates that the cells were not transfected with any PEgRNA or prime editor-encoding polynucleotide. The PEgRNA sequences used are summarized in Table 75.

TABLE 74
Prime Editing at c.67del (322delG) mutation site in
FANCC gene in patient derived LCLs (PE3 system)

| | | ngRNA | | | | | |
|---|---|---|---|---|---|---|---|
| PEgRNA | ngRNA | spacer | nick-to- | | | | |
| SEQ ID | SEQ ID | SEQ ID | nick | % Editing | % Indels | % Editing | % Indels |
| No.¹ | No.² | No. | distance | day 4 | day 4 | day 11 | day 11 |
| non- | NA | NA | NA | −0.77% | 0.12% | 0.12% | 0.12% |
| transfection | | | | | | | |
| control | | | | | | | |
| 236 | PE2 | NA | NA | 0.02% | 0.18% | 1.22% | 0.24% |
| 236 | 326 | 209 | 46 | 10.84% | 2.72% | 9.30% | 3.01% |
| 236 | 327# | 195 | 68 | 2.66% | 0.65% | 5.58% | 0.69% |
| 236 | 328# | 199 | 77 | 6.33% | 1.23% | 5.16% | 1.06% |
| 236 | 329# | 196 | 97 | 3.01% | 0.80% | 8.99% | 0.15% |
| 250 | PE2 | N/A | N/A | 0.10% | 0.20% | 0.82% | 0.21% |
| 250 | 326 | 209 | 46 | 8.03% | 3.07% | 4.43% | 3.17% |
| 250 | 327# | 195 | 68 | 3.94% | 1.21% | 4.36% | 1.51% |
| 250 | 328# | 199 | 77 | 2.73% | 0.69% | 1.37% | 0.95% |
| 250 | 329# | 196 | 97 | 2.96% | 0.95% | 1.74% | 0.73% |
| 244* | PE2 | N/A | N/A | 1.28% | 0.12% | 0.02% | 0.10% |
| 244* | 326 | 209 | 46 | 5.33% | 2.17% | 4.88% | 1.94% |
| 244* | 327# | 195 | 68 | 1.77% | 0.51% | 0.84% | 0.69% |
| 244* | 328# | 199 | 77 | 3.05% | 0.82% | 2.11% | 0.80% |
| 244* | 329# | 196 | 97 | 1.47% | 0.57% | −0.08% | 0.45% |
| 259* | PE2 | N/A | N/A | −0.11% | 0.18% | −1.61% | 0.19% |
| 259* | 326 | 209 | 46 | 5.71% | 1.66% | 3.66% | 2.10% |
| 259* | 327# | 195 | 68 | 1.64% | 0.45% | 1.27% | 0.55% |
| 259* | 328# | 199 | 77 | 2.07% | 0.66% | 2.19% | 0.87% |
| 259* | 329# | 196 | 97 | 0.74% | 0.56% | 2.17% | 0.49% |
| 966 | PE2 | N/A | NA | −0.91% | 0.21% | 1.35% | 0.11% |
| 966 | 326 | 209 | 52 | 2.02% | 1.32% | 0.40% | 2.09% |
| 966 | 327# | 195 | 74 | 0.17% | 0.44% | 0.17% | 0.55% |
| 966 | 328# | 199 | 83 | 0.76% | 0.46% | 1.58% | 0.65% |
| 966 | 329# | 196 | 103 | 0.49% | 0.51% | 0.98% | 0.29% |
| 970 | PE2 | N/A | N/A | −0.11% | 0.16% | −1.45% | 0.13% |
| 970 | 326 | 209 | 52 | 3.42% | 1.47% | 4.03% | 1.97% |
| 970 | 327# | 195 | 74 | 1.93% | 0.65% | −0.75% | 0.89% |
| 970 | 328# | 199 | 83 | 2.25% | 0.35% | 0.41% | 0.51% |
| 970 | 329# | 196 | 103 | 1.90% | 0.48% | 0.72% | 0.36% |

1. All PEgRNAs used experimentally contained a 3′ TT IT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. “#” following the PEgRNA SEQ ID NO. indicates that the RTT of the PEgRNA encodes a synonymous AGG to AAG PAM silencing edit.
2. All ngRNAs used experimentally contained a 3′ TTTT end adaptation, and further contained chemical modifications 3′ mN*mN*mN*N and 5′mN*mN*mN*N modifications, where m indicates that the nucleotide contains a 2′-O-Me modification and a * indicates a phosphorothioate bond. The ngRNA used experimentally for PEgRNA sequences in Table 74 indicated with “#” further contained an additional 5′ G, a linker sequence (AACATTGA), and a 3′ hairpin having the sequence of SEQ ID NO: 3591 (i.e. from 5′ to 3′: additional nucleotide G, indicated Spacer sequence, a gRNA core having the sequence of SEQ ID NO: 3666, the indicated RTT sequence, the indicated PBS sequence, the linker sequence, and the 3′ hairpin according to SEQ ID NO: 3591). “PE2” indicates that a PE2 editing strategy was used (i.e. no ngRNA was used).

TABLE 75
Summary of PEgRNAs used in Table 74

| PERNA | PEgRNA | PBS | | RTT | |
|---|---|---|---|---|---|
| SEQ ID | spacer SEQ | SEQ | PBS | SEQ | RTT |
| No. | ID No. | ID No. | length | ID No. | length |
| 236 | 139 | 147 | 10 | 163 | 14 |
| 250 | 139 | 147 | 10 | 166 | 16 |
| 244 | 139 | 147 | 10 | 164 | 14 |
| 259 | 139 | 147 | 10 | 167 | 16 |
| 966 | 35 | 925 | 10 | 936 | 11 |
| 970 | 35 | 925 | 10 | 939 | 14 |

1. A prime editing guide RNA (PEgRNA) or a nucleic acid encoding the PEgRNA, wherein the PEgRNA comprises:
a. a spacer that is complementary to a search target sequence on a first strand of a FANCC gene wherein the spacer comprises at its 3′ end SEQ ID NO: 136;
b. a gRNA core capable of binding to a Cas9 protein; and
c. an extension arm comprising:
i. an editing template that comprises a region of complementarity to an editing target sequence on a second strand of the FANCC gene, and
ii. a primer binding site (PBS) that comprises at its 5′ end a sequence that is a reverse complement of nucleotides 10-14 of SEQ ID NO: 136,
wherein the first strand and second strand are complementary to each other,
wherein the editing target sequence on the second strand comprises or is complementary to a portion of the FANCC gene comprising a c.67del mutation, and
wherein the editing template encodes an insertion of a guanine nucleotide at a site corresponding to the c.67del mutation compared to the editing target sequence.

2. A prime editing guide RNA (PEgRNA), or a nucleic acid encoding the PEgRNA, wherein the PEgRNA comprises:
a. a spacer comprising at its 3′ end SEQ ID NO: 136;
b. a gRNA core capable of binding to a Cas9 protein; and
c. an extension arm comprising:
i. an editing template comprising at its 3′ end SEQ ID NO: 157 or 158, and
ii. a primer binding site (PBS) comprising at its 5′ end a sequence that is a reverse complement of nucleotides 10-14 of SEQ ID NO: 136.

3. The PEgRNA of claim 1, wherein the spacer comprises at its 3′ end any one of SEQ ID NOs: 137-141.

4. The PEgRNA of claim 3, wherein the spacer comprises at its 3′ end SEQ ID NO: 139.

5. The PEgRNA of claim 1, wherein the editing template comprises SEQ ID NO: 157 at its 3′ end.

6. The PEgRNA of claim 5, wherein the editing template comprises at its 3′ end SEQ ID NO: 159, 160, 162, 163, 165, 166, 168-181, 183, 184, 186, 187, or 189-194.

7. The PEgRNA of claim 1, wherein the editing template comprises SEQ ID NO: 158 at its 3′ end and encodes an AGG-to-AAG PAM silencing edit.

8. The PEgRNA of claim 7, wherein the editing template comprises at its 3′ end SEQ ID NO: 161, 164, 167, 182, 185, or 188.

9. The PEgRNA of claim 1, wherein the editing template is 10 to 40 nucleotides in length.

10. The PEgRNA of claim 9, wherein the editing template is 10 to 32 nucleotides in length.

11. The PEgRNA of claim 10, wherein the editing template is 12 to 16 nucleotides in length.

12. The PEgRNA of claim 1, wherein the PBS comprises at its 5′ end a sequence corresponding to sequence number 142.

13. The PEgRNA of claim 1, wherein the PBS comprises any one of sequence numbers 142-146, or any one of SEQ ID NOs: 147-156.

14. The PEgRNA of claim 1, wherein the PBS is 8 to 14 nucleotides in length.

15. The PEgRNA of claim 14, wherein the PBS is 14 nucleotides in length.

16. The PEgRNA of claim 1, wherein the gRNA core comprises SEQ ID NO: 3666.

17. The PEgRNA of claim 1, comprising a PEgRNA sequence selected from any one of SEQ ID NOs: 219-322 or 3592-3603.

18. A prime editing system comprising: (a) the PEgRNA or the nucleic acid of claim 1, and (b) a ngRNA, or a nucleic acid encoding the ngRNA, wherein the ngRNA comprises:
(i) a spacer comprising at its 3′ end a sequence corresponding to nucleotides 4-20 of any one of SEQ ID NOs: 4 or 195-218; and
(ii) an ngRNA core capable of binding a Cas9 protein.

19. The prime editing system of claim 18, wherein the spacer of the ngRNA comprises at its 3′ end any one of SEQ ID NOs: 4 or 195-218.

20. The prime editing system of claim 18, wherein the ngRNA core comprises the same sequence as the gRNA core.

21.-119. (canceled)
